(19) United States
(12) Patent Application Publication
(10) Pub. No.: US 2023/0158150 A1
(43) Pub. Date: May 25, 2023

(54) COMPOUND COMPRISING A NUCLEIC ACID AND A HALF-LIFE EXTENSION MOTIF

(71) Applicant: DTX PHARMA, INC., San Diego (US)

(72) Inventors: Arthur T. Suckow, San Diego, CA (US); Charles Allerson, San Diego, CA (US); Fabio C. Tucci, San Diego, CA (US)

(21) Appl. No.: 17/778,372

(22) PCT Filed: Nov. 25, 2020
(86) PCT No.: PCT/US2020/062358

§ 371 Date: May 19, 2022

Related U.S. Application Data
(60) Provisional application No. 62/940,835, filed on Nov. 26, 2019.

(51) Int. Cl. A61K 47/54; C12N 15/113

(52) U.S. Cl. CPC A61K 47/543; C12N 15/1137; A61K 47/542; C12N 2310/14; C12N 2310/3515; C12N 2320/51; C12N 2310/346; C12N 2320/30

(57) ABSTRACT

Disclosed herein are compounds including a nucleic acid (A), their preparation, and their use.

### CROSS-REFERENCES TO RELATED APPLICATIONS

This application claims the benefit of U.S. Provisional Patent Application No. 62/940,835, filed Nov. 26, 2019, which is incorporated herein in its entirety and for all purposes.

### REFERENCE TO A “SEQUENCE LISTING,” A TABLE, OR A COMPUTER PROGRAM LISTING APPENDIX SUBMITTED AS AN ASCII FILE

The Sequence Listing written in file DTX-003-01WO_ST25.TXT, created on Nov. 23, 2020, 1,174 bytes in size, machine format IBM-PC, MS Windows operating system, is hereby incorporated by reference.

### BACKGROUND

### Field

The present disclosure relates to the field of biologically active compounds including a nucleic acid. More specifically, the present disclosure relates to compounds including nucleic acids, their preparation, and their use.

### Background

Delivering therapeutic nucleic acids into cells remains a challenging area of research. Thus, there is a need for improved nucleic acid compounds and strategies of introducing such compounds into cells.

### BRIEF SUMMARY

Provided herein are, inter alia, compounds, or compounds including a nucleic acid (A) covalently bonded to a half-life extension motif (HLEM).

In an aspect, provided is a compound having a formula (I)

(HLEM)_{z}-A (I).

z is an integer from 1 to 5.

In embodiments, the half-life extension motif has the structure:

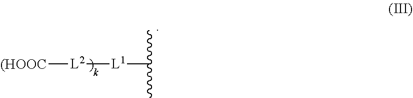

k is an integer from 1 to 5.

L¹ is independently a covalent linker. L² is independently an unsubstituted alkylene.

In embodiments, the nucleic acid is covalently bonded to an uptake motif (UM).

In embodiments, the compound has a formula (II):

(HLEM)_{z}-A-(UM)ₜ (II).

t is an integer from 1 to 5.

In embodiments, the uptake motif independently has the structure:

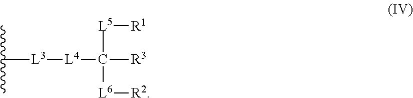

L³ and L⁴ are independently a bond, —N(R²³)—, —O—, —S—, —C(O)—, —N(R²³)C(O)—, —C(O)N(R²⁴)—, —N(R²³)C(O)N(R²⁴)—, —C(O)O—, —OC(O)—, —N(R²³)C(O)O—, —OC(O)N(R²⁴)—, —OPO₂—O—, —O—P(O)(S)—O—, —O—P(O)(R²⁵)—O—, —O—P(S)(R²⁵)—O—, —O—P(O)(NR²³R²⁴)—N—, —O—P(S)(NR²³R²⁴)—N—, —O—P(O)(NR²³R²⁴)—O—, —O—P(S)(NR²³R²⁴)—O—, —P(O)(NR²³R²⁴)—N—, —P(S)(NR²³R²⁴)—N—, —P(O)(NR²³R²⁴)—O—, —P(S)(NR²³R²⁴)—O—, —S—S—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene. Each R²³, R²⁴ and R²⁵ is independently hydrogen or unsubstituted C₁-C₁₀ alkyl.

L⁵ is -L^{5A}-L^{5B}-L^{5C}-L^{5D}-L^{5E}-. L⁶ is -L^{6A}-L^{6B}-L^{6C}-L^{6D}-L^{6E}-. L^{5A}, L^{5B}, L^{5C}, L^{5D}, L^{5E}, L^{6A}, L^{6B}, L^{6C}, L^{6D}, and L^{6E} are independently a

bond, —NH—, —O—, —S—, —C(O)—, —NHC(O)—, —NHC(O)NH—, —C(O)O—, —OC(O)—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene.

R¹ and R² are independently unsubstituted C₁-C₂₅ alkyl, wherein at least one of R¹ and R² is unsubstituted C₉-C₁₉ alkyl. R³ is

hydrogen, —NH₂, —OH, —SH, —C(O)H, —C(O)NH₂, —NHC(O)H, —NHC(O)OH, —NHC(O)NH₂, —C(O) OH, —OC(O)H, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In an aspect, provided is a method including contacting a cell with the compound, or the compound including a nucleic acid (A), as described herein.

In an aspect, provided is a method comprising administering to a subject the compound, or the compound including a nucleic acid (A), as described herein.

In an aspect, provided is a compound, or the compound including a nucleic acid (A) as described herein, for use in therapy

In an aspect, provided is a method of introducing a nucleic acid into a cell within a subject. The method includes administering to said subject the compound including a nucleic acid (A) as described herein.

In an aspect, provided is a cell including the compound including a nucleic acid (A) as described herein.

In an aspect, provided is a pharmaceutical composition including a pharmaceutically acceptable excipient and the compound including a nucleic acid (A) as described herein.

Other aspects are disclosed infra.

### BRIEF DESCRIPTION OF THE DRAWINGS

FIG. 1A shows a structure of DT-000137 according to an exemplary embodiment.

FIG. 1B shows a structure of DT-000146 according to an exemplary embodiment.

FIG. 1C shows a structure of DT-000347 according to an exemplary embodiment.

FIG. 1D shows a structure of DT-000155 according to an exemplary embodiment.

FIG. 1E shows a structure of DT-000156 according to an exemplary embodiment.

FIG. 1F shows a structure of DT-000157 according to an exemplary embodiment.

FIG. 1G shows a structure of DT-000272 according to an exemplary embodiment.

FIG. 1H shows a structure of DT-000273 according to an exemplary embodiment.

FIG. 1I shows a structure of DT-000274 according to an exemplary embodiment.

FIG. 1J shows a structure of DT-000275 according to an exemplary embodiment.

FIG. 1K shows a structure of DT-000276 according to an exemplary embodiment.

FIG. 1L shows a structure of DT-000277 according to an exemplary embodiment.

FIG. 1M shows a structure of DT-000278 according to an exemplary embodiment.

FIG. 1N shows a structure of DT-000350 according to an exemplary embodiment.

FIG. 1O shows a structure of DT-000183.

### DETAILED DESCRIPTION

### Definitions

Unless defined otherwise, all technical terms, scientific terms, abbreviations, chemical structures, and chemical formulae used herein have the same meaning as is commonly understood by one of ordinary skill in the art. The chemical structures and formulae set forth herein are constructed according to the standard rules of chemical valency known in the chemical arts. All patents, applications, published applications, and other publications referenced herein are incorporated by reference in their entirety unless stated otherwise. Unless otherwise indicated, conventional methods of mass spectroscopy, NMR, HPLC, protein chemistry, biochemistry, recombinant DNA techniques, and pharmacology are employed. Furthermore, use of the term “including” as well as other forms, such as “include”, “includes,” and “included,” is not limiting. As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least.” When used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbons (e.g., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated. An alkenyl may include more than one double bond and/or one or more triple bonds in addition to the one or more double bonds. An alkynyl may include more than one triple bond and/or one or more double bonds in addition to the one or more triple bonds.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems are bridged monocyclic rings or fused bicyclic rings. In embodiments, bridged monocyclic rings contain a monocyclic cycloalkyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. In embodiments, fused bicyclic cycloalkyl ring systems contain a monocyclic cycloalkyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkyl is attached to the parent molecular moiety through any carbon atom contained within the monocyclic cycloalkyl ring. In embodiments, cycloalkyl groups are optionally substituted with one or two groups which are independently oxo or thia. In embodiments, the fused bicyclic cycloalkyl is a 5 or 6 membered monocyclic cycloalkyl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused bicyclic cycloalkyl is optionally substituted by one or two groups which are independently oxo or thia. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkyl is attached to the parent molecular moiety through any carbon atom contained within the base ring. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic cycloalkyl groups include, but are not limited to tetradecahydrophenanthrenyl, perhydrophenothiazin-1-yl, and perhydrophenoxazin-1-yl.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. In embodiments, monocyclic cycloalkenyl ring systems are cyclic hydrocarbon groups containing from 3 to 8 carbon atoms, where such groups are unsaturated (i.e., containing at least one annular carbon carbon double bond), but not aromatic. Examples of monocyclic cycloalkenyl ring systems include cyclopentenyl and cyclohexenyl. In embodiments, bicyclic cycloalkenyl rings are bridged monocyclic rings or a fused bicyclic rings. In embodiments, bridged monocyclic rings contain a monocyclic cycloalkenyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic cycloalkenyls include, but are not limited to, norbornenyl and bicyclo[2.2.2]oct 2 enyl. In embodiments, fused bicyclic cycloalkenyl ring systems contain a monocyclic cycloalkenyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkenyl is attached to the parent molecular moiety through any carbon atom contained within the monocyclic cycloalkenyl ring. In embodiments, cycloalkenyl groups are optionally substituted with one or two groups which are independently oxo or thia. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkenyl is attached to the parent molecular moiety through any carbon atom contained within the base ring. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a monocyclic heteroaryl, a monocyclic cycloalkyl, a monocyclic cycloalkenyl, and a monocyclic heterocyclyl.

In embodiments, a heterocycloalkyl is a heterocyclyl. The term “heterocyclyl” as used herein, means a monocyclic, bicyclic, or multicyclic heterocycle. The heterocyclyl monocyclic heterocycle is a 3, 4, 5, 6 or 7 membered ring containing at least one heteroatom independently selected from the group consisting of O, N, and S where the ring is saturated or unsaturated, but not aromatic. The 3 or 4 membered ring contains 1 heteroatom selected from the group consisting of O, N and S. The 5 membered ring can contain zero or one double bond and one, two or three heteroatoms selected from the group consisting of O, N and S. The 6 or 7 membered ring contains zero, one or two double bonds and one, two or three heteroatoms selected from the group consisting of O, N and S. The heterocyclyl monocyclic heterocycle is connected to the parent molecular moiety through any carbon atom or any nitrogen atom contained within the heterocyclyl monocyclic heterocycle. Representative examples of heterocyclyl monocyclic heterocycles include, but are not limited to, azetidinyl, azepanyl, aziridinyl, diazepanyl, 1,3 dioxanyl, 1,3 dioxolanyl, 1,3 dithiolanyl, 1,3 dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1 dioxidothiomorpholinyl (thiomorpholine sulfone), thiopyranyl, and trithianyl. The heterocyclyl bicyclic heterocycle is a monocyclic heterocycle fused to either a phenyl, a monocyclic cycloalkyl, a monocyclic cycloalkenyl, a monocyclic heterocycle, or a monocyclic heteroaryl. The heterocyclyl bicyclic heterocycle is connected to the parent molecular moiety through any carbon atom or any nitrogen atom contained within the monocyclic heterocycle portion of the bicyclic ring system. Representative examples of bicyclic heterocyclyls include, but are not limited to, 2,3 dihydrobenzofuran 2 yl, 2,3 dihydrobenzofuran 3 yl, indolin 1 yl, indolin 2 yl, indolin 3 yl, 2,3 dihydrobenzothien 2 yl, decahydroquinolinyl, decahydroisoquinolinyl, octahydro 1H indolyl, and octahydrobenzofuranyl. In embodiments, heterocyclyl groups are optionally substituted with one or two groups which are independently oxo or thia. In certain embodiments, the bicyclic heterocyclyl is a 5 or 6 membered monocyclic heterocyclyl ring fused to a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the bicyclic heterocyclyl is optionally substituted by one or two groups which are independently oxo or thia. Multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. The multicyclic heterocyclyl is attached to the parent molecular moiety through any carbon atom or nitrogen atom contained within the base ring. In embodiments, multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic heterocyclyl groups include, but are not limited to 10H-phenothiazin-10-yl, 9,10-dihydroacridin-9-yl, 9,10-dihydroacridin-10-yl, 10H-phenoxazin-10-yl, 10,11-dihydro-5H-dibenzo[b,f]azepin-5-yl, 1,2,3,4-tetrahydropyrido[4,3-g]isoquinolin-2-yl, 12H-benzo[b]phenoxazin-12-yl, and dodecahydro-1H-carbazol-9-yl.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom (e.g., O, N, S, Si, or P), and wherein the nitrogen and sulfur atoms may optionally be oxidized, and the nitrogen heteroatom may optionally be quaternized. The heteroatom(s) (e.g., O, N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzooxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g. substituents for cycloalkyl or heterocycloalkyl rings). Spirocyclic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g. all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” denotes the point of attachment of a chemical moiety to the remainder of a molecule or chemical formula.

The term “oxo,” as used herein, means an oxygen that is double bonded to a carbon atom.

The term “alkylarylene” as an arylene moiety covalently bonded to an alkylene moiety (also referred to herein as an alkylene linker). In embodiments, the alkylarylene group has the formula:

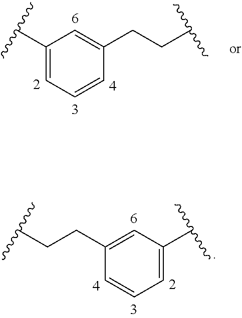 [SMILES: *CCc1cccc(*)c1 ; *CCc1cccc(*)c1]

An alkylarylene moiety may be substituted (e.g. with a substituent group) on the alkylene moiety or the arylene linker (e.g. at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃—SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″, —NR′C(O)—OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl, heteroalkyl, cycloalkyl, heterocycloalkyl, aryl, heteroaryl, substituted with at least one substituent selected from:
   (i) oxo, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (ii) alkyl, heteroalkyl, cycloalkyl, heterocycloalkyl, aryl, heteroaryl, substituted with at least one substituent selected from:
      (a) oxo, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
      (b) alkyl, heteroalkyl, cycloalkyl, heterocycloalkyl, aryl, heteroaryl, substituted with at least one substituent selected from: oxo, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

In embodiments of the compounds herein, each substituted or unsubstituted alkyl may be a substituted (e.g., substituted with a substituent group, a size-limited substituent group, or lower substituent group) or unsubstituted C₁-C₂₀ alkyl, each substituted or unsubstituted heteroalkyl is a substituted (e.g., substituted with a substituent group, a size-limited substituent group, or lower substituent group) or unsubstituted 2 to 20 membered heteroalkyl, each substituted or unsubstituted cycloalkyl is a substituted (e.g., substituted with a substituent group, a size-limited substituent group, or lower substituent group) or unsubstituted C₃-C₈ cycloalkyl, each substituted or unsubstituted heterocycloalkyl is a substituted (e.g., substituted with a substituent group, a size-limited substituent group, or lower substituent group) or unsubstituted 3 to 8 membered heterocycloalkyl, each or unsubstituted aryl is a substituted (e.g., substituted with a substituent group, a size-limited substituent group, or lower substituent group) or unsubstituted C₆-C₁₀ aryl, and/or each substituted or unsubstituted heteroaryl is a substituted (e.g., substituted with a substituent group, a size-limited substituent group, or lower substituent group) or unsubstituted 5 to 10 membered heteroaryl. In embodiments herein, each substituted or unsubstituted alkylene is a substituted (e.g., substituted with a substituent group, a size-limited substituent group, or lower substituent group) or unsubstituted C₁-C₂₀ alkylene, each substituted or unsubstituted heteroalkylene is a substituted (e.g., substituted with a substituent group, a size-limited substituent group, or lower substituent group) or unsubstituted 2 to 20 membered heteroalkylene, each substituted or unsubstituted cycloalkylene is a substituted (e.g., substituted with a substituent group, a size-limited substituent group, or lower substituent group) or unsubstituted C₃-C₈ cycloalkylene, each substituted or unsubstituted heterocycloalkylene is a substituted (e.g., substituted with a substituent group, a size-limited substituent group, or lower substituent group) or unsubstituted 3 to 8 membered heterocycloalkylene, each substituted or unsubstituted arylene is a substituted (e.g., substituted with a substituent group, a size-limited substituent group, or lower substituent group) or unsubstituted C₆-C₁₀ arylene, and/or each substituted or unsubstituted heteroarylene is a substituted (e.g., substituted with a substituent group, a size-limited substituent group, or lower substituent group) or unsubstituted 5 to 10 membered heteroarylene.

In embodiments, each substituted or unsubstituted alkyl is a substituted (e.g., substituted with a substituent group, a size-limited substituent group, or lower substituent group) or unsubstituted C₁-C₈ alkyl, each substituted or unsubstituted heteroalkyl is a substituted (e.g., substituted with a substituent group, a size-limited substituent group, or lower substituent group) or unsubstituted 2 to 8 membered heteroalkyl, each substituted or unsubstituted cycloalkyl is a substituted (e.g., substituted with a substituent group, a size-limited substituent group, or lower substituent group) or unsubstituted C₃-C₇ cycloalkyl, each substituted or unsubstituted heterocycloalkyl is a substituted (e.g., substituted with a substituent group, a size-limited substituent group, or lower substituent group) or unsubstituted 3 to 7 membered heterocycloalkyl, each substituted or unsubstituted aryl is a substituted (e.g., substituted with a substituent group, a size-limited substituent group, or lower substituent group) or unsubstituted C₆-C₁₀ aryl, and/or each substituted or unsubstituted heteroaryl is a substituted (e.g., substituted with a substituent group, a size-limited substituent group, or lower substituent group) or unsubstituted 5 to 9 membered heteroaryl. In embodiments, each substituted or unsubstituted alkylene is a substituted (e.g., substituted with a substituent group, a size-limited substituent group, or lower substituent group) or unsubstituted C₁-C₈ alkylene, each substituted or unsubstituted heteroalkylene is a substituted (e.g., substituted with a substituent group, a size-limited substituent group, or lower substituent group) or unsubstituted 2 to 8 membered heteroalkylene, each substituted or unsubstituted cycloalkylene is a substituted (e.g., substituted with a substituent group, a size-limited substituent group, or lower substituent group) or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted (e.g., substituted with a substituent group, a size-limited substituent group, or lower substituent group) or unsubstituted C₆-C₁₀ arylene, and/or each substituted or unsubstituted heteroarylene is a substituted (e.g., substituted with a substituent group, a size-limited substituent group, or lower substituent group) or unsubstituted 5 to 9 membered heteroarylene. In embodiments, the compound is a chemical species set forth in the Examples section, figures, or tables below.

Certain compounds provided herein possess asymmetric carbon atoms (optical or chiral centers) or double bonds; the enantiomers, racemates, diastereomers, tautomers, geometric isomers, stereoisomeric forms that may be defined, in terms of absolute stereochemistry, as (R)- or (S)- or, as (D)- or (L)- for amino acids, and individual isomers are encompassed within the scope of the present disclosure. The compounds of provided herein do not include those that are known in art to be too unstable to synthesize and/or isolate. Compounds provided herein include those in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds provided herein may exist in tautomeric forms, all such tautomeric forms of the compounds being within the scope of the present disclosure.

Where the compounds disclosed herein have at least one chiral center, they may exist as individual enantiomers and diastereomers or as mixtures of such isomers, including racemates. Separation of the individual isomers or selective synthesis of the individual isomers is accomplished by application of various methods which are well known to practitioners in the art. Unless otherwise indicated, all such isomers and mixtures thereof are included in the scope of the compounds disclosed herein. Unless otherwise stated, structures depicted herein are also meant to include all stereochemical forms of the structure; i.e., the (R) and (S) configurations for each asymmetric center. Therefore, single stereochemical isomers as well as enantiomeric and diastereomeric mixtures of the present compounds, generally recognized as stable by those skilled in the art, are within the scope of the present disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, replacement of fluoride by ¹⁸F, or the replacement of a carbon by ¹³C- or ¹⁴C-enriched carbon are within the scope of the present disclosure.

The compounds provided herein may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds provided herein, whether radioactive or not, are included within the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

“Analog,” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman decimal symbol may be used to distinguish each appearance of that particular R group. For example, where multiple R¹³ substituents are present, each R¹³ substituent may be distinguished as R^{13.1}, R^{13.2}, R^{13.3}, R^{13.4}, etc., wherein each of R^{13.1}, R^{13.2}, R^{13.3}, R^{13.4}, etc. is defined within the scope of the definition of R¹³ and optionally differently. The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Description of compounds of provided herein is limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “pharmaceutically acceptable salts” refers to salts that retain the biological effectiveness and properties of a compound, which are not biologically or otherwise undesirable for use in a pharmaceutical. In many cases, the compounds herein are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Many such salts are known in the art, as described in WO 87/05297, Johnston et al., published Sep. 11, 1987 (incorporated by reference herein in its entirety).

“Contacting” is used in accordance with its plain ordinary meaning and refers to the process of allowing at least two distinct species (e.g. chemical compounds, biomolecules or cells) to become sufficiently proximal to react, interact or physically touch. For example, contacting includes the process of allowing a compound to become sufficiently proximal to a cell to bind to a cell-surface receptor.

As used herein, “contacting a cell” refers to a condition in which a compound or other composition of matter is in direct contact with a cell, or is close enough to induce a desired biological effect in a cell.

As defined herein, the term “inhibition”, “inhibit”, “inhibiting” and the like mean negatively affecting (e.g. decreasing) activity or function relative to the activity or function in the absence of the inhibitor. In embodiments inhibition means negatively affecting (e.g. decreasing) the concentration or levels of a biomolecule, such as a protein or mRNA, relative to the concentration or level of the biomolecule in the absence of the inhibitor. For example, inhibition includes decreasing the level of mRNA expression in a cell. In embodiments, inhibition refers to a reduction in the activity of a particular biomolecule target, such as a protein target or an mRNA target. Thus, inhibition includes, at least in part, partially or totally blocking stimulation, decreasing, preventing, or delaying activation, or inactivating, desensitizing, or down-regulating signal transduction or enzymatic activity or the amount of a biomolecule. In embodiments, inhibition refers to a reduction of activity of a target biomolecule resulting from a direct interaction (e.g. an inhibitor binds to a target protein). In embodiments, inhibition refers to a reduction of activity of a target biomolecule from an indirect interaction (e.g. an inhibitor binds to a protein that activates a target protein, thereby preventing target protein activation).

The term “inhibitor” also refers to a compound, composition, or substance capable of detectably decreasing the expression or activity of a given gene or protein. For example, an inhibitor may decrease expression or activity 10%, 20%, 30%, 40%, 50%, 60%, 70%, 80%, 90% or more in comparison to a control in the absence of the inhibitor. Inhibitors include, for example, synthetic or biological molecules, such as oligonucleotides.

The terms “expression” and “gene expression” as used herein refer to the steps involved in the translation of a nucleic acid into a protein, including mRNA expression and protein expression. Expression can be detected using conventional techniques for detecting nucleic acids or proteins (e.g., PCR, ELISA, Southern blotting, Western blotting, flow cytometry, FISH, immunofluorescence, immunohistochemistry).

An “effective amount” is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist.

The term “in vivo” used herein means a process that takes place within a subject's body.

The term “subject” used herein means a human or non-human animal selected for treatment or therapy. In embodiments, a subject is a human.

The term “ex vivo” used herein means a process that takes place in vitro in isolated tissue or cells where the treated tissue or cells comprise primary cells. As is known in the art, any medium used in this process can be aqueous and non-toxic so as not to render the tissue or cells non-viable. In embodiments, the ex vivo process takes place in vitro using primary cells.

The term “administration” means providing a pharmaceutical agent or composition to a subject, and includes administration performed by a medical professional and self-administration.

The term “therapy” means the application of one or more specific procedures used for the amelioration of at least one indicator or a disease or condition. In embodiments, the specific procedure is the administration of one or more pharmaceutical agents.

The term “modulate” is used herein in its ordinary sense as understood by a person of ordinary skill in the art, and thus refers to the act of changing or varying one or more properties. For example, in the context of a modulator's effects on a target molecule, to modulate means to change by increasing or decreasing a property or function of the target molecule or the amount of the target molecule. A modulator of a disease decreases a symptom, cause, or characteristic of the targeted disease.

The terms “nucleic acid” means compounds containing at least two nucleotide monomers covalently linked together. Nucleic acids include polynucleotides and oligonucleotides, including double-stranded oligonucleotides and single-stranded oligonucleotides, and modified versions thereof.

The term “polynucleotide” means a longer length nucleic acid, e.g., 200, 300, 500, 1000, 2000, 3000, 5000, 7000, or 10,000 nucleotides in length. Non-limiting examples of polynucleotides include a gene, a gene fragment, an exon, an intron, intergenic DNA (including, without limitation, heterochromatic DNA), messenger RNA (mRNA), a long non-coding RNA, transfer RNA, ribosomal RNA, a ribozyme, cDNA, a recombinant polynucleotide, a branched polynucleotide, a plasmid, a vector, isolated DNA of a sequence, and an isolated RNA of a sequence. Polynucleotides useful in the methods of the disclosure may include natural nucleic acid sequences and variants thereof, artificial nucleic acid sequences, or a combination of such sequences.

The term“oligonucleotide” means a shorter length nucleic acid, e.g. of less than 100 nucleotides in length. Oligonucleotides may be single-stranded or double-stranded. An oligonucleotide may comprise naturally occurring ribonucleotides, naturally occurring deoxyribonucleotides, and/or nucleotides having one or more modifications to a naturally occurring terminus, sugar, nucleobase, and/or internucleotide linkage. Non-limiting examples of oligonucleotides include double-stranded oligonucleotides, single-stranded oligonucleotides, antisense oligonucleotides, small interfering RNA (siRNA), microRNA mimics, short hairpin RNAs (shRNA), single-strand small interfering RNA (ssRNAi), RNaseH oligonucleotides, anti-microRNA oligonucleotides, steric blocking oligonucleotides, exon-skipping oligonucleotides, CRISPR guide RNAs, and aptamers.

The term “double-stranded oligonucleotide” means an oligonucleotide that is substantially in a duplex form. Double-stranded oligonucleotides may comprise structures where the duplex region is formed between two anti-parallel oligonucleotides that are not covalently linked, as in an siRNA or microRNA mimic. Such double-stranded oligonucleotides may have a short nucleotide overhang at one or both ends of the duplex structure. Double-stranded oligonucleotides may also include a single oligonucleotide with sufficient length and self-complementarity to form a duplex structure, as in an shRNA. Such double-stranded oligonucleotides include stem-loop structures. A double-stranded nucleic acid may include one or more modifications relative to a naturally occurring terminus, sugar, nucleobase, and/or phosphate group. Non-limiting examples of double-stranded oligonucleotides include small interfering RNA (siRNA), short hairpin RNA (shRNA), and microRNA mimics.

The term “small interfering RNA” or “siRNA” means a double-stranded oligonucleotide formed from separate antisense and sense strands, which interferes with the expression of genes in a sequence-specific manner by facilitating mRNA degradation before translation through the RNA interference pathway. The antisense and sense strands of an siRNA are not covalently linked.

The term “microRNA mimic” means a synthetic version of a naturally occurring microRNA. A microRNA mimic comprises a antisense strand, which is complementary to one or more target mRNAs, and a sense strand which is complementary to the antisense strand. In naturally occurring microRNAs, the antisense strand is typically only partially complementary to its target mRNA(s), and the sense strand is only partially complementary to the antisense strand. A microRNA mimic may comprise nucleobase sequences having 100% identity to the naturally occurring microRNA or may comprise a nucleobase sequences less than 100% identical to the naturally occurring microRNA. For example, a microRNA mimic may comprise a sense strand that is 100% complementary to the antisense strand.

The term “single-strand RNA interfering” or “ssRNAi” means a single-stranded oligonucleotide which interferes with the expression of genes in a sequence-specific manner by facilitating mRNA degradation before translation through the RNA interference pathway.

The term “antisense strand” means an oligonucleotide of an siRNA or a ssRNAi that is complementary to the target mRNA and is incorporated into the RNA-induced silencing complex (RISC) to direct gene silencing in a sequence-specific manner through the RNA interference pathway. An antisense strand may also be referred to as the “guide strand.”

The term “sense strand” means an oligonucleotide that is complementary to the antisense strand of a double-stranded oligonucleotide. The sense strand is typically degraded following incorporation of the antisense strand into RISC. The sense strand may also be referred to as the “passenger strand.”

The term “duplex region” means a structure formed through nucleotide base-pairing of complementary oligonucleotide sequences. A duplex region may be formed from portions of complementary sequences, or from full lengths of complementary sequences.

The term “short hairpin RNA” or “shRNA” means a double-stranded oligonucleotide containing a loop structure that is processed in a cell to an siRNA which interferes with the expression of genes in a sequence-specific manner, by facilitating mRNA degradation before translation through the RNA interference pathway.

The term “nucleotide overhang” means contiguous single-stranded nucleotides at the end of an oligonucleotide in a double-stranded oligonucleotide.

The term “single-stranded oligonucleotide” means an oligonucleotide that is not hybridized to a complementary strand. Non-limiting examples of single-stranded oligonucleotides include single-strand small interfering RNA (ssRNAi), RNaseH oligonucleotides (oligonucleotides chemically modified to elicit RNaseH-mediated degradation of a target RNA), anti-microRNA oligonucleotides (oligonucleotides complementary to microRNAs), steric blocking oligonucleotides (oligonucleotides that interfere with target RNA activity without degrading the target RNA), exon-skipping oligonucleotides (oligonucleotides that hybridized to an exon annealing site and alter splicing), CRISPR guide RNAs, and aptamers.

The term “hybridize” means the annealing of one nucleic acid to another nucleic acid based on nucleobase sequence complementarity. In embodiments, an antisense strand is hybridized to a sense strand. In embodiments, an antisense strand hybridizes to a target mRNA sequence.

The term “complementary” means nucleobases having the capacity to pair non-covalently via hydrogen bonding.

The term “fully complementary” means each nucleobase of a first nucleic acid is complementary to each nucleobase of a second nucleic acid. In embodiments, an antisense strand is fully complementary to its target mRNA. In embodiments, a sense strand and an antisense strand of a double-stranded oligonucleotide are fully complementary over their entire lengths. In embodiments, a sense strand and an antisense strand of double-stranded oligonucleotide are fully complementary over the entire length of the double-stranded region of the siRNA, and one or both termini of either strand comprises single-stranded nucleotides.

The term “nucleoside” means a monomer of a nucleobase and a pentofuranosyl sugar (e.g., either ribose or deoxyribose). Nucleosides may be modified at the nucleobase and/or and the sugar. In embodiments, a nucleoside is a deoxyribonucleoside. In embodiments, a nucleoside is a ribonucleoside.

The term “nucleotide” means a nucleoside covalently linked to a phosphate group at the 5′-carbon of the pentafuranosyl sugar. Nucleotides may be modified at one or more of the nucleobase, sugar, or phosphate group. A nucleotide may have a ligand attached, either directly or through a linker. In embodiments, a nucleotide is a deoxyribonucleotide. In embodiments, a nucleotide is a ribonucleotide.

The term “nucleobase” means the heterocyclic base moiety of a nucleoside or nucleotide. Non-limiting examples of nucleobases includes cytosine or a derivative thereof (e.g., cytosine analogue), guanine or a derivative thereof (e.g., guanine analogue), adenine or a derivative thereof (e.g., adenine analogue), thymine or a derivative thereof (e.g., thymine analogue), uracil or a derivative thereof (e.g., uracil analogue), hypoxanthine or a derivative thereof (e.g., hypoxanthine analogue), xanthine or a derivative thereof (e.g., xanthine analogue), 7-methylguanine or a derivative thereof (e.g., 7-methylguanine analogue), deaza-adenine or a derivative thereof (e.g., deaza-adenine analogue), deaza-guanine or a derivative thereof (e.g., deaza-guanine), deaza-hypoxanthine or a derivative thereof, 5,6-dihydrouracil or a derivative thereof (e.g., 5,6-dihydrouracil analogue), 5-methylcytosine or a derivative thereof (e.g., 5-methylcytosine analogue), or 5-hydroxymethylcytosine or a derivative thereof (e.g., 5-hydroxymethylcytosine analogue) moieties. In embodiments, the nucleobase is adenine, guanine, hypoxanthine, xanthine, theobromine, caffeine, uric acid, or isoguanine, which may be optionally substituted or modified. In embodiments, the nucleobase is

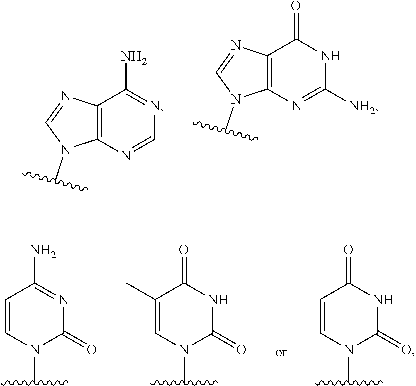

which may be optionally substituted or modified.

The term “modified nucleotide” means a nucleotide having one or more modifications relative to a naturally occurring nucleotide. A modification may be present in an internucleoside linkage, a nucleobase, and/or a sugar moiety of the nucleotide. A modified nucleotide may be selected over an unmodified form because of desirable properties such as, for example, enhanced cellular uptake, enhanced affinity for other oligonucleotides or nucleic acid targets, increased stability in the presence of nucleases, and/or reduced immune stimulation. A modified nucleotide may have a modified sugar moiety and an unmodified phosphate group. A modified nucleotide may have an unmodified sugar moiety and a modified phosphate group. A modified nucleotide may have a modified sugar moiety and an unmodified nucleobase. A modified nucleotide may have a modified sugar moiety and a modified phosphate group. Nucleic acids, polynucleotides and oligonucleotides may comprise one or more modified nucleotides.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that participate in nucleobase-pairing (i.e., about 60% complementarity, preferably 65%, 70%, 75%, 80%, 85%, 90%, 91%, 92%, 93%, 94%, 95%, 96%, 97%, 98%, 99%, or higher complementarity over a specified region).

“Hybridize” shall mean the annealing of one single-stranded nucleic acid (such as a primer) to another nucleic acid based on the well-understood principle of sequence complementarity. In an embodiment the other nucleic acid is a single-stranded nucleic acid. The propensity for hybridization between nucleic acids depends on the temperature and ionic strength of their miliu, the length of the nucleic acids and the degree of complementarity. The effect of these parameters on hybridization is described in, for example, Sambrook J, Fritsch E F, Maniatis T., Molecular cloning: a laboratory manual, Cold Spring Harbor Laboratory Press, New York (1989). As used herein, hybridization of a primer, or of a DNA extension product, respectively, is extendable by creation of a phosphodiester bond with an available nucleotide or nucleotide analogue capable of forming a phosphodiester bond, therewith.

The terms “identical” or percent “identity,” in the context of two or more nucleic acids or polypeptide sequences, refer to two or more sequences or subsequences that are the same or have a specified percentage of amino acid residues or nucleotides that are the same (i.e., at least 60% identity, or at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or within a range defined by any of two of the preceding values, identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). This definition also refers to, or may be applied to, the complement of a test sequence. The definition also includes sequences that have deletions and/or additions, as well as those that have substitutions. As described below, the preferred algorithms can account for gaps, insertions and the like. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

### Compounds

Provided are, inter alia, compounds including a nucleic acid (A) that is covalently bonded to one or more half-life extension motifs (HLEMs). For example, the compound includes a nucleic acid (A) covalently bonded to one half-life extension motif (HLEM), two half-life extension motifs (HLEMs), three half-life extension motifs (HLEMs), four half-life extension motifs (HLEMs), or five half-life extension motifs (HLEMs).

In an aspect, the compound has a formula (I)

(HLEM)_{z}-A (I),

wherein z is an integer from 1 to 5.

In embodiments, z is 1. In embodiments, z is 2. In embodiments, z is 3. In embodiments, z is 4. In embodiments, z is 5.

In an aspect, the nucleic acid is covalently bonded to one or more uptake motifs (UMs). For example, the nucleic acid is covalently bonded to one uptake motif (UM), two uptake motifs (UMs), three uptake motifs (UMs), four uptake motifs (UMs), or five uptake motifs (UMs).

In an aspect, the compound has a formula (II):

(HLEM)_{z}-A-(UM)ₜ (II),

wherein t is an integer from 1 to 5.

In embodiments, t is 1. In embodiments, t is 2. In embodiments, t is 3. In embodiments, t is 4. In embodiments, t is 5.

In embodiments, the half-life extension motif has the structure:

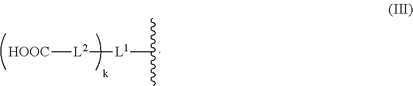

k is an integer from 1 to 5.

L¹ is independently a covalent linker. L² is independently an unsubstituted alkylene.

In embodiments, k is 1. In embodiments, k is 2. In embodiments, k is 3. In embodiments, k is 4. In embodiments, k is 5. In embodiments, k is an integer from 1 to 3. In embodiments, k is an integer from 1 to 2.

In embodiments, one or more L² may be attached to one or more atoms in L¹. In embodiments, one or more L² may be attached to one or more atoms in L¹ and the one or more atoms may be the same or different. In embodiments, the one or more L² are attached to the same atom. In embodiments, the one or more L² are attached to the different atoms. In embodiments, the one or more L² are attached to the same or different atoms.

In embodiments, one or more L² may be independently attached to L^{1A}, L^{1B}, L^{1C}, L^{1D}, or L^{1E}. In embodiments, L² may be independently attached to L^{1A}. In embodiments, one L² may be independently attached to L^{1B}. In embodiments, one L² may be independently attached to L^{1C}. In embodiments, one L² may be independently attached to L^{1D}. In embodiments, one L² may be independently attached to L^{1E}.

In embodiments, L^{1A}, L^{1B}, L^{1C}, L^{1D}, and L^{1E} are independently a bond, —N(R²⁰)—, —O—, —S—, —C(O)—, —N(R²⁰)C(O)—, —C(O)N(R²¹)—, —N(R²⁰)C(O)N(R²¹)—, —C(O)O—, —OC(O)—, —N(R²⁰)C(O)O—, —OC(O)N(R²¹)—, —OPO₂—O—, —O—P(O)(S)—O—, —O—P(O)(R²²)—O—, —O—P(S)(R²²)—O—, —O—P(O)(NR²⁰R²¹)—N—, —O—P(S)(NR²⁰R²¹)—N—, —O—P(O)(NR²⁰R²¹)—O—, —O—P(S)(NR²⁰R²¹)—O—, —P(O)(NR²⁰R²¹)—N—, —P(S)(NR²⁰R²¹)—N—, —P(O)(NR²⁰R²¹)—O—, —P(S)(NR²⁰R²¹)—O—, —S—S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. Each R²⁰, R²¹ and R²² is independently hydrogen or unsubstituted C₁-C₁₀ alkyl.

In embodiments, one or more L² may be independently attached to L^{1A}, L^{1B}, L^{1C}, L^{1D} or L^{1E}. In embodiments, one or more L² may be independently attached to L^{1A}. In embodiments, one or more L² may be independently attached to L^{1B}. In embodiments, one or more L² may be independently attached to L^{1C}. In embodiments, one or more L² may be independently attached to L^{1D}. In embodiments, one or more L² may be independently attached to L^{1E}.

In embodiments, at least one L² may be independently attached to L^{1A}. In embodiments, at least one L² may be independently attached to L^{1B}. In embodiments, at least one L² may be independently attached to L^{1C}. In embodiments, at least one L² may be independently attached to L^{1D}. In embodiments, at least one L² may be independently attached to L^{1E}.

In embodiments, one L² may be independently attached to L^{1A}. In embodiments, one L² may be independently attached to L^{1B}. In embodiments, one L² may be independently attached to L^{1C}. In embodiments, one L² may be independently attached to L^{1D}. In embodiments, one L² may be independently attached to L^{1E}.

In embodiments, L^{1A} is a bond, —N(R²⁰)—, —O—, —S—, —C(O)—, —N(R²⁰)C(O)—, —C(O)N(R²¹)—, —N(R²⁰)C(O)N(R²¹)—, —C(O)O—, —OC(O)—, —N(R²⁰)C(O)O—, —OC(O)N(R²¹)—, —OPO₂—O—, —O—P(O)(S)—O—, —O—P(O)(R²²)—O—, —O—P(S)(R²²)—O—, —O—P(O)(NR²⁰R²¹)—N—, —O—P(S)(NR²⁰R²¹)—N—, —O—P(O)(NR²⁰R²¹)—O—, —O—P(S)(NR²⁰R²¹)—O—, —P(O)(NR²⁰R²¹)—N—, —P(S)(NR²⁰R²¹)—N—, —P(O)(NR²⁰R²¹)—O—, —P(S)(NR²⁰R²¹)—O—, —S—S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, L^{1A} is a bond. In embodiments, L^{1A} is —N(R²⁰)—. In embodiments, L^{1A} is —O— or —S—. In embodiments, L^{1A} is —C(O)—. In embodiments, L^{1A} is —N(R²⁰)C(O)— or —C(O)N(R²¹)—. In embodiments, L^{1A} is —N(R²⁰)C(O)N(R²¹)—. In embodiments, L^{1A} is —C(O)O— or —OC(O)—. In embodiments, L^{1A} is —N(R²⁰)C(O)O— or —OC(O)N(R²¹)—. In embodiments, L^{1A} is —OPO₂—O—, —O—P(O)(S)—O—, —O—P(O)(R²²)—O—, —O—P(O)(NR²⁰R²¹)—N—, or O—P(O)(NR²⁰R²¹)—O—. In embodiments, L^{1A} is —P(O)(NR²⁰R²¹)—N—, —P(S)(NR²⁰R²¹)—N—, —P(O)(NR²⁰R²¹)—O— or —P(S)(NR²⁰R²¹)—O—. In embodiments, L^{1A} is —S—S—.

In embodiments, L^{1A} is independently substituted or unsubstituted alkylene (e.g., C₁-C₂₀, C₁-C₁₂, C₁-C₈, C₁-C₆, C₁-C₄, or C₁-C₂). In embodiments, L^{1A} is independently substituted alkylene (e.g., C₁-C₂₀, C₁-C₁₂, C₁-C₈, C₁-C₆, C₁-C₄, or C₁-C₂). In embodiments, L^{1A} is independently unsubstituted alkylene (e.g., C₁-C₂₀, C₁-C₁₂, C₁-C₈, C₁-C₆, C₁-C₄, or C₁-C₂). In embodiments, L^{1A} is independently substituted or unsubstituted C₁-C₂₀ alkylene. In embodiments, L^{1A} is independently substituted C₁-C₂₀ alkylene. In embodiments, L^{1A} is independently unsubstituted C₁-C₂₀ alkylene. In embodiments, L^{1A} is independently substituted or unsubstituted C₁-C₁₂ alkylene. In embodiments, L^{1A} is independently substituted C₁-C₁₂ alkylene. In embodiments, L^{1A} is independently unsubstituted C₁-C₁₂ alkylene. In embodiments, L^{1A} is independently substituted or unsubstituted C₁-C₈ alkylene. In embodiments, L^{1A} is independently substituted C₁-C₈ alkylene. In embodiments, L^{1A} is independently unsubstituted C₁-C₈ alkylene. In embodiments, L^{1A} is independently substituted or unsubstituted C₁-C₆ alkylene. In embodiments, L^{1A} is independently substituted C₁-C₆ alkylene. In embodiments, L^{1A} is independently unsubstituted C₁-C₆ alkylene. In embodiments, L^{1A} is independently substituted or unsubstituted C₁-C₄ alkylene. In embodiments, L^{1A} is independently substituted C₁-C₄ alkylene. In embodiments, L^{1A} is independently unsubstituted C₁-C₄ alkylene. In embodiments, L^{1A} is independently substituted or unsubstituted ethylene. In embodiments, L^{1A} is independently substituted ethylene. In embodiments, L^{1A} is independently unsubstituted ethylene. In embodiments, L^{1A} is independently substituted or unsubstituted methylene. In embodiments, L^{1A} is independently substituted methylene. In embodiments, L^{1A} is independently unsubstituted methylene.

In embodiments, L^{1A} is independently substituted or unsubstituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L^{1A} is independently substituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L^{1A} is independently unsubstituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L^{1A} is independently substituted or unsubstituted 2 to 20 membered heteroalkylene. In embodiments, L^{1A} is independently substituted 2 to 20 membered heteroalkylene. In embodiments, L^{1A} is independently unsubstituted 2 to 20 membered heteroalkylene. In embodiments, L^{1A} is independently substituted or unsubstituted 2 to 8 membered heteroalkylene. In embodiments, L^{1A} is independently substituted 2 to 8 membered heteroalkylene. In embodiments, L^{1A} is independently unsubstituted 2 to 8 membered heteroalkylene. In embodiments, L^{1A} is independently substituted or unsubstituted 2 to 6 membered heteroalkylene. In embodiments, L^{1A} is independently substituted 2 to 6 membered heteroalkylene. In embodiments, L^{1A} is independently unsubstituted 2 to 6 membered heteroalkylene. In embodiments, L^{1A} is independently substituted or unsubstituted 4 to 6 membered heteroalkylene. In embodiments, L^{1A} is independently substituted 4 to 6 membered heteroalkylene. In embodiments, L^{1A} is independently unsubstituted 4 to 6 membered heteroalkylene. In embodiments, L^{1A} is independently substituted or unsubstituted 2 to 3 membered heteroalkylene. In embodiments, L^{1A} is independently substituted 2 to 3 membered heteroalkylene. In embodiments, L^{1A} is independently unsubstituted 2 to 3 membered heteroalkylene. In embodiments, L^{1A} is independently substituted or unsubstituted 4 to 5 membered heteroalkylene. In embodiments, L^{1A} is independently substituted 4 to 5 membered heteroalkylene. In embodiments, L^{1A} is independently unsubstituted 4 to 5 membered heteroalkylene.

In embodiments, L^{1B} is a bond, —N(R²⁰)—, —O—, —S—, —C(O)—, —N(R²⁰)C(O)—, —C(O)N(R²¹)—, —N(R²⁰)C(O)N(R²¹)—, —C(O)O—, —OC(O)—, —N(R²⁰)C(O)O—, —OC(O)N(R²¹)—, —OPO₂—O—, —O—P(O)(S)—O—, —O—P(O)(R²²)—O—, —O—P(S)(R²²)—O—, —O—P(O)(NR²⁰R²¹)—N—, —O—P(S)(NR²⁰R²¹)—N—, —O—P(O)(NR²⁰R²¹)—O—, —O—P(S)(NR²⁰R²¹)—O—, —P(O)(NR²⁰R²¹)—N—, —P(S)(NR²⁰R²¹)—N—, —P(O)(NR²⁰R²¹)—O—, —P(S)(NR²⁰R²¹)—O—, —S—S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, L^{1B} is a bond. In embodiments, L^{1B} is —N(R²⁰)—. In embodiments, L^{1B} is —O— or —S—. In embodiments, L^{1B} is —C(O)—. In embodiments, L^{1B} is —N(R²⁰)C(O)— or —C(O)N(R²¹)—. In embodiments, L^{1B} is —N(R²⁰)C(O)N(R²¹)—. In embodiments, L^{1B} is —C(O)O— or —OC(O)—. In embodiments, L^{1B} is —N(R²⁰)C(O)O— or —OC(O)N(R²¹)—. In embodiments, L^{1B} is —PO₂—O—, —O—P(O)(S)—O—, —O—P(O)(R²²)—O—, —O—P(O)(NR²⁰R²¹)—N—, or O—P(O)(NR²⁰R²¹)—O—. In embodiments, L^{1B} is —P(O)(NR²⁰R²¹)—N—, —P(S)(NR²⁰R²¹)—N—, —P(O)(NR²⁰R²¹)—O— or —P(S)(NR²⁰R²¹)—O—. In embodiments, L^{1B} is —S—S—.

In embodiments, L^{1B} is independently substituted or unsubstituted alkylene (e.g., C₁-C₂₀, C₁-C₁₂, C₁-C₈, C₁-C₆, C₁-C₄, or C₁-C₂). In embodiments, L^{1B} is independently substituted alkylene (e.g., C₁-C₂₀, C₁-C₁₂, C₁-C₈, C₁-C₆, C₁-C₄, or C₁-C₂). In embodiments, L^{1B} is independently unsubstituted alkylene (e.g., C₁-C₂₀, C₁-C₁₂, C₁-C₈, C₁-C₆, C₁-C₄, or C₁-C₂). In embodiments, L^{1B} is independently substituted or unsubstituted C₁-C₂₀ alkylene. In embodiments, L^{1B} is independently substituted C₁-C₂₀ alkylene. In embodiments, L^{1B} is independently unsubstituted C₁-C₂₀ alkylene. In embodiments, L^{1B} is independently substituted or unsubstituted C₁-C₁₂ alkylene. In embodiments, L^{1B} is independently substituted C₁-C₁₂ alkylene. In embodiments, L^{1B} is independently unsubstituted C₁-C₁₂ alkylene. In embodiments, L^{1B} is independently substituted or unsubstituted C₁-C₈ alkylene. In embodiments, L^{1B} is independently substituted C₁-C₈ alkylene. In embodiments, L^{1B} is independently unsubstituted C₁-C₈ alkylene. In embodiments, L^{1B} is independently substituted or unsubstituted C₁-C₆ alkylene. In embodiments, L^{1B} is independently substituted C₁-C₆ alkylene. In embodiments, L^{1B} is independently unsubstituted C₁-C₆ alkylene. In embodiments, L^{1B} is independently substituted or unsubstituted C₁-C₄ alkylene. In embodiments, L^{1B} is independently substituted C₁-C₄ alkylene. In embodiments, L^{1B} is independently unsubstituted C₁-C₄ alkylene. In embodiments, L^{1B} is independently substituted or unsubstituted ethylene. In embodiments, L^{1B} is independently substituted ethylene. In embodiments, L^{1B} is independently unsubstituted ethylene. In embodiments, L^{1B} is independently substituted or unsubstituted methylene. In embodiments, L^{1B} is independently substituted methylene. In embodiments, L^{1B} is independently unsubstituted methylene.

In embodiments, L^{1B} is independently substituted or unsubstituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L^{1B} is independently substituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L^{1B} is independently unsubstituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L^{1B} is independently substituted or unsubstituted 2 to 20 membered heteroalkylene. In embodiments, L^{1B} is independently substituted 2 to 20 membered heteroalkylene. In embodiments, L^{1B} is independently unsubstituted 2 to 20 membered heteroalkylene. In embodiments, L^{1B} is independently substituted or unsubstituted 2 to 8 membered heteroalkylene. In embodiments, L^{1B} is independently substituted 2 to 8 membered heteroalkylene. In embodiments, L^{1B} is independently unsubstituted 2 to 8 membered heteroalkylene. In embodiments, L^{1B} is independently substituted or unsubstituted 2 to 6 membered heteroalkylene. In embodiments, L^{1B} is independently substituted 2 to 6 membered heteroalkylene. In embodiments, L^{1B} is independently unsubstituted 2 to 6 membered heteroalkylene. In embodiments, L^{1B} is independently substituted or unsubstituted 4 to 6 membered heteroalkylene. In embodiments, L^{1B} is independently substituted 4 to 6 membered heteroalkylene. In embodiments, L^{1B} is independently unsubstituted 4 to 6 membered heteroalkylene. In embodiments, L^{1B} is independently substituted or unsubstituted 2 to 3 membered heteroalkylene. In embodiments, L^{1B} is independently substituted 2 to 3 membered heteroalkylene. In embodiments, L^{1B} is independently unsubstituted 2 to 3 membered heteroalkylene. In embodiments, L^{1B} is independently substituted or unsubstituted 4 to 5 membered heteroalkylene. In embodiments, L^{1B} is independently substituted 4 to 5 membered heteroalkylene. In embodiments, L^{1B} is independently unsubstituted 4 to 5 membered heteroalkylene.

In embodiments, L^{1C} is a bond, —N(R²⁰)—, —O—, —S—, —C(O)—, —N(R²⁰)C(O)—, —C(O)N(R²¹)—, —N(R²⁰)C(O)N(R²¹)—, —C(O)O—, —OC(O)—, —N(R²⁰)C(O)O—, —OC(O)N(R²¹)—, —OPO₂—O—, —O—P(O)(S)—O—, —O—P(O)(R²²)—O—, —O—P(S)(R²²)—O—, —O—P(O)(NR²⁰R²¹)—N—, —O—P(S)(NR²⁰R²¹)—N—, —O—P(O)(NR²⁰R²¹)—O—, —O—P(S)(NR²⁰R²¹)—O—, —P(O)(NR²⁰R²¹)—N—, —P(S)(NR²⁰R²¹)—N—, —P(O)(NR²⁰R²¹)—O—, —P(S)(NR²⁰R²¹)—O—, —S—S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, L^{1C} is a bond. In embodiments, L^{1C} is —N(R²⁰)—. In embodiments, L^{1C} is —O— or —S—. In embodiments, L^{1C} is —C(O)—. In embodiments, L^{1C} is —N(R²⁰)C(O)— or —C(O)N(R²¹)—. In embodiments, L^{1C} is —N(R²⁰)C(O)N(R²¹)—. In embodiments, L^{1C} is —C(O)O— or —OC(O)—. In embodiments, L^{1C} is —N(R²⁰)C(O)O— or —OC(O)N(R²¹)—. In embodiments, L^{1C} is —OPO₂—O—, —O—P(O)(S)—O—, —O—P(O)(R²²)—O—, —O—P(O)(NR²⁰R²¹)—N—, or O—P(O)(NR²⁰R²¹)—O—. In embodiments, L^{1C} is —P(O)(NR²⁰R²¹)—N—, —P(S)(NR²⁰R²¹)—N—, —P(O)(NR²⁰R²¹)—O— or —P(S)(NR²⁰R²¹)—O—. In embodiments, L^{1C} is —S—S—.

In embodiments, L^{1C} is independently substituted or unsubstituted alkylene (e.g., C₁-C₂₀, C₁-C₁₂, C₁-C₈, C₁-C₆, C₁-C₄, or C₁-C₂). In embodiments, L^{1C} is independently substituted alkylene (e.g., C₁-C₂₀, C₁-C₁₂, C₁-C₈, C₁-C₆, C₁-C₄, or C₁-C₂). In embodiments, L^{1C} is independently unsubstituted alkylene (e.g., C₁-C₂₀, C₁-C₁₂, C₁-C₈, C₁-C₆, C₁-C₄, or C₁-C₂). In embodiments, L^{1C} is independently substituted or unsubstituted C₁-C₂₀ alkylene. In embodiments, L^{1C} is independently substituted C₁-C₂₀ alkylene. In embodiments, L^{1C} is independently unsubstituted C₁-C₂₀ alkylene. In embodiments, L^{1C} is independently substituted or unsubstituted C₁-C₁₂ alkylene. In embodiments, L^{1C} is independently substituted C₁-C₁₂ alkylene. In embodiments, L^{1C} is independently unsubstituted C₁-C₁₂ alkylene. In embodiments, L^{1C} is independently substituted or unsubstituted C₁-C₈ alkylene. In embodiments, L^{1C} is independently substituted C₁-C₈ alkylene. In embodiments, L^{1C} is independently unsubstituted C₁-C₈ alkylene. In embodiments, L^{1C} is independently substituted or unsubstituted C₁-C₆ alkylene. In embodiments, L^{1C} is independently substituted C₁-C₆ alkylene. In embodiments, L^{1C} is independently unsubstituted C₁-C₆ alkylene. In embodiments, L^{1C} is independently substituted or unsubstituted C₁-C₄ alkylene. In embodiments, L^{1C} is independently substituted C₁-C₄ alkylene. In embodiments, L^{1C} is independently unsubstituted C₁-C₄ alkylene. In embodiments, L^{1C} is independently substituted or unsubstituted ethylene. In embodiments, L^{1C} is independently substituted ethylene. In embodiments, L^{1C} is independently unsubstituted ethylene. In embodiments, L^{1C} is independently substituted or unsubstituted methylene. In embodiments, L^{1C} is independently substituted methylene. In embodiments, L^{1C} is independently unsubstituted methylene.

In embodiments, L^{1C} is independently substituted or unsubstituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L^{1C} is independently substituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L^{1C} is independently unsubstituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L^{1C} is independently substituted or unsubstituted 2 to 20 membered heteroalkylene. In embodiments, L^{1C} is independently substituted 2 to 20 membered heteroalkylene. In embodiments, L^{1C} is independently unsubstituted 2 to 20 membered heteroalkylene. In embodiments, L^{1C} is independently substituted or unsubstituted 2 to 8 membered heteroalkylene. In embodiments, L^{1C} is independently substituted 2 to 8 membered heteroalkylene. In embodiments, L^{1C} is independently unsubstituted 2 to 8 membered heteroalkylene. In embodiments, L^{1C} is independently substituted or unsubstituted 2 to 6 membered heteroalkylene. In embodiments, L^{1C} is independently substituted 2 to 6 membered heteroalkylene. In embodiments, L^{1C} is independently unsubstituted 2 to 6 membered heteroalkylene. In embodiments, L^{1C} is independently substituted or unsubstituted 4 to 6 membered heteroalkylene. In embodiments, L^{1C} is independently substituted 4 to 6 membered heteroalkylene. In embodiments, L^{1C} is independently unsubstituted 4 to 6 membered heteroalkylene. In embodiments, L^{1C} is independently substituted or unsubstituted 2 to 3 membered heteroalkylene. In embodiments, L^{1C} is independently substituted 2 to 3 membered heteroalkylene. In embodiments, L^{1C} is independently unsubstituted 2 to 3 membered heteroalkylene. In embodiments, L^{1C} is independently substituted or unsubstituted 4 to 5 membered heteroalkylene. In embodiments, L^{1C} is independently substituted 4 to 5 membered heteroalkylene. In embodiments, L^{1C} is independently unsubstituted 4 to 5 membered heteroalkylene.

In embodiments, L^{1D} is a bond, —N(R²⁰)—, —O—, —S—, —C(O)—, —N(R²⁰)C(O)—, —C(O)N(R²¹)—, —N(R²⁰)C(O)N(R²¹)—, —C(O)O—, —OC(O)—, —N(R²⁰)C(O)O—, —OC(O)N(R²¹)—, —OPO₂—O—, —O—P(O)(S)—O—, —O—P(O)(R²²)—O—, —O—P(S)(R²²)—O—, —O—P(O)(NR²⁰R²¹)—N—, —O—P(S)(NR²⁰R²¹)—N—, —O—P(O)(NR²⁰R²¹)—O—, —O—P(S)(NR²⁰R²¹)—O—, —P(O)(NR²⁰R²¹)—N—, —P(S)(NR²⁰R²¹)—N—, —P(O)(NR²⁰R²¹)—O—, —P(S)(NR²⁰R²¹)—O—, —S—S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, L^{1D} is a bond. In embodiments, L^{1D} is —N(R²⁰)—. In embodiments, L^{1D} is —O— or —S—. In embodiments, L^{1D} is —C(O)—. In embodiments, L^{1D} is —N(R²⁰)C(O)— or —C(O)N(R²¹)—. In embodiments, L^{1D} is —N(R²⁰)C(O)N(R²¹)—. In embodiments, L^{1D} is —C(O)O— or —OC(O)—. In embodiments, L^{1D} is —N(R²⁰)C(O)O— or —OC(O)N(R²¹)—. In embodiments, L^{1D} is —PO₂—O—, —O—P(O)(S)—O—, —O—P(O)(R²²)—O—, —O—P(O)(NR²⁰R²¹)—N—, or O—P(O)(NR²⁰R²¹)—O—. In embodiments, L^{1D} is —P(O)(NR²⁰R²¹)—N—, —P(S)(NR²⁰R²¹)—N—, —P(O)(NR²⁰R²¹)—O— or —P(S)(NR²⁰R²¹)—O—. In embodiments, L^{1D} is —S—S—.

In embodiments, L^{1D} is independently substituted or unsubstituted alkylene (e.g., C₁-C₂₀, C₁-C₁₂, C₁-C₈, C₁-C₆, C₁-C₄, or C₁-C₂). In embodiments, L^{1D} is independently substituted alkylene (e.g., C₁-C₂₀, C₁-C₁₂, C₁-C₈, C₁-C₆, C₁-C₄, or C₁-C₂). In embodiments, L^{1D} is independently unsubstituted alkylene (e.g., C₁-C₂₀, C₁-C₁₂, C₁-C₈, C₁-C₆, C₁-C₄, or C₁-C₂). In embodiments, L^{1D} is independently substituted or unsubstituted C₁-C₂₀ alkylene. In embodiments, L^{1D} is independently substituted C₁-C₂₀ alkylene. In embodiments, L^{1D} is independently unsubstituted C₁-C₂₀ alkylene. In embodiments, L^{1D} is independently substituted or unsubstituted C₁-C₁₂ alkylene. In embodiments, L^{1D} is independently substituted C₁-C₁₂ alkylene. In embodiments, L^{1D} is independently unsubstituted C₁-C₁₂ alkylene. In embodiments, L^{1D} is independently substituted or unsubstituted C₁-C₈ alkylene. In embodiments, L^{1D} is independently substituted C₁-C₈ alkylene. In embodiments, L^{1D} is independently unsubstituted C₁-C₈ alkylene. In embodiments, L^{1D} is independently substituted or unsubstituted C₁-C₆ alkylene. In embodiments, L^{1D} is independently substituted C₁-C₆ alkylene. In embodiments, L^{1D} is independently unsubstituted C₁-C₆ alkylene. In embodiments, L^{1D} is independently substituted or unsubstituted C₁-C₄ alkylene. In embodiments, L^{1D} is independently substituted C₁-C₄ alkylene. In embodiments, L^{1D} is independently unsubstituted C₁-C₄alkylene. In embodiments, L^{1D} is independently substituted or unsubstituted ethylene. In embodiments, L^{1D} is independently substituted ethylene. In embodiments, L^{1D} is independently unsubstituted ethylene. In embodiments, L^{1D} is independently substituted or unsubstituted methylene. In embodiments, L^{1D} is independently substituted methylene. In embodiments, L^{1D} is independently unsubstituted methylene.

In embodiments, L^{1D} is independently substituted or unsubstituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L^{1D} is independently substituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L^{1D} is independently unsubstituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L^{1D} is independently substituted or unsubstituted 2 to 20 membered heteroalkylene. In embodiments, L^{1D} is independently substituted 2 to 20 membered heteroalkylene. In embodiments, L^{1D} is independently unsubstituted 2 to 20 membered heteroalkylene. In embodiments, L^{1D} is independently substituted or unsubstituted 2 to 8 membered heteroalkylene. In embodiments, L^{1D} is independently substituted 2 to 8 membered heteroalkylene. In embodiments, L^{1D} is independently unsubstituted 2 to 8 membered heteroalkylene. In embodiments, L^{1D} is independently substituted or unsubstituted 2 to 6 membered heteroalkylene. In embodiments, L^{1D} is independently substituted 2 to 6 membered heteroalkylene. In embodiments, L^{1D} is independently unsubstituted 2 to 6 membered heteroalkylene. In embodiments, L^{1D} is independently substituted or unsubstituted 4 to 6 membered heteroalkylene. In embodiments, L^{1D} is independently substituted 4 to 6 membered heteroalkylene. In embodiments, L^{1D} is independently unsubstituted 4 to 6 membered heteroalkylene. In embodiments, L^{1D} is independently substituted or unsubstituted 2 to 3 membered heteroalkylene. In embodiments, L^{1D} is independently substituted 2 to 3 membered heteroalkylene. In embodiments, L^{1D} is independently unsubstituted 2 to 3 membered heteroalkylene. In embodiments, L^{1D} is independently substituted or unsubstituted 4 to 5 membered heteroalkylene. In embodiments, L^{1D} is independently substituted 4 to 5 membered heteroalkylene. In embodiments, L^{1D} is independently unsubstituted 4 to 5 membered heteroalkylene.

In embodiments, L^{1E} is a bond, —N(R²⁰)—, —O—, —S—, —C(O)—, —N(R²⁰)C(O)—, —C(O)N(R²¹)—, —N(R²⁰)C(O)N(R²¹)—, —C(O)O—, —OC(O)—, —N(R²⁰)C(O)O—, —OC(O)N(R²¹)—, —OPO₂—O—, —O—P(O)(S)—O—, —O—P(O)(R²²)—O—, —O—P(S)(R²²)—O—, —O—P(O)(NR²⁰R²¹)—N—, —O—P(S)(NR²⁰R²¹)—N—, —O—P(O)(NR²⁰R²¹)—O—, —O—P(S)(NR²⁰R²¹)—O—, —P(O)(NR²⁰R²¹)—N—, —P(S)(NR²⁰R²¹)—N—, —P(O)(NR²⁰R²¹)—O—, —P(S)(NR²⁰R²¹)—O—, —S—S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, L^{1E} is a bond. In embodiments, L^{1E} is —N(R²⁰)—. In embodiments, L^{1E} is —O— or —S—. In embodiments, L^{1E} is —C(O)—. In embodiments, L^{1E} is —N(R²⁰)C(O)— or —C(O)N(R²¹)—. In embodiments, L^{1E} is —N(R²⁰)C(O)N(R²¹)—. In embodiments, L^{1E} is —C(O)O— or —OC(O)—. In embodiments, L^{1E} is —N(R²⁰)C(O)O— or —OC(O)N(R²¹)—. In embodiments, L^{1E} is —PO₂—O—, —O—P(O)(S)—O—, —O—P(O)(R²²)—O—, —O—P(O)(NR²⁰R²¹)—N—, or O—P(O)(NR²⁰R²¹)—O—. In embodiments, L^{1E} is —P(O)(NR²⁰R²¹)—N—, —P(S)(NR²⁰R²¹)—N—, —P(O)(NR²⁰R²¹)—O— or —P(S)(NR²⁰R²¹)—O—. In embodiments, L^{1E} is —S—S—.

In embodiments, L^{1E} is independently substituted or unsubstituted alkylene (e.g., C₁-C₂₀, C₁-C₁₂, C₁-C₈, C₁-C₆, C₁-C₄, or C₁-C₂). In embodiments, L^{1E} is independently substituted alkylene (e.g., C₁-C₂₀, C₁-C₁₂, C₁-C₈, C₁-C₆, C₁-C₄, or C₁-C₂). In embodiments, L^{1E} is independently unsubstituted alkylene (e.g., C₁-C₂₀, C₁-C₁₂, C₁-C₈, C₁-C₆, C₁-C₄, or C₁-C₂). In embodiments, L^{1E} is independently substituted or unsubstituted C₁-C₂₀ alkylene. In embodiments, L^{1E} is independently substituted C₁-C₂₀ alkylene. In embodiments, L^{1E} is independently unsubstituted C₁-C₂₀ alkylene. In embodiments, L^{1E} is independently substituted or unsubstituted C₁-C₁₂ alkylene. In embodiments, L^{1E} is independently substituted C₁-C₁₂ alkylene. In embodiments, L^{1E} is independently unsubstituted C₁-C₁₂ alkylene. In embodiments, L^{1E} is independently substituted or unsubstituted C₁-C₈ alkylene. In embodiments, L^{1E} is independently substituted C₁-C₈ alkylene. In embodiments, L^{1E} is independently unsubstituted C₁-C₈ alkylene. In embodiments, L^{1E} is independently substituted or unsubstituted C₁-C₆ alkylene. In embodiments, L^{1E} is independently substituted C₁-C₆ alkylene. In embodiments, L^{1E} is independently unsubstituted C₁-C₆ alkylene. In embodiments, L^{1E} is independently substituted or unsubstituted C₁-C₄ alkylene. In embodiments, L^{1E} is independently substituted C₁-C₄ alkylene. In embodiments, L^{1E} is independently unsubstituted C₁-C₄ alkylene. In embodiments, L^{1E} is independently substituted or unsubstituted ethylene. In embodiments, L^{1E} is independently substituted ethylene. In embodiments, L^{1E} is independently unsubstituted ethylene. In embodiments, L^{1E} is independently substituted or unsubstituted methylene. In embodiments, L^{1E} is independently substituted methylene. In embodiments, L^{1E} is independently unsubstituted methylene.

In embodiments, L^{1E} is independently substituted or unsubstituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L^{1E} is independently substituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L^{1E} is independently unsubstituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L^{1E} is independently substituted or unsubstituted 2 to 20 membered heteroalkylene. In embodiments, L^{1E} is independently substituted 2 to 20 membered heteroalkylene. In embodiments, L^{1E} is independently unsubstituted 2 to 20 membered heteroalkylene. In embodiments, L^{1E} is independently substituted or unsubstituted 2 to 8 membered heteroalkylene. In embodiments, L^{1E} is independently substituted 2 to 8 membered heteroalkylene. In embodiments, L^{1E} is independently unsubstituted 2 to 8 membered heteroalkylene. In embodiments, L^{1E} is independently substituted or unsubstituted 2 to 6 membered heteroalkylene. In embodiments, L^{1E} is independently substituted 2 to 6 membered heteroalkylene. In embodiments, L^{1E} is independently unsubstituted 2 to 6 membered heteroalkylene. In embodiments, L^{1E} is independently substituted or unsubstituted 4 to 6 membered heteroalkylene. In embodiments, L^{1E} is independently substituted 4 to 6 membered heteroalkylene. In embodiments, L^{1E} is independently unsubstituted 4 to 6 membered heteroalkylene. In embodiments, L^{1E} is independently substituted or unsubstituted 2 to 3 membered heteroalkylene. In embodiments, L^{1E} is independently substituted 2 to 3 membered heteroalkylene. In embodiments, L^{1E} is independently unsubstituted 2 to 3 membered heteroalkylene. In embodiments, L^{1E} is independently substituted or unsubstituted 4 to 5 membered heteroalkylene. In embodiments, L^{1E} is independently substituted 4 to 5 membered heteroalkylene. In embodiments, L^{1E} is independently unsubstituted 4 to 5 membered heteroalkylene.

In embodiments, each R²⁰, R²¹ and R²² is independently hydrogen or unsubstituted C₁-C₁₀ alkyl.

In embodiments, R²⁰ is independently hydrogen or unsubstituted C₁-C₁₀ alkyl. In embodiments, R²⁰ is independently hydrogen. In embodiments, R²⁰ is unsubstituted C₁-C₁₀ alkyl. In embodiments, R²⁰ is unsubstituted C₁-C₈ alkyl. In embodiments, R²⁰ is unsubstituted C₁-C₆ alkyl. In embodiments, R²⁰ is unsubstituted C₁-C₅ alkyl. In embodiments, R²⁰ is unsubstituted C₁-C₄ alkyl. In embodiments, R²⁰ is unsubstituted C₁-C₃ alkyl. In embodiments, R²⁰ is unsubstituted methyl. In embodiments, R²⁰ is unsubstituted ethyl. In embodiments, R²⁰ is unsubstituted propyl. In embodiments, R²⁰ is unsubstituted isopropyl. In embodiments, R²⁰ is unsubstituted n-butyl. In embodiments, R²⁰ is unsubstituted t-butyl. In embodiments, R²⁰ is unsubstituted 2-butyl. In embodiments, R²⁰ is unsubstituted isobutyl.

In embodiments, R²¹ is independently hydrogen or unsubstituted C₁-C₁₀ alkyl. In embodiments, R²¹ is independently hydrogen. In embodiments, R²¹ is unsubstituted C₁-C₁₀ alkyl. In embodiments, R²¹ is unsubstituted C₁-C₈ alkyl. In embodiments, R²¹ is unsubstituted C₁-C₆ alkyl. In embodiments, R²¹ is unsubstituted C₁-C₅ alkyl. In embodiments, R²¹ is unsubstituted C₁-C₄ alkyl. In embodiments, R²¹ is unsubstituted C₁-C₃ alkyl. In embodiments, R²¹ is unsubstituted methyl. In embodiments, R²¹ is unsubstituted ethyl. In embodiments, R²¹ is unsubstituted propyl. In embodiments, R²¹ is unsubstituted isopropyl. In embodiments, R²¹ is unsubstituted n-butyl. In embodiments, R²¹ is unsubstituted t-butyl. In embodiments, R²¹ is unsubstituted 2-butyl. In embodiments, R²¹ is unsubstituted isobutyl.

In embodiments, R²² is independently hydrogen or unsubstituted C₁-C₁₀ alkyl. In embodiments, R²² is independently hydrogen. In embodiments, R²² is unsubstituted C₁-C₁₀ alkyl. In embodiments, R²² is unsubstituted C₁-C₈ alkyl. In embodiments, R²² is unsubstituted C₁-C₆ alkyl. In embodiments, R²² is unsubstituted C₁-C₅ alkyl. In embodiments, R²² is unsubstituted C₁-C₄ alkyl. In embodiments, R²² is unsubstituted C₁-C₃ alkyl. In embodiments, R²² is unsubstituted methyl. In embodiments, R²² is unsubstituted ethyl. In embodiments, R²² is unsubstituted propyl. In embodiments, R²² is unsubstituted isopropyl. In embodiments, R²² is unsubstituted n-butyl. In embodiments, R²² is unsubstituted t-butyl. In embodiments, R²² is unsubstituted 2-butyl. In embodiments, R²² is unsubstituted isobutyl.

In embodiments, each of R²⁰, R²¹ and R²² is independently hydrogen or unsubstituted C₁-C₃ alkyl. In embodiments, R²⁰ is hydrogen and each R²¹ and R²² is independently unsubstituted C₁-C₃ alkyl. In embodiments, R²¹ is hydrogen and each R²⁰ and R²² is independently unsubstituted C₁-C₃ alkyl. In embodiments, R²² is hydrogen and each R²⁰ and R²¹ is independently unsubstituted C₁-C₃ alkyl. In embodiments, R²⁰, R²¹ and R²² are hydrogen. In embodiments, R²⁰ is unsubstituted C₁-C₃ alkyl and R²¹ and R²² hydrogen. In embodiments, R²¹ is unsubstituted C₁-C₃ alkyl and R²⁰ and R²² hydrogen. In embodiments, R²² is unsubstituted C₁-C₃ alkyl and R²⁰ and R²¹ hydrogen. In embodiments, each of R²⁰, R²¹ and R²² are independently unsubstituted C₁-C₃ alkyl.

In embodiments, L² is independently an unsubstituted C₂-C₂₄ alkylene. In embodiments, L² is independently an unsubstituted C₂-C₂₂ alkylene. In embodiments, L² is independently an unsubstituted C₅-C₂₂ alkylene. In embodiments, L² is independently an unsubstituted C₁₀-C₂₂ alkylene. In embodiments, L² is independently an unsubstituted C₁₂-C₂₂ alkylene. In embodiments, L² is independently an unsubstituted C₁₀-C₂₀ alkylene. In embodiments, L² is independently an unsubstituted C₁₂-C₂₀ alkylene. In embodiments, L² is independently an unsubstituted C₁₀-C₁₈ alkylene. In embodiments, L² is independently an unsubstituted C₁₂-C₁₈ alkylene. In embodiments, L² is independently an unsubstituted C₁₀-C₁₆ alkylene. In embodiments, L² is independently an unsubstituted C₁₂-C₁₆ alkylene. In embodiments, L² is independently an unsubstituted C₁₄-C₁₆ alkylene. In embodiments, L² is independently an unsubstituted C₁₄-C₁₅ alkylene. In embodiments, L² is independently an unsubstituted C₁₄ alkylene. In embodiments, L² is independently an unsubstituted C₁₅ alkylene. In embodiments, L² is independently an unsubstituted C₁₆ alkylene.

In embodiments, L² is independently an unsubstituted unbranched C₂-C₂₄ alkylene. In embodiments, L² is independently an unsubstituted unbranched C₂-C₂₂ alkylene. In embodiments, L² is independently an unsubstituted unbranched C₅-C₂₂ alkylene. In embodiments, L² is independently an unsubstituted unbranched C₁₀-C₂₂ alkylene. In embodiments, L² is independently an unsubstituted unbranched C₁₂-C₂₂ alkylene. In embodiments, L² is independently an unsubstituted unbranched C₁₀-C₂₀ alkylene. In embodiments, L² is independently an unsubstituted unbranched C₁₂-C₂₀ alkylene. In embodiments, L² is independently an unsubstituted unbranched C₁₀-C₁₈ alkylene. In embodiments, L² is independently an unsubstituted unbranched C₁₂-C₁₈ alkylene. In embodiments, L² is independently an unsubstituted unbranched C₁₀-C₁₆ alkylene. In embodiments, L² is independently an unsubstituted unbranched C₁₂-C₁₆ alkylene. In embodiments, L² is independently an unsubstituted unbranched C₁₄-C₁₆ alkylene. In embodiments, L² is independently an unsubstituted unbranched C₁₄-C₁₅ alkylene. In embodiments, L² is independently an unsubstituted unbranched C₁₄ alkylene. In embodiments, L² is independently an unsubstituted unbranched C₁₅ alkylene. In embodiments, L² is independently an unsubstituted unbranched C₁₆ alkylene.

In embodiments, L² is independently an unsubstituted ununbranched saturated C₂-C₂₄ alkylene. In embodiments, L² is independently an unsubstituted ununbranched saturated C₂-C₂₂ alkylene. In embodiments, L² is independently an unsubstituted ununbranched saturated C₅-C₂₂ alkylene. In embodiments, L² is independently an unsubstituted ununbranched saturated C₁₀-C₂₂ alkylene. In embodiments, L² is independently an unsubstituted ununbranched saturated C₁₂-C₂₂ alkylene. In embodiments, L² is independently an unsubstituted ununbranched saturated C₁₀-C₂₀ alkylene. In embodiments, L² is independently an unsubstituted ununbranched saturated C₁₂-C₂₀ alkylene. In embodiments, L² is independently an unsubstituted ununbranched saturated C₁₀-C₁₈ alkylene. In embodiments, L² is independently an unsubstituted ununbranched saturated C₁₂-C₁₈ alkylene. In embodiments, L² is independently an unsubstituted ununbranched saturated C₁₀-C₁₆ alkylene. In embodiments, L² is independently an unsubstituted ununbranched saturated C₁₂-C₁₆ alkylene. In embodiments, L² is independently an unsubstituted ununbranched saturated C₁₄-C₁₆ alkylene. In embodiments, L² is independently an unsubstituted ununbranched saturated C₁₄-C₁₅ alkylene. In embodiments, L² is independently an unsubstituted ununbranched saturated C₁₄ alkylene. In embodiments, L² is independently an unsubstituted ununbranched saturated C₁₅ alkylene. In embodiments, L² is independently an unsubstituted ununbranched saturated C₁₆ alkylene.

In embodiments, L² is independently an unsubstituted ununbranched unsaturated C₂-C₂₄ alkylene. In embodiments, L² is independently an unsubstituted ununbranched unsaturated C₂-C₂₂ alkylene. In embodiments, L² is independently an unsubstituted ununbranched unsaturated C₅-C₂₂ alkylene. In embodiments, L² is independently an unsubstituted ununbranched unsaturated C₁₀-C₂₂ alkylene. In embodiments, L² is independently an unsubstituted ununbranched unsaturated C₁₂-C₂₂ alkylene. In embodiments, L² is independently an unsubstituted ununbranched unsaturated C₁₀-C₂₀ alkylene. In embodiments, L² is independently an unsubstituted ununbranched unsaturated C₁₂-C₂₀ alkylene. In embodiments, L² is independently an unsubstituted ununbranched unsaturated C₁₀-C₁₈ alkylene. In embodiments, L² is independently an unsubstituted ununbranched unsaturated C₁₂-C₁₈ alkylene. In embodiments, L² is independently an unsubstituted ununbranched unsaturated C₁₀-C₁₆ alkylene. In embodiments, L² is independently an unsubstituted ununbranched unsaturated C₁₂-C₁₆ alkylene. In embodiments, L² is independently an unsubstituted ununbranched unsaturated C₁₄-C₁₆ alkylene. In embodiments, L² is independently an unsubstituted ununbranched unsaturated C₁₄-C₁₅ alkylene. In embodiments, L² is independently an unsubstituted ununbranched unsaturated C₁₄ alkylene. In embodiments, L² is independently an unsubstituted ununbranched unsaturated C₁₅ alkylene. In embodiments, L² is independently an unsubstituted ununbranched unsaturated C₁₆ alkylene.

In embodiments, the largest dimension of L¹ is less than 200 angstroms. In embodiments, the largest dimension of L¹ is less than 190 angstroms. In embodiments, the largest dimension of L¹ is less than 180 angstroms. In embodiments, the largest dimension of L¹ is less than 170 angstroms. In embodiments, the largest dimension of L¹ is less than 160 angstroms. In embodiments, the largest dimension of L¹ is less than 150 angstroms. In embodiments, the largest dimension of L¹ is less than 140 angstroms. In embodiments, the largest dimension of L¹ is less than 130 angstroms. In embodiments, the largest dimension of L¹ is less than 120 angstroms. In embodiments, the largest dimension of L¹ is less than 110 angstroms. In embodiments, the largest dimension of L¹ is less than 100 angstroms. In embodiments, the largest dimension of L¹ is less than 90 angstroms. In embodiments, the largest dimension of L¹ is less than 80 angstroms. In embodiments, the largest dimension of L¹ is less than 70 angstroms. In embodiments, the largest dimension of L¹ is less than 60 angstroms. In embodiments, the largest dimension of L¹ is less than 50 angstroms. In embodiments, the largest dimension of L¹ is less than 40 angstroms. In embodiments, the largest dimension of L¹ is less than 30 angstroms. In embodiments, the largest dimension of L¹ is less than 20 angstroms. In embodiments, the largest dimension of L¹ is less than 10 angstroms.

In embodiments, the largest dimension of each of L^{1A}, L^{1B}, L^{1C}, L^{1D}, and L^{1E} is independently less than 50 angstroms. In embodiments, the largest dimension of each of L^{1A}, L^{1B}, L^{1C}, L^{1D}, and L^{1E} is independently less than 40 angstroms. In embodiments, the largest dimension of each of L^{1A}, L^{1B}, L^{1C}, L^{1D}, and L^{1E} is independently less than 30 angstroms. In embodiments, the largest dimension of each of L^{1A}, L^{1B}, L^{1C}, L^{1D}, and L^{1E} is independently less than 20 angstroms. In embodiments, the largest dimension of each of L^{1A}, L^{1B}, L^{1C}, L^{1D}, and L^{1E} is independently less than 10 angstroms.

In embodiments, the largest dimension of L^{1A} is independently less than 50 angstroms. In embodiments, the largest dimension of L^{1A} is independently less than 40 angstroms. In embodiments, the largest dimension of L^{1A} is independently less than 30 angstroms. In embodiments, the largest dimension of L^{1A} is independently less than 20 angstroms. In embodiments, the largest dimension of L^{1A} is independently less than 10 angstroms.

In embodiments, the largest dimension of L^{1B} is independently less than 50 angstroms. In embodiments, the largest dimension of L^{1B} is independently less than 40 angstroms. In embodiments, the largest dimension of L^{1B} is independently less than 30 angstroms. In embodiments, the largest dimension of L^{1B} is independently less than 20 angstroms. In embodiments, the largest dimension of L^{1B} is independently less than 10 angstroms.

In embodiments, the largest dimension of L^{1C} is independently less than 50 angstroms. In embodiments, the largest dimension of L^{1C} is independently less than 40 angstroms. In embodiments, the largest dimension of L^{1C} is independently less than 30 angstroms. In embodiments, the largest dimension of L^{1C} is independently less than 20 angstroms. In embodiments, the largest dimension of L^{1C} is independently less than 10 angstroms.

In embodiments, the largest dimension of L^{1D} is independently less than 50 angstroms. In embodiments, the largest dimension of L^{1D} is independently less than 40 angstroms. In embodiments, the largest dimension of L^{1D} is independently less than 30 angstroms. In embodiments, the largest dimension of L^{1D} is independently less than 20 angstroms. In embodiments, the largest dimension of L^{1D} is independently less than 10 angstroms.

In embodiments, the largest dimension of L^{1E} is independently less than 50 angstroms. In embodiments, the largest dimension of L^{1E} is independently less than 40 angstroms. In embodiments, the largest dimension of L^{1E} is independently less than 30 angstroms. In embodiments, the largest dimension of L^{1E} is independently less than 20 angstroms. In embodiments, the largest dimension of L^{1E} is independently less than 10 angstroms.

In embodiments, the nucleic acid (A) is an oligonucleotide. In embodiments, one L^{1A} is attached to a 3′ carbon of the oligonucleotide. In embodiments, one L^{1A} is attached to a 3′ nitrogen of the oligonucleotide (e.g., the 3′ nitrogen of a morpholino moiety). In embodiments, one L^{1A} is attached to a 5′ carbon of the oligonucleotide. In embodiments, one L^{1A} is attached to a 6′ carbon of the oligonucleotide (e.g., the 6′ carbon of a morpholino moiety). In embodiments, one L^{1A} is attached to a 2′ carbon of the oligonucleotide. In embodiments, one L^{1A} is attached to a nucleobase of the oligonucleotide.

In embodiments, at least one L^{1A} is attached to a 3′ carbon of the oligonucleotide at a 3′ end. In embodiments, at least one L^{1A} is attached to a 3′ nitrogen of the oligonucleotide (e.g., the 3′ nitrogen of a morpholino moiety) at its 3′ end. In embodiments, at least one L^{1A} is attached to a 5′ carbon of the oligonucleotide at its 5′ end. In embodiments, at least one L^{1A} is attached to a 6′ carbon of the oligonucleotide (e.g., the 6′ carbon of a morpholino moiety) at its 5′ end.

In embodiments, the nucleic acid (A) is a double-stranded oligonucleotide. In embodiments, one L^{1A} is attached to a 3′ carbon of the double-stranded oligonucleotide. In embodiments, one L^{1A} is attached to a 3′ carbon of the double-stranded oligonucleotide at either of its 3′ ends. In embodiments, one L^{1A} is attached to a 3′ carbon of the double-stranded oligonucleotide at the 3′end of its antisense strand. In embodiments, one L^{1A} is attached to a 3′ carbon of the double-stranded oligonucleotide at the 3′end of its sense strand.

In embodiments, one L^{1A} is attached to a 3′ nitrogen of the double-stranded oligonucleotide (e.g., the 3′ nitrogen of a morpholino moiety) at either of its 3′ ends. In embodiments, one L^{1A} is attached to a 3′ nitrogen of the double-stranded oligonucleotide (e.g., the 3′ nitrogen of a morpholino moiety) at the 3′end of its antisense strand. In embodiments, one L^{1A} is attached to a 3′ nitrogen of the double-stranded oligonucleotide (e.g., the 3′ nitrogen of a morpholino moiety) at the 3′end of its sense strand.

In embodiments, one L^{1A} is attached to a 5′ carbon of the double-stranded oligonucleotide at either of its 5′ ends. In embodiments, one L^{1A} is attached to a 5′ carbon of the double-stranded oligonucleotide at the 5′ end of its antisense strand. In embodiments, one L^{1A} is attached to a 5′ carbon of the double-stranded oligonucleotide at the 5′ end of its sense strand.

In embodiments, one L^{1A} is attached to a 6′ carbon of the double-stranded oligonucleotide (e.g., the 6′ carbon of a morpholino moiety) at either of its 5′ ends. In embodiments, one L^{1A} is attached to a 6′ carbon of the double-stranded oligonucleotide (e.g., the 6′ carbon of a morpholino moiety) at the 5′ end of its antisense strand. In embodiments, one L^{1A} is attached to a 6′ carbon of the double-stranded oligonucleotide (e.g., the 6′ carbon of a morpholino moiety) at the 5′ end of its sense strand.

In embodiments, one L^{1A} is attached to a 2′ carbon of the double-stranded oligonucleotide. In embodiments, one L^{1A} is attached to a 2′ carbon of the double-stranded oligonucleotide at either of its 5′ ends. In embodiments, one L^{1A} is attached to a 2′ carbon at the 5′ end of the sense strand. In embodiments, one L^{1A} is attached to a 2′ carbon at the 5′ end of the antisense strand. In embodiments, one L^{1A} is attached to a 2′ carbon of the double-stranded oligonucleotide at either of its 3′ ends. In embodiments, one L^{1A} is attached to a 2′ carbon at the 3′ end of the sense strand. In embodiments, one L^{1A} is attached to a 2′ carbon at the 3′ end of the antisense strand.

In embodiments, one L^{1A} is attached to a nucleobase of the double-stranded oligonucleotide. In embodiments, one L^{1A} is attached to a nucleobase of the sense strand of the double-stranded oligonucleotide. In embodiments, one L^{1A} is attached to a nucleobase of the antisense strand of the double-stranded oligonucleotide. In embodiments, one L^{1A} is attached to a nucleobase of the double-stranded oligonucleotide at either of its 3′ ends. In embodiments, one L^{1A} is attached to a nucleobase of the double-stranded oligonucleotide at the 3′end of its antisense strand. In embodiments, one L^{1A} is attached to a nucleobase of the double-stranded oligonucleotide at the 3′end of its sense strand. In embodiments, one L^{1A} is attached to a nucleobase of the double-stranded oligonucleotide at either of its 5′ ends. In embodiments, one L^{1A} is attached to a nucleobase of the double-stranded oligonucleotide at the 5′ end of its antisense strand. In embodiments, one L^{1A} is attached to a nucleobase of the double-stranded oligonucleotide at the 5′ end of its sense strand.

In embodiments, the nucleic acid (A) is a single-stranded oligonucleotide. In embodiments, one L^{1A} is attached to a 3′ carbon of the single-stranded oligonucleotide at the 3′ end.

In embodiments, one L^{1A} is attached to a 3′ nitrogen of the single-stranded oligonucleotide (e.g., the 3′ nitrogen of a morpholino moiety) at the 3′ end of the single-stranded oligonucleotide.

In embodiments, one L^{1A} is attached to a 5′ carbon of the single-stranded oligonucleotide at the 5′ end.

In embodiments, one L^{1A} is attached to a 6′ carbon of the single-stranded oligonucleotide (e.g., the 6′ carbon of a morpholino moiety) at the 5′ end.

In embodiments, one L^{1A} is attached to a 2′ carbon of the single-stranded oligonucleotide. In embodiments, on L^{1A} is attached to a 2′ carbon of the single-stranded oligonucleotide at its 5′ end. In embodiments, one L^{1A} is attached to a 2′ carbon of the single-stranded oligonucleotide at its 3′ end.

In embodiments, one L^{1A} is attached to a nucleobase of the single-stranded oligonucleotide. In embodiments, one L^{1A} is attached to a nucleobase of the single-stranded oligonucleotide at the of 3′ end. In embodiments, one L^{1A} is attached to a nucleobase of the single-stranded oligonucleotide at the 5′ end.

In embodiments, L^{1A} is independently —O—, —C(O)—, —C(O)O—, —OC(O)—, —OPO₂—O—, —O—P(O)(S)—O—, —O—P(O)(CH₃)—O—, —O—P(O)(N(CH₃)₂)—N—, —O—P(O)(N(CH₃)₂)—O—, —O—P(S)(N(CH₃)₂)—N—, —O—P(S)(N(CH₃)₂)—O—, —P(O)(N(CH₃)₂)—N—, —P(O)(N(CH₃)₂)—O—, —P(S)(N(CH₃)₂)—N—, —P(S)(N(CH₃)₂)—O—, substituted or unsubstituted alkylene, or substituted or unsubstituted heteroalkylene. In embodiments, L^{1A} is independently —O—, —C(O)—, —C(O)O— or —OC(O)—. In embodiments, L^{1A} is independently —OPO₂—O—, —O—P(O)(S)—O—, —O—P(O)(CH₃)—O—, or —O—P(S)(CH₃)—O—. In embodiments, L^{1A} is independently —O—P(O)(N(CH₃)₂)—N—, —O—P(O)(N(CH₃)₂)—O—, —O—P(S)(N(CH₃)₂)—N—, or —O—P(S)(N(CH₃)₂)—O—. In embodiments, L^{1A} is independently —P(O)(N(CH₃)₂)—N—, —P(O)(N(CH₃)₂)—O—, —P(S)(N(CH₃)₂)—N—, or —P(S)(N(CH₃)₂)—O—.

In embodiments, L^{1A} is independently substituted or unsubstituted C₁-C₂₀ alkylene. In embodiments, L^{1A} is independently substituted or unsubstituted C₁-C₁₂ alkylene. In embodiments, L^{1A} is independently substituted or unsubstituted C₁-C₈ alkylene. In embodiments, L^{1A} is independently substituted or unsubstituted C₁-C₆ alkylene. In embodiments, L^{1A} is independently substituted or unsubstituted C₁-C₄ alkylene. In embodiments, L^{1A} is independently substituted or unsubstituted C₁-C₂ alkylene.

In embodiments, L^{1A} is independently substituted or unsubstituted 2 to 20 membered heteroalkylene. In embodiments, L^{1A} is independently substituted or unsubstituted 2 to 16 membered heteroalkylene. In embodiments, L^{1A} is independently substituted or unsubstituted 2 to 12 membered heteroalkylene. In embodiments, L^{1A} is independently substituted or unsubstituted 2 to 10 membered heteroalkylene. In embodiments, L^{1A} is independently substituted or unsubstituted 2 to 8 membered heteroalkylene. In embodiments, L^{1A} is independently substituted or unsubstituted 2 to 6 membered heteroalkylene. In embodiments, L^{1A} is independently substituted or unsubstituted 2 to 4 membered heteroalkylene. In embodiments, L^{1A} is independently substituted or unsubstituted 2 to 3 membered heteroalkylene.

In embodiments, L^{1A} is independently

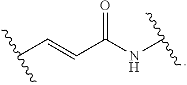 [SMILES: */C=C/C(=O)N*]

In embodiments, L^{1A} is independently —OPO₂—O—. In embodiments, L^{1A} is independently —O—P(O)(S)—O—. In embodiments, L^{1A} is independently —O—. In embodiments, L^{1A} is independently —S—.

). In embodiments, L^{1A} is attached to the 3′nitrogen of a morpholino moiety. In embodiments, L^{1A} is independently —C(O)—. In embodiments, L^{1A} is attached to the 6′ carbon of a morpholino moiety. In embodiments, L^{1A} is independently —O—P(O)(N(CH₃)₂)—N—. In embodiments, L^{1A} is independently —O—P(O)(N(CH₃)₂)—O—. In embodiments, L^{1A} is independently —P(O)(N(CH₃)₂)—N—. In embodiments, L^{1A} is independently —P(O)(N(CH₃)₂)—O—.

In embodiments, L^{1B} is independently substituted or unsubstituted alkylene or substituted or unsubstituted heteroalkylene. In embodiments, L^{1B} is independently substituted or unsubstituted C₁-C₂₀ alkylene. In embodiments, L^{1B} is independently substituted or unsubstituted C₁-C₁₂ alkylene. In embodiments, L^{1B} is independently substituted or unsubstituted C₁-C₈ alkylene. In embodiments, L^{1B} is independently substituted or unsubstituted C₁-C₆ alkylene. In embodiments, L^{1B} is independently substituted or unsubstituted C₁-C₄ alkylene. In embodiments, L^{1B} is independently substituted or unsubstituted C₁-C₂ alkylene.

In embodiments, L^{1B} is independently substituted or unsubstituted 2 to 20 membered heteroalkylene. In embodiments, L^{1B} is independently substituted or unsubstituted 2 to 16 membered heteroalkylene. In embodiments, L^{1B} is independently substituted or unsubstituted 2 to 12 membered heteroalkylene. In embodiments, L^{1B} is independently substituted or unsubstituted 2 to 10 membered heteroalkylene. In embodiments, L^{1B} is independently substituted or unsubstituted 2 to 8 membered heteroalkylene. In embodiments, L^{1B} is independently substituted or unsubstituted 2 to 6 membered heteroalkylene. In embodiments, L^{1B} is independently substituted or unsubstituted 2 to 4 membered heteroalkylene. In embodiments, L^{1B} is independently substituted or unsubstituted 2 to 3 membered heteroalkylene.

In embodiments, L^{1B} is independently -L¹⁰-NH—C(O)— or -L¹⁰-C(O)—NH—. L¹⁰ is substituted or unsubstituted alkylene.

In embodiments, L¹⁰ is independently substituted or unsubstituted alkylene (e.g., C₁-C₂₀, C₁-C₁₂, C₁-C₈, C₁-C₆, C₁-C₄, or C₁-C₂). In embodiments, L¹⁰ is independently substituted alkylene (e.g., C₁-C₂₀, C₁-C₁₂, C₁-C₈, C₁-C₆, C₁-C₄, or C₁-C₂). In embodiments, L¹⁰ is independently unsubstituted alkylene (e.g., C₁-C₂₀, C₁-C₁₂, C₁-C₈, C₁-C₆, C₁-C₄, or C₁-C₂). In embodiments, L¹⁰ is independently substituted or unsubstituted C₁-C₂₀ alkylene. In embodiments, L¹⁰ is independently substituted C₁-C₂₀ alkylene. In embodiments, L¹⁰ is independently hydroxy(OH)-substituted C₁-C₂₀ alkylene. In embodiments, L¹⁰ is independently hydroxymethyl-substituted C₁-C₂₀ alkylene. In embodiments, L¹⁰ is independently unsubstituted C₁-C₂₀ alkylene. In embodiments, L¹⁰ is independently substituted or unsubstituted C₁-C₁₂ alkylene. In embodiments, L¹⁰ is independently substituted C₁-C₁₂ alkylene. In embodiments, L¹⁰ is independently hydroxy(OH)-substituted C₁-C₁₂ alkylene. In embodiments, L¹⁰ is independently hydroxymethyl-substituted C₁-C₁₂ alkylene. In embodiments, L¹⁰ is independently unsubstituted C₁-C₁₂ alkylene. In embodiments, L¹⁰ is independently substituted or unsubstituted C₁-C₈ alkylene. In embodiments, L¹⁰ is independently substituted C₁-C₈ alkylene. In embodiments, L¹⁰ is independently hydroxy(OH)-substituted C₁-C₈ alkylene. In embodiments, L¹⁰ is independently hydroxymethyl-substituted C₁-C₈ alkylene. In embodiments, L¹⁰ is independently unsubstituted C₁-C₈ alkylene. In embodiments, L¹⁰ is independently substituted or unsubstituted C₁-C₆ alkylene. In embodiments, L¹⁰ is independently substituted C₁-C₆ alkylene. In embodiments, L¹⁰ is independently hydroxy(OH)-substituted C₁-C₆ alkylene. In embodiments, L¹⁰ is independently hydroxymethyl-substituted C₁-C₆ alkylene. In embodiments, L¹⁰ is independently unsubstituted C₁-C₆ alkylene. In embodiments, L¹⁰ is independently substituted or unsubstituted C₁-C₄ alkylene. In embodiments, L¹⁰ is independently substituted C₁-C₄ alkylene. In embodiments, L¹⁰ is independently hydroxy(OH)-substituted C₁-C₄ alkylene. In embodiments, L¹⁰ is independently hydroxymethyl-substituted C₁-C₄ alkylene. In embodiments, L¹⁰ is independently unsubstituted C₁-C₄ alkylene. In embodiments, L¹⁰ is independently substituted or unsubstituted C₁-C₂ alkylene. In embodiments, L¹⁰ is independently substituted C₁-C₂ alkylene. In embodiments, L¹⁰ is independently hydroxy(OH)-substituted C₁-C₂ alkylene. In embodiments, L¹⁰ is independently hydroxymethyl-substituted C₁-C₂ alkylene. In embodiments, L¹⁰ is independently unsubstituted C₁-C₂ alkylene.

In embodiments, L^{1B} is independently

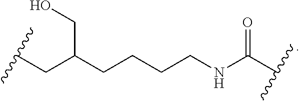 [SMILES: *CC(CO)CCCCNC(*)=O]

In embodiments, L^{1B} is independently

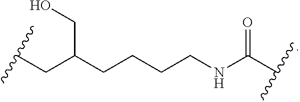 [SMILES: *CC(CO)CCCCNC(*)=O]

In embodiments, L^{1B} is independently

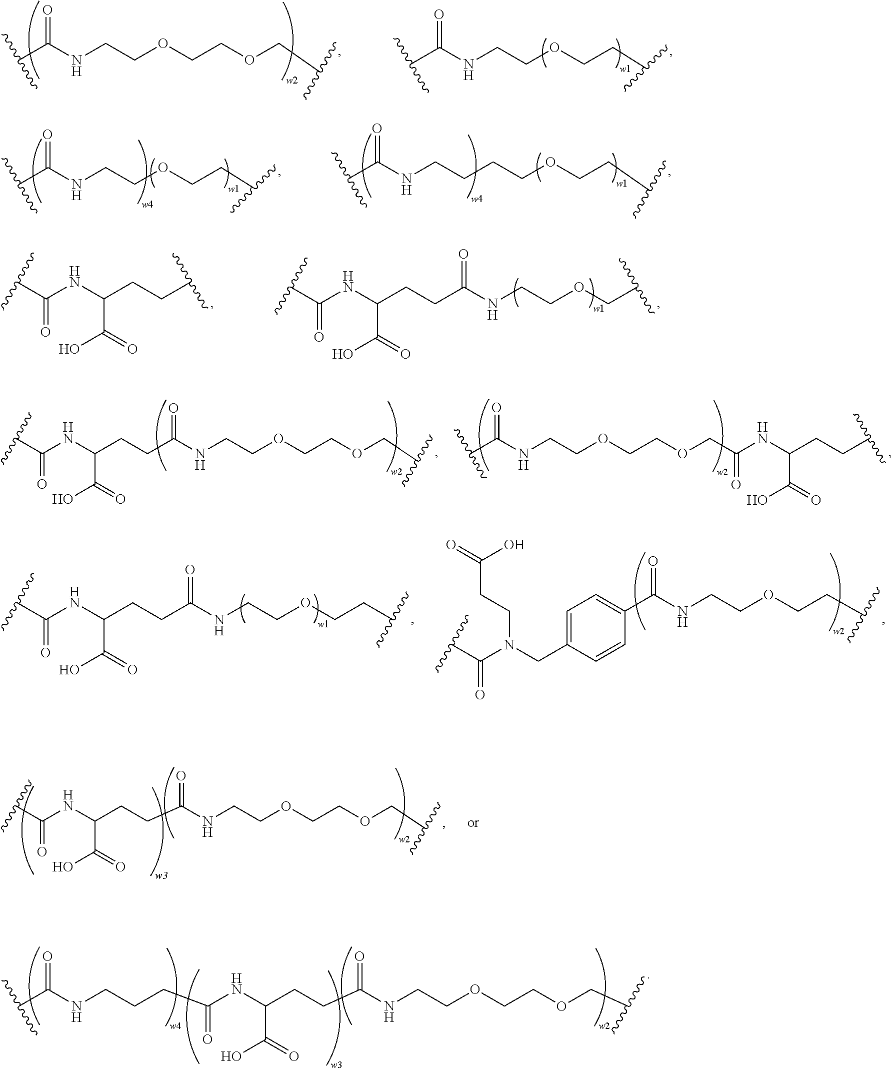

w1 is an integer from 0 to 10. w2 is an integer from 0 to 5. w3 is an integer from 0 to 5. w4 is an integer from 0 to 5.

In embodiments, L^{1B} is independently

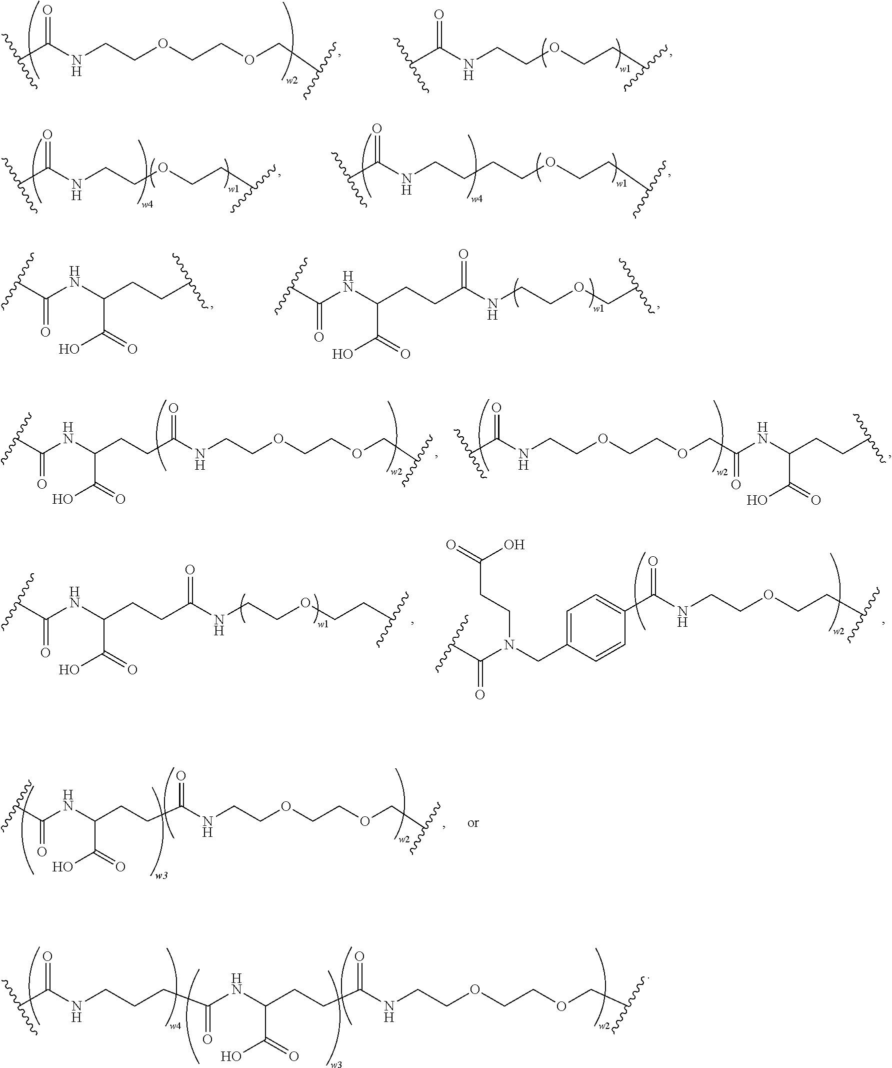

w1, w2, w3, and w4 are as described above.

In embodiments, w1 is 0. In embodiments, w1 is 1. In embodiments, w1 is 2. In embodiments, w1 is 3. In embodiments, w1 is 4. In embodiments, w1 is 5. In embodiments, w1 is 6. In embodiments, w1 is 7. In embodiments, w1 is 8. In embodiments, w1 is 9. In embodiments, w1 is 10. In embodiments, w2 is 0. In embodiments, w2 is 1. In embodiments, w2 is 2. In embodiments, w2 is 3. In embodiments, w2 is 4. In embodiments, w2 is 5. In embodiments, w3 is 0. In embodiments, w3 is 1. In embodiments, w3 is 2. In embodiments, w3 is 3. In embodiments, w3 is 4. In embodiments, w3 is 5. In embodiments, w4 is 0. In embodiments, w4 is 1. In embodiments, w4 is 2. In embodiments, w4 is 3. In embodiments, w4 is 4. In embodiments, w4 is 5.

In embodiments, L^{1B} is independently

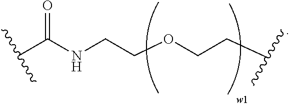

In embodiments, L^{1B} is independently

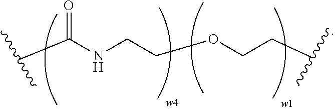

### In

embodiments, L^{1B} is independently

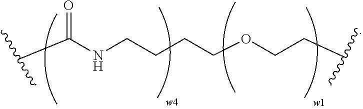

In embodiments, w4 is 0. In embodiments, w1 is 1. In embodiments, w1 is 2. In embodiments, w2 is 3. In embodiments, w4 is 0 and w1 is 2. In embodiments, w4 is 0 and w1 is 3. In embodiments, w4 is 0 and w1 is 4.

In embodiments, L^{1B} is independently

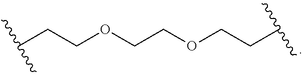 [SMILES: *CCOCCOCC*]

In embodiments, L^{1B} is independently

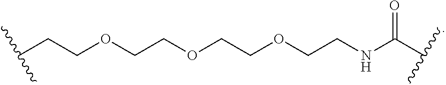 [SMILES: *CCOCCOCCOCCNC(*)=O]

In embodiments, -L^{1A}-L^{1B}- is independently —O-L¹⁰-NH—C(O)— or —O-L¹⁰-C(O)—NH—. L¹⁰ is independently substituted or unsubstituted alkylene, substituted or unsubstituted heteroalkylene, or substituted or unsubstituted heteroalkenylene. In embodiments, -L^{1A}-L^{1B}- is independently —O-L¹⁰-NH—C(O)—. In embodiments, -L^{1A}-L^{1B}- is independently —O-L¹⁰-C(O)—NH—.

In embodiments, L¹⁰ is independently substituted or unsubstituted alkylene (e.g., C₁-C₂₀, C₁-C₁₂, C₁-C₈, C₁-C₆, C₁-C₄, or C₁-C₂). In embodiments, L¹⁰ is independently substituted alkylene (e.g., C₁-C₂₀, C₁-C₁₂, C₁-C₈, C₁-C₆, C₁-C₄, or C₁-C₂). In embodiments, L¹⁰ is independently unsubstituted alkylene (e.g., C₁-C₂₀, C₁-C₁₂, C₁-C₈, C₁-C₆, C₁-C₄, or C₁-C₂). In embodiments, L¹⁰ is independently substituted or unsubstituted C₁-C₂₀ alkylene. In embodiments, L¹⁰ is independently substituted C₁-C₂ alkylene. In embodiments, L¹⁰ is independently hydroxy(OH)-substituted C₁-C₂ alkylene. In embodiments, L¹⁰ is independently hydroxymethyl-substituted C₁-C₂₀ alkylene. In embodiments, L¹⁰ is independently unsubstituted C₁-C₂₀ alkylene. In embodiments, L¹⁰ is independently substituted or unsubstituted C₁-C₁₂ alkylene. In embodiments, L¹⁰ is independently substituted C₁-C₁₂ alkylene. In embodiments, L¹⁰ is independently hydroxy(OH)-substituted C₁-C₁₂ alkylene. In embodiments, L¹⁰ is independently hydroxymethyl-substituted C₁-C₁₂ alkylene. In embodiments, L¹⁰ is independently unsubstituted C₁-C₁₂ alkylene. In embodiments, L¹⁰ is independently substituted or unsubstituted C₁-C₈ alkylene. In embodiments, L¹⁰ is independently substituted C₁-C₈ alkylene. In embodiments, L¹⁰ is independently hydroxy(OH)-substituted C₁-C₈ alkylene. In embodiments, L¹⁰ is independently hydroxymethyl-substituted C₁-C₈ alkylene. In embodiments, L¹⁰ is independently unsubstituted C₁-C₈ alkylene. In embodiments, L¹⁰ is independently substituted or unsubstituted C₅-C₈ alkylene. In embodiments, L¹⁰ is independently substituted C₅-C₈ alkylene. In embodiments, L¹⁰ is independently hydroxy(OH)-substituted C₅-C₈ alkylene. In embodiments, L¹⁰ is independently hydroxymethyl-substituted C₅-C₈ alkylene. In embodiments, L¹⁰ is independently unsubstituted C₅-C₈ alkylene. In embodiments, L¹⁰ is independently substituted or unsubstituted C₁-C₆ alkylene. In embodiments, L¹⁰ is independently substituted C₁-C₆ alkylene. In embodiments, L¹⁰ is independently hydroxy(OH)-substituted C₁-C₆ alkylene. In embodiments, L¹⁰ is independently hydroxymethyl-substituted C₁-C₆ alkylene. In embodiments, L¹⁰ is independently unsubstituted C₁-C₆ alkylene. In embodiments, L¹⁰ is independently substituted or unsubstituted C₁-C₄ alkylene. In embodiments, L¹⁰ is independently substituted C₁-C₄ alkylene. In embodiments, L¹⁰ is independently hydroxy(OH)-substituted C₁-C₄ alkylene. In embodiments, L¹⁰ is independently hydroxymethyl-substituted C₁-C₄ alkylene. In embodiments, L¹⁰ is independently unsubstituted C₁-C₄ alkylene. In embodiments, L¹⁰ is independently substituted or unsubstituted C₁-C₂ alkylene. In embodiments, L¹⁰ is independently substituted C₁-C₂ alkylene. In embodiments, L¹⁰ is independently hydroxy(OH)-substituted C₁-C₂alkylene. In embodiments, L¹⁰ is independently hydroxymethyl-substituted C₁-C₂ alkylene. In embodiments, L¹⁰ is independently unsubstituted C₁-C₂ alkylene.

In embodiments, -L^{1A}-L^{1B}- is independently

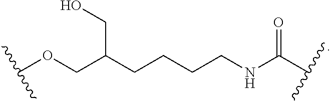 [SMILES: *OCC(CO)CCCCNC(*)=O]

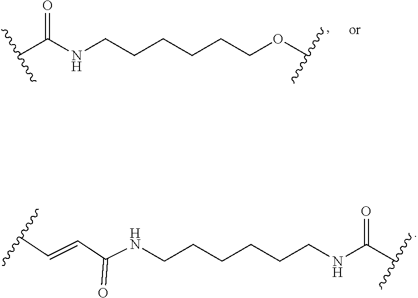 [SMILES: *OCCCCCCNC(*)=O ; */C=C/C(=O)NCCCCCCNC(*)=O]

In embodiments, -L^{1A}-L^{1B}- is independently

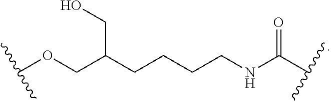 [SMILES: *OCC(CO)CCCCNC(*)=O]

In embodiments, -L^{1A}-L^{1B}- is independently

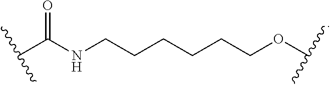 [SMILES: *OCCCCCCNC(*)=O]

In embodiments, -L^{1A}-L^{1B}- is independently

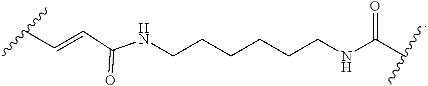 [SMILES: */C=C/C(=O)NCCCCCCNC(*)=O]

In embodiments, -L^{1A}-L^{1B}- is independently —PO₂—O-L¹⁰-NH—C(O)—, —OP(O)(S)—O-L¹⁰-NH—C(O)—, —OPO₂—O-L¹⁰-C(O)—NH— or —OP(O)(S)—O-L¹⁰-C(O)—NH—. In embodiments, L¹⁰ is independently substituted or unsubstituted alkylene. In embodiments, -L^{1A}-L^{1B}- is independently —PO₂—O-L¹⁰-NH—C(O)— or —OP(O)(S)—O-L¹⁰-NH—C(O)—. In embodiments, -L^{1A}-L^{1B}- is independently —PO₂—O-L¹⁰-C(O)—NH— or —OP(O)(S)—O-L¹⁰-C(O)—NH—. In embodiments, L¹⁰ is independently substituted or unsubstituted alkylene. In embodiments, L¹⁰ is independently substituted or unsubstituted C₅-C₈ alkylene. In embodiments, L¹⁰ is independently substituted C₅-C₈ alkylene. In embodiments, L¹⁰ is independently hydroxy (OH)-substituted C₅-C₈ alkylene. In embodiments, L¹⁰ is independently hydroxymethyl-substituted C₅-C₈ alkylene. In embodiments, L¹⁰ is independently unsubstituted C₅-C₈ alkylene.

In embodiments, -L^{1A}-L^{1B}- is independently

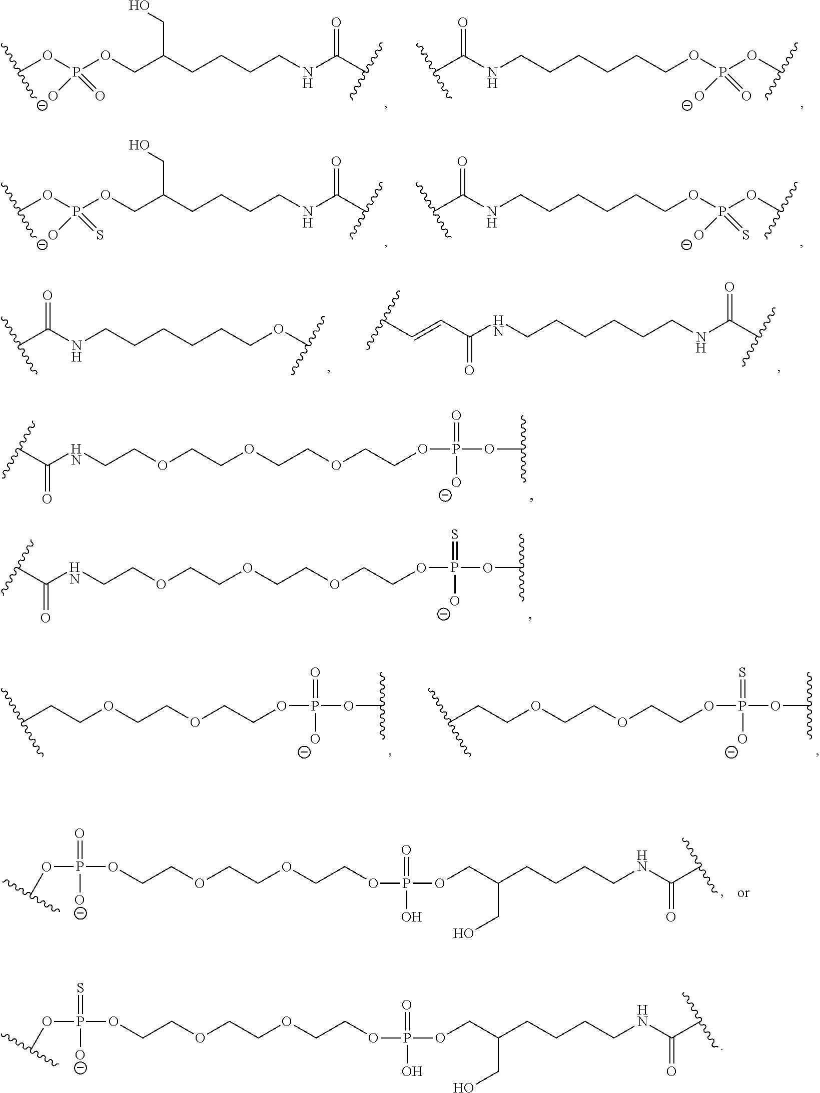

In embodiments, -L^{1A}-L^{1B}- is independently

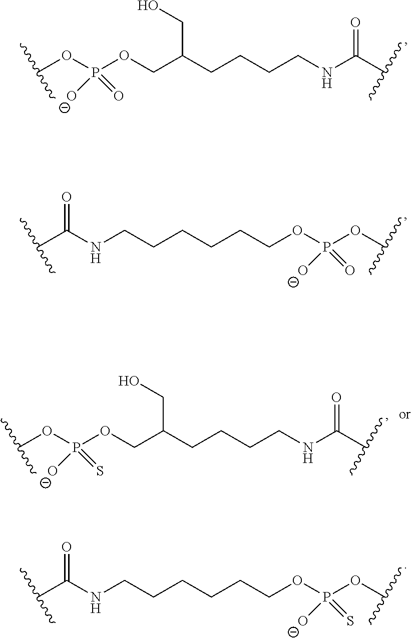 [SMILES: *OP(=O)([O-])OCC(CO)CCCCNC(*)=O ; *OP(=O)([O-])OCCCCCCNC(*)=O ; *OP([O-])(=S)OCC(CO)CCCCNC(*)=O ; *OP([O-])(=S)OCCCCCCNC(*)=O]

In embodiments, -L^{1A}-L^{1B}- is independently

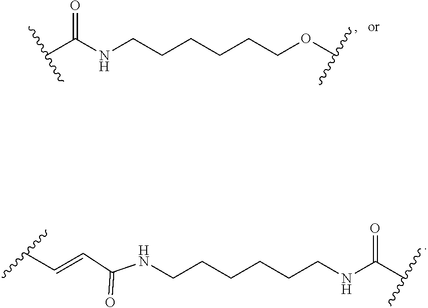 [SMILES: *OCCCCCCNC(*)=O ; */C=C/C(=O)NCCCCCCNC(*)=O]

In embodiments, -L^{1A}-L^{1B}- is independently

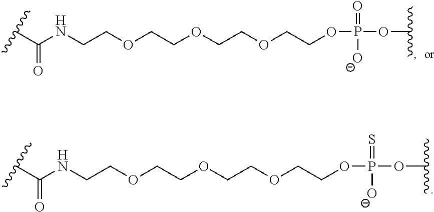 [SMILES: *OP(=O)([O-])OCCOCCOCCOCCNC(*)=O ; *OP([O-])(=S)OCCOCCOCCOCCNC(*)=O]

In embodiments, -L^{1A}-L^{1B} is independently

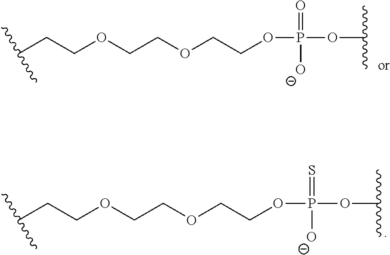 [SMILES: *CCOCCOCCOP(=O)([O-])O* ; *CCOCCOCCOP([O-])(=S)O*]

In embodiments, -L^{1A}-L^{1B}- is independently

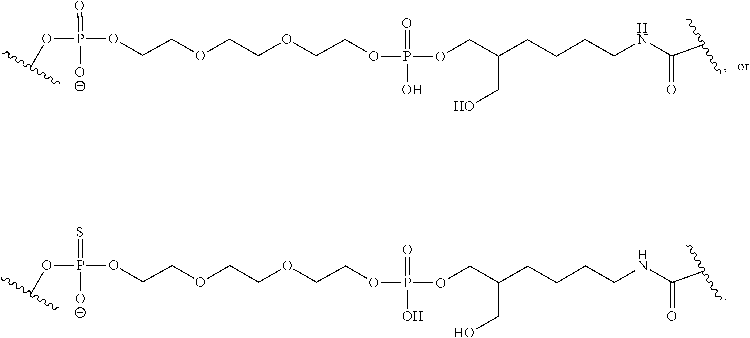 [SMILES: *OP(=O)([O-])OCCOCCOCCOP(=O)(O)OCC(CO)CCCCNC(*)=O ; *OP([O-])(=S)OCCOCCOCCOP(=O)(O)OCC(CO)CCCCNC(*)=O]

In embodiments, -L^{1A}-L^{1B}- is independently

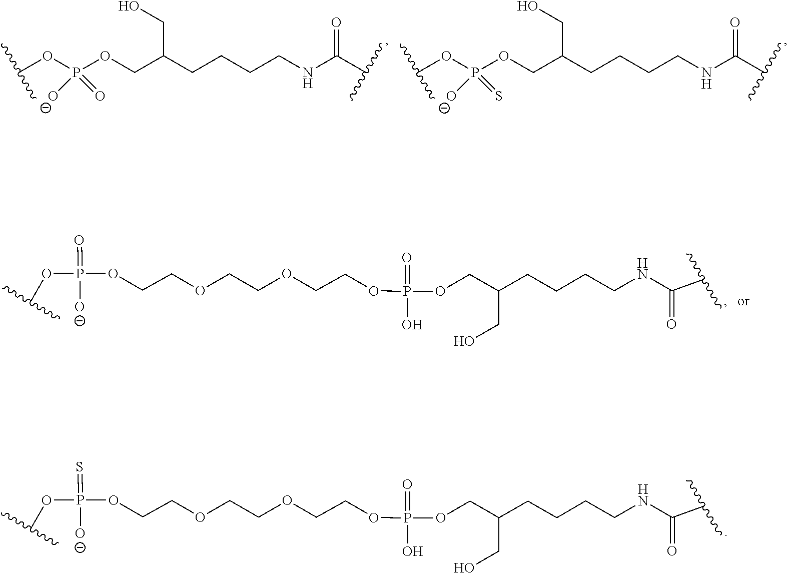 [SMILES: *OP(=O)([O-])OCC(CO)CCCCNC(*)=O ; *OP([O-])(=S)OCC(CO)CCCCNC(*)=O ; *OP(=O)([O-])OCCOCCOCCOP(=O)(O)OCC(CO)CCCCNC(*)=O ; *OP([O-])(=S)OCCOCCOCCOP(=O)(O)OCC(CO)CCCCNC(*)=O]

and is attached to a 3′ carbon of the oligonucleotide.
In embodiments, -L^{1A}-L^{1B}- is independently

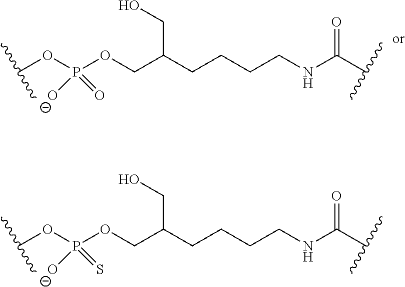 [SMILES: *OP(=O)([O-])OCC(CO)CCCCNC(*)=O ; *OP([O-])(=S)OCC(CO)CCCCNC(*)=O]

that is attached to a 3′ carbon of the oligonucleotide. In embodiments, -L^{1A}-L^{1B}- is independently

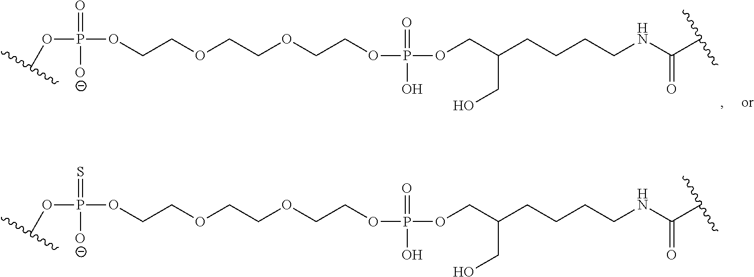 [SMILES: *OP(=O)([O-])OCCOCCOCCOP(=O)(O)OCC(CO)CCCCNC(*)=O ; *OP([O-])(=S)OCCOCCOCCOP(=O)(O)OCC(CO)CCCCNC(*)=O]

that is attached to a 3′ carbon of the oligonucleotide.

In embodiments, -L^{1A}-L^{1B}- is independently

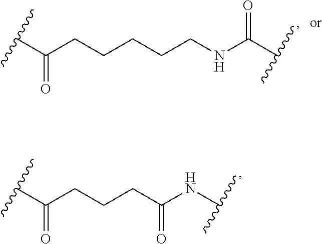 [SMILES: *C(=O)CCCCCNC(*)=O ; *NC(=O)CCCC(*)=O]

and is attached to a 3′ nitrogen of the oligonucleotide (e.g., the 3′ nitrogen of a morpholino moiety).
In embodiments, -L^{1A}-L^{1B}- is independently

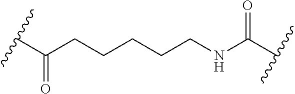 [SMILES: *C(=O)CCCCCNC(*)=O]

attached to a 3′ nitrogen of the oligonucleotide (e.g., the 3′ nitrogen of a morpholino moiety). In embodiments, -L^{1A}-L^{1B}- is independently

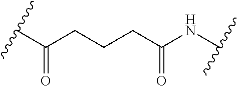 [SMILES: *NC(=O)CCCC(*)=O]

that is attached to a 3′ nitrogen of the oligonucleotide (e.g., the 3′ nitrogen of a morpholino moiety).

In embodiments, an -L^{1A}-L^{1B}- is independently

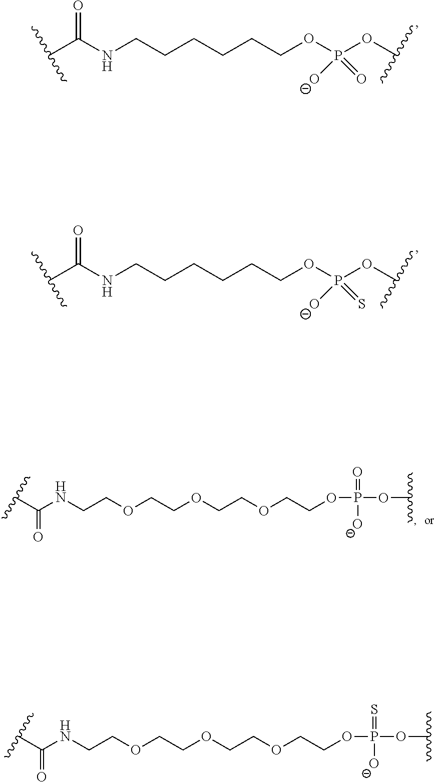 [SMILES: *OP(=O)([O-])OCCCCCCNC(*)=O ; *OP([O-])(=S)OCCCCCCNC(*)=O ; *OP(=O)([O-])OCCOCCOCCOCCNC(*)=O ; *OP([O-])(=S)OCCOCCOCCOCCNC(*)=O]

and is attached to a 5′ carbon of the oligonucleotide. In embodiments, an -L^{1A}-L^{1B}- is independently

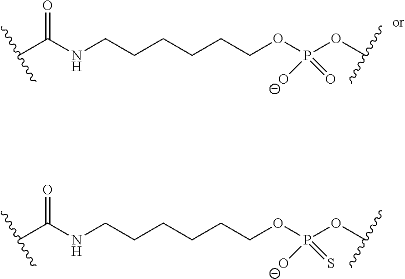 [SMILES: *OP(=O)([O-])OCCCCCCNC(*)=O ; *OP([O-])(=S)OCCCCCCNC(*)=O]

that is attached to a 5′ carbon of the oligonucleotide. In embodiments, an -L^{1A}-L^{1B}- is independently

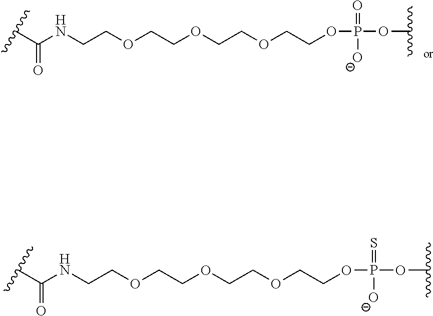 [SMILES: *OP(=O)([O-])OCCOCCOCCOCCNC(*)=O ; *OP([O-])(=S)OCCOCCOCCOCCNC(*)=O]

that is attached to a 5′ carbon of the oligonucleotide.

In embodiments where the oligonucleotide includes a morpholino moiety, L^{1A} is independently —P(O)(N(CH₃)₂)—N— or —P(O)(N(CH₃)₂)—O—. In embodiments, L^{1B} is substituted or unsubstituted heterocycloalkyl. In embodiments, L^{1B} is substituted heterocycloalkyl. In embodiments, L^{1B} is unsubstituted heterocycloalkyl. In embodiments, L^{1B} is substituted or unsubstituted piperidinylene. In embodiments, L^{1B} is substituted piperidinylene. In embodiments, L^{1B} is unsubstituted piperidinylene. In embodiments, L^{1B} is substituted or unsubstituted piperazinylene. In embodiments, L^{1B} is substituted piperazinylene. In embodiments, L^{1B} is unsubstituted piperazinylene. In embodiments, an -L^{1A}-L^{1B}- is independently

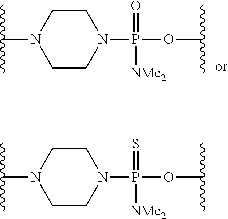 [SMILES: *OP(=O)(N(C)C)N1CCN(*)CC1 ; *OP(=S)(N(C)C)N1CCN(*)CC1]

is attached to a 6′ carbon of the oligonucleotide (e.g., the 6′ carbon of a morpholino moiety). In embodiments, an -L^{1A}-L^{1B}- is independently

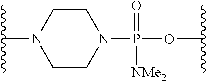 [SMILES: *OP(=O)(N(C)C)N1CCN(*)CC1]

that is attached to a 6′ carbon of the oligonucleotide (e.g., the 6′ carbon of a morpholino moiety). In embodiments, an -L^{1A}-L^{1B}- is independently

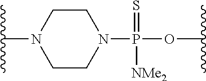 [SMILES: *OP(=S)(N(C)C)N1CCN(*)CC1]

that is attached to a 6′ carbon of the oligonucleotide (e.g., the 6′ carbon of a morpholino moiety).

In embodiments, an -L^{1A}-L^{1B}- is independently is attached to a nucleobase of the oligonucleotide. In embodiments, an -L^{1A}-L^{1B}- is independently

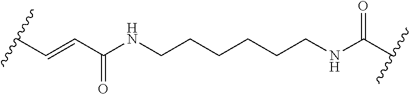 [SMILES: */C=C/C(=O)NCCCCCCNC(*)=O]

and is attached to a nucleobase of the oligonucleotide.

In embodiments, L^{1C} is independently substituted or unsubstituted alkylene, or substituted or unsubstituted heteroalkylene; L^{1D} is independently a bond, substituted or unsubstituted alkylene, substituted or unsubstituted heteroalkylene, substituted or unsubstituted arylene, or substituted or unsubstituted heteroarylene; and L^{1E} is independently a bond, substituted or unsubstituted heteroalkylene, or —NHC(O)—.

In embodiments, L^{1C} is independently substituted or unsubstituted alkylene, or substituted or unsubstituted heteroalkylene. In embodiments, L^{1C} is independently substituted or unsubstituted alkylene. In embodiments, L^{1C} is independently substituted or unsubstituted C₁-C₁₀ alkylene, or substituted or unsubstituted 2 to 10 membered heteroalkylene. In embodiments, L^{1C} is independently substituted or unsubstituted C₁-C₁₀ alkylene. In embodiments, L^{1C} is independently substituted C₁-C₁₀ alkylene. In embodiments, L^{1C} is independently unsubstituted C₁-C₁₀ alkylene. In embodiments, L^{1C} is independently substituted or unsubstituted C₁-C₈ alkylene. In embodiments, L^{1C} is independently substituted C₁-C₈ alkylene. In embodiments, L^{1C} is independently unsubstituted C₁-C₈ alkylene. In embodiments, L^{1C} is independently substituted or unsubstituted C₃-C₈ alkylene. In embodiments, L^{1C} is independently substituted C₃-C₈ alkylene. In embodiments, L^{1C} is independently unsubstituted C₃-C₈ alkylene. In embodiments, L^{1C} is independently substituted or unsubstituted C₃-C₇ alkylene. In embodiments, L^{1C} is independently substituted C₃-C₇ alkylene. In embodiments, L^{1C} is independently unsubstituted C₃-C₇ alkylene.

In embodiments, L^{1C} is independently R^{1C}-substituted or unsubstituted alkylene. In embodiments, L^{1C} is independently R^{1C}-substituted or unsubstituted C₁-C₁₀ alkylene. In embodiments, L^{1C} is independently R^{1C}-substituted C₁-C₁₀ alkylene. In embodiments, L^{1C} is independently unsubstituted C₁-C₁₀ alkylene. In embodiments, L^{1C} is independently R^{1C}-substituted or unsubstituted C₁-C₈ alkylene. In embodiments, L^{1C} is independently R^{1C}-substituted C₁-C₈ alkylene. In embodiments, L^{1C} is independently unsubstituted C₁-C₈ alkylene. In embodiments, L^{1C} is independently R^{1C}-substituted or unsubstituted C₃-C₈ alkylene. In embodiments, L^{1C} is independently R^{1C}-substituted C₃-C₈ alkylene. In embodiments, L^{1C} is independently unsubstituted C₃-C₈ alkylene. In embodiments, L^{1C} is independently R^{1C}-substituted or unsubstituted C₃-C₇ alkylene. In embodiments, L^{1C} is independently R^{1C}-substituted C₃-C₇ alkylene. In embodiments, L^{1C} is independently unsubstituted C₃-C₇ alkylene.

In embodiments, L^{1C} is independently substituted or unsubstituted heteroalkylene. In embodiments, L^{1C} is independently substituted or unsubstituted 2 to 10 membered heteroalkylene. In embodiments, L^{1C} is independently substituted 2 to 10 membered heteroalkylene. In embodiments, L^{1C} is independently unsubstituted 2 to 10 membered heteroalkylene. In embodiments, L^{1C} is independently substituted or unsubstituted 2 to 8 membered heteroalkylene. In embodiments, L^{1C} is independently substituted 2 to 8 membered heteroalkylene. In embodiments, L^{1C} is independently unsubstituted 2 to 8 membered heteroalkylene. In embodiments, L^{1C} is independently substituted or unsubstituted 5 to 8 membered heteroalkylene. In embodiments, L^{1C} is independently substituted 5 to 8 membered heteroalkylene. In embodiments, L^{1C} is independently unsubstituted 5 to 8 membered heteroalkylene.

In embodiments, L^{1C} is independently R^{1C}-substituted or unsubstituted heteroalkylene. In embodiments, L^{1C} is independently R^{1C}-substituted or unsubstituted 2 to 10 membered heteroalkylene. In embodiments, L^{1C} is independently R^{1C}-substituted 2 to 10 membered heteroalkylene. In embodiments, L^{1C} is independently unsubstituted 2 to 10 membered heteroalkylene. In embodiments, L^{1C} is independently R^{1C}-substituted or unsubstituted 2 to 8 membered heteroalkylene. In embodiments, L^{1C} is independently R^{1C}-substituted 2 to 8 membered heteroalkylene. In embodiments, L^{1C} is independently unsubstituted 2 to 8 membered heteroalkylene. In embodiments, L^{1C} is independently R^{1C}-substituted or unsubstituted 5 to 8 membered heteroalkylene. In embodiments, L^{1C} is independently R^{1C}-substituted 5 to 8 membered heteroalkylene. In embodiments, L^{1C} is independently unsubstituted 5 to 8 membered heteroalkylene.

R^{1C} is independently substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, L^{1D} is independently a bond, substituted or unsubstituted alkylene, or substituted or unsubstituted heteroalkylene. In embodiments, L^{1D} is independently a bond.

In embodiments, L^{1D} is independently substituted or unsubstituted alkylene, or substituted or unsubstituted heteroalkylene. In embodiments, L^{1D} is independently substituted or unsubstituted alkylene. In embodiments, L^{1D} is independently substituted or unsubstituted C₁-C₁₀ alkylene, or substituted or unsubstituted 2 to 10 membered heteroalkylene. In embodiments, L^{1D} is independently substituted or unsubstituted C₁-C₁₀ alkylene. In embodiments, L^{1D} is independently substituted C₁-C₁₀ alkylene. In embodiments, L^{1D} is independently unsubstituted C₁-C₁₀ alkylene. In embodiments, L^{1D} is independently substituted or unsubstituted C₁-C₈ alkylene. In embodiments, L^{1D} is independently substituted C₁-C₈ alkylene. In embodiments, L^{1D} is independently unsubstituted C₁-C₈ alkylene. In embodiments, L^{1D} is independently substituted or unsubstituted C₃-C₈ alkylene. In embodiments, L^{1D} is independently substituted C₃-C₈ alkylene. In embodiments, L^{1D} is independently unsubstituted C₃-C₈ alkylene. In embodiments, L^{1D} is independently substituted or unsubstituted C₃-C₇ alkylene. In embodiments, L^{1D} is independently substituted C₃-C₇ alkylene. In embodiments, L^{1D} is independently unsubstituted C₃-C₇ alkylene.

In embodiments, L^{1D} is independently R^{1D}-substituted or unsubstituted alkylene. In embodiments, L^{1D} is independently R^{1D}-substituted or unsubstituted C₁-C₁₀ alkylene. In embodiments, L^{1D} is independently R^{1D}-substituted C₁-C₁₀ alkylene. In embodiments, L^{1D} is independently unsubstituted C₁-C₁₀ alkylene. In embodiments, L^{1D} is independently R^{1D}-substituted or unsubstituted C₁-C₈ alkylene. In embodiments, L^{1D} is independently R^{1D}-substituted C₁-C₈ alkylene. In embodiments, L^{1D} is independently unsubstituted C₁-C₈ alkylene. In embodiments, L^{1D} is independently R^{1D}-substituted or unsubstituted C₃-C₈ alkylene. In embodiments, L^{1D} is independently R^{1D}-substituted C₃-C₈ alkylene. In embodiments, L^{1D} is independently unsubstituted C₃-C₈ alkylene. In embodiments, L^{1D} is independently R^{1D}-substituted or unsubstituted C₃-C₇ alkylene. In embodiments, L^{1D} is independently R^{1D}-substituted C₃-C₇ alkylene. In embodiments, L^{1D} is independently unsubstituted C₃-C₇ alkylene.

In embodiments, L^{1D} is independently substituted or unsubstituted heteroalkylene. In embodiments, L^{1D} is independently substituted or unsubstituted 2 to 10 membered heteroalkylene. In embodiments, L^{1D} is independently substituted 2 to 10 membered heteroalkylene. In embodiments, L^{1D} is independently unsubstituted 2 to 10 membered heteroalkylene. In embodiments, L^{1D} is independently substituted or unsubstituted 2 to 8 membered heteroalkylene. In embodiments, L^{1D} is independently substituted 2 to 8 membered heteroalkylene. In embodiments, L^{1D} is independently unsubstituted 2 to 8 membered heteroalkylene. In embodiments, L^{1D} is independently substituted or unsubstituted 5 to 8 membered heteroalkylene. In embodiments, L^{1D} is independently substituted 5 to 8 membered heteroalkylene. In embodiments, L^{1D} is independently unsubstituted 5 to 8 membered heteroalkylene.

In embodiments, L^{1D} is independently R^{1D}-substituted or unsubstituted heteroalkylene. In embodiments, L^{1D} is independently R^{1D}-substituted or unsubstituted 2 to 10 membered heteroalkylene. In embodiments, L^{1D} is independently R^{1D}-substituted 2 to 10 membered heteroalkylene. In embodiments, L^{1D} is independently unsubstituted 2 to 10 membered heteroalkylene. In embodiments, L^{1D} is independently R^{1D}-substituted or unsubstituted 2 to 8 membered heteroalkylene. In embodiments, L^{1D} is independently R^{1D}-substituted 2 to 8 membered heteroalkylene. In embodiments, L^{1D} is independently unsubstituted 2 to 8 membered heteroalkylene. In embodiments, R^{1D}-substituted or unsubstituted 5 to 8 membered heteroalkylene. In embodiments, L^{1D} is independently R^{1D}-substituted 5 to 8 membered heteroalkylene. In embodiments, L^{1D} is independently unsubstituted 5 to 8 membered heteroalkylene.

In embodiments, L^{1D} is independently substituted or unsubstituted arylene. In embodiments, L^{1D} is independently substituted or unsubstituted arylene (e.g., C₆-C₁₂, C₆-C₁₀, or phenyl). In embodiments, L^{1D} is independently substituted arylene (e.g., C₆-C₁₂, C₆-C₁₀, or phenyl). In embodiments, L^{1D} is independently unsubstituted arylene (e.g., C₆-C₁₂, C₆-C₁₀, or phenyl). In embodiments, L^{1D} is independently substituted or unsubstituted C₆-C₁₂ arylene. In embodiments, L^{1D} is independently substituted C₆-C₁₂ arylene. In embodiments, L^{1D} is independently unsubstituted C₆-C₁₂ arylene. In embodiments, L^{1D} is independently substituted or unsubstituted C₆-C₁₀ arylene. In embodiments, L^{1D} is independently substituted C₆-C₁₀ arylene. In embodiments, L^{1D} is independently unsubstituted C₆-C₁₀ arylene. In embodiments, L^{1D} is independently substituted or unsubstituted phenylene. In embodiments, L^{1D} is independently substituted phenylene. In embodiments, L^{1D} is independently unsubstituted phenylene. In embodiments, L^{1D} is independently substituted or unsubstituted biphenylene. In embodiments, L^{1D} is independently substituted biphenylene. In embodiments, L^{1D} is independently unsubstituted biphenylene. In embodiments, L^{1D} is independently substituted or unsubstituted naphthylene. In embodiments, L^{1D} is independently substituted naphthylene. In embodiments, L^{1D} is independently unsubstituted naphthylene.

In embodiments, L^{1D} is independently R^{1D}-substituted or unsubstituted arylene (e.g., C₆-C₁₂, C₆-C₁₀, or phenyl). In embodiments, L^{1D} is independently R^{1D}-substituted arylene (e.g., C₆-C₁₂, C₆-C₁₀, or phenyl). In embodiments, L^{1D} is independently unsubstituted arylene (e.g., C₆-C₁₂, C₆-C₁₀, or phenyl). In embodiments, L^{1D} is independently R^{1D}-substituted or unsubstituted C₆-C₁₂ arylene. In embodiments, L^{1D} is independently R^{1D}-substituted C₆-C₁₂ arylene. In embodiments, L^{1D} is independently unsubstituted C₆-C₁₂ arylene. In embodiments, L^{1D} is independently R^{1D}-substituted or unsubstituted C₆-C₁₀ arylene. In embodiments, L^{1D} is independently R^{1D}-substituted C₆-C₁₀ arylene. In embodiments, L^{1D} is independently unsubstituted C₆-C₁₀ arylene. In embodiments, L^{1D} is independently R^{1D}-substituted or unsubstituted phenylene. In embodiments, L^{1D} is independently R^{1D}-substituted phenylene. In embodiments, L^{1D} is independently unsubstituted phenylene. In embodiments, L^{1D} is independently R^{1D}-substituted or unsubstituted biphenylene. In embodiments, L^{1D} is independently R^{1D}-substituted biphenylene. In embodiments, L^{1D} is independently unsubstituted biphenylene. In embodiments, L^{1D} is independently R^{1D}-substituted or unsubstituted naphthylene. In embodiments, L^{1D} is independently R^{1D}-substituted naphthylene. In embodiments, L^{1D} is independently unsubstituted naphthylene.

In embodiments, L^{1D} is independently substituted or unsubstituted heteroarylene. In embodiments, L^{1D} is independently substituted heteroarylene. In embodiments, L^{1D} is independently unsubstituted heteroarylene. In embodiments, L^{1D} is independently substituted or unsubstituted heteroarylene (e.g., 5 to 12 membered, 5 to 10 membered, 5 to 9 membered, or 5 to 6 membered). In embodiments, L^{1D} is independently substituted heteroarylene (e.g., 5 to 12 membered, 5 to 10 membered, 5 to 9 membered, or 5 to 6 membered). In embodiments, L^{1D} is independently unsubstituted heteroarylene (e.g., 5 to 12 membered, 5 to 10 membered, 5 to 9 membered, or 5 to 6 membered). In embodiments, L^{1D} is independently substituted or unsubstituted 5 to 12 membered heteroarylene. In embodiments, L^{1D} is independently substituted 5 to 12 membered heteroarylene. In embodiments, L^{1D} is independently unsubstituted 5 to 12 membered heteroarylene. In embodiments, L^{1D} is independently substituted or unsubstituted 5 to 10 membered heteroarylene. In embodiments, L^{1D} is independently substituted 5 to 10 membered heteroarylene. In embodiments, L^{1D} is independently unsubstituted 5 to 10 membered heteroarylene. In embodiments, L^{1D} is independently substituted or unsubstituted 5 to 9 membered heteroarylene. In embodiments, L^{1D} is independently substituted 5 to 9 membered heteroarylene. In embodiments, L^{1D} is independently unsubstituted 5 to 9 membered heteroarylene. In embodiments, L^{1D} is independently substituted or unsubstituted 5 to 6 membered heteroarylene. In embodiments, L^{1D} is independently substituted 5 to 6 membered heteroarylene. In embodiments, L^{1D} is independently unsubstituted 5 to 6 membered heteroarylene.

R^{1D} is independently substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, L^{1E} is independently a bond, substituted or unsubstituted 2 to 10 membered heteroalkylene, or —NHC(O)—. In embodiments, L^{1E} is independently a bond. In embodiments, L^{1E} is independently —NHC(O)—.

In embodiments, L^{1E} is independently substituted or unsubstituted heteroalkylene. In embodiments, L^{1E} is independently substituted or unsubstituted 2 to 10 membered heteroalkylene. In embodiments, L^{1E} is independently substituted 2 to 10 membered heteroalkylene. In embodiments, L^{1E} is independently unsubstituted 2 to 10 membered heteroalkylene. In embodiments, L^{1E} is independently substituted or unsubstituted 2 to 8 membered heteroalkylene. In embodiments, L^{1E} is independently substituted 2 to 8 membered heteroalkylene. In embodiments, L^{1E} is independently unsubstituted 2 to 8 membered heteroalkylene. In embodiments, L^{1E} is independently substituted or unsubstituted 5 to 8 membered heteroalkylene. In embodiments, L^{1E} is independently substituted 5 to 8 membered heteroalkylene. In embodiments, L^{1E} is independently unsubstituted 5 to 8 membered heteroalkylene.

In embodiments, L^{1E} is independently R^{1E}-substituted or unsubstituted heteroalkylene. In embodiments, L^{1E} is independently R^{1E}-substituted or unsubstituted 2 to 10 membered heteroalkylene. In embodiments, L^{1E} is independently R^{1E}-substituted 2 to 10 membered heteroalkylene. In embodiments, L^{1E} is independently unsubstituted 2 to 10 membered heteroalkylene. In embodiments, L^{1E} is independently R^{1E}-substituted or unsubstituted 2 to 8 membered heteroalkylene. In embodiments, L^{1E} is independently R^{1E}-substituted 2 to 8 membered heteroalkylene. In embodiments, L^{1E} is independently unsubstituted 2 to 8 membered heteroalkylene. In embodiments, L^{1E} is independently R^{1E}-substituted or unsubstituted 5 to 8 membered heteroalkylene. In embodiments, L^{1E} is independently R^{1E}-substituted 5 to 8 membered heteroalkylene. In embodiments, L^{1E} is independently unsubstituted 5 to 8 membered heteroalkylene.

R^{1E} is independently substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, L^{1C} is independently R^{1C}-substituted or unsubstituted C₁-C₇ alkylene, or R^{1C}-substituted or unsubstituted 5 to 8 membered heteroalkylene; L^{1D} is independently a bond, R^{1D}-substituted or unsubstituted C₁-C₇ alkylene, or R^{1D}-substituted or unsubstituted 5 to 8 membered heteroalkylene; and L^{1E} is independently a bond, R^{1E}-substituted or unsubstituted 5 to 8 membered heteroalkylene, or —NHC(O)—.

In embodiments, L^{1C} is independently R^{1C}-substituted or unsubstituted C₁-C₇ alkylene. In embodiments, L^{1C} is independently R^{1C}-substituted or unsubstituted 5 to 8 membered heteroalkylene. In embodiments, L^{1D} is independently a bond. In embodiments, L^{1D} is independently R^{1D}-substituted or unsubstituted C₁-C₇ alkylene. In embodiments, L^{1E} is independently a bond. In embodiments, L^{1E} is independently R^{1E}-substituted or unsubstituted 5 to 8 membered heteroalkylene. In embodiments, L^{1E} is independently —NHC(O)—.

In embodiments, R^{1C} is independently oxo, or -L^{8C}-L^{2C}-R^{8C}. In embodiments, R^{1C} is independently oxo. In embodiments, R^{1C} is independently -L^{8C}-L^{2C}-R^{8C}. L^{8C} is independently a bond, substituted or unsubstituted C₁-C₆ alkylene, or substituted or unsubstituted 2 to 6 membered heteroalkylene. L^{8C} is independently a bond, substituted or unsubstituted C₁-C₆ alkylene, or substituted or unsubstituted 2 to 6 membered heteroalkylene. L^{2C} is independently a bond, or an unsubstituted alkylene. R^{8C} is independently hydrogen, substituted or unsubstituted alkyl, or substituted or unsubstituted heteroalkyl.

In embodiments, R^{1D} is independently oxo, or -L^{8D}-L^{2D}-R^{8D}. In embodiments, R^{1D} is independently oxo. In embodiments, R^{1D} is independently -L^{8D}-L^{2D}-R^{8D}. L^{8D} is independently a bond, substituted or unsubstituted C₁-C₆ alkylene, or substituted or unsubstituted 2 to 6 membered heteroalkylene. L^{8D} is independently a bond, substituted or unsubstituted C₁-C₆ alkylene, or substituted or unsubstituted 2 to 6 membered heteroalkylene. L^{2D} is independently a bond, or an unsubstituted alkylene. R^{8D} is independently hydrogen, substituted or unsubstituted alkyl, or substituted or unsubstituted heteroalkyl.

In embodiments, R^{1E} is independently oxo, or -L^{8E}-L^{2E}-R^{8E}. In embodiments, R^{1E} is independently oxo. In embodiments, R^{1E} is independently -L^{8E}-L^{2E}-R^{8E}. L^{8E} is independently a bond, substituted or unsubstituted C₁-C₆ alkylene, or substituted or unsubstituted 2 to 6 membered heteroalkylene. L^{8E} is independently a bond, substituted or unsubstituted C₁-C₆ alkylene, or substituted or unsubstituted 2 to 6 membered heteroalkylene. L^{2E} is independently a bond, or an unsubstituted alkylene. R^{8E} is independently hydrogen, substituted or unsubstituted alkyl, or substituted or unsubstituted heteroalkyl.

In embodiments, the half-life extension motif has the structure of:

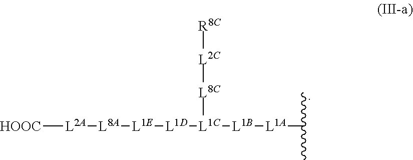

L^{8A} is independently a bond, substituted or unsubstituted alkylene, or substituted or unsubstituted heteroalkylene. L^{2A} is independently a bond, or an unsubstituted alkylene. L^{1A}, L^{1B}, L^{1C}, L^{1D}, L^{1E}, L^{2C}, L^{8C}, and R^{8C} are as described above.

In embodiments, the half-life extension motif has the structure of:

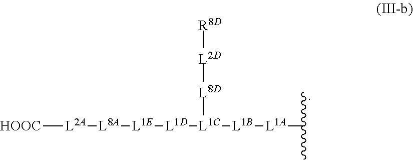

L^{1A}, L^{1B}, L^{1C}, L^{1D}, L^{1E}, L^{2A}, L^{2D}, L^{8A}, L^{8D}, and R^{8D} are as described above.

In embodiments, the half-life extension motif has the structure of:

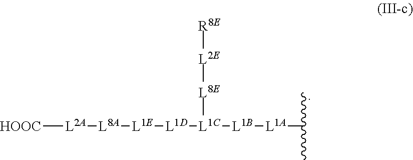

L^{1A}, L^{1B}, L^{1C}, L^{1D}, L^{1E}, L^{2A}, L^{2E}, L^{8A}, L^{8E}, and R^{8E} are as described above.

In embodiments, the half-life extension motif has the structure of:

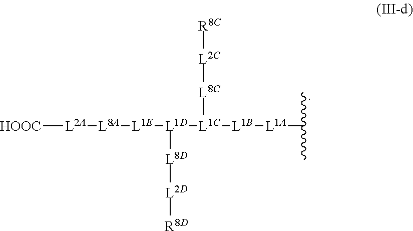

L^{1A}, L^{1B}, L^{1C}, L^{1D}, L^{1E}, L^{2A}, L^{2C}, L^{2D}, L^{8A}, L^{8C}, L^{8D}, R^{8C} and R^{8D} are as described above.

In embodiments, the half-life extension motif has the structure of:

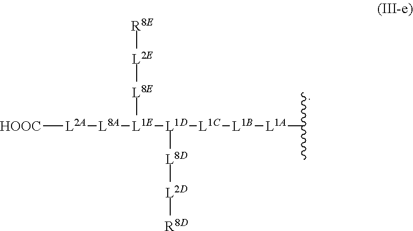

L^{1A}, L^{1B}, L^{1C}, L^{1D}, L^{1E}, L^{2A}, L^{2D}, L^{2E}, L^{8A}, L^{8D}, L^{8E}, R^{8D} and R^{8E} are as described above.

In embodiments, the half-life extension motif has the structure of:

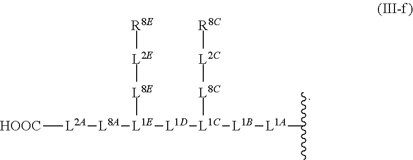

L^{1A}, L^{1B}, L^{1C}, L^{1D}, L^{1E}, L^{2A}, L^{2C}, L^{2E}, L^{8A}, L^{8C}, L^{8E}, R^{8C} and R^{8E} are as described above.

In embodiments, the half-life extension motif has the structure of:

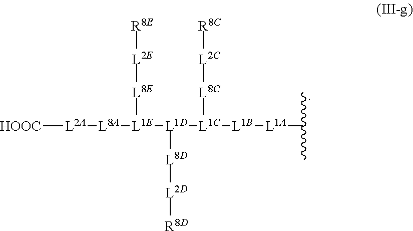

L^{1A}, L^{1B}, L^{1C}, L^{1D}, L^{1E}, L^{2A}, L^{2C}, L^{2D}, L^{2E}, L^{8A}, L^{8C}, L^{8D}, L^{2E}, R^{8C}, R^{8D} and R^{8E} are as described above.

In embodiments, L^{8A} is independently a bond. In embodiments, L^{8A} is independently substituted or unsubstituted alkylene, or substituted or unsubstituted heteroalkylene. In embodiments, L^{8A} is independently substituted or unsubstituted alkylene. In embodiments, L^{8A} is independently substituted or unsubstituted C₁-C₁₀ alkylene, or substituted or unsubstituted 2 to 10 membered heteroalkylene. In embodiments, L^{8A} is independently substituted or unsubstituted C₁-C₁₀ alkylene. In embodiments, L^{8A} is independently substituted C₁-C₁₀ alkylene. In embodiments, L^{8A} is independently unsubstituted C₁-C₁₀ alkylene. In embodiments, L^{8A} is independently substituted or unsubstituted C₁-C₈ alkylene. In embodiments, L^{8A} is independently substituted C₁-C₈ alkylene. In embodiments, L^{8A} is independently unsubstituted C₁-C₈ alkylene. In embodiments, L^{8A} is independently substituted or unsubstituted C₃-C₈ alkylene. In embodiments, L^{8A} is independently substituted C₃-C₈ alkylene. In embodiments, L^{8A} is independently unsubstituted C₃-C₈ alkylene. In embodiments, L^{8A} is independently substituted or unsubstituted C₃-C₇ alkylene. In embodiments, L^{8A} is independently substituted C₃-C₇ alkylene. In embodiments, L^{8A} is independently unsubstituted C₃-C₇ alkylene.

In embodiments, L^{8A} is independently R^{8A}-substituted or unsubstituted alkylene. In embodiments, L^{8A} is independently R^{8A}-substituted or unsubstituted C₁-C₁₀ alkylene. In embodiments, L^{8A} is independently R^{8A}-substituted C₁-C₁₀ alkylene. In embodiments, L^{8A} is independently unsubstituted C₁-C₁₀ alkylene. In embodiments, L^{8A} is independently R^{8A}-substituted or unsubstituted C₁-C₈ alkylene. In embodiments, L^{8A} is independently R^{8A}-substituted C₁-C₈ alkylene. In embodiments, L^{8A} is independently unsubstituted C₁-C₈ alkylene. In embodiments, L^{8A} is independently R^{8A}-substituted or unsubstituted C₃-C₈ alkylene. In embodiments, L^{8A} is independently R^{8A}-substituted C₃-C₈ alkylene. In embodiments, L^{8A} is independently unsubstituted C₃-C₈ alkylene. In embodiments, L^{8A} is independently R^{8A}-substituted or unsubstituted C₃-C₇ alkylene. In embodiments, L^{8A} is independently R^{8A}-substituted C₃-C₇ alkylene. In embodiments, L^{8A} is independently unsubstituted C₃-C₇ alkylene.

In embodiments, L^{8A} is independently substituted or unsubstituted heteroalkylene. In embodiments, L^{8A} is independently substituted or unsubstituted 2 to 10 membered heteroalkylene. In embodiments, L^{8A} is independently substituted 2 to 10 membered heteroalkylene. In embodiments, L^{8A} is independently unsubstituted 2 to 10 membered heteroalkylene. In embodiments, L^{8A} is independently substituted or unsubstituted 2 to 8 membered heteroalkylene. In embodiments, L^{8A} is independently substituted 2 to 8 membered heteroalkylene. In embodiments, L^{8A} is independently unsubstituted 2 to 8 membered heteroalkylene. In embodiments, L^{8A} is independently substituted or unsubstituted 5 to 8 membered heteroalkylene. In embodiments, L^{8A} is independently substituted 5 to 8 membered heteroalkylene. In embodiments, L^{8A} is independently unsubstituted 5 to 8 membered heteroalkylene.

In embodiments, L^{8A} is independently R^{8A}-substituted or unsubstituted heteroalkylene. In embodiments, L^{8A} is independently R^{8A}-substituted or unsubstituted 2 to 10 membered heteroalkylene. In embodiments, L^{8A} is independently R^{8A}-substituted 2 to 10 membered heteroalkylene. In embodiments, L^{8A} is independently unsubstituted 2 to 10 membered heteroalkylene. In embodiments, L^{8A} is independently R^{8A}-substituted or unsubstituted 2 to 8 membered heteroalkylene. In embodiments, L^{8A} is independently R^{8A}-substituted 2 to 8 membered heteroalkylene. In embodiments, L^{8A} is independently unsubstituted 2 to 8 membered heteroalkylene. In embodiments, R^{8A}-substituted or unsubstituted 5 to 8 membered heteroalkylene. In embodiments, L^{8A} is independently R^{8A}-substituted 5 to 8 membered heteroalkylene. In embodiments, L^{8A} is independently unsubstituted 5 to 8 membered heteroalkylene.

In embodiments, L^{2A} is independently an unsubstituted C₂-C₂₄ alkylene. In embodiments, L^{2A} is independently an unsubstituted C₂-C₂₂ alkylene. In embodiments, L^{2A} is independently an unsubstituted C₅-C₂₂ alkylene. In embodiments, L^{2A} is independently an unsubstituted C₁₀-C₂₂ alkylene. In embodiments, L^{2A} is independently an unsubstituted C₁₂-C₂₂ alkylene. In embodiments, L^{2A} is independently an unsubstituted C₁₀-C₂₀ alkylene. In embodiments, L^{2A} is independently an unsubstituted C₁₂-C₂₀ alkylene. In embodiments, L^{2A} is independently an unsubstituted C₁₀-C₁₈ alkylene. In embodiments, L^{2A} is independently an unsubstituted C₁₂-C₁₈ alkylene. In embodiments, L^{2A} is independently an unsubstituted C₁₀-C₁₆ alkylene. In embodiments, L^{2A} is independently an unsubstituted C₁₂-C₁₆ alkylene. In embodiments, L^{2A} is independently an unsubstituted C₁₄-C₁₆ alkylene. In embodiments, L^{2A} is independently an unsubstituted C₁₄-C₁₅ alkylene. In embodiments, L^{2A} is independently an unsubstituted C₁₄ alkylene. In embodiments, L^{2A} is independently an unsubstituted C₁₅ alkylene. In embodiments, L^{2A} is independently an unsubstituted C₁₆ alkylene.

In embodiments, L^{2A} is independently an unsubstituted unbranched C₂-C₂₄ alkylene. In embodiments, L^{2A} is independently an unsubstituted unbranched C₂-C₂₂ alkylene. In embodiments, L^{2A} is independently an unsubstituted unbranched C₅-C₂₂ alkylene. In embodiments, L^{2A} is independently an unsubstituted unbranched C₁₀-C₂₂ alkylene. In embodiments, L^{2A} is independently an unsubstituted unbranched C₁₂-C₂₂ alkylene. In embodiments, L^{2A} is independently an unsubstituted unbranched C₁₀-C₂₀ alkylene. In embodiments, L^{2A} is independently an unsubstituted unbranched C₁₂-C₂₀ alkylene. In embodiments, L^{2A} is independently an unsubstituted unbranched C₁₀-C₁₈ alkylene. In embodiments, L^{2A} is independently an unsubstituted unbranched C₁₂-C₁₈ alkylene. In embodiments, L^{2A} is independently an unsubstituted unbranched C₁₀-C₁₆ alkylene. In embodiments, L^{2A} is independently an unsubstituted unbranched C₁₂-C₁₆ alkylene. In embodiments, L^{2A} is independently an unsubstituted unbranched C₁₄-C₁₆ alkylene. In embodiments, L^{2A} is independently an unsubstituted unbranched C₁₄-C₁₅ alkylene. In embodiments, L^{2A} is independently an unsubstituted unbranched C₁₄ alkylene. In embodiments, L^{2A} is independently an unsubstituted unbranched C₁₅ alkylene. In embodiments, L^{2A} is independently an unsubstituted unbranched C₁₆ alkylene.

In embodiments, L^{2A} is independently an unsubstituted ununbranched saturated C₂-C₂₄ alkylene. In embodiments, L^{2A} is independently an unsubstituted ununbranched saturated C₂-C₂₂ alkylene. In embodiments, L^{2A} is independently an unsubstituted ununbranched saturated C₅-C₂₂ alkylene. In embodiments, L^{2A} is independently an unsubstituted ununbranched saturated C₁₀-C₂₂ alkylene. In embodiments, L^{2A} is independently an unsubstituted ununbranched saturated C₁₂-C₂₂ alkylene. In embodiments, L^{2A} is independently an unsubstituted ununbranched saturated C₁₀-C₂₀ alkylene. In embodiments, L^{2A} is independently an unsubstituted ununbranched saturated C₁₂-C₂₀ alkylene. In embodiments, L^{2A} is independently an unsubstituted ununbranched saturated C₁₀-C₁₈ alkylene. In embodiments, L^{2A} is independently an unsubstituted ununbranched saturated C₁₂-C₁₈ alkylene. In embodiments, L^{2A} is independently an unsubstituted ununbranched saturated C₁₀-C₁₆ alkylene. In embodiments, L^{2A} is independently an unsubstituted ununbranched saturated C₁₂-C₁₆ alkylene. In embodiments, L^{2A} is independently an unsubstituted ununbranched saturated C₁₄-C₁₆ alkylene. In embodiments, L^{2A} is independently an unsubstituted ununbranched saturated C₁₄-C₁₅ alkylene. In embodiments, L^{2A} is independently an unsubstituted ununbranched saturated C₁₄ alkylene. In embodiments, L^{2A} is independently an unsubstituted ununbranched saturated C₁₅ alkylene. In embodiments, L^{2A} is independently an unsubstituted ununbranched saturated C₁₆ alkylene.

In embodiments, L^{2A} is independently an unsubstituted ununbranched unsaturated C₂-C₂₄ alkylene. In embodiments, L^{2A} is independently an unsubstituted ununbranched unsaturated C₂-C₂₂ alkylene. In embodiments, L^{2A} is independently an unsubstituted ununbranched unsaturated C₅-C₂₂ alkylene. In embodiments, L^{2A} is independently an unsubstituted ununbranched unsaturated C₁₀-C₂₂ alkylene. In embodiments, L^{2A} is independently an unsubstituted ununbranched unsaturated C₁₂-C₂₂ alkylene. In embodiments, L^{2A} is independently an unsubstituted ununbranched unsaturated C₁₀-C₂₀ alkylene. In embodiments, L^{2A} is independently an unsubstituted ununbranched unsaturated C₁₂-C₂₀ alkylene. In embodiments, L^{2A} is independently an unsubstituted ununbranched unsaturated C₁₀-C₁₈ alkylene. In embodiments, L^{2A} is independently an unsubstituted ununbranched unsaturated C₁₂-C₁₅ alkylene. In embodiments, L^{2A} is independently an unsubstituted ununbranched unsaturated C₁₀-C₁₆ alkylene. In embodiments, L^{2A} is independently an unsubstituted ununbranched unsaturated C₁₂-C₁₆ alkylene. In embodiments, L^{2A} is independently an unsubstituted ununbranched unsaturated C₁₄-C₁₆ alkylene. In embodiments, L^{2A} is independently an unsubstituted ununbranched unsaturated C₁₄-C₁₅ alkylene. In embodiments, L^{2A} is independently an unsubstituted ununbranched unsaturated C₁₄ alkylene. In embodiments, L^{2A} is independently an unsubstituted ununbranched unsaturated C₁₅ alkylene. In embodiments, L^{2A} is independently an unsubstituted ununbranched unsaturated C₁₆ alkylene. In embodiments, L^{8A} is independently a bond and L^{2A} is independently an unsubstituted C₂-C₂₂ alkylene.

In embodiments, L^{2A} is independently a bond and L^{8A} is independently an unsubstituted C₂-C₂₂ alkylene.

In embodiments, L^{8A} is independently an unsubstituted C₂-C₂₄ alkylene. In embodiments, L^{8A} is independently an unsubstituted C₂-C₂₂ alkylene. In embodiments, L^{8A} is independently an unsubstituted C₅-C₂₂ alkylene. In embodiments, L^{8A} is independently an unsubstituted C₁₀-C₂₂ alkylene. In embodiments, L^{8A} is independently an unsubstituted C₁₂-C₂₂ alkylene. In embodiments, L^{8A} is independently an unsubstituted C₁₀-C₂₀ alkylene. In embodiments, L^{8A} is independently an unsubstituted C₁₂-C₂₀ alkylene. In embodiments, L^{8A} is independently an unsubstituted C₁₀-C₁₈ alkylene. In embodiments, L^{8A} is independently an unsubstituted C₁₂-C₁₈ alkylene. In embodiments, L^{8A} is independently an unsubstituted C₁₀-C₁₆ alkylene. In embodiments, L^{8A} is independently an unsubstituted C₁₂-C₁₆ alkylene. In embodiments, L^{8A} is independently an unsubstituted C₁₄-C₁₆ alkylene. In embodiments, L^{8A} is independently an unsubstituted C₁₄-C₁₅ alkylene. In embodiments, L^{8A} is independently an unsubstituted C₁₄ alkylene. In embodiments, L^{8A} is independently an unsubstituted C₁₅ alkylene. In embodiments, L^{8A} is independently an unsubstituted C₁₆ alkylene.

In embodiments, L^{8A} is independently an unsubstituted unbranched C₂-C₂₄ alkylene. In embodiments, L^{8A} is independently an unsubstituted unbranched C₂-C₂₂ alkylene. In embodiments, L^{8A} is independently an unsubstituted unbranched C₅-C₂₂ alkylene. In embodiments, L^{8A} is independently an unsubstituted unbranched C₁₀-C₂₂ alkylene. In embodiments, L^{8A} is independently an unsubstituted unbranched C₁₂-C₂₂ alkylene. In embodiments, L^{8A} is independently an unsubstituted unbranched C₁₀-C₂₀ alkylene. In embodiments, L^{8A} is independently an unsubstituted unbranched C₁₂-C₂₀ alkylene. In embodiments, L^{8A} is independently an unsubstituted unbranched C₁₀-C₁₈ alkylene. In embodiments, L^{8A} is independently an unsubstituted unbranched C₁₂-C₁₈ alkylene. In embodiments, L^{8A} is independently an unsubstituted unbranched C₁₀-C₁₆ alkylene. In embodiments, L^{8A} is independently an unsubstituted unbranched C₁₂-C₁₆ alkylene. In embodiments, L^{8A} is independently an unsubstituted unbranched C₁₄-C₁₆ alkylene. In embodiments, L^{8A} is independently an unsubstituted unbranched C₁₄-C₁₅ alkylene. In embodiments, L^{8A} is independently an unsubstituted unbranched C₁₄ alkylene. In embodiments, L^{8A} is independently an unsubstituted unbranched C₁₅ alkylene. In embodiments, L^{8A} is independently an unsubstituted unbranched C₁₆ alkylene.

In embodiments, L^{8A} is independently an unsubstituted ununbranched saturated C₂-C₂₄ alkylene. In embodiments, L^{8A} is independently an unsubstituted ununbranched saturated C₂-C₂₂ alkylene. In embodiments, L^{8A} is independently an unsubstituted ununbranched saturated C₅-C₂₂ alkylene. In embodiments, L^{8A} is independently an unsubstituted ununbranched saturated C₁₀-C₂₂ alkylene. In embodiments, L^{8A} is independently an unsubstituted ununbranched saturated C₁₂-C₂₂ alkylene. In embodiments, L^{8A} is independently an unsubstituted ununbranched saturated C₁₀-C₂₀ alkylene. In embodiments, L^{8A} is independently an unsubstituted ununbranched saturated C₁₂-C₂₀ alkylene. In embodiments, L^{8A} is independently an unsubstituted ununbranched saturated C₁₀-C₁₈ alkylene. In embodiments, L^{8A} is independently an unsubstituted ununbranched saturated C₁₂-Cis alkylene. In embodiments, L^{8A} is independently an unsubstituted ununbranched saturated C₁₀-C₁₆ alkylene. In embodiments, L^{8A} is independently an unsubstituted ununbranched saturated C₁₂-C₁₆ alkylene. In embodiments, L^{8A} is independently an unsubstituted ununbranched saturated C₁₄-C₁₆ alkylene. In embodiments, L^{8A} is independently an unsubstituted ununbranched saturated C₁₄-C₁₅ alkylene. In embodiments, L^{8A} is independently an unsubstituted ununbranched saturated C₁₄ alkylene. In embodiments, L^{8A} is independently an unsubstituted ununbranched saturated C₁₅ alkylene. In embodiments, L^{8A} is independently an unsubstituted ununbranched saturated C₁₆ alkylene.

In embodiments, L^{8A} is independently an unsubstituted ununbranched unsaturated C₂-C₂₄ alkylene. In embodiments, L^{8A} is independently an unsubstituted ununbranched unsaturated C₂-C₂₂ alkylene. In embodiments, L^{8A} is independently an unsubstituted ununbranched unsaturated C₅-C₂₂ alkylene. In embodiments, L^{8A} is independently an unsubstituted ununbranched unsaturated C₁₀-C₂₂ alkylene. In embodiments, L^{8A} is independently an unsubstituted ununbranched unsaturated C₁₂-C₂₂ alkylene. In embodiments, L^{8A} is independently an unsubstituted ununbranched unsaturated C₁₀-C₂₀ alkylene. In embodiments, L^{8A} is independently an unsubstituted ununbranched unsaturated C₁₂-C₂₀ alkylene. In embodiments, L^{8A} is independently an unsubstituted ununbranched unsaturated C₁₀-C₁₈ alkylene. In embodiments, L^{8A} is independently an unsubstituted ununbranched unsaturated C₁₂-C₁₈ alkylene. In embodiments, L^{8A} is independently an unsubstituted ununbranched unsaturated C₁₀-C₁₆ alkylene. In embodiments, L^{8A} is independently an unsubstituted ununbranched unsaturated C₁₂-C₁₆ alkylene. In embodiments, L^{8A} is independently an unsubstituted ununbranched unsaturated C₁₄-C₁₆ alkylene. In embodiments, L^{8A} is independently an unsubstituted ununbranched unsaturated C₁₄-C₁₅ alkylene. In embodiments, L^{8A} is independently an unsubstituted ununbranched unsaturated C₁₄ alkylene. In embodiments, L^{8A} is independently an unsubstituted ununbranched unsaturated C₁₅ alkylene. In embodiments, L^{8A} is independently an unsubstituted ununbranched unsaturated C₁₆ alkylene.

In embodiments, R^{1C} is independently —NHC(O)-L^{2C}-R^{8C}; L^{2C} is independently a bond or an unsubstituted C₂-C₂₂ alkylene; and R^{8C} is independently hydrogen, C₁-C₃ alkyl, or —COOH. In embodiments, R^{1C} is independently —NHC(O)-L^{2C}-R^{8C}. In embodiments, L^{2C} is independently a bond or an unsubstituted C₂-C₂₂ alkylene. In embodiments, L^{2C} is independently a bond. In embodiments, L^{2C} is independently an unsubstituted C₂-C₂₂ alkylene. In embodiments, R^{8C} is independently hydrogen, C₁-C₃ alkyl, or —COOH. In embodiments, R^{8C} is independently hydrogen. In embodiments, R^{8C} is independently C₁-C₃ alkyl. In embodiments, R^{8C} is independently —COOH.

In embodiments, R^{1C} is independently —NHC(O)-L^{2C}-R^{8C}, L^{2C} is independently a bond, and R^{8C} is independently C₁-C₃ alkyl. In embodiments, R^{1C} is independently —NHC(O)—CH₃. In embodiments, R^{1C} is independently —NHC(O)—CH₂CH₃. In embodiments, R^{1C} is independently —NHC(O)—CH(CH₃)₂. In embodiments, R^{1C} is independently —NHC(O)—CH₂CH₂CH₃.

In embodiments, R^{1C} is independently —NHC(O)-L^{2C}-R^{8C}, L^{2C} is independently an unsubstituted C₁₀-C₂₂ alkylene, and R^{8C} is independently —COOH. In embodiments, R^{1C} is independently —NHC(O)-L^{2C}-COOH.

In embodiments, L^{2C} is independently an unsubstituted unbranched C₂-C₂₄ alkylene. In embodiments, L^{2C} is independently an unsubstituted unbranched C₂-C₂₂ alkylene. In embodiments, L^{2C} is independently an unsubstituted unbranched C₅-C₂₂ alkylene. In embodiments, L^{2C} is independently an unsubstituted unbranched C₁₀-C₂₂ alkylene. In embodiments, L^{2C} is independently an unsubstituted unbranched C₁₂-C₂₂ alkylene. In embodiments, L^{2C} is independently an unsubstituted unbranched C₁₀-C₂₀ alkylene. In embodiments, L^{2C} is independently an unsubstituted unbranched C₁₂-C₂₀ alkylene. In embodiments, L^{2C} is independently an unsubstituted unbranched C₁₀-C₁₈ alkylene. In embodiments, L^{2C} is independently an unsubstituted unbranched C₁₂-C₁₈ alkylene. In embodiments, L^{2C} is independently an unsubstituted unbranched C₁₀-C₁₆ alkylene. In embodiments, L^{2C} is independently an unsubstituted unbranched C₁₂-C₁₆ alkylene. In embodiments, L^{2C} is independently an unsubstituted unbranched C₁₄-C₁₆ alkylene. In embodiments, L^{2C} is independently an unsubstituted unbranched C₁₄-C₁₅ alkylene. In embodiments, L^{2C} is independently an unsubstituted unbranched C₁₄ alkylene. In embodiments, L^{2C} is independently an unsubstituted unbranched C₁₅ alkylene. In embodiments, L^{2C} is independently an unsubstituted unbranched C₁₆ alkylene.

In embodiments, L^{2C} is independently an unsubstituted ununbranched saturated C₂-C₂₄ alkylene. In embodiments, L^{2C} is independently an unsubstituted ununbranched saturated C₂-C₂₂ alkylene. In embodiments, L^{2C} is independently an unsubstituted ununbranched saturated C₅-C₂₂ alkylene. In embodiments, L^{2C} is independently an unsubstituted ununbranched saturated C₁₀-C₂₂ alkylene. In embodiments, L^{2C} is independently an unsubstituted ununbranched saturated C₁₂-C₂₂ alkylene. In embodiments, L^{2C} is independently an unsubstituted ununbranched saturated C₁₀-C₂₀ alkylene. In embodiments, L^{2C} is independently an unsubstituted ununbranched saturated C₁₂-C₂₀ alkylene. In embodiments, L^{2C} is independently an unsubstituted ununbranched saturated C₁₀-C₁₈ alkylene. In embodiments, L^{2C} is independently an unsubstituted ununbranched saturated C₁₂-Cis alkylene. In embodiments, L^{2C} is independently an unsubstituted ununbranched saturated C₁₀-C₁₆ alkylene. In embodiments, L^{2C} is independently an unsubstituted ununbranched saturated C₁₂-C₁₆ alkylene. In embodiments, L^{2C} is independently an unsubstituted ununbranched saturated C₁₄-C₁₆ alkylene. In embodiments, L^{2C} is independently an unsubstituted ununbranched saturated C₁₄-C₁₅ alkylene. In embodiments, L^{2C} is independently an unsubstituted ununbranched saturated C₁₄ alkylene. In embodiments, L^{2C} is independently an unsubstituted ununbranched saturated C₁₅ alkylene. In embodiments, L^{2C} is independently an unsubstituted ununbranched saturated C₁₆ alkylene.

In embodiments, L^{2C} is independently an unsubstituted ununbranched unsaturated C₂-C₂₄ alkylene. In embodiments, L^{2C} is independently an unsubstituted ununbranched unsaturated C₂-C₂₂ alkylene. In embodiments, L^{2C} is independently an unsubstituted ununbranched unsaturated C₅-C₂₂ alkylene. In embodiments, L^{2C} is independently an unsubstituted ununbranched unsaturated C₁₀-C₂₂ alkylene. In embodiments, L^{2C} is independently an unsubstituted ununbranched unsaturated C₁₂-C₂₂ alkylene. In embodiments, L^{2C} is independently an unsubstituted ununbranched unsaturated C₁₀-C₂₀ alkylene. In embodiments, L^{2C} is independently an unsubstituted ununbranched unsaturated C₁₂-C₂₀ alkylene. In embodiments, L^{2C} is independently an unsubstituted ununbranched unsaturated C₁₀-C₁₈ alkylene. In embodiments, L^{2C} is independently an unsubstituted ununbranched unsaturated C₁₂-C₁₅ alkylene. In embodiments, L^{2C} is independently an unsubstituted ununbranched unsaturated C₁₀-C₁₆ alkylene. In embodiments, L^{2C} is independently an unsubstituted ununbranched unsaturated C₁₂-C₁₆ alkylene. In embodiments, L^{2C} is independently an unsubstituted ununbranched unsaturated C₁₄-C₁₆ alkylene. In embodiments, L^{2C} is independently an unsubstituted ununbranched unsaturated C₁₄-C₁₅ alkylene. In embodiments, L^{2C} is independently an unsubstituted ununbranched unsaturated C₁₄ alkylene. In embodiments, L^{2C} is independently an unsubstituted ununbranched unsaturated C₁₅ alkylene. In embodiments, L^{2C} is independently an unsubstituted ununbranched unsaturated C₁₆ alkylene.

In embodiments, R^{1D} is independently —NHC(O)-L^{2D}-R^{8D}; L^{2D} is independently a bond or an unsubstituted C₂-C₂₂ alkylene; and R^{8D} is independently hydrogen, C₁-C₃ alkyl, or —COOH. In embodiments, R^{1D} is independently —NHC(O)-L^{2D}-R^{8D}. In embodiments, L^{2D} is independently a bond or an unsubstituted C₂-C₂₂ alkylene. In embodiments, L^{2D} is independently a bond. In embodiments, L^{2D} is independently an unsubstituted C₂-C₂₂ alkylene. In embodiments, R^{8D} is independently hydrogen, C₁-C₃ alkyl, or —COOH. In embodiments, R^{8D} is independently hydrogen. In embodiments, R^{8D} is independently C₁-C₃ alkyl. In embodiments, R^{8D} is independently —COOH.

In embodiments, R^{1D} is independently —NHC(O)-L^{2D}-R^{8D}, L^{2D} is independently a bond, and R^{8D} is independently C₁-C₃ alkyl. In embodiments, R^{1D} is independently —NHC(O)—CH₃. In embodiments, R^{1D} is independently —NHC(O)—CH_{2D}H₃. In embodiments, R^{1D} is independently —NHC(O)—CH(CH₃)₂. In embodiments, R^{1D} is independently —NHC(O)—CH_{2D}H_{2D}H₃.

In embodiments, R^{1D} is independently —NHC(O)-L^{2D}-R^{8D}, L^{2D} is independently an unsubstituted C₁₀-C₂₂ alkylene, and R^{8D} is independently —COOH. In embodiments, R^{1D} is independently —NHC(O)-L^{2D}-COOH.

In embodiments, L^{2D} is independently an unsubstituted unbranched C₂-C₂₄ alkylene. In embodiments, L^{2D} is independently an unsubstituted unbranched C₂-C₂₂ alkylene. In embodiments, L^{2D} is independently an unsubstituted unbranched C₅-C₂₂ alkylene. In embodiments, L^{2D} is independently an unsubstituted unbranched C₁₀-C₂₂ alkylene. In embodiments, L^{2D} is independently an unsubstituted unbranched C₁₂-C₂₂ alkylene. In embodiments, L^{2D} is independently an unsubstituted unbranched C₁₀-C₂₀ alkylene. In embodiments, L^{2D} is independently an unsubstituted unbranched C₁₂-C₂₀ alkylene. In embodiments, L^{2D} is independently an unsubstituted unbranched C₁₀-C₁₈ alkylene. In embodiments, L^{2D} is independently an unsubstituted unbranched C₁₂-C₁₈ alkylene. In embodiments, L^{2D} is independently an unsubstituted unbranched C₁₀-C₁₆ alkylene. In embodiments, L^{2D} is independently an unsubstituted unbranched C₁₂-C₁₆ alkylene. In embodiments, L^{2D} is independently an unsubstituted unbranched C₁₄-C₁₆ alkylene. In embodiments, L^{2D} is independently an unsubstituted unbranched C₁₄-C₁₅ alkylene. In embodiments, L^{2D} is independently an unsubstituted unbranched C₁₄ alkylene. In embodiments, L^{2D} is independently an unsubstituted unbranched C₁₅ alkylene. In embodiments, L^{2D} is independently an unsubstituted unbranched C₁₆ alkylene.

In embodiments, L^{2D} is independently an unsubstituted ununbranched saturated C₂-C₂₄ alkylene. In embodiments, L^{2D} is independently an unsubstituted ununbranched saturated C₂-C₂₂ alkylene. In embodiments, L^{2D} is independently an unsubstituted ununbranched saturated C₅-C₂₂ alkylene. In embodiments, L^{2D} is independently an unsubstituted ununbranched saturated C₁₀-C₂₂ alkylene. In embodiments, L^{2D} is independently an unsubstituted ununbranched saturated C₁₂-C₂₂ alkylene. In embodiments, L^{2D} is independently an unsubstituted ununbranched saturated C₁₀-C₂₀ alkylene. In embodiments, L^{2D} is independently an unsubstituted ununbranched saturated C₁₂-C₂₀ alkylene. In embodiments, L^{2D} is independently an unsubstituted ununbranched saturated C₁₀-C₁₈ alkylene. In embodiments, L^{2D} is independently an unsubstituted ununbranched saturated C₁₂-C₁₈ alkylene. In embodiments, L^{2D} is independently an unsubstituted ununbranched saturated C₁₀-C₁₆ alkylene. In embodiments, L^{2D} is independently an unsubstituted ununbranched saturated C₁₂-C₁₆ alkylene. In embodiments, L^{2D} is independently an unsubstituted ununbranched saturated C₁₄-C₁₆ alkylene. In embodiments, L^{2D} is independently an unsubstituted ununbranched saturated C₁₄-C₁₅ alkylene. In embodiments, L^{2D} is independently an unsubstituted ununbranched saturated C₁₄ alkylene. In embodiments, L^{2D} is independently an unsubstituted ununbranched saturated C₁₅ alkylene. In embodiments, L^{2D} is independently an unsubstituted ununbranched saturated C₁₆ alkylene.

In embodiments, L^{2D} is independently an unsubstituted ununbranched unsaturated C₂-C₂₄ alkylene. In embodiments, L^{2D} is independently an unsubstituted ununbranched unsaturated C₂-C₂₂ alkylene. In embodiments, L^{2D} is independently an unsubstituted ununbranched unsaturated C₅-C₂₂ alkylene. In embodiments, L^{2D} is independently an unsubstituted ununbranched unsaturated C₁₀-C₂₂ alkylene. In embodiments, L^{2D} is independently an unsubstituted ununbranched unsaturated C₁₂-C₂₂ alkylene. In embodiments, L^{2D} is independently an unsubstituted ununbranched unsaturated C₁₀-C₂₀ alkylene. In embodiments, L^{2D} is independently an unsubstituted ununbranched unsaturated C₁₂-C₂₀ alkylene. In embodiments, L^{2D} is independently an unsubstituted ununbranched unsaturated C₁₀-C₁₈ alkylene. In embodiments, L^{2D} is independently an unsubstituted ununbranched unsaturated C₁₂-C₁₈ alkylene. In embodiments, L^{2D} is independently an unsubstituted ununbranched unsaturated C₁₀-C₁₆ alkylene. In embodiments, L^{2D} is independently an unsubstituted ununbranched unsaturated C₁₂-C₁₆ alkylene. In embodiments, L^{2D} is independently an unsubstituted ununbranched unsaturated C₁₄-C₁₆ alkylene. In embodiments, L^{2D} is independently an unsubstituted ununbranched unsaturated C₁₄-C₁₅ alkylene. In embodiments, L^{2D} is independently an unsubstituted ununbranched unsaturated C₁₄ alkylene. In embodiments, L^{2D} is independently an unsubstituted ununbranched unsaturated C₁₅ alkylene. In embodiments, L^{2D} is independently an unsubstituted ununbranched unsaturated C₁₆ alkylene.

In embodiments, R^{1E} is independently —NHC(O)-L^{2E}-R^{8E}; L^{2E} is independently a bond or an unsubstituted C₂-C₂₂ alkylene; and R^{8E} is independently hydrogen, C₁-C₃ alkyl, or —COOH. In embodiments, R^{1E} is independently —NHC(O)-L^{2E}-R^{8E}. In embodiments, L^{2E} is independently a bond or an unsubstituted C₂-C₂₂ alkylene. In embodiments, L^{2E} is independently a bond. In embodiments, L^{2E} is independently an unsubstituted C₂-C₂₂ alkylene. In embodiments, R^{8E} is independently hydrogen, C₁-C₃ alkyl, or —COOH. In embodiments, R^{8E} is independently hydrogen. In embodiments, R^{8E} is independently C₁-C₃ alkyl. In embodiments, R^{8E} is independently —COOH.

In embodiments, R^{1E} is independently —NHC(O)-L^{2E}-R^{8E}, L^{2E} is independently a bond, and R^{8E} is independently C₁-C₃ alkyl. In embodiments, R^{1E} is independently —NHC(O)—CH₃. In embodiments, R^{1E} is independently —NHC(O)—CH_{2E}H₃. In embodiments, R^{1E} is independently —NHC(O)—CH(CH₃)₂. In embodiments, R^{1E} is independently —NHC(O)—CH_{2E}H_{2E}H₃.

In embodiments, R^{1E} is independently —NHC(O)-L^{2E}-R^{8E}, L^{2E} is independently an unsubstituted C₁₀-C₂₂ alkylene, and R^{8E} is independently —COOH. In embodiments, R^{1E} is independently —NHC(O)-L^{2E}-COOH.

In embodiments, L^{2E} is independently an unsubstituted unbranched C₂-C₂₄ alkylene. In embodiments, L^{2E} is independently an unsubstituted unbranched C₂-C₂₂ alkylene. In embodiments, L^{2E} is independently an unsubstituted unbranched C₅-C₂₂ alkylene. In embodiments, L^{2E} is independently an unsubstituted unbranched C₁₀-C₂₂ alkylene. In embodiments, L^{2E} is independently an unsubstituted unbranched C₁₂-C₂₂ alkylene. In embodiments, L^{2E} is independently an unsubstituted unbranched C₁₀-C₂₀ alkylene. In embodiments, L^{2E} is independently an unsubstituted unbranched C₁₂-C₂₀ alkylene. In embodiments, L^{2E} is independently an unsubstituted unbranched C₁₀-C₁₈ alkylene. In embodiments, L^{2E} is independently an unsubstituted unbranched C₁₂-C₁₈ alkylene. In embodiments, L^{2E} is independently an unsubstituted unbranched C₁₀-C₁₆ alkylene. In embodiments, L^{2E} is independently an unsubstituted unbranched C₁₂-C₁₆ alkylene. In embodiments, L^{2E} is independently an unsubstituted unbranched C₁₄-C₁₆ alkylene. In embodiments, L^{2E} is independently an unsubstituted unbranched C₁₄-C₁₅ alkylene. In embodiments, L^{2E} is independently an unsubstituted unbranched C₁₄ alkylene. In embodiments, L^{2E} is independently an unsubstituted unbranched C₁₅ alkylene. In embodiments, L^{2E} is independently an unsubstituted unbranched C₁₆ alkylene.

In embodiments, L^{2E} is independently an unsubstituted ununbranched saturated C₂-C₂₄ alkylene. In embodiments, L^{2E} is independently an unsubstituted ununbranched saturated C₂-C₂₂ alkylene. In embodiments, L^{2E} is independently an unsubstituted ununbranched saturated C₅-C₂₂ alkylene. In embodiments, L^{2E} is independently an unsubstituted ununbranched saturated C₁₀-C₂₂ alkylene. In embodiments, L^{2E} is independently an unsubstituted ununbranched saturated C₁₂-C₂₂ alkylene. In embodiments, L^{2E} is independently an unsubstituted ununbranched saturated C₁₀-C₂₀ alkylene. In embodiments, L^{2E} is independently an unsubstituted ununbranched saturated C₁₂-C₂₀ alkylene. In embodiments, L^{2E} is independently an unsubstituted ununbranched saturated C₁₀-C₁₈ alkylene. In embodiments, L^{2E} is independently an unsubstituted ununbranched saturated C₁₂-C₁₈ alkylene. In embodiments, L^{2E} is independently an unsubstituted ununbranched saturated C₁₀-C₁₆ alkylene. In embodiments, L^{2E} is independently an unsubstituted ununbranched saturated C₁₂-C₁₆ alkylene. In embodiments, L^{2E} is independently an unsubstituted ununbranched saturated C₁₄-C₁₆ alkylene. In embodiments, L^{2E} is independently an unsubstituted ununbranched saturated C₁₄-C₁₅ alkylene. In embodiments, L^{2E} is independently an unsubstituted ununbranched saturated C₁₄ alkylene. In embodiments, L^{2E} is independently an unsubstituted ununbranched saturated C₁₅ alkylene. In embodiments, L^{2E} is independently an unsubstituted ununbranched saturated C₁₆ alkylene.

In embodiments, L^{2E} is independently an unsubstituted ununbranched unsaturated C₂-C₂₄ alkylene. In embodiments, L^{2E} is independently an unsubstituted ununbranched unsaturated C₂-C₂₂ alkylene. In embodiments, L^{2E} is independently an unsubstituted ununbranched unsaturated C₅-C₂₂ alkylene. In embodiments, L^{2E} is independently an unsubstituted ununbranched unsaturated C₁₀-C₂₂ alkylene. In embodiments, L^{2E} is independently an unsubstituted ununbranched unsaturated C₁₂-C₂₂ alkylene. In embodiments, L^{2E} is independently an unsubstituted ununbranched unsaturated C₁₀-C₂₀ alkylene. In embodiments, L^{2E} is independently an unsubstituted ununbranched unsaturated C₁₂-C₂₀ alkylene. In embodiments, L^{2E} is independently an unsubstituted ununbranched unsaturated C₁₀-C₁₈ alkylene. In embodiments, L^{2E} is independently an unsubstituted ununbranched unsaturated C₁₂-C₁₈ alkylene. In embodiments, L^{2E} is independently an unsubstituted ununbranched unsaturated C₁₀-C₁₆ alkylene. In embodiments, L^{2E} is independently an unsubstituted ununbranched unsaturated C₁₂-C₁₆ alkylene. In embodiments, L^{2E} is independently an unsubstituted ununbranched unsaturated C₁₄-C₁₆ alkylene. In embodiments, L^{2E} is independently an unsubstituted ununbranched unsaturated C₁₄-C₁₅ alkylene. In embodiments, L^{2E} is independently an unsubstituted ununbranched unsaturated C₁₄ alkylene. In embodiments, L^{2E} is independently an unsubstituted ununbranched unsaturated C₁₅ alkylene. In embodiments, L^{2E} is independently an unsubstituted ununbranched unsaturated C₁₆ alkylene.

In embodiments, L^{1C} is independently R^{1C}-substituted or unsubstituted 5 to 8 membered heteroalkylene, L^{1D} is independently a bond, or R^{1D}-substituted or unsubstituted 5 to 8 membered heteroalkylene, and L^{1E} is independently R^{1E}-substituted or unsubstituted 5 to 8 membered heteroalkylene or —NHC(O)—. In embodiments, each R^{1C}, R^{1D}, or R^{1E} is independently oxo, or —COOH.

In embodiments, L^{1C} is independently unsubstituted 5 to 8 membered heteroalkylene. In embodiments, L^{1D} is independently oxo-substituted or unsubstituted 5 to 8 membered heteroalkylene. In embodiments, L^{1E} is independently R^{1E}-substituted or unsubstituted 5 to 8 membered heteroalkylene wherein R^{1E} is independently oxo, or —COOH. In embodiments, L^{1C} is independently unsubstituted 5 to 8 membered heteroalkylene. In embodiments, L^{1D} is independently a bond. In embodiments, L^{1E} is independently —NHC(O)—. In embodiments, -L^{1C}-L^{1D}-L^{1E}- may form

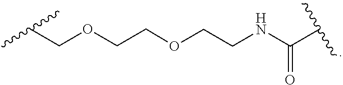 [SMILES: *COCCOCCNC(*)=O]

In embodiments, -L^{1C}-L^{1D}-L^{1E}- may form

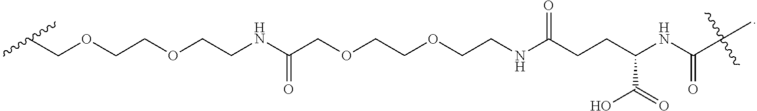 [SMILES: *COCCOCCNC(=O)COCCOCCNC(=O)CC[C@H](NC(*)=O)C(=O)O]

In embodiments, L^{1C} is independently R^{1C}-substituted C₂-C₅ alkyl; L^{1D} is independently an unsubstituted phenylene, or an unsubstituted biphenylene; L^{1E} is independently R^{1E}-substituted or unsubstituted 5 to 8 membered heteroalkylene or —NHC(O)—; R^{1C} is independently —NHC(O)-L^{2C}-R^{8C}; L^{2C} is independently a bond, or an unsubstituted C₁₀-C₂₂ alkylene; R^{8C} is independently an unsubstituted C₁-C₃ alkyl, or —COOH; and R^{1E} is oxo.

In embodiments, L^{1C} is independently R^{1C}-substituted ethylene. In embodiments, L^{1D} is independently an unsubstituted biphenylene. In embodiments, L^{1E} is independently —NHC(O)—. In embodiments, R^{1C} is independently —NHC(O)-L^{2C}-R^{8C}, and L^{2C} is independently a bond or R^{8C} is independently an unsubstituted C₁-C₃ alkyl. In embodiments, R^{1C} is independently —NHC(O)—CH₃. In embodiments, R^{1C} is independently —NHC(O)—CH₂CH₃. In embodiments, R^{1C} is independently —NHC(O)—CH(CH₃)₂. In embodiments, R^{1C} is independently —NHC(O)—CH₂CH₂CH₃. In embodiments, R^{1C} is independently —NHC(O)-L^{2C}-COOH. In embodiments, -L^{1C}-L^{1D}-L^{1E}- may form

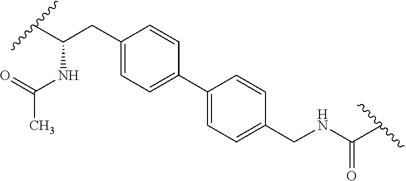 [SMILES: *C(=O)NCc1ccc(-c2ccc(C[C@@H](*)NC(C)=O)cc2)cc1]

In embodiments, -L^{1C}-L^{1D}-L^{1E}- may form

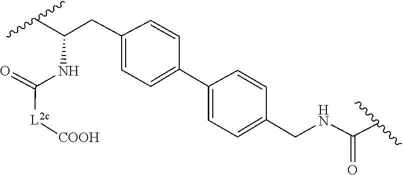

In embodiments, -L^{1C}-L^{1D}-L^{1E}- may form

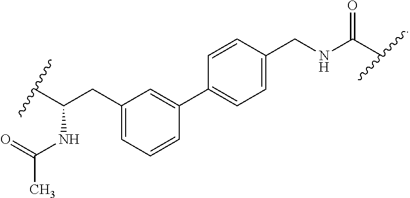 [SMILES: *C(=O)NCc1ccc(-c2cccc(C[C@@H](*)NC(C)=O)c2)cc1]

In embodiments, -L^{1C}-L^{1D}-L^{1E}- may form

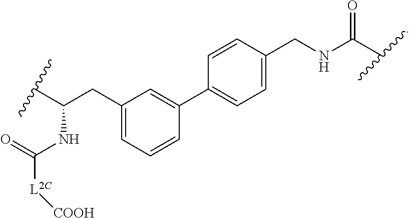

In embodiments, L^{1C} is independently R^{1C}-substituted ethylene. In embodiments, L^{1D} is independently an unsubstituted phenylene. In embodiments, L^{1E} is independently R^{1E}-substituted or unsubstituted 5 to 8 membered heteroalkylene. In embodiments, L^{1E} is independently oxo-substituted or unsubstituted 5 to 8 membered heteroalkylene. In embodiments, R^{1C} is independently —NHC(O)-L^{2C}-R^{8C}, and L^{2C} is independently a bond or R^{8C} is independently an unsubstituted C₁-C₃ alkyl. In embodiments, R^{1C} is independently —NHC(O)—CH₃. In embodiments, R^{1C} is independently —NHC(O)—CH₂CH₃. In embodiments, R^{1C} is independently —NHC(O)—CH(CH₃)₂. In embodiments, R^{1C} is independently —NHC(O)—CH₂CH₂CH₃. In embodiments, R^{1C} is independently —NHC(O)-L^{2C}-COOH. In embodiments, -L^{1C}-L^{1D}-L^{1E}- may form

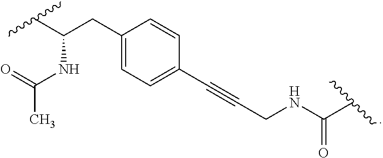 [SMILES: *C(=O)NCC#Cc1ccc(C[C@@H](*)NC(C)=O)cc1]

In embodiments, -L^{1C}-L^{1D}-L^{1E}- may form

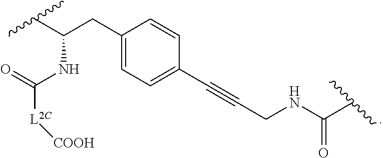

In embodiments, -L^{1C}-L^{1D}-L^{1E}- may form

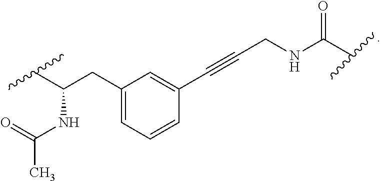 [SMILES: *C(=O)NCC#Cc1cccc(C[C@@H](*)NC(C)=O)c1]

In embodiments, -L^{1C}-L^{1D}-L^{1E}- may form

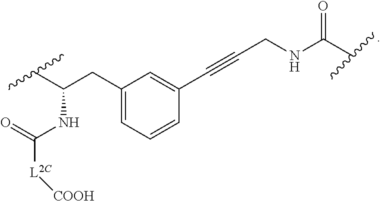

In embodiments, L^{1C} is independently R^{1C}-substituted or unsubstituted ethylene or n-pentylene, L^{1D} is independently a bond, L^{1E} is independently —NHC(O)—, R^{1C} is independently —NHC(O)-L^{2C}-R^{8C}, L^{2C} is independently a bond or an unsubstituted C₁₀-C₂₂ alkylene, and R^{8C} is independently an unsubstituted C₁-C₃ alkyl, or —COOH.

In embodiments, L^{1C} is independently R^{1C}-substituted n-pentylene. In embodiments, L^{1C} is independently unsubstituted n-pentylene. In embodiments, L^{1D} is independently a bond. In embodiments, L^{1E} is independently —NHC(O)—. In embodiments, R^{1C} is independently —NHC(O)-L^{2C}-R^{8C}, and L^{2C} is independently a bond and R^{8C} is independently an unsubstituted C₁-C₃ alkyl. In embodiments, R^{1C} is independently —NHC(O)—CH₃. In embodiments, R^{1C} is independently —NHC(O)—CH₂CH₃. In embodiments, R^{1C} is independently —NHC(O)—CH(CH₃)₂. In embodiments, R^{1C} is independently —NHC(O)—CH₂CH₂CH₃. In embodiments, R^{1C} is independently —NHC(O)-L^{2C}-COOH. In embodiments, -L^{1C}-L^{1D}-L^{1E}- may form

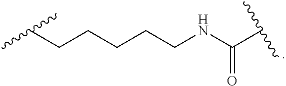 [SMILES: *CCCCCNC(*)=O]

In embodiments, -L^{1C}-L^{1D}-L^{1E}- may form

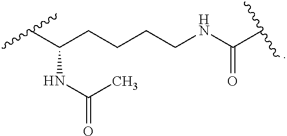 [SMILES: *C(=O)NCCCC[C@@H](*)NC(C)=O]

In embodiments, -L^{1C}-L^{1D}-L^{1E}- may form

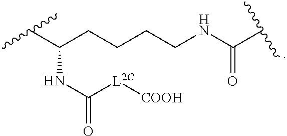

In embodiments, L^{1C} is independently R^{1C}-substituted ethylene. In embodiments, L^{1D} is independently a bond. In embodiments, L^{1E} is independently —NHC(O)—. In embodiments, R^{1C} is independently —NHC(O)-L^{2C}-R^{8C}, and L^{2C} is independently a bond and R^{8C} is independently an unsubstituted C₁-C₃ alkyl. In embodiments, R^{1C} is independently —NHC(O)—CH₃. In embodiments, R^{1C} is independently —NHC(O)—CH₂CH₃. In embodiments, R^{1C} is independently —NHC(O)—CH(CH₃)₂. In embodiments, R^{1C} is independently —NHC(O)—CH₂CH₂CH₃. In embodiments, R^{1C} is independently —NHC(O)-L^{2C}-R^{8C}, L^{2C} is independently an unsubstituted C₁₀-C₂₂ alkylene, and R^{8C} is independently —COOH. In embodiments, R^{1C} is independently —NHC(O)-L^{2C}-COOH. In embodiments, -L^{1C}-L^{1D}-L^{1E}- may form

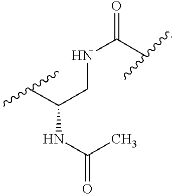 [SMILES: *C(=O)NC[C@@H](*)NC(C)=O]

In embodiments, -L^{1C}-L^{1D}-L^{1E}- may form

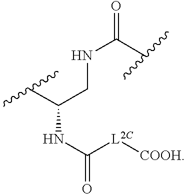

In embodiments, L^{1C} is independently R^{1C}-substituted or unsubstituted n-pentylene, L^{1D} is independently oxo-substituted or unsubstituted 5 to 8 membered heteroalkylene, L^{1E} is independently —NHC(O)—, R^{1C} is independently —NHC(O)-L^{2C}-R^{8C}, L^{2C} is independently a bond or an unsubstituted C₁₀-C₂₂ alkylene, and R^{8C} is independently an unsubstituted C₁-C₃ alkyl, or —COOH.

In embodiments, L^{1C} is independently R^{1C}-substituted n-pentylene. In embodiments, L^{1D} is independently oxo-substituted or unsubstituted 5 to 8 membered heteroalkylene. In embodiments, L^{1E} is independently —NHC(O)—. In embodiments, R^{1C} is independently —NHC(O)-L^{2C}-R^{8C}, L^{2C} is independently a bond, and R^{8C} is independently an unsubstituted C₁-C₃ alkyl. In embodiments, R^{1C} is independently —NHC(O)—CH₃. In embodiments, R^{1C} is independently —NHC(O)—CH₂CH₃. In embodiments, R^{1C} is independently —NHC(O)—CH(CH₃)₂. In embodiments, R^{1C} is independently —NHC(O)—CH₂CH₂CH₃. In embodiments, R^{1C} is independently —NHC(O)-L^{2C}-R^{8C}, L^{2C} is independently an unsubstituted C₁₀-C₂₂ alkylene, and R^{8C} is independently an unsubstituted C₁-C₃ alkyl. In embodiments, R^{1C} is independently —NHC(O)-L^{2C}-R^{8C}, L^{2C} is independently an unsubstituted C₁₀-C₂₂ alkylene, and R^{8C} is independently an unsubstituted methyl. In embodiments, R^{1C} is independently —NHC(O)-L^{2C}-R^{8C}, L^{2C} is independently an unsubstituted C₁₀-C₂₂ alkylene, and R^{8C} is independently an unsubstituted ethyl. In embodiments, R^{1C} is independently —NHC(O)-L^{2C}-R^{8C}, L^{2C} is independently an unsubstituted C₁₀-C₂₂ alkylene, and R^{8C} is independently —COOH. In embodiments, R^{1C} is independently —NHC(O)-L^{2C}-COOH. In embodiments, R^{1C} is independently —NHC(O)-L^{2C}-R^{8C}, L^{2C} is independently an unsubstituted C₁₀-C₂₂ alkylene, and R^{8C} is independently —COOH. In embodiments, -L^{1C}-L^{1D}-L^{1E}- may form

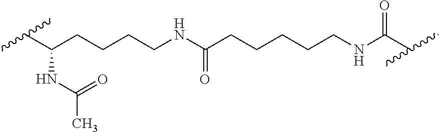 [SMILES: *C(=O)NCCCCCC(=O)NCCCC[C@@H](*)NC(C)=O]

In embodiments, -L^{1C}-L^{1D}-L^{1E}- may form

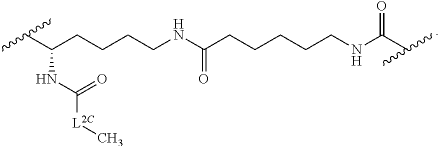

In embodiments, -L^{1C}-L^{1D}-L^{1E}- may form

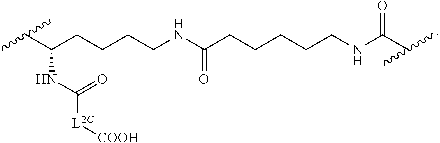

In embodiments, -L^{1C}-L^{1D}-L^{1E}- may form

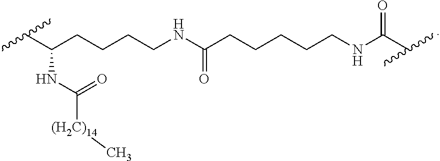

In embodiments, -L^{1C}-L^{1D}-L^{1E}- may form

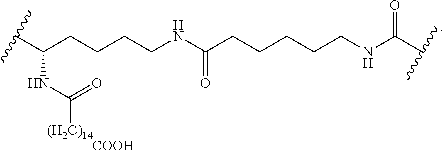

In embodiments, L^{1C} is independently R^{1C}-substituted methylene, L^{1D} is independently a bond, L^{1E} is independently —NHC(O)—, R^{1C} is independently -L^{8C}-L^{2C}-R^{8C}, L^{8C} is independently an unsubstituted C₁-C₆ alkylene or oxo-substituted 2 to 12 membered heteroalkylene, L^{2C} is independently a bond, and R^{8C} is independently an unsubstituted C₁-C₆ alkyl or oxo-substituted 2 to 12 membered heteroalkyl.

In embodiments, L^{1C} is independently R^{1C}-substituted methylene. In embodiments, L^{1D} is independently a bond. In embodiments, L^{1E} is independently —NHC(O)—. In embodiments, R^{1C} is independently -L^{8C}-L^{2C}-R^{1C}. In embodiments, L^{8C} is independently an unsubstituted C₁-C₆ alkylene or oxo-substituted 2 to 12 membered heteroalkylene. In embodiments, L^{8C} is independently an unsubstituted C₁-C₆ alkylene. In embodiments, L^{8C} is independently oxo-substituted 2 to 12 membered heteroalkylene. In embodiments, R^{8C} is independently an unsubstituted C₁-C₆ alkyl. In embodiments, R^{8C} is independently oxo-substituted 2 to 12 membered heteroalkyl. In embodiments, L^{8C} is independently unsubstituted C₁-C₆ alkylene, L^{2C} is a bond, and R^{8C} is independently oxo-substituted 2 to 12 membered heteroalkyl. In embodiments, L^{8C} is independently unsubstituted C₄ alkylene, L^{2C} is a bond, and R^{8C} is independently oxo-substituted 2-5 membered heteroalkyl. In embodiments, L^{8C} is independently unsubstituted C₄ alkylene, L^{2C} is a bond, and R^{8C} is independently oxo-substituted and C₁-C₂₀ alkyl substituted 2 to 12 membered heteroalkyl. In embodiments, L^{8C} is independently unsubstituted C₁-C₆ alkylene, L^{2C} is a bond, and R^{8C} is independently oxo- and C₁-C₁₅ alkyl-substituted 2 to 12 membered heteroalkyl. In embodiments, L^{8C} is independently unsubstituted C₄ alkylene, L^{2C} is a bond, and R^{8C} is independently oxo- and C₁₄-C₁₅ alkyl-substituted 2 to 12 membered heteroalkyl. In embodiments, L^{8C} is independently oxo-substituted 2 to 12 membered heteroalkylene, L^{2C} is a bond, R^{8C} is independently oxo-substituted 2 to 12 membered heteroalkyl. In embodiments, L^{8C} is independently oxo-substituted 2 to 12 membered heteroalkylene, L^{2C} is a bond, R^{8C} is independently oxo- and C₁-C₁₅ alkyl-substituted 2 to 12 membered heteroalkyl. In embodiments, L^{8C} is independently oxo-substituted 2 to 12 membered heteroalkylene, L^{2C} is a bond, R^{8C} is independently oxo- and C₁₄-C₁₅ alkyl-substituted 2 to 12 membered heteroalkyl. In embodiments, -L^{1C}-L^{1D}-L^{1E}- may form

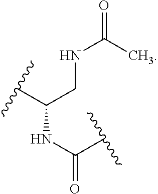 [SMILES: *C(=O)N[C@H](*)CNC(C)=O]

In embodiments, -L^{1C}-L^{1D}-L^{1E}- may form

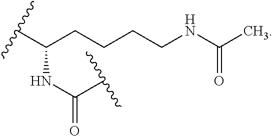 [SMILES: *C(=O)N[C@H](*)CCCCNC(C)=O]

In embodiments, -L^{1C}-L^{1D}-L^{1E}- may form

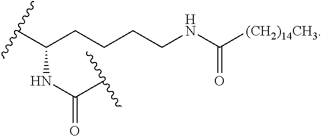

In embodiments, -L^{1C}-L^{1D}-L^{1E}- may form

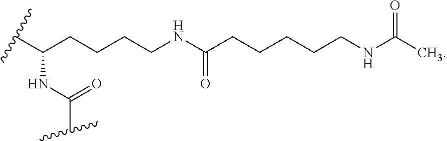 [SMILES: *C(=O)N[C@H](*)CCCCNC(=O)CCCCCNC(C)=O]

In embodiments, L¹ is

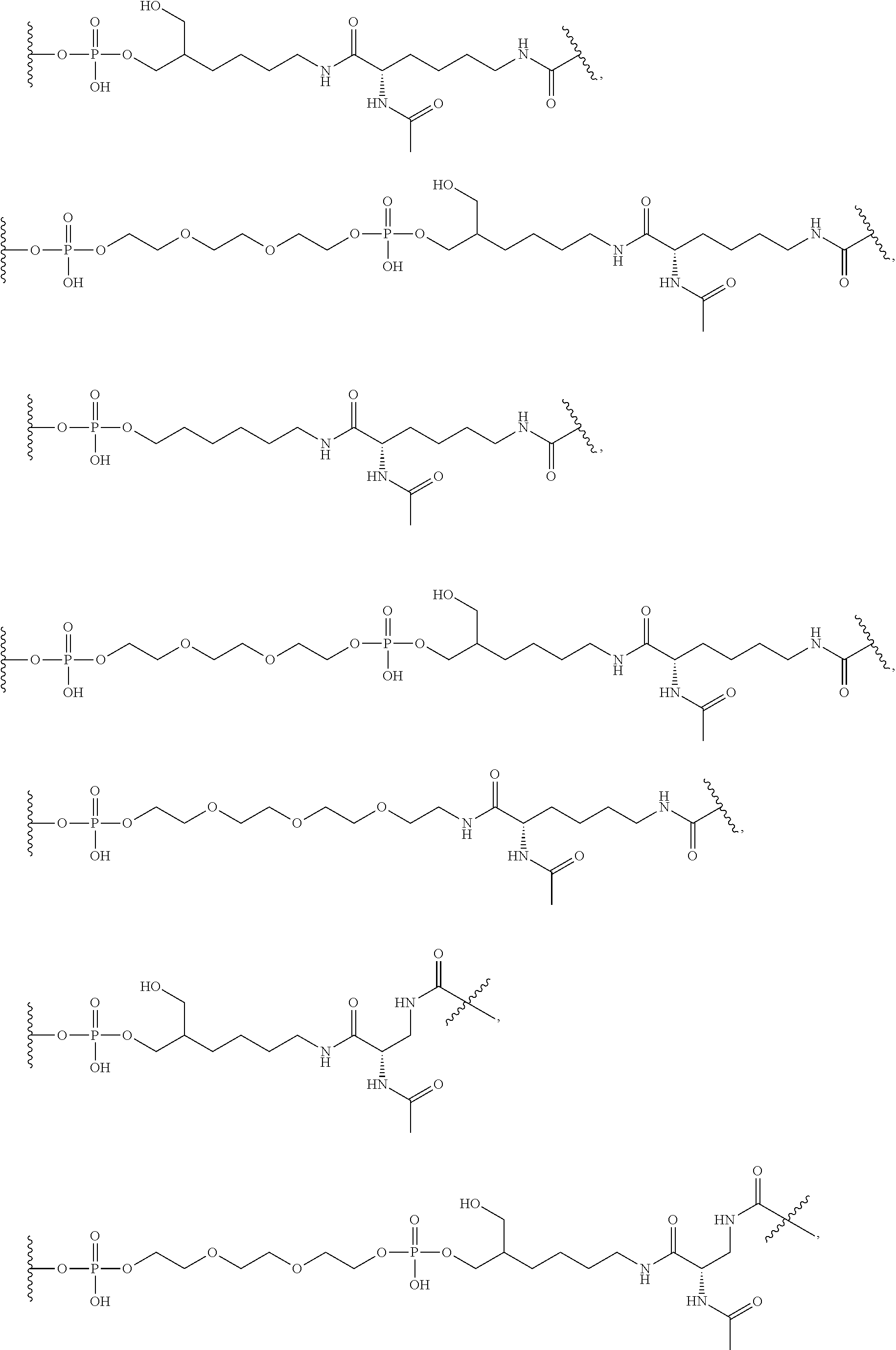

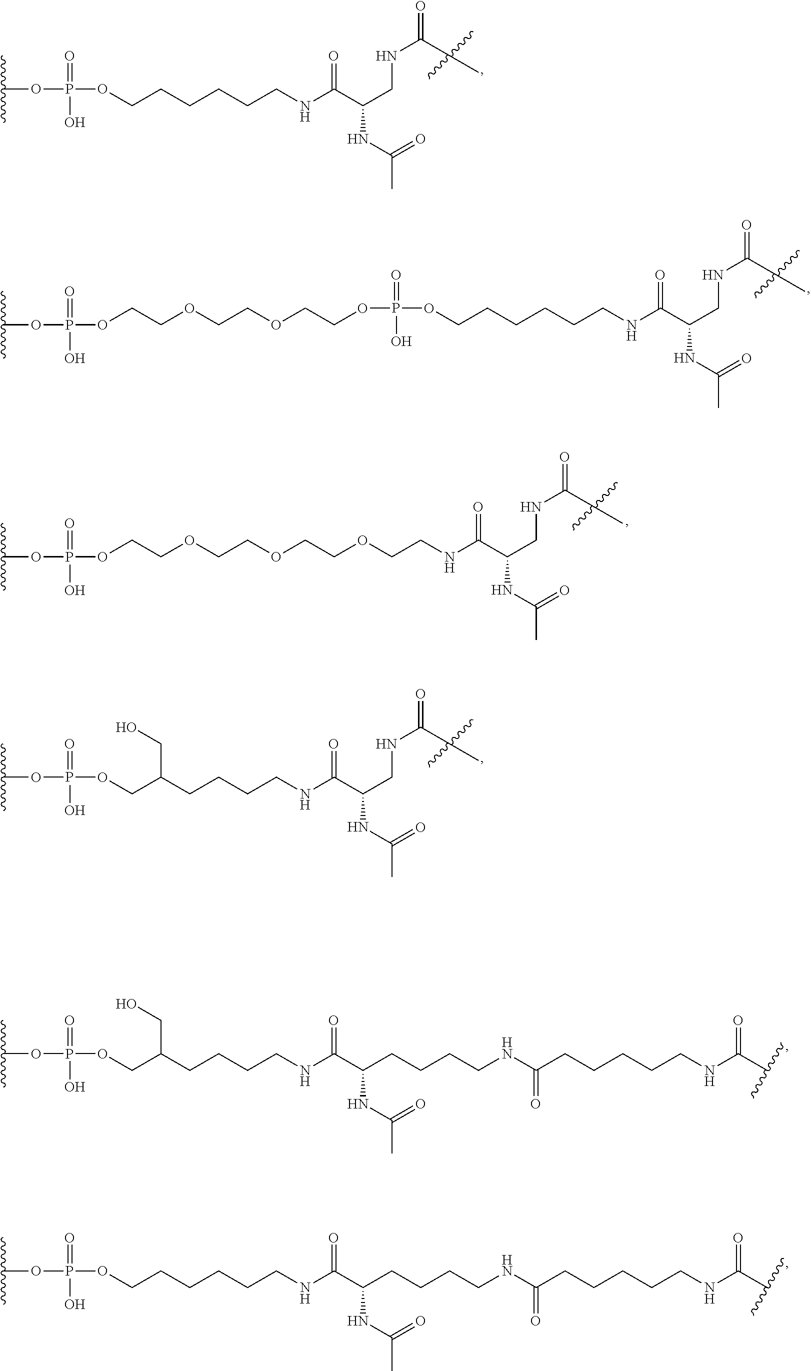 [SMILES: *OP(=O)(O)OCCCCCCNC(=O)[C@H](CNC(*)=O)NC(C)=O ; *OP(=O)(O)OCCOCCOCCOP(=O)(O)OCCCCCCNC(=O)[C@H](CNC(*)=O)NC(C)=O ; *OP(=O)(O)OCCOCCOCCOCCNC(=O)[C@H](CNC(*)=O)NC(C)=O ; *OP(=O)(O)OCC(CO)CCCCNC(=O)[C@H](CNC(*)=O)NC(C)=O ; *OP(=O)(O)OCC(CO)CCCCNC(=O)[C@H](CCCCNC(=O)CCCCCNC(*)=O)NC(C)=O ; *OP(=O)(O)OCCCCCCNC(=O)[C@H](CCCCNC(=O)CCCCCNC(*)=O)NC(C)=O]

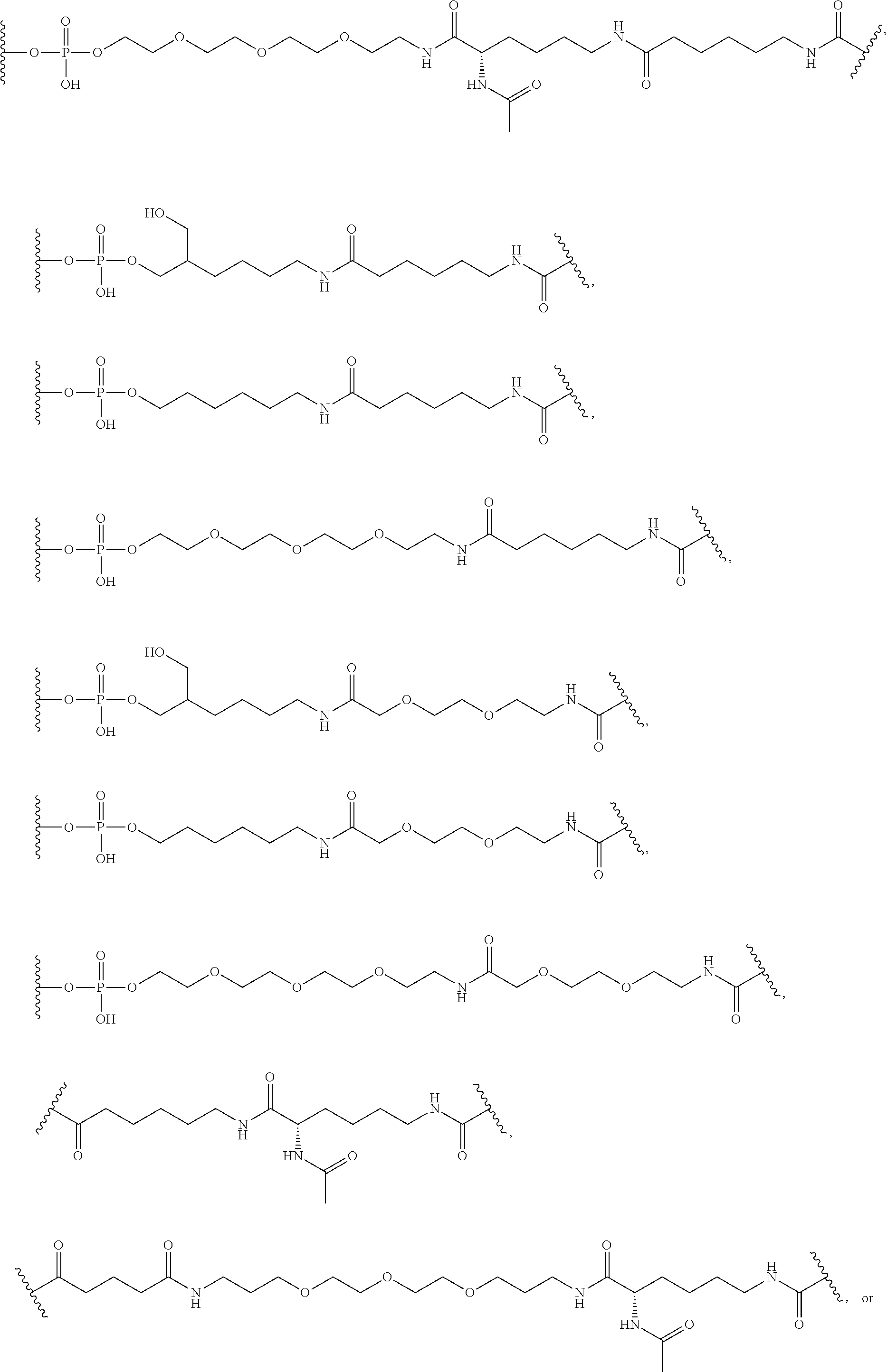

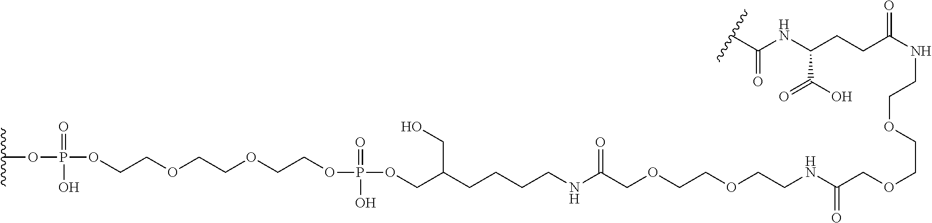 [SMILES: *OP(=O)(O)OCCOCCOCCOP(=O)(O)OCC(CO)CCCCNC(=O)COCCOCCNC(=O)COCCOCCNC(=O)CC[C@@H](NC(*)=O)C(=O)O]

In embodiments, L¹ is independently

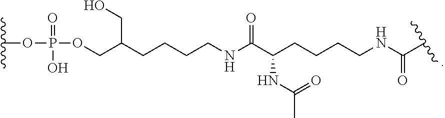 [SMILES: *OP(=O)(O)OCC(CO)CCCCNC(=O)[C@H](CCCCNC(*)=O)NC(C)=O]

In embodiments, L¹ is independently

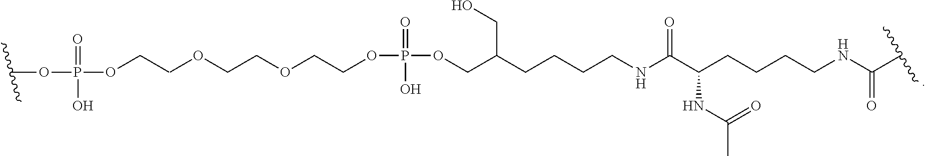

In embodiments, L¹ is independently

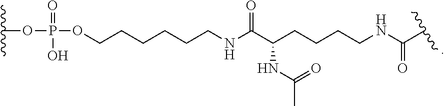 [SMILES: *OP(=O)(O)OCCCCCCNC(=O)[C@H](CCCCNC(*)=O)NC(C)=O]

In embodiments, L¹ is independently

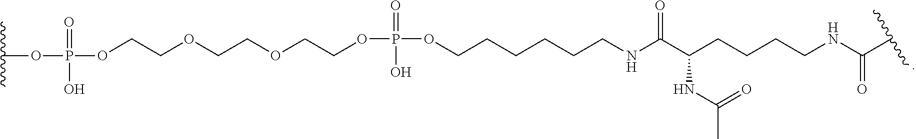 [SMILES: *OP(=O)(O)OCCOCCOCCOP(=O)(O)OCCCCCCNC(=O)[C@H](CCCCNC(*)=O)NC(C)=O]

In embodiments, L¹ is independently

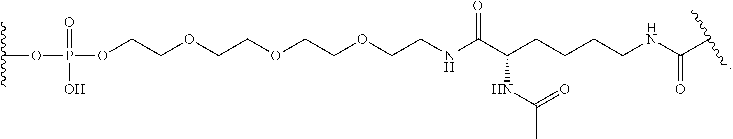 [SMILES: *OP(=O)(O)OCCOCCOCCOCCNC(=O)[C@H](CCCCNC(*)=O)NC(C)=O]

In embodiments, L¹ is independently

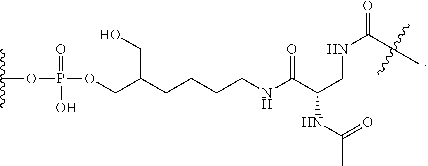 [SMILES: *OP(=O)(O)OCC(CO)CCCCNC(=O)[C@H](CNC(*)=O)NC(C)=O]

In embodiments, L¹ is independently

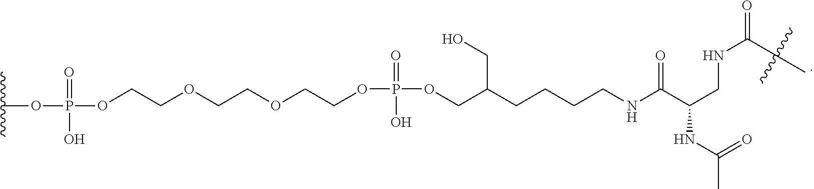 [SMILES: *OP(=O)(O)OCCOCCOCCOP(=O)(O)OCC(CO)CCCCNC(=O)[C@H](CNC(*)=O)NC(C)=O]

In embodiments, L¹ is independently

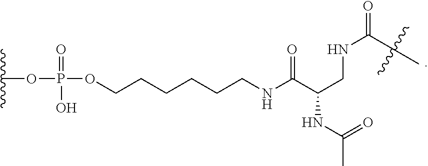 [SMILES: *OP(=O)(O)OCCCCCCNC(=O)[C@H](CNC(*)=O)NC(C)=O]

In embodiments, L¹ is independently

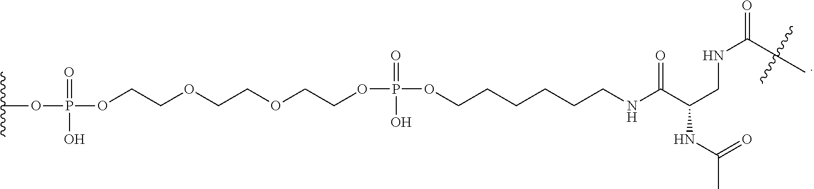 [SMILES: *OP(=O)(O)OCCOCCOCCOP(=O)(O)OCCCCCCNC(=O)[C@H](CNC(*)=O)NC(C)=O]

In embodiments, L¹ is independently

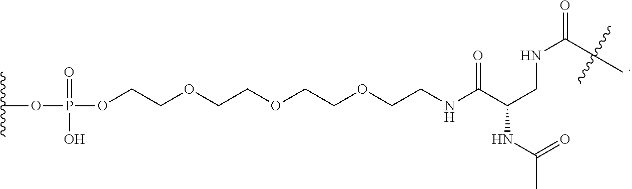 [SMILES: *OP(=O)(O)OCCOCCOCCOCCNC(=O)[C@H](CNC(*)=O)NC(C)=O]

In embodiments, L¹ is independently

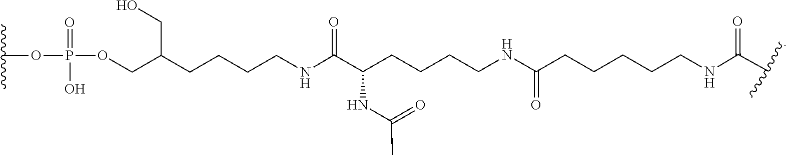 [SMILES: *OP(=O)(O)OCC(CO)CCCCNC(=O)[C@H](CCCCNC(=O)CCCCCNC(*)=O)NC(C)=O]

In embodiments, L¹ is independently

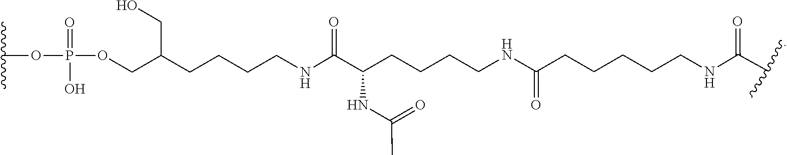 [SMILES: *OP(=O)(O)OCC(CO)CCCCNC(=O)[C@H](CCCCNC(=O)CCCCCNC(*)=O)NC(C)=O]

In embodiments, L¹ is independently

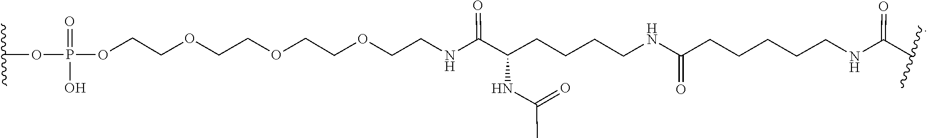 [SMILES: *OP(=O)(O)OCCOCCOCCOCCNC(=O)[C@H](CCCCNC(=O)CCCCCNC(*)=O)NC(C)=O]

In embodiments, L¹ is independently

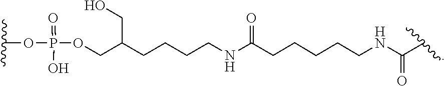 [SMILES: *OP(=O)(O)OCC(CO)CCCCNC(=O)CCCCCNC(*)=O]

In embodiments, L¹ is independently

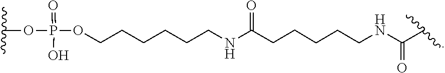 [SMILES: *OP(=O)(O)OCCCCCCNC(=O)CCCCCNC(*)=O]

In embodiments, L¹ is independently

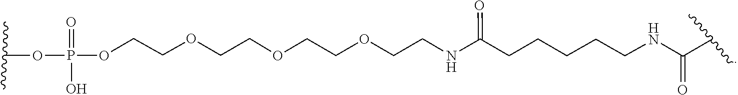 [SMILES: *OP(=O)(O)OCCOCCOCCOCCNC(=O)CCCCCNC(*)=O]

In embodiments, L¹ is independently

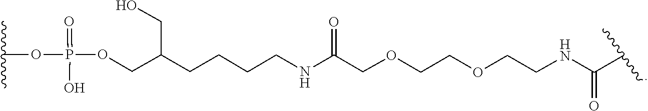 [SMILES: *OP(=O)(O)OCC(CO)CCCCNC(=O)COCCOCCNC(*)=O]

In embodiments, L¹ is independently

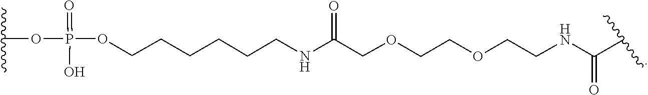 [SMILES: *OP(=O)(O)OCCCCCCNC(=O)COCCOCCNC(*)=O]

In embodiments, L¹ is independently

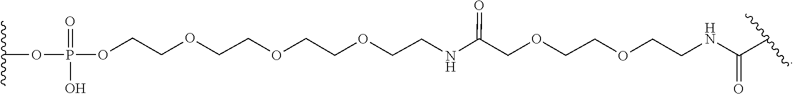 [SMILES: *OP(=O)(O)OCCOCCOCCOCCNC(=O)COCCOCCNC(*)=O]

In embodiments, L¹ is independently

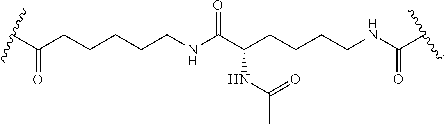 [SMILES: *C(=O)CCCCCNC(=O)[C@H](CCCCNC(*)=O)NC(C)=O]

In embodiments, L¹ is independently

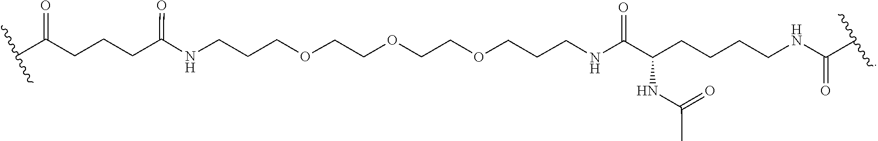 [SMILES: *C(=O)CCCC(=O)NCCCOCCOCCOCCCNC(=O)[C@H](CCCCNC(*)=O)NC(C)=O]

In embodiments, L¹ is independently.

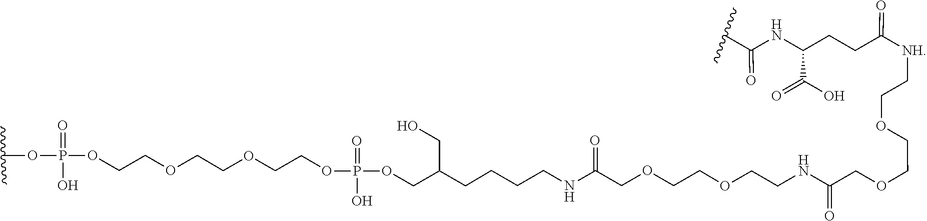 [SMILES: *OP(=O)(O)OCCOCCOCCOP(=O)(O)OCC(CO)CCCCNC(=O)COCCOCCNC(=O)COCCOCCNC(=O)CC[C@@H](NC(*)=O)C(=O)O]

In embodiments, L¹ is

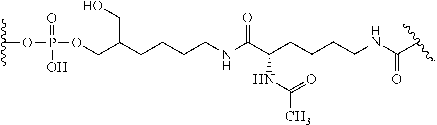 [SMILES: *OP(=O)(O)OCC(CO)CCCCNC(=O)[C@H](CCCCNC(*)=O)NC(C)=O]

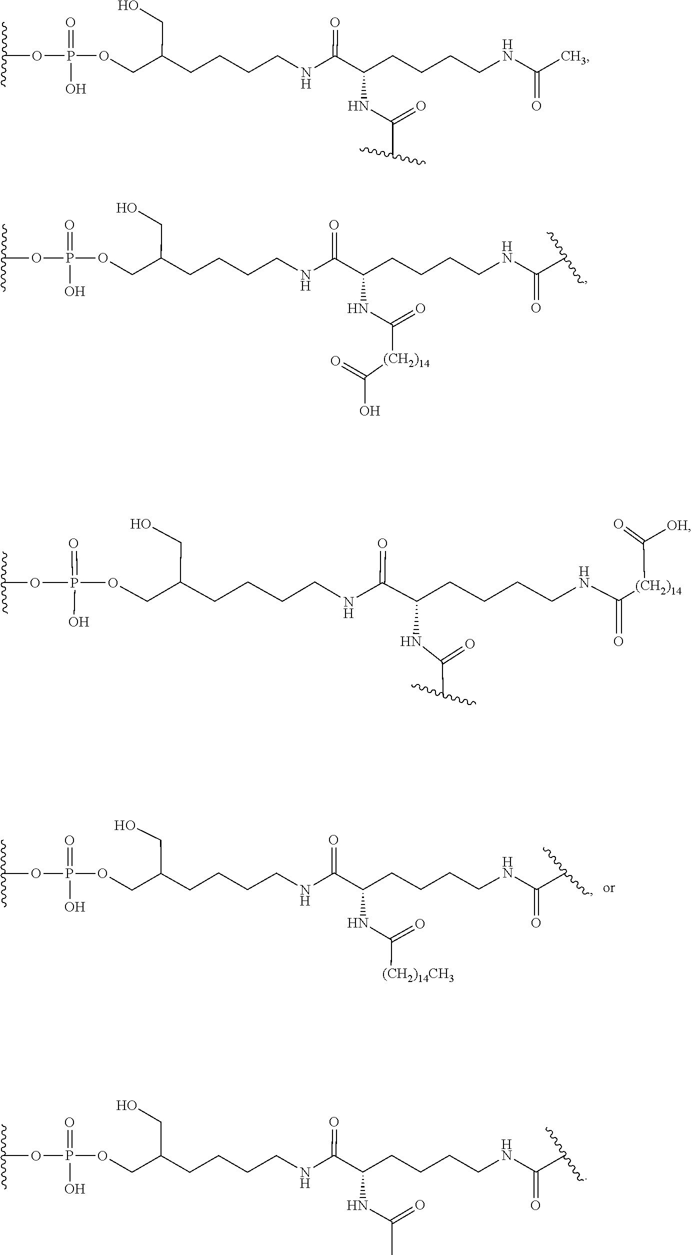

In embodiments, L¹ is

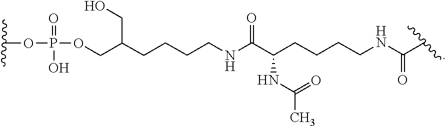 [SMILES: *OP(=O)(O)OCC(CO)CCCCNC(=O)[C@H](CCCCNC(*)=O)NC(C)=O]

In embodiments, L¹ is

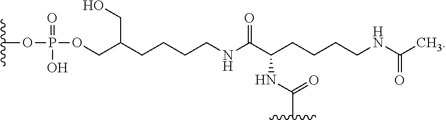 [SMILES: *OP(=O)(O)OCC(CO)CCCCNC(=O)[C@H](CCCCNC(C)=O)NC(*)=O]

In embodiments, L¹ is

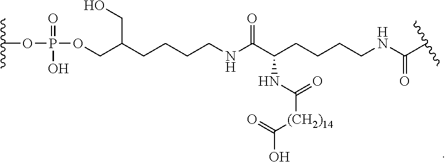

In embodiments, L¹ is

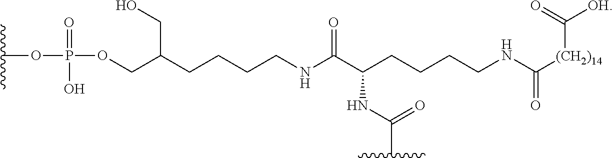

In embodiments, L¹ is

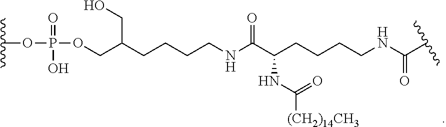

In embodiments, L¹ is

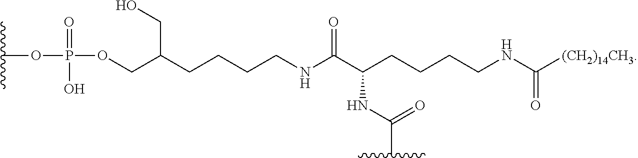

In embodiments, the HLEM is

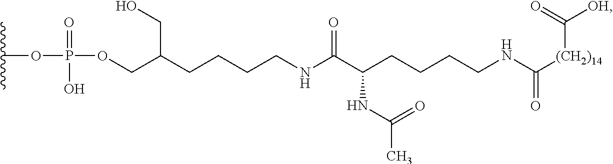

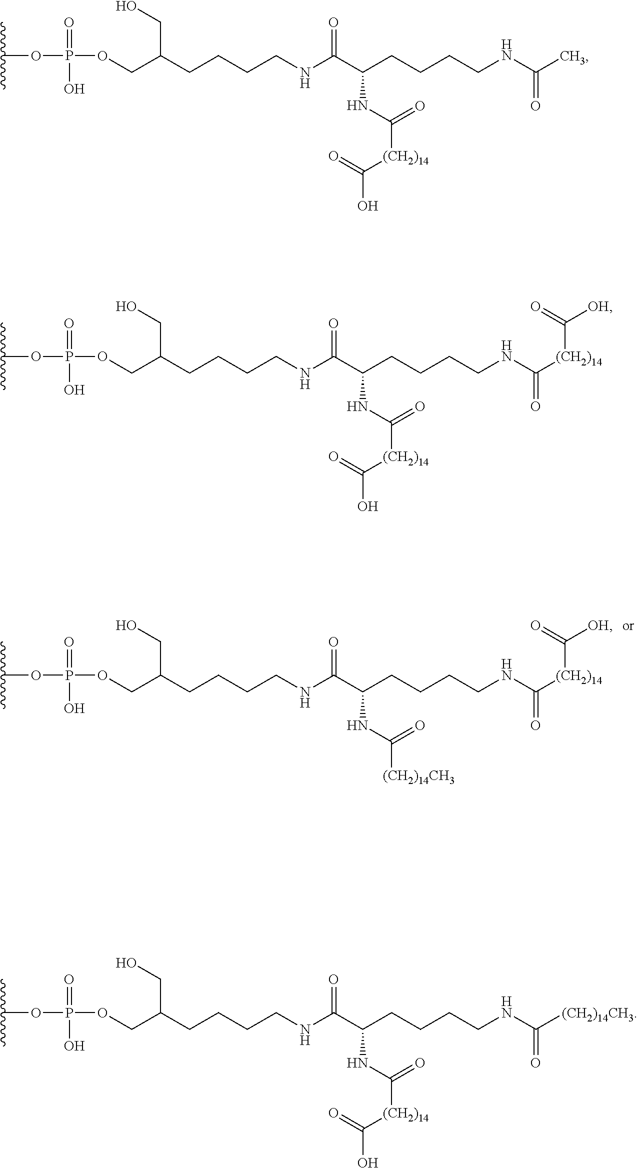

In embodiments, the HLEM is

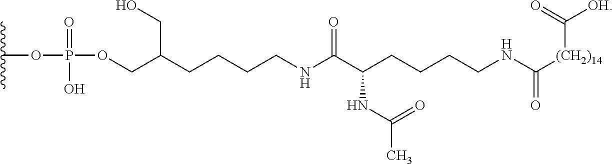

In embodiments, the HLEM is

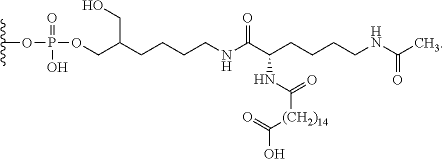

In embodiments, the HLEM is

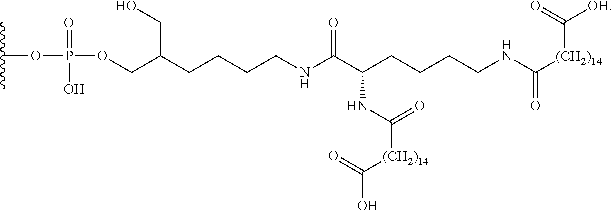

In embodiments, the HLEM is

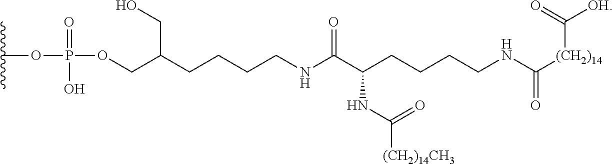

In embodiments, the HLEM is

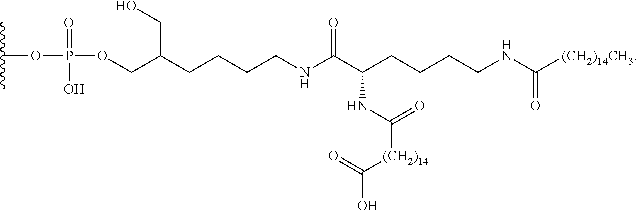

In embodiments, the compound includes from one to five optionally different half-life extension motifs. In embodiments, the compound includes from one to four optionally different half-life extension motifs. In embodiments, the compound includes from one to three optionally different half-life extension motifs. In embodiments, the compound includes from one to two optionally different half-life extension motifs. In embodiments, the compound includes from two to five different half-life extension motifs. In embodiments, the compound includes from two to four different half-life extension motifs. In embodiments, the compound includes from two to three different half-life extension motifs. In embodiments, the compound includes two different half-life extension motifs. In embodiments, compound includes only one half-life extension motif.

In embodiments, the uptake motif independently has the structure:

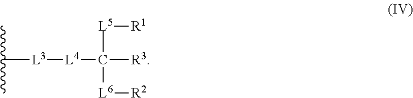

L³ and L⁴ are independently a bond, —N(R²³)—, —O—, —S—, —C(O)—, —N(R²³)C(O)—, —C(O)N(R²⁴)—, —N(R²³)C(O)N(R²⁴)—, —C(O)O—, —OC(O)—, —N(R²³)C(O)O—, —OC(O)N(R²⁴)—, —OPO₂—O—, —O—P(O)(S)—O—, —O—P(O)(R²⁵)—O—, —O—P(S)(R²⁵)—O—, —O—P(O)(NR²³R²⁴)—N—, —O—P(S)(NR²³R²⁴)—N—, —O—P(O)(NR²³R²⁴)—O—, —O—P(S)(NR²³R²⁴)—O—, —P(O)(NR²³R²⁴)—N—, —P(S)(NR²³R²⁴)—N—, —P(O)(NR²³R²⁴)—O—, —P(S)(NR²³R²⁴)—O—, —S—S—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene. Each R²³, R²⁴ and R²⁵ is independently hydrogen or unsubstituted C₁-C₁₀ alkyl.

L⁵ is -L^{5A}-L^{5B}-L^{5C}-L^{5D}-L^{5E}- and L⁶ is -L^{6A}-L^{6B}-L^{6C}-L^{6D}-L^{6E}-L^{5A}, L^{5B}, L^{5C}, L^{5D}, L^{5E}, L^{6A}, L^{6B}, L^{6C}, L^{6D}, and L^{6E} are independently a bond, —NH—, —O—, —S—, —C(O)—, —NHC(O)—, —NHC(O)NH—, —C(O)O—, —OC(O)—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene.

R¹ and R² are independently unsubstituted C₁-C₂₅ alkyl, wherein at least one of R¹ and R² is unsubstituted C₉-C₁₉ alkyl. In embodiments, R¹ and R² are independently unsubstituted C₁-C₂₀alkyl, wherein at least one of R¹ and R² is unsubstituted C₉-C₁₉ alkyl.

R³ is

hydrogen, —NH₂, —OH, —SH, —C(O)H, —C(O)NH₂, —NHC(O)H, —NHC(O)OH, —NHC(O)NH₂, —C(O) OH, —OC(O)H, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

t is an integer from 1 to 5.

In embodiments, t is 1. In embodiments, t is 2. In embodiment, t is 3. In embodiments, t is 4. In embodiment t is 5.

In embodiments, one L³ is attached to a 3′ carbon of the oligonucleotide. In embodiments, one L³ is attached to a 3′ nitrogen of the oligonucleotide (e.g., the 3′ nitrogen of a morpholino moiety). In embodiments, one L³ is attached to a 5′ carbon of the oligonucleotide. In embodiments, one L³ is attached to a 6′ carbon of the oligonucleotide (e.g., the 6′ carbon of a morpholino moiety). In embodiments, one L³ is attached to a 2′ carbon of the oligonucleotide In embodiments, one L³ is attached to a nucleobase of the oligonucleotide.

In embodiments, one L³ is attached to a 3′ carbon of the oligonucleotide at a 3′ end. In embodiments, one L³ is attached to a 3′ nitrogen of the oligonucleotide (e.g., the 3′ nitrogen of a morpholino moiety) at a 3′ end. In embodiments, one L³ is attached to a 5′ carbon of the oligonucleotide at a 5′ end. In embodiments, one L³ is attached to a 6′ carbon of the oligonucleotide (e.g., the 6′ carbon of a morpholino moiety) at a 5′ end.

In embodiments, one L³ is attached to a 3′ carbon of the double-stranded oligonucleotide at either of its 3′ ends. In embodiments, one L³ is attached to a 3′ carbon of the double-stranded oligonucleotide at the 3′end of its antisense strand. In embodiments, one L³ is attached to a 3′ carbon of the double-stranded oligonucleotide at the 3′end of its sense strand.

In embodiments, one L³ is attached to a 3′ nitrogen of the double-stranded oligonucleotide (e.g., the 3′ nitrogen of a morpholino moiety) at either of its 3′ ends. In embodiments, one L³ is attached to a 3′ nitrogen of the double-stranded oligonucleotide (e.g., the 3′ nitrogen of a morpholino moiety) at the 3′end of its antisense strand. In embodiments, one L³ is attached to a 3′ nitrogen of the double-stranded oligonucleotide (e.g., the 3′ nitrogen of a morpholino moiety) at the 3′end of its sense strand.

In embodiments, one L³ is attached to a 5′ carbon of the double-stranded oligonucleotide at either of its 5′ ends. In embodiments, one L³ is attached to a 5′ carbon of the double-stranded oligonucleotide at the 5′ end of its antisense strand. In embodiments, one L³ is attached to a 5′ carbon of the double-stranded oligonucleotide at the 5′ end of its sense strand.

In embodiments, one L³ is attached to a 6′ carbon of the double-stranded oligonucleotide (e.g., the 6′ carbon of a morpholino moiety) at either of its 5′ ends. In embodiments, one L³ is attached to a 6′ carbon of the double-stranded oligonucleotide (e.g., the 6′ carbon of a morpholino moiety) at the 5′ end of its antisense strand. In embodiments, one L³ is attached to a 6′ carbon of the double-stranded oligonucleotide (e.g., the 6′ carbon of a morpholino moiety) at the 5′ end of its sense strand.

In embodiments, one L³ is attached to a 2′ carbon of the double-stranded oligonucleotide. In embodiments, one L³ is attached to a 2′ carbon of the double-stranded oligonucleotide at either of its 3′ ends. In embodiments, one L³ is attached to a 2′ carbon of the double-stranded oligonucleotide at the 3′end of its antisense strand. In embodiments, one L³ is attached to a 2′ carbon of the double-stranded oligonucleotide at the 3′end of its sense strand. In embodiments, one L³ is attached to a 2′ carbon of the double-stranded oligonucleotide at either of its 5′ ends. In embodiments, one L³ is attached to a 2′ carbon of the double-stranded oligonucleotide at the 5′ end of its antisense strand. In embodiments, one L³ is attached to a 2′ carbon of the double-stranded oligonucleotide at the 5′ end of its sense strand.

In embodiments, one L³ is attached to a nucleobase of the double-stranded oligonucleotide. In embodiments, one L³ is attached to a a nucleobase of the double-stranded oligonucleotide at either of its 3′ ends. In embodiments, one L³ is attached to a nucleobase of the double-stranded oligonucleotide at the 3′end of its antisense strand. In embodiments, one L³ is attached to a nucleobase of the double-stranded oligonucleotide at the 3′end of its sense strand. In embodiments, one L³ is attached to a nucleobase of the double-stranded oligonucleotide at either of its 5′ ends. In embodiments, one L³ is attached to a nucleobase of the double-stranded oligonucleotide at the 5′ end of its antisense strand. In embodiments, one L is attached to a nucleobase of the double-stranded oligonucleotide at the 5′ end of its sense strand.

In embodiments, one L³ is attached to a 3′ carbon of the single-stranded oligonucleotide at the 3′ end. In embodiments, one L³ is attached to a 3′ nitrogen of the single-stranded oligonucleotide (e.g., the 3′ nitrogen of a morpholino moiety) at the 3′ end of the single-stranded oligonucleotide.

In embodiments, one L³ is attached to a 5′ carbon of the single-stranded oligonucleotide at the 5′ end.

In embodiments, one L³ is attached to a 6′ carbon of the single-stranded oligonucleotide (e.g., the 6′ carbon of a morpholino moiety) at the 5′ end.

In embodiments, one L³ is attached to a 2′ carbon of the single-stranded oligonucleotide. In embodiments, one L³ is attached to a 2′ carbon of the single-stranded oligonucleotide at the 5′ end. In embodiments, one L³ is attached to a 2′ carbon of the single-stranded oligonucleotide at the 3′ end.

In embodiments, one L³ is attached to a nucleobase of the single-stranded oligonucleotide. In embodiments, one L³ is attached to a nucleobase of the single-stranded oligonucleotide at the of 3′ end. In embodiments, one L³ is attached to a nucleobase of the single-stranded oligonucleotide at the 5′ end.

In embodiments, L³ is a bond, —N(R²³)—, —O—, —S—, —C(O)—, —N(R²³)C(O)—, —C(O)N(R²⁴)—, —N(R²³)C(O)N(R²⁴)—, —C(O)O—, —OC(O)—, —N(R²³)C(O)O—, —OC(O)N(R²⁴)—, —OPO₂—O—, —O—P(O)(S)—O—, —O—P(O)(R²⁵)—O—, —O—P(S)(R²⁵)—O—, —O—P(O)(NR²³R²⁴)—N—, —O—P(S)(NR²³R²⁴)—N—, —O—P(O)(NR²³R²⁴)—O—, —O—P(S)(NR²³R²⁴)—O—, —P(O)(NR²³R²⁴)—N—, —P(S)(NR²³R²⁴)—N—, —P(O)(NR²³R²⁴)—O—, —P(S)(NR²³R²⁴)—O—, —S—S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, L³ is a bond. In embodiments, L³ is —N(R²³)—. In embodiments, L³ is —O— or —S—. In embodiments, L³ is —C(O)—. In embodiments, L³ is —N(R²³)C(O)— or —C(O)N(R²⁴)—. In embodiments, L³ is —N(R²³)C(O)N(R²⁴)—. In embodiments, L³ is —C(O)O— or —OC(O)—. In embodiments, L³ is —N(R²³)C(O)O— or —OC(O)N(R²⁴)—. In embodiments, L³ is —OPO₂—O—, —O—P(O)(S)—O—, —O—P(O)(R²⁵)—O—, —O—P(O)(NR²³R²⁴)—N—, or O—P(O)(NR²³R²⁴)—O—. In embodiments, L³ is —P(O)(NR²³R²⁴)—N—, —P(S)(NR²³R²⁴)—N—, —P(O)(NR²³R²⁴)—O— or —P(S)(NR²³R²⁴)—O—. In embodiments, L³ is —S—S—.

In embodiments, L³ is independently substituted or unsubstituted alkylene (e.g., C₁-C₂₃, C₁-C₁₂, C₁-C₈, C₁-C₆, C₁-C₄, or C₁-C₂). In embodiments, L³ is independently substituted alkylene (e.g., C₁-C₂₃, C₁-C₁₂, C₁-C₈, C₁-C₆, C₁-C₄, or C₁-C₂). In embodiments, L³ is independently unsubstituted alkylene (e.g., C₁-C₂₃, C₁-C₁₂, C₁-C₈, C₁-C₆, C₁-C₄, or C₁-C₂). In embodiments, L³ is independently substituted or unsubstituted C₁-C₂₃ alkylene. In embodiments, L³ is independently substituted C₁-C₂₃ alkylene. In embodiments, L³ is independently unsubstituted C₁-C₂₃ alkylene. In embodiments, L³ is independently substituted or unsubstituted C₁-C₁₂ alkylene. In embodiments, L³ is independently substituted C₁-C₁₂ alkylene. In embodiments, L³ is independently unsubstituted C₁-C₁₂ alkylene. In embodiments, L³ is independently substituted or unsubstituted C₁-C₈ alkylene. In embodiments, L³ is independently substituted C₁-C₈ alkylene. In embodiments, L³ is independently unsubstituted C₁-C₈ alkylene. In embodiments, L³ is independently substituted or unsubstituted C₁-C₆ alkylene. In embodiments, L³ is independently substituted C₁-C₆ alkylene. In embodiments, L³ is independently unsubstituted C₁-C₆ alkylene. In embodiments, L³ is independently substituted or unsubstituted C₁-C₄ alkylene. In embodiments, L³ is independently substituted C₁-C₄ alkylene. In embodiments, L³ is independently unsubstituted C₁-C₄ alkylene. In embodiments, L³ is independently substituted or unsubstituted ethylene. In embodiments, L³ is independently substituted ethylene. In embodiments, L³ is independently unsubstituted ethylene. In embodiments, L³ is independently substituted or unsubstituted methylene. In embodiments, L³ is independently substituted methylene. In embodiments, L³ is independently unsubstituted methylene.

In embodiments, L³ is independently substituted or unsubstituted heteroalkylene (e.g., 2 to 23 membered, 2 to 12 membered, 2 to 8 membered, 2 to 6 membered, 4 to 6 membered, 2 to 3 membered, or 4 to 5 membered). In embodiments, L³ is independently substituted heteroalkylene (e.g., 2 to 23 membered, 2 to 12 membered, 2 to 8 membered, 2 to 6 membered, 4 to 6 membered, 2 to 3 membered, or 4 to 5 membered). In embodiments, L³ is independently unsubstituted heteroalkylene (e.g., 2 to 23 membered, 2 to 12 membered, 2 to 8 membered, 2 to 6 membered, 4 to 6 membered, 2 to 3 membered, or 4 to 5 membered). In embodiments, L³ is independently substituted or unsubstituted 2 to 23 membered heteroalkylene. In embodiments, L³ is independently substituted 2 to 23 membered heteroalkylene. In embodiments, L³ is independently unsubstituted 2 to 23 membered heteroalkylene. In embodiments, L³ is independently substituted or unsubstituted 2 to 8 membered heteroalkylene. In embodiments, L³ is independently substituted 2 to 8 membered heteroalkylene. In embodiments, L³ is independently unsubstituted 2 to 8 membered heteroalkylene. In embodiments, L³ is independently substituted or unsubstituted 2 to 6 membered heteroalkylene. In embodiments, L³ is independently substituted 2 to 6 membered heteroalkylene. In embodiments, L³ is independently unsubstituted 2 to 6 membered heteroalkylene. In embodiments, L³ is independently substituted or unsubstituted 4 to 6 membered heteroalkylene. In embodiments, L³ is independently substituted 4 to 6 membered heteroalkylene. In embodiments, L³ is independently unsubstituted 4 to 6 membered heteroalkylene. In embodiments, L³ is independently substituted or unsubstituted 2 to 3 membered heteroalkylene. In embodiments, L³ is independently substituted 2 to 3 membered heteroalkylene. In embodiments, L³ is independently unsubstituted 2 to 3 membered heteroalkylene. In embodiments, L³ is independently substituted or unsubstituted 4 to 5 membered heteroalkylene. In embodiments, L³ is independently substituted 4 to 5 membered heteroalkylene. In embodiments, L³ is independently unsubstituted 4 to 5 membered heteroalkylene.

In embodiments, L⁴ is a bond, —N(R²³)—, —O—, —S—, —C(O)—, —N(R²³)C(O)—, —C(O)N(R²⁴)—, —N(R²³)C(O)N(R²⁴)—, —C(O)O—, —OC(O)—, —N(R²³)C(O)O—, —OC(O)N(R²⁴)—, —OPO₂—O—, —O—P(O)(S)—O—, —O—P(O)(R²⁵)—O—, —O—P(S)(R²⁵)—O—, —O—P(O)(NR²³R²⁴)—N—, —O—P(S)(NR²³R²⁴)—N—, —O—P(O)(NR²³R²⁴)—O—, —O—P(S)(NR²³R²⁴)—O—, —P(O)(NR²³R²⁴)—N—, —P(S)(NR²³R²⁴)—N—, —P(O)(NR²³R²⁴)—O—, —P(S)(NR²³R²⁴)—O—, —S—S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, L⁴ is a bond. In embodiments, L⁴ is —N(R²³)—. In embodiments, L⁴ is —O— or —S—. In embodiments, L⁴ is —C(O)—. In embodiments, L⁴ is —N(R²³)C(O)— or —C(O)N(R²⁴)—. In embodiments, L⁴ is —N(R²³)C(O)N(R²⁴)—. In embodiments, L⁴ is —C(O)O— or —OC(O)—. In embodiments, L⁴ is —N(R²³)C(O)O— or —OC(O)N(R²⁴)—. In embodiments, L⁴ is —OPO₂—O—, —O—P(O)(S)—O—, —O—P(O)(R²⁵)—O—, —O—P(O)(NR²³R²⁴)—N—, or O—P(O)(NR²³R²⁴)—O—. In embodiments, L⁴ is —P(O)(NR²³R²⁴)—N—, —P(S)(NR²³R²⁴)—N—, —P(O)(NR²³R²⁴)—O— or —P(S)(NR²³R²⁴)—O—. In embodiments, L⁴ is —S—S—.

In embodiments, L⁴ is independently substituted or unsubstituted alkylene (e.g., C₁-C₂₃, C₁-C₁₂, C₁-C₈, C₁-C₆, C₁-C₄, or C₁-C₂). In embodiments, L⁴ is independently substituted alkylene (e.g., C₁-C₂₃, C₁-C₁₂, C₁-C₈, C₁-C₆, C₁-C₄, or C₁-C₂). In embodiments, L⁴ is independently unsubstituted alkylene (e.g., C₁-C₂₃, C₁-C₁₂, C₁-C₈, C₁-C₆, C₁-C₄, or C₁-C₂). In embodiments, L⁴ is independently substituted or unsubstituted C₁-C₂₃ alkylene. In embodiments, L⁴ is independently substituted C₁-C₂₃ alkylene. In embodiments, L⁴ is independently unsubstituted C₁-C₂₃ alkylene. In embodiments, L⁴ is independently substituted or unsubstituted C₁-C₁₂ alkylene. In embodiments, L⁴ is independently substituted C₁-C₁₂ alkylene. In embodiments, L⁴ is independently unsubstituted C₁-C₁₂ alkylene. In embodiments, L⁴ is independently substituted or unsubstituted C₁-C₈ alkylene. In embodiments, L⁴ is independently substituted C₁-C₈ alkylene. In embodiments, L⁴ is independently unsubstituted C₁-C₈ alkylene. In embodiments, L⁴ is independently substituted or unsubstituted C₁-C₆ alkylene. In embodiments, L⁴ is independently substituted C₁-C₆ alkylene. In embodiments, L⁴ is independently unsubstituted C₁-C₆ alkylene. In embodiments, L⁴ is independently substituted or unsubstituted C₁-C₄ alkylene. In embodiments, L⁴ is independently substituted C₁-C₄ alkylene. In embodiments, L⁴ is independently unsubstituted C₁-C₄ alkylene. In embodiments, L⁴ is independently substituted or unsubstituted ethylene. In embodiments, L⁴ is independently substituted ethylene. In embodiments, L⁴ is independently unsubstituted ethylene. In embodiments, L⁴ is independently substituted or unsubstituted methylene. In embodiments, L⁴ is independently substituted methylene. In embodiments, L⁴ is independently unsubstituted methylene.

In embodiments, L⁴ is independently substituted or unsubstituted heteroalkylene (e.g., 2 to 23 membered, 2 to 12 membered, 2 to 8 membered, 2 to 6 membered, 4 to 6 membered, 2 to 3 membered, or 4 to 5 membered). In embodiments, L⁴ is independently substituted heteroalkylene (e.g., 2 to 23 membered, 2 to 12 membered, 2 to 8 membered, 2 to 6 membered, 4 to 6 membered, 2 to 3 membered, or 4 to 5 membered). In embodiments, L⁴ is independently unsubstituted heteroalkylene (e.g., 2 to 23 membered, 2 to 12 membered, 2 to 8 membered, 2 to 6 membered, 4 to 6 membered, 2 to 3 membered, or 4 to 5 membered). In embodiments, L⁴ is independently substituted or unsubstituted 2 to 23 membered heteroalkylene. In embodiments, L⁴ is independently substituted 2 to 23 membered heteroalkylene. In embodiments, L⁴ is independently unsubstituted 2 to 23 membered heteroalkylene. In embodiments, L⁴ is independently substituted or unsubstituted 2 to 8 membered heteroalkylene. In embodiments, L⁴ is independently substituted 2 to 8 membered heteroalkylene. In embodiments, L⁴ is independently unsubstituted 2 to 8 membered heteroalkylene. In embodiments, L⁴ is independently substituted or unsubstituted 2 to 6 membered heteroalkylene. In embodiments, L⁴ is independently substituted 2 to 6 membered heteroalkylene. In embodiments, L⁴ is independently unsubstituted 2 to 6 membered heteroalkylene. In embodiments, L⁴ is independently substituted or unsubstituted 4 to 6 membered heteroalkylene. In embodiments, L⁴ is independently substituted 4 to 6 membered heteroalkylene. In embodiments, L⁴ is independently unsubstituted 4 to 6 membered heteroalkylene. In embodiments, L⁴ is independently substituted or unsubstituted 2 to 3 membered heteroalkylene. In embodiments, L⁴ is independently substituted 2 to 3 membered heteroalkylene. In embodiments, L⁴ is independently unsubstituted 2 to 3 membered heteroalkylene. In embodiments, L⁴ is independently substituted or unsubstituted 4 to 5 membered heteroalkylene. In embodiments, L⁴ is independently substituted 4 to 5 membered heteroalkylene. In embodiments, L⁴ is independently unsubstituted 4 to 5 membered heteroalkylene.

R²³ is independently hydrogen or unsubstituted alkyl (e.g., C₁-C₂₃, C₁-C₁₂, C₁-C₈, C₁-C₆, C₁-C₄, or C₁-C₂). In embodiments, R²³ is independently hydrogen. In embodiments, R²³ is independently unsubstituted C₁-C₂₃ alkyl. In embodiments, R²³ is independently hydrogen or unsubstituted C₁-C₁₂ alkyl. In embodiments, R²³ is independently hydrogen or unsubstituted C₁-C₁₀ alkyl. In embodiments, R²³ is independently hydrogen or unsubstituted C₁-C₈ alkyl. In embodiments, R²³ is independently hydrogen or unsubstituted C₁-C₆ alkyl. In embodiments, R²³ is independently hydrogen or unsubstituted C₁-C₄ alkyl. In embodiments, R²³ is independently hydrogen or unsubstituted C₁-C₂ alkyl.

R²⁴ is independently hydrogen or unsubstituted alkyl (e.g., C₁-C₂₄, C₁-C₁₂, C₁-C₈, C₁-C₆, C₁-C₄, or C₁-C₂). In embodiments, R²⁴ is independently hydrogen. In embodiments, R²⁴ is independently unsubstituted C₁-C₂₄ alkyl. In embodiments, R²⁴ is independently hydrogen or unsubstituted C₁-C₁₂ alkyl. In embodiments, R²⁴ is independently hydrogen or unsubstituted C₁-C₁₀ alkyl. In embodiments, R²⁴ is independently hydrogen or unsubstituted C₁-C₈ alkyl. In embodiments, R²⁴ is independently hydrogen or unsubstituted C₁-C₆ alkyl. In embodiments, R²⁴ is independently hydrogen or unsubstituted C₁-C₄ alkyl. In embodiments, R²⁴ is independently hydrogen or unsubstituted C₁-C₂ alkyl.

R²⁵ is independently hydrogen or unsubstituted alkyl (e.g., C₁-C₂₅, C₁-C₁₂, C₁-C₈, C₁-C₆, C₁-C₄, or C₁-C₂). In embodiments, R²⁵ is independently hydrogen. In embodiments, R²⁵ is independently unsubstituted C₁-C₂₅ alkyl. In embodiments, R²⁵ is independently hydrogen or unsubstituted C₁-C₁₂ alkyl. In embodiments, R²⁵ is independently hydrogen or unsubstituted C₁-C₁₀ alkyl. In embodiments, R²⁵ is independently hydrogen or unsubstituted C₁-C₈ alkyl. In embodiments, R²⁵ is independently hydrogen or unsubstituted C₁-C₆ alkyl. In embodiments, R²⁵ is independently hydrogen or unsubstituted C₁-C₄ alkyl. In embodiments, R²⁵ is independently hydrogen or unsubstituted C₁-C₂ alkyl.

In embodiments, L³ and L⁴ are independently a

bond, —NH—, —O—, —C(O)—, —C(O)O—, —OC(O)—, —OPO₂—O—, —O—P(O)(S)—O—, —O—P(O)(CH₃)—O—, —O—P(S)(CH₃)—O—, —O—P(O)(N(CH₃)₂)—N—, —O—P(O)(N(CH₃)₂)—O—, —O—P(S)(N(CH₃)₂)—N—, —O—P(S)(N(CH₃)₂)—O—, —P(O)(N(CH₃)₂)—N—, —P(O)(N(CH₃)₂)—O—, —P(S)(N(CH₃)₂)—N—, —P(S)(N(CH₃)₂)—O—, substituted or unsubstituted alkylene or substituted or unsubstituted heteroalkylene. In embodiments, L³ is independently a
bond, —NH—, —O—, —C(O)—, —C(O)O—, —OC(O)—, —OPO₂—O—, —O—P(O)(S)—O—, —O—P(O)(CH₃)—O—, —O—P(S)(CH₃)—O—, —O—P(O)(N(CH₃)₂)—N—, —O—P(O)(N(CH₃)₂)—O—, —O—P(S)(N(CH₃)₂)—N—, —O—P(S)(N(CH₃)₂)—O—, —P(O)(N(CH₃)₂)—N—, —P(O)(N(CH₃)₂)—O—, —P(S)(N(CH₃)₂)—N—, —P(S)(N(CH₃)₂)—O—, substituted or unsubstituted alkylene or substituted or unsubstituted heteroalkylene. In embodiments, L⁴ is independently a
bond, —NH—, —O—, —C(O)—, —C(O)O—, —OC(O)—, —OPO₂—O—, —O—P(O)(S)—O—, —O—P(O)(CH₃)—O—, —O—P(S)(CH₃)—O—, —O—P(O)(N(CH₃)₂)—N—, —O—P(O)(N(CH₃)₂)—O—, —O—P(S)(N(CH₃)₂)—N—, —O—P(S)(N(CH₃)₂)—O—, —P(O)(N(CH₃)₂)—N—, —P(O)(N(CH₃)₂)—O—, —P(S)(N(CH₃)₂)—N—, —P(S)(N(CH₃)₂)—O—, substituted or unsubstituted alkylene or substituted or unsubstituted heteroalkylene.

In embodiments, L³ is independently

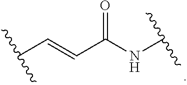 [SMILES: */C=C/C(=O)N*]

In embodiments, L³ is independently —OPO₂—O—. In embodiments, L³ is independently —O—P(O)(S)—O—. In embodiments, L³ is independently —O—. In embodiments, L³ is independently —S—.

In embodiments, L³ is attached to the 3′nitrogen of a morpholino moiety. In embodiments, L³ is independently —C(O)—. In embodiments, L³ is attached to the 6′ carbon of a morpholino moiety. In embodiments, L³ is independently —O—P(O)(N(CH₃)₂)—N—. In embodiments, L³ is independently —O—P(O)(N(CH₃)₂)—O—. In embodiments, L³ is independently —P(O)(N(CH₃)₂)—N—. In embodiments, L³ is independently —P(O)(N(CH₃)₂)—O—.

In embodiments, L⁴ is independently substituted or unsubstituted alkylene or substituted or unsubstituted heteroalkylene. In embodiments, L⁴ is independently -L⁷-NH—C(O)— or -L⁷-C(O)—NH—. In embodiments, L⁷ is independently substituted or unsubstituted alkylene (e.g., C₁-C₂₀, C₁-C₁₂, C₁-C₈, C₁-C₆, C₁-C₄, or C₁-C₂). In embodiments, L⁷ is independently substituted alkylene (e.g., C₁-C₂₀, C₁-C₁₂, C₁-C₈, C₁-C₆, C₁-C₄, or C₁-C₂). In embodiments, L⁷ is independently unsubstituted alkylene (e.g., C₁-C₂₀, C₁-C₁₂, C₁-C₈, C₁-C₆, C₁-C₄, or C₁-C₂).

In embodiments, L⁴ is independently substituted or unsubstituted heteroalkylene (e.g., 2 to 20 membered, 2 to 12 membered, 2 to 10 membered, 2 to 8 membered, 2 to 6 membered, or 2 to 4 membered). In embodiments, L⁴ is independently substituted heteroalkylene (e.g., 2 to 20 membered, 2 to 12 membered, 2 to 10 membered, 2 to 8 membered, 2 to 6 membered, or 2 to 4 membered). In embodiments, L⁴ is independently oxo-substituted heteroalkylene (e.g., 2 to 20 membered, 2 to 12 membered, 2 to 10 membered, 2 to 8 membered, 2 to 6 membered, or 2 to 4 membered). In embodiments, L⁴ is independently unsubstituted heteroalkylene (e.g., 2 to 20 membered, 2 to 12 membered, 2 to 10 membered, 2 to 8 membered, 2 to 6 membered, or 2 to 4 membered).

In embodiments, L⁴ is independently -L⁷-NH—C(O)— or -L⁷-C(O)—NH—; and L⁷ is independently substituted or unsubstituted alkylene (e.g., C₁-C₂₀, C₁-C₁₂, C₁-C₈, C₁-C₆, C₁-C₄, or C₁-C₂). In embodiments, L⁴ is independently -L⁷-NH—C(O)—; and L⁷ is independently substituted or unsubstituted alkylene (e.g., C₁-C₂₀, C₁-C₁₂, C₁-C₈, C₁-C₆, C₁-C₄, or C₁-C₂). In embodiments, L⁴ is independently -L⁷-C(O)—NH—; and L⁷ is independently substituted or unsubstituted alkylene (e.g., C₁-C₂₀, C₁-C₁₂, C₁-C₈, C₁-C₆, C₁-C₄, or C₁-C₂).

In embodiments, L⁷ is independently substituted or unsubstituted alkylene (e.g., C₁-C₂₀, C₁-C₁₂, C₁-C₈, C₁-C₆, C₁-C₄, or C₁-C₂). In embodiments, L⁷ is independently substituted alkylene (e.g., C₁-C₂₀, C₁-C₁₂, C₁-C₈, C₁-C₆, C₁-C₄, or C₁-C₂). In embodiments, L⁷ is independently unsubstituted alkylene (e.g., C₁-C₂₀, C₁-C₁₂, C₁-C₈, C₁-C₆, C₁-C₄, or C₁-C₂). In embodiments, L⁷ is independently substituted or unsubstituted C₁-C₂₀ alkylene. In embodiments, L⁷ is independently substituted C₁-C₂₀ alkylene. In embodiments, L⁷ is independently hydroxy(OH)-substituted C₁-C₂₀ alkylene. In embodiments, L⁷ is independently hydroxymethyl-substituted C₁-C₂₀ alkylene. In embodiments, L⁷ is independently unsubstituted C₁-C₂₀ alkylene. In embodiments, L⁷ is independently substituted or unsubstituted C₁-C₁₂ alkylene. In embodiments, L⁷ is independently substituted C₁-C₁₂ alkylene. In embodiments, L⁷ is independently hydroxy(OH)-substituted C₁-C₁₂ alkylene. In embodiments, L⁷ is independently hydroxymethyl-substituted C₁-C₁₂ alkylene. In embodiments, L⁷ is independently unsubstituted C₁-C₁₂ alkylene. In embodiments, L⁷ is independently substituted or unsubstituted C₁-C₈ alkylene. In embodiments, L⁷ is independently substituted C₁-C₈ alkylene. In embodiments, L⁷ is independently hydroxy(OH)-substituted C₁-C₈ alkylene. In embodiments, L⁷ is independently hydroxymethyl-substituted C₁-C₈ alkylene. In embodiments, L⁷ is independently unsubstituted C₁-C₈ alkylene. In embodiments, L⁷ is independently substituted or unsubstituted C₁-C₆ alkylene. In embodiments, L⁷ is independently substituted C₁-C₆ alkylene. In embodiments, L⁷ is independently hydroxy(OH)-substituted C₁-C₆ alkylene. In embodiments, L⁷ is independently hydroxymethyl-substituted C₁-C₆ alkylene. In embodiments, L⁷ is independently unsubstituted C₁-C₆ alkylene. In embodiments, L⁷ is independently substituted or unsubstituted C₁-C₄ alkylene. In embodiments, L⁷ is independently substituted C₁-C₄ alkylene. In embodiments, L⁷ is independently hydroxy(OH)-substituted C₁-C₄ alkylene. In embodiments, L⁷ is independently hydroxymethyl-substituted C₁-C₄ alkylene. In embodiments, L⁷ is independently unsubstituted C₁-C₄ alkylene. In embodiments, L⁷ is independently substituted or unsubstituted C₁-C₂ alkylene. In embodiments, L⁷ is independently substituted C₁-C₂ alkylene. In embodiments, L⁷ is independently hydroxy(OH)-substituted C₁-C₂ alkylene. In embodiments, L⁷ is independently hydroxymethyl-substituted C₁-C₂ alkylene. In embodiments, L⁷ is independently unsubstituted C₁-C₂ alkylene.

In embodiments, L⁴ is independently -L⁷-NH—C(O)— or -L⁷-C(O)—NH—; and L⁷ is independently substituted or unsubstituted alkylene (e.g., C₁-C₂₀, C₁-C₁₂, C₁-C₈, C₁-C₆, C₁-C₄, or C₁-C₂). In embodiments, L⁴ is independently -L⁷-NH—C(O)— or -L⁷-C(O)—NH—; and L⁷ is independently substituted or unsubstituted C₁-C₈ alkylene. In embodiments, L⁴ is independently -L⁷-NH—C(O)— or -L⁷-C(O)—NH—; and L⁷ is independently substituted C₁-C₈ alkylene. In embodiments, L⁴ is independently -L⁷-NH—C(O)— or -L⁷-C(O)—NH—; and L⁷ is independently hydroxy(OH)-substituted C₁-C₈ alkylene. In embodiments, L⁴ is independently -L⁷-NH—C(O)— or -L⁷-C(O)—NH—; and L⁷ is independently hydroxymethyl-substituted C₁-C₈ alkylene. In embodiments, L⁴ is independently -L⁷-NH—C(O)— or -L⁷-C(O)—NH—; and L⁷ is independently unsubstituted C₁-C₈ alkylene.

In embodiments, L⁴ is independently -L⁷-NH—C(O)— or -L⁷-C(O)—NH—; and L⁷ is independently substituted or unsubstituted C₃-C₈ alkylene. In embodiments, L⁴ is independently -L⁷-NH—C(O)— or -L⁷-C(O)—NH—; and L⁷ is independently substituted C₃-C₈ alkylene. In embodiments, L⁴ is independently -L⁷-NH—C(O)— or -L⁷-C(O)—NH—; and L⁷ is independently hydroxy(OH)-substituted C₃-C₈ alkylene. In embodiments, L⁴ is independently -L⁷-NH—C(O)— or -L⁷-C(O)—NH—; and L⁷ is independently hydroxymethyl-substituted C₃-C₈ alkylene. In embodiments, L⁴ is independently -L⁷-NH—C(O)— or -L⁷-C(O)—NH—; and L⁷ is independently unsubstituted C₃-C₈ alkylene.

In embodiments, L⁴ is independently -L⁷-NH—C(O)— or -L⁷-C(O)—NH—; and L⁷ is independently substituted or unsubstituted C₅-C₈ alkylene. In embodiments, L⁴ is independently -L⁷-NH—C(O)— or -L⁷-C(O)—NH—; and L⁷ is independently substituted C₅-C₈ alkylene. In embodiments, L⁴ is independently -L⁷-NH—C(O)— or -L⁷-C(O)—NH—; and L⁷ is independently hydroxy(OH)-substituted C₅-C₈ alkylene. In embodiments, L⁴ is independently -L⁷-NH—C(O)— or -L⁷-C(O)—NH—; and L⁷ is independently hydroxymethyl-substituted C₅-C₈ alkylene. In embodiments, L⁴ is independently -L⁷-NH—C(O)— or -L⁷-C(O)—NH—; and L⁷ is independently unsubstituted C₅-C₈ alkylene.

In embodiments, L⁴ is independently -L⁷-NH—C(O)— or -L⁷-C(O)—NH—; and L⁷ is independently substituted or unsubstituted octylene. In embodiments, L⁴ is independently -L⁷-NH—C(O)— or -L⁷-C(O)—NH—; and L⁷ is independently substituted octylene. In embodiments, L⁴ is independently -L⁷-NH—C(O)— or -L⁷-C(O)—NH—; and L⁷ is independently hydroxy(OH)-substituted octylene. In embodiments, L⁴ is independently -L⁷-NH—C(O)— or -L⁷-C(O)—NH—; and L⁷ is independently unsubstituted octylene. In embodiments, L⁴ is independently -L⁷-NH—C(O)— and L⁷ is independently hydroxy(OH)-substituted octylene. In embodiments, L⁴ is independently -L⁷-NH—C(O)— and L⁷ is independently hydroxymethyl-substituted octylene. In embodiments, L⁴ is independently -L⁷-NH—C(O)— and L⁷ is independently unsubstituted octylene.

In embodiments, L⁴ is independently -L⁷-NH—C(O)— or -L⁷-C(O)—NH—; and L⁷ is independently substituted or unsubstituted heptylene. In embodiments, L⁴ is independently -L⁷-NH—C(O)— or -L⁷-C(O)—NH—; and L⁷ is independently substituted heptylene. In embodiments, L⁴ is independently -L⁷-NH—C(O)— or -L⁷-C(O)—NH—; and L⁷ is independently hydroxy(OH)-substituted heptylene. In embodiments, L⁴ is independently -L⁷-NH—C(O)— or -L⁷-C(O)—NH—; and L⁷ is independently unsubstituted heptylene. In embodiments, L⁴ is independently -L⁷-NH—C(O)— and L⁷ is independently hydroxy(OH)-substituted heptylene. In embodiments, L⁴ is independently -L⁷-NH—C(O)— and L⁷ is independently hydroxymethyl-substituted heptylene. In embodiments, L⁴ is independently -L⁷-NH—C(O)— and L⁷ is independently unsubstituted heptylene.

In embodiments, L⁴ is independently -L⁷-NH—C(O)— or -L⁷-C(O)—NH—; and L⁷ is independently substituted or unsubstituted hexylene. In embodiments, L⁴ is independently -L⁷-NH—C(O)— or -L⁷-C(O)—NH—; and L⁷ is independently substituted hexylene. In embodiments, L⁴ is independently -L⁷-NH—C(O)— or -L⁷-C(O)—NH—; and L⁷ is independently hydroxy(OH)-substituted hexylene. In embodiments, L⁴ is independently -L⁷-NH—C(O)— or -L⁷-C(O)—NH—; and L⁷ is independently unsubstituted hexylene. In embodiments, L⁴ is independently -L⁷-NH—C(O)— and L⁷ is independently hydroxy(OH)-substituted hexylene. In embodiments, L⁴ is independently -L⁷-NH—C(O)— and L⁷ is independently hydroxymethyl-substituted hexylene. In embodiments, L⁴ is independently -L⁷-NH—C(O)— and L⁷ is independently unsubstituted hexylene.

In embodiments, L⁴ is independently -L⁷-NH—C(O)— or -L⁷-C(O)—NH—; and L⁷ is independently substituted or unsubstituted pentylene. In embodiments, L⁴ is independently -L⁷-NH—C(O)— or -L⁷-C(O)—NH—; and L⁷ is independently substituted pentylene. In embodiments, L⁴ is independently -L⁷-NH—C(O)— or -L⁷-C(O)—NH—; and L⁷ is independently hydroxy(OH)-substituted pentylene. In embodiments, L⁴ is independently -L⁷-NH—C(O)— or -L⁷-C(O)—NH—; and L⁷ is independently unsubstituted pentylene. In embodiments, L⁴ is independently -L⁷-NH—C(O)— and L⁷ is independently hydroxy(OH)-substituted pentylene. In embodiments, L⁴ is independently -L⁷-NH—C(O)— and L⁷ is independently hydroxymethyl-substituted pentylene. In embodiments, L⁴ is independently -L⁷-NH—C(O)— and L⁷ is independently unsubstituted pentylene.

In embodiments, L⁴ is independently

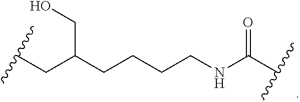 [SMILES: *CC(CO)CCCCNC(*)=O]

In embodiments, L⁴ is independently

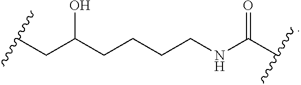 [SMILES: *CC(O)CCCCNC(*)=O]

In embodiments, L⁴ is independently

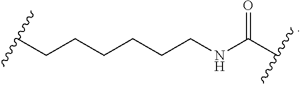 [SMILES: *CCCCCCNC(*)=O]

In embodiments, L⁴ is independently

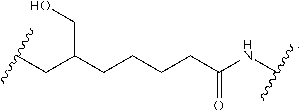 [SMILES: *CC(CO)CCCCC(=O)N*]

In embodiments, L⁴ is independently

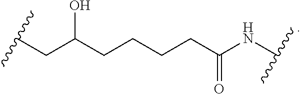 [SMILES: *CC(O)CCCCC(=O)N*]

In embodiments, L⁴ is independently

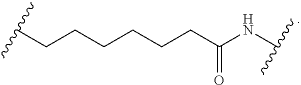 [SMILES: *CCCCCCC(=O)N*]

In embodiments, L⁴ is independently

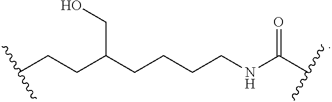 [SMILES: *CCC(CO)CCCCNC(*)=O]

In embodiments, L⁴ is independently

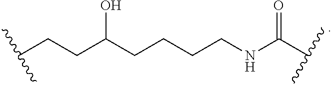 [SMILES: *CCC(O)CCCCNC(*)=O]

In embodiments, L⁴ is independently

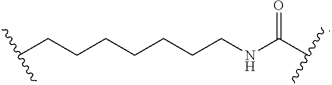 [SMILES: *CCCCCCCNC(*)=O]

In embodiments, L⁴ is independently

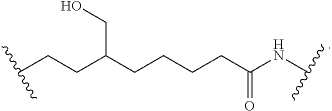 [SMILES: *CCC(CO)CCCCC(=O)N*]

In embodiments, L⁴ is independently

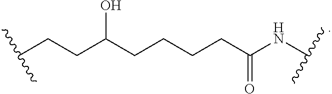 [SMILES: *CCC(O)CCCCC(=O)N*]

In embodiments, L⁴ is independently

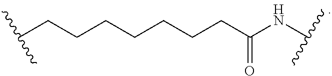 [SMILES: *CCCCCCCC(=O)N*]

In embodiments, -L³-L⁴- is independently -L⁷-NH—C(O)— or -L⁷-C(O)—NH—. In embodiments, L⁷ is independently substituted or unsubstituted heteroalkylene (e.g., 2 to 20 membered, 2 to 12 membered, 2 to 10 membered, 2 to 8 membered, 2 to 6 membered, or 2 to 4 membered). In embodiments, L⁷ is independently substituted heteroalkylene (e.g., 2 to 20 membered, 2 to 12 membered, 2 to 10 membered, 2 to 8 membered, 2 to 6 membered, or 2 to 4 membered). In embodiments, L⁷ is independently oxo-substituted heteroalkylene (e.g., 2 to 20 membered, 2 to 12 membered, 2 to 10 membered, 2 to 8 membered, 2 to 6 membered, or 2 to 4 membered). In embodiments, L⁷ is independently unsubstituted heteroalkylene (e.g., 2 to 20 membered, 2 to 12 membered, 2 to 10 membered, 2 to 8 membered, 2 to 6 membered, or 2 to 4 membered). In embodiments, L⁷ is independently substituted or unsubstituted heteroalkenylene (e.g., 2 to 20 membered, 2 to 12 membered, 2 to 10 membered, 2 to 8 membered, 2 to 6 membered, or 2 to 4 membered). In embodiments, L⁷ is independently substituted heteroalkenylene (e.g., 2 to 20 membered, 2 to 12 membered, 2 to 10 membered, 2 to 8 membered, 2 to 6 membered, or 2 to 4 membered). In embodiments, L⁷ is independently oxo-substituted heteroalkenylene (e.g., 2 to 20 membered, 2 to 12 membered, 2 to 10 membered, 2 to 8 membered, 2 to 6 membered, or 2 to 4 membered). In embodiments, L⁷ is independently unsubstituted heteroalkenylene (e.g., 2 to 20 membered, 2 to 12 membered, 2 to 10 membered, 2 to 8 membered, 2 to 6 membered, or 2 to 4 membered).

In embodiments, L⁷ is independently substituted or unsubstituted 2 to 20 membered heteroalkylene. In embodiments, L⁷ is independently substituted 2 to 20 membered heteroalkylene. In embodiments, L⁷ is independently oxo-substituted 2 to 20 membered heteroalkylene. In embodiments, L⁷ is independently unsubstituted 2 to 20 membered heteroalkylene. In embodiments, L⁷ is independently substituted or unsubstituted 2 to 12 membered heteroalkylene. In embodiments, L⁷ is independently substituted 2 to 12 membered heteroalkylene. In embodiments, L⁷ is independently oxo-substituted 2 to 12 membered heteroalkylene. In embodiments, L⁷ is independently unsubstituted 2 to 12 membered heteroalkylene. In embodiments, L⁷ is independently substituted or unsubstituted 2 to 10 membered heteroalkylene. In embodiments, L⁷ is independently substituted 2 to 10 membered heteroalkylene. In embodiments, L⁷ is independently oxo-substituted 2 to 10 membered heteroalkylene. In embodiments, L⁷ is independently unsubstituted 2 to 10 membered heteroalkylene. In embodiments, L⁷ is independently substituted or unsubstituted 2 to 8 membered heteroalkylene. In embodiments, L⁷ is independently substituted 2 to 8 membered heteroalkylene. In embodiments, L⁷ is independently oxo-substituted 2 to 8 membered heteroalkylene. In embodiments, L⁷ is independently unsubstituted 2 to 8 membered heteroalkylene. In embodiments, L⁷ is independently substituted or unsubstituted 2 to 6 membered heteroalkylene. In embodiments, L⁷ is independently substituted 2 to 6 membered heteroalkylene. In embodiments, L⁷ is independently oxo-substituted 2 to 6 membered heteroalkylene. In embodiments, L⁷ is independently unsubstituted 2 to 6 membered heteroalkylene. In embodiments, L⁷ is independently substituted or unsubstituted 2 to 4 membered heteroalkylene. In embodiments, L⁷ is independently substituted 2 to 4 membered heteroalkylene. In embodiments, L⁷ is independently oxo-substituted 2 to 4 membered heteroalkylene. In embodiments, L⁷ is independently unsubstituted 2 to 4 membered heteroalkylene.

In embodiments, L⁷ is independently substituted or unsubstituted 2 to 20 membered heteroalkenylene. In embodiments, L⁷ is independently substituted 2 to 20 membered heteroalkenylene. In embodiments, L⁷ is independently oxo-substituted 2 to 20 membered heteroalkenylene. In embodiments, L⁷ is independently unsubstituted 2 to 20 membered heteroalkenylene. In embodiments, L⁷ is independently substituted or unsubstituted 2 to 12 membered heteroalkenylene. In embodiments, L⁷ is independently substituted 2 to 12 membered heteroalkenylene. In embodiments, L⁷ is independently oxo-substituted 2 to 12 membered heteroalkenylene. In embodiments, L⁷ is independently unsubstituted 2 to 12 membered heteroalkenylene. In embodiments, L⁷ is independently substituted or unsubstituted 2 to 10 membered heteroalkenylene. In embodiments, L⁷ is independently substituted 2 to 10 membered heteroalkenylene. In embodiments, L⁷ is independently oxo-substituted 2 to 10 membered heteroalkenylene. In embodiments, L⁷ is independently unsubstituted 2 to 10 membered heteroalkenylene. In embodiments, L⁷ is independently substituted or unsubstituted 2 to 8 membered heteroalkenylene. In embodiments, L⁷ is independently substituted 2 to 8 membered heteroalkenylene. In embodiments, L⁷ is independently oxo-substituted 2 to 8 membered heteroalkenylene. In embodiments, L⁷ is independently unsubstituted 2 to 8 membered heteroalkenylene. In embodiments, L⁷ is independently substituted or unsubstituted 2 to 6 membered heteroalkenylene. In embodiments, L⁷ is independently substituted 2 to 6 membered heteroalkenylene. In embodiments, L⁷ is independently oxo-substituted 2 to 6 membered heteroalkenylene. In embodiments, L⁷ is independently unsubstituted 2 to 6 membered heteroalkenylene. In embodiments, L⁷ is independently substituted or unsubstituted 2 to 4 membered heteroalkenylene. In embodiments, L⁷ is independently substituted 2 to 4 membered heteroalkenylene. In embodiments, L⁷ is independently oxo-substituted 2 to 4 membered heteroalkenylene. In embodiments, L⁷ is independently unsubstituted 2 to 4 membered heteroalkenylene.

In embodiments, -L³-L⁴- is independently —O-L⁷-NH—C(O)— or —O-L⁷-C(O)—NH—. In embodiments, L⁷ is independently substituted or unsubstituted alkylene (e.g., C₁-C₂₀, C₁-C₁₂, C₁-C₈, C₁-C₆, C₁-C₄, or C₁-C₂). In embodiments, -L³-L⁴- is independently —O-L⁷-NH—C(O)— or —O-L⁷-C(O)—NH—; and L⁷ is independently substituted or unsubstituted alkylene (e.g., C₁-C₂₀, C₁-C₁₂, C₁-C₈, C₁-C₆, C₁-C₄, or C₁-C₂). In embodiments, -L³-L⁴- is independently —O-L⁷-NH—C(O)—; and L⁷ is independently substituted or unsubstituted alkylene (e.g., C₁-C₂₀, C₁-C₁₂, C₁-C₈, C₁-C₆, C₁-C₄, or C₁-C₂). In embodiments, -L³-L⁴- is independently —O-L⁷-C(O)—NH—; and L⁷ is independently substituted or unsubstituted alkylene (e.g., C₁-C₂₀, C₁-C₁₂, C₁-C₈, C₁-C₆, C₁-C₄, or C₁-C₂).

In embodiments, -L³-L⁴- is independently —O-L⁷-C(O)—NH—; and L⁷ is independently substituted or unsubstituted C₁-C₈ alkylene. In embodiments, -L³-L⁴- is independently —O-L⁷-C(O)—NH—; and L⁷ is independently substituted C₁-C₈ alkylene. In embodiments, -L³-L⁴-is independently —O-L⁷-C(O)—NH—; and L⁷ is independently hydroxy(OH)-substituted C₁-C₈ alkylene. In embodiments, -L³-L⁴- is independently —O-L⁷-C(O)—NH— and L⁷ is independently hydroxymethyl-substituted C₁-C₈ alkylene. In embodiments, -L³-L⁴- is independently —O-L⁷-C(O)—NH—; and L⁷ is independently unsubstituted C₁-C₈ alkylene.

In embodiments, -L³-L⁴-is independently —O-L⁷-C(O)—NH—; and L⁷ is independently substituted or unsubstituted C₃-C₈ alkylene. In embodiments, -L³-L⁴- is independently —O-L⁷-C(O)—NH—; and L⁷ is independently substituted C₃-C₈ alkylene. In embodiments, -L³-L⁴-is independently —O-L⁷-C(O)—NH—; and L⁷ is independently hydroxy(OH)-substituted C₃-C₈ alkylene. In embodiments, -L³-L⁴- is independently —O-L⁷-C(O)—NH— and L⁷ is independently hydroxymethyl-substituted C₃-C₈ alkylene. In embodiments, -L³-L⁴- is independently —O-L⁷-C(O)—NH—; and L⁷ is independently unsubstituted C₃-C₈ alkylene.

In embodiments, -L³-L⁴-is independently —O-L⁷-C(O)—NH—; and L⁷ is independently substituted or unsubstituted C₅-C₈ alkylene. In embodiments, -L³-L⁴- is independently —O-L⁷-C(O)—NH—; and L⁷ is independently substituted C₅-C₈ alkylene. In embodiments, -L³-L⁴-is independently —O-L⁷-C(O)—NH—; and L⁷ is independently hydroxy(OH)-substituted C₅-C₈ alkylene. In embodiments, -L³-L⁴- is independently —O-L⁷-C(O)—NH— and L⁷ is independently hydroxymethyl-substituted C₅-C₈ alkylene. In embodiments, -L³-L⁴- is independently —O-L⁷-C(O)—NH—; and L⁷ is independently unsubstituted C₅-C₈ alkylene.

In embodiments, -L³-L⁴-is independently —O-L⁷-NH—C(O)—; and L⁷ is independently substituted or unsubstituted C₁-C₈ alkylene. In embodiments, -L³-L⁴- is independently —O-L⁷-NH—C(O)—; and L⁷ is independently substituted C₁-C₈ alkylene. In embodiments, -L³-L⁴- is independently —O-L⁷-NH—C(O)—; and L⁷ is independently hydroxy(OH)-substituted C₁-C₈ alkylene. In embodiments, -L³-L⁴- is independently —O-L⁷-NH—C(O)—; and L⁷ is independently hydroxymethyl-substituted C₁-C₈ alkylene. In embodiments, -L³-L⁴- is independently —O-L⁷-NH—C(O)—; and L⁷ is independently unsubstituted C₁-C₈ alkylene.

In embodiments, -L³-L⁴- is independently —O-L⁷-NH—C(O)—; and L⁷ is independently substituted or unsubstituted C₃-C₈ alkylene. In embodiments, -L³-L⁴- is independently —O-L⁷-NH—C(O)—; and L⁷ is independently substituted C₃-C₈ alkylene. In embodiments, -L³-L⁴- is independently —O-L⁷-NH—C(O)—; and L⁷ is independently hydroxy(OH)-substituted C₃-C₈ alkylene. In embodiments, -L³-L⁴- is independently —O-L⁷-NH—C(O)—; and L⁷ is independently hydroxymethyl-substituted C₃-C₈ alkylene. In embodiments, -L³-L⁴- is independently —O-L⁷-NH—C(O)—; and L⁷ is independently unsubstituted C₃-C₈ alkylene.

In embodiments, -L³-L⁴- is independently —O-L⁷-NH—C(O)—; and L⁷ is independently substituted or unsubstituted C₅-C₈ alkylene. In embodiments, -L³-L⁴- is independently —O-L⁷-NH—C(O)—; and L⁷ is independently substituted C₅-C₈ alkylene. In embodiments, -L³-L⁴- is independently —O-L⁷-NH—C(O)—; and L⁷ is independently hydroxy(OH)-substituted C₅-C₈ alkylene. In embodiments, -L³-L⁴- is independently —O-L⁷-NH—C(O)—; and L⁷ is independently hydroxymethyl-substituted C₅-C₈ alkylene. In embodiments, -L³-L⁴- is independently —O-L⁷-NH—C(O)—; and L⁷ is independently unsubstituted C₅-C₈ alkylene.

In embodiments, -L³-L⁴- is independently

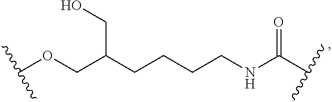 [SMILES: *OCC(CO)CCCCNC(*)=O]

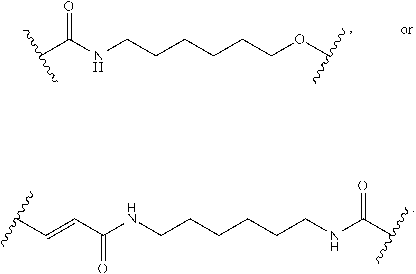 [SMILES: *OCCCCCCNC(*)=O ; */C=C/C(=O)NCCCCCCNC(*)=O]

In embodiments, -L³-L⁴- is independently

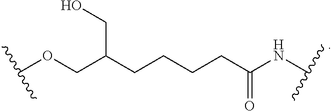 [SMILES: *NC(=O)CCCCC(CO)CO*]

In embodiments, -L³-L⁴- is independently

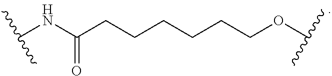 [SMILES: *NC(=O)CCCCCCO*]

In embodiments, -L³-L⁴- is independently

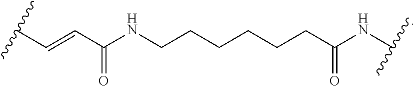 [SMILES: */C=C/C(=O)NCCCCCCC(=O)N*]

In embodiments, -L³-L⁴- is independently —OPO₂—O-L⁷-NH—C(O)—, —OP(O)(S)—O-L⁷-NH—C(O)—, —OPO₂—O-L⁷-C(O)—NH— or —OP(O)(S)—O-L⁷-C(O)—NH—. In embodiments, L⁷ is independently substituted or unsubstituted alkylene (e.g., C₁-C₂₀, C₁-C₁₂, C₁-C₈, C₁-C₆, C₁-C₄, or C₁-C₂). In embodiments, -L³-L⁴- is independently —OPO₂—O-L⁷-NH—C(O)— or —OP(O)(S)—O-L⁷-NH—C(O)—; and L⁷ is independently substituted or unsubstituted alkylene. In embodiments, -L³-L⁴- is independently —OPO₂—O-L⁷-NH—C(O)—; and L⁷ is independently substituted or unsubstituted alkylene. In embodiments, -L³-L⁴- is independently —OP(O)(S)—O-L⁷-NH—C(O)—; and L⁷ is independently substituted or unsubstituted alkylene. In embodiments, -L³-L⁴- is independently —OPO₂—O-L⁷-C(O)—NH— or —OP(O)(S)—O-L⁷-C(O)—NH—; and L⁷ is independently substituted or unsubstituted alkylene. In embodiments, -L³-L⁴- is independently —OPO₂—O-L⁷-C(O)—NH—; and L⁷ is independently substituted or unsubstituted alkylene. In embodiments, -L³-L⁴-is independently —OP(O)(S)—O-L⁷-C(O)—NH—; and L⁷ is independently substituted or unsubstituted alkylene.

In embodiments, -L³-L⁴- is independently —OPO₂—O-L⁷-NH—C(O)— or —OPO₂—O-L⁷-C(O)—NH—; and L⁷ is independently substituted or unsubstituted alkylene (e.g., C₁-C₂₀, C₁-C₁₂, C₁-C₈, C₁-C₆, C₁-C₄, or C₁-C₂). In embodiments, -L³-L⁴-is independently —OPO₂—O-L⁷-NH—C(O)—; and L⁷ is independently substituted or unsubstituted alkylene (e.g., C₁-C₂₀, C₁-C₁₂, C₁-C₈, C₁-C₆, C₁-C₄, or C₁-C₂). In embodiments, -L³-L⁴- is independently —OPO₂—O-L⁷-C(O)—NH—; and L⁷ is independently substituted or unsubstituted alkylene (e.g., C₁-C₂₀, C₁-C₁₂, C₁-C₈, C₁-C₆, C₁-C₄, or C₁-C₂).

In embodiments, -L³-L⁴- is independently —OP(O)(S)—O-L⁷-NH—C(O)— or —OP(O)(S)—O-L⁷-C(O)—NH—; and L⁷ is independently substituted or unsubstituted alkylene (e.g., C₁-C₂₀, C₁-C₁₂, C₁-C₈, C₁-C₆, C₁-C₄, or C₁-C₂). In embodiments, -L³-L⁴- is independently —OP(O)(S)—O-L⁷-NH—C(O)—; and L⁷ is independently substituted or unsubstituted alkylene (e.g., C₁-C₂₀, C₁-C₁₂, C₁-C₈, C₁-C₆, C₁-C₄, or C₁-C₂). In embodiments, -L³-L⁴- is independently —OP(O)(S)—O-L⁷-C(O)—NH—; and L⁷ is independently substituted or unsubstituted alkylene (e.g., C₁-C₂₀, C₁-C₁₂, C₁-C₈, C₁-C₆, C₁-C₄, or C₁-C₂).

In embodiments, -L³-L⁴- is independently —OPO₂—O-L⁷-C(O)—NH—; and L⁷ is independently substituted or unsubstituted C₁-C₈ alkylene. In embodiments, -L³-L⁴- is independently —OPO₂—O-L⁷-C(O)—NH—; and L⁷ is independently substituted C₁-C₈ alkylene. In embodiments, -L³-L⁴- is independently —OPO₂—O-L⁷-C(O)—NH—; and L⁷ is independently hydroxy(OH)-substituted C₁-C₈ alkylene. In embodiments, -L³-L⁴- is

independently —OPO₂—O-L⁷-C(O)—NH—; and L⁷ is independently hydroxymethyl-substituted C₁-C₈ alkylene. In embodiments, -L³-L⁴- is independently —OPO₂—O-L⁷-C(O)—NH—; and L⁷ is independently unsubstituted C₁-C₈ alkylene.

In embodiments, -L³-L⁴- is independently —OP(O)(S)—O-L⁷-C(O)—NH—; and L⁷ is independently substituted or unsubstituted C₁-C₈ alkylene. In embodiments, -L³-L⁴- is independently —OP(O)(S)—O-L⁷-C(O)—NH—; and L⁷ is independently substituted C₁-C₈ alkylene. In embodiments, -L³-L⁴- is independently —OP(O)(S)—O-L⁷-C(O)—NH—; and L⁷ is independently hydroxy(OH)-substituted C₁-C₈ alkylene. In embodiments, -L³-L⁴- is

independently —OP(O)(S)—O-L⁷-C(O)—NH—; and L⁷ is independently hydroxymethyl-substituted C₁-C₈ alkylene. In embodiments, -L³-L⁴- is independently —OP(O)(S)—O-L⁷-C(O)—NH—; and L⁷ is independently unsubstituted C₁-C₈ alkylene.

In embodiments, -L³-L⁴- is independently —OPO₂—O-L⁷-C(O)—NH—; and L⁷ is independently substituted or unsubstituted C₃-C₈ alkylene. In embodiments, -L³-L⁴- is independently —OPO₂—O-L⁷-C(O)—NH—; and L⁷ is independently substituted C₃-C₈ alkylene. In embodiments, -L³-L⁴- is independently —OPO₂—O-L⁷-C(O)—NH—; and L⁷ is independently hydroxy(OH)-substituted C₃-C₈ alkylene. In embodiments, -L³-L⁴- is

independently —OPO₂—O-L⁷-C(O)—NH—; and L⁷ is independently hydroxymethyl-substituted C₃-C₈ alkylene. In embodiments, -L³-L⁴- is independently —OPO₂—O-L⁷-C(O)—NH—; and L⁷ is independently unsubstituted C₃-C₈ alkylene.

In embodiments, -L³-L⁴- is independently —OP(O)(S)—O-L⁷-C(O)—NH—; and L⁷ is independently substituted or unsubstituted C₃-C₈ alkylene. In embodiments, -L³-L⁴- is independently —OP(O)(S)—O-L⁷-C(O)—NH—; and L⁷ is independently substituted C₃-C₈ alkylene. In embodiments, -L³-L⁴- is independently —OP(O)(S)—O-L⁷-C(O)—NH—; and L⁷ is independently hydroxy(OH)-substituted C₃-C₈ alkylene. In embodiments, -L³-L⁴- is

independently —OP(O)(S)—O-L⁷-C(O)—NH—; and L⁷ is independently hydroxymethyl-substituted C₃-C₈ alkylene. In embodiments, -L³-L⁴- is independently —OP(O)(S)—O-L⁷-C(O)—NH—; and L⁷ is independently unsubstituted C₃-C₈ alkylene.

In embodiments, -L³-L⁴- is independently —OPO₂—O-L⁷-C(O)—NH—; and L⁷ is independently substituted or unsubstituted C₅-C₈ alkylene. In embodiments, -L³-L⁴- is independently —OPO₂—O-L⁷-C(O)—NH—; and L⁷ is independently substituted C₅-C₈ alkylene. In embodiments, -L³-L⁴- is independently —OPO₂—O-L⁷-C(O)—NH—; and L⁷ is independently hydroxy(OH)-substituted C₅-C₈ alkylene. In embodiments, -L³-L⁴- is

independently —OPO₂—O-L⁷-C(O)—NH—; and L⁷ is independently hydroxymethyl-substituted C₅-C₈ alkylene. In embodiments, -L³-L⁴- is independently —OPO₂—O-L⁷-C(O)—NH—; and L⁷ is independently unsubstituted C₅-C₈ alkylene.

In embodiments, -L³-L⁴- is independently —OP(O)(S)—O-L⁷-C(O)—NH—; and L⁷ is independently substituted or unsubstituted C₅-C₈ alkylene. In embodiments, -L³-L⁴- is independently —OP(O)(S)—O-L⁷-C(O)—NH—; and L⁷ is independently substituted C₅-C₈alkylene. In embodiments, -L³-L⁴- is independently —OP(O)(S)—O-L⁷-C(O)—NH—; and L⁷ is independently hydroxy(OH)-substituted C₅-C₈ alkylene. In embodiments, -L³-L⁴- is

independently —OP(O)(S)—O-L⁷-C(O)—NH—; and L⁷ is independently hydroxymethyl-substituted C₅-C₈ alkylene. In embodiments, -L³-L⁴- is independently —OP(O)(S)—O-L⁷-C(O)—NH—; and L⁷ is independently unsubstituted C₅-C₈ alkylene.

In embodiments, -L³-L⁴- is independently —OPO₂—O-L⁷-NH—C(O)—; and L⁷ is independently substituted or unsubstituted C₁-C₈ alkylene. In embodiments, -L³-L⁴- is independently —OPO₂—O-L⁷-NH—C(O)—; and L⁷ is independently substituted C₁-C₈ alkylene. In embodiments, -L³-L⁴- is independently —OPO₂—O-L⁷-NH—C(O)—; and L⁷ is independently hydroxy(OH)-substituted C₁-C₈ alkylene. In embodiments, -L³-L⁴- is

independently —OPO₂—O-L⁷-NH—C(O)—; and L⁷ is independently hydroxymethyl-substituted C₁-C₈ alkylene. In embodiments, -L³-L⁴- is independently —OPO₂—O-L⁷-NH—C(O)—; and L⁷ is independently unsubstituted C₁-C₈ alkylene.

In embodiments, -L³-L⁴- is independently —OP(O)(S)—O-L⁷-NH—C(O)—; and L⁷ is independently substituted or unsubstituted C₁-C₈ alkylene. In embodiments, -L³-L⁴- is independently —OP(O)(S)—O-L⁷-NH—C(O)—; and L⁷ is independently substituted C₁-C₈ alkylene. In embodiments, -L³-L⁴- is independently —OP(O)(S)₂—O-L⁷-NH—C(O)—; and L⁷ is independently hydroxy(OH)-substituted C₁-C₈ alkylene. In embodiments, -L³-L⁴- is

independently —OP(O)(S)—O-L⁷-NH—C(O)—; and L⁷ is independently hydroxymethyl-substituted C₁-C₈ alkylene. In embodiments, -L³-L⁴- is independently —OP(O)(S)—O-L⁷-NH—C(O)—; and L⁷ is independently unsubstituted C₁-C₈ alkylene.

In embodiments, -L³-L⁴- is independently —OPO₂—O-L⁷-NH—C(O)—; and L⁷ is independently substituted or unsubstituted C₃-C₈ alkylene. In embodiments, -L³-L⁴- is independently —OPO₂—O-L⁷-NH—C(O)—; and L⁷ is independently substituted C₃-C₈ alkylene. In embodiments, -L³-L⁴- is independently —OPO₂—O-L⁷-NH—C(O)—; and L⁷ is independently hydroxy(OH)-substituted C₃-C₈ alkylene. In embodiments, -L³-L⁴- is

independently —OPO₂—O-L⁷-NH—C(O)—; and L⁷ is independently hydroxymethyl-substituted C₃-C₈ alkylene. In embodiments, -L³-L⁴- is independently —OPO₂—O-L⁷-NH—C(O)—; and L⁷ is independently unsubstituted C₃-C₈ alkylene.

In embodiments, -L³-L⁴- is independently —OP(O)(S)—O-L⁷-NH—C(O)—; and L⁷ is independently substituted or unsubstituted C₃-C₈ alkylene. In embodiments, -L³-L⁴- is independently —OP(O)(S)—O-L⁷-NH—C(O)—; and L⁷ is independently substituted C₃-C₈ alkylene. In embodiments, -L³-L⁴- is independently —OP(O)(S)—O-L⁷-NH—C(O)—; and L⁷ is independently hydroxy(OH)-substituted C₃-C₈ alkylene. In embodiments, -L³-L⁴- is

independently —OP(O)(S)—O-L⁷-NH—C(O)—; and L⁷ is independently hydroxymethyl-substituted C₃-C₈ alkylene. In embodiments, -L³-L⁴- is independently —OP(O)(S)—O-L⁷-NH—C(O)—; and L⁷ is independently unsubstituted C₃-C₈ alkylene.

In embodiments, -L³-L⁴- is independently —OPO₂—O-L⁷-NH—C(O)—; and L⁷ is independently substituted or unsubstituted C₅-C₈ alkylene. In embodiments, -L³-L⁴- is independently —OPO₂—O-L⁷-NH—C(O)—; and L⁷ is independently substituted C₅-C₈ alkylene. In embodiments, -L³-L⁴- is independently —OPO₂—O-L⁷-NH—C(O)—; and L⁷ is independently hydroxy(OH)-substituted C₅-C₈ alkylene. In embodiments, -L³-L⁴- is

independently —OPO₂—O-L⁷-NH—C(O)—; and L⁷ is independently hydroxymethyl-substituted C₅-C₈ alkylene. In embodiments, -L³-L⁴- is independently —OPO₂—O-L⁷-NH—C(O)—; and L⁷ is independently unsubstituted C₅-C₈ alkylene.

In embodiments, -L³-L⁴- is independently —OP(O)(S)—O-L⁷-NH—C(O)—; and L⁷ is independently substituted or unsubstituted C₅-C₈ alkylene. In embodiments, -L³-L⁴- is independently —OP(O)(S)—O-L⁷-NH—C(O)—; and L⁷ is independently substituted C₅-C₈ alkylene. In embodiments, -L³-L⁴- is independently —OP(O)(S)—O-L⁷-NH—C(O)—; and L⁷ is independently hydroxy(OH)-substituted C₅-C₈ alkylene. In embodiments, -L³-L⁴- is

independently —OP(O)(S)—O-L⁷-NH—C(O)—; and L⁷ is independently hydroxymethyl-substituted C₅-C₈ alkylene. In embodiments, -L³-L⁴- is independently —OP(O)(S)—O-L⁷-NH—C(O)—; and L⁷ is independently unsubstituted C₅-C₈ alkylene.

In embodiments, -L³-L⁴- is attached to a 3′ carbon of the oligonucleotide. In embodiments, -L³-L⁴- is attached to a 3′ nitrogen of the oligonucleotide (e.g., the 3′ nitrogen of a morpholino moiety). In embodiments, -L³-L⁴- is attached to a 5′ carbon of the oligonucleotide. In embodiments, -L³-L⁴- is attached to a 6′ carbon of the oligonucleotide (e.g., the 6′ carbon of a morpholino moiety). In embodiments, -L³-L⁴- is attached to a 2′ carbon of the oligonucleotide. In embodiments, -L³-L⁴- is attached to a nucleobase of the oligonucleotide.

In embodiments, at least -L³-L⁴- is attached to a 3′ carbon of the oligonucleotide at a 3′ end. In embodiments, at least -L³-L⁴- is attached to a 3′ nitrogen of the oligonucleotide (e.g., the 3′ nitrogen of a morpholino moiety) at a 3′ end. In embodiments, at least -L³-L⁴- is attached to a 5′ carbon of the oligonucleotide at a 5′ end. In embodiments, at least -L³-L⁴- is attached to a 6′ carbon of the oligonucleotide (e.g., the 6′ carbon of a morpholino moiety) at a 5′ end.

In embodiments, -L³-L⁴- is independently

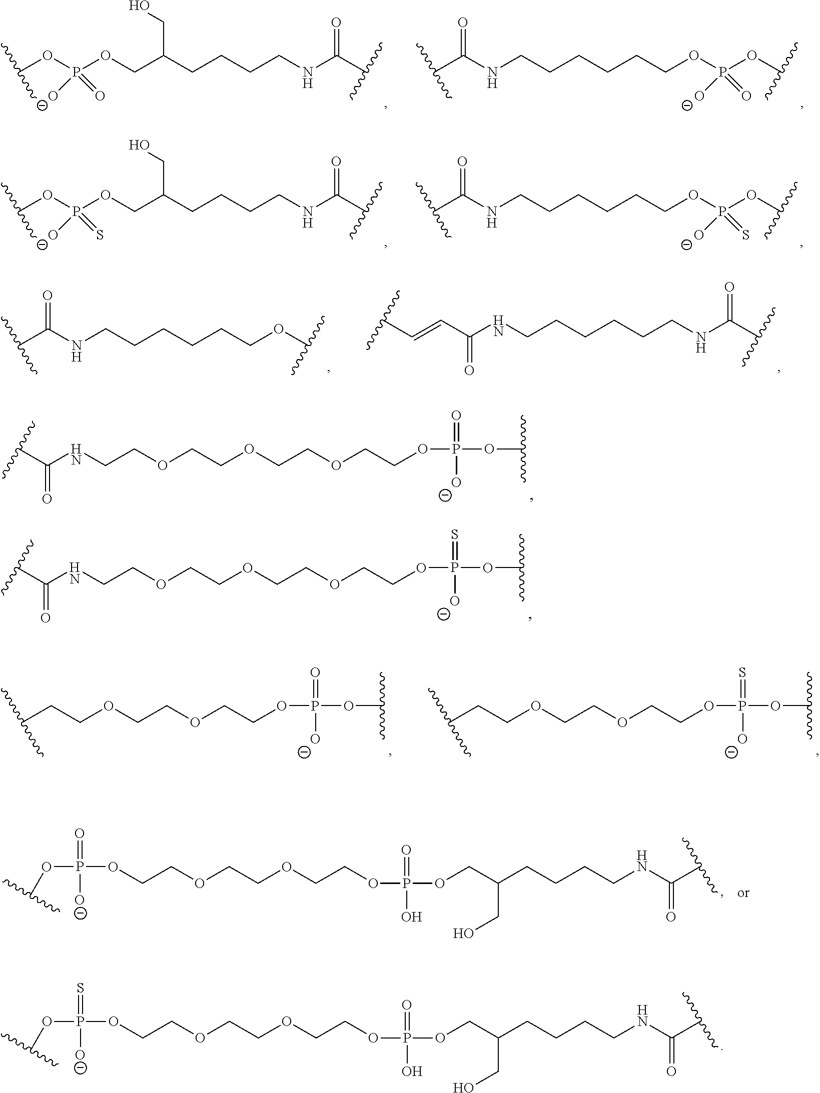

In embodiments, -L³-L⁴- is independently

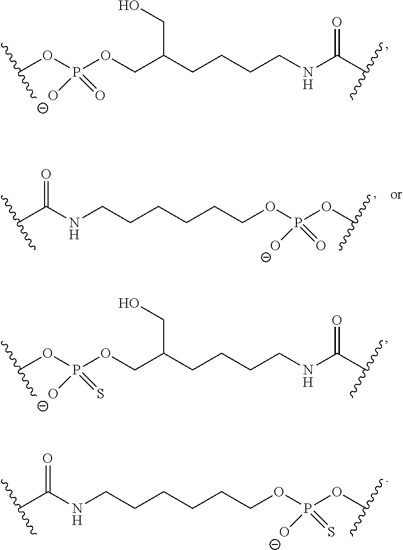 [SMILES: *OP(=O)([O-])OCC(CO)CCCCNC(*)=O ; *OP(=O)([O-])OCCCCCCNC(*)=O ; *OP([O-])(=S)OCC(CO)CCCCNC(*)=O ; *OP([O-])(=S)OCCCCCCNC(*)=O]

In embodiments, -L³-L⁴- is independently

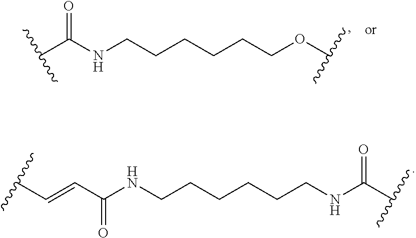 [SMILES: *OCCCCCCNC(*)=O ; */C=C/C(=O)NCCCCCCNC(*)=O]

In embodiments, -L³-L⁴- is independently

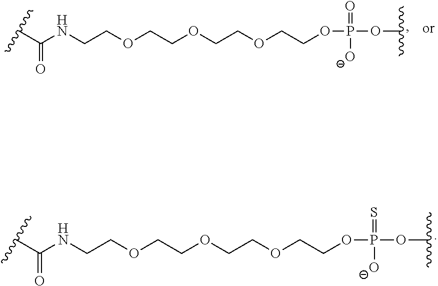 [SMILES: *OP(=O)([O-])OCCOCCOCCOCCNC(*)=O ; *OP([O-])(=S)OCCOCCOCCOCCNC(*)=O]

In embodiments, -L³-L⁴- is independently

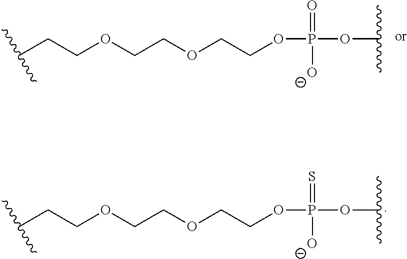 [SMILES: *CCOCCOCCOP(=O)([O-])O* ; *CCOCCOCCOP([O-])(=S)O*]

In embodiments, -L³-L⁴- is independently

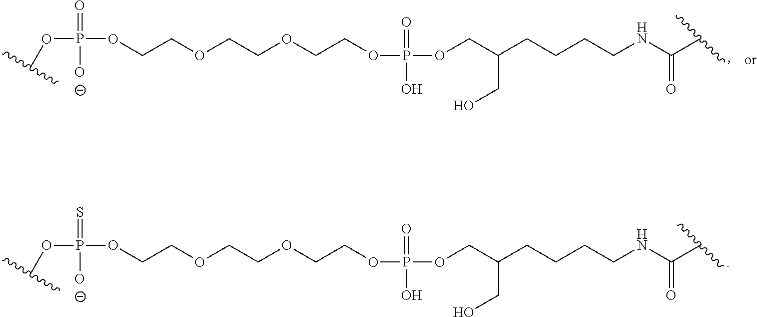 [SMILES: *OP(=O)([O-])OCCOCCOCCOP(=O)(O)OCC(CO)CCCCNC(*)=O ; *OP([O-])(=S)OCCOCCOCCOP(=O)(O)OCC(CO)CCCCNC(*)=O]

In embodiments, -L³-L⁴- is independently

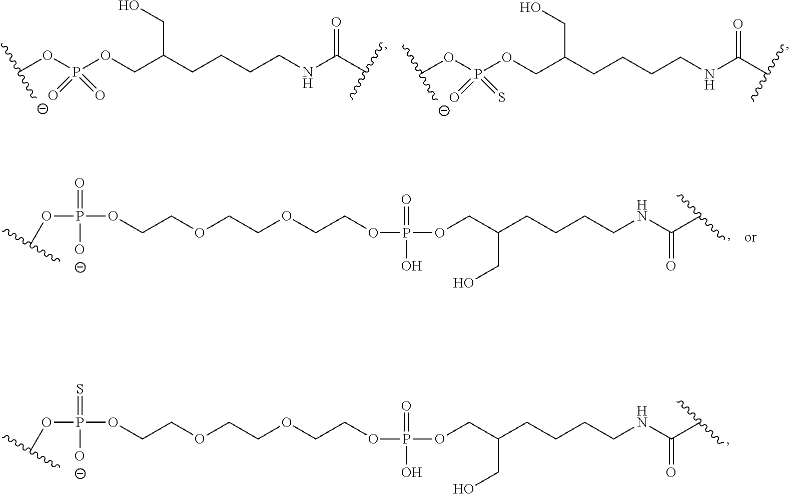

and is attached to a 3′ carbon of the oligonucleotide.
In embodiments, -L³-L⁴- is independently

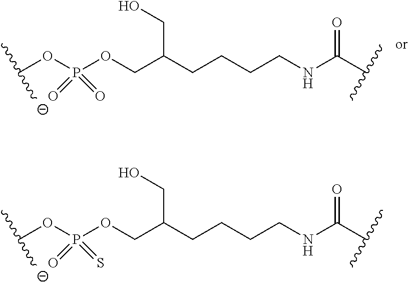

that is attached to a 3′ carbon of the oligonucleotide. In embodiments, -L³-L⁴- is independently

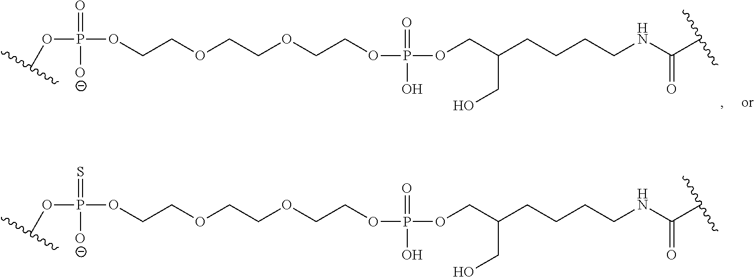 [SMILES: *OP(=O)([O-])OCCOCCOCCOP(=O)(O)OCC(CO)CCCCNC(*)=O ; *OP([O-])(=S)OCCOCCOCCOP(=O)(O)OCC(CO)CCCCNC(*)=O]

that is attached to a 3′ carbon of the oligonucleotide.

In embodiments, -L³-L⁴- is independently

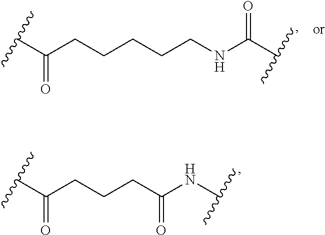 [SMILES: *C(=O)CCCCCNC(*)=O ; *NC(=O)CCCC(*)=O]

and is attached to a 3′ nitrogen of the oligonucleotide (e.g., the 3′ nitrogen of a morpholino moiety).
In embodiments, -L³-L⁴- is independently

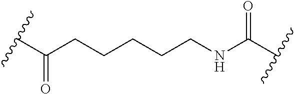 [SMILES: *C(=O)CCCCCNC(*)=O]

attached to a 3′ nitrogen of the oligonucleotide (e.g., the 3′ nitrogen of a morpholino moiety). In embodiments, -L³-L⁴- is independently

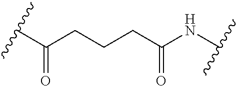 [SMILES: *NC(=O)CCCC(*)=O]

that is attached to a 3′ nitrogen of the oligonucleotide (e.g., the 3′ nitrogen of a morpholino moiety).

In embodiments, an -L³-L⁴- is independently

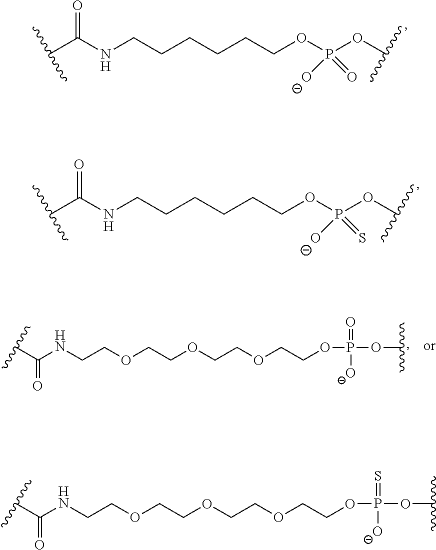 [SMILES: *OP(=O)([O-])OCCCCCCNC(*)=O ; *OP([O-])(=S)OCCCCCCNC(*)=O ; *OP(=O)([O-])OCCOCCOCCOCCNC(*)=O ; *OP([O-])(=S)OCCOCCOCCOCCNC(*)=O]

and is attached to a 5′ carbon of the oligonucleotide. In embodiments, an -L³-L⁴- is independently

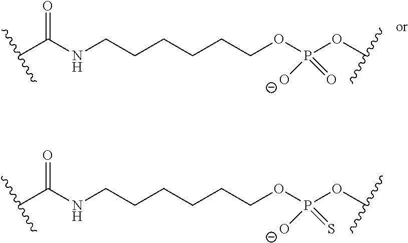 [SMILES: *OP(=O)([O-])OCCCCCCNC(*)=O ; *OP([O-])(=S)OCCCCCCNC(*)=O]

that is attached to a 5′ carbon of the oligonucleotide. In embodiments, an -L³-L⁴- is independently

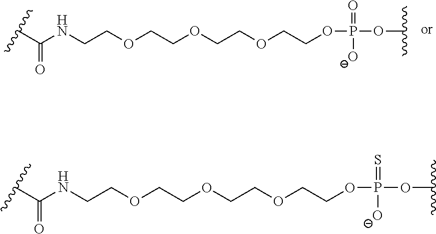 [SMILES: *OP(=O)([O-])OCCOCCOCCOCCNC(*)=O ; *OP([O-])(=S)OCCOCCOCCOCCNC(*)=O]

that is attached to a 5′ carbon of the oligonucleotide.

In embodiments where the oligonucleotide includes a morpholino moiety, L³ is independently —P(O)(N(CH₃)₂)—N— or —P(O)(N(CH₃)₂)—O—. In embodiments, L⁴ is substituted or unsubstituted heterocycloalkyl. In embodiments, L⁴ is substituted heterocycloalkyl. In embodiments, L⁴ is unsubstituted heterocycloalkyl. In embodiments, L⁴ is substituted or unsubstituted piperidinylene. In embodiments, L⁴ is substituted piperidinylene. In embodiments, L⁴ is unsubstituted piperidinylene. In embodiments, L⁴ is substituted or unsubstituted piperazinylene. In embodiments, L⁴ is substituted piperazinylene. In embodiments, L⁴ is unsubstituted piperazinylene. In embodiments, an -L³-L⁴- is independently

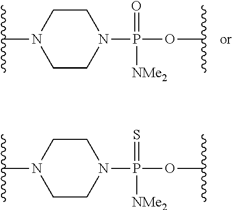 [SMILES: *OP(=O)(N(C)C)N1CCN(*)CC1 ; *OP(=S)(N(C)C)N1CCN(*)CC1]

is attached to a 6′ carbon of the oligonucleotide (e.g., the 6′ carbon of a morpholino moiety). In embodiments, an -L³-L⁴- is independently

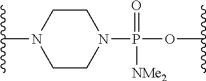 [SMILES: *OP(=O)(N(C)C)N1CCN(*)CC1]

that is attached to a 6′ carbon of the oligonucleotide (e.g., the 6′ carbon of a morpholino moiety). In embodiments, an -L³-L⁴- is independently

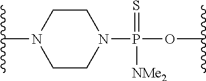 [SMILES: *OP(=S)(N(C)C)N1CCN(*)CC1]

that is attached to a 6′ carbon of the oligonucleotide (e.g., the 6′ carbon of a morpholino moiety).

In embodiments, an -L³-L⁴- is independently is attached to a nucleobase of the oligonucleotide. In embodiments, an -L³-L⁴- is independently

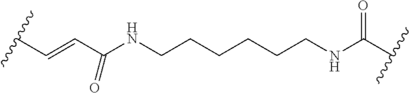 [SMILES: */C=C/C(=O)NCCCCCCNC(*)=O]

and is attached to a nucleobase of the oligonucleotide.

In embodiments, -L³-L⁴- is attached to a 3′ carbon of the double-stranded oligonucleotide at either of its 3′ ends. In embodiments, -L³-L⁴- is attached to a 3′ carbon of the double-stranded oligonucleotide at the 3′end of its antisense strand. In embodiments, -L³-L⁴- is attached to a 3′ carbon of the double-stranded oligonucleotide at the 3′end of its sense strand

In embodiments, -L³-L⁴- is attached to a 3′ nitrogen of the double-stranded oligonucleotide (e.g., the 3′ nitrogen of a morpholino moiety) at either of its 3′ ends. In embodiments, -L³-L⁴- is attached to a 3′ nitrogen of the double-stranded oligonucleotide (e.g., the 3′ nitrogen of a morpholino moiety) at the 3′end of its antisense strand. In embodiments, -L³-L⁴- is attached to a 3′ nitrogen of the double-stranded oligonucleotide (e.g., PMO) at the 3′end of its sense strand.

In embodiments, -L³-L⁴- is attached to a 5′ carbon of the double-stranded oligonucleotide at either of its 5′ ends. In embodiments, -L³-L⁴- is attached to a 5′ carbon of the double-stranded oligonucleotide at the 5′ end of its antisense strand. In embodiments, -L³-L⁴- is attached to a 5′ carbon of the double-stranded oligonucleotide at the 5′ end of its sense strand.

In embodiments, -L³-L⁴- is attached to a 6′ carbon of the double-stranded oligonucleotide (e.g., the 6′ carbon of the morpholino moiety) at either of its 5′ ends. In embodiments, -L³-L⁴- is attached to a 6′ carbon of the double-stranded oligonucleotide (e.g., the 6′ carbon of the morpholino moiety) at the 5′ end of its antisense strand. In embodiments, -L³-L⁴- is attached to a 6′ carbon of the double-stranded oligonucleotide (e.g., the 6′ carbon of the morpholino moiety) at the 5′ end of its sense strand.

In embodiments, -L³-L⁴- is attached to a 2′ carbon of the double-stranded oligonucleotide. In embodiments, -L³-L⁴- is attached to a 2′ carbon of the double-stranded oligonucleotide at either of its 3′ ends. In embodiments, -L³-L⁴- is attached to a 2′ carbon of the double-stranded oligonucleotide at the 3′end of its antisense strand. In embodiments, -L³-L⁴- is attached to a 2′ carbon of the double-stranded oligonucleotide at the 3′end of its sense strand. In embodiments, -L³-L⁴- is attached to a 2′ carbon of the double-stranded oligonucleotide at either of its 5′ ends. In embodiments, -L³-L⁴- is attached to a 2′ carbon of the double-stranded oligonucleotide at the 5′ end of its antisense strand. In embodiments, -L³-L⁴- is attached to a 2′ carbon of the double-stranded oligonucleotide at the 5′ end of its sense strand.

In embodiments, -L³-L⁴- is attached to a nucleobase of the double-stranded oligonucleotide. In embodiments, -L³-L⁴- is attached to a nucleobase of the double-stranded oligonucleotide at either of its 3′ ends. In embodiments, -L³-L⁴- is attached to a nucleobase of the double-stranded oligonucleotide at the 3′end of its antisense strand. In embodiments, -L³-L⁴- is attached to a nucleobase of the double-stranded oligonucleotide at the 3′end of its sense strand. In embodiments, -L³-L⁴- is attached to a nucleobase of the double-stranded oligonucleotide at either of its 5′ ends. In embodiments, -L³-L⁴- is attached to a nucleobase of the double-stranded oligonucleotide at the 5′ end of its antisense strand. In embodiments, -L³-L⁴- is attached to a nucleobase of the double-stranded oligonucleotide at the 5′ end of its sense strand.

In embodiments, -L³-L⁴- is attached to a 3′ carbon of the single-stranded oligonucleotide at the 3′ end.

In embodiments, -L³-L⁴- is attached to a 3′ nitrogen of the single-stranded oligonucleotide at the 3′ end (e.g., the 3′ nitrogen of a morpholino moiety).

In embodiments, -L³-L⁴- is attached to a 5′ carbon of the single-stranded oligonucleotide at the 5′ end of the single-stranded oligonucleotide.

In embodiments, -L³-L⁴- is attached to a 6′ carbon of the single-stranded oligonucleotide at its 5′ end (e.g., the 6′ carbon of a morpholino moiety). In embodiments, -L³-L⁴- is independently

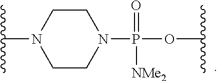 [SMILES: *OP(=O)(N(C)C)N1CCN(*)CC1]

In embodiments, -L³-L⁴- is attached to a 2′ carbon of the single-stranded oligonucleotide. In embodiments, -L³-L⁴- is attached to a 2′ carbon of the single-stranded oligonucleotide at the 3′ end. In embodiments, -L³-L⁴- is attached to a 2′ carbon of the single-stranded oligonucleotide at the 5′ end.

In embodiments, -L³-L⁴- is attached to a nucleobase of the single-stranded oligonucleotide. In embodiments, -L³-L⁴- is attached to a nucleobase of the single-stranded oligonucleotide at its of 3′ end. In embodiments, -L³-L⁴- is attached to a nucleobase of the single-stranded oligonucleotide at its 5′ end.

In embodiments, -L³-L⁴- is independently

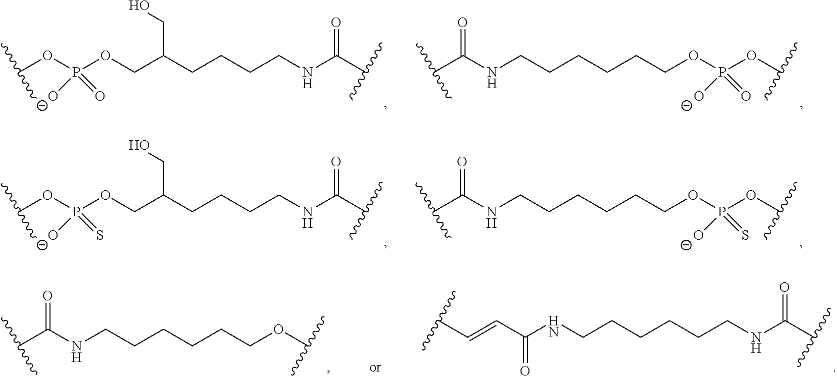 [SMILES: *OP(=O)([O-])OCC(CO)CCCCNC(*)=O ; *OP(=O)([O-])OCCCCCCNC(*)=O ; *OP([O-])(=S)OCC(CO)CCCCNC(*)=O ; *OP([O-])(=S)OCCCCCCNC(*)=O ; *OCCCCCCNC(*)=O ; */C=C/C(=O)NCCCCCCNC(*)=O]

In embodiments, -L³-L⁴- is independently

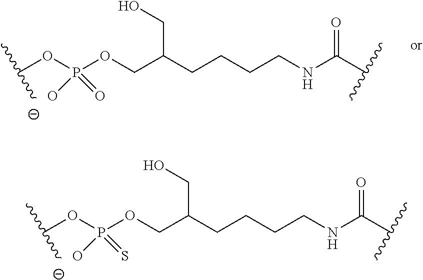 [SMILES: *OP(=O)(O)OCC(CO)CCCCNC(*)=O ; *OP(O)(=S)OCC(CO)CCCCNC(*)=O]

that is attached to a 3′ carbon of the oligonucleotide.
In embodiments -L³-L⁴- is independently

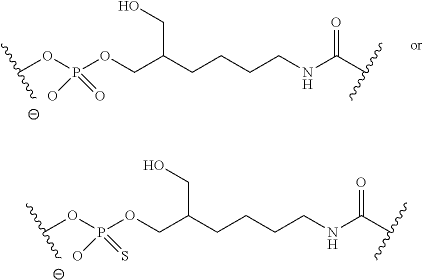 [SMILES: *OP(=O)(O)OCC(CO)CCCCNC(*)=O ; *OP(O)(=S)OCC(CO)CCCCNC(*)=O]

that is attached to a 3′ nitrogen of the oligonucleotide (e.g., the 3′ nitrogen of a morpholino moiety). In embodiments, -L³-L⁴- is independently

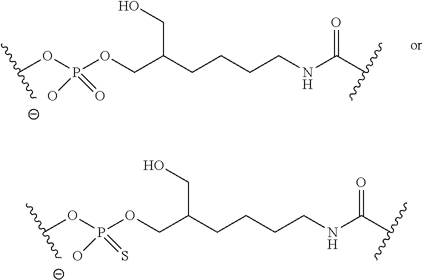 [SMILES: *OP(=O)(O)OCC(CO)CCCCNC(*)=O ; *OP(O)(=S)OCC(CO)CCCCNC(*)=O]

that is attached to a 5′ carbon of the oligonucleotide. In embodiments, -L³-L⁴- is independently

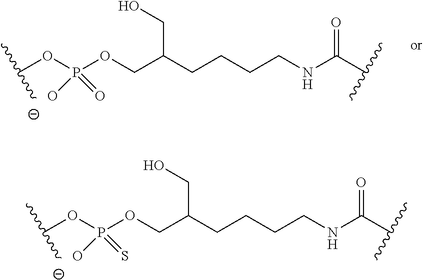 [SMILES: *OP(=O)(O)OCC(CO)CCCCNC(*)=O ; *OP(O)(=S)OCC(CO)CCCCNC(*)=O]

that is attached to a 6′ carbon of the oligonucleotide (e.g., the 6′ carbon of a morpholino moiety). In embodiments, -L³-L⁴- is independently

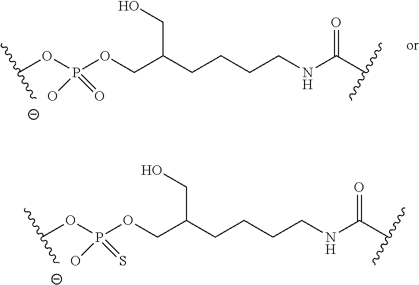 [SMILES: *OP(=O)(O)OCC(CO)CCCCNC(*)=O ; *OP(O)(=S)OCC(CO)CCCCNC(*)=O]

that is attached to a 2′ carbon of the oligonucleotide. In embodiments, -L³-L⁴- is independently

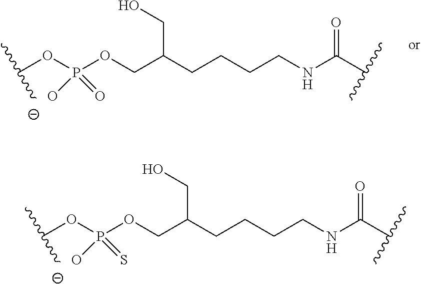 [SMILES: *OP(=O)(O)OCC(CO)CCCCNC(*)=O ; *OP(O)(=S)OCC(CO)CCCCNC(*)=O]

that is attached to a nucleobase of the oligonucleotide.

In embodiments, -L³-L⁴- is independently

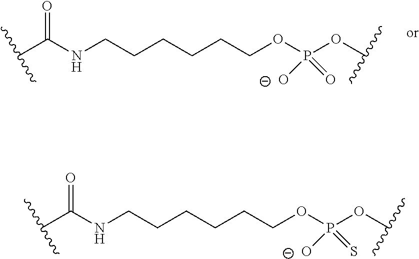 [SMILES: *OP(=O)([O-])OCCCCCCNC(*)=O ; *OP([O-])(=S)OCCCCCCNC(*)=O]

that is attached to a 3′ carbon of the oligonucleotide. In embodiments, -L³-L⁴- is independently

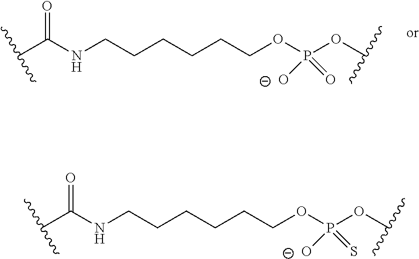 [SMILES: *OP(=O)([O-])OCCCCCCNC(*)=O ; *OP([O-])(=S)OCCCCCCNC(*)=O]

that is attached to a 3′ nitrogen of the oligonucleotide (e.g., the 3′ nitrogen of a morpholino moiety). In embodiments, -L³-L⁴- is independently

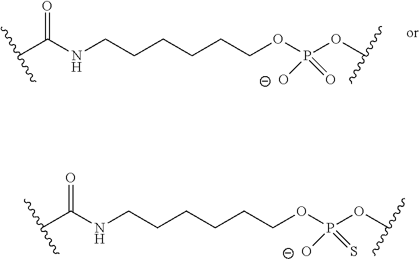 [SMILES: *OP(=O)([O-])OCCCCCCNC(*)=O ; *OP([O-])(=S)OCCCCCCNC(*)=O]

that is attached to a 5′ carbon of the oligonucleotide. In embodiments, -L³-L⁴- is independently

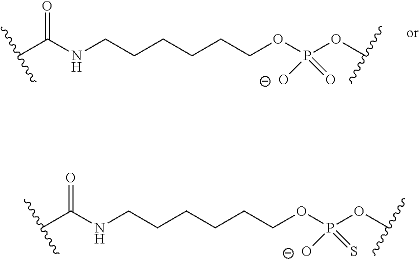 [SMILES: *OP(=O)([O-])OCCCCCCNC(*)=O ; *OP([O-])(=S)OCCCCCCNC(*)=O]

that is attached to a 6′ carbon of the oligonucleotide (e.g., the 6′ carbon of a morpholino moiety MO). In embodiments, -L³-L⁴- is independently

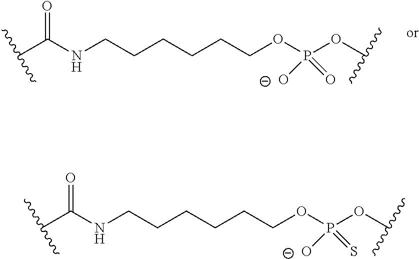 [SMILES: *OP(=O)([O-])OCCCCCCNC(*)=O ; *OP([O-])(=S)OCCCCCCNC(*)=O]

that is attached to a 2′ carbon of the oligonucleotide. In embodiments, -L³-L⁴- is independently

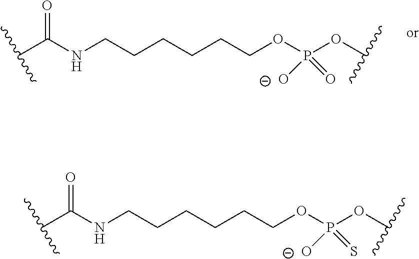 [SMILES: *OP(=O)([O-])OCCCCCCNC(*)=O ; *OP([O-])(=S)OCCCCCCNC(*)=O]

that is attached to a nucleobase of the oligonucleotide.

In embodiments, -L³-L⁴- is independently

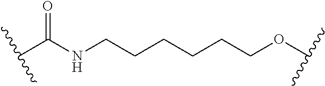 [SMILES: *OCCCCCCNC(*)=O]

that is attached to a 3′ carbon of the oligonucleotide. In embodiments, -L³-L⁴- is independently

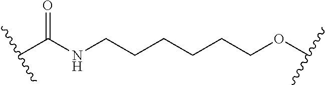 [SMILES: *OCCCCCCNC(*)=O]

that is attached to a 3′ nitrogen of the oligonucleotide (e.g., the 3′ nitrogen of a morpholino moiety). In embodiments, -L³-L⁴- is independently

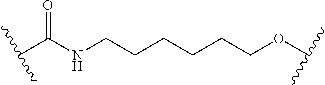 [SMILES: *OCCCCCCNC(*)=O]

and is attached to a 5′ carbon of the oligonucleotide. In embodiments, -L³-L⁴- is independently

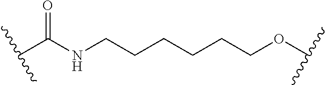 [SMILES: *OCCCCCCNC(*)=O]

that is attached to a 6′ carbon of the oligonucleotide (e.g., the 6′ carbon of a morpholino moiety). In embodiments, -L³-L⁴- is independently

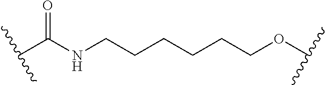 [SMILES: *OCCCCCCNC(*)=O]

and is attached to a 2′ carbon of the oligonucleotide. In embodiments, -L³-L⁴- is independently

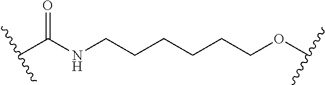 [SMILES: *OCCCCCCNC(*)=O]

and is attached to a nucleobase of the oligonucleotide.

In embodiments, -L³-L⁴- is independently

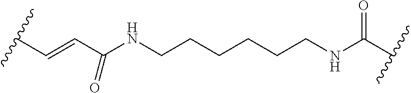 [SMILES: */C=C/C(=O)NCCCCCCNC(*)=O]

that is attached to a 3′ carbon of the oligonucleotide.
In embodiments, -L³-L⁴- is independently

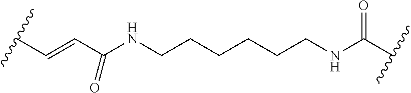 [SMILES: */C=C/C(=O)NCCCCCCNC(*)=O]

that is attached to a 3′ nitrogen of the oligonucleotide (e.g., the 3′ nitrogen of a morpholino moiety).
In embodiments, -L³-L⁴- is independently

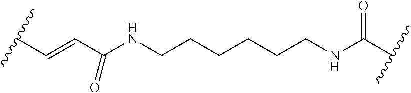 [SMILES: */C=C/C(=O)NCCCCCCNC(*)=O]

that is attached to a 5′ carbon of the oligonucleotide.
In embodiments, -L³-L⁴- is independently

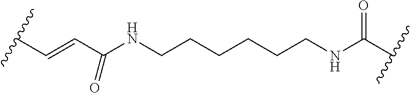 [SMILES: */C=C/C(=O)NCCCCCCNC(*)=O]

that is attached to a 6′ carbon of the oligonucleotide (e.g., the 6′ carbon of a morpholino moiety).
In embodiments, -L³-L⁴- is independently

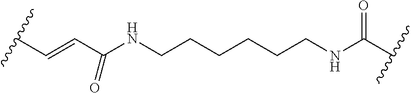 [SMILES: */C=C/C(=O)NCCCCCCNC(*)=O]

that is attached to a 2′ carbon of the oligonucleotide. In embodiments, -L³-L⁴- is independently

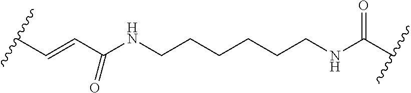 [SMILES: */C=C/C(=O)NCCCCCCNC(*)=O]

and is attached to a nucleobase of the oligonucleotide.

In embodiments, R³ is independently

hydrogen, —NH₂, —OH, —SH, —C(O)H, —C(O)NH₂, —NHC(O)H, —NHC(O)OH, —NHC(O)NH₂, —C(O) OH, —OC(O)H, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R³ is independently hydrogen. In embodiments, R³ is independently —NH₂. In embodiments, R³ is independently —OH. In embodiments, R³ is independently —SH. In embodiments, R³ is independently —C(O)H. In embodiments, R³ is independently —C(O)NH₂. In embodiments, R³ is independently —NHC(O)H. In embodiments, R³ is independently —NHC(O)OH. In embodiments, R³ is independently —NHC(O)NH₂. In embodiments, R³ is independently —C(O)OH. In embodiments, R³ is independently —OC(O)H. In embodiments, R³ is independently —N₃.

In embodiments, R³ is independently substituted or unsubstituted alkyl (e.g., C₁-C₂₀, C₁-C₁₂, C₁-C₈, C₁-C₆, C₁-C₄, or C₁-C₂). In embodiments, R³ is independently substituted or unsubstituted C₁-C₂₀ alkyl. In embodiments, R³ is independently substituted C₁-C₂₀ alkyl. In embodiments, R³ is independently unsubstituted C₁-C₂₀ alkyl. In embodiments, R³ is independently substituted or unsubstituted C₁-C₁₂ alkyl. In embodiments, R³ is independently substituted C₁-C₁₂ alkyl. In embodiments, R³ is independently unsubstituted C₁-C₁₂ alkyl. In embodiments, R³ is independently substituted or unsubstituted C₁-C₈ alkyl. In embodiments, R³ is independently substituted C₁-C₈ alkyl. In embodiments, R³ is independently unsubstituted C₁-C₈ alkyl. In embodiments, R³ is independently substituted or unsubstituted C₁-C₆ alkyl. In embodiments, R³ is independently substituted C₁-C₆ alkyl. In embodiments, R³ is independently unsubstituted C₁-C₆ alkyl. In embodiments, R³ is independently substituted or unsubstituted C₁-C₄ alkyl. In embodiments, R³ is independently substituted C₁-C₄ alkyl. In embodiments, R³ is independently unsubstituted C₁-C₄ alkyl. In embodiments, R³ is independently substituted or unsubstituted ethyl. In embodiments, R³ is independently substituted ethyl. In embodiments, R³ is independently unsubstituted ethyl. In embodiments, R³ is independently substituted or unsubstituted methyl. In embodiments, R³ is independently substituted methyl. In embodiments, R³ is independently unsubstituted methyl.

In embodiments, L⁶ is independently —NHC(O)—. In embodiments, L⁶ is independently —C(O)NH—. In embodiments, L⁶ is independently substituted or unsubstituted alkylene. In embodiments, L⁶ is independently substituted or unsubstituted heteroalkylene.

In embodiments, L⁶ is independently substituted or unsubstituted alkylene (e.g., C₁-C₂₀, C₁-C₁₂, C₁-C₈, C₁-C₆, C₁-C₄, or C₁-C₂). In embodiments, L⁶ is independently substituted alkylene (e.g., C₁-C₂₀, C₁-C₁₂, C₁-C₈, C₁-C₆, C₁-C₄, or C₁-C₂). In embodiments, L⁶ is independently unsubstituted alkylene (e.g., C₁-C₂₀, C₁-C₁₂, C₁-C₈, C₁-C₆, C₁-C₄, or C₁-C₂). In embodiments, L⁶ is independently substituted or unsubstituted C₁-C₂₀ alkylene. In embodiments, L⁶ is independently substituted C₁-C₂₀ alkylene. In embodiments, L⁶ is independently unsubstituted C₁-C₂₀ alkylene. In embodiments, L⁶ is independently substituted or unsubstituted C₁-C₁₂ alkylene. In embodiments, L⁶ is independently substituted C₁-C₁₂ alkylene. In embodiments, L⁶ is independently unsubstituted C₁-C₁₂ alkylene. In embodiments, L⁶ is independently substituted or unsubstituted C₁-C₈ alkylene. In embodiments, L⁶ is independently substituted C₁-C₈ alkylene. In embodiments, L⁶ is independently unsubstituted C₁-C₈ alkylene. In embodiments, L⁶ is independently substituted or unsubstituted C₁-C₆ alkylene. In embodiments, L⁶ is independently substituted C₁-C₆ alkylene. In embodiments, L⁶ is independently unsubstituted C₁-C₆ alkylene. In embodiments, L⁶ is independently substituted or unsubstituted C₁-C₄ alkylene. In embodiments, L⁶ is independently substituted C₁-C₄ alkylene. In embodiments, L⁶ is independently unsubstituted C₁-C₄ alkylene. In embodiments, L⁶ is independently substituted or unsubstituted ethylene. In embodiments, L⁶ is independently substituted ethylene. In embodiments, L⁶ is independently unsubstituted ethylene. In embodiments, L⁶ is independently substituted or unsubstituted methylene. In embodiments, L⁶ is independently substituted methylene. In embodiments, L⁶ is independently unsubstituted methylene.

In embodiments, L⁶ is independently substituted or unsubstituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L⁶ is independently substituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L⁶ is independently unsubstituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L⁶ is independently substituted or unsubstituted 2 to 20 membered heteroalkylene. In embodiments, L⁶ is independently substituted 2 to 20 membered heteroalkylene. In embodiments, L⁶ is independently unsubstituted 2 to 20 membered heteroalkylene. In embodiments, L⁶ is independently substituted or unsubstituted 2 to 8 membered heteroalkylene. In embodiments, L⁶ is independently substituted 2 to 8 membered heteroalkylene. In embodiments, L⁶ is independently unsubstituted 2 to 8 membered heteroalkylene. In embodiments, L⁶ is independently substituted or unsubstituted 2 to 6 membered heteroalkylene. In embodiments, L⁶ is independently substituted 2 to 6 membered heteroalkylene. In embodiments, L⁶ is independently unsubstituted 2 to 6 membered heteroalkylene. In embodiments, L⁶ is independently substituted or unsubstituted 4 to 6 membered heteroalkylene. In embodiments, L⁶ is independently substituted 4 to 6 membered heteroalkylene. In embodiments, L⁶ is independently unsubstituted 4 to 6 membered heteroalkylene. In embodiments, L⁶ is independently substituted or unsubstituted 2 to 3 membered heteroalkylene. In embodiments, L⁶ is independently substituted 2 to 3 membered heteroalkylene. In embodiments, L⁶ is independently unsubstituted 2 to 3 membered heteroalkylene. In embodiments, L⁶ is independently substituted or unsubstituted 4 to 5 membered heteroalkylene. In embodiments, L⁶ is independently substituted 4 to 5 membered heteroalkylene. In embodiments, L⁶ is independently unsubstituted 4 to 5 membered heteroalkylene.

In embodiments, L^{6A} is independently a bond or unsubstituted alkylene; L^{6B} is independently a bond, —NHC(O)—, or unsubstituted arylene; L^{6C} is independently a bond, unsubstituted alkylene, or unsubstituted arylene; L^{6D} is independently a bond or unsubstituted alkylene; and L^{6E} is independently a bond or —NHC(O)—. In embodiments, L^{6A} is independently a bond or unsubstituted alkylene. In embodiments, L^{6B} is independently a bond, —NHC(O)—, or unsubstituted arylene. In embodiments, L^{6C} is independently a bond, unsubstituted alkylene, or unsubstituted arylene. In embodiments, L^{6D} is independently a bond or unsubstituted alkylene. In embodiments, L^{6E} is independently a bond or —NHC(O)—.

In embodiments, L^{6A} is independently a bond or unsubstituted alkylene (e.g., C₁-C₂₀, C₁-C₁₂, C₁-C₈, C₁-C₆, C₁-C₄, or C₁-C₂). In embodiments, L^{6A} is independently unsubstituted C₁-C₂₀ alkylene. In embodiments, L^{6A} is independently unsubstituted C₁-C₁₂ alkylene. In embodiments, L^{6A} is independently unsubstituted C₁-C₈ alkylene. In embodiments, L^{6A} is independently unsubstituted C₁-C₆ alkylene. In embodiments, L^{6A} is independently unsubstituted C₁-C₄ alkylene. In embodiments, L^{6A} is independently unsubstituted ethylene. In embodiments, L^{6A} is independently unsubstituted methylene. In embodiments, L^{6A} is independently a bond.

In embodiments, L^{6B} is independently a bond. In embodiments, L^{6B} is independently —NHC(O)—. In embodiments, L^{6B} is independently unsubstituted arylene (e.g., C₆-C₁₂, C₆-C₁₀, or phenyl). In embodiments, L^{6B} is independently unsubstituted C₆-C₁₂ arylene. In embodiments, L^{6B} is independently unsubstituted C₆-C₁₀ arylene. In embodiments, L^{6B} is independently unsubstituted phenylene. In embodiments, L^{6B} is independently unsubstituted naphthylene. In embodiments, L^{6B} is independently unsubstituted biphenylene.

In embodiments, L^{6C} is independently a bond or unsubstituted alkylene (e.g., C₁-C₂₀, C₁-C₁₂, C₁-C₈, C₁-C₆, C₁-C₄, or C₁-C₂). In embodiments, L^{6C} is independently unsubstituted C₁-C₂₀ alkylene. In embodiments, L^{6C} is independently unsubstituted C₁-C₁₂ alkylene. In embodiments, L^{6C} is independently unsubstituted C₁-C₈ alkylene. L^{6C} is independently unsubstituted C₂-C₈ alkynylene. In embodiments, L^{6C} is independently unsubstituted C₁-C₆ alkylene. In embodiments, L^{6C} is independently unsubstituted C₁-C₄ alkylene. In embodiments, L^{6C} is independently unsubstituted ethylene. In embodiments, L^{6C} is independently unsubstituted methylene. In embodiments, L^{6C} is independently a bond or unsubstituted alkynylene (e.g., C₂-C₂₀, C₂-C₁₂, C₂-C₈, C₂-C₆, C₂-C₄, or C₂-C₂). In embodiments, L^{6C} is independently unsubstituted C₂-C₂₀ alkynylene. In embodiments, L^{6C} is independently unsubstituted C₂-C₁₂ alkynylene. In embodiments, L^{6C} is independently unsubstituted C₂-C₈ alkynylene. In embodiments, L^{6C} is independently unsubstituted C₂-C₆ alkynylene. In embodiments, L^{6C} is independently unsubstituted C₂-C₄ alkynylene. In embodiments, L^{6C} is independently unsubstituted ethynylene. In embodiments, L^{6C} is independently unsubstituted arylene (e.g., C₆-C₁₂, C₆-C₁₀, or phenyl). In embodiments, L^{6C} is independently unsubstituted C₆-C₁₂ arylene. In embodiments, L^{6C} is independently unsubstituted C₆-C₁₀ arylene. In embodiments, L^{6C} is independently unsubstituted phenylene. In embodiments, L^{6C} is independently unsubstituted naphthylene. In embodiments, L^{6C} is independently a bond.

In embodiments, L^{6D} is independently a bond or unsubstituted alkylene (e.g., C₁-C₂₀, C₁-C₁₂, C₁-C₈, C₁-C₆, C₁-C₄, or C₁-C₂). In embodiments, L^{6D} is independently unsubstituted C₁-C₂₀ alkylene. In embodiments, L^{6D} is independently unsubstituted C₁-C₁₂ alkylene. In embodiments, L^{6A} is independently unsubstituted C₁-C₈ alkylene. In embodiments, L^{6D} is independently unsubstituted C₁-C₆ alkylene. In embodiments, L^{6D} is independently unsubstituted C₁-C₄ alkylene. In embodiments, L^{6D} is independently unsubstituted ethylene. In embodiments, L^{6D} is independently unsubstituted methylene. In embodiments, L^{6D} is independently a bond.

In embodiments, L^{6E} is independently a bond. In embodiments, L^{6E} is independently —NHC(O)—.

In embodiments, L^{6A} is independently a bond or unsubstituted C₁-C₈ alkylene. In embodiments, L^{6B} is independently a bond, —NHC(O)—, or unsubstituted phenylene. In embodiments, L^{6C} is independently a bond, unsubstituted C₂-C₈ alkynylene, or unsubstituted phenylene. In embodiments, L^{6D} is independently a bond or unsubstituted C₁-C₈ alkylene. In embodiments, L^{6E} is independently a bond or —NHC(O)—.

In embodiments, L⁶ is independently a bond,

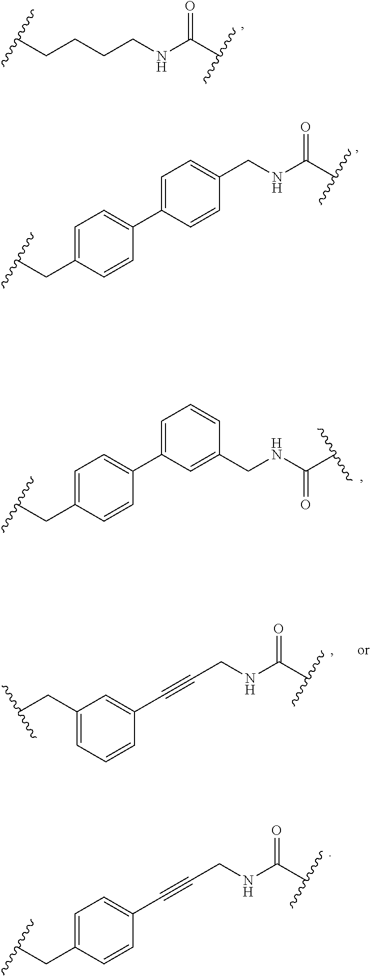 [SMILES: *CCCCNC(*)=O ; *Cc1ccc(-c2ccc(CNC(*)=O)cc2)cc1 ; *Cc1ccc(-c2cccc(CNC(*)=O)c2)cc1 ; *Cc1cccc(C#CCNC(*)=O)c1 ; *Cc1ccc(C#CCNC(*)=O)cc1]

In embodiments, L⁶ is independently a bond. In embodiments, L⁶ is independently

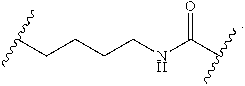 [SMILES: *CCCCNC(*)=O]

In embodiments, L⁶ is independently

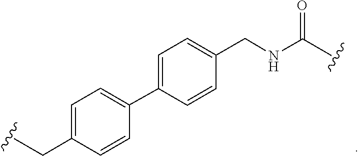

In embodiments, L⁶ is independently

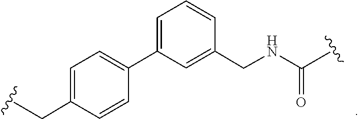

In embodiments, L⁶ is independently

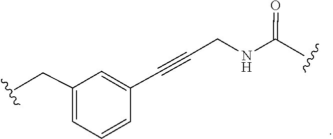

In embodiments, L⁶ is independently

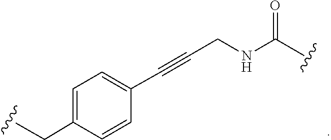

In embodiments, L⁵ is independently —NHC(O)—. In embodiments, L⁵ is independently —C(O)NH—. In embodiments, L⁵ is independently substituted or unsubstituted alkylene. In embodiments, L⁵ is independently substituted or unsubstituted heteroalkylene.

In embodiments, L⁵ is independently substituted or unsubstituted alkylene (e.g., C₁-C₂₀, C₁-C₁₂, C₁-C₈, C₁-C₆, C₁-C₄, or C₁-C₂). In embodiments, L⁵ is independently substituted alkylene (e.g., C₁-C₂₀, C₁-C₁₂, C₁-C₈, C₁-C₆, C₁-C₄, or C₁-C₂). In embodiments, L⁵ is independently unsubstituted alkylene (e.g., C₁-C₂₀, C₁-C₁₂, C₁-C₈, C₁-C₆, C₁-C₄, or C₁-C₂). In embodiments, L⁵ is independently substituted or unsubstituted C₁-C₂₀ alkylene. In embodiments, L⁵ is independently substituted C₁-C₂₀ alkylene. In embodiments, L⁵ is independently unsubstituted C₁-C₂₀ alkylene. In embodiments, L⁵ is independently substituted or unsubstituted C₁-C₁₂ alkylene. In embodiments, L⁵ is independently substituted C₁-C₁₂ alkylene. In embodiments, L⁵ is independently unsubstituted C₁-C₁₂ alkylene. In embodiments, L⁵ is independently substituted or unsubstituted C₁-C₈ alkylene. In embodiments, L⁵ is independently substituted C₁-C₈ alkylene. In embodiments, L⁵ is independently unsubstituted C₁-C₈ alkylene. In embodiments, L⁵ is independently substituted or unsubstituted C₁-C₆ alkylene. In embodiments, L⁵ is independently substituted C₁-C₆ alkylene. In embodiments, L⁵ is independently unsubstituted C₁-C₆ alkylene. In embodiments, L⁵ is independently substituted or unsubstituted C₁-C₄ alkylene. In embodiments, L⁵ is independently substituted C₁-C₄ alkylene. In embodiments, L⁵ is independently unsubstituted C₁-C₄ alkylene. In embodiments, L⁵ is independently substituted or unsubstituted ethylene. In embodiments, L⁵ is independently substituted ethylene. In embodiments, L⁵ is independently unsubstituted ethylene. In embodiments, L⁵ is independently substituted or unsubstituted methylene. In embodiments, L⁵ is independently substituted methylene. In embodiments, L⁵ is independently unsubstituted methylene.

In embodiments, L⁵ is independently substituted or unsubstituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L⁵ is independently substituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L⁵ is independently unsubstituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L⁵ is independently substituted or unsubstituted 2 to 20 membered heteroalkylene. In embodiments, L⁵ is independently substituted 2 to 20 membered heteroalkylene. In embodiments, L⁵ is independently unsubstituted 2 to 20 membered heteroalkylene. In embodiments, L⁵ is independently substituted or unsubstituted 2 to 8 membered heteroalkylene. In embodiments, L⁵ is independently substituted 2 to 8 membered heteroalkylene. In embodiments, L⁵ is independently unsubstituted 2 to 8 membered heteroalkylene. In embodiments, L⁵ is independently substituted or unsubstituted 2 to 6 membered heteroalkylene. In embodiments, L⁵ is independently substituted 2 to 6 membered heteroalkylene. In embodiments, L⁵ is independently unsubstituted 2 to 6 membered heteroalkylene. In embodiments, L⁵ is independently substituted or unsubstituted 4 to 6 membered heteroalkylene. In embodiments, L⁵ is independently substituted 4 to 6 membered heteroalkylene. In embodiments, L⁵ is independently unsubstituted 4 to 6 membered heteroalkylene. In embodiments, L⁵ is independently substituted or unsubstituted 2 to 3 membered heteroalkylene. In embodiments, L⁵ is independently substituted 2 to 3 membered heteroalkylene. In embodiments, L⁵ is independently unsubstituted 2 to 3 membered heteroalkylene. In embodiments, L⁵ is independently substituted or unsubstituted 4 to 5 membered heteroalkylene. In embodiments, L⁵ is independently substituted 4 to 5 membered heteroalkylene. In embodiments, L⁵ is independently unsubstituted 4 to 5 membered heteroalkylene.

In embodiments, L^{5A} is independently a bond or unsubstituted alkylene; L^{5B} is independently a bond, —NHC(O)—, or unsubstituted arylene; L^{5C} is independently a bond, unsubstituted alkylene, or unsubstituted arylene; L^{5D} is independently a bond or unsubstituted alkylene; and L^{5E} is independently a bond or —NHC(O)—. In embodiments, L^{5A} is independently a bond or unsubstituted alkylene. In embodiments, L^{5B} is independently a bond, —NHC(O)—, or unsubstituted arylene. In embodiments, L^{5C} is independently a bond, unsubstituted alkylene, or unsubstituted arylene. In embodiments, L^{5D} is independently a bond or unsubstituted alkylene. In embodiments, L^{5E} is independently a bond or —NHC(O)—.

In embodiments, L^{5A} is independently a bond or unsubstituted alkylene (e.g., C₁-C₂₀, C₁-C₁₂, C₁-C₈, C₁-C₆, C₁-C₄, or C₁-C₂). In embodiments, L^{5A} is independently unsubstituted C₁-C₂₀ alkylene. In embodiments, L^{5A} is independently unsubstituted C₁-C₁₂ alkylene. In embodiments, L^{5A} is independently unsubstituted C₁-C₈ alkylene. In embodiments, L^{5A} is independently unsubstituted C₁-C₆ alkylene. In embodiments, L^{5A} is independently unsubstituted C₁-C₄ alkylene. In embodiments, L^{5A} is independently unsubstituted ethylene. In embodiments, L^{5A} is independently unsubstituted methylene. In embodiments, L^{5A} is independently a bond.

In embodiments, L^{5B} is independently a bond. In embodiments, L^{5B} is independently —NHC(O)—. In embodiments, L^{5B} is independently unsubstituted arylene (e.g., C₆-C₁₂, C₆-C₁₀, or phenyl). In embodiments, L^{5B} is independently unsubstituted C₆-C₁₂ arylene. In embodiments, L^{5B} is independently unsubstituted C₆-C₁₀ arylene. In embodiments, L^{5B} is independently unsubstituted phenylene. In embodiments, L^{5B} is independently unsubstituted naphthylene.

In embodiments, L^{5C} is independently a bond or unsubstituted alkylene (e.g., C₁-C₂₀, C₁-C₁₂, C₁-C₈, C₁-C₆, C₁-C₄, or C₁-C₂). In embodiments, L^{5C} is independently unsubstituted C₁-C₂₀ alkylene. In embodiments, L^{5C} is independently unsubstituted C₁-C₁₂ alkylene. In embodiments, L^{5C} is independently unsubstituted C₁-C₈ alkylene. L^{5C} is independently unsubstituted C₂-C₈ alkynylene. In embodiments, L^{5C} is independently unsubstituted C₁-C₆ alkylene. In embodiments, L^{5C} is independently unsubstituted C₁-C₄ alkylene. In embodiments, L^{5C} is independently unsubstituted ethylene. In embodiments, L^{5C} is independently unsubstituted methylene. In embodiments, L^{5C} is independently a bond or unsubstituted alkynylene (e.g., C₂-C₂₀, C₂-C₁₂, C₂-C₈, C₂-C₆, C₂-C₄, or C₂-C₂). In embodiments, L^{5C} is independently unsubstituted C₂-C₂₀ alkynylene. In embodiments, L^{5C} is independently unsubstituted C₂-C₁₂ alkynylene. In embodiments, L^{5C} is independently unsubstituted C₂-C₈ alkynylene. In embodiments, L^{5C} is independently unsubstituted C₂-C₆ alkynylene. In embodiments, L^{5C} is independently unsubstituted C₂-C₄ alkynylene. In embodiments, L^{5C} is independently unsubstituted ethynylene. In embodiments, L^{5C} is independently unsubstituted arylene (e.g., C₆-C₁₂, C₆-C₁₀, or phenyl). In embodiments, L^{5C} is independently unsubstituted C₆-C₁₂ arylene. In embodiments, L^{5C} is independently unsubstituted C₆-C₁₀ arylene. In embodiments, L^{5C} is independently unsubstituted phenylene. In embodiments, L^{5C} is independently unsubstituted naphthylene. In embodiments, L^{5C} is independently a bond.

In embodiments, L^{5D} is independently a bond or unsubstituted alkylene (e.g., C₁-C₂₀, C₁-C₁₂, C₁-C₈, C₁-C₆, C₁-C₄, or C₁-C₂). In embodiments, L^{5D} is independently unsubstituted C₁-C₂₀ alkylene. In embodiments, L^{5D} is independently unsubstituted C₁-C₁₂ alkylene. In embodiments, L^{5A} is independently unsubstituted C₁-C₈ alkylene. In embodiments, L^{5D} is independently unsubstituted C₁-C₆ alkylene. In embodiments, L^{5D} is independently unsubstituted C₁-C₄ alkylene. In embodiments, L^{5D} is independently unsubstituted ethylene. In embodiments, L^{5D} is independently unsubstituted methylene. In embodiments, L^{5D} is independently a bond.

In embodiments, L^{5E} is independently a bond. In embodiments, L^{5E} is independently —NHC(O)—.

In embodiments, L^{5A} is independently a bond or unsubstituted C₁-C₈ alkylene. In embodiments, L^{5B} is independently a bond, —NHC(O)—, or unsubstituted phenylene. In embodiments, L^{5C} is independently a bond, unsubstituted C₂-C₈ alkynylene, or unsubstituted phenylene. In embodiments, L^{5D} is independently a bond or unsubstituted C₁-C₈ alkylene. In embodiments, L^{5E} is independently a bond or —NHC(O)—.

In embodiments, L⁵ is independently a bond,

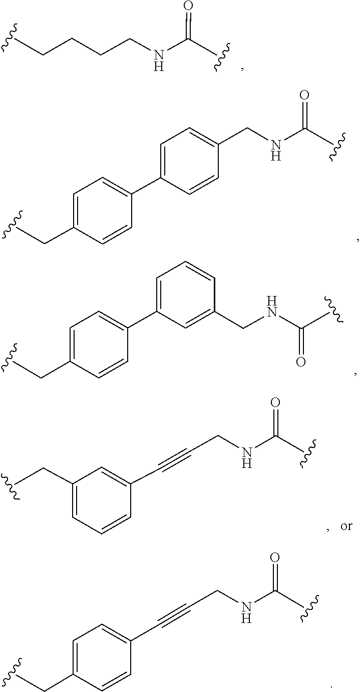

In embodiments, L⁵ is independently a bond. In embodiments, L⁵ is independently

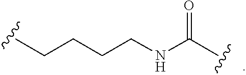

In embodiments, L⁵ is independently

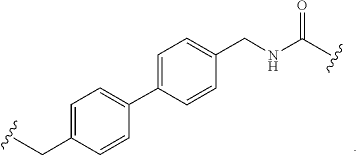

In embodiments, L⁵ is independently

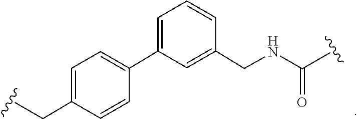

In embodiments, L⁵ is independently

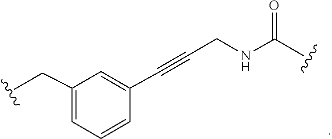

In embodiments, L⁵ is independently

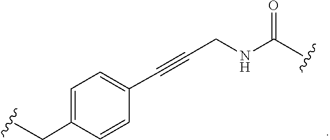

In embodiments, R¹ is unsubstituted alkyl (e.g., C₁-C₂₅, C₁-C₂₀, C₁-C₁₇, C₁-C₁₂, C₁-C₈, C₁-C₆, C₁-C₄, or C₁-C₂). In embodiments, R¹ is unsubstituted unbranched alkyl (e.g., C₁-C₂₅, C₁-C₂₀, C₁-C₁₇, C₁-C₁₂, C₁-C₈, C₁-C₆, C₁-C₄, or C₁-C₂). In embodiments, R¹ is unsubstituted unbranched saturated alkyl (e.g., C₁-C₂₅, C₁-C₂₀, C₁-C₁₇, C₁-C₁₂, C₁-C₈, C₁-C₆, C₁-C₄, or C₁-C₂). In embodiments, R¹ is unsubstituted unbranched unsaturated alkyl (e.g., C₁-C₂₅, C₁-C₂₀, C₁-C₁₇, C₁-C₁₂, C₁-C₈, C₁-C₆, C₁-C₄, or C₁-C₂).

In embodiments, R¹ is unsubstituted C₁-C₁₇ alkyl. In embodiments, R¹ is unsubstituted C₁₁-C₁₇ alkyl. In embodiments, R¹ is unsubstituted C₁₃-C₁₇ alkyl. In embodiments, R¹ is unsubstituted C₁₄-C₁₅ alkyl. In embodiments, R¹ is unsubstituted C₁₅ alkyl. In embodiments, R¹ is unsubstituted C₁₄ alkyl.

In embodiments, R¹ is unsubstituted unbranched C₁-C₁₇ alkyl. In embodiments, R¹ is unsubstituted unbranched C₁-C₁₇ alkyl. In embodiments, R¹ is unsubstituted unbranched C₁₃-C₁₇ alkyl. In embodiments, R¹ is unsubstituted unbranched C₁₄-C₁₅ alkyl. In embodiments, R¹ is unsubstituted unbranched C₁₄ alkyl. In embodiments, R¹ is unsubstituted unbranched C₁₅ alkyl.

In embodiments, R¹ is unsubstituted unbranched saturated C₁-C₁₇ alkyl. In embodiments, R¹ is unsubstituted unbranched saturated C₁-C₁₇ alkyl. In embodiments, R¹ is unsubstituted unbranched saturated C₁₃-C₁₇ alkyl. In embodiments, R¹ is unsubstituted unbranched saturated C₁₄-C₁₅ alkyl. In embodiments, R¹ is unsubstituted unbranched saturated C₁₄ alkyl. In embodiments, R¹ is unsubstituted unbranched saturated C₁₅ alkyl.

In embodiments, R¹ is unsubstituted unbranched unsaturated C₁-C₁₇ alkyl. In embodiments, R¹ is unsubstituted unbranched unsaturated C₁₁-C₁₇ alkyl. In embodiments, R¹ is unsubstituted unbranched unsaturated C₁₃-C₁₇ alkyl. In embodiments, R¹ is unsubstituted unbranched unsaturated C₁₄-C₁₅ alkyl. In embodiments, R¹ is unsubstituted unbranched unsaturated C₁₄ alkyl. In embodiments, R¹ is unsubstituted unbranched unsaturated C₁₅ alkyl.

In embodiments, R² is unsubstituted alkyl (e.g., C₁-C₂₅, C₁-C₂₀, C₁-C₁₇, C₁-C₁₂, C₁-C₈, C₁-C₆, C₁-C₄, or C₁-C₂). In embodiments, R² is unsubstituted unbranched alkyl (e.g., C₁-C₂₅, C₁-C₂₀, C₁-C₁₇, C₁-C₁₂, C₁-C₈, C₁-C₆, C₁-C₄, or C₁-C₂). In embodiments, R² is unsubstituted unbranched saturated alkyl (e.g., C₁-C₂₅, C₁-C₂₀, C₁-C₁₇, C₁-C₁₂, C₁-C₈, C₁-C₆, C₁-C₄, or C₁-C₂). In embodiments, R² is unsubstituted unbranched unsaturated alkyl (e.g., C₁-C₂₅, C₁-C₂₀, C₁-C₁₇, C₁-C₁₂, C₁-C₈, C₁-C₆, C₁-C₄, or C₁-C₂).

In embodiments, R² is unsubstituted C₁-C₁₇ alkyl. In embodiments, R² is unsubstituted C₁₁-C₁₇ alkyl. In embodiments, R² is unsubstituted C₁₃-C₁₇ alkyl. In embodiments, R² is unsubstituted C₁₄-C₁₅ alkyl. In embodiments, R² is unsubstituted C₁₄ alkyl. In embodiments, R² is unsubstituted C₁₅ alkyl.

In embodiments, R² is unsubstituted unbranched C₁-C₁₇ alkyl. In embodiments, R² is unsubstituted unbranched C₁₁-C₁₇ alkyl. In embodiments, R² is unsubstituted unbranched C₁₃-C₁₇ alkyl. In embodiments, R² is unsubstituted unbranched C₁₄-C₁₅ alkyl. In embodiments, R² is unsubstituted unbranched C₁₄ alkyl. In embodiments, R² is unsubstituted unbranched C₁₅ alkyl.

In embodiments, R² is unsubstituted unbranched saturated C₁-C₁₇ alkyl. In embodiments, R² is unsubstituted unbranched saturated C₁₁-C₁₇ alkyl. In embodiments, R² is unsubstituted unbranched saturated C₁₃-C₁₇ alkyl. In embodiments, R² is unsubstituted unbranched saturated C₁₄-C₁₅ alkyl. In embodiments, R² is unsubstituted unbranched saturated C₁₄ alkyl. In embodiments, R² is unsubstituted unbranched saturated C₁₅ alkyl.

In embodiments, R² is unsubstituted unbranched unsaturated C₁-C₁₇ alkyl. In embodiments, R² is unsubstituted unbranched unsaturated C₁-C₁₇ alkyl. In embodiments, R² is unsubstituted unbranched unsaturated C₁₃-C₁₇ alkyl. In embodiments, R² is unsubstituted unbranched unsaturated C₁₄-C₁₅ alkyl. In embodiments, R² is unsubstituted unbranched unsaturated C₁₄ alkyl. In embodiments, R² is unsubstituted unbranched unsaturated C₁₅ alkyl.

In embodiments, at least one of R¹ and R² is unsubstituted C₁-C₁₉ alkyl. In embodiments, at least one of R¹ and R² is unsubstituted C₉-C₁₉ alkyl. In embodiments, at least one of R¹ and R² is unsubstituted C₁₁-C₁₉ alkyl. In embodiments, at least one of R¹ and R² is unsubstituted C₁₃-C₁₉ alkyl.

In embodiments, R¹ is unsubstituted C₁-C₁₉ alkyl. In embodiments, R¹ is unsubstituted C₉-C₁₉ alkyl. In embodiments, R¹ is unsubstituted C₁₁-C₁₉ alkyl. In embodiments, R¹ is unsubstituted C₁₃-C₁₉ alkyl. In embodiments, R¹ is unsubstituted unbranched C₁-C₁₉ alkyl. In embodiments, R¹ is unsubstituted unbranched C₉-C₁₉ alkyl. In embodiments, R¹ is unsubstituted unbranched C₁₁-C₁₉ alkyl. In embodiments, R¹ is unsubstituted unbranched C₁₃-C₁₉ alkyl. In embodiments, R¹ is unsubstituted unbranched saturated C₁-C₁₉ alkyl. In embodiments, R¹ is unsubstituted unbranched saturated C₉-C₁₉ alkyl. In embodiments, R¹ is unsubstituted unbranched saturated C₁₁-C₁₉ alkyl. In embodiments, R¹ is unsubstituted unbranched saturated C₁₃-C₁₉ alkyl. In embodiments, R¹ is unsubstituted unbranched unsaturated C₁-C₁₉ alkyl. In embodiments, R¹ is unsubstituted unbranched unsaturated C₉-C₁₉ alkyl. In embodiments, R¹ is unsubstituted unbranched unsaturated C₁₁-C₁₉ alkyl. In embodiments, R¹ is unsubstituted unbranched unsaturated C₁₃-C₁₉ alkyl.

In embodiments, R² is unsubstituted C₁-C₁₉ alkyl. In embodiments, R² is unsubstituted C₉-C₁₉ alkyl. In embodiments, R² is unsubstituted C₁₁-C₁₉ alkyl. In embodiments, R² is unsubstituted C₁₃-C₁₉ alkyl. In embodiments, R² is unsubstituted unbranched C₁-C₁₉ alkyl. In embodiments, R² is unsubstituted unbranched C₉-C₁₉ alkyl. In embodiments, R² is unsubstituted unbranched C₁₁-C₁₉ alkyl. In embodiments, R² is unsubstituted unbranched C₁₃-C₁₉ alkyl. In embodiments, R² is unsubstituted unbranched saturated C₁-C₁₉ alkyl. In embodiments, R² is unsubstituted unbranched saturated C₉-C₁₉ alkyl. In embodiments, R² is unsubstituted unbranched saturated C₁₁-C₁₉ alkyl. In embodiments, R² is unsubstituted unbranched saturated C₁₃-C₁₉ alkyl. In embodiments, R² is unsubstituted unbranched unsaturated C₁-C₁₉ alkyl. In embodiments, R² is unsubstituted unbranched unsaturated C₉-C₁₉ alkyl. In embodiments, R² is unsubstituted unbranched unsaturated C₁₁-C₁₉ alkyl. In embodiments, R² is unsubstituted unbranched unsaturated C₁₃-C₁₉ alkyl.

L^{1A} is independently a bond, —N(R²⁰)—, —O—, —S—, —C(O)—, —N(R²⁰)C(O)—, —C(O)N(R²¹)—, —N(R²⁰)C(O)N(R²¹)—, —C(O)O—, —OC(O)—, —N(R²⁰)C(O)O—, —OC(O)N(R²¹)—, —OPO₂—O—, —O—P(O)(S)—O—, —O—P(O)(R²²)—O—, —O—P(S)(R²²)—O—, —O—P(O)(NR²⁰R²¹)—N—, —O—P(S)(NR²⁰R²¹)—N—, —O—P(O)(NR²⁰R²¹)—O—, —O—P(S)(NR²⁰R²¹)—O—, —P(O)(NR²⁰R²¹)—N—, —P(S)(NR²⁰R²¹)—N—, —P(O)(NR²⁰R²¹)—O—, —P(S)(NR²⁰R²¹)—O—, —S—S—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L^{1A} is independently a bond, —N(R²⁰)—, —O—, —S—, —C(O)—, —N(R²⁰)C(O)—, —C(O)N(R²¹)—, —N(R²⁰)C(O)N(R²¹)—, —C(O)O—, —OC(O)—, —N(R²⁰)C(O)O—, —OC(O)N(R²¹)—, —OPO₂—O—, —O—P(O)(S)—O—, —O—P(O)(R²²)—O—, —O—P(S)(R²²)—O—, —O—P(O)(NR²⁰R²¹)—N—, —O—P(S)(NR²⁰R²¹)—N—, —O—P(O)(NR²⁰R²¹)—O—, —O—P(S)(NR²⁰R²¹)—O—, —P(O)(NR²⁰R²¹)—N—, —P(S)(NR²⁰R²¹)—N—, —P(O)(NR²⁰R²¹)—O—, —P(S)(NR²⁰R²¹)—O—, —S—S—, substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L^{1A} is independently a bond, —N(R²⁰)—, —O—, —S—, —C(O)—, —N(R²⁰)C(O)—, —C(O)N(R²¹)—, —N(R²⁰)C(O)N(R²¹)—, —C(O)O—, —OC(O)—, —N(R²⁰)C(O)O—, —OC(O)N(R²¹)—, —OPO₂—O—, —O—P(O)(S)—O—, —O—P(O)(R²²)—O—, —O—P(S)(R²²)—O—, —O—P(O)(NR²⁰R²¹)—N—, —O—P(S)(NR²⁰R²¹)—N—, —O—P(O)(NR²⁰R²¹)—O—, —O—P(S)(NR²⁰R²¹)—O—, —P(O)(NR²⁰R²¹)—N—, —P(S)(NR²⁰R²¹)—N—, —P(O)(NR²⁰R²¹)—O—, —P(S)(NR²⁰R²¹)—O—, —S—S—, unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L^{1A} is substituted, L^{1A} is substituted with a substituent group. In embodiments, when L^{1A} is substituted, L^{1A} is substituted with a size-limited substituent group. In embodiments, when L^{1A} is substituted, L^{1A} is substituted with a lower substituent group.

L^{1B} is independently a bond, —N(R²⁰)—, —O—, —S—, —C(O)—, —N(R²⁰)C(O)—, —C(O)N(R²¹)—, —N(R²⁰)C(O)N(R²¹)—, —C(O)O—, —OC(O)—, —N(R²⁰)C(O)O—, —OC(O)N(R²¹)—, —OPO₂—O—, —O—P(O)(S)—O—, —O—P(O)(R²²)—O—, —O—P(S)(R²²)—O—, —O—P(O)(NR²⁰R²¹)—N—, —O—P(S)(NR²⁰R²¹)—N—, —O—P(O)(NR²⁰R²¹)—O—, —O—P(S)(NR²⁰R²¹)—O—, —P(O)(NR²⁰R²¹)—N—, —P(S)(NR²⁰R²¹)—N—, —P(O)(NR²⁰R²¹)—O—, —P(S)(NR²⁰R²¹)—O—, —S—S—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L^{1B} is independently a bond, —N(R²⁰)—, —O—, —S—, —C(O)—, —N(R²⁰)C(O)—, —C(O)N(R²¹)—, —N(R²⁰)C(O)N(R²¹)—, —C(O)O—, —OC(O)—, —N(R²⁰)C(O)O—, —OC(O)N(R²¹)—, —OPO₂—O—, —O—P(O)(S)—O—, —O—P(O)(R²²)—O—, —O—P(S)(R²²)—O—, —O—P(O)(NR²⁰R²¹)—N—, —O—P(S)(NR²⁰R²¹)—N—, —O—P(O)(NR²⁰R²¹)—O—, —O—P(S)(NR²⁰R²¹)—O—, —P(O)(NR²⁰R²¹)—N—, —P(S)(NR²⁰R²¹)—N—, —P(O)(NR²⁰R²¹)—O—, —P(S)(NR²⁰R²¹)—O—, —S—S—, substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L^{1B} is independently a bond, —N(R²⁰)—, —O—, —S—, —C(O)—, —N(R²⁰)C(O)—, —C(O)N(R²¹)—, —N(R²⁰)C(O)N(R²¹)—, —C(O)O—, —OC(O)—, —N(R²⁰)C(O)O—, —OC(O)N(R²¹)—, —OPO₂—O—, —O—P(O)(S)—O—, —O—P(O)(R²²)—O—, —O—P(S)(R²²)—O—, —O—P(O)(NR²⁰R²¹)—N—, —O—P(S)(NR²⁰R²¹)—N—, —O—P(O)(NR²⁰R²¹)—O—, —O—P(S)(NR²⁰R²¹)—O—, —P(O)(NR²⁰R²¹)—N—, —P(S)(NR²⁰R²¹)—N—, —P(O)(NR²⁰R²¹)—O—, —P(S)(NR²⁰R²¹)—O—, —S—S—, unsubstituted alkylene (e.g., C₁-C₂₀, C₁-C₁₂, C₁-C₈, C₁-C₆, C₁-C₄, or C₁-C₂), unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L^{1B} is substituted, L^{1B} is substituted with a substituent group. In embodiments, when L^{1B} is substituted, L^{1B} is substituted with a size-limited substituent group. In embodiments, when L^{1B} is substituted, L^{1B} is substituted with a lower substituent group.

L^{1C} is independently a bond, —N(R²⁰)—, —O—, —S—, —C(O)—, —N(R²⁰)C(O)—, —C(O)N(R²¹)—, —N(R²⁰)C(O)N(R²¹)—, —C(O)O—, —OC(O)—, —N(R²⁰)C(O)O—, —OC(O)N(R²¹)—, —OPO₂—O—, —O—P(O)(S)—O—, —O—P(O)(R²²)—O—, —O—P(S)(R²²)—O—, —O—P(O)(NR²⁰R²¹)—N—, —O—P(S)(NR²⁰R²¹)—N—, —O—P(O)(NR²⁰R²¹)—O—, —O—P(S)(NR²⁰R²¹)—O—, —P(O)(NR²⁰R²¹)—N—, —P(S)(NR²⁰R²¹)—N—, —P(O)(NR²⁰R²¹)—O—, —P(S)(NR²⁰R²¹)—O—, —S—S—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L^{1C} is independently a bond, —N(R²⁰)—, —O—, —S—, —C(O)—, —N(R²⁰)C(O)—, —C(O)N(R²¹)—, —N(R²⁰)C(O)N(R²¹)—, —C(O)O—, —OC(O)—, —N(R²⁰)C(O)O—, —OC(O)N(R²¹)—, —OPO₂—O—, —O—P(O)(S)—O—, —O—P(O)(R²²)—O—, —O—P(S)(R²²)—O—, —O—P(O)(NR²⁰R²¹)—N—, —O—P(S)(NR²⁰R²¹)—N—, —O—P(O)(NR²⁰R²¹)—O—, —O—P(S)(NR²⁰R²¹)—O—, —P(O)(NR²⁰R²¹)—N—, —P(S)(NR²⁰R²¹)—N—, —P(O)(NR²⁰R²¹)—O—, —P(S)(NR²⁰R²¹)—O—, —S—S—, substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L^{1C} is independently a bond, —N(R²⁰)—, —O—, —S—, —C(O)—, —N(R²⁰)C(O)—, —C(O)N(R²¹)—, —N(R²⁰)C(O)N(R²¹)—, —C(O)O—, —OC(O)—, —N(R²⁰)C(O)O—, —OC(O)N(R²¹)—, —OPO₂—O—, —O—P(O)(S)—O—, —O—P(O)(R²²)—O—, —O—P(S)(R²²)—O—, —O—P(O)(NR²⁰R²¹)—N—, —O—P(S)(NR²⁰R²¹)—N—, —O—P(O)(NR²⁰R²¹)—O—, —O—P(S)(NR²⁰R²¹)—O—, —P(O)(NR²⁰R²¹)—N—, —P(S)(NR²⁰R²¹)—N—, —P(O)(NR²⁰R²¹)—O—, —P(S)(NR²⁰R²¹)—O—, —S—S—, unsubstituted alkylene (e.g., C₁-C₂₀, C₁-C₁₂, C₁-C₈, C₁-C₆, C₁-C₄, or C₁-C₂), unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L^{1C} is substituted, L^{1C} is substituted with a substituent group. In embodiments, when L^{1C} is substituted, L^{1C} is substituted with a size-limited substituent group. In embodiments, when L^{1C} is substituted, L^{1C} is substituted with a lower substituent group.

R^{1C} is independently substituted (e.g., substituted with a substituent group, a size-limited substituent group, or lower substituent group) or unsubstituted alkyl (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkyl (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkyl (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkyl (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted aryl (e.g., C₆-C₁₂, C₆-C₁₀, or phenyl), or substituted (e.g., substituted with a substituent group, a size-limited substituent group, or lower substituent group) or unsubstituted heteroaryl (e.g., 5 to 12 membered, 5 to 10 membered, 5 to 9 membered, or 5 to 6 membered). In embodiments, R^{1C} is independently substituted (e.g., substituted with a substituent group, a size-limited substituent group, or lower substituent group) alkyl (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkyl (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkyl (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkyl (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) aryl (e.g., C₆-C₁₂, C₆-C₁₀, or phenyl), or substituted (e.g., substituted with a substituent group, a size-limited substituent group, or lower substituent group) heteroaryl (e.g., 5 to 12 membered, 5 to 10 membered, 5 to 9 membered, or 5 to 6 membered). In embodiments, R^{1C} is independently unsubstituted alkyl (e.g., C₁-C₂₀, C₁-C₁₂, C₁-C₈, C₁-C₆, C₁-C₄, or C₁-C₂), unsubstituted heteroalkyl (e.g., 2 to 20 membered, 2 to 12 membered, 2 to 8 membered, 2 to 6 membered, 4 to 6 membered, 2 to 3 membered, or 4 to 5 membered), unsubstituted cycloalkyl (e.g., C₃-C₁₀, C₃-C₈, C₃-C₆, C₄-C₆, or C₅-C₆), unsubstituted heterocycloalkyl (e.g., 3 to 10 membered, 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). In embodiments, when R^{1C} is substituted, R^{1C} is substituted with a substituent group. In embodiments, when R^{1C} is substituted, R^{1C} is substituted with a size-limited substituent group. In embodiments, when R^{1C} is substituted, R^{1C} is substituted with a lower substituent group. In embodiments, R^{1C} is substituted with oxo (═O).

L^{1D} is independently a bond, —N(R²⁰)—, —O—, —S—, —C(O)—, —N(R²⁰)C(O)—, —C(O)N(R²¹)—, —N(R²⁰)C(O)N(R²¹)—, —C(O)O—, —OC(O)—, —N(R²⁰)C(O)O—, —OC(O)N(R²¹)—, —OPO₂—O—, —O—P(O)(S)—O—, —O—P(O)(R²²)—O—, —O—P(S)(R²²)—O—, —O—P(O)(NR²⁰R²¹)—N—, —O—P(S)(NR²⁰R²¹)—N—, —O—P(O)(NR²⁰R²¹)—O—, —O—P(S)(NR²⁰R²¹)—O—, —P(O)(NR²⁰R²¹)—N—, —P(S)(NR²⁰R²¹)—N—, —P(O)(NR²⁰R²¹)—O—, —P(S)(NR²⁰R²¹)—O—, —S—S—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L^{1D} is independently a bond, —N(R²⁰)—, —O—, —S—, —C(O)—, —N(R²⁰)C(O)—, —C(O)N(R²¹)—, —N(R²⁰)C(O)N(R²¹)—, —C(O)O—, —OC(O)—, —N(R²⁰)C(O)O—, —OC(O)N(R²¹)—, —OPO₂—O—, —O—P(O)(S)—O—, —O—P(O)(R²²)—O—, —O—P(S)(R²²)—O—, —O—P(O)(NR²⁰R²¹)—N—, —O—P(S)(NR²⁰R²¹)—N—, —O—P(O)(NR²⁰R²¹)—O—, —O—P(S)(NR²⁰R²¹)—O—, —P(O)(NR²⁰R²¹)—N—, —P(S)(NR²⁰R²¹)—N—, —P(O)(NR²⁰R²¹)—O—, —P(S)(NR²⁰R²¹)—O—, —S—S—, substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L^{1D} is independently a bond, —N(R²⁰)—, —O—, —S—, —C(O)—, —N(R²⁰)C(O)—, —C(O)N(R²¹)—, —N(R²⁰)C(O)N(R²¹)—, —C(O)O—, —OC(O)—, —N(R²⁰)C(O)O—, —OC(O)N(R²¹)—, —OPO₂—O—, —O—P(O)(S)—O—, —O—P(O)(R²²)—O—, —O—P(S)(R²²)—O—, —O—P(O)(NR²⁰R²¹)—N—, —O—P(S)(NR²⁰R²¹)—N—, —O—P(O)(NR²⁰R²¹)—O—, —O—P(S)(NR²⁰R²¹)—O—, —P(O)(NR²⁰R²¹)—N—, —P(S)(NR²⁰R²¹)—N—, —P(O)(NR²⁰R²¹)—O—, —P(S)(NR²⁰R²¹)—O—, —S—S—, unsubstituted alkylene (e.g., C₁-C₂₀, C₁-C₁₂, C₁-C₈, C₁-C₆, C₁-C₄, or C₁-C₂), unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L^{1D} is substituted, L^{1D} is substituted with a substituent group. In embodiments, when L^{1D} is substituted, L^{1D} is substituted with a size-limited substituent group. In embodiments, when L^{1D} is substituted, L^{1D} is substituted with a lower substituent group.

R^{1D} is independently substituted (e.g., substituted with a substituent group, a size-limited substituent group, or lower substituent group) or unsubstituted alkyl (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkyl (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkyl (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkyl (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted aryl (e.g., C₆-C₁₂, C₆-C₁₀, or phenyl), or substituted (e.g., substituted with a substituent group, a size-limited substituent group, or lower substituent group) or unsubstituted heteroaryl (e.g., 5 to 12 membered, 5 to 10 membered, 5 to 9 membered, or 5 to 6 membered). In embodiments, R^{1D} is independently substituted (e.g., substituted with a substituent group, a size-limited substituent group, or lower substituent group) alkyl (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkyl (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkyl (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkyl (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) aryl (e.g., C₆-C₁₂, C₆-C₁₀, or phenyl), or substituted (e.g., substituted with a substituent group, a size-limited substituent group, or lower substituent group) heteroaryl (e.g., 5 to 12 membered, 5 to 10 membered, 5 to 9 membered, or 5 to 6 membered). In embodiments, R^{1D} is independently unsubstituted alkyl (e.g., C₁-C₂₀, C₁-C₁₂, C₁-C₈, C₁-C₆, C₁-C₄, or C₁-C₂), unsubstituted heteroalkyl (e.g., 2 to 20 membered, 2 to 12 membered, 2 to 8 membered, 2 to 6 membered, 4 to 6 membered, 2 to 3 membered, or 4 to 5 membered), unsubstituted cycloalkyl (e.g., C₃-C₁₀, C₃-C₈, C₃-C₆, C₄-C₆, or C₅-C₆), unsubstituted heterocycloalkyl (e.g., 3 to 10 membered, 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). In embodiments, when R^{1D} is substituted, R^{1D} is substituted with a substituent group. In embodiments, when R^{1D} is substituted, R^{1D} is substituted with a size-limited substituent group. In embodiments, when R^{1D} is substituted, R^{1D} is substituted with a lower substituent group.

L^{1E} is independently a bond, —N(R²⁰)—, —O—, —S—, —C(O)—, —N(R²⁰)C(O)—, —C(O)N(R²¹)—, —N(R²⁰)C(O)N(R²¹)—, —C(O)O—, —OC(O)—, —N(R²⁰)C(O)O—, —OC(O)N(R²¹)—, —OPO₂—O—, —O—P(O)(S)—O—, —O—P(O)(R²²)—O—, —O—P(S)(R²²)—O—, —O—P(O)(NR²⁰R²¹)—N—, —O—P(S)(NR²⁰R²¹)—N—, —O—P(O)(NR²⁰R²¹)—O—, —O—P(S)(NR²⁰R²¹)—O—, —P(O)(NR²⁰R²¹)—N—, —P(S)(NR²⁰R²¹)—N—, —P(O)(NR²⁰R²¹)—O—, —P(S)(NR²⁰R²¹)—O—, —S—S—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L^{1E} is independently a bond, —N(R²⁰)—, —O—, —S—, —C(O)—, —N(R²⁰)C(O)—, —C(O)N(R²¹)—, —N(R²⁰)C(O)N(R²¹)—, —C(O)O—, —OC(O)—, —N(R²⁰)C(O)O—, —OC(O)N(R²¹)—, —OPO₂—O—, —O—P(O)(S)—O—, —O—P(O)(R²²)—O—, —O—P(S)(R²²)—O—, —O—P(O)(NR²⁰R²¹)—N—, —O—P(S)(NR²⁰R²¹)—N—, —O—P(O)(NR²⁰R²¹)—O—, —O—P(S)(NR²⁰R²¹)—O—, —P(O)(NR²⁰R²¹)—N—, —P(S)(NR²⁰R²¹)—N—, —P(O)(NR²⁰R²¹)—O—, —P(S)(NR²⁰R²¹)—O—, —S—S—, substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L^{1E} is independently a bond, —N(R²⁰)—, —O—, —S—, —C(O)—, —N(R²⁰)C(O)—, —C(O)N(R²¹)—, —N(R²⁰)C(O)N(R²¹)—, —C(O)O—, —OC(O)—, —N(R²⁰)C(O)O—, —OC(O)N(R²¹)—, —OPO₂—O—, —O—P(O)(S)—O—, —O—P(O)(R²²)—O—, —O—P(S)(R²²)—O—, —O—P(O)(NR²⁰R²¹)—N—, —O—P(S)(NR²⁰R²¹)—N—, —O—P(O)(NR²⁰R²¹)—O—, —O—P(S)(NR²⁰R²¹)—O—, —P(O)(NR²⁰R²¹)—N—, —P(S)(NR²⁰R²¹)—N—, —P(O)(NR²⁰R²¹)—O—, —P(S)(NR²⁰R²¹)—O—, —S—S—, unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L^{1E} is substituted, L^{1E} is substituted with a substituent group. In embodiments, when L^{1E} is substituted, L^{1E} is substituted with a size-limited substituent group. In embodiments, when L^{1E} is substituted, L^{1E} is substituted with a lower substituent group.

R^{1E} is independently substituted (e.g., substituted with a substituent group, a size-limited substituent group, or lower substituent group) or unsubstituted alkyl (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkyl (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkyl (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkyl (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted aryl (e.g., C₆-C₁₂, C₆-C₁₀, or phenyl), or substituted (e.g., substituted with a substituent group, a size-limited substituent group, or lower substituent group) or unsubstituted heteroaryl (e.g., 5 to 12 membered, 5 to 10 membered, 5 to 9 membered, or 5 to 6 membered). In embodiments, R^{1E} is independently substituted (e.g., substituted with a substituent group, a size-limited substituent group, or lower substituent group) alkyl (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkyl (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkyl (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkyl (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) aryl (e.g., C₆-C₁₂, C₆-C₁₀, or phenyl), or substituted (e.g., substituted with a substituent group, a size-limited substituent group, or lower substituent group) heteroaryl (e.g., 5 to 12 membered, 5 to 10 membered, 5 to 9 membered, or 5 to 6 membered). In embodiments, R^{1E} is independently unsubstituted alkyl (e.g., C₁-C₂₀, C₁-C₁₂, C₁-C₈, C₁-C₆, C₁-C₄, or C₁-C₂), unsubstituted heteroalkyl (e.g., 2 to 20 membered, 2 to 12 membered, 2 to 8 membered, 2 to 6 membered, 4 to 6 membered, 2 to 3 membered, or 4 to 5 membered), unsubstituted cycloalkyl (e.g., C₃-C₁₀, C₃-C₈, C₃-C₆, C₄-C₆, or C₅-C₆), unsubstituted heterocycloalkyl (e.g., 3 to 10 membered, 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). In embodiments, when R^{1E} is substituted, R^{1E} is substituted with a substituent group. In embodiments, when R^{1E} is substituted, R^{1E} is substituted with a size-limited substituent group. In embodiments, when R^{1E} is substituted, R^{1E} is substituted with a lower substituent group.

R²⁰ is independently hydrogen or unsubstituted alkyl (e.g., C₁-C₂₀, C₁-C₁₂, C₁-C₈, C₁-C₆, C₁-C₄, or C₁-C₂). In embodiments, R²⁰ is independently hydrogen. In embodiments, R²⁰ is independently unsubstituted C₁-C₂₀ alkyl. In embodiments, R²⁰ is independently hydrogen or unsubstituted C₁-C₁₂ alkyl. In embodiments, R²⁰ is independently hydrogen or unsubstituted C₁-C₁₀ alkyl. In embodiments, R²⁰ is independently hydrogen or unsubstituted C₁-C₈ alkyl. In embodiments, R²⁰ is independently hydrogen or unsubstituted C₁-C₆ alkyl. In embodiments, R²⁰ is independently hydrogen or unsubstituted C₁-C₄ alkyl. In embodiments, R²⁰ is independently hydrogen or unsubstituted C₁-C₂ alkyl.

R²¹ is independently hydrogen or unsubstituted alkyl (e.g., C₁-C₂₀, C₁-C₁₂, C₁-C₈, C₁-C₆, C₁-C₄, or C₁-C₂). In embodiments, R²¹ is independently hydrogen. In embodiments, R²¹ is independently unsubstituted C₁-C₂₀ alkyl. In embodiments, R²¹ is independently hydrogen or unsubstituted C₁-C₁₂ alkyl. In embodiments, R²¹ is independently hydrogen or unsubstituted C₁-C₁₀ alkyl. In embodiments, R²¹ is independently hydrogen or unsubstituted C₁-C₈ alkyl. In embodiments, R²¹ is independently hydrogen or unsubstituted C₁-C₆ alkyl. In embodiments, R²¹ is independently hydrogen or unsubstituted C₁-C₄ alkyl. In embodiments, R²¹ is independently hydrogen or unsubstituted C₁-C₂ alkyl.

R²² is independently hydrogen or unsubstituted alkyl (e.g., C₁-C₂₀, C₁-C₁₂, C₁-C₈, C₁-C₆, C₁-C₄, or C₁-C₂). In embodiments, R²² is independently hydrogen. In embodiments, R²² is independently unsubstituted C₁-C₂₀ alkyl. In embodiments, R²² is independently hydrogen or unsubstituted C₁-C₁₂ alkyl. In embodiments, R²² is independently hydrogen or unsubstituted C₁-C₁₀ alkyl. In embodiments, R²² is independently hydrogen or unsubstituted C₁-C₈ alkyl. In embodiments, R²² is independently hydrogen or unsubstituted C₁-C₆ alkyl. In embodiments, R²² is independently hydrogen or unsubstituted C₁-C₄ alkyl. In embodiments, R²² is independently hydrogen or unsubstituted C₁-C₂ alkyl.

L² is independently an unsubstituted alkylene (e.g., C₁-C₃₀, C₅-C₂₅, C₁₀-C₂₅, C₁₀-C₂₄, C₁-C₁₀, C₁-C₈, C₁-C₆, C₁-C₄, or C₁-C₂). In embodiments, L² is independently an unsubstituted C₁-C₃₀ alkylene. In embodiments, L² is independently an unsubstituted C₅-C₂₅ alkylene. In embodiments, L² is independently an unsubstituted C₁₀-C₂₅ alkylene. In embodiments, L² is independently an unsubstituted C₁₀-C₂₄ alkylene. In embodiments, L² is independently an unsubstituted C₁-C₁₀ alkylene. In embodiments, L² is independently an unsubstituted C₁-C₈ alkylene. In embodiments, L² is independently an unsubstituted C₁-C₆ alkylene. In embodiments, L² is independently an unsubstituted C₁-C₄ alkylene. In embodiments, L² is independently an unsubstituted C₁-C₃ alkylene. In embodiments, L² is independently an unsubstituted C₁-C₂ alkylene.

In embodiments, L² is independently an unsubstituted unbranched C₁-C₃₀ alkylene. In embodiments, L² is independently an unsubstituted unbranched C₅-C₂₅ alkylene. In embodiments, L² is independently an unsubstituted unbranched C₁₀-C₂₅ alkylene. In embodiments, L² is independently an unsubstituted unbranched C₁₀-C₂₄ alkylene. In embodiments, L² is independently an unsubstituted unbranched C₁-C₁₀ alkylene. In embodiments, L² is independently an unsubstituted unbranched C₁-C₈ alkylene. In embodiments, L² is independently an unsubstituted unbranched C₁-C₆ alkylene. In embodiments, L² is independently an unsubstituted unbranched C₁-C₄ alkylene. In embodiments, L² is independently an unsubstituted unbranched C₁-C₃ alkylene. In embodiments, L² is independently an unsubstituted unbranched C₁-C₂ alkylene.

In embodiments, L² is independently an unsubstituted unbranched saturated C₁-C₃₀ alkylene. In embodiments, L² is independently an unsubstituted unbranched saturated C₅-C₂₅ alkylene. In embodiments, L² is independently an unsubstituted unbranched saturated C₁₀-C₂₅ alkylene. In embodiments, L² is independently an unsubstituted unbranched saturated C₁₀-C₂₄ alkylene. In embodiments, L² is independently an unsubstituted unbranched saturated C₁-C₁₀ alkylene. In embodiments, L² is independently an unsubstituted unbranched saturated C₁-C₈ alkylene. In embodiments, L² is independently an unsubstituted unbranched saturated C₁-C₆ alkylene. In embodiments, L² is independently an unsubstituted unbranched saturated C₁-C₄ alkylene. In embodiments, L² is independently an unsubstituted unbranched saturated C₁-C₃ alkylene. In embodiments, L² is independently an unsubstituted unbranched saturated C₁-C₂ alkylene.

In embodiments, L² is independently an unsubstituted unbranched unsaturated C₁-C₃₀ alkylene. In embodiments, L² is independently an unsubstituted unbranched unsaturated C₅-C₂₅ alkylene. In embodiments, L² is independently an unsubstituted unbranched unsaturated C₁₀-C₂₅ alkylene. In embodiments, L² is independently an unsubstituted unbranched unsaturated C₁₀-C₂₄ alkylene. In embodiments, L² is independently an unsubstituted unbranched unsaturated C₁-C₁₀ alkylene. In embodiments, L² is independently an unsubstituted unbranched unsaturated C₁-C₈ alkylene. In embodiments, L² is independently an unsubstituted unbranched unsaturated C₁-C₆ alkylene. In embodiments, L² is independently an unsubstituted unbranched unsaturated C₁-C₄ alkylene. In embodiments, L² is independently an unsubstituted unbranched unsaturated C₁-C₃ alkylene. In embodiments, L² is independently an unsubstituted unbranched unsaturated C₁-C₂ alkylene.

L^{2A} is independently a bond or an unsubstituted alkylene (e.g., C₁-C₃₀, C₅-C₂₅, C₁₀-C₂₅, C₁₀-C₂₄, C₁-C₁₀, C₁-C₈, C₁-C₆, C₁-C₄, or C₁-C₂). In embodiments, L^{2A} is independently an unsubstituted C₁-C₃₀ alkylene. In embodiments, L^{2A} is independently an unsubstituted C₅-C₂₅ alkylene. In embodiments, L^{2A} is independently an unsubstituted C₁₀-C₂₅ alkylene. In embodiments, L^{2A} is independently an unsubstituted C₁₀-C₂₄ alkylene. In embodiments, L^{2A} is independently an unsubstituted C₁-C₁₀ alkylene. In embodiments, L^{2A} is independently an unsubstituted C₁-C₈ alkylene. In embodiments, L^{2A} is independently an unsubstituted C₁-C₆ alkylene. In embodiments, L^{2A} is independently an unsubstituted C₁-C₄ alkylene. In embodiments, L^{2A} is independently an unsubstituted C₁-C₃ alkylene. In embodiments, L^{2A} is independently an unsubstituted C₁-C₂ alkylene.

In embodiments, L^{2A} is independently an unsubstituted unbranched C₁-C₃₀ alkylene. In embodiments, L^{2A} is independently an unsubstituted unbranched C₅-C₂₅ alkylene. In embodiments, L^{2A} is independently an unsubstituted unbranched C₁₀-C₂₅ alkylene. In embodiments, L^{2A} is independently an unsubstituted unbranched C₁₀-C₂₄ alkylene. In embodiments, L^{2A} is independently an unsubstituted unbranched C₁-C₁₀ alkylene. In embodiments, L^{2A} is independently an unsubstituted unbranched C₁-C₈ alkylene. In embodiments, L^{2A} is independently an unsubstituted unbranched C₁-C₆ alkylene. In embodiments, L^{2A} is independently an unsubstituted unbranched C₁-C₄ alkylene. In embodiments, L^{2A} is independently an unsubstituted unbranched C₁-C₃ alkylene. In embodiments, L^{2A} is independently an unsubstituted unbranched C₁-C₂ alkylene.

In embodiments, L^{2A} is independently an unsubstituted unbranched saturated C₁-C₃₀ alkylene. In embodiments, L^{2A} is independently an unsubstituted unbranched saturated C₅-C₂₅ alkylene. In embodiments, L^{2A} is independently an unsubstituted unbranched saturated C₁₀-C₂₅ alkylene. In embodiments, L^{2A} is independently an unsubstituted unbranched saturated C₁₀-C₂₄ alkylene. In embodiments, L^{2A} is independently an unsubstituted unbranched saturated C₁-C₁₀ alkylene. In embodiments, L^{2A} is independently an unsubstituted unbranched saturated C₁-C₈ alkylene. In embodiments, L^{2A} is independently an unsubstituted unbranched saturated C₁-C₆ alkylene. In embodiments, L^{2A} is independently an unsubstituted unbranched saturated C₁-C₄ alkylene. In embodiments, L^{2A} is independently an unsubstituted unbranched saturated C₁-C₃ alkylene. In embodiments, L^{2A} is independently an unsubstituted unbranched saturated C₁-C₂ alkylene.

In embodiments, L^{2A} is independently an unsubstituted unbranched unsaturated C₁-C₃₀ alkylene. In embodiments, L^{2A} is independently an unsubstituted unbranched unsaturated C₅-C₂₅ alkylene. In embodiments, L^{2A} is independently an unsubstituted unbranched unsaturated C₁₀-C₂₅ alkylene. In embodiments, L^{2A} is independently an unsubstituted unbranched unsaturated C₁₀-C₂₄ alkylene. In embodiments, L^{2A} is independently an unsubstituted unbranched unsaturated C₁-C₁₀ alkylene. In embodiments, L^{2A} is independently an unsubstituted unbranched unsaturated C₁-C₈ alkylene. In embodiments, L^{2A} is independently an unsubstituted unbranched unsaturated C₁-C₆ alkylene. In embodiments, L^{2A} is independently an unsubstituted unbranched unsaturated C₁-C₄ alkylene. In embodiments, L^{2A} is independently an unsubstituted unbranched unsaturated C₁-C₃ alkylene. In embodiments, L^{2A} is independently an unsubstituted unbranched unsaturated C₁-C₂ alkylene.

L^{2C} is independently a bond or an unsubstituted alkylene (e.g., C₁-C₃₀, C₅-C₂₅, C₁₀-C₂₅, C₁₀-C₂₄, C₁-C₁₀, C₁-C₈, C₁-C₆, C₁-C₄, or C₁-C₂). In embodiments, L^{2C} is independently an unsubstituted C₁-C₃₀ alkylene. In embodiments, L^{2C} is independently an unsubstituted C₅-C₂₅ alkylene. In embodiments, L^{2C} is independently an unsubstituted C₁₀-C₂₅ alkylene. In embodiments, L^{2C} is independently an unsubstituted C₁₀-C₂₄ alkylene. In embodiments, L^{2C} is independently an unsubstituted C₁-C₁₀ alkylene. In embodiments, L^{2C} is independently an unsubstituted C₁-C₈ alkylene. In embodiments, L^{2C} is independently an unsubstituted C₁-C₆ alkylene. In embodiments, L^{2C} is independently an unsubstituted C₁-C₄ alkylene. In embodiments, L^{2C} is independently an unsubstituted C₁-C₃ alkylene. In embodiments, L^{2C} is independently an unsubstituted C₁-C₂ alkylene.

In embodiments, L^{2C} is independently an unsubstituted unbranched C₁-C₃₀ alkylene. In embodiments, L^{2C} is independently an unsubstituted unbranched C₅-C₂₅ alkylene. In embodiments, L^{2C} is independently an unsubstituted unbranched C₁₀-C₂₅ alkylene. In embodiments, L^{2C} is independently an unsubstituted unbranched C₁₀-C₂₄ alkylene. In embodiments, L^{2C} is independently an unsubstituted unbranched C₁-C₁₀ alkylene. In embodiments, L^{2C} is independently an unsubstituted unbranched C₁-C₈ alkylene. In embodiments, L^{2C} is independently an unsubstituted unbranched C₁-C₆ alkylene. In embodiments, L^{2C} is independently an unsubstituted unbranched C₁-C₄ alkylene. In embodiments, L^{2C} is independently an unsubstituted unbranched C₁-C₃ alkylene. In embodiments, L^{2C} is independently an unsubstituted unbranched C₁-C₂ alkylene.

In embodiments, L^{2C} is independently an unsubstituted unbranched saturated C₁-C₃₀ alkylene. In embodiments, L^{2C} is independently an unsubstituted unbranched saturated C₅-C₂₅ alkylene. In embodiments, L^{2C} is independently an unsubstituted unbranched saturated C₁₀-C₂₅ alkylene. In embodiments, L^{2C} is independently an unsubstituted unbranched saturated C₁₀-C₂₄ alkylene. In embodiments, L^{2C} is independently an unsubstituted unbranched saturated C₁-C₁₀ alkylene. In embodiments, L^{2C} is independently an unsubstituted unbranched saturated C₁-C₈ alkylene. In embodiments, L^{2C} is independently an unsubstituted unbranched saturated C₁-C₆ alkylene. In embodiments, L^{2C} is independently an unsubstituted unbranched saturated C₁-C₄ alkylene. In embodiments, L^{2C} is independently an unsubstituted unbranched saturated C₁-C₃ alkylene. In embodiments, L^{2C} is independently an unsubstituted unbranched saturated C₁-C₂ alkylene.

In embodiments, L^{2C} is independently an unsubstituted unbranched unsaturated C₁-C₃₀ alkylene. In embodiments, L^{2C} is independently an unsubstituted unbranched unsaturated C₅-C₂₅ alkylene. In embodiments, L^{2C} is independently an unsubstituted unbranched unsaturated C₁₀-C₂₅ alkylene. In embodiments, L^{2C} is independently an unsubstituted unbranched unsaturated C₁₀-C₂₄ alkylene. In embodiments, L^{2C} is independently an unsubstituted unbranched unsaturated C₁-C₁₀ alkylene. In embodiments, L^{2C} is independently an unsubstituted unbranched unsaturated C₁-C₈ alkylene. In embodiments, L^{2C} is independently an unsubstituted unbranched unsaturated C₁-C₆ alkylene. In embodiments, L^{2C} is independently an unsubstituted unbranched unsaturated C₁-C₄ alkylene. In embodiments, L^{2C} is independently an unsubstituted unbranched unsaturated C₁-C₃ alkylene. In embodiments, L^{2C} is independently an unsubstituted unbranched unsaturated C₁-C₂ alkylene.

L^{2D} is independently a bond or an unsubstituted alkylene (e.g., C₁-C₃₀, C₅-C₂₅, C₁₀-C₂₅, C₁₀-C₂₄, C₁-C₁₀, C₁-C₈, C₁-C₆, C₁-C₄, or C₁-C₂). In embodiments, L^{2D} is independently an unsubstituted C₁-C₃₀ alkylene. In embodiments, L^{2D} is independently an unsubstituted C₅-C₂₅ alkylene. In embodiments, L^{2D} is independently an unsubstituted C₁₀-C₂₅ alkylene. In embodiments, L^{2D} is independently an unsubstituted C₁₀-C₂₄ alkylene. In embodiments, L^{2D} is independently an unsubstituted C₁-C₁₀ alkylene. In embodiments, L^{2D} is independently an unsubstituted C₁-C₈ alkylene. In embodiments, L^{2D} is independently an unsubstituted C₁-C₆ alkylene. In embodiments, L^{2D} is independently an unsubstituted C₁-C₄ alkylene. In embodiments, L^{2D} is independently an unsubstituted C₁-C₃ alkylene. In embodiments, L^{2D} is independently an unsubstituted C₁-C₂ alkylene.

In embodiments, L^{2D} is independently an unsubstituted unbranched C₁-C₃₀ alkylene. In embodiments, L^{2D} is independently an unsubstituted unbranched C₅-C₂₅ alkylene. In embodiments, L^{2D} is independently an unsubstituted unbranched C₁₀-C₂₅ alkylene. In embodiments, L^{2D} is independently an unsubstituted unbranched C₁₀-C₂₄ alkylene. In embodiments, L^{2D} is independently an unsubstituted unbranched C₁-C₁₀ alkylene. In embodiments, L^{2D} is independently an unsubstituted unbranched C₁-C₈ alkylene. In embodiments, L^{2D} is independently an unsubstituted unbranched C₁-C₆ alkylene. In embodiments, L^{2D} is independently an unsubstituted unbranched C₁-C₄ alkylene. In embodiments, L^{2D} is independently an unsubstituted unbranched C₁-C₃ alkylene. In embodiments, L^{2D} is independently an unsubstituted unbranched C₁-C₂ alkylene.

In embodiments, L^{2D} is independently an unsubstituted unbranched saturated C₁-C₃₀ alkylene. In embodiments, L^{2D} is independently an unsubstituted unbranched saturated C₅-C₂₅ alkylene. In embodiments, L^{2D} is independently an unsubstituted unbranched saturated C₁₀-C₂₅ alkylene. In embodiments, L^{2D} is independently an unsubstituted unbranched saturated C₁₀-C₂₄ alkylene. In embodiments, L^{2D} is independently an unsubstituted unbranched saturated C₁-C₁₀ alkylene. In embodiments, L^{2D} is independently an unsubstituted unbranched saturated C₁-C₈ alkylene. In embodiments, L^{2D} is independently an unsubstituted unbranched saturated C₁-C₆ alkylene. In embodiments, L^{2D} is independently an unsubstituted unbranched saturated C₁-C₄ alkylene. In embodiments, L^{2D} is independently an unsubstituted unbranched saturated C₁-C₃ alkylene. In embodiments, L^{2D} is independently an unsubstituted unbranched saturated C₁-C₂ alkylene.

In embodiments, L^{2D} is independently an unsubstituted unbranched unsaturated C₁-C₃₀ alkylene. In embodiments, L^{2D} is independently an unsubstituted unbranched unsaturated C₅-C₂₅ alkylene. In embodiments, L^{2D} is independently an unsubstituted unbranched unsaturated C₁₀-C₂₅ alkylene. In embodiments, L^{2D} is independently an unsubstituted unbranched unsaturated C₁₀-C₂₄ alkylene. In embodiments, L^{2D} is independently an unsubstituted unbranched unsaturated C₁-C₁₀ alkylene. In embodiments, L^{2D} is independently an unsubstituted unbranched unsaturated C₁-C₈ alkylene. In embodiments, L^{2D} is independently an unsubstituted unbranched unsaturated C₁-C₆ alkylene. In embodiments, L^{2D} is independently an unsubstituted unbranched unsaturated C₁-C₄ alkylene. In embodiments, L^{2D} is independently an unsubstituted unbranched unsaturated C₁-C₃ alkylene. In embodiments, L^{2D} is independently an unsubstituted unbranched unsaturated C₁-C₂ alkylene.

L^{2E} is independently a bond or an unsubstituted alkylene (e.g., C₁-C₃₀, C₅-C₂₅, C₁₀-C₂₅, C₁₀-C₂₄, C₁-C₁₀, C₁-C₈, C₁-C₆, C₁-C₄, or C₁-C₂). In embodiments, L^{2E} is independently an unsubstituted C₁-C₃₀ alkylene. In embodiments, L^{2E} is independently an unsubstituted C₅-C₂₅ alkylene. In embodiments, L^{2E} is independently an unsubstituted C₁₀-C₂₅ alkylene. In embodiments, L^{2E} is independently an unsubstituted C₁₀-C₂₄ alkylene. In embodiments, L^{2E} is independently an unsubstituted C₁-C₁₀ alkylene. In embodiments, L^{2E} is independently an unsubstituted C₁-C₈ alkylene. In embodiments, L^{2E} is independently an unsubstituted C₁-C₆ alkylene. In embodiments, L^{2E} is independently an unsubstituted C₁-C₄ alkylene. In embodiments, L^{2E} is independently an unsubstituted C₁-C₃ alkylene. In embodiments, L^{2E} is independently an unsubstituted C₁-C₂ alkylene.

In embodiments, L^{2E} is independently an unsubstituted unbranched C₁-C₃₀ alkylene. In embodiments, L^{2E} is independently an unsubstituted unbranched C₅-C₂₅ alkylene. In embodiments, L^{2E} is independently an unsubstituted unbranched C₁₀-C₂₅ alkylene. In embodiments, L^{2E} is independently an unsubstituted unbranched C₁₀-C₂₄ alkylene. In embodiments, L^{2E} is independently an unsubstituted unbranched C₁-C₁₀ alkylene. In embodiments, L^{2E} is independently an unsubstituted unbranched C₁-C₈ alkylene. In embodiments, L^{2E} is independently an unsubstituted unbranched C₁-C₆ alkylene. In embodiments, L^{2E} is independently an unsubstituted unbranched C₁-C₄ alkylene. In embodiments, L^{2E} is independently an unsubstituted unbranched C₁-C₃ alkylene. In embodiments, L^{2E} is independently an unsubstituted unbranched C₁-C₂ alkylene.

In embodiments, L^{2E} is independently an unsubstituted unbranched saturated C₁-C₃₀ alkylene. In embodiments, L^{2E} is independently an unsubstituted unbranched saturated C₅-C₂₅ alkylene. In embodiments, L^{2E} is independently an unsubstituted unbranched saturated C₁₀-C₂₅ alkylene. In embodiments, L^{2E} is independently an unsubstituted unbranched saturated C₁₀-C₂₄ alkylene. In embodiments, L^{2E} is independently an unsubstituted unbranched saturated C₁-C₁₀ alkylene. In embodiments, L^{2E} is independently an unsubstituted unbranched saturated C₁-C₈ alkylene. In embodiments, L^{2E} is independently an unsubstituted unbranched saturated C₁-C₆ alkylene. In embodiments, L^{2E} is independently an unsubstituted unbranched saturated C₁-C₄ alkylene. In embodiments, L^{2E} is independently an unsubstituted unbranched saturated C₁-C₃ alkylene. In embodiments, L^{2E} is independently an unsubstituted unbranched saturated C₁-C₂ alkylene.

In embodiments, L^{2E} is independently an unsubstituted unbranched unsaturated C₁-C₃₀ alkylene. In embodiments, L^{2E} is independently an unsubstituted unbranched unsaturated C₅-C₂₅ alkylene. In embodiments, L^{2E} is independently an unsubstituted unbranched unsaturated C₁₀-C₂₅ alkylene. In embodiments, L^{2E} is independently an unsubstituted unbranched unsaturated C₁₀-C₂₄ alkylene. In embodiments, L^{2E} is independently an unsubstituted unbranched unsaturated C₁-C₁₀ alkylene. In embodiments, L^{2E} is independently an unsubstituted unbranched unsaturated C₁-C₈ alkylene. In embodiments, L^{2E} is independently an unsubstituted unbranched unsaturated C₁-C₆ alkylene. In embodiments, L^{2E} is independently an unsubstituted unbranched unsaturated C₁-C₄ alkylene. In embodiments, L^{2E} is independently an unsubstituted unbranched unsaturated C₁-C₃ alkylene. In embodiments, L^{2E} is independently an unsubstituted unbranched unsaturated C₁-C₂ alkylene.

L³ is independently a bond, a bond, —N(R²³)—, —O—, —S—, —C(O)—, —N(R²³)C(O)—, —C(O)N(R²⁴)—, —N(R²³)C(O)N(R²⁴)—, —C(O)O—, —OC(O)—, —N(R²³)C(O)O—, —OC(O)N(R²⁴)—, —OPO₂—O—, —O—P(O)(S)—O—, —O—P(O)(R²⁵)—O—, —O—P(S)(R²⁵)—O—, —O—P(O)(NR²³R²⁴)—N—, —O—P(S)(NR²³R²⁴)—N—, —O—P(O)(NR²³R²⁴)—O—, —O—P(S)(NR²³R²⁴)—O—, —P(O)(NR²³R²⁴)—N—, —P(S)(NR²³R²⁴)—N—, —P(O)(NR²³R²⁴)—O—, —P(S)(NR²³R²⁴)—O—, —S—S—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L³ is independently a bond, a

bond, —N(R²³)—, —O—, —S—, —C(O)—, —N(R²³)C(O)—, —C(O)N(R²⁴)—, —N(R²³)C(O)N(R²⁴)—, —C(O)O—, —OC(O)—, —N(R²³)C(O)O—, —OC(O)N(R²⁴)—, —OPO₂—O—, —O—P(O)(S)—O—, —O—P(O)(R²⁵)—O—, —O—P(S)(R²⁵)—O—, —O—P(O)(NR²³R²⁴)—N—, —O—P(S)(NR²³R²⁴)—N—, —O—P(O)(NR²³R²⁴)—O—, —O—P(S)(NR²³R²⁴)—O—, —P(O)(NR²³R²⁴)—N—, —P(S)(NR²³R²⁴)—N—, —P(O)(NR²³R²⁴)—O—, —P(S)(NR²³R²⁴)—O—, —S—S—, substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L³ is independently a bond, a bond, —N(R²³)—, —O—, —S—, —C(O)—, —N(R²³)C(O)—, —C(O)N(R²⁴)—, —N(R²³)C(O)N(R²⁴)—, —C(O)O—, —OC(O)—, —N(R²³)C(O)O—, —OC(O)N(R²⁴)—, —OPO₂—O—, —O—P(O)(S)—O—, —O—P(O)(R²⁵)—O—, —O—P(S)(R²⁵)—O—, —O—P(O)(NR²³R²⁴)—N—, —O—P(S)(NR²³R²⁴)—N—, —O—P(O)(NR²³R²⁴)—O—, —O—P(S)(NR²³R²⁴)—O—, —P(O)(NR²³R²⁴)—N—, —P(S)(NR²³R²⁴)—N—, —P(O)(NR²³R²⁴)—O—, —P(S)(NR²³R²⁴)—O—, —S—S—, unsubstituted alkylene (e.g., C₁-C₂₀, C₁-C₁₂, C₁-C₈, C₁-C₆, C₁-C₄, or C₁-C₂), unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L³ is substituted, L³ is substituted with a substituent group. In embodiments, when L³ is substituted, L³ is substituted with a size-limited substituent group. In embodiments, when L³ is substituted, L³ is substituted with a lower substituent group.

L⁴ is independently a bond, a bond, —N(R²³)—, —O—, —S—, —C(O)—, —N(R²³)C(O)—, —C(O)N(R²⁴)—, —N(R²³)C(O)N(R²⁴)—, —C(O)O—, —OC(O)—, —N(R²³)C(O)O—, —OC(O)N(R²⁴)—, —OPO₂—O—, —O—P(O)(S)—O—, —O—P(O)(R²⁵)—O—, —O—P(S)(R²⁵)—O—, —O—P(O)(NR²³R²⁴)—N—, —O—P(S)(NR²³R²⁴)—N—, —O—P(O)(NR²³R²⁴)—O—, —O—P(S)(NR²³R²⁴)—O—, —P(O)(NR²³R²⁴)—N—, —P(S)(NR²³R²⁴)—N—, —P(O)(NR²³R²⁴)—O—, —P(S)(NR²³R²⁴)—O—, —S—S—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L⁴ is a bond, a bond, —N(R²³)—, —O—, —S—, —C(O)—, —N(R²³)C(O)—, —C(O)N(R²⁴)—, —N(R²³)C(O)N(R²⁴)—, —C(O)O—, —OC(O)—, —N(R²³)C(O)O—, —OC(O)N(R²⁴)—, —OPO₂—O—, —O—P(O)(S)—O—, —O—P(O)(R²⁵)—O—, —O—P(S)(R²⁵)—O—, —O—P(O)(NR²³R²⁴)—N—, —O—P(S)(NR²³R²⁴)—N—, —O—P(O)(NR²³R²⁴)—O—, —O—P(S)(NR²³R²⁴)—O—, —P(O)(NR²³R²⁴)—N—, —P(S)(NR²³R²⁴)—N—, —P(O)(NR²³R²⁴)—O—, —P(S)(NR²³R²⁴)—O—, —S—S—, substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L⁴ is a bond, a bond, —N(R²³)—, —O—, —S—, —C(O)—, —N(R²³)C(O)—, —C(O)N(R²⁴)—, —N(R²³)C(O)N(R²⁴)—, —C(O)O—, —OC(O)—, —N(R²³)C(O)O—, —OC(O)N(R²⁴)—, —OPO₂—O—, —O—P(O)(S)—O—, —O—P(O)(R²⁵)—O—, —O—P(S)(R²⁵)—O—, —O—P(O)(NR²³R²⁴)—N—, —O—P(S)(NR²³R²⁴)—N—, —O—P(O)(NR²³R²⁴)—O—, —O—P(S)(NR²³R²⁴)—O—, —P(O)(NR²³R²⁴)—N—, —P(S)(NR²³R²⁴)—N—, —P(O)(NR²³R²⁴)—O—, —P(S)(NR²³R²⁴)—O—, —S—S—, unsubstituted alkylene (e.g., C₁-C₂₀, C₁-C₁₂, C₁-C₈, C₁-C₆, C₁-C₄, or C₁-C₂), unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L⁴ is substituted, L⁴ is substituted with a substituent group. In embodiments, when L⁴ is substituted, L⁴ is substituted with a size-limited substituent group. In embodiments, when L⁴ is substituted, L⁴ is substituted with a lower substituent group.

R²³ is independently hydrogen or unsubstituted alkyl (e.g., C₁-C₂₃, C₁-C₁₂, C₁-C₈, C₁-C₆, C₁-C₄, or C₁-C₂). In embodiments, R²³ is independently hydrogen. In embodiments, R²³ is independently unsubstituted C₁-C₂₃ alkyl. In embodiments, R²³ is independently hydrogen or unsubstituted C₁-C₁₂ alkyl. In embodiments, R²³ is independently hydrogen or unsubstituted C₁-C₁₀ alkyl. In embodiments, R²³ is independently hydrogen or unsubstituted C₁-C₈ alkyl. In embodiments, R²³ is independently hydrogen or unsubstituted C₁-C₆ alkyl. In embodiments, R²³ is independently hydrogen or unsubstituted C₁-C₄ alkyl. In embodiments, R²³ is independently hydrogen or unsubstituted C₁-C₂ alkyl.

R²⁴ is independently hydrogen or unsubstituted alkyl (e.g., C₁-C₂₃, C₁-C₁₂, C₁-C₈, C₁-C₆, C₁-C₄, or C₁-C₂). In embodiments, R²⁴ is independently hydrogen. In embodiments, R²⁴ is independently unsubstituted C₁-C₂₃ alkyl. In embodiments, R²⁴ is independently hydrogen or unsubstituted C₁-C₁₂ alkyl. In embodiments, R²⁴ is independently hydrogen or unsubstituted C₁-C₁₀ alkyl. In embodiments, R²⁴ is independently hydrogen or unsubstituted C₁-C₈ alkyl. In embodiments, R²⁴ is independently hydrogen or unsubstituted C₁-C₆ alkyl. In embodiments, R²⁴ is independently hydrogen or unsubstituted C₁-C₄ alkyl. In embodiments, R²⁴ is independently hydrogen or unsubstituted C₁-C₂ alkyl.

R²⁵ is independently hydrogen or unsubstituted alkyl (e.g., C₁-C₂₃, C₁-C₁₂, C₁-C₈, C₁-C₆, C₁-C₄, or C₁-C₂). In embodiments, R²⁵ is independently hydrogen. In embodiments, R²⁵ is independently unsubstituted C₁-C₂₃ alkyl. In embodiments, R²⁵ is independently hydrogen or unsubstituted C₁-C₁₂ alkyl. In embodiments, R²⁵ is independently hydrogen or unsubstituted C₁-C₁₀ alkyl. In embodiments, R²⁵ is independently hydrogen or unsubstituted C₁-C₈ alkyl. In embodiments, R²⁵ is independently hydrogen or unsubstituted C₁-C₆ alkyl. In embodiments, R²⁵ is independently hydrogen or unsubstituted C₁-C₄ alkyl. In embodiments, R²⁵ is independently hydrogen or unsubstituted C₁-C₂ alkyl.

L⁵ is independently a

bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L⁵ is independently a
bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L⁵ is independently a bond, —NH—, —O—, —S—, —C(O)—, —NHC(O)—, —NHC(O)NH—, —C(O)O—, —OC(O)—, —C(O)NH—, unsubstituted alkylene (e.g., C₁-C₂₀, C₁-C₁₂, C₁-C₈, C₁-C₆, C₁-C₄, or C₁-C₂), unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L⁵ is substituted, L⁵ is substituted with a substituent group. In embodiments, when L⁵ is substituted, L⁵ is substituted with a size-limited substituent group. In embodiments, when L⁵ is substituted, L⁵ is substituted with a lower substituent group.

L^{5A} is a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L^{5A} is a

bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L^{5A} is a
bond, —NH—, —O—, —S—, —C(O)—, —NHC(O)—, —NHC(O)NH—, —C(O)O—, —OC(O)—, —C(O)NH—, unsubstituted alkylene (e.g., C₁-C₂₀, C₁-C₁₂, C₁-C₈, C₁-C₆, C₁-C₄, or C₁-C₂), unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L^{5A} is substituted, L^{5A} is substituted with a substituent group. In embodiments, when L^{5A} is substituted, L^{5A} is substituted with a size-limited substituent group. In embodiments, when L^{5A} is substituted, L^{5A} is substituted with a lower substituent group.

L^{5B} is a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L^{5B} is a

bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L^{5B} is a
bond, —NH—, —O—, —S—, —C(O)—, —NHC(O)—, —NHC(O)NH—, —C(O)O—, —OC(O)—, —C(O)NH—, unsubstituted alkylene (e.g., C₁-C₂₀, C₁-C₁₂, C₁-C₈, C₁-C₆, C₁-C₄, or C₁-C₂), unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L^{5B} is substituted, L^{5B} is substituted with a substituent group. In embodiments, when L^{5B} is substituted, L^{5B} is substituted with a size-limited substituent group. In embodiments, when L^{5B} is substituted, L^{5B} is substituted with a lower substituent group.

L^{5C} is a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L^{5C} is a

bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L^{5C} is a
bond, —NH—, —O—, —S—, —C(O)—, —NHC(O)—, —NHC(O)NH—, —C(O)O—, —OC(O)—, —C(O)NH—, unsubstituted alkylene (e.g., C₁-C₂₀, C₁-C₁₂, C₁-C₈, C₁-C₆, C₁-C₄, or C₁-C₂), unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L^{5C} is substituted, L^{5C} is substituted with a substituent group. In embodiments, when L^{5C} is substituted, L^{5C} is substituted with a size-limited substituent group. In embodiments, when L^{5C} is substituted, L^{5C} is substituted with a lower substituent group.

L^{5D} is a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L^{5D} is a

bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L^{5D} is a
bond, —NH—, —O—, —S—, —C(O)—, —NHC(O)—, —NHC(O)NH—, —C(O)O—, —OC(O)—, —C(O)NH—, unsubstituted alkylene (e.g., C₁-C₂₀, C₁-C₁₂, C₁-C₈, C₁-C₆, C₁-C₄, or C₁-C₂), unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L^{5D} is substituted, L^{5D} is substituted with a substituent group. In embodiments, when L^{5D} is substituted, L^{5D} is substituted with a size-limited substituent group. In embodiments, when L^{5D} is substituted, L^{5D} is substituted with a lower substituent group.

L^{5E} is a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L^{5E} is a

bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L^{5E} is a
bond, —NH—, —O—, —S—, —C(O)—, —NHC(O)—, —NHC(O)NH—, —C(O)O—, —OC(O)—, —C(O)NH—, unsubstituted alkylene (e.g., C₁-C₂₀, C₁-C₁₂, C₁-C₈, C₁-C₆, C₁-C₄, or C₁-C₂), unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L^{5E} is substituted, L^{5E} is substituted with a substituent group. In embodiments, when L^{5E} is substituted, L^{5E} is substituted with a size-limited substituent group. In embodiments, when L^{5E} is substituted, L^{5E} is substituted with a lower substituent group.

L⁶ is independently a

bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L⁶ is independently a
bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L⁶ is independently a bond, —NH—, —O—, —S—, —C(O)—, —NHC(O)—, —NHC(O)NH—, —C(O)O—, —OC(O)—, —C(O)NH—, unsubstituted alkylene (e.g., C₁-C₂₀, C₁-C₁₂, C₁-C₈, C₁-C₆, C₁-C₄, or C₁-C₂), unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L⁶ is substituted, L⁶ is substituted with a substituent group. In embodiments, when L⁶ is substituted, L⁶ is substituted with a size-limited substituent group. In embodiments, when L⁶ is substituted, L⁶ is substituted with a lower substituent group.

L^{6A} is a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L^{6A} is a

bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L^{6A} is a
bond, —NH—, —O—, —S—, —C(O)—, —NHC(O)—, —NHC(O)NH—, —C(O)O—, —OC(O)—, —C(O)NH—, unsubstituted alkylene (e.g., C₁-C₂₀, C₁-C₁₂, C₁-C₈, C₁-C₆, C₁-C₄, or C₁-C₂), unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L^{6A} is substituted, L^{6A} is substituted with a substituent group. In embodiments, when L^{6A} is substituted, L^{6A} is substituted with a size-limited substituent group. In embodiments, when L^{6A} is substituted, L^{6A} is substituted with a lower substituent group.

L^{6B} is a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L^{6B} is a

bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L^{6B} is a
bond, —NH—, —O—, —S—, —C(O)—, —NHC(O)—, —NHC(O)NH—, —C(O)O—, —OC(O)—, —C(O)NH—, unsubstituted alkylene (e.g., C₁-C₂₀, C₁-C₁₂, C₁-C₈, C₁-C₆, C₁-C₄, or C₁-C₂), unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L^{6B} is substituted, L^{6B} is substituted with a substituent group. In embodiments, when L^{6B} is substituted, L^{6B} is substituted with a size-limited substituent group. In embodiments, when L^{6B} is substituted, L^{6B} is substituted with a lower substituent group.

L^{6C} is a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L^{6C} is a

bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L^{6C} is a
bond, —NH—, —O—, —S—, —C(O)—, —NHC(O)—, —NHC(O)NH—, —C(O)O—, —OC(O)—, —C(O)NH—, unsubstituted alkylene (e.g., C₁-C₂₀, C₁-C₁₂, C₁-C₈, C₁-C₆, C₁-C₄, or C₁-C₂), unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L^{6C} is substituted, L^{6C} is substituted with a substituent group. In embodiments, when L^{6C} is substituted, L^{6C} is substituted with a size-limited substituent group. In embodiments, when L^{6C} is substituted, L^{6C} is substituted with a lower substituent group.

L^{6D} is a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L^{6D} is a

bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L^{6D} is a
bond, —NH—, —O—, —S—, —C(O)—, —NHC(O)—, —NHC(O)NH—, —C(O)O—, —OC(O)—, —C(O)NH—, unsubstituted alkylene (e.g., C₁-C₂₀, C₁-C₁₂, C₁-C₈, C₁-C₆, C₁-C₄, or C₁-C₂), unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L^{6D} is substituted, L^{6D} is substituted with a substituent group. In embodiments, when L^{6D} is substituted, L^{6D} is substituted with a size-limited substituent group. In embodiments, when L^{6D} is substituted, L^{6D} is substituted with a lower substituent group.

L^{6E} is a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L^{6E} is a

bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L^{6E} is a
bond, —NH—, —O—, —S—, —C(O)—, —NHC(O)—, —NHC(O)NH—, —C(O)O—, —OC(O)—, —C(O)NH—, unsubstituted alkylene (e.g., C₁-C₂₀, C₁-C₁₂, C₁-C₈, C₁-C₆, C₁-C₄, or C₁-C₂), unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L^{6E} is substituted, L^{6E} is substituted with a substituent group. In embodiments, when L^{6E} is substituted, L^{6E} is substituted with a size-limited substituent group. In embodiments, when L^{6E} is substituted, L^{6E} is substituted with a lower substituent group.

In embodiments, L⁷ is independently substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂). In embodiments, L⁷ is independently substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂). In embodiments, L⁷ is independently unsubstituted alkylene (e.g., C₁-C₂₀, C₁-C₁₂, C₁-C₈, C₁-C₆, C₁-C₄, or C₁-C₂).

In embodiments, L⁷ is independently substituted (e.g., substituted with a substituent group, a size-limited substituent group, or lower substituent group) or unsubstituted heteroalkylene (e.g., 2 to 20 membered, 2 to 12 membered, 2 to 10 membered, 2 to 8 membered, 2 to 6 membered, or 2 to 4 membered). In embodiments, L⁷ is independently substituted (e.g., substituted with a substituent group, a size-limited substituent group, or lower substituent group) heteroalkylene (e.g., 2 to 20 membered, 2 to 12 membered, 2 to 10 membered, 2 to 8 membered, 2 to 6 membered, or 2 to 4 membered). In embodiments, L⁷ is independently unsubstituted heteroalkylene (e.g., 2 to 20 membered, 2 to 12 membered, 2 to 10 membered, 2 to 8 membered, 2 to 6 membered, or 2 to 4 membered). In embodiments, L⁷ is independently substituted (e.g., substituted with a substituent group, a size-limited substituent group, or lower substituent group) or unsubstituted heteroalkenylene (e.g., 2 to 20 membered, 2 to 12 membered, 2 to 10 membered, 2 to 8 membered, 2 to 6 membered, or 2 to 4 membered). In embodiments, L⁷ is independently substituted (e.g., substituted with a substituent group, a size-limited substituent group, or lower substituent group) heteroalkenylene (e.g., 2 to 20 membered, 2 to 12 membered, 2 to 10 membered, 2 to 8 membered, 2 to 6 membered, or 2 to 4 membered). In embodiments, L⁷ is independently unsubstituted heteroalkenylene (e.g., 2 to 20 membered, 2 to 12 membered, 2 to 10 membered, 2 to 8 membered, 2 to 6 membered, or 2 to 4 membered). In embodiments, when L⁷ is substituted, L⁷ is substituted with a substituent group. In embodiments, when L⁷ is substituted, L⁷ is substituted with a size-limited substituent group. In embodiments, when L⁷ is substituted, L⁷ is substituted with a lower substituent group.

L^{8C} is independently a bond, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), or substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L^{8C} is independently a bond, or substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L^{8C} is independently a bond, unsubstituted alkylene (e.g., C₁-C₂₀, C₁-C₁₂, C₁-C₈, C₁-C₆, C₁-C₄, or C₁-C₂), or unsubstituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, when L^{8C} is substituted, L^{8C} is substituted with a substituent group. In embodiments, when L^{8C} is substituted, L^{8C} is substituted with a size-limited substituent group. In embodiments, when L^{8C} is substituted, L^{8C} is substituted with a lower substituent group.

L^{8D} is independently a bond, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), or substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L^{8D} is independently a bond, or substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L^{8D} is independently a bond, unsubstituted alkylene (e.g., C₁-C₂₀, C₁-C₁₂, C₁-C₈, C₁-C₆, C₁-C₄, or C₁-C₂), or unsubstituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, when L^{8D} is substituted, L^{8D} is substituted with a substituent group. In embodiments, when L^{8D} is substituted, L^{8D} is substituted with a size-limited substituent group. In embodiments, when L^{8D} is substituted, L^{8D} is substituted with a lower substituent group.

L^{8E} is independently a bond, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), or substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L^{8E} is independently a bond, or substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L^{8E} is independently a bond, unsubstituted alkylene (e.g., C₁-C₂₀, C₁-C₁₂, C₁-C₈, C₁-C₆, C₁-C₄, or C₁-C₂), or unsubstituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, when L^{8E} is substituted, L^{8E} is substituted with a substituent group. In embodiments, when L^{8E} is substituted, L^{8E} is substituted with a size-limited substituent group. In embodiments, when L^{8E} is substituted, L^{8E} is substituted with a lower substituent group.

In embodiments, L¹⁰ is independently substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂). In embodiments, L¹⁰ is independently substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂). In embodiments, L¹⁰ is independently unsubstituted alkylene (e.g., C₁-C₂₀, C₁-C₁₂, C₁-C₈, C₁-C₆, C₁-C₄, or C₁-C₂).

In embodiments, L¹⁰ is independently substituted (e.g., substituted with a substituent group, a size-limited substituent group, or lower substituent group) or unsubstituted heteroalkylene (e.g., 2 to 20 membered, 2 to 12 membered, 2 to 10 membered, 2 to 8 membered, 2 to 6 membered, or 2 to 4 membered). In embodiments, L¹⁰ is independently substituted (e.g., substituted with a substituent group, a size-limited substituent group, or lower substituent group) heteroalkylene (e.g., 2 to 20 membered, 2 to 12 membered, 2 to 10 membered, 2 to 8 membered, 2 to 6 membered, or 2 to 4 membered). In embodiments, L¹⁰ is independently unsubstituted heteroalkylene (e.g., 2 to 20 membered, 2 to 12 membered, 2 to 10 membered, 2 to 8 membered, 2 to 6 membered, or 2 to 4 membered). In embodiments, L¹⁰ is independently substituted (e.g., substituted with a substituent group, a size-limited substituent group, or lower substituent group) or unsubstituted heteroalkenylene (e.g., 2 to 20 membered, 2 to 12 membered, 2 to 10 membered, 2 to 8 membered, 2 to 6 membered, or 2 to 4 membered). In embodiments, L¹⁰ is independently substituted (e.g., substituted with a substituent group, a size-limited substituent group, or lower substituent group) heteroalkenylene (e.g., 2 to 20 membered, 2 to 12 membered, 2 to 10 membered, 2 to 8 membered, 2 to 6 membered, or 2 to 4 membered). In embodiments, L¹⁰ is independently unsubstituted heteroalkenylene (e.g., 2 to 20 membered, 2 to 12 membered, 2 to 10 membered, 2 to 8 membered, 2 to 6 membered, or 2 to 4 membered). In embodiments, when L¹⁰ is substituted, L¹⁰ is substituted with a substituent group. In embodiments, when L¹⁰ is substituted, L¹⁰ is substituted with a size-limited substituent group. In embodiments, when L¹⁰ is substituted, L¹⁰ is substituted with a lower substituent group.

In embodiments, R¹ is unsubstituted alkyl (e.g., C₁-C₂₅, C₁-C₂₀, C₁-C₁₂, C₁-C₈, C₁-C₆, C₁-C₄, or C₁-C₂). In embodiments, R¹ is unsubstituted C₁-C₂₅ alkyl. In embodiments, R¹ is unsubstituted C₁-C₂₀ alkyl. In embodiments, R¹ is unsubstituted C₁-C₁₂ alkyl. In embodiments, R¹ is unsubstituted C₁-C₈ alkyl. In embodiments, R¹ is unsubstituted C₁-C₆ alkyl. In embodiments, R¹ is unsubstituted C₁-C₄ alkyl. In embodiments, R¹ is unsubstituted C₁-C₂ alkyl.

In embodiments, R¹ is unsubstituted branched alkyl (e.g., C₁-C₂₅, C₁-C₂₀, C₁-C₁₂, C₁-C₈, C₁-C₆, C₁-C₄, or C₁-C₂). In embodiments, R¹ is unsubstituted branched C₁-C₂₅ alkyl. In embodiments, R¹ is unsubstituted branched C₁-C₂₀ alkyl. In embodiments, R¹ is unsubstituted branched C₁-C₁₂ alkyl. In embodiments, R¹ is unsubstituted branched C₁-C₈ alkyl. In embodiments, R¹ is unsubstituted branched C₁-C₆ alkyl. In embodiments, R¹ is unsubstituted branched C₁-C₄ alkyl. In embodiments, R¹ is unsubstituted branched C₁-C₂ alkyl.

In embodiments, R¹ is unsubstituted unbranched alkyl (e.g., C₁-C₂₅, C₁-C₂₀, C₁-C₁₂, C₁-C₈, C₁-C₆, C₁-C₄, or C₁-C₂). In embodiments, R¹ is unsubstituted unbranched C₁-C₂₅ alkyl. In embodiments, R¹ is unsubstituted unbranched C₁-C₂₀ alkyl. In embodiments, R¹ is unsubstituted unbranched C₁-C₁₂ alkyl. In embodiments, R¹ is unsubstituted unbranched C₁-C₈ alkyl. In embodiments, R¹ is unsubstituted unbranched C₁-C₆ alkyl. In embodiments, R¹ is unsubstituted unbranched C₁-C₄ alkyl. In embodiments, R¹ is unsubstituted unbranched C₁-C₂ alkyl.

In embodiments, R¹ is unsubstituted branched saturated alkyl (e.g., C₁-C₂₅, C₁-C₂₀, C₁-C₁₂, C₁-C₈, C₁-C₆, C₁-C₄, or C₁-C₂). In embodiments, R¹ is unsubstituted branched saturated C₁-C₂₅ alkyl. In embodiments, R¹ is unsubstituted branched saturated C₁-C₂₀ alkyl. In embodiments, R¹ is unsubstituted branched saturated C₁-C₁₂ alkyl. In embodiments, R¹ is unsubstituted branched saturated C₁-C₈ alkyl. In embodiments, R¹ is unsubstituted branched saturated C₁-C₆ alkyl. In embodiments, R¹ is unsubstituted branched saturated C₁-C₄ alkyl. In embodiments, R¹ is unsubstituted branched saturated C₁-C₂ alkyl.

In embodiments, R¹ is unsubstituted branched unsaturated alkyl (e.g., C₁-C₂₅, C₁-C₂₀, C₁-C₁₂, C₁-C₈, C₁-C₆, C₁-C₄, or C₁-C₂). In embodiments, R¹ is unsubstituted branched unsaturated C₁-C₂₅ alkyl. In embodiments, R¹ is unsubstituted branched unsaturated C₁-C₂₀ alkyl. In embodiments, R¹ is unsubstituted branched unsaturated C₁-C₁₂ alkyl. In embodiments, R¹ is unsubstituted branched unsaturated C₁-C₈ alkyl. In embodiments, R¹ is unsubstituted branched unsaturated C₁-C₆ alkyl. In embodiments, R¹ is unsubstituted branched unsaturated C₁-C₄ alkyl. In embodiments, R¹ is unsubstituted branched saturated C₁-C₂ alkyl.

In embodiments, R¹ is unsubstituted unbranched saturated alkyl (e.g., C₁-C₂₅, C₁-C₂₀, C₁-C₁₂, C₁-C₈, C₁-C₆, C₁-C₄, or C₁-C₂). In embodiments, R¹ is unsubstituted unbranched saturated C₁-C₂₅ alkyl. In embodiments, R¹ is unsubstituted unbranched saturated C₁-C₂₀ alkyl. In embodiments, R¹ is unsubstituted unbranched saturated C₁-C₁₂ alkyl. In embodiments, R¹ is unsubstituted unbranched saturated C₁-C₈ alkyl. In embodiments, R¹ is unsubstituted unbranched saturated C₁-C₆ alkyl. In embodiments, R¹ is unsubstituted unbranched saturated C₁-C₄alkyl. In embodiments, R¹ is unsubstituted unbranched saturated C₁-C₂ alkyl.

In embodiments, R¹ is unsubstituted unbranched unsaturated alkyl (e.g., C₁-C₂₅, C₁-C₂₀, C₁-C₁₂, C₁-C₈, C₁-C₆, C₁-C₄, or C₁-C₂). In embodiments, R¹ is unsubstituted unbranched unsaturated C₁-C₂₅ alkyl. In embodiments, R¹ is unsubstituted unbranched unsaturated C₁-C₂₀ alkyl. In embodiments, R¹ is unsubstituted unbranched unsaturated C₁-C₁₂ alkyl. In embodiments, R¹ is unsubstituted unbranched unsaturated C₁-C₈ alkyl. In embodiments, R¹ is unsubstituted unbranched unsaturated C₁-C₆ alkyl. In embodiments, R¹ is unsubstituted unbranched unsaturated C₁-C₄ alkyl. In embodiments, R¹ is unsubstituted unbranched unsaturated C₁-C₂ alkyl.

In embodiments, R¹ is unsubstituted C₉-C₁₉ alkyl. In embodiments, R¹ is unsubstituted branched C₉-C₁₉ alkyl. In embodiments, R¹ is unsubstituted unbranched C₉-C₁₉ alkyl. In embodiments, R¹ is unsubstituted branched saturated C₉-C₁₉ alkyl. In embodiments, R¹ is unsubstituted branched unsaturated C₉-C₁₉ alkyl. In embodiments, R¹ is unsubstituted unbranched saturated C₉-C₁₉ alkyl. In embodiments, R¹ is unsubstituted unbranched unsaturated C₉-C₁₉ alkyl.

In embodiments, R² is unsubstituted alkyl (e.g., C₁-C₂₅, C₁-C₂₀, C₁-C₁₂, C₁-C₈, C₁-C₆, C₁-C₄, or C₁-C₂). In embodiments, R² is unsubstituted C₁-C₂₅ alkyl. In embodiments, R² is unsubstituted C₁-C₂₀ alkyl. In embodiments, R² is unsubstituted C₁-C₁₂ alkyl. In embodiments, R² is unsubstituted C₁-C₈ alkyl. In embodiments, R² is unsubstituted C₁-C₆ alkyl. In embodiments, R² is unsubstituted C₁-C₄ alkyl. In embodiments, R² is unsubstituted C₁-C₂ alkyl.

In embodiments, R² is unsubstituted branched alkyl (e.g., C₁-C₂₅, C₁-C₂₀, C₁-C₁₂, C₁-C₈, C₁-C₆, C₁-C₄, or C₁-C₂). In embodiments, R² is unsubstituted branched C₁-C₂₅ alkyl. In embodiments, R² is unsubstituted branched C₁-C₂₀ alkyl. In embodiments, R² is unsubstituted branched C₁-C₁₂ alkyl. In embodiments, R² is unsubstituted branched C₁-C₈ alkyl. In embodiments, R² is unsubstituted branched C₁-C₆ alkyl. In embodiments, R² is unsubstituted branched C₁-C₄ alkyl. In embodiments, R² is unsubstituted branched C₁-C₂ alkyl.

In embodiments, R² is unsubstituted unbranched alkyl (e.g., C₁-C₂₅, C₁-C₂₀, C₁-C₁₂, C₁-C₈, C₁-C₆, C₁-C₄, or C₁-C₂). In embodiments, R² is unsubstituted unbranched C₁-C₂₅ alkyl. In embodiments, R² is unsubstituted unbranched C₁-C₂₀ alkyl. In embodiments, R² is unsubstituted unbranched C₁-C₁₂ alkyl. In embodiments, R² is unsubstituted unbranched C₁-C₈ alkyl. In embodiments, R² is unsubstituted unbranched C₁-C₆ alkyl. In embodiments, R² is unsubstituted unbranched C₁-C₄ alkyl. In embodiments, R² is unsubstituted unbranched C₁-C₂ alkyl.

In embodiments, R² is unsubstituted branched saturated alkyl (e.g., C₁-C₂₅, C₁-C₂₀, C₁-C₁₂, C₁-C₈, C₁-C₆, C₁-C₄, or C₁-C₂). In embodiments, R² is unsubstituted branched saturated C₁-C₂₅ alkyl. In embodiments, R² is unsubstituted branched saturated C₁-C₂₀ alkyl. In embodiments, R² is unsubstituted branched saturated C₁-C₁₂ alkyl. In embodiments, R² is unsubstituted branched saturated C₁-C₈ alkyl. In embodiments, R² is unsubstituted branched saturated C₁-C₆ alkyl. In embodiments, R² is unsubstituted branched saturated C₁-C₄ alkyl. In embodiments, R² is unsubstituted branched saturated C₁-C₂ alkyl.

In embodiments, R² is unsubstituted branched unsaturated alkyl (e.g., C₁-C₂₅, C₁-C₂₀, C₁-C₁₂, C₁-C₈, C₁-C₆, C₁-C₄, or C₁-C₂). In embodiments, R² is unsubstituted branched unsaturated C₁-C₂₅ alkyl. In embodiments, R² is unsubstituted branched unsaturated C₁-C₂₀ alkyl. In embodiments, R² is unsubstituted branched unsaturated C₁-C₁₂ alkyl. In embodiments, R² is unsubstituted branched unsaturated C₁-C₈ alkyl. In embodiments, R² is unsubstituted branched unsaturated C₁-C₆ alkyl. In embodiments, R² is unsubstituted branched unsaturated C₁-C₄ alkyl. In embodiments, R² is unsubstituted branched saturated C₁-C₂ alkyl.

In embodiments, R² is unsubstituted unbranched saturated alkyl (e.g., C₁-C₂₅, C₁-C₂₀, C₁-C₁₂, C₁-C₈, C₁-C₆, C₁-C₄, or C₁-C₂). In embodiments, R² is unsubstituted unbranched saturated C₁-C₂₅ alkyl. In embodiments, R² is unsubstituted unbranched saturated C₁-C₂₀ alkyl. In embodiments, R² is unsubstituted unbranched saturated C₁-C₁₂ alkyl. In embodiments, R² is unsubstituted unbranched saturated C₁-C₈ alkyl. In embodiments, R² is unsubstituted unbranched saturated C₁-C₆ alkyl. In embodiments, R² is unsubstituted unbranched saturated C₁-C₄alkyl. In embodiments, R² is unsubstituted unbranched saturated C₁-C₂ alkyl.

In embodiments, R² is unsubstituted unbranched unsaturated alkyl (e.g., C₁-C₂₅, C₁-C₂₀, C₁-C₁₂, C₁-C₈, C₁-C₆, C₁-C₄, or C₁-C₂). In embodiments, R² is unsubstituted unbranched unsaturated C₁-C₂₅ alkyl. In embodiments, R² is unsubstituted unbranched unsaturated C₁-C₂₀ alkyl. In embodiments, R² is unsubstituted unbranched unsaturated C₁-C₁₂ alkyl. In embodiments, R² is unsubstituted unbranched unsaturated C₁-C₈ alkyl. In embodiments, R² is unsubstituted unbranched unsaturated C₁-C₆ alkyl. In embodiments, R² is unsubstituted unbranched unsaturated C₁-C₄ alkyl. In embodiments, R² is unsubstituted unbranched unsaturated C₁-C₂ alkyl.

In embodiments, R² is unsubstituted C₉-C₁₉ alkyl. In embodiments, R² is unsubstituted branched C₉-C₁₉ alkyl. In embodiments, R² is unsubstituted unbranched C₉-C₁₉ alkyl. In embodiments, R² is unsubstituted branched saturated C₉-C₁₉ alkyl. In embodiments, R² is unsubstituted branched unsaturated C₉-C₁₉ alkyl. In embodiments, R² is unsubstituted unbranched saturated C₉-C₁₉ alkyl. In embodiments, R² is unsubstituted unbranched unsaturated C₉-C₁₉ alkyl.

In embodiments, R³ is

hydrogen, —NH₂, —OH, —SH, —C(O)H, —C(O)NH₂, —NHC(O)H, —NHC(O)OH, —NHC(O)NH₂, —C(O)OH, —OC(O)H, —N₃, substituted (e.g., substituted with a substituent group, a size-limited substituent group, or lower substituent group) or unsubstituted alkyl (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkyl (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkyl (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkyl (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted aryl (e.g., C₆-C₁₂, C₆-C₁₀, or phenyl), or substituted (e.g., substituted with a substituent group, a size-limited substituent group, or lower substituent group) or unsubstituted heteroaryl (e.g., 5 to 12 membered, 5 to 10 membered, 5 to 9 membered, or 5 to 6 membered). In embodiments, R³ is hydrogen, —NH₂, —OH, —SH, —C(O)H, —C(O)NH₂, —NHC(O)H, —NHC(O)OH, —NHC(O)NH₂, —C(O)OH, —OC(O)H, —N₃, substituted (e.g., substituted with a substituent group, a size-limited substituent group, or lower substituent group) alkyl (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkyl (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkyl (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkyl (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) aryl (e.g., C₆-C₁₂, C₆-C₁₀, or phenyl), or substituted (e.g., substituted with a substituent group, a size-limited substituent group, or lower substituent group) heteroaryl (e.g., 5 to 12 membered, 5 to 10 membered, 5 to 9 membered, or 5 to 6 membered). In embodiments, R³ is
hydrogen, —NH₂, —OH, —SH, —C(O)H, —C(O)NH₂, —NHC(O)H, —NHC(O)OH, —NHC(O)NH₂, —C(O) OH, —OC(O)H, —N₃, unsubstituted alkyl (e.g., C₁-C₂₀, C₁-C₁₂, C₁-C₈, C₁-C₆, C₁-C₄, or C₁-C₂), unsubstituted heteroalkyl (e.g., 2 to 20 membered, 2 to 12 membered, 2 to 8 membered, 2 to 6 membered, 4 to 6 membered, 2 to 3 membered, or 4 to 5 membered), unsubstituted cycloalkyl (e.g., C₃-C₁₀, C₃-C₈, C₃-C₆, C₄-C₆, or C₅-C₆), unsubstituted heterocycloalkyl (e.g., 3 to 10 membered, 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). In embodiments, when R³ is substituted, R³ is substituted with a substituent group. In embodiments, when R³ is substituted, R³ is substituted with a size-limited substituent group. In embodiments, when R³ is substituted, R³ is substituted with a lower substituent group (e.g., oxo).

In embodiments, R^{8C} is hydrogen, substituted (e.g., substituted with a substituent group, a size-limited substituent group, or lower substituent group) or unsubstituted alkyl (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkyl (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkyl (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkyl (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted aryl (e.g., C₆-C₁₂, C₆-C₁₀, or phenyl), or substituted (e.g., substituted with a substituent group, a size-limited substituent group, or lower substituent group) or unsubstituted heteroaryl (e.g., 5 to 12 membered, 5 to 10 membered, 5 to 9 membered, or 5 to 6 membered). In embodiments, R^{8C} is hydrogen, substituted (e.g., substituted with a substituent group, a size-limited substituent group, or lower substituent group) alkyl (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkyl (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkyl (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkyl (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) aryl (e.g., C₆-C₁₂, C₆-C₁₀, or phenyl), or substituted (e.g., substituted with a substituent group, a size-limited substituent group, or lower substituent group) heteroaryl (e.g., 5 to 12 membered, 5 to 10 membered, 5 to 9 membered, or 5 to 6 membered). In embodiments, R^{8C} is hydrogen, unsubstituted alkyl (e.g., C₁-C₂₀, C₁-C₁₂, C₁-C₈, C₁-C₆, C₁-C₄, or C₁-C₂), unsubstituted heteroalkyl (e.g., 2 to 20 membered, 2 to 12 membered, 2 to 8 membered, 2 to 6 membered, 4 to 6 membered, 2 to 3 membered, or 4 to 5 membered), unsubstituted cycloalkyl (e.g., C₃-C₁₀, C₃-C₈, C₃-C₆, C₄-C₆, or C₅-C₆), unsubstituted heterocycloalkyl (e.g., 3 to 10 membered, 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). In embodiments, when R^{8C} is substituted, R^{8C} is substituted with a substituent group. In embodiments, when R^{8C} is substituted, R^{8C} is substituted with a size-limited substituent group. In embodiments, when R^{8C} is substituted, R^{8C} is substituted with a lower substituent group (e.g., oxo). In embodiments, R^{8C} is —COOH. In embodiments, R^{8C} is —CH₃.

In embodiments, R^{8D} is hydrogen, substituted (e.g., substituted with a substituent group, a size-limited substituent group, or lower substituent group) or unsubstituted alkyl (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkyl (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkyl (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkyl (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted aryl (e.g., C₆-C₁₂, C₆-C₁₀, or phenyl), or substituted (e.g., substituted with a substituent group, a size-limited substituent group, or lower substituent group) or unsubstituted heteroaryl (e.g., 5 to 12 membered, 5 to 10 membered, 5 to 9 membered, or 5 to 6 membered). In embodiments, R^{8D} is hydrogen, substituted (e.g., substituted with a substituent group, a size-limited substituent group, or lower substituent group) alkyl (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkyl (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkyl (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkyl (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) aryl (e.g., C₆-C₁₂, C₆-C₁₀, or phenyl), or substituted (e.g., substituted with a substituent group, a size-limited substituent group, or lower substituent group) heteroaryl (e.g., 5 to 12 membered, 5 to 10 membered, 5 to 9 membered, or 5 to 6 membered). In embodiments, R^{8D} is hydrogen, unsubstituted alkyl (e.g., C₁-C₂₀, C₁-C₁₂, C₁-C₈, C₁-C₆, C₁-C₄, or C₁-C₂), unsubstituted heteroalkyl (e.g., 2 to 20 membered, 2 to 12 membered, 2 to 8 membered, 2 to 6 membered, 4 to 6 membered, 2 to 3 membered, or 4 to 5 membered), unsubstituted cycloalkyl (e.g., C₃-C₁₀, C₃-C₈, C₃-C₆, C₄-C₆, or C₅-C₆), unsubstituted heterocycloalkyl (e.g., 3 to 10 membered, 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). In embodiments, when R^{8D} is substituted, R^{8D} is substituted with a substituent group. In embodiments, when R^{8D} is substituted, R^{8D} is substituted with a size-limited substituent group. In embodiments, when R^{8D} is substituted, R^{8D} is substituted with a lower substituent group (e.g., oxo). In embodiments, R^{8D} is —COOH.

In embodiments, R^{8E} is hydrogen, substituted (e.g., substituted with a substituent group, a size-limited substituent group, or lower substituent group) or unsubstituted alkyl (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkyl (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkyl (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkyl (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted aryl (e.g., C₆-C₁₂, C₆-C₁₀, or phenyl), or substituted (e.g., substituted with a substituent group, a size-limited substituent group, or lower substituent group) or unsubstituted heteroaryl (e.g., 5 to 12 membered, 5 to 10 membered, 5 to 9 membered, or 5 to 6 membered). In embodiments, R^{8E} is hydrogen, substituted (e.g., substituted with a substituent group, a size-limited substituent group, or lower substituent group) alkyl (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkyl (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkyl (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkyl (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) aryl (e.g., C₆-C₁₂, C₆-C₁₀, or phenyl), or substituted (e.g., substituted with a substituent group, a size-limited substituent group, or lower substituent group) heteroaryl (e.g., 5 to 12 membered, 5 to 10 membered, 5 to 9 membered, or 5 to 6 membered). In embodiments, R^{8E} is hydrogen, unsubstituted alkyl (e.g., C₁-C₂₀, C₁-C₁₂, C₁-C₈, C₁-C₆, C₁-C₄, or C₁-C₂), unsubstituted heteroalkyl (e.g., 2 to 20 membered, 2 to 12 membered, 2 to 8 membered, 2 to 6 membered, 4 to 6 membered, 2 to 3 membered, or 4 to 5 membered), unsubstituted cycloalkyl (e.g., C₃-C₁₀, C₃-C₈, C₃-C₆, C₄-C₆, or C₅-C₆), unsubstituted heterocycloalkyl (e.g., 3 to 10 membered, 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). In embodiments, when R^{8E} is substituted, R^{8E} is substituted with a substituent group. In embodiments, when R^{8E} is substituted, R^{8E} is substituted with a size-limited substituent group. In embodiments, when R^{8E} is substituted, R^{8E} is substituted with a lower substituent group (e.g., oxo). In embodiments, R^{8E} is —COOH.

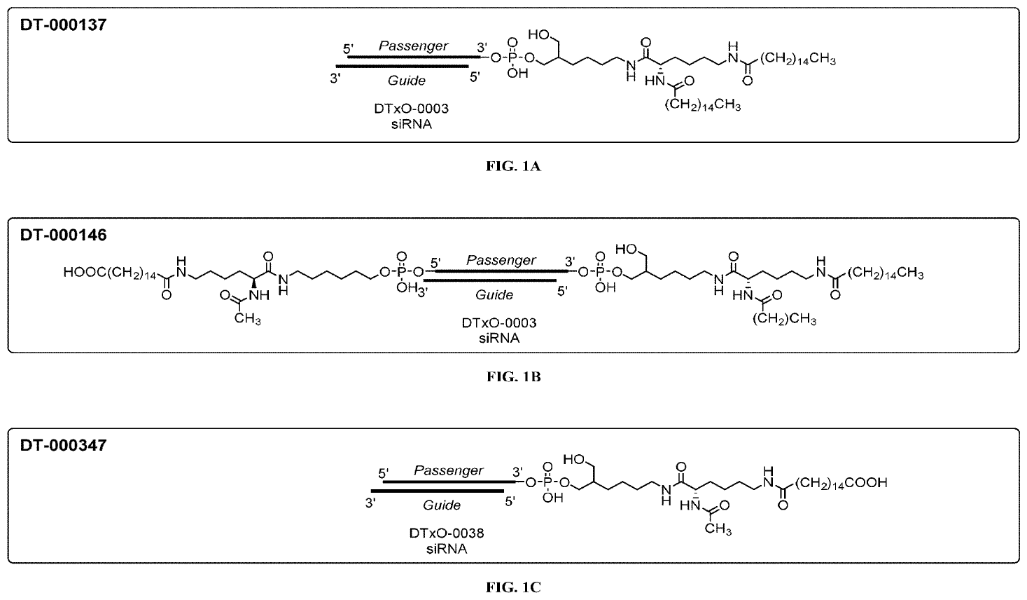

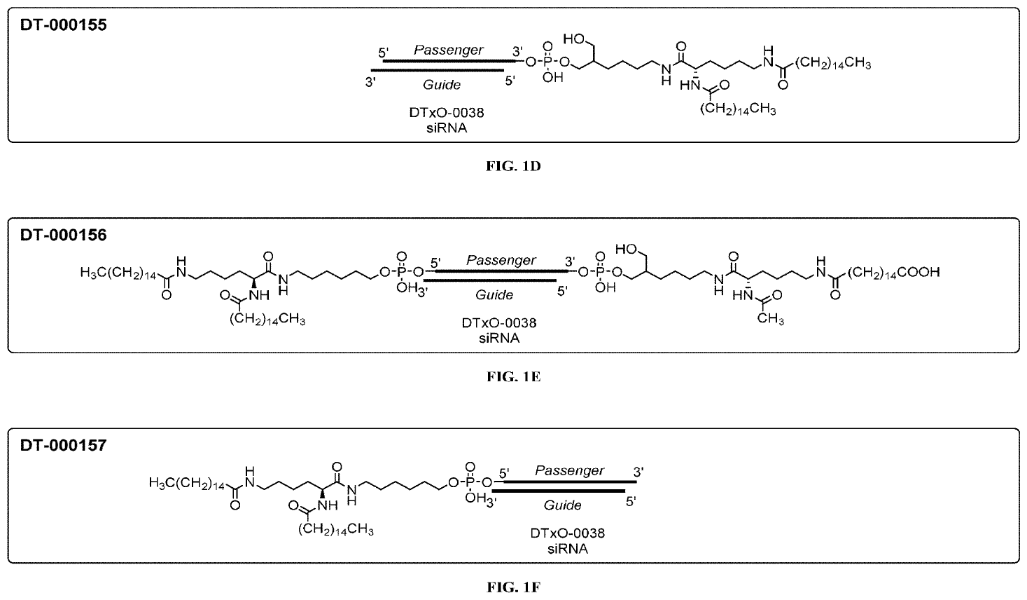

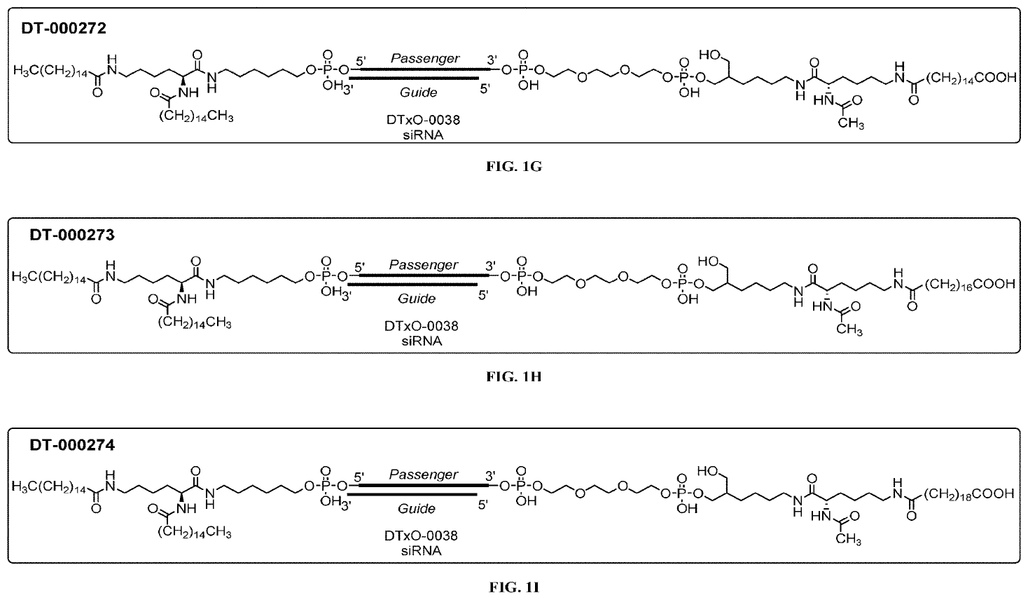

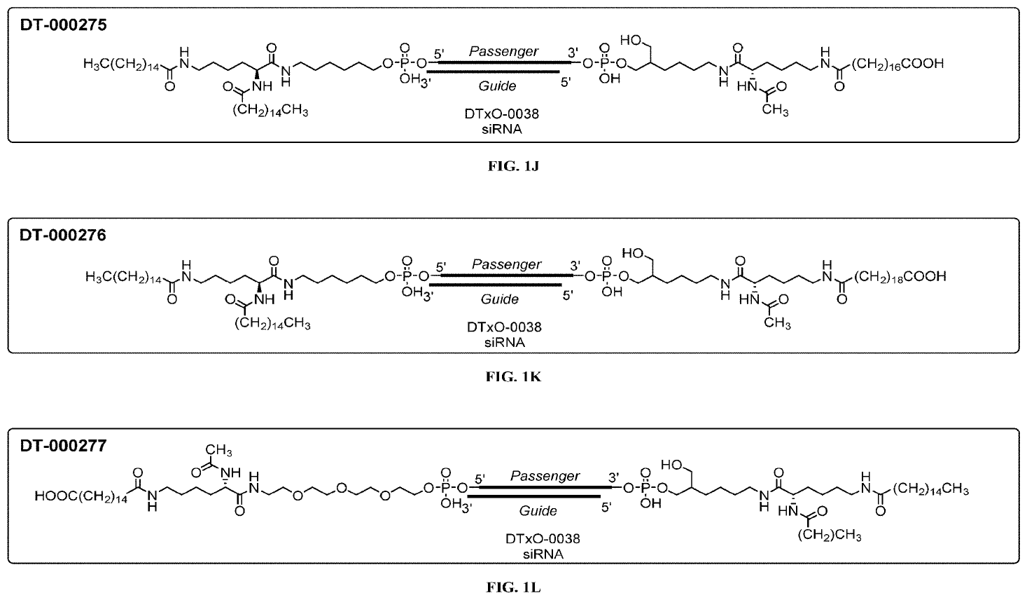

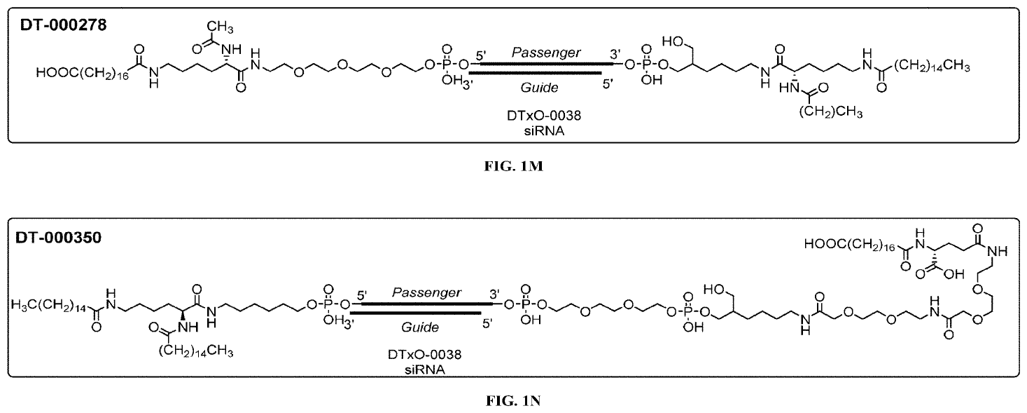

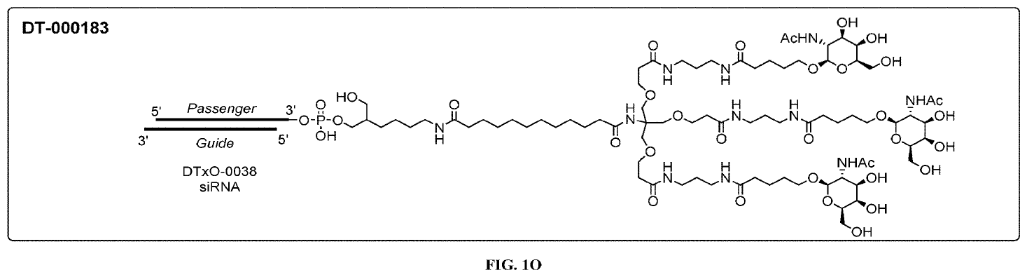

In embodiments, the compound including a nucleic acid includes a motif (e.g. formula (III), (III-a), (III-b), (III-c), (III-d), (III-e), (III-f), (III-g), (IV), or (IV-a)) described above including in any aspects, embodiments, claims, figures (e.g., FIGS. 1A-1O, tables (e.g., Tables 1-5 and A-H), examples, or schemes (e.g., Schemes I, II, III, and IV).

In embodiments, a half-life extension motif (HLEM) may be constructed with one or more independent -L²-COOH (e.g., structures shown in Table 1), which is joined to a scaffold (e.g., structures shows in Table 2) in L¹ or to a linker group L¹ (e.g., structures shown in Table 3). In embodiments, a scaffold and a linker may be combined or react to form L¹.

In embodiments, the compound including a nucleic acid includes one or more L²-COOH structures shown in Table 1 below.

TABLE 1
L² COOH Structures

| L² Description | L²-COOH Structure |
|---|---|
| C4 saturated | 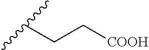 [SMILES: *CCC(=O)O] |
| C6 saturated | 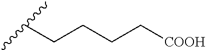 [SMILES: *CCCCC(=O)O] |
| C8 saturated | 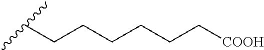 [SMILES: *CCCCCCC(=O)O] |
| C10 saturated | 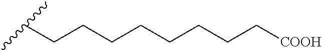 [SMILES: *CCCCCCCCC(=O)O] |
| C12 saturated | 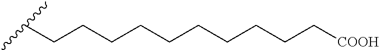 [SMILES: *CCCCCCCCCCC(=O)O] |
| C14 saturated | 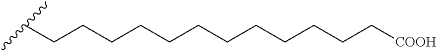 [SMILES: *CCCCCCCCCCCCC(=O)O] |
| C16 saturated | 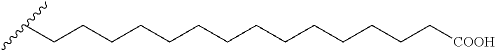 [SMILES: *CCCCCCCCCCCCCCC(=O)O] |
| C18 saturated | 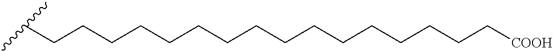 [SMILES: *CCCCCCCCCCCCCCCCC(=O)O] |
| C20 saturated | 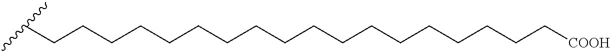 [SMILES: *CCCCCCCCCCCCCCCCCCC(=O)O] |
| C22 saturated | 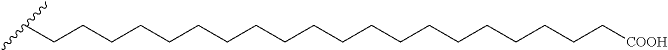 [SMILES: *CCCCCCCCCCCCCCCCCCCCC(=O)O] |
| C24 saturated | 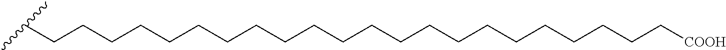 [SMILES: *CCCCCCCCCCCCCCCCCCCCCCC(=O)O] |
| C14 monounsat | 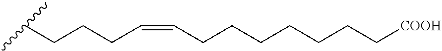 [SMILES: *CCC/C=C\CCCCCCCC(=O)O] |
| C14 polyunsat | 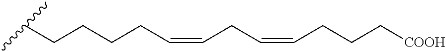 [SMILES: *CCCC/C=C\C/C=C\CCCC(=O)O] |
| C16 monounsat | 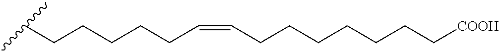 [SMILES: *CCCCC/C=C\CCCCCCCC(=O)O] |
| C16 tri-unsat | 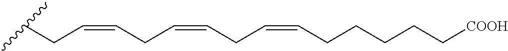 [SMILES: *C/C=C\C/C=C\C/C=C\CCCCCC(=O)O] |
| C18 oleate | 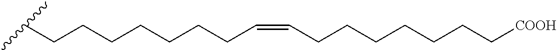 [SMILES: *CCCCCCC/C=C\CCCCCCCC(=O)O] |
| C18 lineolate | 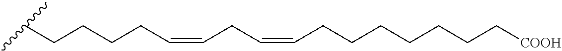 [SMILES: *CCCC/C=C\C/C=C\CCCCCCCC(=O)O] |
| C18 linolenic-α | 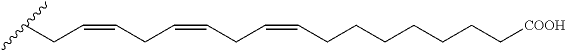 [SMILES: *C/C=C\C/C=C\C/C=C\CCCCCCCC(=O)O] |
| C18 linolenic-γ | 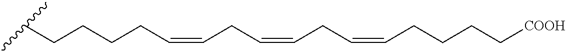 [SMILES: *CCCC/C=C\C/C=C\C/C=C\CCCCC(=O)O] |
| C20 eicosenoate | 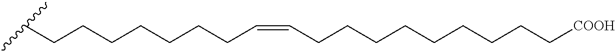 [SMILES: *CCCCCCC/C=C\CCCCCCCCCC(=O)O] |
| C20 eicosadienate | 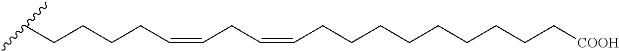 [SMILES: *CCCC/C=C\C/C=C\CCCCCCCCCC(=O)O] |
| C20 arachidonate | 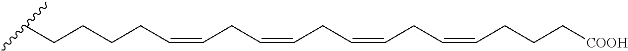 [SMILES: *CCCC/C=C\C/C=C\C/C=C\C/C=C\CCCC(=O)O] |
| C22 DHA | 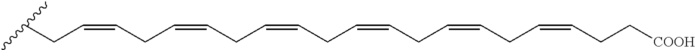 [SMILES: *C/C=C\C/C=C\C/C=C\C/C=C\C/C=C\C/C=C\CCC(=O)O] |

In embodiments, the compound including a nucleic acid includes a scaffold structure contributing to linking between L¹ and L² as shown in Table 2 below. In embodiments, the compound including a nucleic acid includes a scaffold DTx-01 (mono-v01) in Table 2. In embodiments, the compound including a nucleic acid includes a scaffold DTx-01 (mono-v02) in Table 2. In embodiments, the compound including a nucleic acid includes a scaffold DTx-01 (bis) in Table 2. In embodiments, the compound including a nucleic acid includes a scaffold DTx-03 (mono-v01) in Table 2. In embodiments, the compound including a nucleic acid includes a scaffold DTx-03 (mono-v02) in Table 2. In embodiments, the compound including a nucleic acid includes a scaffold DTx-03 (bis) in Table 2. In embodiments, the compound including a nucleic acid includes a scaffold DTx-06 (mono-v01) in Table 2. In embodiments, the compound including a nucleic acid includes a scaffold DTx-06 (mono-v02) in Table 2. In embodiments, the compound including a nucleic acid includes a scaffold DTx-06 (bis) in Table 2. In embodiments, the compound including a nucleic acid includes a scaffold DTx-08 (mono) in Table 2. In embodiments, the compound including a nucleic acid includes a scaffold DTx-08 (bis) in Table 2. In embodiments, the compound including a nucleic acid includes a scaffold DTx-09 (mono) in Table 2. In embodiments, the compound including a nucleic acid includes a scaffold DTx-09 (bis) in Table 2. In embodiments, the compound including a nucleic acid includes a scaffold DTx-10 (mono) in Table 2. In embodiments, the compound including a nucleic acid includes a scaffold DTx-10 (bis) in Table 2. In embodiments, the compound including a nucleic acid includes a scaffold DTx-11 (mono) in Table 2. In embodiments, the compound including a nucleic acid includes a scaffold DTx-11 (bis) in Table 2. In embodiments, the compound including a nucleic acid includes a scaffold DTx-12 in Table 2. In embodiments, the compound including a nucleic acid includes a scaffold DTx-13 in Table 2. In embodiments, the compound including a nucleic acid includes a scaffold DTx-14 in Table 2. In Table 2, squiggly lines represent attachment points to one or more fatty acid and to an atom constituting L¹.

TABLE 2
Scaffold Structures Contributing to L¹

| Scaffold Name | Scaffold Structure |
|---|---|
| DTx-01 (mono-v01) | 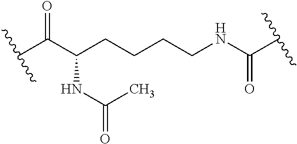 [SMILES: *C(=O)NCCCC[C@H](NC(C)=O)C(*)=O] |
| DTx-01 (mono-v02) | 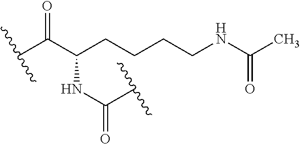 [SMILES: *C(=O)N[C@@H](CCCCNC(C)=O)C(*)=O] |
| DTx-01 (bis) | 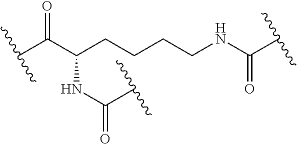 [SMILES: *C(=O)NCCCC[C@H](NC(*)=O)C(*)=O] |
| DTx-03 (mono-v01) | 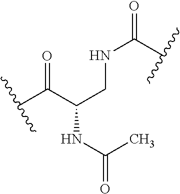 [SMILES: *C(=O)NC[C@H](NC(C)=O)C(*)=O] |
| DTx-03 (mono-v02) | 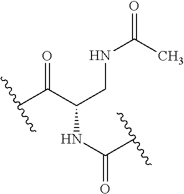 [SMILES: *C(=O)N[C@@H](CNC(C)=O)C(*)=O] |
| DTx-03 (bis) | 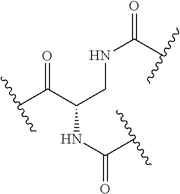 [SMILES: *C(=O)NC[C@H](NC(*)=O)C(*)=O] |
| DTx-06 (mono-v01) | 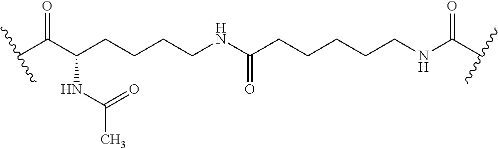 [SMILES: *C(=O)NCCCCCC(=O)NCCCC[C@H](NC(C)=O)C(*)=O] |
| DTx-06 (mono-v02) | 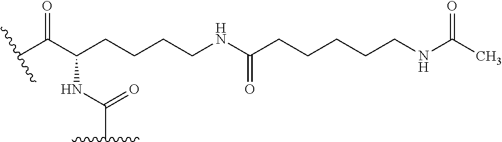 [SMILES: *C(=O)N[C@@H](CCCCNC(=O)CCCCCNC(C)=O)C(*)=O] |
| DTx-06 (bis) | 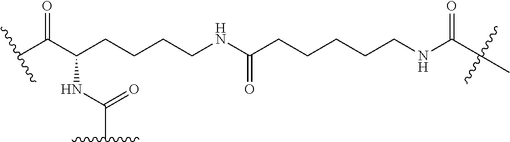 [SMILES: *C(=O)NCCCCCC(=O)NCCCC[C@H](NC(*)=O)C(*)=O] |
| DTx-08 (mono) | 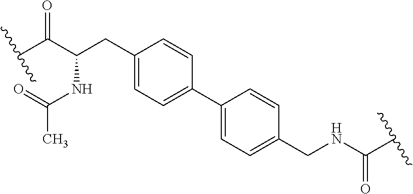 [SMILES: *C(=O)NCc1ccc(-c2ccc(C[C@H](NC(C)=O)C(*)=O)cc2)cc1] |
| DTx-08 (bis) | 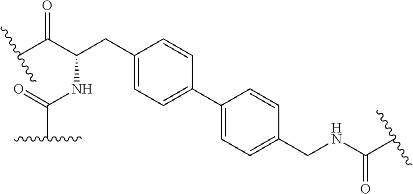 [SMILES: *C(=O)NCc1ccc(-c2ccc(C[C@H](NC(*)=O)C(*)=O)cc2)cc1] |
| DTx-09 (mono) | 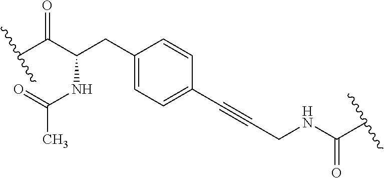 [SMILES: *C(=O)NCC#Cc1ccc(C[C@H](NC(C)=O)C(*)=O)cc1] |
| DTx-09 (bis) | 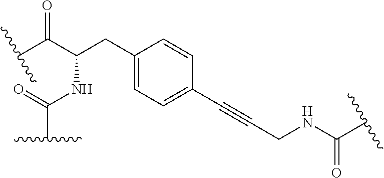 [SMILES: *C(=O)NCC#Cc1ccc(C[C@H](NC(*)=O)C(*)=O)cc1] |
| DTx-10 (mono) | 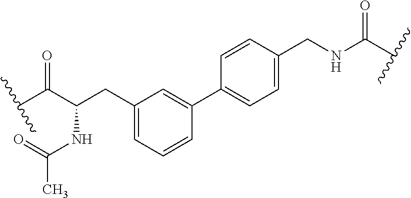 [SMILES: *C(=O)NCc1ccc(-c2cccc(C[C@H](NC(C)=O)C(*)=O)c2)cc1] |
| DTx-10 (bis) | 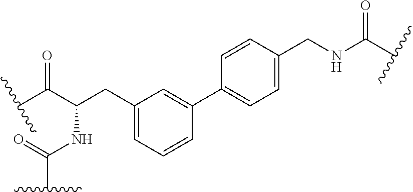 [SMILES: *C(=O)NCc1ccc(-c2cccc(C[C@H](NC(*)=O)C(*)=O)c2)cc1] |
| DTx-11 (mono) | 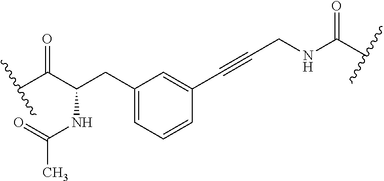 [SMILES: *C(=O)NCC#Cc1cccc(C[C@H](NC(C)=O)C(*)=O)c1] |
| DTx-11 (bis) | 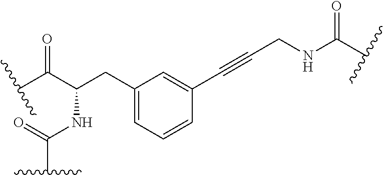 [SMILES: *C(=O)NCC#Cc1cccc(C[C@H](NC(*)=O)C(*)=O)c1] |
| DTx-12 | 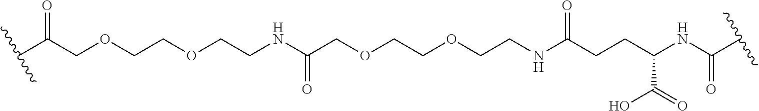 [SMILES: *C(=O)COCCOCCNC(=O)COCCOCCNC(=O)CC[C@H](NC(*)=O)C(=O)O] |
| DTx-13 | 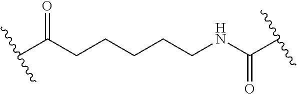 [SMILES: *C(=O)CCCCCNC(*)=O] |
| DTx-14 | 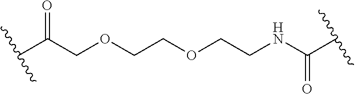 [SMILES: *C(=O)COCCOCCNC(*)=O] |

In embodiments, the compound including a nucleic acid includes a linker L¹ having a structure show in Table 3 below. In embodiments, the compound including a nucleic acid includes a linker C7 in Table 3. In embodiments, the compound including a nucleic acid includes a linker C6 in Table 3. In embodiments, the compound including a nucleic acid includes a linker C3 in Table 3. In embodiments, the compound including a nucleic acid includes a linker 6A-SER in Table 3. In embodiments, the compound including a nucleic acid includes a linker TEGN in Table 3. In embodiments, the compound including a nucleic acid includes a linker C12 in Table 3. In embodiments, the compound including a nucleic acid includes a linker AMC-N in Table 3. In embodiments, the compound including a nucleic acid includes a linker 5A-MEG in Table 3. In embodiments, the compound including a nucleic acid includes a linker AMC-P in Table 3. In embodiments, the compound including a nucleic acid includes a linker C6-P in Table 3. In embodiments, the compound including a nucleic acid includes a linker TEG-P in Table 3. In embodiments, the compound including a nucleic acid includes a linker PYR-C5-C3 in Table 3. In embodiments, the compound including a nucleic acid includes a linker PYR-DEG in Table 3. In embodiments, the compound including a nucleic acid includes a linker SP6-PO-C7 in Table 3. In embodiments, the compound including a nucleic acid includes a linker TEG9-PO-C7 in Table 3.

TABLE 3
Linker Structures Contributing to L¹

| Linker | |
|---|---|
| Abbreviation | Linker Structure |
| C7 | 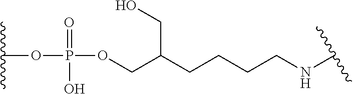 [SMILES: *NCCCCC(CO)COP(=O)(O)O*] |
| C6 | 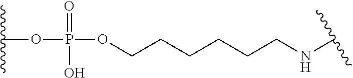 [SMILES: *NCCCCCCOP(=O)(O)O*] |
| C3 | 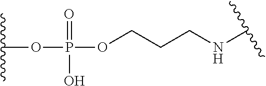 [SMILES: *NCCCOP(=O)(O)O*] |
| 6A-SER | 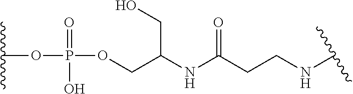 [SMILES: *NCCC(=O)NC(CO)COP(=O)(O)O*] |
| TEGN | 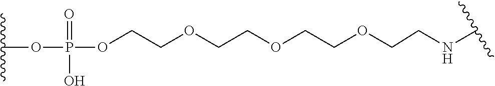 [SMILES: *NCCOCCOCCOCCOP(=O)(O)O*] |
| C12 | 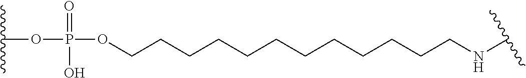 [SMILES: *NCCCCCCCCCCCCOP(=O)(O)O*] |
| AMC-N | 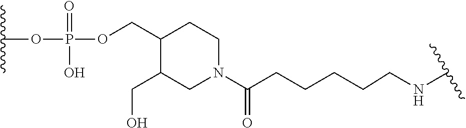 [SMILES: *NCCCCCC(=O)N1CCC(COP(=O)(O)O*)C(CO)C1] |
| 5A-MEG | 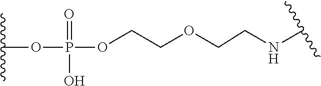 [SMILES: *NCCOCCOP(=O)(O)O*] |
| AMC-P | 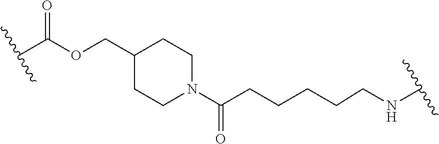 [SMILES: *NCCCCCC(=O)N1CCC(COC(*)=O)CC1] |
| C6-P | 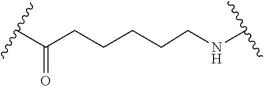 [SMILES: *NCCCCCC(*)=O] |
| TEG-P | 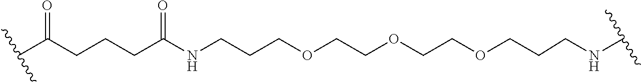 [SMILES: *NCCCOCCOCCOCCCNC(=O)CCCC(*)=O] |
| PTR-C5-C3 | 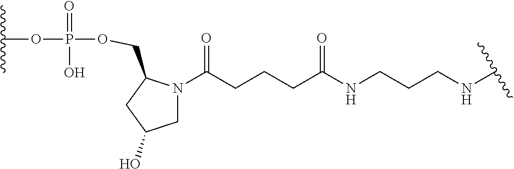 [SMILES: *NCCCNC(=O)CCCC(=O)N1C[C@H](O)C[C@H]1COP(=O)(O)O*] |
| PYR-DEG | 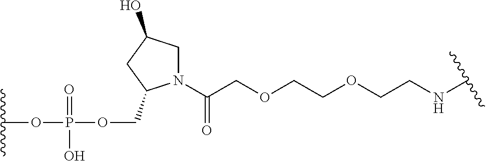 [SMILES: *NCCOCCOCC(=O)N1C[C@H](O)C[C@H]1COP(=O)(O)O*] |
| SP6-PO-C7 | 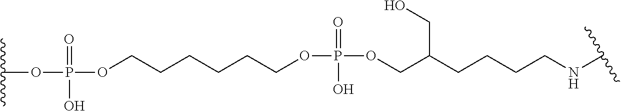 [SMILES: *NCCCCC(CO)COP(=O)(O)OCCCCCCOP(=O)(O)O*] |
| TEG9-PO-C7 | 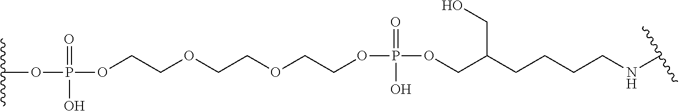 [SMILES: *NCCCCC(CO)COP(=O)(O)OCCOCCOCCOP(=O)(O)O*] |

In embodiments, L^{1A} is substituted or unsubstituted 2 to 8 membered heteroalkylene, L^{1B} is substituted or unsubstituted 5 to 6 membered heterocycloalkylene, and L^{1C} is substituted or unsubstituted heteroalkylene. In embodiments, L^{1A} is —OPO₂—O—CH₂—, —O—P(O)(S)—O—CH₂—, —O—P(O)(CH₃)—O—CH₂—, or —O—P(S)(CH₃)—CH₂—, L^{1B} is substituted or unsubstituted heterocycloalkylene, and L^{1C} is substituted or unsubstituted heteroalkylene. In embodiments, L^{1A} is —OPO₂—O—CH₂—, or —O—P(O)(S)—O—CH₂—. In embodiments, L^{1A} is —O—P(O)(CH₃)—O—CH₂—, or —O—P(S)(CH₃)—CH₂—. In embodiments, L^{1B} is substituted or unsubstituted 5 to 6 membered heterocycloalkylene. In embodiments, L^{1B} is substituted 5 to 6 membered heterocycloalkylene. In embodiments, L^{1B} is hydroxy (OH)-substituted 5 to 6 membered heterocycloalkylene. In embodiments, L^{1B} is unsubstituted 5 to 6 membered heterocycloalkylene. In embodiments, L^{1B} is substituted pyrrolidinylene. In embodiments, L^{1B} is hydroxy (OH)-substituted pyrrolidinylene. In embodiments, L^{1B} is unsubstituted pyrrolidinylene. In embodiments, L^{1B} is substituted piperidinylene. In embodiments, L^{1B} is hydroxy (OH)-substituted piperidinylene. In embodiments, L^{1B} is unsubstituted piperidinylene. In embodiments, L^{1C} is substituted or unsubstituted 2-12 membered heteroalkylene. In embodiments, L^{1C} is substituted 2-12 membered heteroalkylene. In embodiments, L^{1C} is oxo-substituted 2-12 membered heteroalkylene. In embodiments, L^{1C} is unsubstituted 2-12 membered heteroalkylene.

In embodiments, a uptake domain is represented by the structure of:

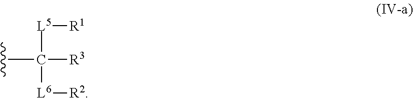

R¹, R², R³, L⁵, and L⁶ are as described above.

In embodiments, the compound including a nucleic acid includes one or more uptake domains having a structure shown in Table 4 below. In embodiments, the compound including a nucleic acid includes a DTx-01-01 domain in Table 4. In embodiments, the compound including a nucleic acid includes a DTx-01-03 domain 1 of Table 4. In embodiments, the compound including a nucleic acid includes a DTx-01-06 domain in Table 4. In embodiments, the compound including a nucleic acid includes a DTx-01-07 domain in Table 4. In embodiments, the compound including a nucleic acid includes a DTx-01-08 domain in Table 4. In embodiments, the compound including a nucleic acid includes a DTx-01-09 domain in Table 4. In embodiments, the compound including a nucleic acid includes a DTx-01-11 domain in Table 4. In embodiments, the compound including a nucleic acid includes a DTx-01-12 domain in Table 4. In embodiments, the compound including a nucleic acid includes a DTx-01-13 domain in Table 4. In embodiments, the compound including a nucleic acid includes a DTx-01-30 domain in Table 4. In embodiments, the compound including a nucleic acid includes a DTx-01-31 domain in Table 4. In embodiments, the compound including a nucleic acid includes a DTx-01-32 domain in Table 4. In embodiments, the compound including a nucleic acid includes a DTx-01-33 domain in Table 4. In embodiments, the compound including a nucleic acid includes a DTx-01-34 domain in Table 4. In embodiments, the compound including a nucleic acid includes a DTx-01-35 domain in Table 4. In embodiments, the compound including a nucleic acid includes a DTx-01-36 domain in Table 4. In embodiments, the compound including a nucleic acid includes a DTx-01-39 domain in Table 4. In embodiments, the compound including a nucleic acid includes a DTx-01-43 domain in Table 4. In embodiments, the compound including a nucleic acid includes a DTx-01-44 domain in Table 4. In embodiments, the compound including a nucleic acid includes a DTx-01-45 domain in Table 4. In embodiments, the compound including a nucleic acid includes a DTx-01-46 domain in Table 4. In embodiments, the compound including a nucleic acid includes a DTx-01-50 domain in Table 4. In embodiments, the compound including a nucleic acid includes a DTx-01-51 domain in Table 4. In embodiments, the compound including a nucleic acid includes a DTx-01-52 domain in Table 4. In embodiments, the compound including a nucleic acid includes a DTx-01-53 domain in Table 4. In embodiments, the compound including a nucleic acid includes a DTx-01-54 domain in Table 4. In embodiments, the compound including a nucleic acid includes a DTx-01-55 domain in Table 4. In embodiments, the compound including a nucleic acid includes a DTx-03-06 domain in Table 4. In embodiments, the compound including a nucleic acid includes a DTx-03-50 domain in Table 4. In embodiments, the compound including a nucleic acid includes a DTx-03-51 domain in Table 4. In embodiments, the compound including a nucleic acid includes a DTx-03-52 domain in Table 4. In embodiments, the compound including a nucleic acid includes a DTx-03-53 domain in Table 4. In embodiments, the compound including a nucleic acid includes a DTx-03-54 domain in Table 4. In embodiments, the compound including a nucleic acid includes a DTx-03-55 domain in Table 4. In embodiments, the compound including a nucleic acid includes a DTx-04-01 domain in Table 4. In embodiments, the compound including a nucleic acid includes a DTx-05-01 domain in Table 4. In embodiments, the compound including a nucleic acid includes a DTx-06-06 domain in Table 4. In embodiments, the compound including a nucleic acid includes a DTx-06-50 domain in Table 4. In embodiments, the compound including a nucleic acid includes a DTx-06-51 domain in Table 4. In embodiments, the compound including a nucleic acid includes a DTx-06-52 domain in Table 4. In embodiments, the compound including a nucleic acid includes a DTx-06-53 domain in Table 4. In embodiments, the compound including a nucleic acid includes a DTx-06-54 domain in Table 4. In embodiments, the compound including a nucleic acid includes a DTx-06-55 domain in Table 4. In embodiments, the compound including a nucleic acid includes a DTx-08-01 domain in Table 4. In embodiments, the compound including a nucleic acid includes a DTx-09-01 domain in Table 4. In embodiments, the compound including a nucleic acid includes a DTx-10-01 domain in Table 4. In embodiments, the compound including a nucleic acid includes a DTx-11-01 domain in Table 4. In embodiments, the compound including a nucleic acid includes a DTx-01-60 domain in Table 4. In embodiments, the compound including a nucleic acid includes a DTx-01-61 domain in Table 4. In embodiments, the compound including a nucleic acid includes a DTx-01-62 domain in Table 4. In embodiments, the compound including a nucleic acid includes a DTx-01-63 domain in Table 4. In embodiments, the compound including a nucleic acid includes a DTx-01-64 domain in Table 4. In embodiments, the compound including a nucleic acid includes a DTx-01-65 domain in Table 4. In embodiments, the compound including a nucleic acid includes a DTx-01-66 domain in Table 4. In embodiments, the compound including a nucleic acid includes a DTx-01-67 domain in Table 4. In embodiments, the compound including a nucleic acid includes a DTx-01-68 domain in Table 4. In embodiments, the compound including a nucleic acid includes a DTx-01-69 domain in Table 4. In embodiments, the compound including a nucleic acid includes a DTx-01-70 domain in Table 4. In embodiments, the compound including a nucleic acid includes a DTx-01-71 domain in Table 4. In embodiments, the compound including a nucleic acid includes a DTx-01-72 domain in Table 4. In embodiments, the compound including a nucleic acid includes a DTx-01-73 domain in Table 4. In embodiments, the compound including a nucleic acid includes a DTx-01-74 domain in Table 4. In embodiments, the compound including a nucleic acid includes a DTx-01-75 domain in Table 4. In embodiments, the compound including a nucleic acid includes a DTx-01-76 domain in Table 4. In embodiments, the compound including a nucleic acid includes a DTx-01-77 domain in Table 4. In embodiments, the compound including a nucleic acid includes a DTx-01-78 domain in Table 4. In embodiments, the compound including a nucleic acid includes a DTx-01-79 domain in Table 4. In embodiments, the compound including a nucleic acid includes a DTx-01-80 domain in Table 4. In embodiments, the compound including a nucleic acid includes a DTx-01-81 domain in Table 4. In embodiments, the compound including a nucleic acid includes a DTx-01-82 domain in Table 4. In embodiments, the compound including a nucleic acid includes a DTx-01-83 domain in Table 4. In embodiments, the compound including a nucleic acid includes a DTx-01-84 domain in Table 4. In embodiments, the compound including a nucleic acid includes a DTx-01-85 domain in Table 4. In embodiments, the compound including a nucleic acid includes a DTx-01-86 domain in Table 4. In embodiments, the compound including a nucleic acid includes a DTx-01-87 domain in Table 4. In embodiments, the compound including a nucleic acid includes a DTx-01-88 domain in Table 4. In embodiments, the compound including a nucleic acid includes a DTx-01-89 domain in Table 4. In embodiments, the compound including a nucleic acid includes a DTx-01-90 domain in Table 4. In embodiments, the compound including a nucleic acid includes a DTx-01-91 domain in Table 4. In embodiments, the compound including a nucleic acid includes a DTx-01-92 domain in Table 4. In embodiments, the compound including a nucleic acid includes a DTx-01-93 domain in Table 4. In embodiments, the compound including a nucleic acid includes a DTx-01-94 domain in Table 4. In embodiments, the compound including a nucleic acid includes a DTx-01-95 domain in Table 4. In embodiments, the compound including a nucleic acid includes a DTx-01-96 domain in Table 4. In embodiments, the compound including a nucleic acid includes a DTx-01-97 domain in Table 4. In embodiments, the compound including a nucleic acid includes a DTx-01-98 domain in Table 4. In embodiments, the compound including a nucleic acid includes a DTx-01-99 domain in Table 4. In embodiments, the compound including a nucleic acid includes a DTx-01-100 domain in Table 4. In embodiments, the compound including a nucleic acid includes a DTx-01-101 domain in Table 4.

TABLE 4
Uptake Domain

| Motif | |
|---|---|
| Name | Structure |
| DTx- 01-01 | 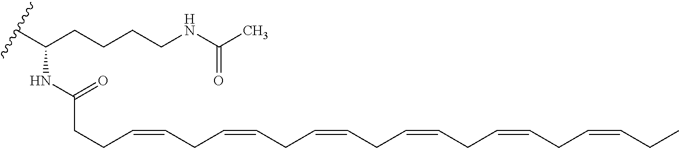 [SMILES: *[C@H](CCCCNC(C)=O)NC(=O)CC/C=C\C/C=C\C/C=C\C/C=C\C/C=C\C/C=C\CC] |
| DTx- 01-03 | 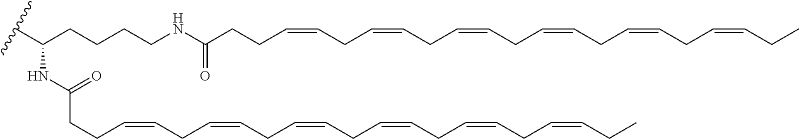 [SMILES: *[C@H](CCCCNC(=O)CC/C=C\C/C=C\C/C=C\C/C=C\C/C=C\C/C=C\CC)NC(=O)CC/C=C\C/C=C\C/C=C\C/C=C\C/C=C\C/C=C\CC] |
| DTx- 01-06 | 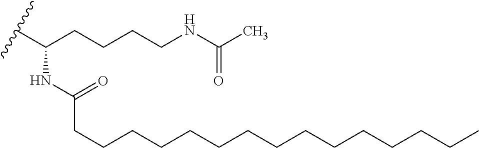 [SMILES: *[C@H](CCCCNC(C)=O)NC(=O)CCCCCCCCCCCCCCC] |
| DTx- 01-07 | 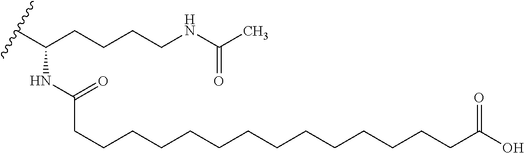 [SMILES: *[C@H](CCCCNC(C)=O)NC(=O)CCCCCCCCCCCCCCC(=O)O] |
| DTx- 01-08 | 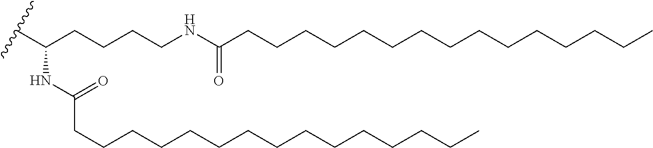 [SMILES: *[C@H](CCCCNC(=O)CCCCCCCCCCCCCCC)NC(=O)CCCCCCCCCCCCCCC] |
| DTx- 01-09 | 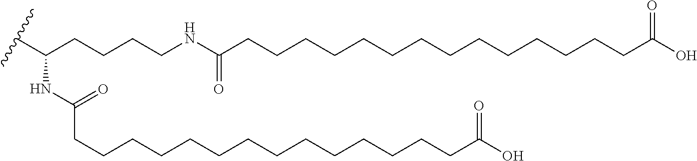 [SMILES: *[C@H](CCCCNC(=O)CCCCCCCCCCCCCCC(=O)O)NC(=O)CCCCCCCCCCCCCCC(=O)O] |
| DTx- 01-11 | 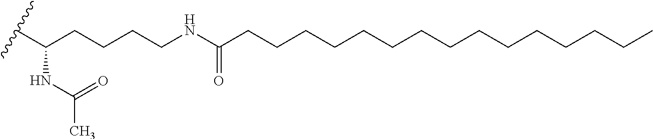 [SMILES: *[C@H](CCCCNC(=O)CCCCCCCCCCCCCCC)NC(C)=O] |
| DTx- 01-12 | 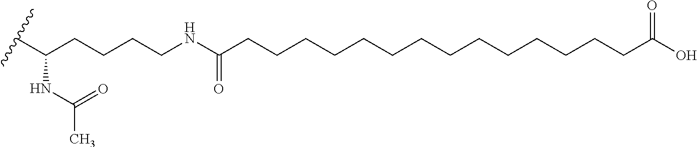 [SMILES: *[C@H](CCCCNC(=O)CCCCCCCCCCCCCCC(=O)O)NC(C)=O] |
| DTx- 01-13 | 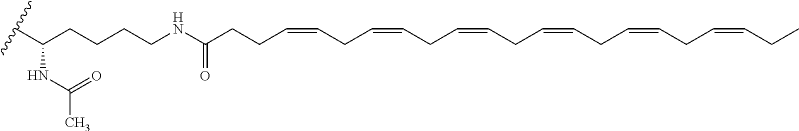 [SMILES: *[C@H](CCCCNC(=O)CC/C=C\C/C=C\C/C=C\C/C=C\C/C=C\C/C=C\CC)NC(C)=O] |
| DTx- 01-30 | 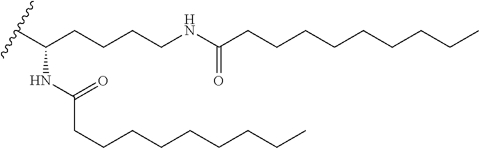 [SMILES: *[C@H](CCCCNC(=O)CCCCCCCCC)NC(=O)CCCCCCCCC] |
| DTx- 01-31 | 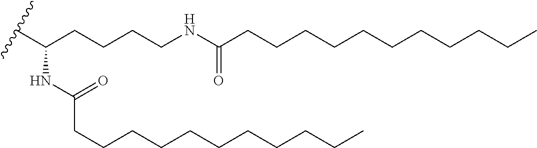 [SMILES: *[C@H](CCCCNC(=O)CCCCCCCCCCC)NC(=O)CCCCCCCCCCC] |
| DTx- 01-32 | 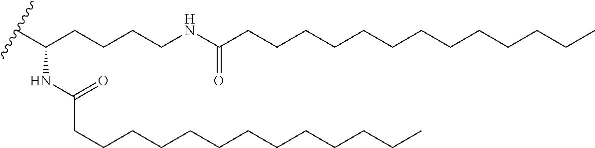 [SMILES: *[C@H](CCCCNC(=O)CCCCCCCCCCCCC)NC(=O)CCCCCCCCCCCCC] |
| DTx- 01-33 | 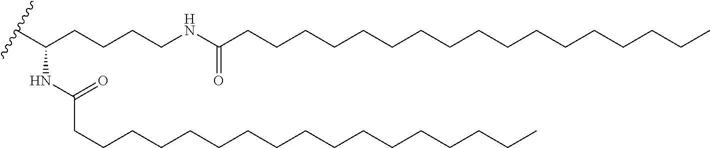 [SMILES: *[C@H](CCCCNC(=O)CCCCCCCCCCCCCCCCC)NC(=O)CCCCCCCCCCCCCCCCC] |
| DTx- 01-34 | 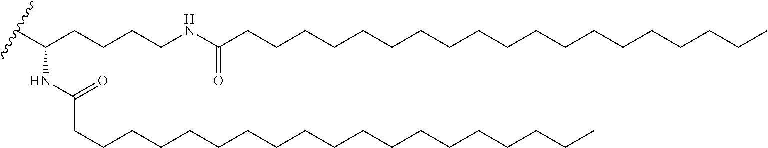 [SMILES: *[C@H](CCCCNC(=O)CCCCCCCCCCCCCCCCCCC)NC(=O)CCCCCCCCCCCCCCCCCCC] |
| DTx- 01-35 | 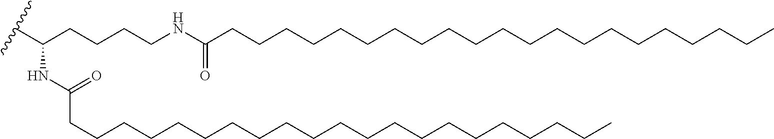 [SMILES: *[C@H](CCCCNC(=O)CCCCCCCCCCCCCCCCCCCCC)NC(=O)CCCCCCCCCCCCCCCCCCCCC] |
| DTx- 01-36 | 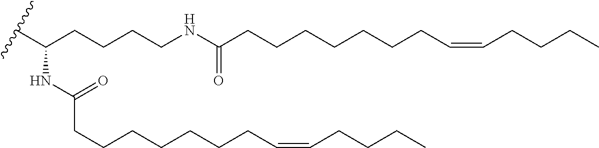 [SMILES: *[C@H](CCCCNC(=O)CCCCCCC/C=C\CCCC)NC(=O)CCCCCCC/C=C\CCCC] |
| DTx- 01-39 | 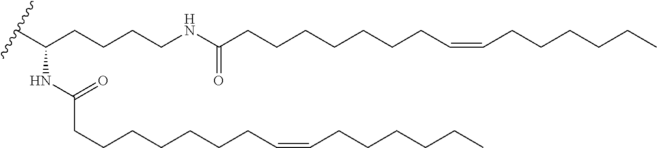 [SMILES: *[C@H](CCCCNC(=O)CCCCCCC/C=C\CCCCCC)NC(=O)CCCCCCC/C=C\CCCCCC] |
| DTx- 01-43 | 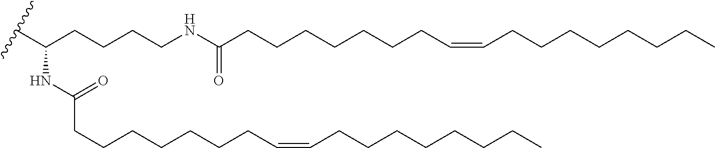 [SMILES: *[C@H](CCCCNC(=O)CCCCCCC/C=C\CCCCCCCC)NC(=O)CCCCCCC/C=C\CCCCCCCC] |
| DTx- 01-44 | 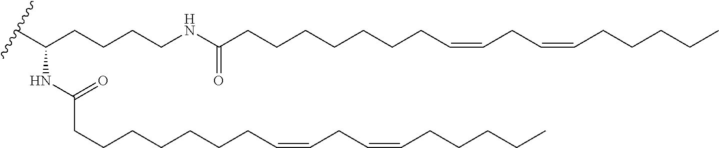 [SMILES: *[C@H](CCCCNC(=O)CCCCCCC/C=C\C/C=C\CCCCC)NC(=O)CCCCCCC/C=C\C/C=C\CCCCC] |
| DTx- 01-45 | 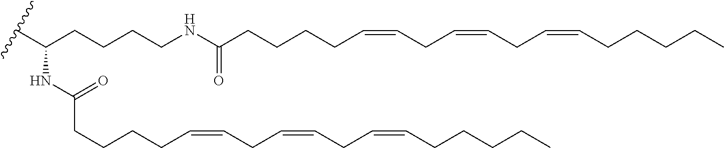 [SMILES: *[C@H](CCCCNC(=O)CCCC/C=C\C/C=C\C/C=C\CCCCC)NC(=O)CCCC/C=C\C/C=C\C/C=C\CCCCC] |
| DTx- 01-46 | 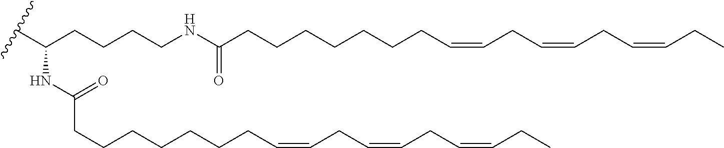 [SMILES: *[C@H](CCCCNC(=O)CCCCCCC/C=C\C/C=C\C/C=C\CC)NC(=O)CCCCCCC/C=C\C/C=C\C/C=C\CC] |
| DTx- 01-50 | 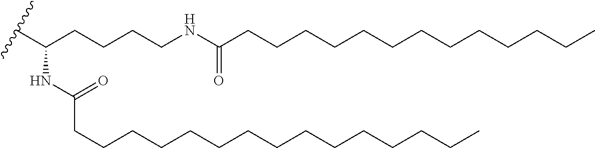 [SMILES: *[C@H](CCCCNC(=O)CCCCCCCCCCCCC)NC(=O)CCCCCCCCCCCCCCC] |
| DTx- 01-51 | 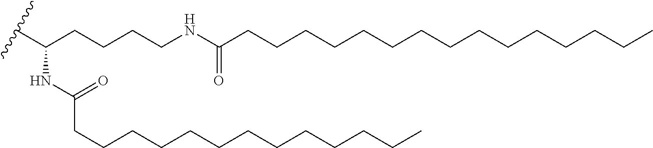 [SMILES: *[C@H](CCCCNC(=O)CCCCCCCCCCCCCCC)NC(=O)CCCCCCCCCCCCC] |
| DTx- 01-52 | 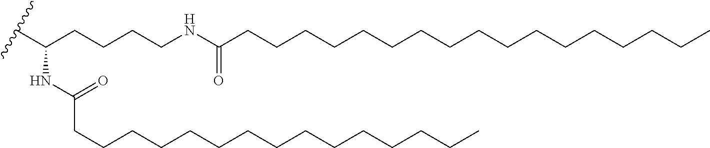 [SMILES: *[C@H](CCCCNC(=O)CCCCCCCCCCCCCCCCC)NC(=O)CCCCCCCCCCCCCCC] |
| DTx- 01-53 | 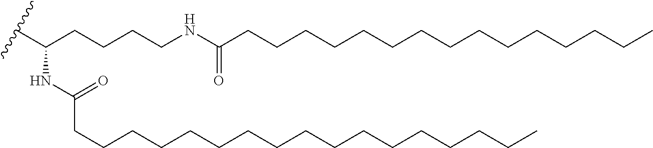 [SMILES: *[C@H](CCCCNC(=O)CCCCCCCCCCCCCCC)NC(=O)CCCCCCCCCCCCCCCCC] |
| DTx- 01-54 | 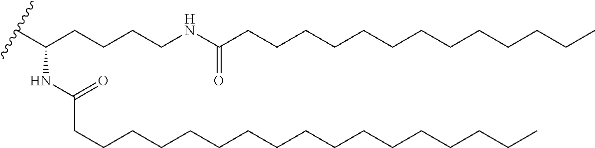 [SMILES: *[C@H](CCCCNC(=O)CCCCCCCCCCCCC)NC(=O)CCCCCCCCCCCCCCCCC] |
| DTx- 01-55 | 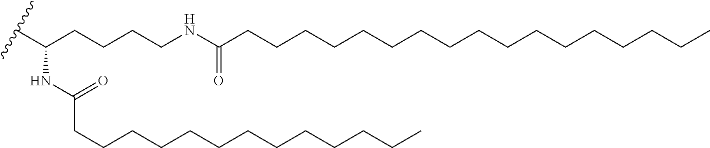 [SMILES: *[C@H](CCCCNC(=O)CCCCCCCCCCCCCCCCC)NC(=O)CCCCCCCCCCCCC] |
| DTx- 03-06 | 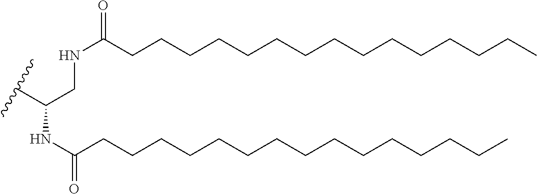 [SMILES: *[C@H](CNC(=O)CCCCCCCCCCCCCCC)NC(=O)CCCCCCCCCCCCCCC] |
| DTx- 03-50 | 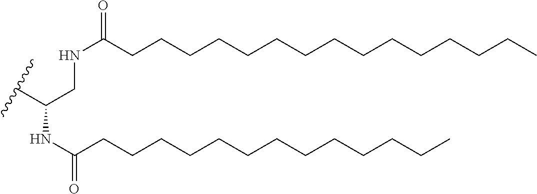 [SMILES: *[C@H](CNC(=O)CCCCCCCCCCCCCCC)NC(=O)CCCCCCCCCCCCC] |
| DTx- 03-51 | 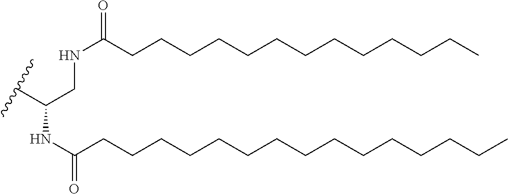 [SMILES: *[C@H](CNC(=O)CCCCCCCCCCCCC)NC(=O)CCCCCCCCCCCCCCC] |
| DTx- 03-52 | 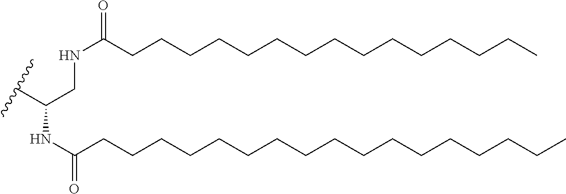 [SMILES: *[C@H](CNC(=O)CCCCCCCCCCCCCCC)NC(=O)CCCCCCCCCCCCCCCCC] |
| DTx- 03-53 | 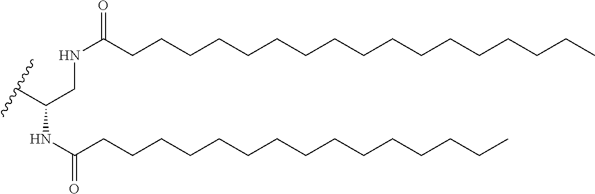 [SMILES: *[C@H](CNC(=O)CCCCCCCCCCCCCCCCC)NC(=O)CCCCCCCCCCCCCCC] |
| DTx- 03-54 | 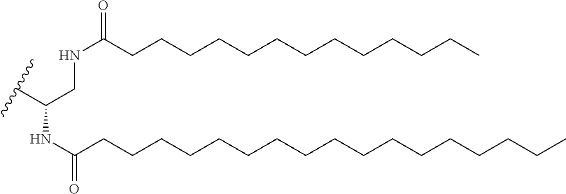 [SMILES: *[C@H](CNC(=O)CCCCCCCCCCCCC)NC(=O)CCCCCCCCCCCCCCCCC] |
| DTx- 03-55 | 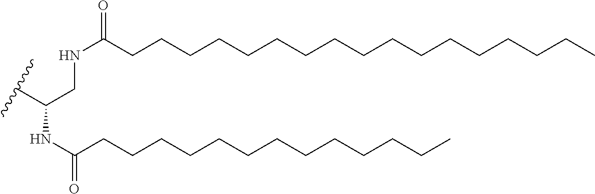 [SMILES: *[C@H](CNC(=O)CCCCCCCCCCCCCCCCC)NC(=O)CCCCCCCCCCCCC] |
| DTx- 04-01 | 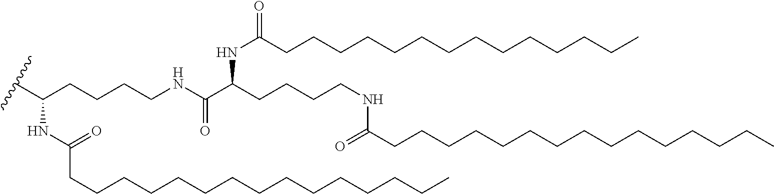 [SMILES: *[C@H](CCCCNC(=O)[C@H](CCCCNC(=O)CCCCCCCCCCCCCCC)NC(=O)CCCCCCCCCCCCCC)NC(=O)CCCCCCCCCCCCCCC] |
| DTx- 05-01 | 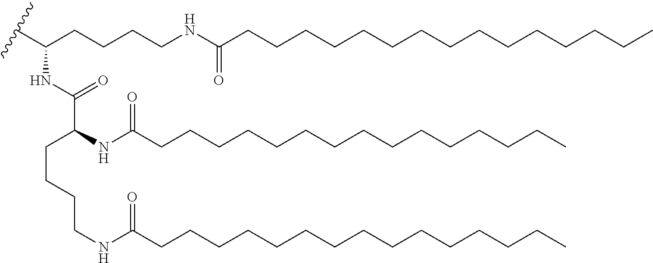 [SMILES: *[C@H](CCCCNC(=O)CCCCCCCCCCCCCCC)NC(=O)[C@H](CCCCNC(=O)CCCCCCCCCCCCCCC)NC(=O)CCCCCCCCCCCCCCC] |
| DTx- 06-06 | 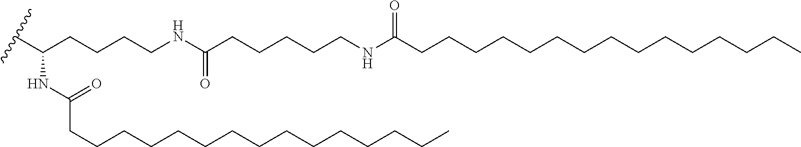 [SMILES: *[C@H](CCCCNC(=O)CCCCCNC(=O)CCCCCCCCCCCCCCC)NC(=O)CCCCCCCCCCCCCCC] |
| DTx- 06-50 | 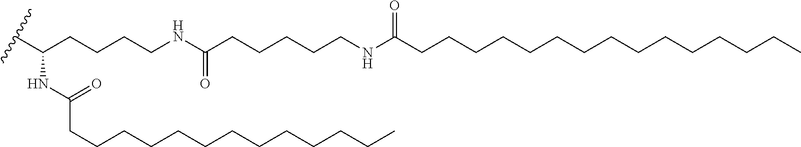 [SMILES: *[C@H](CCCCNC(=O)CCCCCNC(=O)CCCCCCCCCCCCCCC)NC(=O)CCCCCCCCCCCCC] |
| DTx- 06-51 | 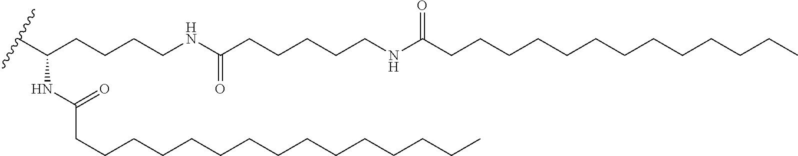 [SMILES: *[C@H](CCCCNC(=O)CCCCCNC(=O)CCCCCCCCCCCCC)NC(=O)CCCCCCCCCCCCCCC] |
| DTx- 06-52 | 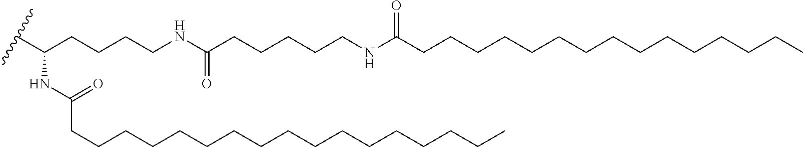 [SMILES: *[C@H](CCCCNC(=O)CCCCCNC(=O)CCCCCCCCCCCCCCC)NC(=O)CCCCCCCCCCCCCCCCC] |
| DTx- 06-53 | 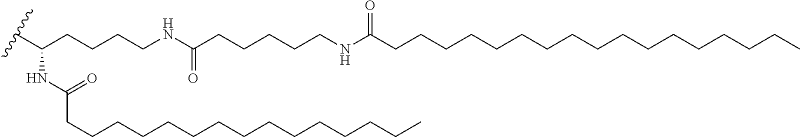 [SMILES: *[C@H](CCCCNC(=O)CCCCCNC(=O)CCCCCCCCCCCCCCCCC)NC(=O)CCCCCCCCCCCCCCC] |
| DTx- 06-54 | 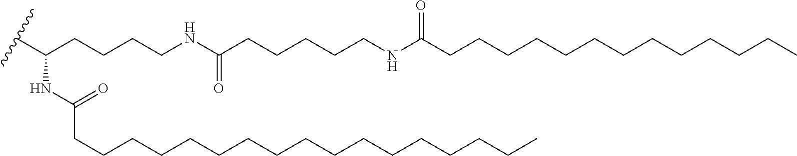 [SMILES: *[C@H](CCCCNC(=O)CCCCCNC(=O)CCCCCCCCCCCCC)NC(=O)CCCCCCCCCCCCCCCCC] |
| DTx- 06-55 | 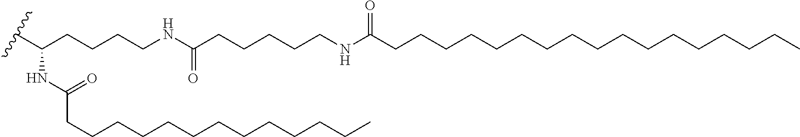 [SMILES: *[C@H](CCCCNC(=O)CCCCCNC(=O)CCCCCCCCCCCCCCCCC)NC(=O)CCCCCCCCCCCCC] |
| DTx- 08-01 | 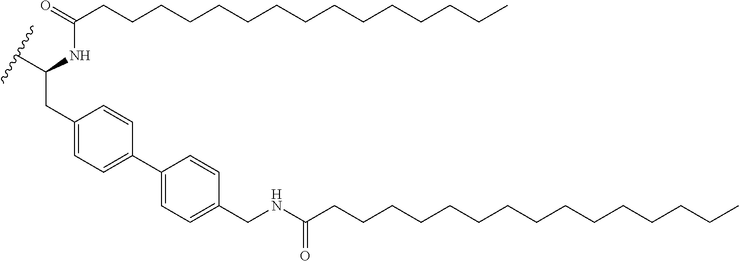 [SMILES: *[C@H](Cc1ccc(-c2ccc(CNC(=O)CCCCCCCCCCCCCCC)cc2)cc1)NC(=O)CCCCCCCCCCCCCCC] |
| DTx- 09-01 | 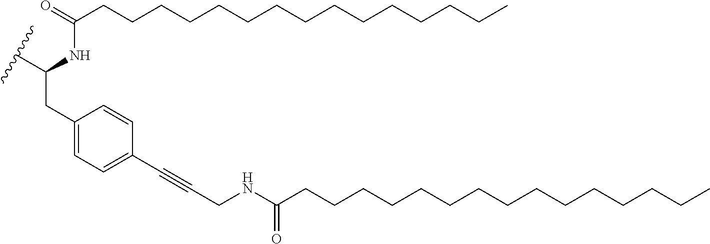 [SMILES: *[C@H](Cc1ccc(C#CCNC(=O)CCCCCCCCCCCCCCC)cc1)NC(=O)CCCCCCCCCCCCCCC] |
| DTx- 10-01 | 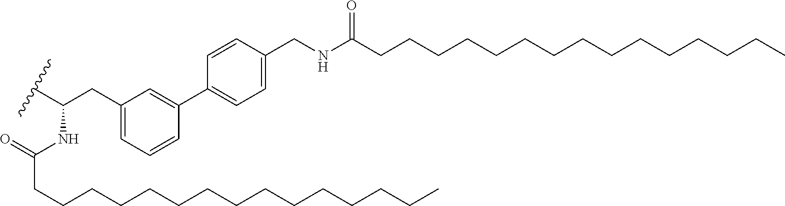 [SMILES: *[C@H](Cc1cccc(-c2ccc(CNC(=O)CCCCCCCCCCCCCCC)cc2)c1)NC(=O)CCCCCCCCCCCCCCC] |
| DTx- 11-01 | 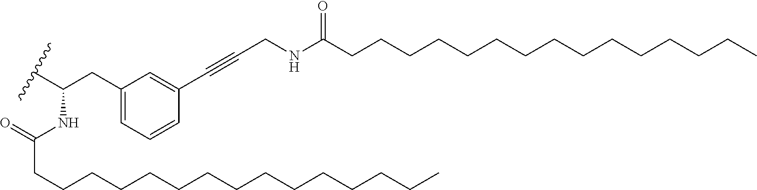 [SMILES: *[C@H](Cc1cccc(C#CCNC(=O)CCCCCCCCCCCCCCC)c1)NC(=O)CCCCCCCCCCCCCCC] |
| DTx- 01-60 | 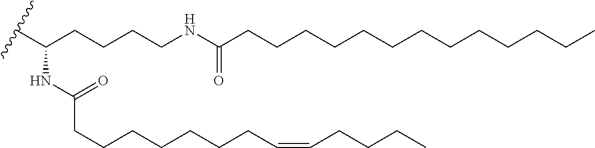 [SMILES: *[C@H](CCCCNC(=O)CCCCCCCCCCCCC)NC(=O)CCCCCCC/C=C\CCCC] |
| DTx- 01-61 | 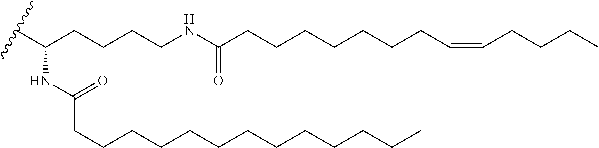 [SMILES: *[C@H](CCCCNC(=O)CCCCCCC/C=C\CCCC)NC(=O)CCCCCCCCCCCCC] |
| DTx- 01-62 | 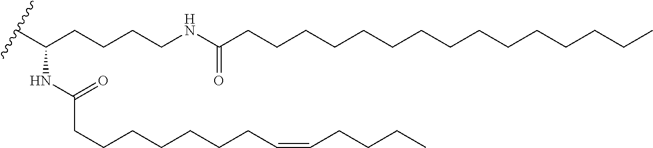 [SMILES: *[C@H](CCCCNC(=O)CCCCCCCCCCCCCCC)NC(=O)CCCCCCC/C=C\CCCC] |
| DTx- 01-63 | 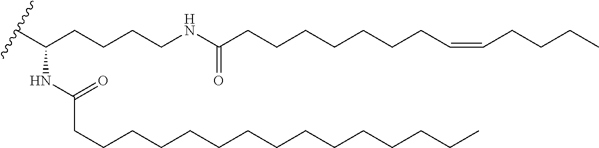 [SMILES: *[C@H](CCCCNC(=O)CCCCCCC/C=C\CCCC)NC(=O)CCCCCCCCCCCCCCC] |
| DTx- 01-64 | 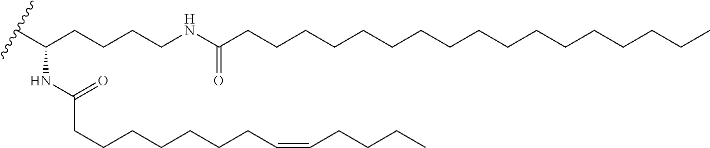 [SMILES: *[C@H](CCCCNC(=O)CCCCCCCCCCCCCCCCC)NC(=O)CCCCCCC/C=C\CCCC] |
| DTx- 01-65 | 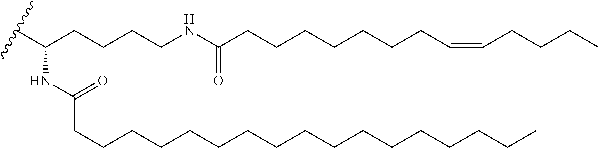 [SMILES: *[C@H](CCCCNC(=O)CCCCCCC/C=C\CCCC)NC(=O)CCCCCCCCCCCCCCCCC] |
| DTx- 01-66 | 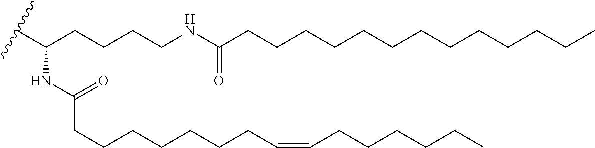 [SMILES: *[C@H](CCCCNC(=O)CCCCCCCCCCCCC)NC(=O)CCCCCCC/C=C\CCCCCC] |
| DTx- 01-67 | 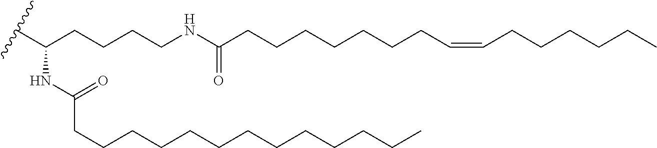 [SMILES: *[C@H](CCCCNC(=O)CCCCCCC/C=C\CCCCCC)NC(=O)CCCCCCCCCCCCC] |
| DTx- 01-68 | 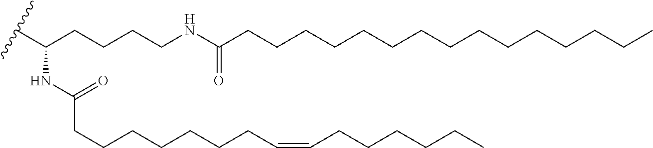 [SMILES: *[C@H](CCCCNC(=O)CCCCCCCCCCCCCCC)NC(=O)CCCCCCC/C=C\CCCCCC] |
| DTx- 01-69 | 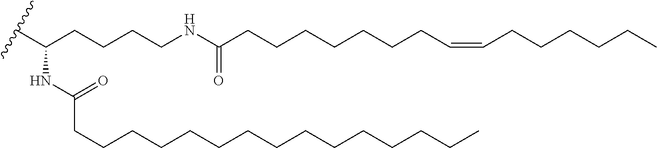 [SMILES: *[C@H](CCCCNC(=O)CCCCCCC/C=C\CCCCCC)NC(=O)CCCCCCCCCCCCCCC] |
| DTx- 01-70 | 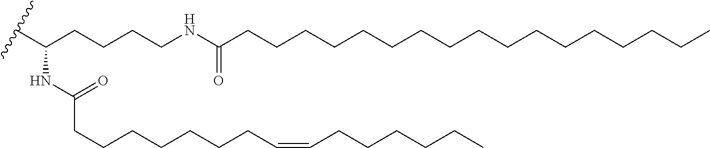 [SMILES: *[C@H](CCCCNC(=O)CCCCCCCCCCCCCCCCC)NC(=O)CCCCCCC/C=C\CCCCCC] |
| DTx- 01-71 | 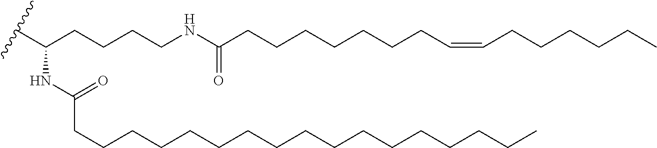 [SMILES: *[C@H](CCCCNC(=O)CCCCCCC/C=C\CCCCCC)NC(=O)CCCCCCCCCCCCCCCCC] |
| DTx- 01-72 | 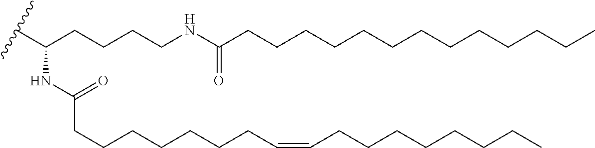 [SMILES: *[C@H](CCCCNC(=O)CCCCCCCCCCCCC)NC(=O)CCCCCCC/C=C\CCCCCCCC] |
| DTx- 01-73 | 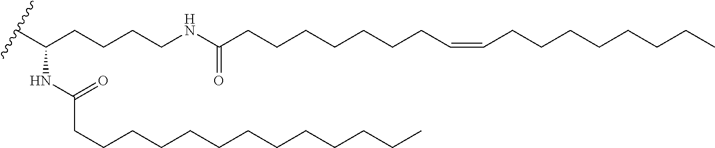 [SMILES: *[C@H](CCCCNC(=O)CCCCCCC/C=C\CCCCCCCC)NC(=O)CCCCCCCCCCCCC] |
| DTx- 01-74 | 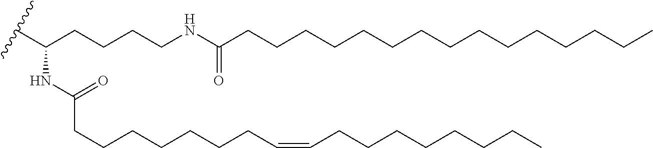 [SMILES: *[C@H](CCCCNC(=O)CCCCCCCCCCCCCCC)NC(=O)CCCCCCC/C=C\CCCCCCCC] |
| DTx- 01-75 | 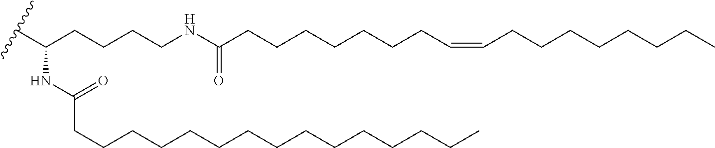 [SMILES: *[C@H](CCCCNC(=O)CCCCCCC/C=C\CCCCCCCC)NC(=O)CCCCCCCCCCCCCCC] |
| DTx- 01-76 | 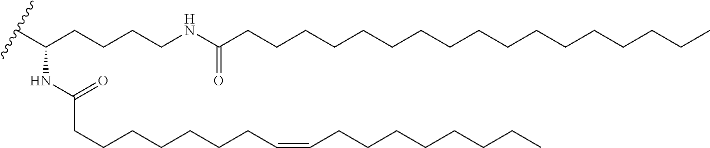 [SMILES: *[C@H](CCCCNC(=O)CCCCCCCCCCCCCCCCC)NC(=O)CCCCCCC/C=C\CCCCCCCC] |
| DTx- 01-77 | 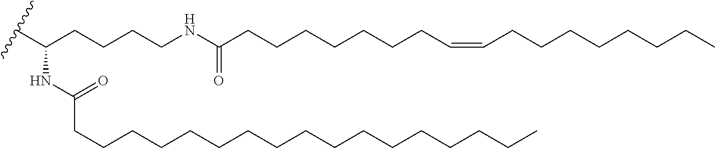 [SMILES: *[C@H](CCCCNC(=O)CCCCCCC/C=C\CCCCCCCC)NC(=O)CCCCCCCCCCCCCCCCC] |
| DTx- 01-78 | 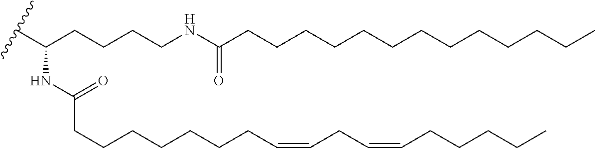 [SMILES: *[C@H](CCCCNC(=O)CCCCCCCCCCCCC)NC(=O)CCCCCCC/C=C\C/C=C\CCCCC] |
| DTx- 01-79 | 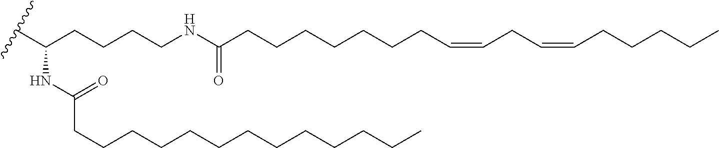 [SMILES: *[C@H](CCCCNC(=O)CCCCCCC/C=C\C/C=C\CCCCC)NC(=O)CCCCCCCCCCCCC] |
| DTx- 01-80 | 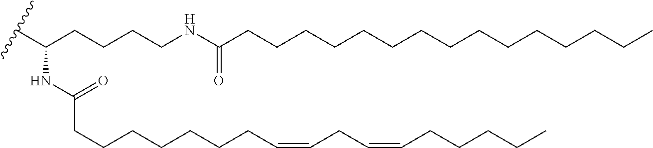 [SMILES: *[C@H](CCCCNC(=O)CCCCCCCCCCCCCCC)NC(=O)CCCCCCC/C=C\C/C=C\CCCCC] |
| DTx- 01-81 | 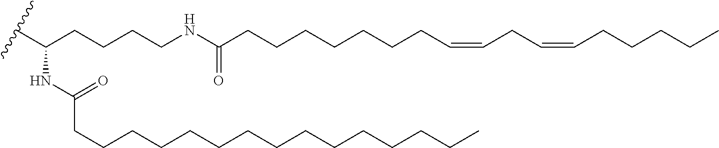 [SMILES: *[C@H](CCCCNC(=O)CCCCCCC/C=C\C/C=C\CCCCC)NC(=O)CCCCCCCCCCCCCCC] |
| DTx- 01-82 | 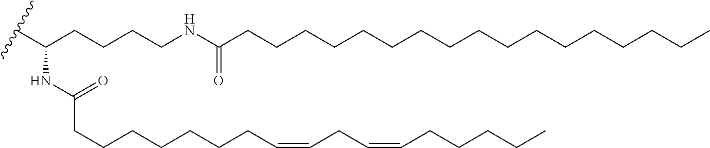 [SMILES: *[C@H](CCCCNC(=O)CCCCCCCCCCCCCCCCC)NC(=O)CCCCCCC/C=C\C/C=C\CCCCC] |
| DTx- 01-83 | 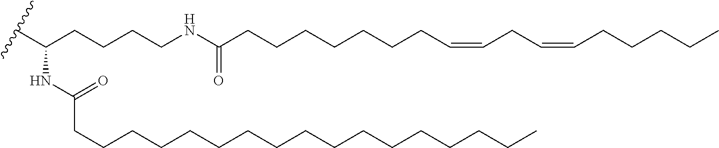 [SMILES: *[C@H](CCCCNC(=O)CCCCCCC/C=C\C/C=C\CCCCC)NC(=O)CCCCCCCCCCCCCCCCC] |
| DTx- 01-84 | 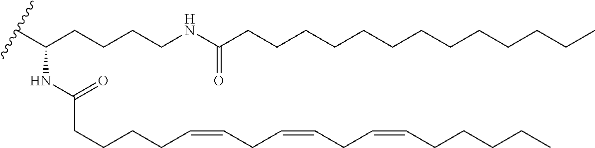 [SMILES: *[C@H](CCCCNC(=O)CCCCCCCCCCCCC)NC(=O)CCCC/C=C\C/C=C\C/C=C\CCCCC] |
| DTx- 01-85 | 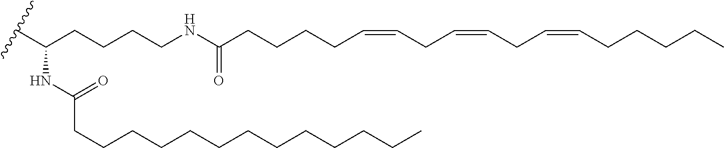 [SMILES: *[C@H](CCCCNC(=O)CCCC/C=C\C/C=C\C/C=C\CCCCC)NC(=O)CCCCCCCCCCCCC] |
| DTx- 01-86 | 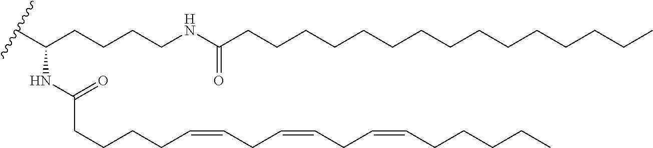 [SMILES: *[C@H](CCCCNC(=O)CCCCCCCCCCCCCCC)NC(=O)CCCC/C=C\C/C=C\C/C=C\CCCCC] |
| DTx- 01-87 | 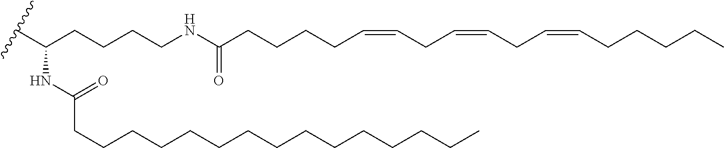 [SMILES: *[C@H](CCCCNC(=O)CCCC/C=C\C/C=C\C/C=C\CCCCC)NC(=O)CCCCCCCCCCCCCCC] |
| DTx- 01-88 | 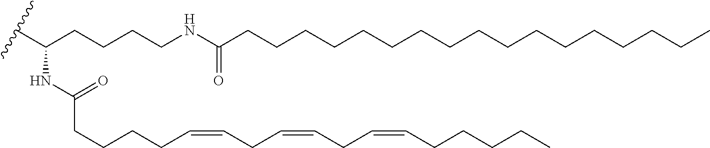 [SMILES: *[C@H](CCCCNC(=O)CCCCCCCCCCCCCCCCC)NC(=O)CCCC/C=C\C/C=C\C/C=C\CCCCC] |
| DTx- 01-89 | 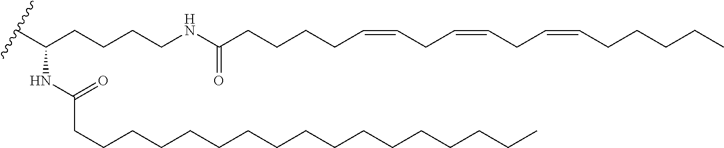 [SMILES: *[C@H](CCCCNC(=O)CCCC/C=C\C/C=C\C/C=C\CCCCC)NC(=O)CCCCCCCCCCCCCCCCC] |
| DTx- 01-90 | 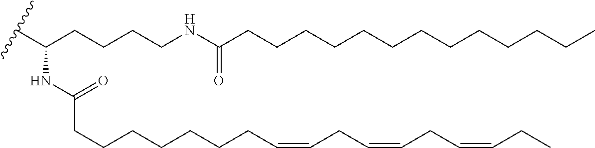 [SMILES: *[C@H](CCCCNC(=O)CCCCCCCCCCCCC)NC(=O)CCCCCCC/C=C\C/C=C\C/C=C\CC] |
| DTx- 01-91 | 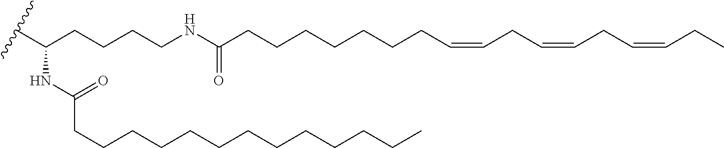 [SMILES: *[C@H](CCCCNC(=O)CCCCCCC/C=C\C/C=C\C/C=C\CC)NC(=O)CCCCCCCCCCCCC] |
| DTx- 01-92 | 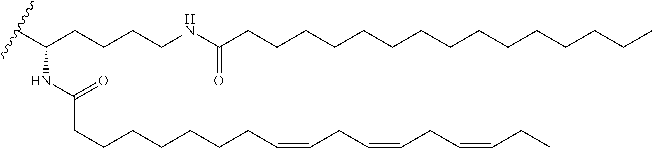 [SMILES: *[C@H](CCCCNC(=O)CCCCCCCCCCCCCCC)NC(=O)CCCCCCC/C=C\C/C=C\C/C=C\CC] |
| DTx- 01-93 | 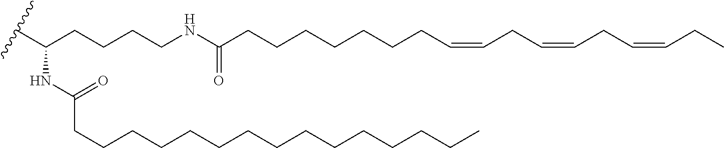 [SMILES: *[C@H](CCCCNC(=O)CCCCCCC/C=C\C/C=C\C/C=C\CC)NC(=O)CCCCCCCCCCCCCCC] |
| DTx- 01-94 | 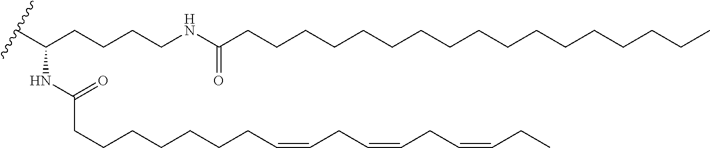 [SMILES: *[C@H](CCCCNC(=O)CCCCCCCCCCCCCCCCC)NC(=O)CCCCCCC/C=C\C/C=C\C/C=C\CC] |
| DTx- 01-95 | 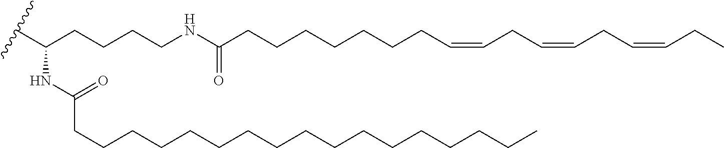 [SMILES: *[C@H](CCCCNC(=O)CCCCCCC/C=C\C/C=C\C/C=C\CC)NC(=O)CCCCCCCCCCCCCCCCC] |
| DTx- 01-96 | 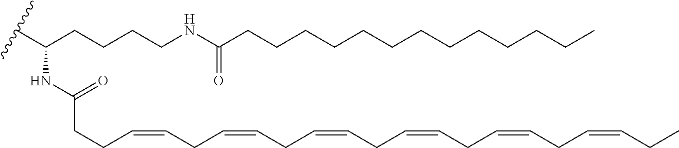 [SMILES: *[C@H](CCCCNC(=O)CCCCCCCCCCCCC)NC(=O)CC/C=C\C/C=C\C/C=C\C/C=C\C/C=C\C/C=C\CC] |
| DTx- 01-97 | 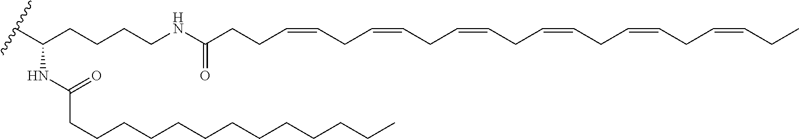 [SMILES: *[C@H](CCCCNC(=O)CC/C=C\C/C=C\C/C=C\C/C=C\C/C=C\C/C=C\CC)NC(=O)CCCCCCCCCCCCC] |
| DTx- 01-98 | 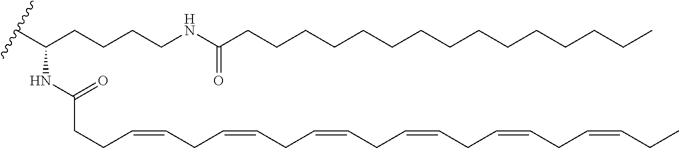 [SMILES: *[C@H](CCCCNC(=O)CCCCCCCCCCCCCCC)NC(=O)CC/C=C\C/C=C\C/C=C\C/C=C\C/C=C\C/C=C\CC] |
| DTx- 01-99 | 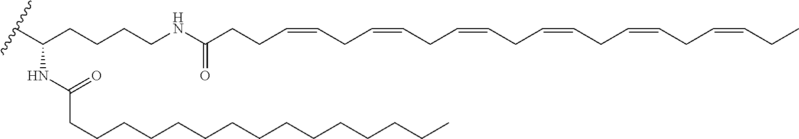 [SMILES: *[C@H](CCCCNC(=O)CC/C=C\C/C=C\C/C=C\C/C=C\C/C=C\C/C=C\CC)NC(=O)CCCCCCCCCCCCCCC] |
| DTx- 01-100 | 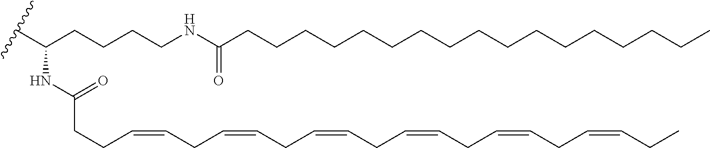 [SMILES: *[C@H](CCCCNC(=O)CCCCCCCCCCCCCCCCC)NC(=O)CC/C=C\C/C=C\C/C=C\C/C=C\C/C=C\C/C=C\CC] |
| DTx- 01-101v2 | 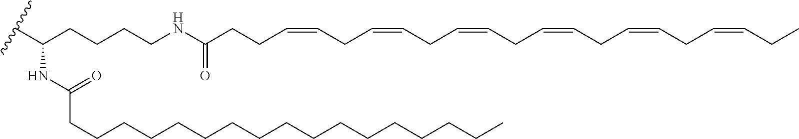 [SMILES: *[C@H](CCCCNC(=O)CC/C=C\C/C=C\C/C=C\C/C=C\C/C=C\C/C=C\CC)NC(=O)CCCCCCCCCCCCCCCCC] |

In embodiments, the oligonucleotide is targeted to a messenger RNA. In embodiments, the oligonucleotide is targeted to a pre-messenger RNA. In embodiments, the oligonucleotide is targeted to a long non-coding RNA.

In embodiments, the oligonucleotide is a single-stranded oligonucleotide. In embodiments, the oligonucleotide is a double-stranded oligonucleotide. In embodiments, the double-stranded oligonucleotide is a small interfering RNA (siRNA). In embodiments the double-stranded oligonucleotide is a short hairpin RNA (shRNA). In embodiments, the double-stranded oligonucleotide is a microRNA mimic.

In embodiments, the single-stranded oligonucleotide is an RNaseH oligonucleotide, which is dependent on RNaseH for cleavage of the mRNA to which it is complementary. In embodiments, the single-stranded oligonucleotide is a single-stranded small interfering RNA. In embodiments, the single-stranded oligonucleotide is an anti-microRNA oligonucleotide. In embodiments, the single-stranded oligonucleotide is a steric blocking oligonucleotide, which is an oligonucleotide that hybridizes to a target RNA and interferes with the target RNA activity, but does not cause degradation or cleavage of the target RNA. In embodiments, the single-stranded oligonucleotide is a CRISPR guide RNA. In embodiments, the single-stranded oligonucleotide is an aptamer.

In embodiments, the nucleic acid includes one or more modified nucleotides. In embodiments, the oligonucleotide includes one or more modified nucleotides. In embodiments, a modified nucleotide includes a modified sugar moiety. In embodiments, a modified nucleotide includes a modified internucleotide linkage. In embodiments, a modified nucleotide includes a modified nucleobase. In embodiments, a modified nucleotide includes a modified 5′-terminal phosphate group. In embodiments, a modified nucleotide includes a modification at the 5′ carbon of the pentafuranosyl sugar. In embodiments, a modified nucleotide includes a modification at the 3′ carbon of the pentafuranosyl sugar. In embodiments, a modified nucleotide includes a modification at the 2′ carbon of the pentafuranosyl sugar.

In embodiments, the nucleic acid includes one or more modified sugar moieties. In embodiments, the oligonucleotide includes one or more modified sugar moieties. In embodiments, a modified sugar moiety includes a 2′-modification, i.e. the sugar moiety is modified at the 2′ carbon of the pentafuranosyl sugar, relative to the naturally occurring 2′-OH of RNA or the 2′-H of DNA. In embodiments, a 2′-modification is selected from 2′-fluoro, 2′-OCF3, 2′-O—CH₃ (also referred to as “2′-OMe” or “2′-O-methyl”), 2′-OCH₂CH₂OCH₃ (also referred to as “2′-O-methoxyethyl” or “2′-MOE”), 2′-O(CH₂)₂SCH₃, O—(CH₂)₂—O—N(CH₃)₂, —O(CH₂)₂O(CH₂)₂N(CH₃)₂, and —O—CH₂—C(═O)—N(H)CH₃. In embodiments, the 2′ modification is a 2′-fluoro modification. In embodiments, the 2′ modification is a 2′-O-methyl modification. In embodiments, the 2′ modification is a 2′-O-methoxyethyl modification.

In embodiments, the 2′ modification is a bicyclic sugar modification, where the ribose has a covalent linkage between the 2′ and 4′ carbons. Nucleotides including such modified sugar moieties may be referred to as “bicyclic nucleic acids” or “BNA.” In embodiments, the covalent linkage of a bicyclic sugar modification is a 4′-CH₂—O-2′ linkage (methyleneoxy), also known as “LNA.” In embodiments, the covalent linkage of a bicyclic sugar modification is a 4′-(CH₂)₂—O-2′ linkage (ethyleneoxy), also known as “ENA.” In embodiments, the covalent linkage of a bicyclic sugar modification is a 4′-CH(CH₃)—O-2′ linkage (methyl(methyleneoxy)), also known as “constrained ethyl” or “cEt.” In embodiments, the covalent linkage of a bicyclic sugar modification is a 4′-CH(CH₂—OMe)-O-2′ linkage, also known as “c-MOE.” In embodiments, the covalent linkage of a bicyclic sugar modification is a 4′-CH₂—N(CH₃)—O-2′ linkage. In embodiments, the covalent linkage of a bicyclic sugar modification is a 4′-CH₂—N(H)—O-2′ linkage. In embodiments, the bicyclic sugar modification is a D sugar in the alpha configuration. In certain such embodiments, the bicyclic sugar modification is a D sugar in the beta configuration. In certain such embodiments, the bicyclic sugar modification is an L sugar in the alpha configuration. In certain such embodiments, the bicyclic sugar modification is an L sugar in the beta configuration.

In embodiments, a modified sugar moiety is an acyclic nucleoside derivative lacking the bond between the 2′ carbon and 3′ nitrogen of the sugar ring, also known as an “unlocked sugar modification.”

In embodiments, a modified sugar moiety is a morpholino moiety, where the pentafuranosyl sugar is replaced with a six-membered methylenemorpholine ring.

In embodiments, a modified nucleotide includes a modification at the 6′ carbon of the morpholino moiety. In embodiments, a modified nucleotide includes a modification at the 3′ nitrogen of the morpholino moiety. In embodiments, a modified nucleotide includes a modification at the 2′ carbon of the morpholino moiety.

In embodiments, the oxygen of the pentafuranosyl sugar is replace with a sulfur, to form a thio-sugar. In embodiments, a thio-sugar is modified at the 2′ carbon.

In embodiments, the nucleic acid includes one or more modified internucleotide linkages. In embodiments, the oligonucleotide includes one or more modified internucleotide linkages. In embodiments, a modified internucleotide linkage is a phosphorothioate linkage. In embodiments, a modified internucleotide linkage is a phosphorodiamidate linkage. In embodiments, a modified internucleotide linkage is a methylphosphonate internucleotide linkage. In embodiments, a modified internucleotide linkage is a boranophosphonate linkage. In embodiments, the modified internucleotide linkage is an O-methylphosphoroamidite linkage. In embodiments, the modified internucleotide linkage is a phosphoroamidate linkage. In embodiments, the nucleic acid contains a positive backbone. In embodiments, the nucleic acid contains a non-ionic backbone.

In embodiments, the nucleic acid includes one or more modified nucleobases. In embodiments, the oligonucleotide includes one or more modified nucleobase. In embodiments, a modified nucleobase is selected from 5-hydroxymethyl cytosine, 7-deazaguanine and 7-deazaadenine. In embodiments, a modified nucleobase is selected from 7-deaza-adenine, 7-deazaguanosine, 2-aminopyridine and 2-pyridone. In embodiments, a modified nucleobase is selected from 5-substituted pyrimidines, 6-azapyrimidines and N-2, N-6 and 0-6 substituted purines, including 2 aminopropyladenine, 5-propynyluracil and 5-propynylcytosine.

In embodiments, the 6′ carbon at the 5′ terminus of a nucleic acid includes a hydroxyl group, a phosphate group, or modified phosphate group. In embodiments, the 6′ carbon at the 5′ terminus of an oligonucleotide includes a hydroxyl group, a phosphate group, or modified phosphate group. In embodiments, the 5′-carbon at the 5′ terminus of an siRNA includes a hydroxyl group, a phosphate group, or modified phosphate group. In embodiments, the 5′ terminus of a single-stranded small interfering RNA includes a hydroxyl group, a phosphate group, or modified phosphate group. In embodiments, the modified phosphate group is 5′-(E)-vinylphosphonate.

In embodiments, the double-stranded oligonucleotide contains at least one 2′-O-methyl modification. In embodiments, the at least one 2′-O-methyl modification is present on the antisense strand, the sense strand, or both the antisense strand and the sense strand. In embodiments, the double-stranded oligonucleotide contains at least one 2′-fluoro modification. In embodiments, the at least one 2′-fluoro modification is present on the antisense strand, the sense strand, or both the antisense strand and the sense strand. In embodiments, the double-stranded oligonucleotide contains 2′-O-methyl modifications alternating with 2′-fluoro modifications. In embodiments, alternating sugar modifications are present on the antisense strand, the sense strand, or both the antisense strand and the sense strand. In embodiments, a double-stranded oligonucleotide contains three 2′-O-methyl modifications on the sense strand and three 2′-fluoro modifications on the antisense strand. In embodiments, every nucleotide in the double-stranded oligonucleotide includes either a 2′-O-methyl modification or a 2′-fluoro modification.

In embodiments, the double-stranded oligonucleotide contains at least one phosphorothioate linkage. In embodiments, the double-stranded oligonucleotide contains two to thirteen phosphorothioate linkages. In embodiments, the double-stranded oligonucleotide contains four phosphorothioate linkages. In embodiments, the double-stranded oligonucleotide contains two phosphorothioate linkages at the 3′ end of the antisense strand and two phosphorothioate linkages at the 3′end of the sense strand. In embodiments, a double-stranded oligonucleotide contains two phosphorothioate linkages at the 5′ end of the antisense strand and two phosphorothioate linkages at the 3′end of the sense strand. In embodiments, the double-stranded oligonucleotide contains five phosphorothioate linkages. In embodiments, the double-stranded oligonucleotide contains six phosphorothioate linkages. In embodiments, the double-stranded oligonucleotide contains seven phosphorothioate linkages. In embodiments, the double-stranded oligonucleotide contains eight phosphorothioate linkages. In embodiments, the double-stranded oligonucleotide contains nine phosphorothioate linkages. In embodiments, the double-stranded oligonucleotide contains ten phosphorothioate linkages. In embodiments, the double-stranded oligonucleotide contains eleven phosphorothioate linkages. In embodiments, the double-stranded oligonucleotide contains twelve phosphorothioate linkages. In embodiments, the double-stranded oligonucleotide contains thirteen phosphorothioate linkages. In embodiments, the double-stranded oligonucleotide contains two phosphorothioate linkages at the 3′ end of the antisense strand, seven phosphorothioate linkages at the 5′ end of the antisense strand, two phosphorothioate linkages at the 3′end of the sense strand, and two phosphorothioate linkages at the 5′end of the sense strand.

In embodiments, the double-stranded oligonucleotide may be conjugated at either of its 3′ ends to the HLEM-containing moiety portion of the compound. In embodiments, the double-stranded oligonucleotide is conjugated at the 3′end of its antisense strand to the HLEM-containing moiety portion. In embodiments, the double-stranded oligonucleotide is conjugated at the 3′end of its sense strand to the HLEM-containing moiety portion. In embodiments, the double-stranded oligonucleotide is conjugated to an HLEM-containing moiety at the 3′ end of its sense strand, and to an uptake motif at the 5′ end of its sense strand.

In embodiments, the double-stranded oligonucleotide is conjugated at either of its 5′ ends to the HLEM-containing moiety portion of the compound. In embodiments, the double-stranded oligonucleotide is conjugated at the 5′end of its antisense strand to the HLEM-containing moiety portion. In embodiments, the double-stranded oligonucleotide is conjugated at the 5′end of its sense strand to the HLEM-containing moiety portion. In embodiments, the double-stranded oligonucleotide is conjugated to an HLEM-containing moiety at the 5′ end of its sense strand, and to an uptake motif at the 3′ end of its sense strand.

In embodiments, the compound including a nucleic acid (A) is conjugated through a phosphodiester bond.

In embodiments, the double-stranded oligonucleotide is an siRNA including a 5′-(E)-vinylphosphonate group at the 5′ end of the antisense strand. In embodiments, the double-stranded oligonucleotide is a microRNA mimic including a 5′-(E)-vinylphosphonate group at the 5′ end of the antisense strand. In embodiments, the single-stranded oligonucleotide is a single-stranded small interfering RNA including a 5′-(E)-vinylphosphonate group at the 5′ end.

In embodiments, the oligonucleotide is a double-stranded oligonucleotide including an antisense strand hybridized to a sense strand and each of the antisense strand and sense strand is independently 15 to 30 nucleotides in length. In embodiments, each of the antisense strand and sense strand is 17 to 25 nucleotides in length. In embodiments, each of the antisense strand and sense strand is 19 to 23 nucleotides in length. In embodiments, the antisense strand is 15 nucleotides in length. In embodiments, the antisense strand is 16 nucleotides in length. In embodiments, the antisense strand is 17 nucleotides in length. In embodiments, the antisense strand is 18 nucleotides in length. In embodiments, the antisense strand is 19 nucleotides in length. In embodiments, the antisense strand is 20 nucleotides in length. In embodiments, the antisense strand is 21 nucleotides in length. In embodiments, the antisense strand is 22 nucleotides in length. In embodiments, the antisense strand is 23 nucleotides in length. In embodiments, the antisense strand is 24 nucleotides in length. In embodiments, the antisense strand is 25 nucleotides in length. In embodiments, the antisense strand is 26 nucleotides in length. In embodiments, the antisense strand is 27 nucleotides in length. In embodiments, the antisense strand is 28 nucleotides in length. In embodiments, the antisense strand is 29 nucleotides in length. In embodiments, the antisense strand is 30 nucleotides in length. In embodiments, the sense strand is 15 nucleotides in length. In embodiments, the antisense strand is 16 nucleotides in length. In embodiments, the sense strand is 17 nucleotides in length. In embodiments, the sense strand is 18 nucleotides in length. In embodiments, the sense strand is 19 nucleotides in length. In embodiments, the sense strand is 20 nucleotides in length. In embodiments, the sense strand is 21 nucleotides in length. In embodiments, the sense strand is 22 nucleotides in length. In embodiments, the sense strand is 23 nucleotides in length. In embodiments, the sense strand is 24 nucleotides in length. In embodiments, the sense strand is 25 nucleotides in length. In embodiments, the sense strand is 26 nucleotides in length. In embodiments, the sense strand is 27 nucleotides in length. In embodiments, the sense strand is 28 nucleotides in length. In embodiments, the sense strand is 29 nucleotides in length. In embodiments, the sense strand is 30 nucleotides in length.

In embodiments, the oligonucleotide is single-stranded and is 8 to 30 nucleotides in length. In embodiments, the single-stranded oligonucleotide is 12 to 25 nucleotides in length. In embodiments, the single-stranded oligonucleotide is 15 to 25 nucleotides in length. In embodiments, the single-stranded oligonucleotide is 17 to 23 nucleotides in length. In embodiments, the single-stranded oligonucleotide is 8 nucleotides in length. In embodiments, the single-stranded oligonucleotide is 8 nucleotides in length. In embodiments, the single-stranded oligonucleotide is 9 nucleotides in length. In embodiments, the single-stranded oligonucleotide is 10 nucleotides in length. In embodiments, the single-stranded oligonucleotide is 11 nucleotides in length. In embodiments, the single-stranded oligonucleotide is 12 nucleotides in length. In embodiments, the single-stranded oligonucleotide is 13 nucleotides in length. In embodiments, the single-stranded oligonucleotide is 14 nucleotides in length. In embodiments, the single-stranded oligonucleotide is 15 nucleotides in length. In embodiments, the single-stranded oligonucleotide is 16 nucleotides in length. In embodiments, the single-stranded oligonucleotide is 17 nucleotides in length. In embodiments, the single-stranded oligonucleotide is 18 nucleotides in length. In embodiments, the single-stranded oligonucleotide is 19 nucleotides in length. In embodiments, the single-stranded oligonucleotide is 20 nucleotides in length. In embodiments, the single-stranded oligonucleotide is 21 nucleotides in length. In embodiments, the single-stranded oligonucleotide is 22 nucleotides in length. In embodiments, the single-stranded oligonucleotide is 23 nucleotides in length. In embodiments, the single-stranded oligonucleotide is 24 nucleotides in length. In embodiments, the single-stranded oligonucleotide is 25 nucleotides in length. In embodiments, the single-stranded oligonucleotide is 26 nucleotides in length. In embodiments, the single-stranded oligonucleotide is 27 nucleotides in length. In embodiments, the single-stranded oligonucleotide is 28 nucleotides in length. In embodiments, the single-stranded oligonucleotide is 29 nucleotides in length. In embodiments, the single-stranded oligonucleotide is 30 nucleotides in length.

In embodiments, the compound is capable of binding a serum protein. In embodiments, the compound is capable of binding serum albumin. In embodiments, the compound has increased serum albumin binding compared to an identical compound lacking the one or more optionally different half-life extension motifs. In embodiments, the compound has an increased serum half-life compared to an identical compound lacking the one or more optionally different half-life extension motifs.

In embodiments, the compound further includes a ligand. In embodiments, the ligand may include one or more selected from a synthetic compound, a peptide, an antibody, a carbohydrate, or an additional nucleic acid. In embodiments, the ligand may include one or more selected from a peptide, an antibody, a carbohydrate, or an additional nucleic acid. In embodiments, the uptake motif independently includes one or more selected from a peptide, an antibody, a carbohydrate, or an additional nucleic acid. In embodiments, one or more uptake motifs include one or more selected from a peptide, an antibody, a carbohydrate, or an additional nucleic acid. In embodiments, the ligand may replace the uptake motif. In embodiments, one or more ligand may replace one or more uptake motifs.

### Pharmaceutical Compositions

Also provided herein are pharmaceutical formulations or pharmaceutical composition. In embodiments, the pharmaceutical formulation (e.g., composition) includes a compound (e.g. formula (I), (II), (III), (III-a), (III-b), (III-c), (III-d), (III-e), (III-f), (III-g), (IV), or (IV-a)) described above including in any aspects, embodiments, claims, figures (e.g., FIGS. 1A-1O, tables (e.g., Tables 1-5 and A-H), examples, or schemes (e.g., Schemes I, II, III, and IV), and a pharmaceutically acceptable excipient.

The pharmaceutical composition may be prepared and administered in a wide variety of dosage formulations. Compounds described may be administered orally, rectally, or by injection (e.g. intravenously, intramuscularly, intracutaneously, subcutaneously, intraduodenally, or intraperitoneally).

For preparing pharmaceutical compositions from compounds described herein, pharmaceutically acceptable carriers can be either solid or liquid. Solid form preparations include powders, tablets, pills, capsules, cachets, suppositories, and dispersible granules. A solid carrier may be one or more substance that may also act as diluents, flavoring agents, binders, preservatives, tablet disintegrating agents, or an encapsulating material.

In powders, the carrier may be a finely divided solid in a mixture with the finely divided active component. In tablets, the active component may be mixed with the carrier having the necessary binding properties in suitable proportions and compacted in the shape and size desired.

The powders and tablets preferably contain from 5% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations that are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted according to the particular application and the potency of the active component. The composition can, if desired, also contain other compatible therapeutic agents.

Some compounds may have limited solubility in water and therefore may require a surfactant or other appropriate co-solvent in the composition. Such co-solvents include: Polysorbate 20, 60, and 80; Pluronic F-68, F-84, and P-103; cyclodextrin; and polyoxyl 35 castor oil. Such co-solvents are typically employed at a level between about 0.01% and about 2% by weight. Viscosity greater than that of simple aqueous solutions may be desirable to decrease variability in dispensing the formulations, to decrease physical separation of components of a suspension or emulsion of formulation, and/or otherwise to improve the formulation. Such viscosity building agents include, for example, polyvinyl alcohol, polyvinyl pyrrolidone, methyl cellulose, hydroxy propyl methylcellulose, hydroxyethyl cellulose, carboxymethyl cellulose, hydroxy propyl cellulose, chondroitin sulfate and salts thereof, hyaluronic acid and salts thereof, and combinations of the foregoing. Such agents are typically employed at a level between about 0.01% and about 2% by weight.

The pharmaceutical compositions may additionally include components to provide sustained release and/or comfort. Such components include high molecular weight, anionic mucomimetic polymers, gelling polysaccharides, and finely-divided drug carrier substrates.

The pharmaceutical composition may be intended for intravenous use. The pharmaceutically acceptable excipient can include buffers to adjust the pH to a desirable range for intravenous use. Many buffers including salts of inorganic acids such as phosphate, borate, and sulfate are known.

In an aspect, provided is a cell containing a compound including a nucleic acid (A) (e.g. formula (I), (II), (III), (III-a), (III-b), (III-c), (III-d), (III-e), (III-f), (III-g), (IV), or (IV-a)) described above including in any aspects, embodiments, claims, figures (e.g., FIGS. 1A-1O, tables (e.g., Tables 1-5 and A-H), examples, or schemes (e.g., Schemes I, II, III, and IV). In embodiments, the cell contains a compound including an oligonucleotide. In embodiments, the cell contains a compound including a double-stranded oligonucleotide. In embodiments, the cell contains a compound including a single-stranded oligonucleotide.

In embodiments, the cell containing the compound including a nucleic acid (A) may include, but be not limited to, a fibroblast cell, a kidney cell, an endothelial cell, an adipose cell, a neuronal cell, a muscle cell, a hepatocyte cell, a T lymphocyte, and a B lymphocyte. In embodiments, the cell containing the compound including a nucleic acid (A) may include, but be not limited to, a human umbilical vein endothelial cell, NIH3T3 cell, RAW264.7 cell, a HEK293 cell or SH-SY5Y cell.

### Methods and Use

In an aspect, provided is a method including contacting a cell with a compound as described herein. In embodiments, the method includes contacting a cell with one or more compounds (e.g. formula (I), (II), (III), (III-a), (III-b), (III-c), (III-d), (III-e), (III-f), (III-g), (IV), or (IV-a)) described above including in any aspects, embodiments, claims, figures (e.g., FIGS. 1A-1O, tables (e.g., Tables 1-5 and A-H), examples, or schemes (e.g., Schemes I, II, III, and IV). In embodiments, the contacting occurs in vitro. In embodiments, the contacting occurs ex vivo. In embodiments, the contacting occurs in vivo.

In an aspect, provided is a method administering to a subject a compound as described herein. In embodiments, the method includes administering to a subject one or more compounds (e.g. formula (I), (II), (III), (III-a), (III-b), (III-c), (III-d), (III-e), (III-f), (III-g), (IV), or (IV-a)) described above including in any aspects, embodiments, claims, figures (e.g., FIGS. 1A-1O, tables (e.g., Tables 1-5 and A-H), examples, or schemes (e.g., Schemes I, II, III, and IV). In embodiments, the subject has a disease or disorder of the eye, liver, kidney, heart, adipose tissue, lung, muscle or spleen.

In embodiments related to administration in vivo or to a subject, the administration is systemic administration, which may include, without limitation, subcutaneous administration, intravenous administration, intramuscular administration, and oral administration. In embodiments related to administration in vivo or to a subject, the administration is local administration, which may include, without limitation, intravitreal administration, intrathecal administration, and intraventricular administration.

In an aspect, provided is a use of a compound as described herein in a therapy. In an aspect, provided is a use of a compound as described herein in the preparation of a medicament.

In an aspect, provided is a method of introducing a compound into a cell within a subject. In embodiments, the method includes administering to said subject a compound as described herein.

### EMBODIMENTS

Embodiment 1. A compound comprising a nucleic acid (A) covalently bonded to a half-life extension motif (HLEM).

Embodiment 2. The compound of Embodiment 1, wherein the compound has a formula (I)

(HLEM)_{z}-A (I),

wherein z is an integer from 1 to 5.

Embodiment 3. The compound of Embodiment 1, wherein the nucleic acid is covalently bonded to an uptake motif (UM).

Embodiment 4. The compound of Embodiment 3, wherein the compound has a formula (II):

(HLEM)_{z}-A-(UM)ₜ (II),

wherein t is an integer from 1 to 5.

Embodiment 5. The compound of one of Embodiments 1 to 4, wherein the half-life extension motif has the structure:

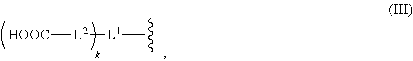

wherein:

L¹ is independently a covalent linker;

L² is independently an unsubstituted alkylene; and

k is an integer from 1 to 5.

Embodiment 6. The compound of Embodiment 5, wherein

L¹ is L^{1A}-L^{1B}-L^{1C}-L^{1D}-L^{1E};

L² is an unsubstituted C₂-C₂₂ alkylene;

L^{1A}, L^{1B}, L^{1C}, L^{1D}, and L^{1E} are independently a

bond, —N(R²⁰)—, —O—, —S—, —C(O)—, —N(R²⁰)C(O)—, —C(O)N(R²¹)—, —N(R²⁰)C(O)N(R²¹)—, —C(O)O—, —OC(O)—, —N(R²⁰)C(O)O—, —OC(O)N(R²¹)—, —OPO₂—O—, —O—P(O)(S)—O—, —O—P(O)(R²²)—O—, —O—P(S)(R²²)—O—, —O—P(O)(NR²⁰R²¹)—N—, —O—P(S)(NR²⁰R²¹)—N—, —O—P(O)(NR²⁰R²¹)—O—, —O—P(S)(NR²⁰R²¹)—O—, —P(O)(NR²⁰R²¹)—N—, —P(S)(NR²⁰R²¹)—N—, —P(O)(NR²⁰R²¹)—O—, —P(S)(NR²⁰R²¹)—O—, —S—S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and

each R²⁰, R²¹ and R²² is independently hydrogen or unsubstituted C₁-C₁₀ alkyl.

Embodiment 7. The compound of one of Embodiments 5 to 6, wherein the largest dimension of L¹ is less than 200 angstroms.

Embodiment 8. The compound of one of Embodiments 5 to 6, wherein the longest linear atomic path L¹ is 1 to 60 atoms in length.

Embodiment 9. The compound of one of Embodiments 5 to 6, wherein the largest dimension of each of L^{1A}, L^{1B}, L^{1C}, L^{1D}, and L^{1E} is independently less than 40 angstroms.

Embodiment 10. The compound of one of Embodiments 5 to 6, wherein each of L^{1A}, L^{1B}, L^{1C}, L^{1D}, and L^{1E} is independently 1 to 20 atoms in length.

Embodiment 11. The compound of one of Embodiments 1 to 10, wherein each of R²⁰, R²¹ and R²² is independently hydrogen or unsubstituted C₁-C₃ alkyl.

Embodiment 12. The compound of one of Embodiments 1 to 11, wherein the nucleic acid is an oligonucleotide.

Embodiment 13. The compound of Embodiment 12, wherein one L^{1A} is attached to a 3′ carbon of the oligonucleotide.

Embodiment 14. The compound of Embodiment 12, wherein one L^{1A} is attached to a 3′ nitrogen of the oligonucleotide.

Embodiment 15. The compound of any one of Embodiments 12 to 14, wherein one L^{1A} is attached to a 5′ carbon of the oligonucleotide.

Embodiment 16. The compound of any one of Embodiments 12 to 16, wherein one L^{1A} is attached to a 6′ carbon of the oligonucleotide.

Embodiment 17. The compound of any one of Embodiments 12 to 14, wherein one L^{1A} is attached to a 2′ carbon of the oligonucleotide.

Embodiment 18. The compound of one of Embodiments 12 to 14, wherein one L^{1A} is attached to a nucleobase of the oligonucleotide.

Embodiment 19. The compound of one of Embodiments 5 to 18, wherein L^{1A} is independently —O—, —C(O)—, —C(O)O—, —OC(O)—, —OPO₂—O—, —O—P(O)(S)—O—, —O—P(O)(CH₃)—O—, —O—P(S)(CH₃)—O—, —O—P(O)(N(CH₃)₂)—N—, —O—P(O)(N(CH₃)₂)—O—, —O—P(S)(N(CH₃)₂)—N—, —O—P(S)(N(CH₃)₂)—O—, —P(O)(N(CH₃)₂)—N—, —P(O)(N(CH₃)₂)—O—, —P(S)(N(CH₃)₂)—N—, —P(S)(N(CH₃)₂)—O—, substituted or unsubstituted alkylene, or substituted or unsubstituted heteroalkylene.

Embodiment 20. The compound of one of Embodiments 6 to 19, wherein L^{1A} is independently

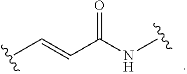

Embodiment 21. The compound of one of Embodiments 6 to 19, wherein L^{1A} is independently —OPO₂—O— or —OP(O)(S)—O—.

Embodiment 22. The compound of one of Embodiments 6 to 19, wherein L^{1A} is independently —O—.

Embodiment 23. The compound of any one of Embodiments 6 to 19, wherein L^{1A} is independently —C(O)—.

Embodiment 24. The compound of any one of Embodiments 6 to 19, wherein L^{1A} is independently —O—P(O)(N(CH₃)₂)—N—.

Embodiment 25. The compound of one of Embodiments 6 to 22, wherein L^{1B} is independently substituted or unsubstituted alkylene or substituted or unsubstituted heteroalkylene.

Embodiment 26. The compound of one of Embodiments 6 to 22, wherein L^{1B} is independently -L¹⁰-NH—C(O)— or -L¹⁰-C(O)—NH—, wherein L¹⁰ is substituted or unsubstituted alkylene.

Embodiment 27. The compound of one of Embodiments 6 to 22, wherein L^{1B} is independently

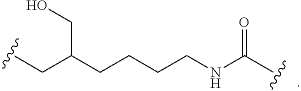

Embodiment 28/ The compound of one of Embodiments 6 to 22, wherein L^{1B} is independently

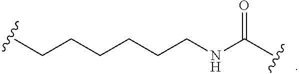

Embodiment 29. The compound of one of Embodiments 6 to 22, wherein L^{1B} is independently

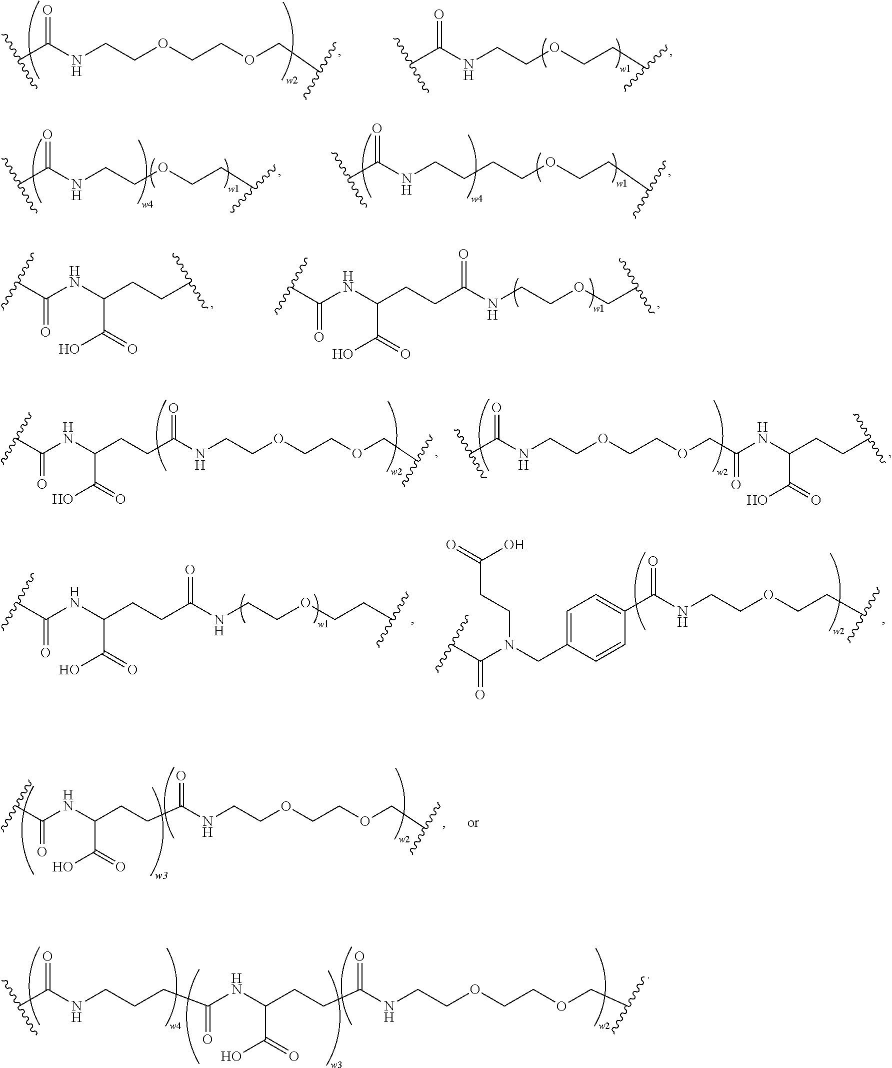

w1 is an integer from 0 to 10;

w2 is an integer from 0 to 5;

w3 is an integer from 0 to 5; and

w4 is an integer from 0 to 5.

Embodiment 30. The compound of one of Embodiments 6 to 22, wherein L^{1B} is independently

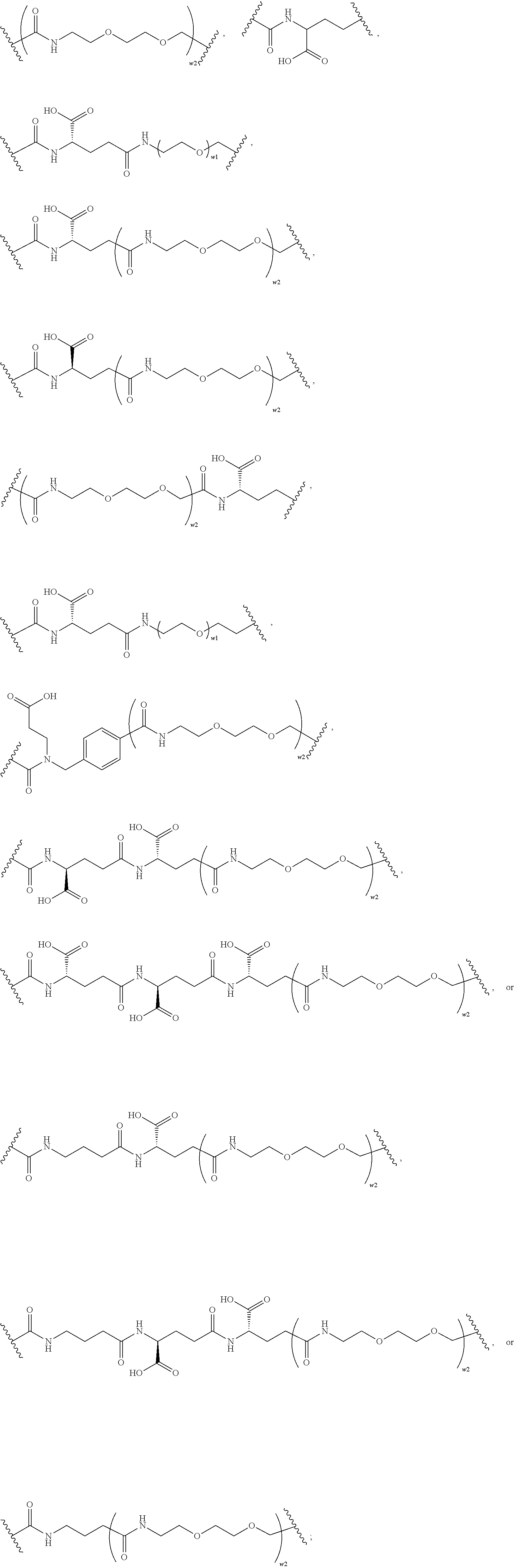

w1 is an integer from 0 to 10; and

w2 is an integer from 0 to 5.

Embodiment 31. The compound of one of Embodiments 6 to 22, wherein L^{1B} is independently

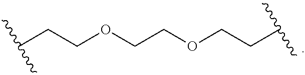 [SMILES: *CCOCCOCC*]

Embodiment 32. The compound of one of Embodiments 6 to 22, wherein L^{1B} is independently

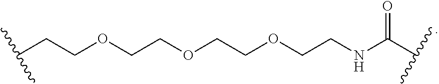 [SMILES: *CCOCCOCCOCCNC(*)=O]

Embodiment 33. The compound of one of Embodiments 6 to 32, wherein -L^{1A}-L^{1B}- is independently —O-L¹⁰-NH—C(O)— or —O-L¹⁰-C(O)—NH—, wherein L¹⁰ is independently substituted or unsubstituted alkylene, substituted or unsubstituted heteroalkylene, or substituted or unsubstituted heteroalkenylene.

Embodiment 34. The compound of one of Embodiments 6 to 32, wherein -L^{1A}-L^{1B}- is independently —O-L¹⁰-NH—C(O)—, wherein L¹⁰ is independently substituted or unsubstituted C₅-C₈ alkylene.

Embodiment 35. The compound of one of Embodiments 6 to 32, wherein -L^{1A}-L^{1B}- is independently

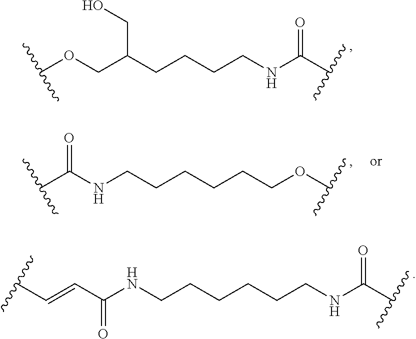 [SMILES: *OCC(CO)CCCCNC(*)=O ; *OCCCCCCNC(*)=O ; */C=C/C(=O)NCCCCCCNC(*)=O]

Embodiment 36. The compound of one of Embodiments 6 to 32, wherein -L^{1A}-L^{1B}- is independently —OPO₂—O-L¹⁰-NH—C(O)—, —OP(O)(S)—O-L¹⁰-NH—C(O)—, —OPO₂—O-L¹⁰-C(O)—NH— or —OP(O)(S)—O-L¹⁰-C(O)—NH—, wherein L¹⁰ is independently substituted or unsubstituted alkylene.

Embodiment 37. The compound of one of Embodiments 6 to 32, wherein -L^{1A}-L^{1B}- is independently —OPO₂—O-L¹⁰-NH—C(O)— or —OP(O)(S)—O-L¹⁰-NH—C(O)—, wherein L¹⁰ is independently substituted or unsubstituted C₅-C₈ alkylene.

Embodiment 38. The compound of one of Embodiments 6 to 37, wherein -L^{1A}-L^{1B}- is independently

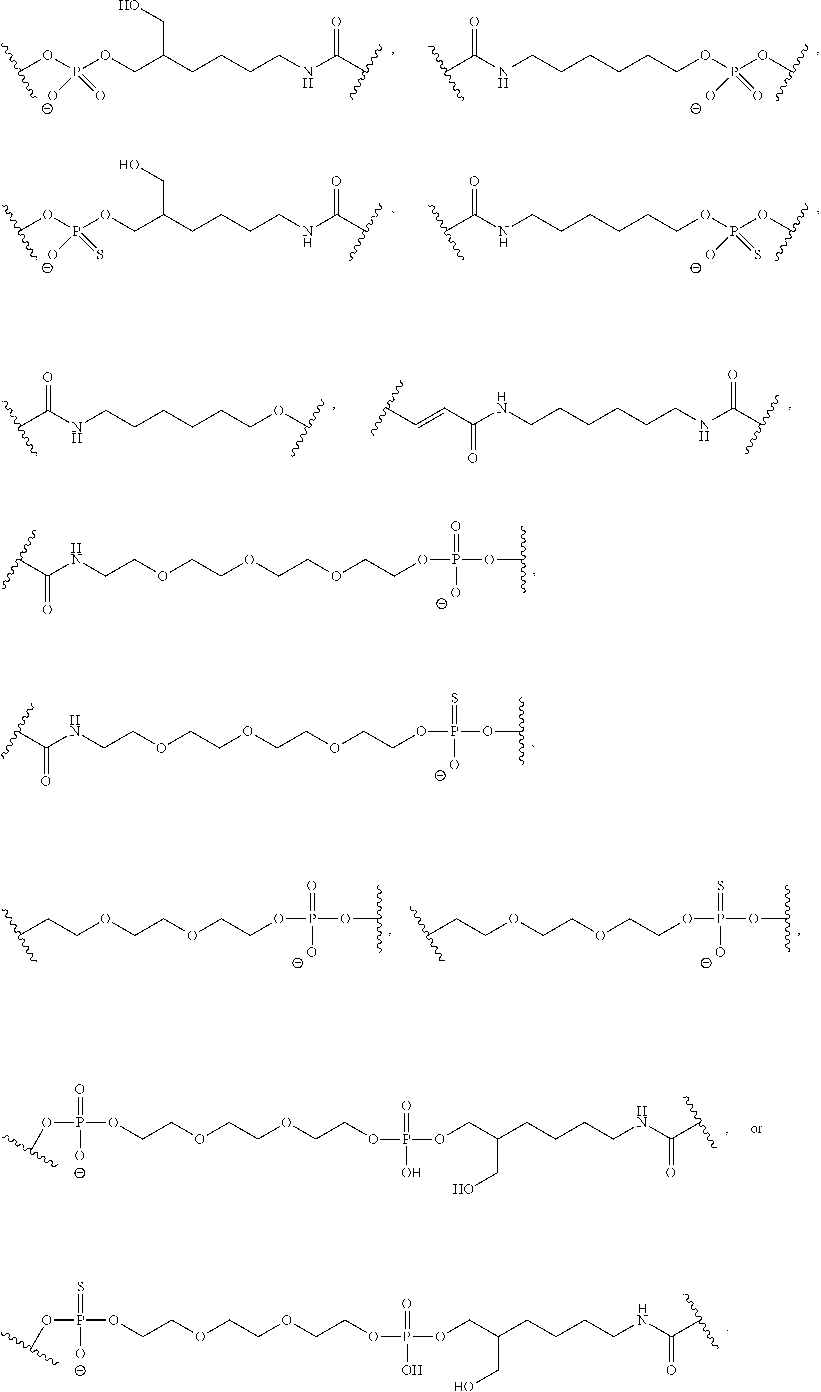

Embodiment 39. The compound of one of Embodiments 12 to 38, wherein an -L^{1A}-L^{1B}- is independently

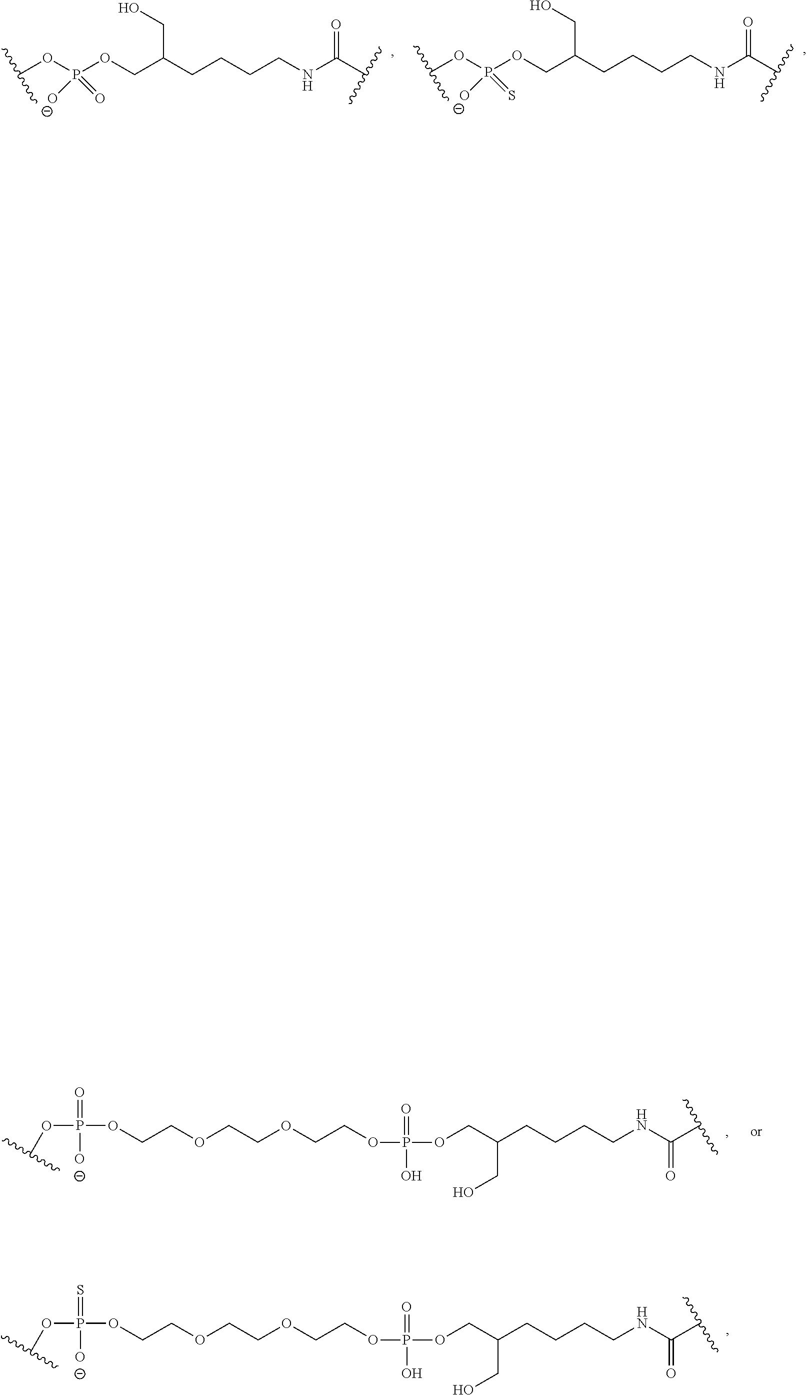 [SMILES: *OP(=O)([O-])OCC(CO)CCCCNC(*)=O ; *OP([O-])(=S)OCC(CO)CCCCNC(*)=O ; *OP(=O)([O-])OCCOCCOCCOP(=O)(O)OCC(CO)CCCCNC(*)=O ; *OP([O-])(=S)OCCOCCOCCOP(=O)(O)OCC(CO)CCCCNC(*)=O]

and is attached to a 3′ carbon of the oligonucleotide.

Embodiment 40. The compound of one of Embodiments 12 to 38, wherein an -L^{1A}-L^{1B}- is independently

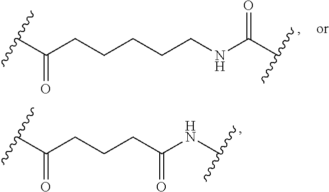 [SMILES: *C(=O)CCCCCNC(*)=O ; *NC(=O)CCCC(*)=O]

and is attached to a 3′ nitrogen of the oligonucleotide.

Embodiment 41. The compound of one of Embodiments 12 to 38, wherein an -L^{1A}-L^{1B}- is independently

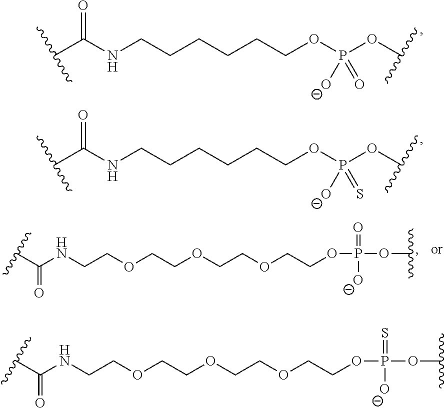 [SMILES: *OP(=O)([O-])OCCCCCCNC(*)=O ; *OP([O-])(=S)OCCCCCCNC(*)=O ; *OP(=O)([O-])OCCOCCOCCOCCNC(*)=O ; *OP([O-])(=S)OCCOCCOCCOCCNC(*)=O]

and is attached to a 5′ carbon of the oligonucleotide.

Embodiment 42. The compound of one of Embodiments 12 to 38, wherein an -L^{1A}-L^{1B}- is independently

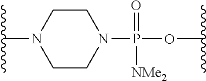 [SMILES: *OP(=O)(N(C)C)N1CCN(*)CC1]

and is attached to a 6′ carbon of the oligonucleotide.

Embodiment 43. The compound of one of Embodiments 12 to 42, wherein an -L^{1A}-L^{1B}- is independently attached to a nucleobase of the oligonucleotide.

Embodiment 44. The compound of one of Embodiments 6 to 43, wherein:

L^{1C} is independently substituted or unsubstituted alkylene, or substituted or unsubstituted heteroalkylene;

L^{1D} is independently a bond, substituted or unsubstituted alkylene, substituted or unsubstituted heteroalkylene, substituted or unsubstituted arylene, or substituted or unsubstituted heteroarylene; and

L^{1E} is independently a bond, substituted or unsubstituted heteroalkylene, or —NHC(O)—.

Embodiment 45. The compound of one of Embodiments 6 to 43, wherein

L^{1C} is independently substituted or unsubstituted C₁-C₁₀ alkylene, or substituted or unsubstituted 2 to 10 membered heteroalkylene;

L^{1D} is independently a bond, substituted or unsubstituted C₁-C₁₀ alkylene, or substituted or unsubstituted 2 to 10 membered heteroalkylene; and

L^{1E} is independently a bond, substituted or unsubstituted 2 to 10 membered heteroalkylene, or —NHC(O)—.

Embodiment 46. The compound of one of Embodiments 6 to 43, wherein:

L^{1C} is independently substituted or unsubstituted C₁-C₇ alkylene, or substituted or unsubstituted 5 to 8 membered heteroalkylene;

L^{1D} is independently a bond, substituted or unsubstituted C₁-C₇ alkylene, or substituted or unsubstituted 5 to 8 membered heteroalkylene; and

L^{1E} is independently a bond, substituted or unsubstituted 6 to 8 membered heteroalkylene, or —NHC(O)—.

Embodiment 47. The compound of one of Embodiments 6 to 43, wherein:

L^{1C} is independently R^{1C}-substituted or unsubstituted C₁-C₇ alkylene, or R^{1C}-substituted or unsubstituted 5 to 8 membered heteroalkylene;

L^{1D} is independently a bond, R^{1D}-substituted or unsubstituted C₁-C₇ alkylene, or R^{1D}-substituted or unsubstituted 5 to 8 membered heteroalkylene; and

L^{1E} is independently a bond, R^{1E}-substituted or unsubstituted 6 to 8 membered heteroalkylene, or —NHC(O)—;

R^{1C} is independently oxo, or -L^{8C}-L^{2C}-R^{8C};

R^{1D} is independently oxo, or -L^{8D}-L^{2D}-R^{8D};

R^{1E} is independently oxo, or -L^{8E}-L^{2E}-R^{8E};

each L^{8C}, L^{8D}, and L^{8E} is independently a bond, substituted or unsubstituted C₁-C₆ alkylene, or substituted or unsubstituted 2 to 6 membered heteroalkylene,

each L^{2C}, L^{2D}, and L^{2E} is independently a bond, or an unsubstituted alkylene; and

each R^{8C}, R^{8D}, and R^{8E} is independently hydrogen, substituted or unsubstituted alkyl, or substituted or unsubstituted heteroalkyl.

Embodiment 48. The compound of one of Embodiments 5 to 47, wherein the half-life extension motif has the structure:

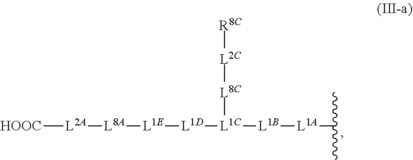

wherein L^{8A} is independently a bond, substituted or unsubstituted alkylene, or substituted or unsubstituted heteroalkylene; and

L^{2A} is independently a bond, or an unsubstituted alkylene.

Embodiment 49. The compound of one of Embodiments 5 to 47, wherein the half-life extension motif has the structure:

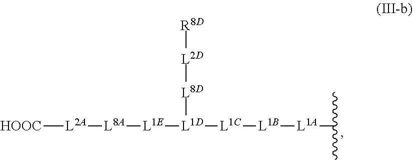

wherein L^{8A} is independently a bond, substituted or unsubstituted alkylene, or substituted or unsubstituted heteroalkylene; and

L^{2A} is independently a bond, or an unsubstituted alkylene.

Embodiment 50. The compound of one of Embodiments 5 to 47, wherein the half-life extension motif has the structure:

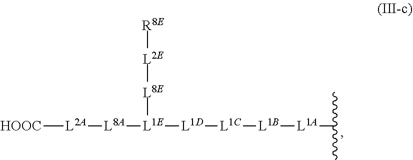

wherein L^{8A} is independently a bond, substituted or unsubstituted alkylene, or substituted or unsubstituted heteroalkylene; and

L^{2A} is independently a bond, or an unsubstituted alkylene.

Embodiment 51. The compound of one of Embodiments 5 to 47, wherein the half-life extension motif has the structure:

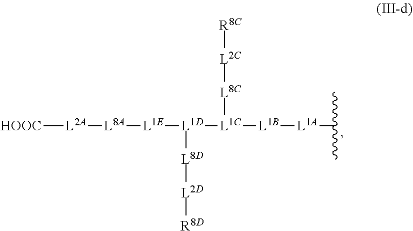

wherein L^{8A} is independently a bond, substituted or unsubstituted alkylene, or substituted or unsubstituted heteroalkylene; and

L^{2A} is independently a bond, or an unsubstituted alkylene.

Embodiment 52. The compound of one of Embodiments 5 to 47, wherein the half-life extension motif has the structure:

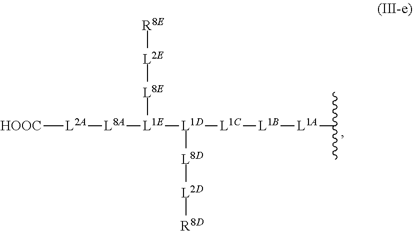

wherein L^{8A} is independently a bond, substituted or unsubstituted alkylene, or substituted or unsubstituted heteroalkylene; and

L^{2A} is independently a bond, or an unsubstituted alkylene.

Embodiment 53. The compound of one of Embodiments 5 to 47, wherein the half-life extension motif has the structure:

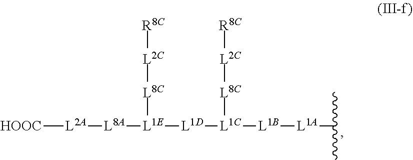

wherein L^{8A} is independently a bond, substituted or unsubstituted alkylene, or substituted or unsubstituted heteroalkylene; and

L^{2A} is independently a bond, or an unsubstituted alkylene.

Embodiment 54. The compound of one of Embodiments 5 to 47, wherein the half-life extension motif has the structure:

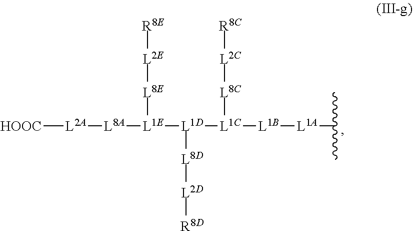

wherein L^{8A} is independently a bond, substituted or unsubstituted alkylene, or substituted or unsubstituted heteroalkylene; and

L^{2A} is independently a bond, or an unsubstituted alkylene.

Embodiment 55. The compound of one of Embodiments 5 to 54, wherein:

R^{1C} is independently —NHC(O)-L^{2C}-R^{8C};

L^{2C} is independently a bond or an unsubstituted C₂-C₂₂ alkylene; and

R^{8C} is independently hydrogen, unsubstituted C₁-C₃ alkyl, or —COOH.

Embodiment 56. The compound of one of Embodiments 5 to 54, wherein:

R^{1C} is independently —NHC(O)-L^{2C}-R^{8C};

L^{2C} is independently a bond; and

R^{8C} is independently unsubstituted C₁-C₃ alkyl.

Embodiment 57. The compound of one of Embodiments 5 to 54, wherein:

R^{1C} is independently —NHC(O)-L^{2C}-R^{8C};

L^{2C} is independently an unsubstituted C₁₀-C₂₂ alkylene; and

R^{8C} is independently an unsubstituted C₁-C₃ alkyl, or —COOH.

Embodiment 58. The compound of one of Embodiments 5 to 54, wherein:

R^{1D} is independently —NHC(O)-L^{2D}-R^{8D};

L^{2D} is independently a bond or an unsubstituted C₂-C₂₂ alkylene;

R^{8D} is independently hydrogen, unsubstituted C₁-C₃ alkyl, or —COOH.

Embodiment 59. The compound of one of Embodiments 5 to 54, wherein:

R^{1E} is independently —NHC(O)-L^{2E}-R^{8E};

L^{2E} is independently a bond or an unsubstituted C₂-C₂₂ alkylene;

R^{8E} is independently hydrogen, unsubstituted C₁-C₃ alkyl, or COOH.

Embodiment 60. The compound of one of Embodiments 5 to 59, wherein:

L^{1C} is independently R^{1C}-substituted or unsubstituted C₃-C₇ alkylene;

L^{1D} is independently a bond, or an unsubstituted arylene;

L^{1E} is independently R^{1E}-substituted or unsubstituted 5 to 8 membered heteroalkylene or —NHC(O)—.

Embodiment 61. The compound of one of Embodiments 5 to 59, wherein

L^{1C} is independently R^{1C}-substituted or unsubstituted 5 to 8 membered heteroalkylene;

L^{1D} is independently a bond, or R^{1D}-substituted or unsubstituted 5 to 8 membered heteroalkylene;

L^{1E} is independently R^{1E}-substituted or unsubstituted 5 to 8 membered heteroalkylene or —NHC(O)—;

each R^{1C}, R^{1D}, or R^{1E} is independently oxo, or —COOH.

Embodiment 62. The compound of one of Embodiments 5 to 59, wherein:

L^{1C} is independently R^{1C}-substituted C₂-C₅ alkyl;

L^{1D} is independently an unsubstituted phenylene, or an unsubstituted biphenylene; and

L^{1E} is independently R^{1E}-substituted or unsubstituted 5 to 8 membered heteroalkylene or —NHC(O)—; and

R^{1C} is independently —NHC(O)-L^{2C}-R^{8C};

L^{2C} is independently a bond, or an unsubstituted C₁₀-C₂₂ alkylene;

R^{8C} is independently an unsubstituted C₁-C₃ alkyl, or —COOH; and

R^{1E} is oxo.

Embodiment 63. The compound of one of Embodiments 5 to 59, wherein:

L^{1C} is independently R^{1C}-substituted or unsubstituted ethylene or n-pentylene;

L^{1D} is independently a bond;

L^{1E} is independently —NHC(O)—;

R^{1C} is independently —NHC(O)-L^{2C}-R^{8C};

L^{2C} is independently a bond or an unsubstituted C₁₀-C₂₂ alkylene; and

R^{8C} is independently an unsubstituted C₁-C₃ alkyl, or —COOH.

Embodiment 64. The compound of one of Embodiments 5 to 59, wherein:

L^{1C} is independently R^{1C}-substituted or unsubstituted n-pentylene;

L^{1D} is independently oxo-substituted or unsubstituted 5 to 8 membered heteroalkylene;

L^{1E} is independently —NHC(O)—; and

R^{1C} is independently —NHC(O)-L^{2C}-R^{8C};

L^{2C} is independently a bond or an unsubstituted C₁₀-C₂₂ alkylene; and

R^{8C} is independently an unsubstituted C₁-C₃ alkyl, or —COOH.

Embodiment 65. The compound of one of Embodiments 5 to 59, wherein:

L^{1C} is independently R^{1C}-substituted methylene;

L^{1D} is independently a bond; and

L^{1E} is independently —NHC(O)—; and

R^{1C} is independently -L^{8C}-L^{2C}-R^{8C};

L^{8C} is independently an unsubstituted C₁-C₆ alkylene or oxo-substituted 2 to 12 membered heteroalkylene;

L^{2C} is independently a bond; and

R^{8C} is independently an unsubstituted C₁-C₆ alkyl or oxo-substituted 2 to 12 membered heteroalkyl.

Embodiment 66. The compound of Embodiment 65, wherein R^{8C} is independently an unsubstituted C₁-C₆ alkyl or oxo- and C₁-C₁₅ alkyl-substituted 2 to 12 membered heteroalkyl.

Embodiment 67. The compound of one of Embodiments 5 to 66, wherein L¹ is:

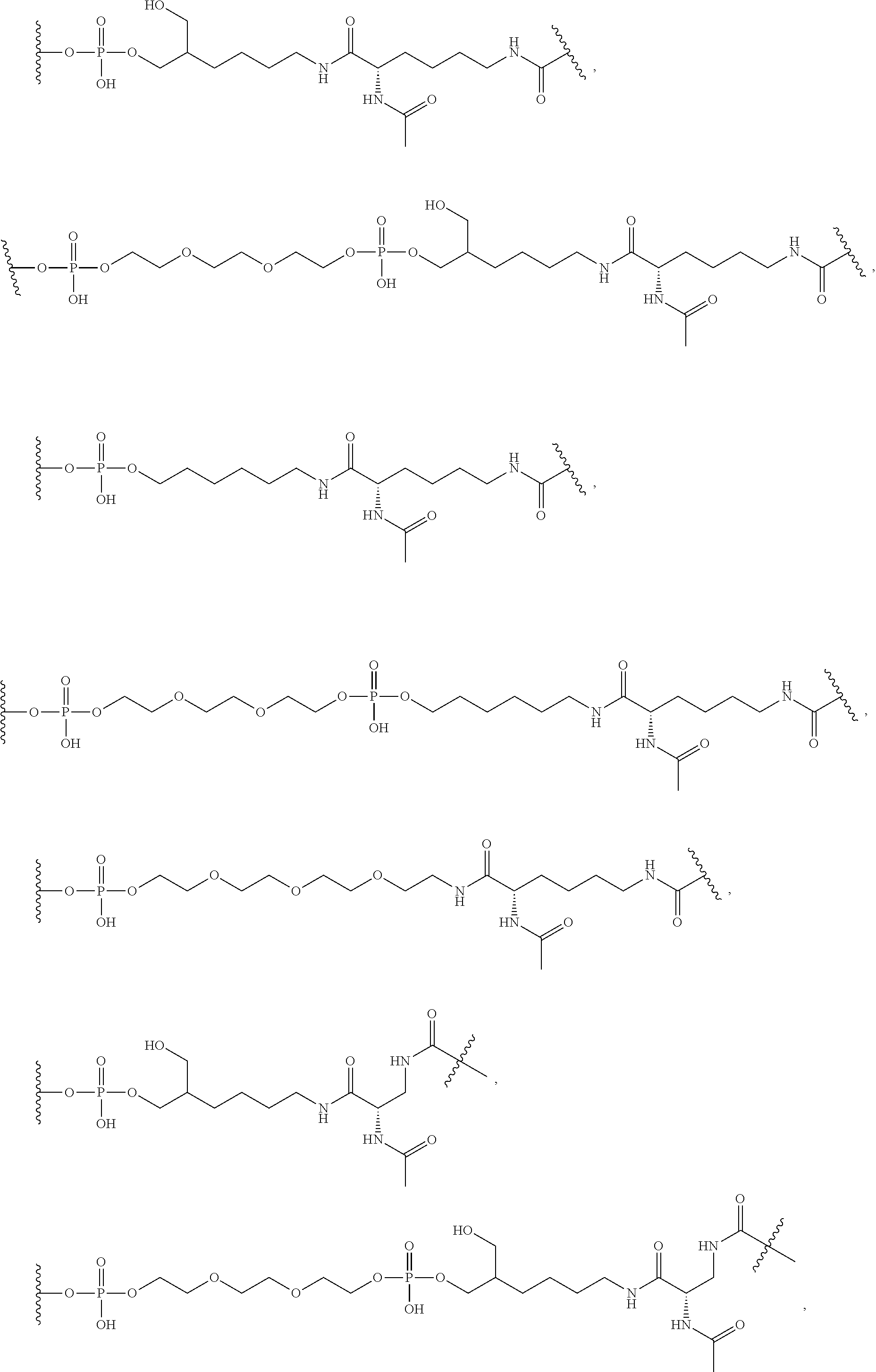

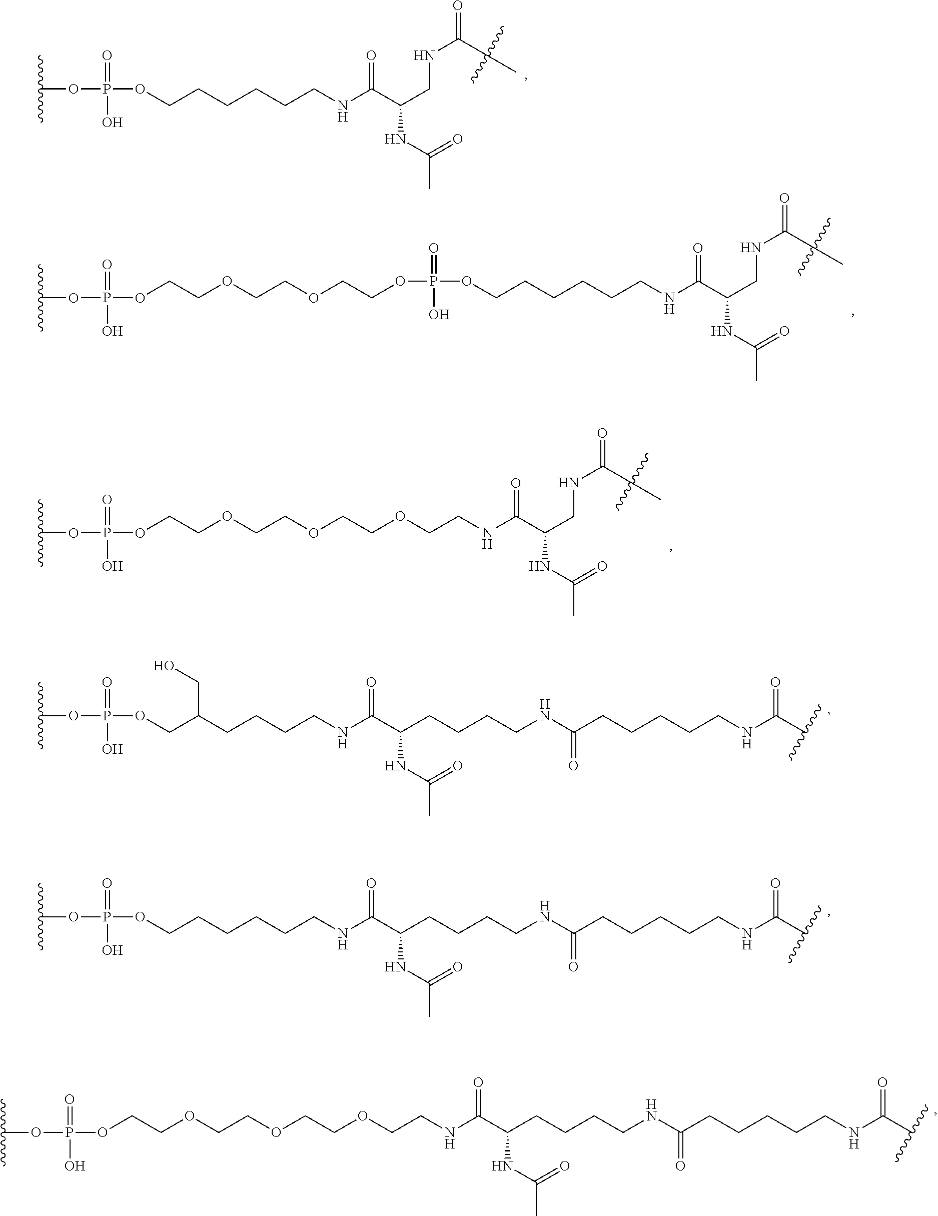 [SMILES: *OP(=O)(O)OCCCCCCNC(=O)[C@H](CNC(*)=O)NC(C)=O ; *OP(=O)(O)OCCOCCOCCOP(=O)(O)OCCCCCCNC(=O)[C@H](CNC(*)=O)NC(C)=O ; *OP(=O)(O)OCCOCCOCCOCCNC(=O)[C@H](CNC(*)=O)NC(C)=O ; *OP(=O)(O)OCC(CO)CCCCNC(=O)[C@H](CCCCNC(=O)CCCCCNC(*)=O)NC(C)=O ; *OP(=O)(O)OCCCCCCNC(=O)[C@H](CCCCNC(=O)CCCCCNC(*)=O)NC(C)=O ; *OP(=O)(O)OCCOCCOCCOCCNC(=O)[C@H](CCCCNC(=O)CCCCCNC(*)=O)NC(C)=O]

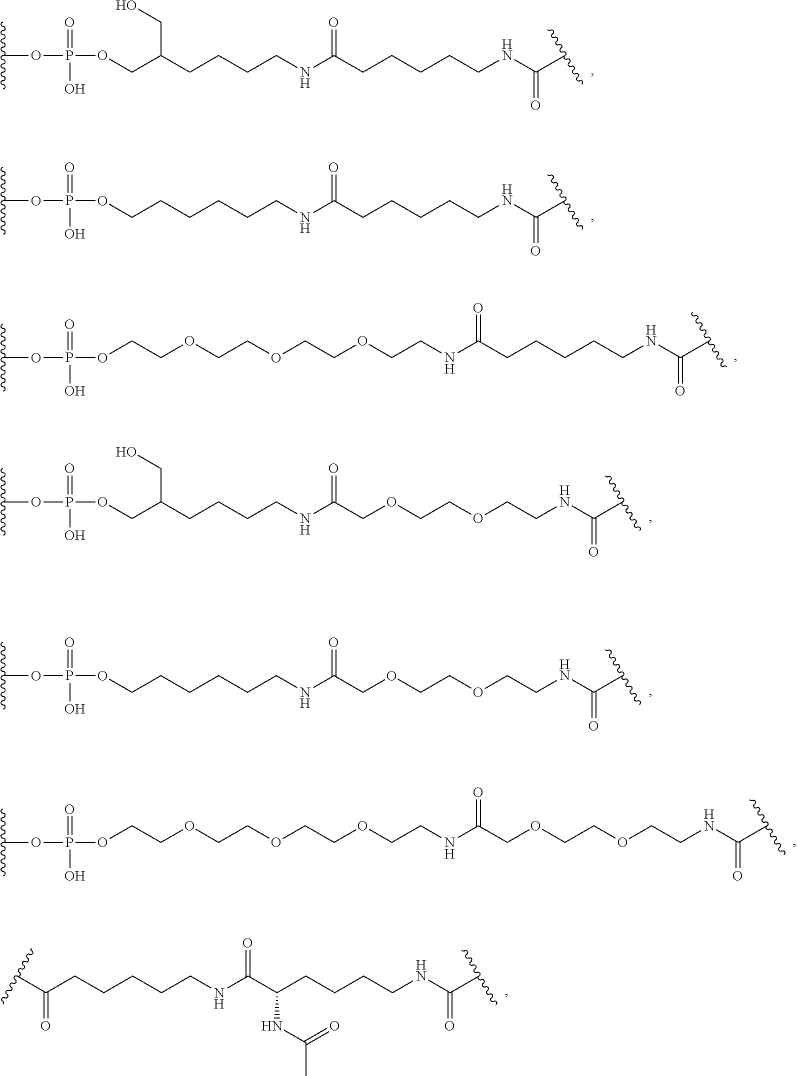

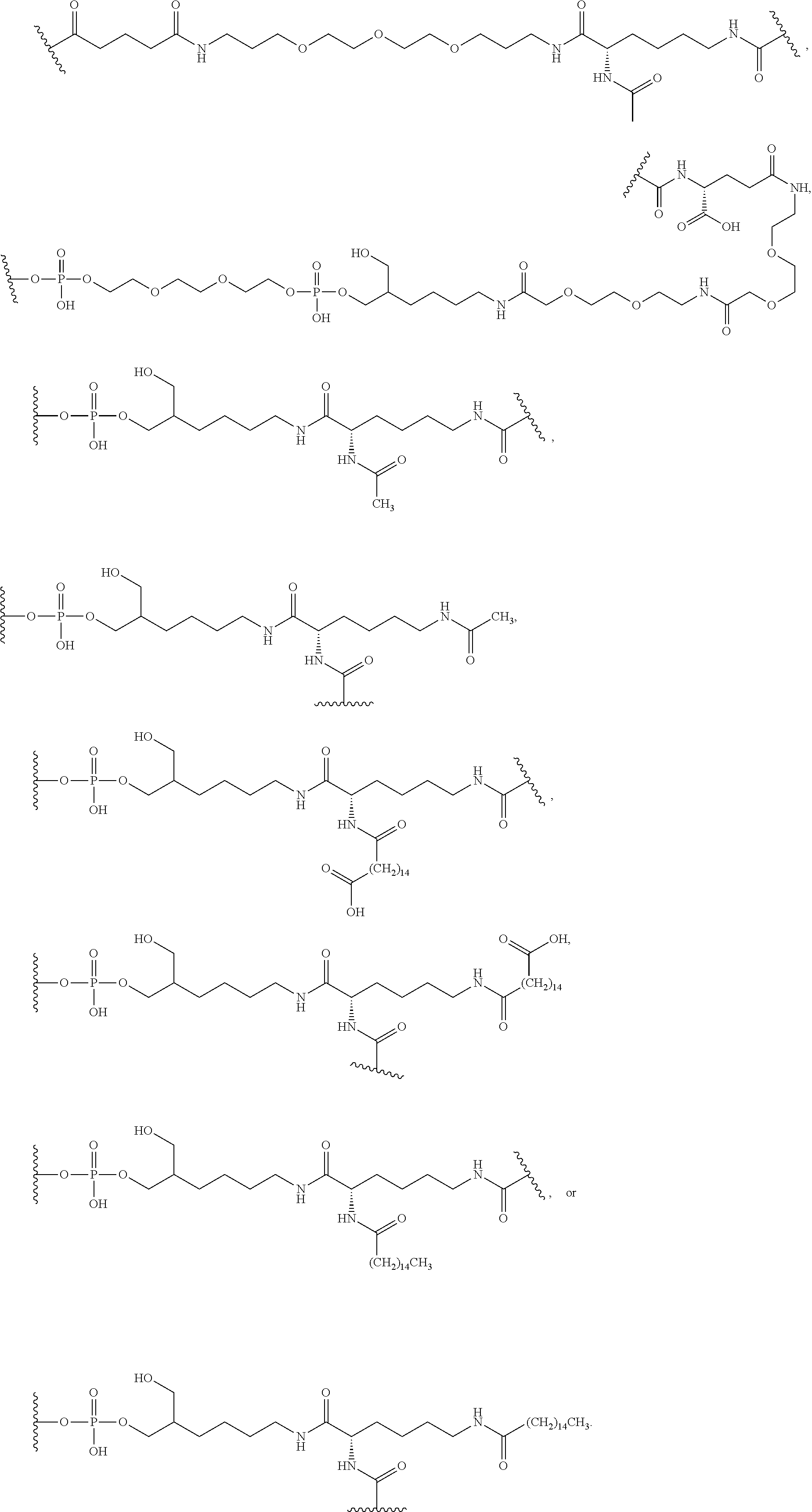

Embodiment 68. The compound of one of Embodiments 5 to 67, wherein each L², L^{2A}, L^{2C}, L^{2D}, or L^{2E} is independently an unsubstituted C₂-C₂₂ alkylene.

Embodiment 69. The compound of one of Embodiments 5 to 67, wherein each L², L^{2A}, L^{2C}, L^{2D}, or L^{2E} is independently an unsubstituted C₅-C₂₂ alkylene.

The compound of one of Embodiments 5 to 67, wherein each L², L^{2A}, L^{2C}, L^{2D}, or L^{2E} is independently an unsubstituted C₁₀-C₂₂ alkylene.

Embodiment 71. The compound of one of Embodiments 5 to 67, wherein each L², L^{2A}, L^{2C}, L^{2D}, or L^{2E} is an unsubstituted C₁₂-C₂₂ alkylene.

Embodiment 72. The compound of one of Embodiments 5 to 67, wherein each L², L^{2A}, L^{2C}, L^{2D}, or L^{2E} is an unsubstituted C₁₂-C₁₈ alkylene.

Embodiment 73. The compound of one of Embodiments 5 to 67, wherein each L², L^{2A}, L^{2C}, L^{2D}, or L^{2E} is an unsubstituted C₁₂-C₁₆ alkylene.

Embodiment 74. The compound of one of Embodiments 5 to 67, wherein each L², L^{2A}, L^{2C}, L^{2D}, or L^{2E} is an unsubstituted C₁₄-C₁₅ alkylene.

Embodiment 75. The compound of one of Embodiments 5 to 67, wherein each L², L^{2A}, L^{2C}, L^{2D}, or L^{2E} is an unsubstituted unbranched C₁₀-C₂₂ alkylene.

Embodiment 76. The compound of one of Embodiments 5 to 67, wherein each L², L^{2A}, L^{2C}, L^{2D}, or L^{2E} is an unsubstituted unbranched C₁₂-C₂₂ alkylene.

Embodiment 77. The compound of one of Embodiments 5 to 67, wherein each L², L^{2A}, L^{2C}, L^{2D}, or L^{2E} is an unsubstituted unbranched C₁₂-C₁₈ alkylene.

Embodiment 78. The compound of one of Embodiments 5 to 67, wherein each L², L^{2A}, L^{2C}, L^{2D}, or L^{2E} is an unsubstituted unbranched C₁₂-C₁₆ alkylene.

Embodiment 79. The compound of one of Embodiments 5 to 67, wherein each L², L^{2A}, L^{2C}, L^{2D}, or L^{2E} is an unsubstituted unbranched C₁₄-C₁₅ alkylene.

Embodiment 80. The compound of one of Embodiments 5 to 67, wherein each L², L^{2A}, L^{2C}, L^{2D}, or L^{2E} is an unsubstituted unbranched saturated C₁₀-C₂₂ alkylene.

Embodiment 81. The compound of one of Embodiments 5 to 67, wherein each L², L^{2A}, L^{2C}, L^{2D}, or L^{2E} is an unsubstituted unbranched saturated C₁₂-C₂₂ alkylene.

Embodiment 82. The compound of one of Embodiments 5 to 67, wherein each L², L^{2A}, L^{2C}, L^{2D}, or L^{2E} is an unsubstituted unbranched saturated C₁₂-C₁₈ alkylene.

Embodiment 83. The compound of one of Embodiments 5 to 67, wherein each L², L^{2A}, L^{2C}, L^{2D}, or L^{2E} is an unsubstituted unbranched saturated C₁₂-C₁₆ alkylene.

Embodiment 84. The compound of one of Embodiments 5 to 67, wherein each L², L^{2A}, L^{2C}, L^{2D}, or L^{2E} is an unsubstituted unbranched saturated C₁₄-C₁₅ alkylene.

Embodiment 85. The compound of one of Embodiments 5 to 84, comprising from one to five optionally different half-life extension motifs.

Embodiment 86. The compound of one of Embodiments 5 to 84, comprising only one half-life extension motif.

Embodiment 87. The compound of one of Embodiments 3 to 86, wherein the uptake motif independently has the structure:

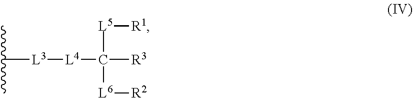

wherein:

L³ and L⁴ are independently a bond, —N(R²³)—, —O—, —S—, —C(O)—, —N(R²³)C(O)—, —C(O)N(R²⁴)—, —N(R²³)C(O)N(R²⁴)—, —C(O)O—, —OC(O)—, —N(R²³)C(O)O—, —OC(O)N(R²⁴)—, —OPO₂—O—, —O—P(O)(S)—O—, —O—P(O)(R²⁵)—O—, —O—P(S)(R²⁵)—O—, —O—P(O)(NR²³R²⁴)—N—, —O—P(S)(NR²³R²⁴)—N—, —O—P(O)(NR²³R²⁴)—O—, —O—P(S)(NR²³R²⁴)—O—, —P(O)(NR²³R²⁴)—N—, —P(S)(NR²³R²⁴)—N—, —P(O)(NR²³R²⁴)—O—, —P(S)(NR²³R²⁴)—O—, —S—S—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene;

L⁵ is -L^{5A}-L^{5B}-L^{5C}-L^{5D}-L^{5E}-;

L⁶ is -L^{6A}-L^{6B}-L^{6C}-L^{6D}-L^{6E}-;

R¹ and R² are independently unsubstituted C₁-C₂₅ alkyl, wherein at least one of R¹ and R² is unsubstituted C₉-C₁₉ alkyl;

R³ is hydrogen, —NH₂, —OH, —SH, —C(O)H, —C(O)NH₂, —NHC(O)H,

—NHC(O)OH, —NHC(O)NH₂, —C(O)OH, —OC(O)H, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

L^{5A}, L^{5B}, L^{5C}, L^{5D}, L^{5E}, L^{6A}, L^{6B}, L^{6C}, L^{6D}, and L^{6E} are independently a

bond, —NH—, —O—, —S—, —C(O)—, —NHC(O)—, —NHC(O)NH—, —C(O)O—, —OC(O)—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene; and

each R²³, R²⁴ and R²⁵ is independently hydrogen or unsubstituted C₁-C₁₀ alkyl.

Embodiment 88. The compound of one of Embodiments 4 to 87, wherein t is 1.

Embodiment 89. The compound of one of Embodiments 4 to 87, wherein t is 2.

Embodiment 90. The compound of one of Embodiments 4 to 87, wherein t is 3.

Embodiment 91. The compound of one of Embodiments 4 to 90, wherein each of R²³, R²⁴ and R²⁵ is independently hydrogen or unsubstituted C₁-C₃ alkyl.

Embodiment 92. The compound of one of Embodiments 87 to 90, wherein one L³ is attached to a 3′ carbon of the oligonucleotide.

Embodiment 93. The compound of one of Embodiments 87 to 90, wherein one L³ is attached to a 3′ nitrogen of the oligonucleotide.

Embodiment 94. The compound of one of Embodiments 87 to 90, wherein one L³ is attached to a 5′ carbon of the oligonucleotide.

Embodiment 95. The compound of one of Embodiments 87 to 90, wherein one L³ is attached to a 6′ carbon of the oligonucleotide.

Embodiment 96. The compound of one of Embodiments 87 to 90, wherein one L³ is attached to a nucleobase of the oligonucleotide.

Embodiment 97. The compound of one of Embodiments 87 to 96, wherein L³ and L⁴ are independently a bond, —NH—, —O—, —C(O)—, —C(O)O—, —OC(O)—, —OPO₂—O—, —O—P(O)(S)—O—, —O—P(O)(CH₃)—O—, —O—P(S)(CH₃)—O—, —O—P(O)(N(CH₃)₂)—N—, —O—P(O)(N(CH₃)₂)—O—, —O—P(S)(N(CH₃)₂)—N—, —O—P(S)(N(CH₃)₂)—O—, —P(O)(N(CH₃)₂)—N—, —P(O)(N(CH₃)₂)—O—, —P(S)(N(CH₃)₂)—N—, —P(S)(N(CH₃)₂)—O—, substituted or unsubstituted alkylene or substituted or unsubstituted heteroalkylene.

Embodiment 98. The compound of one of Embodiments 87 to 97, wherein L³ is independently

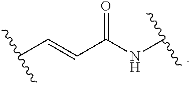 [SMILES: */C=C/C(=O)N*]

Embodiment 99. The compound of one of Embodiments 87 to 97, wherein L³ is independently —OPO₂—O— or —OP(O)(S)—O—.

Embodiment 100. The compound of one of Embodiments 87 to 98, wherein L³ is independently —O—.

Embodiment 101. The compound of any one of Embodiments 87 to 97, wherein L³ is independently —C(O)—.

Embodiment 102. The compound of any one of Embodiments 87 to 97, wherein L³ is independently —O—P(O)(N(CH₃)₂)—N—.

Embodiment 103. The compound of one of Embodiments 87 to 102, wherein L⁴ is independently substituted or unsubstituted alkylene or substituted or unsubstituted heteroalkylene.

Embodiment 104. The compound of one of Embodiments 87 to 102, wherein L⁴ is independently -L⁷-NH—C(O)— or -L⁷-C(O)—NH—, wherein L⁷ is substituted or unsubstituted alkylene.

Embodiment 105. The compound of one of Embodiments 87 to 104, wherein L⁴ is independently

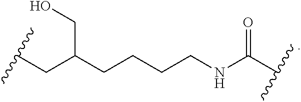 [SMILES: *CC(CO)CCCCNC(*)=O]

Embodiment 106. The compound of one of Embodiments 87 to 104, wherein L⁴ is independently

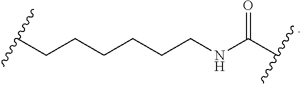 [SMILES: *CCCCCCNC(*)=O]

Embodiment 107. The compound of one of Embodiments 87 to 106, wherein -L³-L⁴- is independently —O-L⁷-NH—C(O)— or —O-L⁷-C(O)—NH—, wherein L⁷ is independently substituted or unsubstituted alkylene, substituted or unsubstituted heteroalkylene, or substituted or unsubstituted heteroalkenylene.

Embodiment 107. The compound of Embodiment 107, wherein -L³-L⁴- is independently —O-L⁷-NH—C(O)—, wherein L⁷ is independently substituted or unsubstituted C₅-C₈ alkylene.

Embodiment 109. The compound of Embodiment 108, wherein -L³-L⁴- is independently

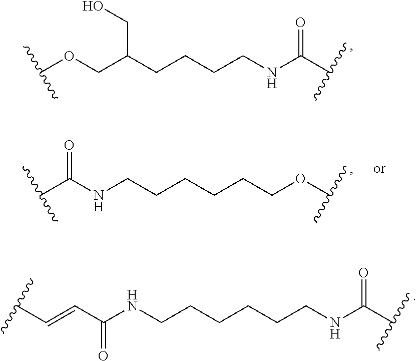 [SMILES: *OCC(CO)CCCCNC(*)=O ; *OCCCCCCNC(*)=O ; */C=C/C(=O)NCCCCCCNC(*)=O]

Embodiment 110. The compound of one of Embodiments 87 to 97, wherein -L³-L⁴- is independently —OPO₂—O-L⁷-NH—C(O)—, —OP(O)(S)—O-L⁷-NH—C(O)—, —OPO₂—O-L⁷-C(O)—NH— or —OP(O)(S)—O-L⁷-C(O)—NH—, wherein L⁷ is independently substituted or unsubstituted alkylene.

Embodiment 111. The compound of Embodiment 110, wherein -L³-L⁴- is independently —OPO₂—O-L⁷-NH—C(O)— or —OP(O)(S)—O-L⁷-NH—C(O)—, wherein L⁷ is independently substituted or unsubstituted C₅-C₈ alkylene.

Embodiment 112. The compound of Embodiment 111, wherein -L³-L⁴- is independently

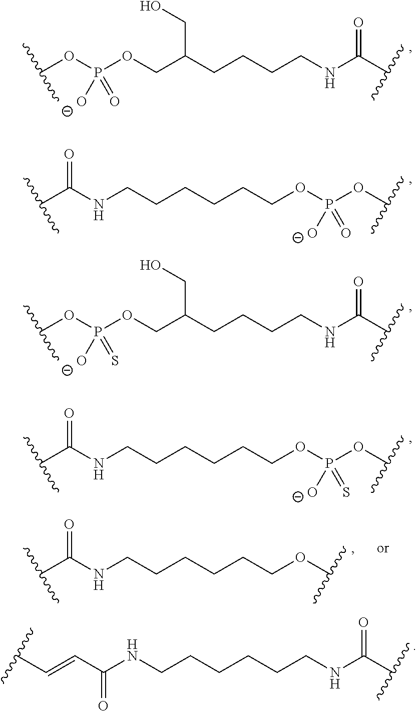 [SMILES: *OP(=O)([O-])OCC(CO)CCCCNC(*)=O ; *OP(=O)([O-])OCCCCCCNC(*)=O ; *OP([O-])(=S)OCC(CO)CCCCNC(*)=O ; *OP([O-])(=S)OCCCCCCNC(*)=O ; *OCCCCCCNC(*)=O ; */C=C/C(=O)NCCCCCCNC(*)=O]

Embodiment 113. The compound of Embodiment 112, wherein an -L³-L⁴- is independently

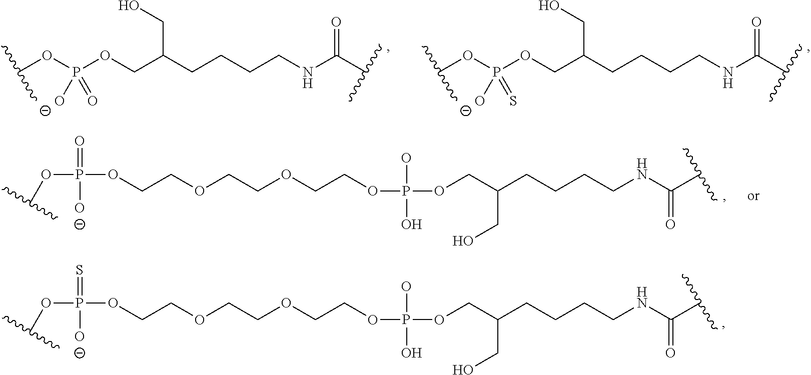

and is attached to a 3′ carbon of oligonucleotide.

Embodiment 114. The compound of Embodiment 112, wherein an -L³-L⁴- is independently

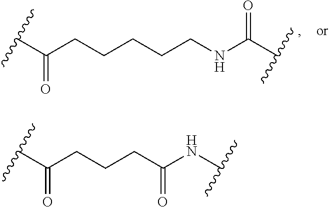 [SMILES: *C(=O)CCCCCNC(*)=O ; *NC(=O)CCCC(*)=O]

and is attached to a 3′ nitrogen of the oligonucleotide.

Embodiment 115. The compound of Embodiment 112, wherein an -L³-L⁴- is independently

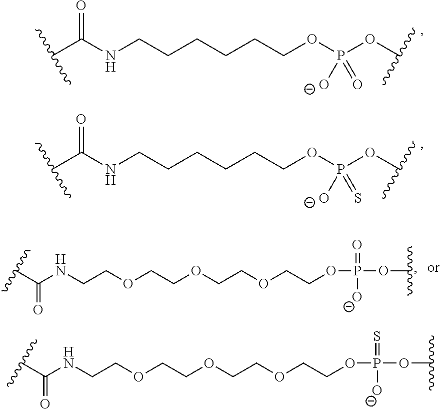 [SMILES: *OP(=O)([O-])OCCCCCCNC(*)=O ; *OP([O-])(=S)OCCCCCCNC(*)=O ; *OP(=O)([O-])OCCOCCOCCOCCNC(*)=O ; *OP([O-])(=S)OCCOCCOCCOCCNC(*)=O]

and is attached to a 5′ carbon of the oligonucleotide.

Embodiment 116. The compound of Embodiment 112, wherein an -L³-L⁴- is independently

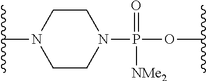 [SMILES: *OP(=O)(N(C)C)N1CCN(*)CC1]

and is attached to a 6′ carbon of the oligonucleotide.

Embodiment 117. The compound of Embodiment 112, wherein an -L³-L⁴- is independently

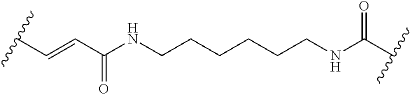 [SMILES: */C=C/C(=O)NCCCCCCNC(*)=O]

and is attached to a nucleobase of the oligonucleotide.

Embodiment 118. The compound of one of Embodiments 87 to 117, wherein R³ is independently hydrogen.

Embodiment 119. The compound of one of Embodiments 87 to 118, wherein L⁶ is independently —NHC(O)—, —C(O)NH—, substituted or unsubstituted alkylene, or substituted or unsubstituted heteroalkylene.

Embodiment 120. The compound of Embodiment 119, wherein L⁶ is independently —NHC(O)—.

Embodiment 121. The compound of Embodiment 119, wherein

L^{6A} is independently a bond or unsubstituted alkylene;

L^{6B} is independently a bond, —NHC(O)—, or unsubstituted arylene;

L^{6C} is independently a bond, unsubstituted alkylene, or unsubstituted arylene;

L^{6D} is independently a bond or unsubstituted alkylene; and

L^{6E} is independently a bond or —NHC(O)—.

Embodiment 122. The compound of Embodiment 119, wherein

L^{6A} is independently a bond or unsubstituted C₁-C₈ alkylene;

L^{6B} is independently a bond, —NHC(O)—, or unsubstituted phenylene;

L^{6C} is independently a bond, unsubstituted C₂-C₈ alkynylene, or unsubstituted phenylene;

L^{6D} is independently a bond or unsubstituted C₁-C₈ alkylene; and

L^{6E} is independently a bond or —NHC(O)—.

Embodiment 123. The compound of one of Embodiments 87 to 118, wherein L⁶ is independently a bond,

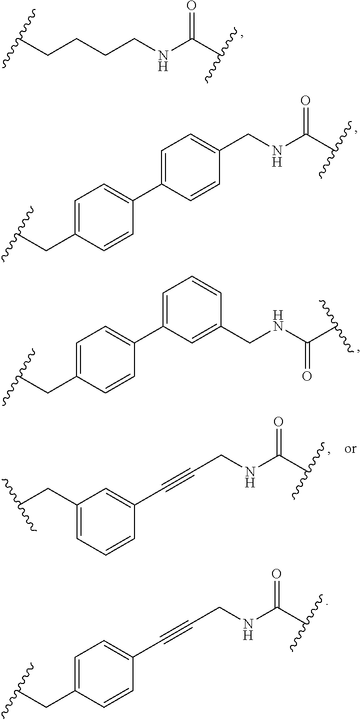 [SMILES: *CCCCNC(*)=O ; *Cc1ccc(-c2ccc(CNC(*)=O)cc2)cc1 ; *Cc1ccc(-c2cccc(CNC(*)=O)c2)cc1 ; *Cc1cccc(C#CCNC(*)=O)c1 ; *Cc1ccc(C#CCNC(*)=O)cc1]

Embodiment 124. The compound of one of Embodiments 87 to 123, wherein L⁵ is independently —NHC(O)—, —C(O)NH—, substituted or unsubstituted alkylene, or substituted or unsubstituted heteroalkylene.

Embodiment 125. The compound of one of Embodiments 87 to 123, wherein L⁵ is independently —NHC(O)—.

Embodiment 126. The compound of one of Embodiments 87 to 123, wherein

L^{5A} is independently a bond or unsubstituted alkylene;

L^{5B} is independently a bond, —NHC(O)—, or unsubstituted arylene;

L^{5C} is independently a bond, unsubstituted alkylene, or unsubstituted arylene;

L^{5D} is independently a bond or unsubstituted alkylene; and

L^{5E} is independently a bond or —NHC(O)—.

Embodiment 127. The compound of one of Embodiments 87 to 123, wherein

L^{5A} is independently a bond or unsubstituted C₁-C₈ alkylene;

L^{5B} is independently a bond, —NHC(O)—, or unsubstituted phenylene;

L^{5C} is independently a bond, unsubstituted C₂-C₈ alkynylene, or unsubstituted phenylene;

L^{5D} is independently a bond or unsubstituted C₁-C₈ alkylene; and

L^{5E} is independently a bond or —NHC(O)—.

Embodiment 128. The compound of one of Embodiments 87 to 123, wherein L⁵ is independently a bond,

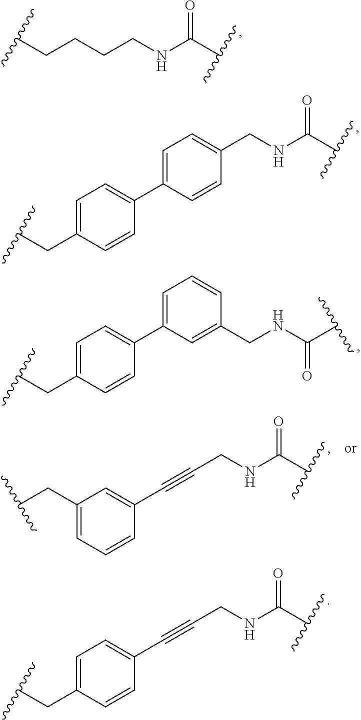 [SMILES: *CCCCNC(*)=O ; *Cc1ccc(-c2ccc(CNC(*)=O)cc2)cc1 ; *Cc1ccc(-c2cccc(CNC(*)=O)c2)cc1 ; *Cc1cccc(C#CCNC(*)=O)c1 ; *Cc1ccc(C#CCNC(*)=O)cc1]

Embodiment 129. The compound of one of Embodiments 87 to 123, wherein R¹ is unsubstituted C₁-C₁₇ alkyl.

Embodiment 130. The compound of one of Embodiments 87 to 123, wherein R¹ is unsubstituted C₁₁-C₁₇ alkyl.

Embodiment 131. The compound of one of Embodiments 87 to 123, wherein R¹ is unsubstituted C₁₃-C₁₇ alkyl.

Embodiment 132. The compound of one of Embodiments 87 to 123, wherein R¹ is unsubstituted C₁₄-C₁₅ alkyl.

Embodiment 133. The compound of one of Embodiments 87 to 123, wherein R¹ is unsubstituted unbranched C₁-C₁₇ alkyl.

Embodiment 134. The compound of one of Embodiments 87 to 123, wherein R¹ is unsubstituted unbranched C₁-C₁₇ alkyl.

Embodiment 135. The compound of one of Embodiments 87 to 123, wherein R¹ is unsubstituted unbranched C₁₃-C₁₇ alkyl.

Embodiment 136. The compound of one of Embodiments 87 to 123, wherein R¹ is unsubstituted unbranched C₁₄-C₁₅ alkyl.

Embodiment 137. The compound of one of Embodiments 87 to 123, wherein R¹ is unsubstituted unbranched saturated C₁-C₁₇ alkyl.

Embodiment 138. The compound of one of Embodiments 87 to 123, wherein R¹ is unsubstituted unbranched saturated C₁₁-C₁₇ alkyl.

Embodiment 139. The compound of one of Embodiments 87 to 123, wherein R¹ is unsubstituted unbranched saturated C₁₃-C₁₇ alkyl.

Embodiment 140. The compound of one of Embodiments 87 to 123, wherein R¹ is unsubstituted unbranched saturated C₁₄-C₁₅ alkyl.

Embodiment 141. The compound of one of Embodiments 87 to 140, wherein R² is unsubstituted C₁-C₁₇ alkyl.

Embodiment 142. The compound of one of Embodiments 87 to 140, wherein R² is unsubstituted C₁-C₁₇ alkyl.

Embodiment 143. The compound of one of Embodiments 87 to 140, wherein R² is unsubstituted C₁₃-C₁₇ alkyl.

Embodiment 144. The compound of one of Embodiments 87 to 140, wherein R² is unsubstituted C₁₄-C₁₅ alkyl.

Embodiment 145. The compound of one of Embodiments 87 to 140, wherein R² is unsubstituted unbranched C₁-C₁₇ alkyl.

Embodiment 146. The compound of one of Embodiments 87 to 140, wherein R² is unsubstituted unbranched C₁-C₁₇ alkyl.

Embodiment 147. The compound of one of Embodiments 87 to 140, wherein R² is unsubstituted unbranched C₁₃-C₁₇ alkyl.

Embodiment 148. The compound of one of Embodiments 87 to 140, wherein R² is unsubstituted unbranched C₁₄-C₁₅ alkyl.

Embodiment 149. The compound of one of Embodiments 87 to 140, wherein R² is unsubstituted unbranched saturated C₁-C₁₇ alkyl.

Embodiment 150. The compound of one of Embodiments 87 to 140, wherein R² is unsubstituted unbranched saturated C₁₁-C₁₇ alkyl.

Embodiment 151. The compound of one of Embodiments 87 to 140, wherein R² is unsubstituted unbranched saturated C₁₃-C₁₇ alkyl.

Embodiment 152. The compound of one of Embodiments 87 to 140, wherein R² is unsubstituted unbranched saturated C₁₄-C₁₅ alkyl.

Embodiment 153. The compound of one of Embodiments 12 to 152, wherein the oligonucleotide is a single-stranded oligonucleotide or a double-stranded oligonucleotide.

Embodiment 154. The compound of Embodiment 153, wherein the double-stranded oligonucleotide is a small interfering RNA, a short hairpin RNA or a microRNA mimic.

Embodiment 155. The compound of Embodiment 153, wherein the single-stranded oligonucleotide is a single-strand small interfering RNA, an RNaseH oligonucleotide, an anti-microRNA oligonucleotide, a steric blocking oligonucleotide, exon-skipping oligonucleotide, a CRISPR guide RNA, or an aptamer.

Embodiment 156. The compound of one of Embodiments 1 to 152, wherein the nucleic acid comprises one or more modified nucleotides.

Embodiment 157. The compound of any one of Embodiments 1 to 156, wherein the nucleic acid comprises one or more modified sugar moieties.

Embodiment 158. The compound of Embodiment 157, wherein the modified sugar moiety comprises a 2′ modification or an unlocked sugar modification.

Embodiment 159. The compound of Embodiment 158, wherein the 2′-modification is selected from 2′-fluoro modification, 2′-O-methyl modification, a 2′-O-methoxyethyl, and a bicyclic sugar modification.

Embodiment 160. The compound of Embodiment 159, wherein the bicyclic sugar modification is selected from a 4′-CH(CH₃)—O-2′ linkage, a 4′-(CH₂)₂—O-2′ linkage, a 4′-CH(CH₂—OMe)-O-2′ linkage, 4′-CH₂—N(CH₃)—O-2′ linkage, and 4′-CH₂—N(H)—O-2′ linkage.

Embodiment 161. The compound of Embodiment 157, wherein the modified sugar moiety is a morpholino moiety.

Embodiment 162. The compound of one of Embodiments 1 to 161, wherein the nucleic acid comprises one or more modified internucleotide linkages.

Embodiment 163. The compound of Embodiment 162, wherein the modified internucleotide linkage selected from a phosphorothioate linkage and a phosphorodiamidate linkage.

Embodiment 164. The compound of any one of Embodiments 156 to 163, wherein the nucleic acid is a small interfering RNA (siRNA) or a single-stranded small interfering RNA (ssRNAi) and the 5′ carbon at the 5′-terminus of the antisense strand comprises a hydroxyl group, a phosphate group, or modified phosphate group.

Embodiment 165. The nucleic acid compound of Embodiment 164, wherein the modified phosphate group is a 5′-(E)-vinylphosphonate.

Embodiment 166. The compound of Embodiment 154, wherein the oligonucleotide is a double-stranded oligonucleotide comprising an antisense strand hybridized to a sense strand and each of the antisense strand and sense strand is independently 15 to 30 nucleotides in length.

Embodiment 167. The compound of Embodiment 166, wherein each of the antisense strand and sense strand is 17 to 25 nucleotides in length.

Embodiment 168. The compound of Embodiment 166, wherein each of the antisense strand and sense strand is 19 to 23 nucleotides in length.

Embodiment 169. The compound of Embodiment 154, wherein the oligonucleotide is single-stranded and the oligonucleotide is 8 to 30 nucleotides in length.

Embodiment 170. The compound of Embodiment 169, wherein the oligonucleotide is 12 to 25 nucleotides in length.

Embodiment 171. The compound of Embodiment 169, wherein the oligonucleotide is 15 to 25 nucleotides in length.

Embodiment 172. The compound of Embodiment 169, wherein the oligonucleotide is 17 to 23 nucleotides in length.

Embodiment 173. The compound of one of Embodiments 1 to 172, wherein the compound is capable of binding a serum protein.

Embodiment 174. The compound of one of Embodiments 1 to 172, wherein the compound is capable of binding serum albumin.

Embodiment 175. The compound of one of Embodiments 1 to 172, wherein the compound has increased serum albumin binding compared to an identical compound lacking the one or more optionally different half-life extension motifs.

Embodiment 176. The compound of one of Embodiments 1 to 172, wherein the compound has an increased serum half-life compared to an identical compound lacking the one or more optionally different half-life extension motifs.

Embodiment 177. The compound of one of Embodiments 1 to 176, wherein the compound further comprises a ligand.

Embodiment 178. The compound of Embodiment 177, wherein the ligand comprises a peptide, an antibody, a carbohydrate, or an additional nucleic acid.

Embodiment 179. The compound of one of Embodiments 3 to 86, wherein the uptake motif comprises a peptide, an antibody, a carbohydrate, or an additional nucleic acid.

Embodiment 180. A method comprising contacting a cell with a compound of one of Embodiments 1 to 179.

Embodiment 181. The method of Embodiment 180, wherein the contacting occurs in vitro.

Embodiment 182. The method of Embodiment 180, wherein the contacting occurs ex vivo.

Embodiment 183. The method of Embodiment 180, wherein the contacting occurs in vivo.

Embodiment 184. A method comprising administering to a subject a compound of one of Embodiments 1 to 179.

Embodiment 185. The method of Embodiment 184, wherein the subject has a disease or disorder of the eye, liver, kidney, heart, adipose tissue, lung, muscle or spleen.

Embodiment 186. A compound of one of Embodiments 1 to 179, for use in therapy.

Embodiment 187. A compound of one of Embodiments 1 to 179, for use in the preparation of a medicament.

Embodiment 188. A method of introducing a nucleic acid into a cell within a subject, the method comprising administering to said subject the compound of one of Embodiments 1 to 179.

Embodiment 189. A cell comprising the compound of one of Embodiments 1 to 179.

Embodiment 190. A pharmaceutical composition comprising a pharmaceutically acceptable excipient and the compound of one of Embodiments 1 to 179.

### Examples

The following examples will further describe the present disclosure, and are used for the purposes of illustration only, and should not be considered as limiting.

The compounds disclosed herein may be synthesized by methods described below, or by modification of these methods. Ways of modifying the methodology include, among others, temperature, solvent, reagents, etc., known to those skilled in the art. In general, during any of the processes for preparation of the compounds disclosed herein, it may be necessary and/or desirable to protect sensitive or reactive groups on any of the molecules concerned. This may be achieved by means of conventional protecting groups, such as those described in Protective Groups in Organic Chemistry (ed. J. F. W. McOmie, Plenum Press, 1973); and P. G. M. Green, T. W. Wutts, Protecting Groups in Organic Synthesis (3rd ed.) Wiley, New York (1999), which are both hereby incorporated herein by reference in their entirety. The protecting groups may be removed at a convenient subsequent stage using methods known from the art. Synthetic chemistry transformations useful in synthesizing applicable compounds are known in the art and include, e.g., those described in R. Larock, Comprehensive Organic Transformations, VCH Publishers, 1989, or L. Paquette, ed., Encyclopedia of Reagents for Organic Synthesis, John Wiley and Sons, 1995, which are both hereby incorporated herein by reference in their entirety. The routes shown and described herein are illustrative only and are not intended, nor are they to be construed, to limit the scope of the claims in any manner whatsoever. Those skilled in the art will be able to recognize modifications of the disclosed syntheses and to devise alternate routes based on the disclosures herein; all such modifications and alternate routes are within the scope of the claims.

### Syntheses of Lipid Motifs

### Synthesis of DTx-01-01

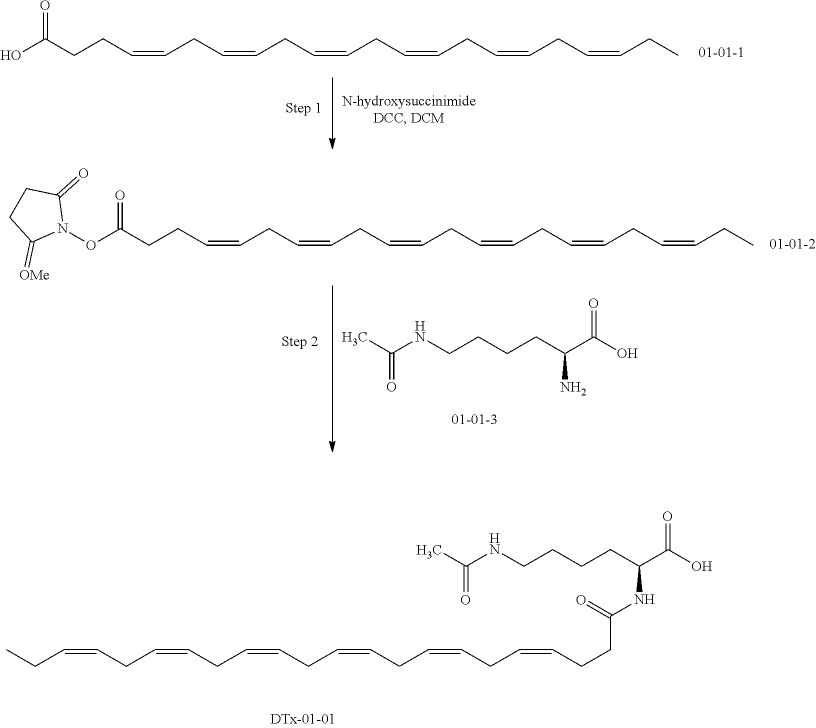

### Step 1: Synthesis of Intermediate 01-01-2

To a stirred solution of 01-01-1 (5.0 g, 0.015 mol) in DCM (500 mL) at RT was added DMAP (0.17 g, 0.0015 mol), DCC (4.86 g, 0.016 mol), followed by N-hydroxysuccinimide (1.92 g, 0.016 mol). The resulting mixture was stirred at RT. After 16 h, the reaction mixture was filtered through a sintered funnel. The filtrate was evaporated to yield crude 01-01-2 as a pale-yellow liquid (6.0 g, 92.5%), which was used in the next step without further purification.

### Step 2: Synthesis of Lipid Motif DTx-01-01

To a stirred solution of 01-01-3 (1.3 g, 0.006 mol) in DMF (20 mL) at RT was added slowly Et₃N (3 mL, 0.020 mol) and then 01-01-2 (2.93 g, 0.007 mol). The resulting mixture was stirred at RT. After 16 h, the reaction mixture was quenched with ice water dropwise and then extracted with EtOAc. The combined organic extract was washed with ice water, brine, dried over Na₂SO₄, and then evaporated to yield crude DTx-01-01, which was purified by column chromatography (3% MeOH in DCM) to afford lipid motif DTx-01-01 as a viscous, brown liquid (1.3 g, 51%). LCMS m/z (M+H)⁺: 499.4; ¹H-NMR (400 MHz, DMSO-d6): δ 0.92 (t, J=7.6 Hz, 3H), 1.24-1.66 (m, 10H), 1.82 (s, 3H), 2.02-2.33 (m, 7H), 2.73-2.98 (m, 9H), 3.94 (br s, 1H), 5.27-5.34 (m, 10H), 7.70 (br s, 1H), 7.78 (br s, 1H).

### Synthesis of DTx-01-03

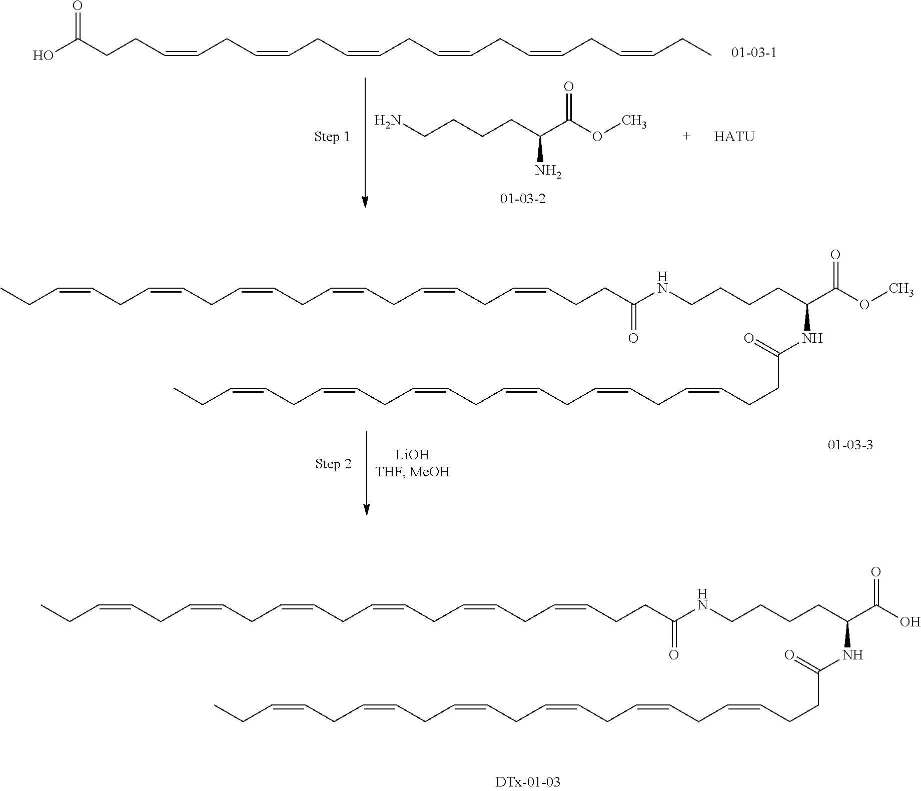

### Step 1: Synthesis of Intermediate 01-03-3

To a stirred solution of 01-03-1 (15 g, 0.045 mol) in DMF (300 mL) at RT was added slowly DIPEA (39.86 mL, 0.11 mol), HATU (17.1 g, 0.045 mol), and 01-03-2 (3.6 g, 0.022 mol). The resulting mixture was stirred at RT. After 16 h, the reaction mixture was quenched with ice water dropwise and extracted with DCM. The combined organic extract was washed with ice water, brine, dried over Na₂SO₄, and then evaporated to yield crude 01-03-3, which was purified by column chromatography (20% EtOAc in petroleum ether) to afford 01-03-3 as a viscous, pale brown liquid (11.2 g, 63.7%).

### Step 2: Synthesis of Lipid Motif DTx-01-03

To a stirred solution of 01-03-3 (10 g, 0.012 mol) in MeOH (100 mL) at 0° C. was added slowly LiOH (1.07 g, 0.025 mol) in water (50 mL). The resulting mixture was stirred at RT. After 4 h, ice water was added dropwise to the reaction mixture. The mixture was acidified with 1.5 M HCl and then extracted with DCM. The combined organic extract was washed with ice water, brine, dried over Na₂SO₄, and then evaporated to yield crude DTx-01-03, which was purified by column chromatography (3% MeOH in DCM) to afford lipid motif DTx-01-03 as a viscous, pale brown liquid (7.5 g, 77%). LCMS m/z (M+H)⁺: 767.5; ¹H-NMR (400 MHz, DMSO-d6): δ 0.954 (t, J=3.6 Hz, 6H), 1.23-1.66 (m, 8H), 1.99-2.33 (m, 12H), 2.69-2.82 (m, 22H), 4.13 (t, J=3.6 Hz, 1H), 5.25-5.36 (m, 22H), 7.76 (t, J=5.2 Hz, 1H), 8.03 (d, J=7.6 Hz, 1H), 12.5 (br s, 1H).

### Synthesis of Lipid Motif DTx-01-06

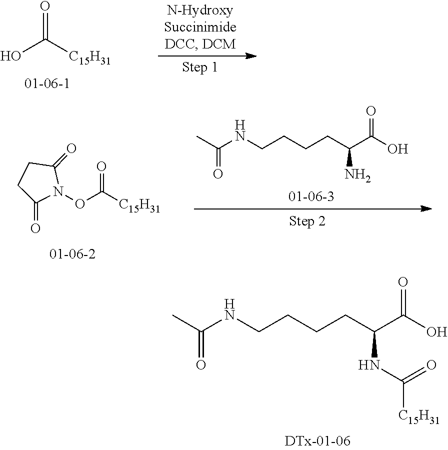

### Step 1: Synthesis of Intermediate 01-06-2

To a stirred solution of linear fatty acid 01-06-1 (5.0 g, 0.018 mol) in DCM (100 mL) at RT was added DMAP (0.208 g, 0.0018 mol), DCC (5.22 g, 0.018 mol), and then N-hydroxysuccinimide (2.07 g, 0.018 mol). The resulting mixture was stirred at RT. After 16 h, the reaction mixture was filtered through a sintered funnel. The filtrate was evaporated to yield crude 01-06-2 as an off-white solid (6.0 g, 88%), which was used in the next step without further purification.

### Step 2: Synthesis of Lipid Motif DTx-01-06

To a stirred solution of 01-06-3 (1.02 g, 0.054 mol) in DMF (40 mL) at RT was added slowly Et₃N (2.3 mL, 0.016 mol) and 01-06-2 (2 g, 0.047 mol). The resulting mixture was stirred at RT. After 16 h, the reaction mixture was quenched with ice water dropwise and then extracted with EtOAc. The combined organic extract was washed with chilled water, brine, dried over Na₂SO₄, and then evaporated to yield crude DTx-01-06, which was purified by column chromatography (3% MeOH in DCM) to afford lipid motif DTx-01-06 as an off-white solid (2.0 g, 88%). MS (ESI) m/z (M+H)⁺: 427.4; ¹H-NMR (400 MHz, DMSO-d6): δ 0.97 (t, J=7.2 Hz, 3H), 1.36-1.77 (m, 31H), 1.83 (s, 3H), 2.09 (t, J=6.4 Hz, 2H), 2.98 (d, J=6.0 Hz, 2H), 5.57 (d, J=8.0 Hz, 2H), 7.79 (br s, 1H), 7.97 (d, J=7.6 Hz, 1H).

### Synthesis of the Methyl Ester of Lipid Motif DTx-01-07 (DTx-01-07-OMe)

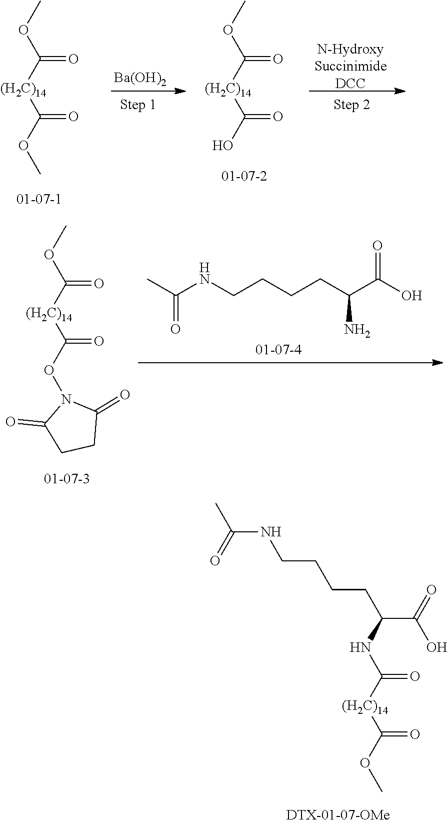

### Step 1: Synthesis of Intermediate 01-07-2

To a stirred solution of 01-07-1 (15 g, 0.063 mol) in MeOH (100 mL) at RT was added slowly Ba(OH)₂ (20 g, 0.063 mol). The resulting mixture was stirred at RT. After 16 h, the reaction mixture was quenched with ice water. The quenched reaction was acidified with 1.5 M HCl and then extracted with EtOAc. The combined organic extract was washed with water, brine, dried over Na₂SO₄, and then evaporated to yield crude 01-07-2. Purification by column chromatography (15% EtOAc in petroleum ether) afforded 01-07-2 as an off-white solid (15.2 g, 79.5%).

### Step 2: Synthesis of Intermediate 01-07-3

To a stirred solution of 01-07-2 (5.0 g, 0.016 mol) in DCM (500 mL) at RT was added DMAP (0.182 g, 0.0016 mol) and DCC (4.98 g, 0.016 mol), followed by N-hydroxy succinimide (2.1 g, 0.016 mol). The resulting mixture was stirred at RT. After 16 h, the reaction mixture was filtered through sintered funnel. The filtrate was evaporated to yield crude 01-07-3 as a pale-yellow liquid (5.0 g, 75%), which was used in the next step without further purification.

### Step 3: Synthesis of Lipid Motif DTx-01-07

To a stirred solution of 01-07-4 (0.94 g, 0.005 mol) in DMF (40 mL) at RT was added slowly Et₃N (2.12 mL, 0.015 mol) and then 01-07-3 (2.0 g, 0.005 mol). The resulting mixture was stirred at RT. After 16 h, the reaction mixture was quenched with ice water dropwise and then extracted with EtOAc. The combined organic extract was washed with ice water, brine, dried over Na₂SO₄, and then evaporated to yield crude DTx-01-07-OMe, which was purified by column chromatography (3% MeOH in DCM) to afford the methyl ester of lipid motif DTx-01-07 (i.e., DTx-01-07-OMe) as an off-white solid (2.0 g, 84%). LCMS m/z (M+H)⁺: 471.4; ¹H-NMR (400 MHz, DMSO-d6): δ 1.47-1.67 (m, 30H), 1.77 (s, 3H), 2.09 (t, J=7.2 Hz, 2H), 2.28 (d, J=7.2 Hz, 2H), 2.99 (q, J=6.4 Hz, 2H), 3.57 (s, 3H), 4.11 (t, J=4.8 Hz, 1H), 7.79 (br s, 1H), 7.97 (d, J=7.6 Hz, 1H).

### Synthesis of Lipid Motif DTx-01-08

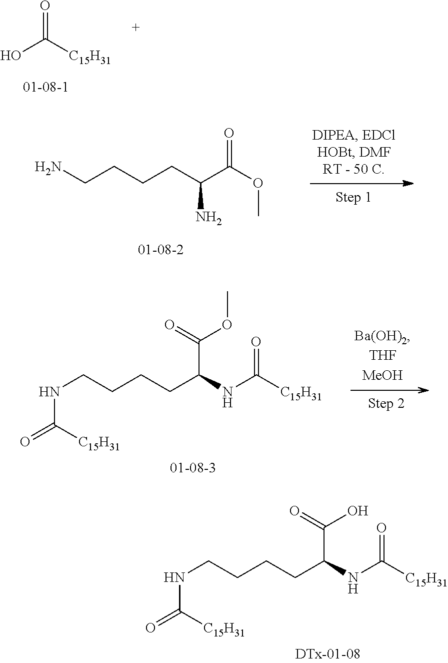

### Step 1: Synthesis of Compound 01-08-3

To a stirred solution of linear fatty acid 01-08-1 (25.58 g, 0.099 mol) in DMF (500 mL) at RT was added DIPEA (42.66 mL, 0.245 mol) and compound 01-08-2 (8.0 g, 0.049 mol), followed by EDCl (18.97 g, 0.099 mol) and HOBt (13.37 g, 0.099 mol). The resulting mixture was stirred at 50° C. After 16 h, the reaction mixture was quenched with ice water and extracted with DCM. The combined organic extract was washed with water, brine, dried over Na₂SO₄, and then evaporated to give crude 01-08-3, which was recrystallized (20% MTBE in petroleum ether) to afford 01-08-3 as an off-white solid (18 g, 56%).

### Step 2: Synthesis of Lipid Motif DTx-01-08

To a stirred solution of 01-08-3 (10 g, 0.0156 mol) in MeOH and THF (1:1; 200 mL) at RT was added slowly Ba(OH)₂ (9.92 g, 0.031 mol, dissolved in MeOH). The resulting mixture was stirred at RT. After 6 h, the reaction mixture was quenched with ice water dropwise, and then acidified with 1.5 M HCl. The mixture was filtered, and the precipitate was recrystallized (MTBE in petroleum ether) to afford lipid motif DTx-01-08 as an off-white solid (7.2 g, 74.2%). MS (ESI) m/z (M+H)⁺: 623.6; ¹H-NMR (400 MHz, CDCl₃): δ 0.868 (m, 6H), 1.25-1.69 (m, 58H), 2.03 (t, J=7.2 Hz, 2H), 2.11 (t, J=7.6 Hz, 2H), 2.99 (q, J=8.4 Hz, 2H), 4.15-4.20 (m, 1H), 7.42 (br s, 1H), 7.65 (d, J=7.6 Hz, 1H), 12.09 (br s, 1H).

### Synthesis of the Methyl Ester of Lipid Motif DTx-01-09 (DTx-01-09-OMe)

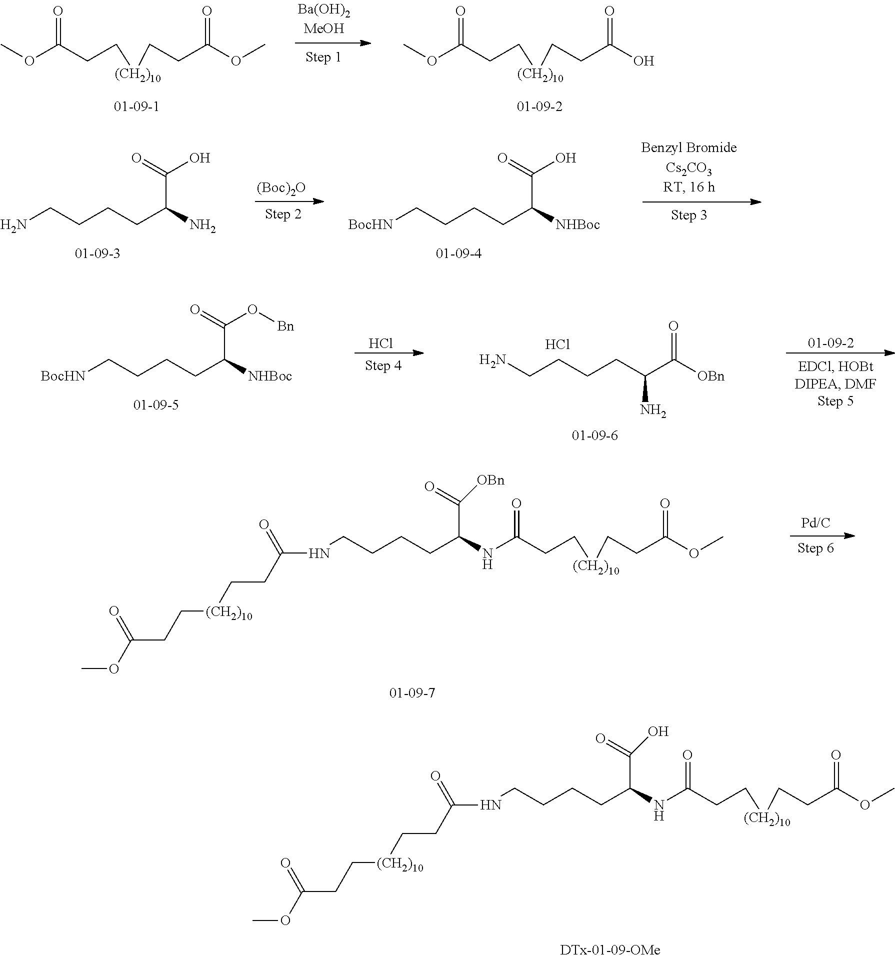

### Step 1: Synthesis of Intermediate 01-09-2

To a stirred solution of 01-09-1 (15 g, 0.063 mol) in MeOH (100 mL) at RT was added slowly Ba(OH)₂ (20 g, 0.063 mol). The resulting mixture was stirred at RT. After 16 h, the reaction mixture was quenched with ice water, acidified with 1.5 M HCl, and extracted with EtOAc. The combined organic extract was washed with water, brine, dried over Na₂SO₄, and then evaporated to yield crude 01-09-2, which was purified by column chromatography (15% EtOAc in petroleum ether) to afford product 01-09-2 as an off-white solid (15.2 g, 79.5%).

### Step 2: Synthesis of Intermediate 01-09-4

To a stirred solution of 01-09-3 (15 g, 0.102 mol) in 1,4-dioxane (100 mL) and water (50 mL) at RT was added slowly NaHCO₃ (18.98 g, 0.226 mol) and BOC anhydride (49.2 mL, 0.226 mol). The resulting mixture was stirred at RT. After 16 h, the reaction mixture was quenched with ice water dropwise and extracted with DCM. The combined organic extract was washed with ice water, brine, dried over Na₂SO₄, and then evaporated to yield crude 01-09-4, which was purified by column chromatography (30% EtOAc in petroleum ether) to afford 01-09-4 as viscous, pale yellow liquid (20 g, 56%).

### Step 3: Synthesis of Intermediate 01-09-5

To a stirred solution of 01-09-4 (15 g, 0.043 mol) in DMF (150 mL) at RT was added slowly Cs₂CO₃ (14 g, 0.043 mol) and benzyl bromide (5.6 mL, 0.047 mol). The resulting mixture was stirred at RT. After 16 h, the reaction mixture was quenched with ice water dropwise and extracted with EtOAc. The combined organic extract was washed with ice water, brine, dried over Na₂SO₄, and then evaporated to yield crude 01-09-5, which was purified by column chromatography (18% EtOAc in petroleum ether) to afford the 01-09-5 as a viscous, colorless liquid (15.2 g, 77%).

### Step 4: Synthesis of Intermediate 01-09-6

To a stirred solution of 01-09-5 (10 g, 0.022 mol) in 1,4-dioxane (50 mL) at RT was added slowly 4 M HCl in 1,4-dioxane (23 mL, 0.091 mol). The resulting mixture was stirred at RT. After 16 h, the reaction mixture was concentrated under reduced pressure. The residue was purified by trituration in diethyl ether, affording 01-09-6 as an off-white solid (15.2 g, 79.5%).

### Step 5: Synthesis of Intermediate 01-09-7

To a stirred solution of 01-09-6 (7.0 g, 0.025 mol) in DMF (100 mL) at RT was added slowly DIPEA (22.4 mL, 0.128 mol), 01-09-2 (15.05 g, 0.05 mol), EDCl (9.5 g, 0.05 mol), and HOBt (6.75 g, 0.05 mol). The resulting mixture was stirred at 50° C. After 16 h, the reaction mixture was quenched with ice water dropwise and extracted with DCM. The combined organic extract was washed with ice water, brine, dried over Na₂SO₄, and then evaporated to give crude 01-09-7. Recrystallization (MTBE in petroleum ether) yielded 01-09-7 as an off-white solid (10 g, 49.7%)

### Step 6: Synthesis of Lipid Motif DTx-01-09

To a stirred solution of 01-09-7 (10 g, 0.099 mol) in THF (100 mL) and EtOAc (100 mL) at RT was added 10% Pd/C (1.0 g). The resulting mixture was stirred at RT under 3 kg/Cm² hydrogen pressure. After 16 h, the mixture was filtered through celite, and the filtrate was evaporated to yield crude DTx-01-09-OMe. Recrystallization (20% MTBE in petroleum ether) afforded the methyl ester of lipid motif DTx-01-09 (i.e., DTx-01-09-OMe) as a pale yellow solid (5.3 g, 60%). LCMS m/z (M+H)⁺: 711.5; ¹H-NMR (400 MHz, CDCl₃): δ 1.23-1.52 (m, 55H), 2.01 (t, J=9.6 Hz, 2H), 2.08-2.11 (m, 2H), 2.28 (t, J=9.6 Hz, 4H), 2.99 (q, J=8.4 Hz, 2H), 3.57 (s, 6H), 4.11-4.12 (m, 1H), 7.72 (t, J=5.2 Hz, 1H), 7.96 (d, J=7.6 Hz, 1H).

### Synthesis of Lipid Motif DTx-01-11

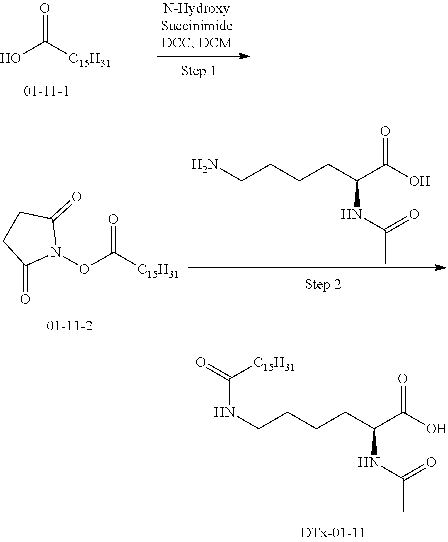

### Step 1: Synthesis of Intermediate 01-11-2

To a stirred solution of linear fatty acid 01-11-1 (5.0 g, 0.018 mol) in DCM (100 mL) at RT was added DMAP (0.208 g, 0.0018 mol) and DCC (5.22 g, 0.018 mol), followed by N-hydroxysuccinimide (2.07 g, 0.018 mol). The resulting mixture was stirred at RT. After 16 h, the reaction mixture was filtered through a sintered funnel. Evaporation of the filtrate yielded crude 01-11-2 as an off-white solid (6.0 g, 88%), which was used directly in the next step without further purification.

### Step 2: Synthesis of Lipid Motif DTx-01-11

To a stirred solution of 01-11-3 (2.05 g, 0.01 mol) in DMF (80 mL) at RT was added slowly Et₃N (4.6 mL, 0.032 mol) and 01-11-2 (4.0 g, 0.01 mol). The resulting mixture was stirred at RT. After 16 h, the reaction mixture was quenched with ice water dropwise and then extracted with EtOAc. The combined organic extract was washed with ice water, brine, dried over Na₂SO₄, and then evaporated to yield crude DTx-01-11, which was purified by column chromatography (3% MeOH in DCM) to afford lipid motif DTx-01-11 as an off-white solid (3.1 g, 66.5%). MS (ESI) m/z (M+H)⁺: 427.4; ¹H-NMR (400 MHz, DMSO-d6): δ 0.85 (t, J=6.8 Hz, 3H), 1.23-1.73 (m, 31H), 1.83 (s, 3H), 2.02 (t, J=7.2 Hz, 2H), 3.00 (q, J=6.0 Hz, 2H), 4.10 (dd, J=8.4, 4.4 Hz, 2H), 7.74 (d, J=5.2 Hz, 1H), 8.07 (br s, 1H), 12.45 (br s, 1H).

### Synthesis of the Methyl Ester of Lipid Motif DTx-01-12 (DTx-01-12-OMe)

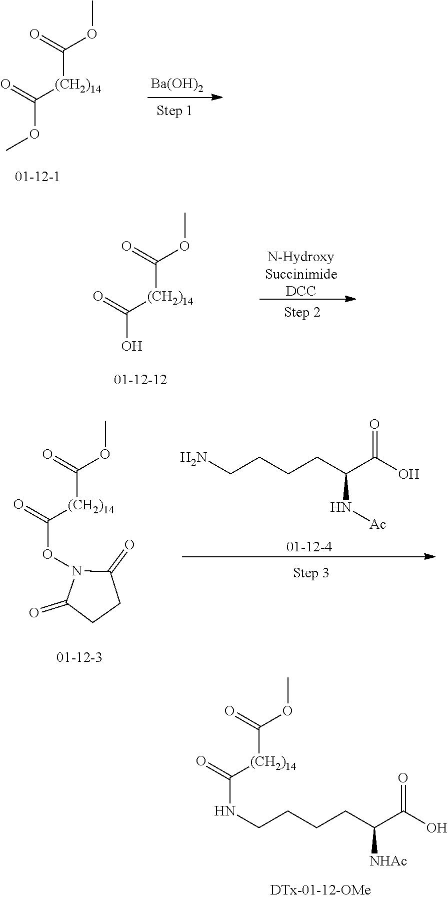

### Step 1: Synthesis of Intermediate 01-12-2

To a stirred solution of 01-12-1 (15 g, 0.063 mol) in MeOH (100 mL) at RT was added slowly Ba(OH)₂ (20 g, 0.063 mol). The resulting mixture was stirred at RT. After 16 h, the reaction mixture was quenched with ice water, acidified with 1.5 M HCl, and extracted with EtOAc. The combined organic extract was washed with water, brine, dried over Na₂SO₄, and then evaporated to yield crude 01-12-2. Purification by column chromatography (15% EtOAc in petroleum ether) afforded 01-12-2 as an off-white solid (15.2 g, 79.5%).

### Step 2: Synthesis of Intermediate 01-12-3

To a stirred solution of 01-12-2 (5.0 g, 0.016 mol) in DCM (500 mL) at RT was added DMAP (0.182 g, 0.0016 mol) and DCC (4.98 g, 0.016 mol), followed by N-hydroxysuccinimide (2.1 g, 0.016 mol). The resulting mixture was stirred at RT. After 16 h, the reaction mixture was filtered through a sintered funnel. The filtrate was evaporated to yield crude 01-12-3 as a pale yellow liquid (5.0 g, 75%), which was used directly in the next step without further purification.

### Step 3: Synthesis of Lipid Motif DTx-01-12

To a stirred solution of 01-12-4 (0.94 g, 0.005 mol) in DMF (40 mL) at RT was added slowly Et₃N (2.12 mL, 0.015 mol), 01-12-3 (2.0 g, 0.05 mol). The resulting mixture was stirred at RT. After 16 h, the reaction mixture was quenched with ice water dropwise and extracted with EtOAc. The combined organic extract was washed with ice water, brine, dried over Na₂SO₄, and then evaporated to yield crude DTx-01-12-OMe. Purification by column chromatography (3% MeOH in DCM) afforded the methyl ester of lipid motif DTx-01-12 (i.e., DTx-01-12-OMe) as an off-white solid (1.5 g, 63.2%). LCMS m/z (M+H)⁺: 471.4; ¹H-NMR (400 MHz, DMSO-d6): δ 1.22-1.66 (m, 30H), 1.83 (s, 3H), 2.01 (t, J=7.6 Hz, 2H), 2.27 (d, J=7.2 Hz, 2H), 2.99 (q, J=6.4 Hz, 2H), 3.57 (s, 3H), 4.10 (t, J=4.8 Hz, 1H), 7.72 (t, J=5.2 Hz, 1H), 8.06 (d, J=8.0 Hz, 1H), 12.47 (br s, 1H).

### Synthesis of Lipid Motif DTx-01-13

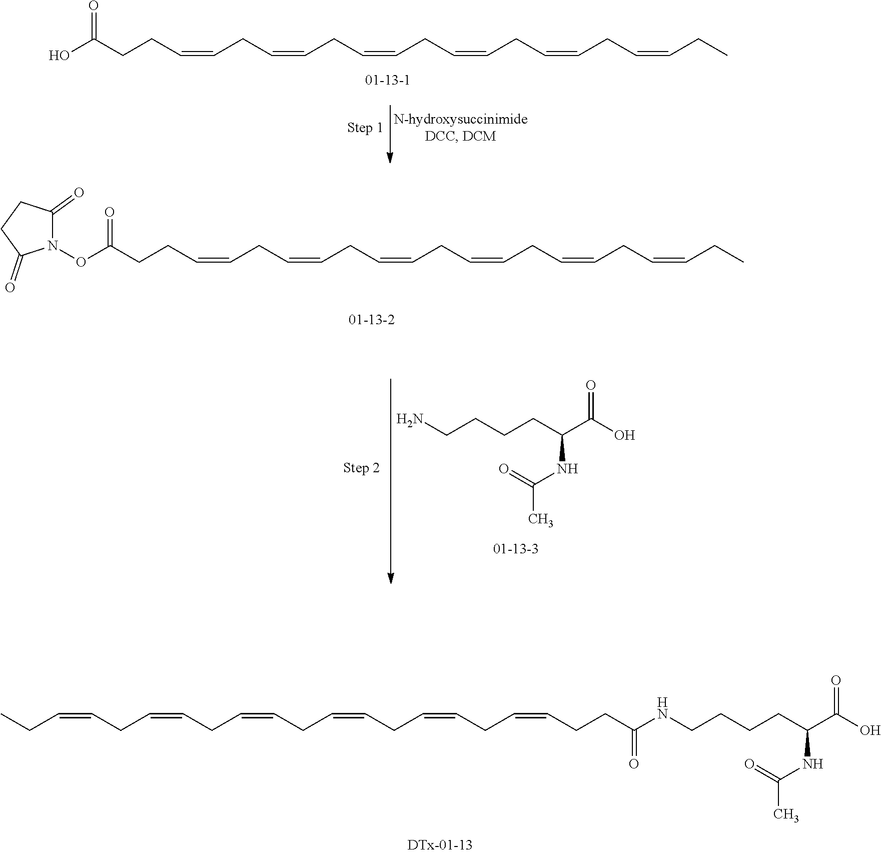

### Step 1: Synthesis of Intermediate 01-13-2

To a stirred solution of 01-13-1 (5.0 g, 0.015 mol) in DCM (500 mL) at RT was added DMAP (0.17 g, 0.0015 mol) and DCC (4.86 g, 0.016 mol), followed by N-hydroxysuccinimide (1.92 g, 0.016 mol). The resulting mixture was stirred at RT. After 16 h, the reaction mixture was filtered through a sintered funnel, and the filtrate was evaporated to yield crude 01-13-2 as a pale yellow liquid (6.0 g, 92.5%). The crude intermediate was used directly in the next step without further purification.

### Step 2: Synthesis of Lipid Motif DTx-01-13

To a stirred solution of 01-13-3 (1.3 g, 0.006 mol) in DMF (20 mL) at RT was added slowly Et₃N (3 mL, 0.020 mol) and 01-13-2 (2.93 g, 0.007 mol). The resulting mixture was stirred at RT. After 16 h, the reaction mixture was quenched with ice water dropwise and extracted with EtOAc. The combined organic extract was washed with ice water, brine, dried over Na₂SO₄, and then evaporated to yield crude DTx-01-13, which was purified by column chromatography (3% MeOH in DCM) to afford lipid motif DTx-01-13 as a viscous, brown liquid (2.1 g, 61%). LCMS m/z (M+H)⁺: 499.4; ¹H-NMR (400 MHz, DMSO-d6): δ 0.90 (t, J=7.2 Hz, 3H), 1.22-1.67 (m, 7H), 1.75 (s, 3H), 1.98-2.27 (m, 7H), 2.73-2.95 (m, 9H), 2.96 (dd, J=12.4, 6.4 Hz, 2H), 4.06-4.09 (m, 1H), 5.23-5.37 (m, 10H), 7.79 (br s, 1H), 7.91 (t, J=7.6 Hz, 1H).

### Synthesis of Lipid Motif DTx-01-30

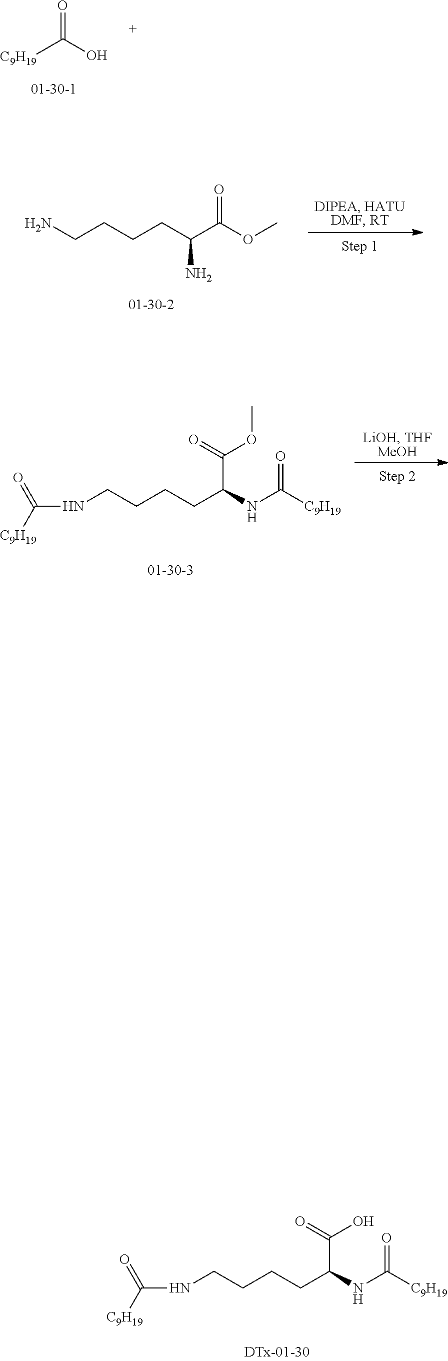

### Step 1: Synthesis of Intermediate 01-30-3

To a stirred solution of 01-30-2 (3 g, 0.01 mol) in DMF (50 mL) at RT was added slowly DIPEA (13.8 mL, 0.077 mol), linear fatty acid 01-30-1 (4.4 g, 0.0154 mol), and HATU (5.87 g, 0.0154 mol). The resulting mixture was stirred at RT. After 16 h, the reaction mixture was quenched with ice water. The precipitate was isolated by filtration, and then dried in vacuo to afford 01-30-3 as an off-white solid (3.2 g, 53.15%).

### Step 2: Synthesis of Lipid Motif DTx-01-30

To a stirred solution of 01-30-3 (3.2 g, 0.0068 mol) in MeOH (30 mL), THF (30 mL), and water (3 mL), was added LiOH.H₂O (0.86 g, 0.0251 mol). The resulting reaction mixture was stirred 16 h. Subsequently, the reaction mixture was concentrated under vacuum and then neutralized with 1.5 N HCl. The precipitate was isolated via filtration, washed with water, and dried under vacuum to yield crude DTx-01-30. Recrystallization (80% DCM in hexane) afforded lipid motif DTx-01-30 as an off-white solid (2.2 g, 73.3%). LCMS m/z (M+H)⁺: 455.5; ¹H-NMR (400 MHz, DMSO-d6): δ 0.88-0.92 (t, J=7.2 Hz, 6H), 1.17-1.55 (m, 33H), 1.64 (t, J=7.0 Hz, 1H), 2.00 (t, J=7.2 Hz, 2H), 2.06-2.10 (m, 2H), 2.97-2.99 (m, 2H), 4.11 (t, J=8.4 Hz, 1H), 7.71 (s, 1H), 7.96 (d, J=7.6 Hz, 1H), 12.47 (br s, 1H).

### Synthesis of Lipid Motif DTx-01-31

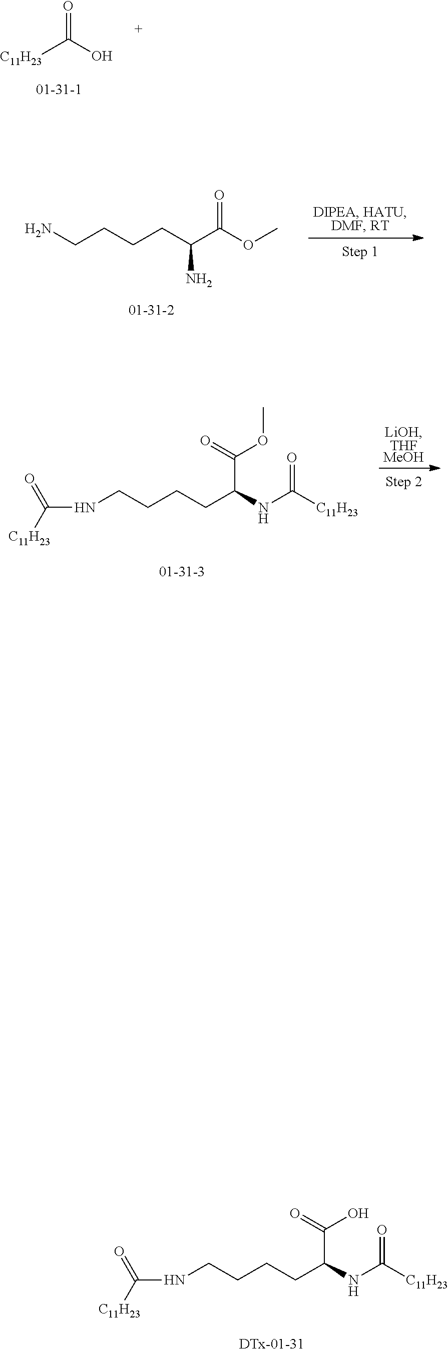

### Step 1: Synthesis of Intermediate 01-31-3

To a stirred solution of 01-31-2 (3 g, 0.0128 mol) in DMF (50 mL) at RT was added slowly DIPEA (13.8 mL, 0.077 mol), linear fatty acid 01-31-1 (3.1 g, 0.0154 mol), and HATU (5.87 g, 0.0154 mol). The resulting mixture was stirred at RT. After 16 h, the reaction mixture was quenched with ice water. Solids were isolated by filtration and dried in vacuo to afford 01-01-3 as an off-white solid (3.4 g 50.7%).

### Step 2: Synthesis of Lipid Motif DTx-01-31

To a stirred solution of 01-01-3 (3 g, 0.0057 mol) in MeOH (10 mL), THF (10 mL), and water (3 mL), was added LiOH.H₂O (0.8 g, 0.0019 mol). The reaction mixture was stirred 16 h. Subsequently, the reaction mixture was concentrated under vacuum and then neutralized with 1.5 N HCl. The precipitate was solid was isolated via filtration, washed with water, and dried under vacuum, yielding crude DTx-01-31. Recrystallization (80% DCM in hexane) afforded lipid motif DTx-01-31 as an off-white solid (2.3 g, 79.3%). LCMS m/z (M+H)⁺: 511.5; ¹H-NMR (400 MHz, DMSO-d6): δ 0.86-0.90 (t, J=7.2 Hz, 6H), 1.33-1.54 (m, 42H), 1.64 (t, J=7.9 Hz, 1H), 1.98-2.08 (m, 4H), 2.96 (t, J=6.3 Hz, 2H), 4.02-4.18 (m, 1H), 7.71-7.79 (m, 2H).

### Synthesis of Lipid Motif DTx-01-32

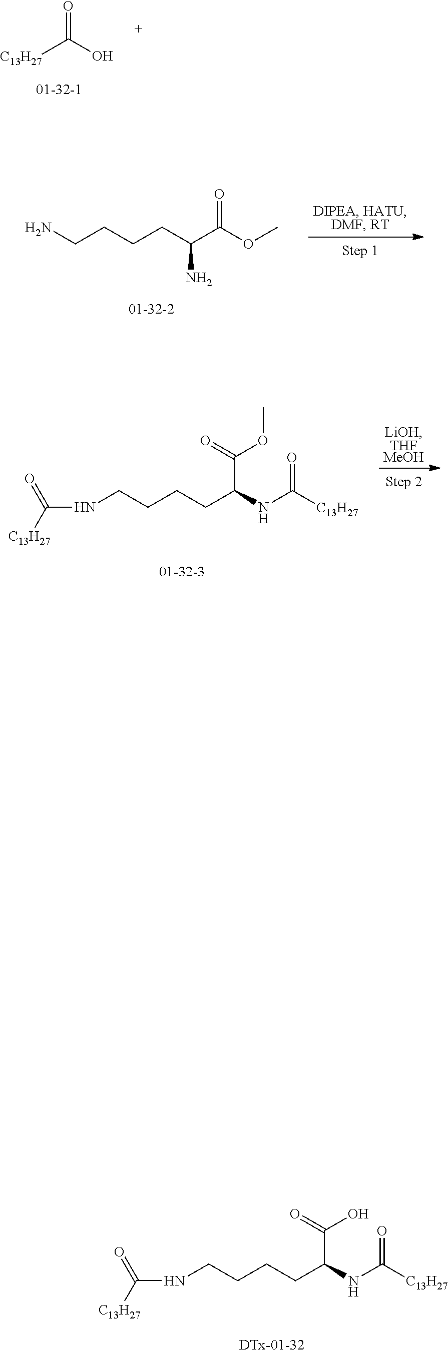

### Step 1: Synthesis of Intermediate 01-32-3

To a stirred solution of 01-32-2 (3 g, 0.01 mol) in DMF (50 mL) at RT was added slowly DIPEA (13.8 mL, 0.077 mol), linear fatty acid 01-32-1 (4.4 g, 0.0154 mol), and HATU (5.87 g, 0.0154 mol). The resulting mixture was stirred at 60° C. After 16 h, the reaction mixture was quenched with ice water, the solids isolated by filtration, and the solids dried under vacuum to afford 01-32-3 as an off-white solid (3.5 g, 53.2%).

### Step 2: Synthesis of Lipid Motif DTx-01-32

To a stirred solution of 01-32-3 (3.5 g, 0.0051 mol) in MeOH (10 mL), THF (10 mL), and water (3 mL), was added LiOH.H₂O (0.8 g, 0.0154). The reaction mixture was stirred 16 h. Subsequently, the reaction mixture was concentrated under vacuum and neutralized with 1.5 N HCl. The solids were isolated by filtration, washed with water, and dried under vacuum, affording crude DTx-01-32. Recrystallization (80% DCM in hexane) yielded lipid motif DTx-01-32 as an off-white solid (2.3 g, 79.3%). LCMS m/z (M+H)⁺: 567.2; ¹H-NMR (400 MHz, TFA-d): δ 0.87-0.98 (m, 6H), 1.20-1.58 (m, 41H), 1.74-1.92 (m, 8H), 2.18-2.21 (m, 2H), 2.73 (t, J=7.6 Hz, 2H), 3.05 (t, J=7.6 Hz, 2H), 3.60 (t, J=7.8 Hz, 2H).

### Synthesis of Lipid Motif DTx-01-33

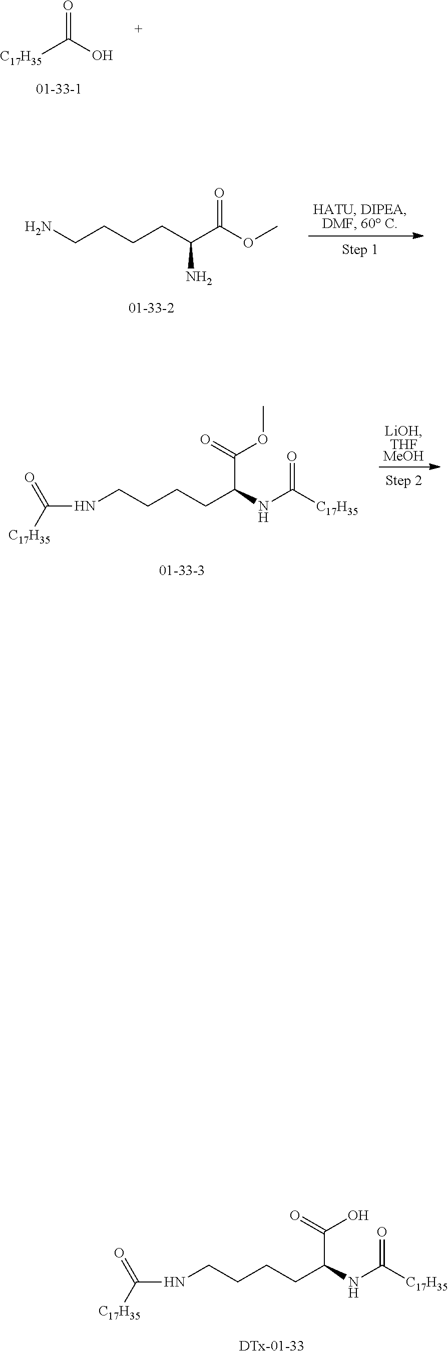

### Step 1: Synthesis of Intermediate 01-33-3

To a stirred solution of 01-33-2 (5 g, 0.0312 mol) in DMF (100 mL) at RT was added slowly DIPEA (32 mL, 0.1872 mol), linear fatty acid 01-33-1 (26.6 g, 0.0936 mol), and HATU (41.5 g, 0.1092 mol) slowly at RT. After 16 h, the reaction mixture was quenched with ice water. Crude 01-33-3 was isolated by filtration from the reaction mixture and dried in vacuo. Purification by trituration with THF afforded 01-33-3 as an off-white solid (8.5 g, 39.5%).

### Step 2: Synthesis of Lipid Motif DTx-01-33

To a stirred solution of 01-33-3 (5 g, 0.0072 mol) in MeOH (75 mL), THF (75 mL), and water (3 mL), was added LiOH.H₂O (0.60 g, 0.0144 mol). The reaction mixture was stirred 16 h. Subsequently, the reaction mixture was concentrated under vacuum and neutralized with 1.5 N HCl. The solids were filtered, washed with water, and dried under vacuum, affording crude DTx-01-33. Recrystallization (IPA) yielded lipid motif DTx-01-33 as an off-white solid (2.3 g, 47%). LCMS m/z (M+H)⁺: 680; ¹H-NMR (400 MHz, TFA-d): δ 1.10-1.18 (m, 6H), 1.62-1.80 (m, 57H), 2.06-2.20 (m, 8H), 2.49-2.50 (m, 2H), 2.96-3.01 (m, 2H), 3.32-3.35 (m, 2H), 3.87-3.98 (m, 2H).

### Synthesis of Lipid Motif DTx-01-34

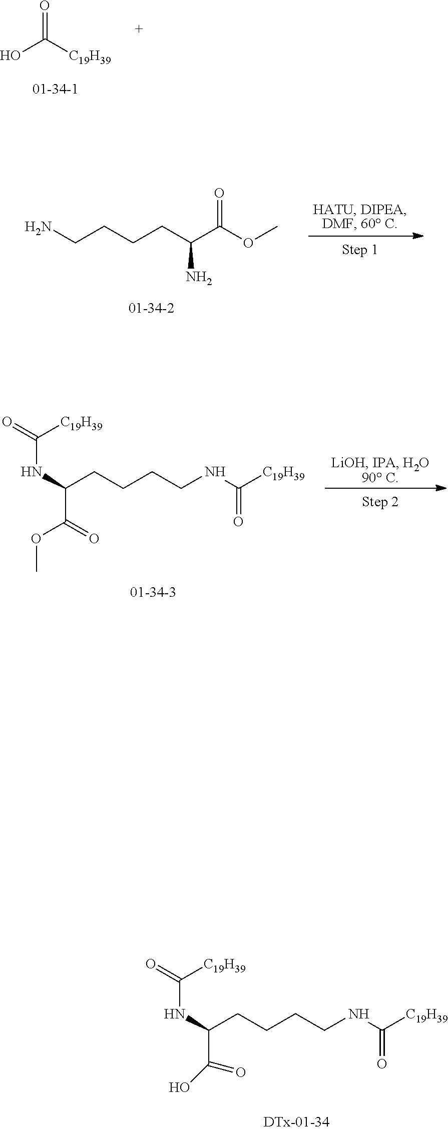

### Step 1: Synthesis of Intermediate 01-34-3

To a stirred solution of 01-34-2 (5 g, 0.0312 mol) in DMF (100 mL) at RT was added slowly DIPEA (32 mL, 0.1872 mol), linear fatty acid 01-34-1 (29.2 g, 0.0936 mol), and HATU (41.5 g, 0.1092 mol). The resulting mixture was stirred at 50° C. After 16 h, the reaction mixture was quenched with ice water, the solids isolated by filtration, and then the solids dried under vacuum. Purification of the solids by trituration with THF afforded 01-34-3 as an off-white solid (10 g, 43%).

### Step 2: Synthesis of Lipid Motif DTx-01-34

To a stirred solution of 01-34-3 (5 g, 0.0066 mol) in 9:1 IPA:water (150 mL) was added LiOH.H₂O (0.56 g, 0.0133 mol). The reaction mixture was stirred at 90° C. After 1 h, the reaction mixture was concentrated under vacuum and then neutralized with 1.5 N HCl. The precipitate was isolated via filtration, washed with water, and dried under vacuum. Recrystallization (IPA) of the precipitate afforded lipid motif DTx-01-34 as an off-white solid (3.2 g, 65%). LCMS m/z (M+H)⁺: 736.2; ¹H-NMR (400 MHz, TFA-d): δ 1.13-1.17 (m, 6H), 1.48-1.79 (m, 65H), 2.05-2.19 (m, 8H), 2.48-2.49 (m, 2H), 2.95-2.96 (m, 2H), 3.28-3.34 (m, 2H), 3.85-3.96 (m, 2H).

### Synthesis of Lipid Motif DTx-01-35

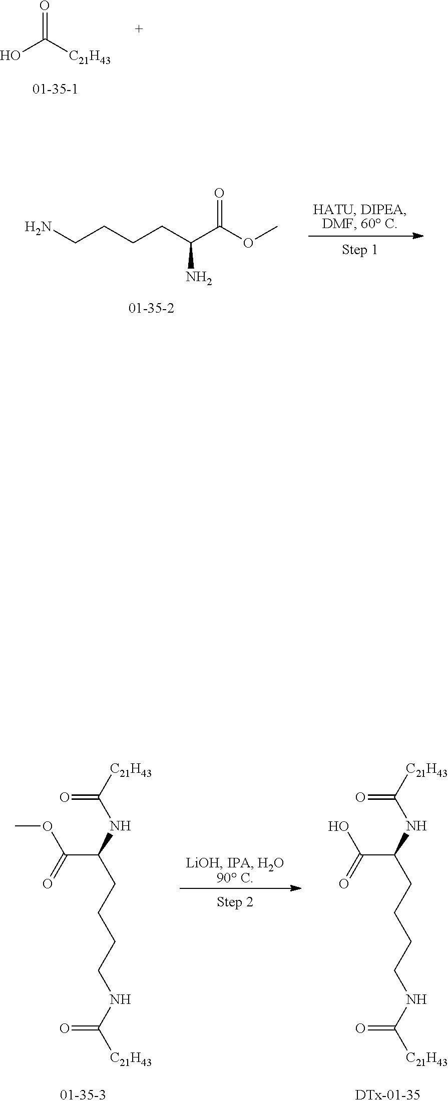

### Step 1: Synthesis of Intermediate 01-35-3

To a stirred solution of 01-35-2 (5 g, 0.0312 mol) in DMF (100 mL) at RT was added slowly DIPEA (32 mL, 0.1872 mol), linear fatty acid 01-35-1 (31.8 g, 0.0936 mol), and HATU (41.5 g, 0.1092 mol). The resulting mixture was stirred at 60° C. After 16 h, the reaction mixture was quenched with ice water, the solids isolated by filtration, and then the solids dried under vacuum. Purification of the solids by trituration with THF yielded 01-35-3 as an off-white solid (7 g, 28%).

### Step 2: Synthesis of Lipid Motif DTx-01-35

To a stirred solution of 01-35-3 (5 g, 0.0062 mol) in 9:1 IPA:water (150 mL) was added LiOH.H₂O (0.52 g, 0.0124 mol). The reaction mixture was stirred at 90° C. After 1 h, the reaction mixture was concentrated under vacuum and then neutralized with 1.5 N HCl. The solids were isolated by filtration, washed with water, and dried under vacuum, yielding crude DTx-01-35. Recrystallization in IPA afforded lipid motif DTx-01-35 as an off-white solid (3.1 g, 63%). LCMS m/z (M+H)⁺: 792.2; ¹H-NMR (400 MHz, TFA-d): δ 1.06-1.22 (m, 6H), 1.49-1.88 (m, 73H), 1.99-2.29 (m, 8H), 2.49-2.51 (m, 2H), 2.95-3.10 (m, 2H), 3.32-3.34 (m, 2H), 3.86-3.90 (m, 2H).

### Synthesis of Lipid Motif DTx-03-06

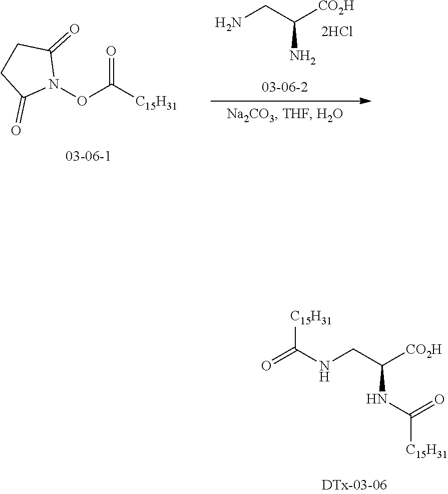

To a stirred solution of 03-06-2 (1.2 g, 0.0068 mol) in 65% aq. EtOH (40 mL) at RT was added slowly Et₃N (4.75 mL, 0.034 mol) and NHS-linear fatty acid 03-06-1 (6.0 g, 0.170 mol). The resulting mixture was stirred at 75° C. After 16 h, the reaction mixture was neutralized with 1.5 N HCl. The precipitate was isolated by filtration, washed with water, and dried. Purification of the precipitate by trituration with DCM afforded lipid motif DTx-03-06 as an off-white solid (2.3 g, 57%). LCMS m/z (M+H)⁺: 581.5; ¹H-NMR (400 MHz, TFA-d): δ 0.78-0.82 (m, 6H), 1.21-1.40 (m, 49H), 1.62-1.79 (m, 4H), 2.35-2.46 (m, 2H), 2.96-2.30 (m, 2H), 3.89-4.03 (m, 2H).

### Synthesis of Lipid Motif DTx-06-06

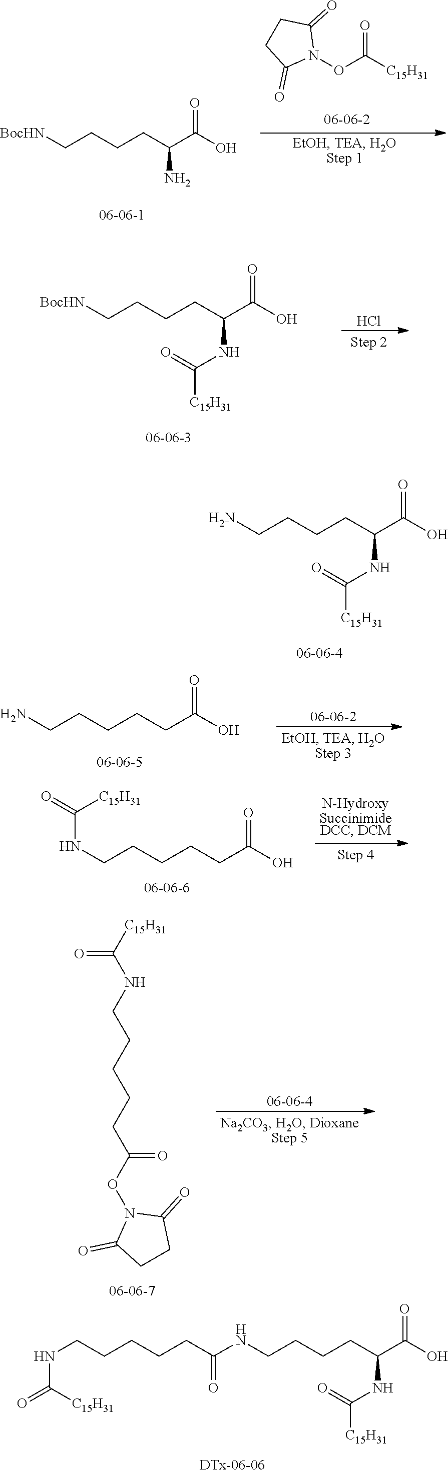

### Step 1: Synthesis of Intermediate 06-06-3

To a stirred solution of 06-06-1 (4.6 g, 0.0169 mol) in 65% aq. EtOH (60 mL) at RT was added slowly Et₃N (5.9 mL, 0.042 mol) and NHS-linear fatty acid 06-06-2 (6 g, 0.00186 mol). The resulting mixture was stirred at 75° C. After 16 h, the reaction mixture was neutralized with 1.5 N HCl. The precipitate was isolated by filtration, washed with water, and dried. Purification of the precipitate by column chromatography (3% MeOH in DCM) afforded 06-06-3 as an off-white solid (5.0 g, 62%).

### Step 2: Synthesis of Intermediate 06-06-4

To a stirred solution of 06-06-3 (7 g, 0.014 mol) in 1,4-dioxane (50 mL) at RT was added slowly 4 M HCl in 1,4-dioxane (50 mL). The resulting mixture was stirred at RT. After 16 h, the reaction mixture was concentrated under reduced pressure to yield crude 06-06-4, which was triturated with diethyl ether to afford 06-06-4 as an off-white solid (4.5 g, 81%).

### Step 3: Synthesis of Intermediate 06-06-6

To a stirred solution of 06-06-5 (5 g, 0.038 mol) in 65% aq. EtOH (40 mL) at RT was added slowly Et₃N (13.3 mL, 0.095 mol) and NHS-linear fatty acid 06-06-2 (13 g, 0.038 mol). The resulting mixture was stirred at 75° C. After 16 h, the reaction mixture was neutralized with 1.5 N HCl. The precipitate was isolated via filtration, washed with water, and dried, affording 06-06-6 as an off-white solid (4.2 g, 30%).

### Step 4: Synthesis of Intermediate 06-06-7

To a stirred solution of 06-06-6 (3.8 g, 0.010 mol) in DCM (80 mL) at RT was added DMAP (0.12 g, 0.001 mol) and DCC (2.1 g, 0.010 mol), followed by N-hydroxysuccinimide (1.17 g, 0.010 mol). The resulting mixture was stirred at RT 16 h. Subsequently, the reaction mixture was filtered through a sintered funnel, and then the filtrate evaporated, yielding crude 06-06-7 as an off-white solid (4.7 g, 100%), which was used in the next step without further purification.

### Step 5: Synthesis of Lipid Motif DTx-06-06

To a stirred solution of 06-06-4 (4 g, 0.009 mol) in 1 M Na₂CO₃ (50 mL) and 1,4-dioxane (100 mL) at RT was added slowly 06-06-7 (4.5 g, 0.096 mol). The resulting mixture was stirred at RT. After 16 h, the reaction mixture was neutralized with 1.5 N HCl. The precipitate was isolated by filtration, washed with water, and dried. Purification of the precipitate by trituration with MeOH afforded lipid motif DTx-06-06 as an off-white solid (2.3 g, 32%). LCMS m/z (M+H)⁺: 737.6; ¹H-NMR (400 MHz, TFA-d): δ 0.77-0.79 (m, 6H), 1.22-1.52 (m, 51H), 1.68-1.81 (m, 11H), 2.10-2.18 (m, 2H), 2.50-2.67 (m, 5H), 2.94-2.98 (m, 2H), 3.49-3.60 (m, 4H).

### Synthesis of Lipid Motif DTx-01-36

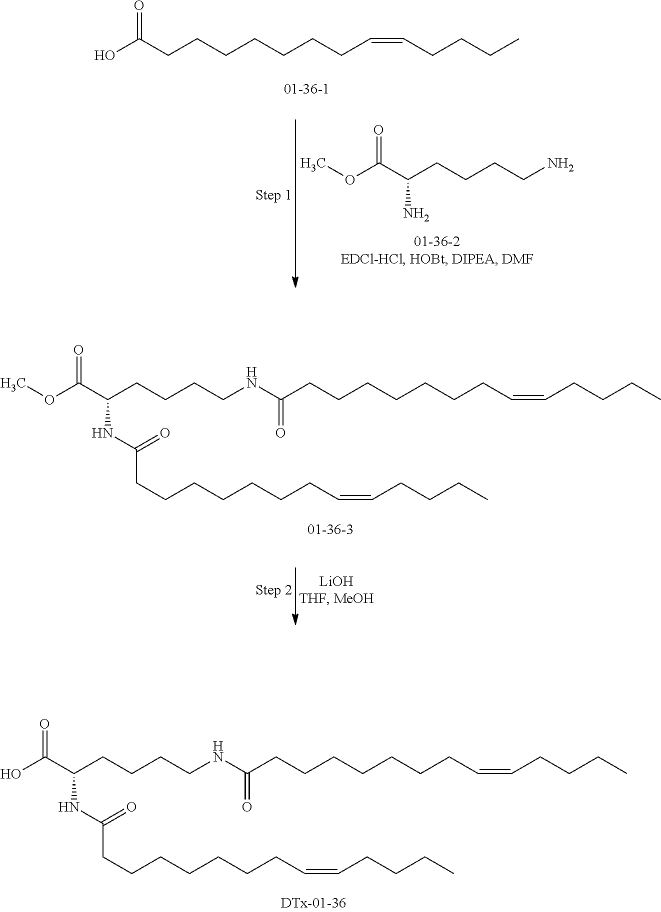

### Step 1:

To a stirred solution of 01-36-1 (0.73 g, 0.0032 mol) in DMF (6 mL) was added DIPEA (1.16 mL, 0.0064 mol), 01-36-2 (0.3 g, 0.0013 mol) followed by EDCl (0.543 g, 0.0028 mol), HOBt (0.382 g, 0.0028 mol) at RT. The resulting mixture was stirred at RT for 16 h. The reaction was monitored by LCMS. The reaction mixture was quenched with ice water and extracted with DCM. The combined organic extract was washed with water, brine, dried over Na₂SO₄, evaporated to give crude product which was further purified by column chromatography using 3% MeOH in DCM as eluent to get the product 01-36-3 as an off white solid. (0.54 g, 61%)

### Step 2:

To a stirred solution of compound 01-36-3 (0.5 g, 0.0009 mol) in MeOH, THF (10 mL; 1:1) and H₂O (0.25 mL) was added LiOH.H₂O (0.071 g, 0.0018 mol) and the reaction mixture was stirred at RT for 16 h. The reaction mixture was monitored by LCMS, the reaction mixture was concentrated under vacuum to give crude which was neutralized with 1.5 N HCl. Precipitated solid was extracted with DCM. The combined organic extract was washed with water, brine, dried over Na₂SO₄, evaporated to give crude product which was further purified by column chromatography using 3% MeOH in DCM as eluent to get the product DTx-01-36 as an off white solid. (0.35 g, 73%)

### Analytics of DTx-01-36

¹H-NMR- (400 MHz, DMSO-d6): δ 0.84 (t, J=6.8 Hz, 6H), 1.27-1.66 (m, 35H), 1.98-2.10 (m, 12H), 2.93-2.99 (m, 2H), 4.08-4.14 (m, 1H), 5.27-5.35 (m, 4H), 7.71 (t, J=5.2 Hz, 1H), 7.96 (d, J=7.6 Hz, 1H), 12.49 (bs, 1H). LCMS: 563.5 (M+1).

### Synthesis of Lipid Motif DTx-01-39

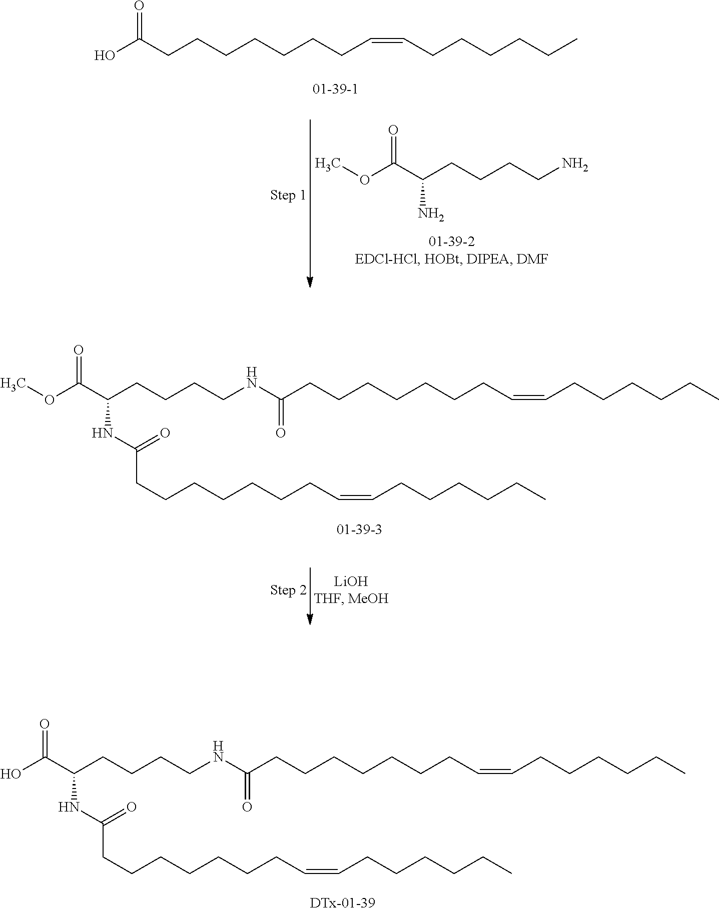

### Step 1:

To a stirred solution of compound 01-39-1 (2.04 g, 0.0080 mol) in DMF (20 mL) was added DIPEA (2.96 mL, 0.016 mol), compound 01-39-2 (0.75 g, 0.0032) followed by EDCl (1.35 g, 0.0070 mol), HOBt (0.95 g, 0.0070 mol) at RT. The resulting mixture was stirred at 50° C. for 16 h. The reaction was monitored by LCMS. The reaction mixture was quenched with ice water and extracted with DCM. The combined organic extract was washed with water, brine, dried over Na₂SO₄, evaporated to give crude product which was further purified by column chromatography using 3% MeOH in DCM as eluent to get the product 01-39-3 as an off white solid. (1.9 g, 79%)

### Step 2:

To a stirred solution of compound 01-39-3 (1.5 g, 0.0023 mol) in MeOH, THF (30 mL; 1:1) and H₂O (3 mL) was added LiOH.H₂O (0.194 g, 0.0046 mol) and the reaction mixture was stirred at RT for 16 h. The reaction mixture was monitored by LCMS, the reaction mixture was concentrated under vacuum to give crude which was neutralized with 1.5 N HCl. Precipitated solid was extracted with DCM. The combined organic extract was washed with water, brine, dried over Na₂SO₄, evaporated to give crude product which was further purified by column chromatography using 3% MeOH in DCM as eluent to get the product DTx-01-39 as yellow solid. (1.2 g, 82%)

### Analytics of DTx-01-39

¹H-NMR- (400 MHz, DMSO-d6): δ 0.83 (t, J=6.8 Hz, 6H), 1.23-1.78 (m, 42H), 1.96-2.08 (m, 12H), 2.98 (d, J=5.6 Hz, 2H), 4.08-4.10 (m, 1H), 5.28-5.31 (m, 4H), 7.71 (t, J=5.2 Hz, 1H), 7.95 (d, J=8.4 Hz, 1H), 12.43 (bs, 1H). LCMS: 619.5 (M+1).

### Synthesis of Lipid Motif DTx-01-43

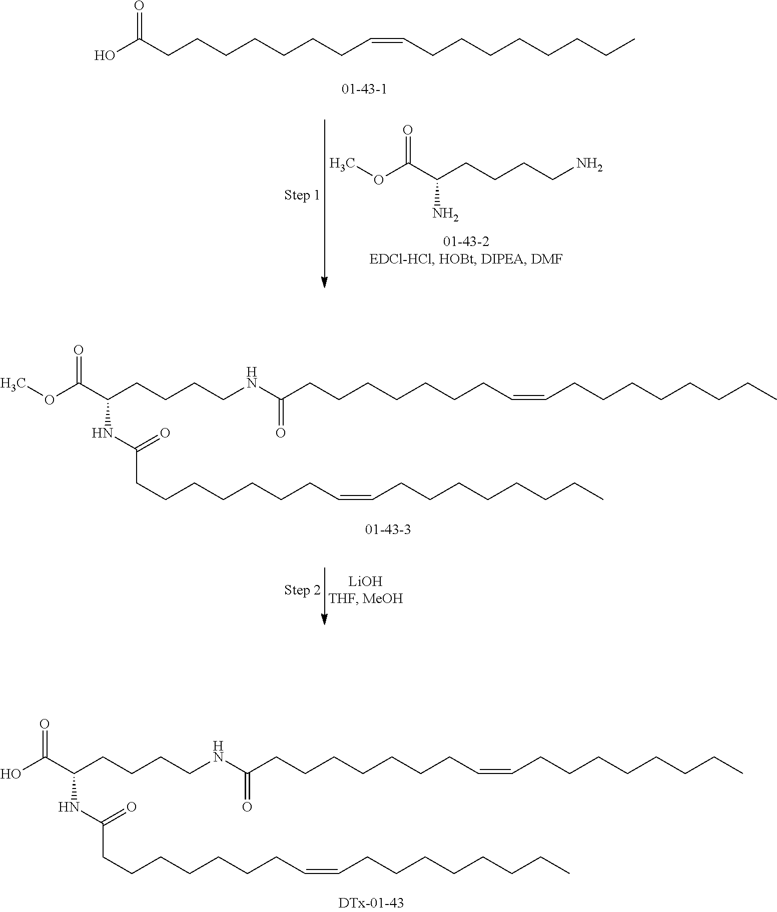

### Step 1:

To a stirred solution of compound 01-43-1 (3.5 g, 0.0107 mol) in DMF (50 mL) was added DIPEA (3.9 mL, 0.021 mol), compound 01-43-2 dihydrochloride (1 g, 0.0043 mol) followed by EDCl (1.8 g, 0.0094 mol), HOBt (1.2 g, 0.0094 mol) at RT. The resulting mixture was stirred at RT for 16 h. The reaction was monitored by LCMS. The reaction mixture was quenched with ice water and extracted with DCM. The combined organic extract was washed with water, brine, dried over Na₂SO₄, evaporated to give crude product which was further purified by column chromatography using 3% MeOH in DCM as eluent to get the product 01-43-3 as an off white solid. (2.6 g, 88.7%)

### Step 2:

To a stirred solution of compound 01-43-3 (2.5 g, 0.0036 mol) in MeOH, THF (40 mL; 1:1) and H₂O (2 mL) was added LiOH.H₂O (0.297 g, 0.0072 mol) and the reaction mixture was stirred at RT for 16 h. The reaction mixture was monitored by LCMS, the reaction mixture was concentrated under vacuum to give crude which was neutralized with 1.5 N HCl. Precipitated solid was extracted with DCM. The combined organic extract was washed with water, brine, dried over Na₂SO₄, evaporated to give crude product which was further purified by column chromatography using 3% MeOH in DCM as eluent to get the product DTx-01-43 as an off white solid. (2.1 g, 90.6%)

### Analytics of DTx-01-43

¹H-NMR- (400 MHz, DMSO-d6): δ 0.83 (t, J=6.8 Hz, 6H), 1.05-1.65 (m, 48H), 1.96-2.16 (m, 14H), 2.98-2.99 (m, 2H), 4.11-4.16 (m, 1H), 5.29-5.37 (m, 4H), 7.71 (bs, 1H), 7.92 (d, J=6.4 Hz, 1H). LCMS: 676.5 (M+1).

### Synthesis of Lipid Motif DTx-01-44

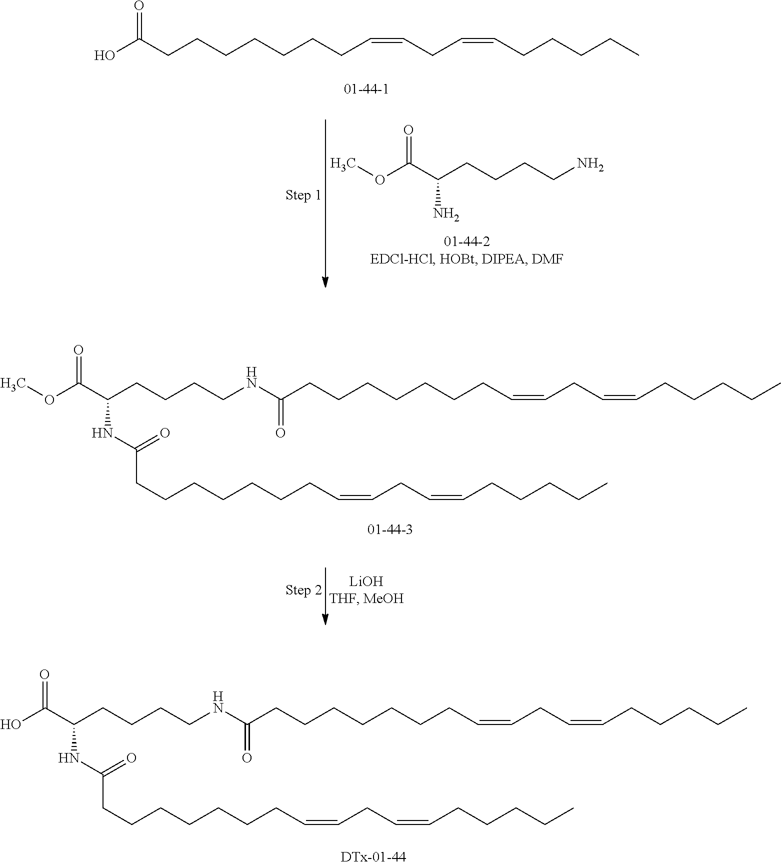

### Step 1:

To a stirred solution of compound 01-44-1 (5.1 g, 0.0018 mol) in DMF (50 mL) was added DIPEA (6.7 mL, 0.036 mol), compound 01-44-2 (1.7 g, 0.0072 mol) followed by EDCl (3.06 g, 0.016 mol), HOBt (2.16 g, 0.016 mol) at RT. The resulting mixture was stirred at RT for 16 h. The reaction was monitored by LCMS. The reaction mixture was quenched with ice water and extracted with DCM. The combined organic extract was washed with water, brine, dried over Na₂SO₄, evaporated to give crude product which was further purified by column chromatography using 3% MeOH in DCM as eluent to get the product 01-44-3 as an off white solid. (5 g, 85%)

### Step 2:

To a stirred solution of compound 01-44-3 (5 g, 0.0072 mol) in MeOH, THF (150 mL; 1:1) and H₂O (3 mL) was added LiOH.H₂O (0.60 g, 0.0144 mol) and the reaction mixture was stirred at RT for 16 h. The reaction mixture was monitored by LCMS, the reaction mixture was concentrated under vacuum to give crude which was neutralized with 1.5 N HCl. Precipitated solid was extracted with DCM. The combined organic extract was washed with water, brine, dried over Na₂SO₄, evaporated to give crude product which was further purified by column chromatography using 3% MeOH in DCM as eluent to get the product DTx-01-44 as pale yellow viscous liquid. (2.2 g, 45%)

### Analytics of DTx-01-44

¹H-NMR- (400 MHz, DMSO-d6): δ 0.86 (t, J=5.2 Hz, 6H), 1.25-1.70 (m, 38H), 2.01-2.18 (m, 12H), 2.73 (t, J=6.4 Hz, 4H), 2.98-3.00 (m, 2H), 4.12-4.24 (m, 1H), 5.29-5.36 (m, 8H), 7.72 (t, J=5.2 Hz, 1H), 7.95 (d, J=8.0 Hz, 1H), 12.45 (bs, 1H). LCMS: 672.6 (M+1).

### Synthesis of Lipid Motif DTx-01-45

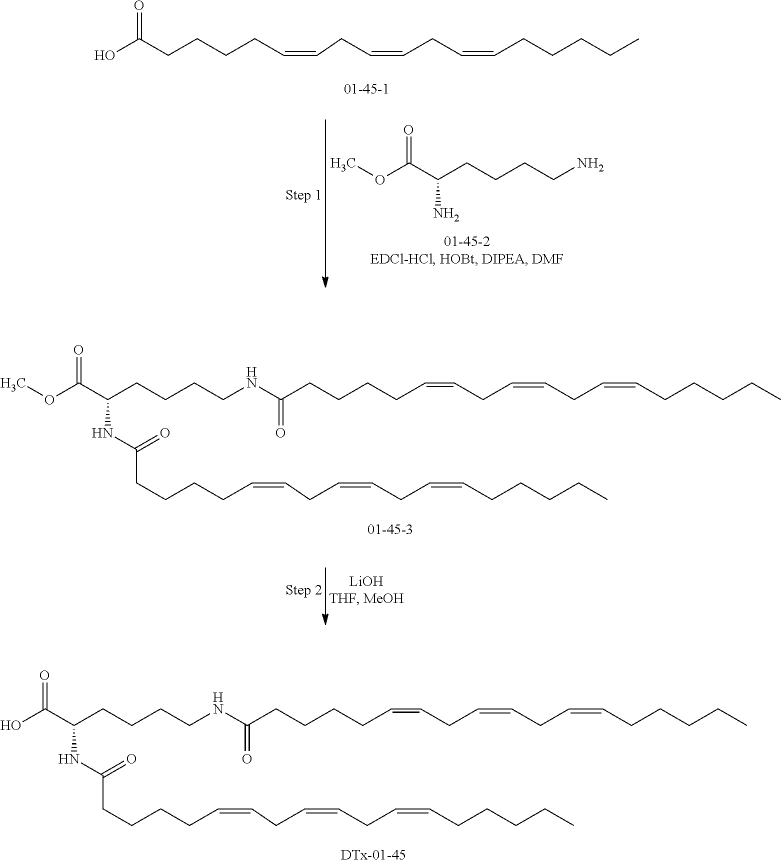

### Step 1:

To a stirred solution of compound 01-45-1 (0.656 g, 0.0023 mol) in DMF (5 mL) was added DIPEA (1.00 mL, 0.0053 mol), compound 04-45-2 dihydrochloride (0.25 g, 0.0011 mol) followed by EDCl (0.45 g, 0.0023 mol), HOBt (0.318 g, 0.0023 mol) at RT. The resulting mixture was stirred at RT for 16 h. The reaction was monitored by LCMS. The reaction mixture was quenched with ice water and extracted with DCM. The combined organic extract was washed with water, brine, dried over Na₂SO₄, evaporated to give crude product which was further purified by column chromatography using 3% MeOH in DCM as eluent to get the product 01-45-3 as an off white solid. (0.61 g, 83.56%)

### Step 2:

To a stirred solution of compound 04-45-3 (0.6 g, 0.0008 mol) in MeOH, THF (12 mL; 1:1) and H₂O (0.6 mL) was added LiOH.H₂O (0.074 g, 0.0018 mol) and the reaction mixture was stirred at RT for 16 h. The reaction mixture was monitored by LCMS, the reaction mixture was concentrated under vacuum to give crude which was neutralized with 1.5 N HCl. Precipitated solid was extracted with DCM. The combined organic extract was washed with water, brine, dried over Na₂SO₄, evaporated to give crude product which was further purified by column chromatography using 3% MeOH in DCM as eluent to get the product DTx-01-45 as an off white solid. (0.55 g, 94.8%)

### Analytics of DTx-01-45

¹H-NMR- (400 MHz, DMSO-d6): δ 0.86 (t, J=6.0 Hz, 6H), 1.27-1.50 (m, 26H), 2.01-2.10 (m, 12H), 2.77-2.80 (m, 8H), 2.96-2.98 (m, 2H), 3.98-4.01 (m, 1H), 5.32-5.37 (m, 12H), 7.61 (bs, 1H), 7.75 (bs, 1H). LCMS: 668.4 (M+1).

### Synthesis of DTx-01-46

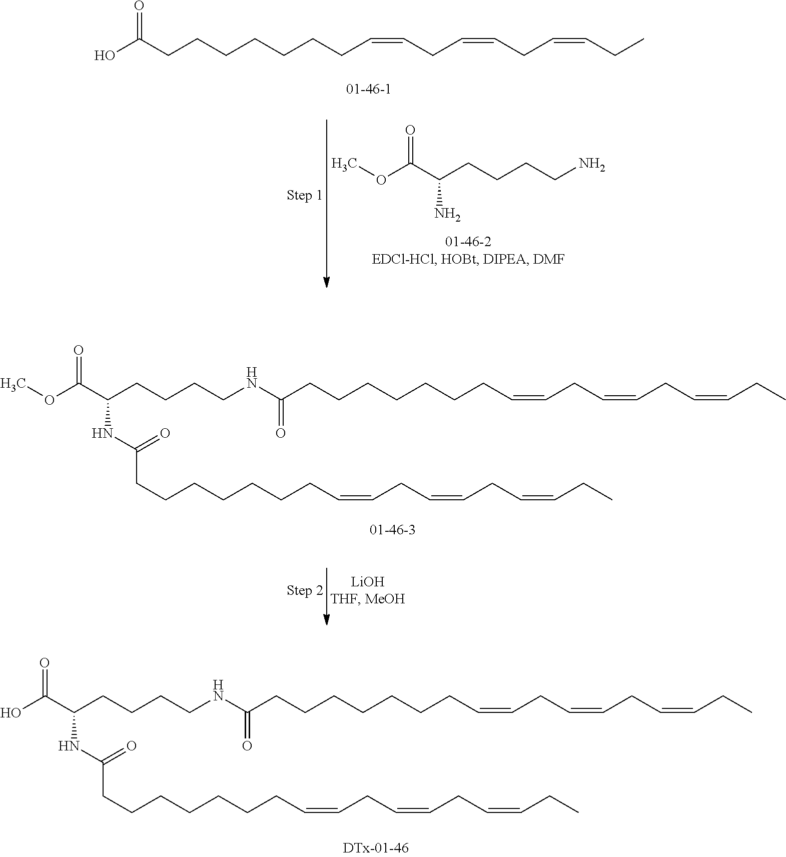

### Step 1:

To a stirred solution of compound 01-46-1 (2.00 g, 0.0071 mol) in DMF (20 mL) was added DIPEA (2.6 mL, 0.0143 mol), compound 01-46-2 (0.67 g, 0.0029 mol) followed by EDCl (1.20 g, 0.0063 mol), HOBt (0.085 g, 0.0063 mol) at RT. The resulting mixture was stirred at RT for 16 h. The reaction was monitored by LCMS. The reaction mixture was quenched with ice water and extracted with DCM. The combined organic extract was washed with water, brine, dried over Na₂SO₄, evaporated to give crude product which was further purified by column chromatography using 3% MeOH in DCM as eluent to get the product 01-46-3 as an off white solid. (1.8 g, 78%)

### Step 2:

To a stirred solution of compound 01-46-3 (2.4 g, 0.0035 mol) in MeOH, THF (75 mL; 1:1) and H₂O (2.5 mL) was added LiOH.H₂O (0.0288 g, 0.0070 mol) and the reaction mixture was stirred at RT for 16 h. The reaction mixture was monitored by LCMS, the reaction mixture was concentrated under vacuum to give crude which was neutralized with 1.5 N HCl. Precipitated solid was extracted with DCM. The combined organic extract was washed with water, brine, dried over Na₂SO₄, evaporated to give crude product which was further purified by column chromatography using 3% MeOH in DCM as eluent to get the product DTx-01-46 as pale yellow viscous liquid. (1.5 g, 64%)

### Analytics of DTx-01-46

¹H-NMR- (400 MHz, DMSO-d6): δ 0.91 (t, J=7.6 Hz, 6H), 1.24-1.68 (m, 31H), 2.01-2.10 (m, 10H), 2.78 (t, J=6.0 Hz, 4H), 2.88-2.99 (m, 3H), 5.27-5.36 (m, 1H), 5.29-5.36 (m, 12H), 7.71 (t, J=5.2 Hz, 1H), 7.96 (d, J=8.0 Hz, 1H). LCMS: 668.6 (M+1).

### Synthesis of DTx-08-01

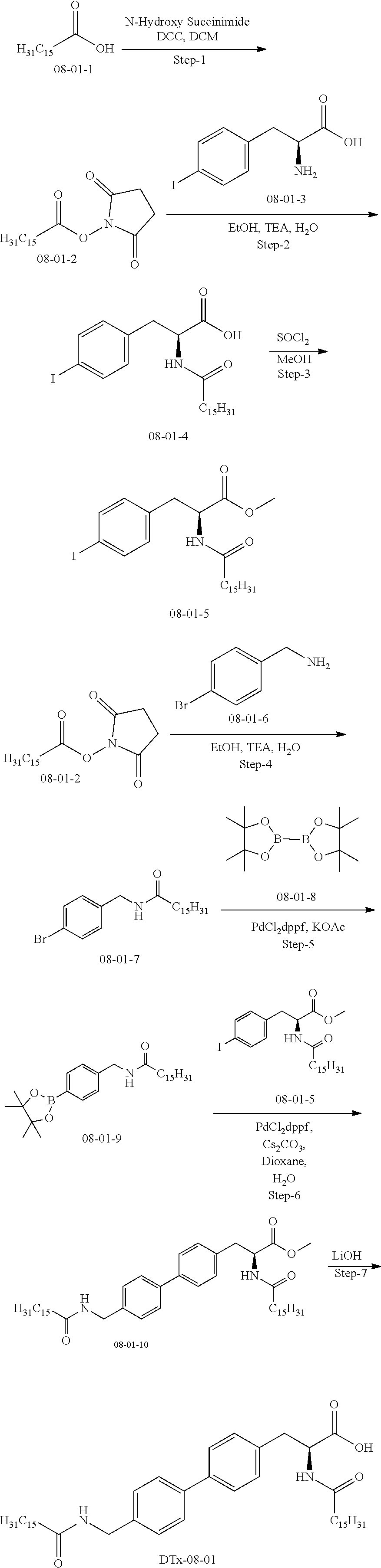

### Step 1:

To a stirred solution of compound 08-01-1 (10 g, 0.0389 mol) in DCM (200 mL) was added DMAP (0.47 g, 0.0038 mol), DCC (8.04 g, 0.0389 mol) followed by N-hydroxysuccinimide (4.48 g, 0.0389 mol) at RT. The resulting mixture was stirred at RT for 16 h. The reaction was monitored by LCMS. The reaction mixture was filtered through sintered funnel, the filtrate was evaporated to give crude product 08-01-02 as an off white solid which was directly proceeded for next step (10 g, 72%).

### Step 2:

To a stirred solution of compound 08-01-2 (10 g, 0.0283 mol) in 65% aq. ethanol (100 mL) was added Et₃N (11.8 mL, 0.0849 mol), compound 08-01-3 (10.6 g, 0.0368 mol) slowly at RT. The resulting mixture was stirred at 75° C. for 16 h. The reaction was monitored by LCMS. The reaction mixture was neutralized with 1.5 N HCl, precipitated solid was filtered, washed with water and dried to get the product 08-01-4 as an off white solid. (11 g, 73%)

### Step 3:

To a stirred solution of compound 08-01-4 (11 g, 0.0207 mol) in methanol (110 mL) was added thionyl chloride (44 mL) slowly at RT. The resulting mixture was stirred at RT for 16 h. The reaction was monitored by LCMS. The reaction mixture was concentrated under reduced pressure to get crude product which was triturated with diethyl ether to get pure compound of 08-01-5 as an off white solid (9 g, 80%).

### Step 4:

To a stirred solution of compound 08-01-2 (5 g, 0.0141 mol) in 65% aq. ethanol (50 mL) was added Et₃N (6 mL, 0.0424 mol), compound 08-01-6 (3.3 g, 0.0184 mol) slowly at RT. The resulting mixture was stirred at 75° C. for 16 h. The reaction was monitored by LCMS. The reaction mixture was neutralized with 1.5 N HCl, precipitated solid was filtered, washed with water and dried to get the product 08-01-7 as an off white solid. (5.1 g, 85%)

### Step 5:

To a stirred solution of compound 08-01-7 (5 g, 0.0117 mol) in dioxane (100 mL) was added 08-01-8 ((4,4,4′,4′,5,5,5′,5′-octamethyl-2,2′-bi(1,3,2-dioxaborolane) (4.4 g, 0.0176 mol)) and AcOK (3.4 g, 0.0353 mol). After degassing with nitrogen, Pd(dppf)Cl₂ (0.48 g, 0.0005 mol) was added to the reaction mixture. The resulting mixture was stirred at 90° C. for 12 h. The reaction mixture was monitored by LCMS, the reaction mixture was filtered through celite bed and concentrated under vacuum to give crude product which was further purified by column chromatography using 3% MeOH in DCM as eluent to get the product 01-08-9 as brown solid. (4.8 g, 86%)

### Step 6:

To a stirred solution of compound 01-08-5 (4.5 g, 0.0082 mol) in dioxane (90 mL) and water (9 mL) was added compound 01-08-9 (4.68 g, 0.0099 mol) and Cs₂CO₃ (8.1 g, 0.0248 mol). After degassing with nitrogen, Pd(dppf)Cl₂ (0.67 g, 0.0008 mol) was added to the reaction mixture. The resulting mixture was stirred at 90° C. for 3 h. The reaction mixture was monitored by LCMS, the reaction mixture was filtered through celite bed and concentrated under vacuum to give crude product which was further purified by column chromatography using 3% MeOH in DCM as eluent to get the product 01-08-10 as brown solid. (1 g, 14.2%)

### Step 7:

To a stirred solution of compound 01-08-10 (1 g, 0.0013 mol) in MeOH, THF (6.5 mL; 13 mL) and H₂O (6.5 mL) was added LiOH.H₂O (0.16 g, 0.0039 mol) and the reaction mixture was stirred at 50° C. for 3 h. The reaction mixture was monitored by LCMS, the reaction mixture was concentrated under vacuum. The resultant product was neutralized with 1.5 N HCl, the solid which was precipitated was filtered, washed with water and dried under vacuum to get the crude product. The crude product was triturated with MeOH to obtained pure DTx-08-01 as off white solid (0.5 g, 51%).

### Analytics of DTx-08-01

¹H-NMR- (400 MHz, TFA-d1): δ 0.78-0.79 (m, 6H), 1.08-1.49 (m, 48H), 1.49-1.50 (m, 2H), 1.72-1.83 (m, 2H), 2.69-2.71 (m, 2H), 5.77-2.82 (m, 2H), 3.41 (d, J=14.8 Hz, 1H), 3.53 (d, J=14.4 Hz, 1H), 4.66 (s, 2H), 5.16-5.18 (m, 1H), 7.23 (d, J=8.0 Hz, 2H), 7.33 (d, J=8.0 Hz, 2H), 7.58 (t, J=2.4 Hz, 4H). LCMS: 748.6 (M+1).

### Synthesis of DTx-09-01

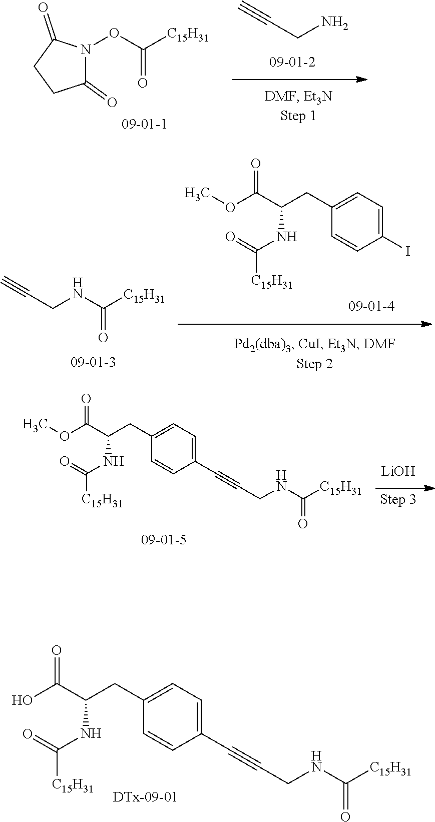

### Step 1:

To a stirred solution of compound 09-01-1 (10 g, 0.0283 mol) in DMF (100 mL) was added Et₃N (11.7 mL, 0.0849 mol), compound 09-01-2 (2.02 g, 0.0368 mol) slowly at RT. The resulting mixture was stirred at 50° C. for 16 h. The reaction was monitored by LCMS. The reaction mixture was neutralized with 1.5 N HCl, precipitated solid was filtered, washed with water and dried to get the product 09-01-3 as an off white solid. (4.5 g, 55%)

### Step 2:

To a stirred solution of compound 09-01-4 (5 g, 0.092 mol) in DMF (50 mL) was added compound 09-01-3 (3.5 g, 0.0119 mol), TEA (15 mL) and CuI (0.20 g, 0.0011 mol). After degassing with nitrogen, Pd₂(dba)₃ (0.67 g, 0.0007 mol) was added to the reaction mixture. The resulting mixture was stirred at 50° C. for 3 h. The reaction mixture was monitored by LCMS, the reaction mixture was filtered through celite bed and concentrated under vacuum to give crude product which was further purified by column chromatography using 25% EtOAc in Hexane as eluent to get the product 09-01-5 as off white solid. (1 g, 15.6%)

### Step 3:

To a stirred solution of compound 09-01-5 (1 g, 0.0014 mol) in MeOH, THF (6.5 mL; 13 mL) and H₂O (6.5 mL) was added LiOH.H₂O (0.17 g, 0.0042 mol) and the reaction mixture was stirred at 50° C. for 2 h. The reaction mixture was monitored by LCMS, the reaction mixture was concentrated under vacuum to give crude which was neutralized with 1.5 N HCl, precipitated solid was filtered, washed with water and dried under vacuum to get the crude product. The crude product was further purified by column chromatography using 3% MeOH in DCM as eluent to get the product DTx-09-01 as off pale brown solid (0.5 g, 51%).

### Analytics of DTx-09-01

¹H-NMR- (400 MHz, TFA-d1): δ 0.89-0.92 (m, 6H), 1.20-1.40 (m, 49H), 1.67-1.70 (m, 2H), 1.82-1.86 (m, 2H), 2.71-2.75 (m, 2H), 5.91-2.95 (m, 2H), 3.47 (d, J=14.8 Hz, 1H), 3.61 (d, J=14.8 Hz, 1H), 4.52 (s, 2H), 7.25 (d, J=8.0 Hz, 2H), 7.50 (d, J=8.0 Hz, 2H). LCMS: 696.5 (M+1).

### Synthesis of DTx-10-01

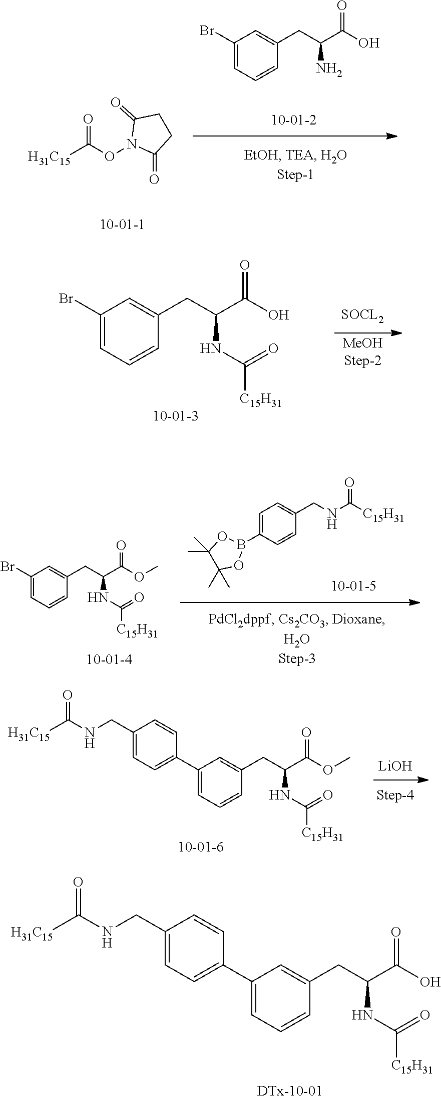

### Step 1:

To a stirred solution of compound 10-01-1 (5 g, 0.0141 mol) in 65% aq. ethanol (50 mL) was added Et₃N (10 mL, 0.0707 mol), compound 10-01-2 (3.45 g, 0.0141 mol) slowly at RT. The resulting mixture was stirred at 75° C. for 16 h. The reaction was monitored by LCMS. The reaction mixture was neutralized with 1.5 N HCl, precipitated solid was filtered, washed with water and dried to get the product 10-01-3 as an off white solid. (5.5 g, 80.6%)

### Step 2:

To a stirred solution of compound 10-01-3 (5.5 g, 0.0113 mol) in methanol (550 mL) was added thionyl chloride (22 mL) slowly at RT. The resulting mixture was stirred at RT for 16 h. The reaction was monitored by LCMS. The reaction mixture was concentrated under reduced pressure to get crude product which was triturated with diethyl ether to get pure compound of 10-01-4 as an off white solid (4.3 g, 76%).

### Step 3:

To a stirred solution of compound 10-01-4 (4.3 g, 0.0086 mol) in dioxane (90 mL) and water (9 mL) was added compound 10-01-5 (4.5 g, 0.00952 mol) and Cs₂CO₃ (8.4.6 g, 0.0259 mol). After degassing with nitrogen, Pd(dppf)Cl₂ (0.7 g, 0.0008 mol) was added to the reaction mixture. The resulting mixture was stirred at 90° C. for 3 h. The reaction mixture was monitored by LCMS, the reaction mixture was filtered through celite bed and concentrated under vacuum to give crude product which was further purified by column chromatography using 3% MeOH in DCM as eluent to get the product 10-01-6 as brown solid. (1.1 g, 16.68%)

### Step 4:

To a stirred solution of compound 10-01-6 (1.1 g, 0.0014 mol) in MeOH, THF (6.5 mL; 13 mL) and H₂O (6.5 mL) was added LiOH.H₂O (0.18 g, 0.0042 mol) and the reaction mixture was stirred at 50° C. for 3 h. The reaction mixture was monitored by LCMS, the reaction mixture was concentrated under vacuum. The resultant product was neutralized with 1.5 N HCl, the solid which was precipitated was filtered, washed with water and dried under vacuum to get the crude product. The crude product was triturated with MeOH to obtained pure DTx-10-01 as off white solid (0.7 g, 64%).

### Analytics of DTx-10-01

¹H-NMR- (400 MHz, TFA-d1): δ 0.78-0.80 (m, 6H), 1.13-1.45 (m, 50H), 1.73-1.75 (m, 2H), 2.39-2.43 (m, 1H), 2.70-2.74 (m, 2H), 3.14-3.20 (m, 1H), 3.46-3.51 (m, 2H), 4.68 (s, 2H), 5.17-5.20 (m, 1H), 7.17 (d, J=7.2 Hz, 1H), 7.33-7.43 (m, 4H), 7.50 (d, J=7.6 Hz, 1H), 7.57-7.58 (m, 2H). LCMS: 748.5 (M+1)

### Synthesis of DTx-11-01

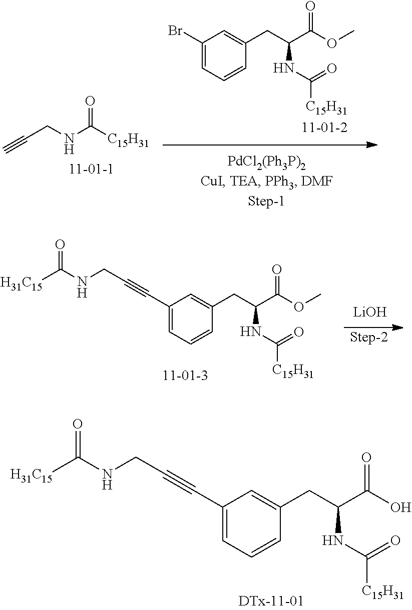

### Step 1:

To a stirred solution of compound 11-01-1 (2.68 g, 0.0091 mol) in DMF (35 mL) in a sealed tube was added compound 11-01-2 (3.5 g, 0.0070 mol), TEA (18 mL), PPh₃ (0.18 g, 0.0007 mol) and CuI (0.16 g, 0.0008 mol). After degassing with nitrogen, PdCl₂(Ph₃P)₂ (0.39 g, 0.0005 mol) was added to the reaction mixture. The resulting mixture was stirred at 110° C. for 3 h. The reaction mixture was monitored by LCMS, the reaction mixture was filtered through celite bed and concentrated under vacuum to give crude product which was further purified by column chromatography using 25% EtOAc in Hexane as eluent to get the product 11-01-3 as off white solid. (1 g, 20%)

### Step 2:

To a stirred solution of compound 11-01-3 (1 g, 0.0014 mol) in MeOH, THF (6.5 mL; 13 mL) and H₂O (6.5 mL) was added LiOH.H₂O (0.17 g, 0.0042 mol) and the reaction mixture was stirred at 50° C. for 2 h. The reaction mixture was monitored by LCMS, the reaction mixture was concentrated under vacuum to give crude which was neutralized with 1.5 N HCl, precipitated solid was filtered, washed with water and dried under vacuum to get the crude product. The crude product was further purified by column chromatography using 3% MeOH in DCM as eluent to get the product DTx-11-01 as off pale brown solid (0.7 g, 71%).

### Analytics of DTx-11-01

¹H-NMR- (400 MHz, TFA-d1): δ 0.87-0.90 (m, 6H), 1.31-1.47 (m, 48H), 1.65-1.68 (m, 2H), 1.81-1.85 (m, 2H), 2.71-2.74 (m, 2H), 2.89-2.95 (m, 2H), 3.42 (d, J=14.8 Hz, 1H), 3.57 (d, J=14.8 Hz, 1H), 4.50 (s, 2H), 5.20-5.24 (m, 1H), 7.25 (d, J=7.6 Hz, 1H), 7.34 (s, 1H), 7.39 (t, J=8.0 Hz, 1H), 7.47 (d, J=7.6 Hz, 1H). LCMS: 696.5 (M+1).

### Synthesis of DTx-04-01

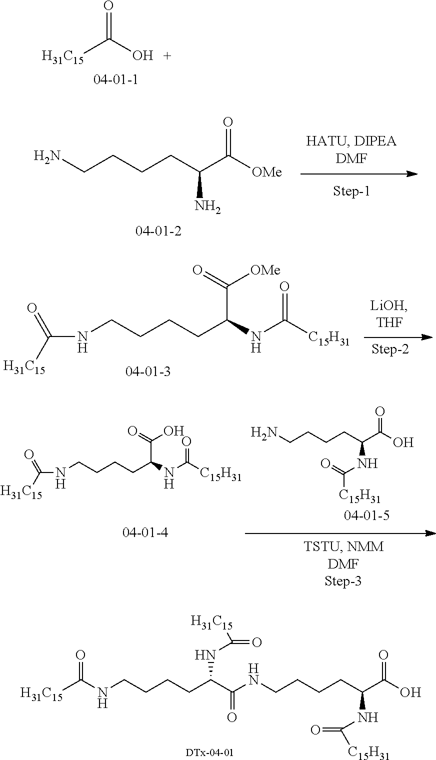

### Step 1:

To a stirred solution of compound 04-01-2 (5 g, 0.021 mol) in DMF (100 mL) was added DIPEA (19.7 mL, 0.107 mol), compound 04-01-1 (13.73 g, 0.053 mol) HATU (12.23 g, 0.032 mol) slowly at RT. The resulting mixture was stirred at RT for 16 h. The reaction was monitored by LCMS. The reaction mixture was quenched with ice cold water and filtered the solid, dried the solid under the vacuum to get the product 04-01-3 as off white solid (9.1 g, 67%).

### Step 2:

To a stirred solution of compound 04-01-3 (5 g, 0.0078 mol) in MeOH, THF (100 mL; 1:1) and H₂O (5 mL) was added LiOH.H₂O (0.660 g, 0.0157 mol) and the reaction mixture was stirred at RT for 16 h. The reaction mixture was monitored by LCMS, the reaction mixture was concentrated under vacuum to give crude which was neutralized with 1.5 N HCl, the solid which was precipitated was filtered, washed with water and dried under vacuum to get the product 04-01-4 as off white solid (3.9 g, 80%).

### Step 3:

To a stirred solution of compound 04-01-4 (3.0 g, 0.0048 mol) in DMF (60 mL) was added NMM (15 mL), followed by TSTU (2.18 g, 0.0096 mol) at RT. The resulting mixture was stirred at RT for 2 h. Compound 5 (3.69 g, 0.0096 mol) was added to the reaction mixture at 0° C. and then stirred at RT for 16 h. The reaction mixture was neutralized with 1.5 N HCl, precipitated solid was filtered, washed with water and dried. The crude product was triturated with MeOH to get the product DTx-04-01 as an off white solid. (2.8 g, 58%).

### Analytics of DTx-04-01

¹H-NMR- (400 MHz, TFA-d): δ 1.09-1.13 (m, 9H), 1.57-2.16 (m, 84H), 2.38-2.44 (m, 3H), 2.77-2.94 (m, 4H), 3.18-3.31 (m, 5H), 3.69-3.81 (m, 5H), 4.87-4.92 (m, 1H). LCMS: 990.8 (M+1).

### Synthesis of DTx-05-01

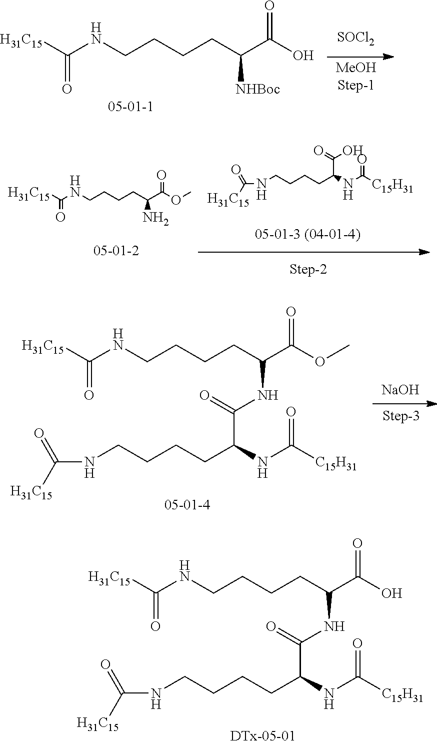

### Step 1:

To a stirred solution of compound 05-01-1 (5 g, 0.0103 mol) in methanol (50 mL) was added thionyl chloride (3.8 mL, 0.0516 mol) slowly at 0° C. The resulting mixture was stirred at RT for 16 h. The resulting mixture was evaporated and triturated with diethyl ether to give compound 05-01-2 as an off white solid which was directly proceeded for next step (3.5 g, 85%).

### Step 2:

To a stirred solution of compound 05-01-2 (2.89 g, 0.0067 mol) in DMF (35 mL) was added DIPEA (1.55 mL, 0.0084 mol), compound 05-01-3 (3.5 g, 0.0056 mol) and HBTU (2.12 g, 0.0056 mol) slowly at 0° C. The resulting mixture was stirred at 50° C. for 16 h. The reaction was monitored by LCMS. The reaction mixture was neutralized with 1.5 N HCl, precipitated solid was filtered, washed with water and dried to give compound 05-01-4 as pale brown solid. (3.2 g, 69%).

### Step 3:

To a stirred solution of compound 05-01-4 (3.2 g, 0.0031 mol) in MeOH, THF (60 mL; 1:1) and H₂O (3 mL) was added NaOH (0.25 g, 0.0062 mol) and the reaction mixture was stirred at 50° C. for 16 h. The reaction mixture was monitored by LCMS, the reaction mixture was concentrated and neutralized with 1.5 N HCl. The precipitated solid was filtered, washed with water and dried. The crude product was triturated with MeOH to give DTx-05-01 as pale brown solid. (2.3 g, 73%).

### Analytics of DTx-05-01

¹H-NMR- (400 MHz, TFA-d): δ 0.87-0.89 (m, 9H), 1.60-1.80 (m, 76H), 1.94-2.14 (m, 15H), 2.55-2.59 (m, 2H), 2.70-2.75 (m, 4H), 3.59-3.60 (m, 4H), 4.73-4.76 (m, 1H). LCMS: 990.8 (M+1).

### Synthesis of DTx-01-50 & DTx-01-52

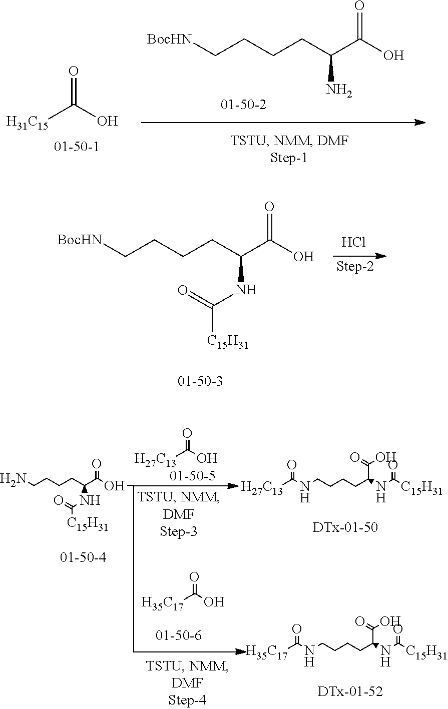

### Step 1:

To a stirred solution of 01-50-1 (5.0 g, 0.019 mol) in DMF (50 mL) was added NMM (25 mL), followed by TSTU (6.46 g, 0.021 mol) at RT. The resulting mixture was stirred at RT for 2 h. 01-50-2 (7.2 g, 0.029 mol) was added to the reaction mixture at 0° C. and then stirred at 70° C. for 5 h and then concentrated. The residue was neutralized with 1.5 N HCl, precipitated solid was filtered, washed with water and dried. The crude product was triturated with MeOH to get the product 01-50-3 as brown solid. (9.1 g, 96%).

### Step 2:

To a stirred solution of compound 01-50-3 (9.1 g, 0.018 mol) in 1,4 dioxane (45 mL) was added 4 M HCl in dioxane (45 mL) slowly at RT. The resulting mixture was stirred at RT for 16 h. The reaction mixture was concentrated under reduced pressure to get crude product which was triturated with diethyl ether to get pure compound of 01-50-4 as an off white solid (6.5 g, 82%).

### Step 3:

To a stirred solution of compound 01-50-5 (1.5 g, 0.0065 mol) in DMF (45 mL) was added NMM (23 mL), followed by TSTU (2.17 g, 0.0072 mol) at RT. The resulting mixture was stirred at RT for 2 h. 01-50-4 (3.32 g, 0.0078 mol) was added to the reaction mixture at 0° C. and then stirred at 70° C. for 5 h and then concentrated. The residue was neutralized with 1.5 N HCl, precipitated solid was filtered, washed with water and dried. The crude product was triturated with MeOH to get the product DTx-01-50 as pale brown solid. (2.1 g, 53%). LCMS: 595.5 (M+1). ¹H-NMR- (400 MHz, TFA-d): δ 0.93-0.95 (m, 6H), 1.38-1.65 (m, 44H), 1.65-1.69 (m, 2H), 1.84-2.06 (m, 7H), 2.20-2.24 (m, 1H), 2.67 (t, J=7.6 Hz, 2H), 2.82 (t, J=7.9 Hz, 2H), 3.68 (t, J=6.8 Hz, 2H), 4.93 (t, J=8.0 Hz, 1H).

### Step 4:

To a stirred solution of compound 6 (1.5 g, 0.0052 mol) in DMF (45 mL) was added NMM (23 mL), followed by TSTU (1.74 g, 0.0058 mol) at RT. The resulting mixture was stirred at RT for 2 h. Compound 4 (2.66 g, 0.0063 mol) was added to the reaction mixture at 0° C. and then stirred at 70° C. for 5 h and then concentrated. The residue was neutralized with 1.5 N HCl, precipitated solid was filtered, washed with water and dried. The crude product was triturated with MeOH to get the product DTx-01-52 as pale brown solid. (2.2 g, 64%). LCMS: 652.5 (M+1).

¹H-NMR- (400 MHz, TFA-d): δ 0.93-0.94 (m, 6H), 1.37-1.59 (m, 52H), 1.66-1.68 (m, 2H), 1.84-2.05 (m, 7H), 2.20-2.23 (m, 1H), 2.67 (t, J=7.3 Hz, 2H), 2.81 (t, J=7.5 Hz, 2H), 3.69 (t, J=6.2 Hz, 2H), 4.92 (t, J=4.9 Hz, 1H).

### Synthesis of DTx-01-51 & DTx-01-54

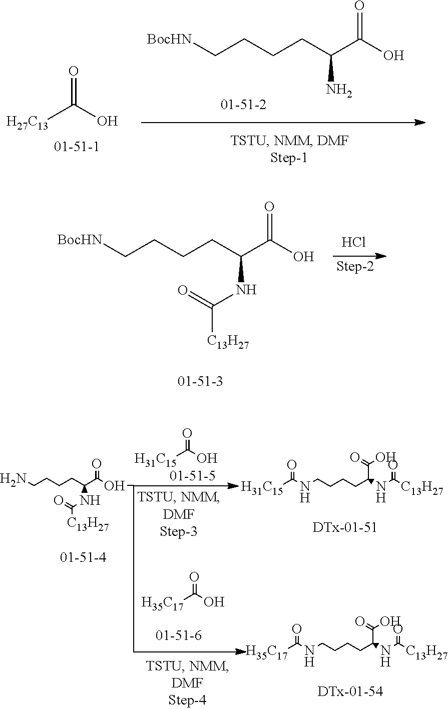

### Step 1:

To a stirred solution of 01-51-1 (5.0 g, 0.021 mol) in DMF (50 mL) was added NMM (25 mL), followed by TSTU (7.25 g, 0.024 mol) at RT. The resulting mixture was stirred at RT for 2 h. Compound 01-51-2 (8.09 g, 0.032 mol) was added to the reaction mixture at 0° C. and then stirred at 70° C. for 5 h and then concentrated. The residue was neutralized with 1.5 N HCl, precipitated solid was filtered, washed with water and dried. The crude product was triturated with MeOH to get the product 01-51-3 as brown solid. (9 g, 90%).

### Step 2:

To a stirred solution of compound 01-51-3 (9 g, 0.014 mol) in 1,4 dioxane (45 mL) was added 4 M HCl in dioxane (45 mL) slowly at RT. The resulting mixture was stirred at RT for 16 h. The reaction mixture was concentrated under reduced pressure to get crude product which was triturated with diethyl ether to get pure compound of 01-51-4 as an off white solid (6.6 g, 81%).

### Step 3:

To a stirred solution of compound 01-51-5 (1.5 g, 0.0058 mol) in DMF (45 mL) was added NMM (23 mL), followed by TSTU (1.93 g, 0.0064 mol) at RT. The resulting mixture was stirred at RT for 2 h. Compound 01-51-4 (2.76 g, 0.0070 mol) was added to the reaction mixture at 0° C. and then stirred at 70° C. for 5 h and then concentrated. The residue was neutralized with 1.5 N HCl, precipitated solid was filtered, washed with water and dried. The crude product was triturated with MeOH to get the product DTx-01-51 as pale brown solid. (2.4 g, 68%). LCMS: 595.5 (M+1). ¹H-NMR- (400 MHz, TFA-d): δ 0.89-0.92 (m, 6H), 1.34-1.50 (m, 44H), 1.63-1.65 (m, 2H), 1.81-2.08 (m, 7H), 2.20-2.21 (m, 1H), 2.63 (t, J=7.3 Hz, 2H), 2.78 (t, J=7.4 Hz, 2H), 3.65 (t, J=6.4 Hz, 2H), 4.89 (t, J=7.1 Hz, 1H).

### Step 4:

To a stirred solution of compound 01-51-6 (1.5 g, 0.0052 mol) in DMF (45 mL) was added NMM (23 mL), followed by TSTU (1.74 g, 0.0058 mol) at RT. The resulting mixture was stirred at RT for 2 h. Compound 01-51-4 (2.49 g, 0.0063 mol) was added to the reaction mixture at 0° C. and then stirred at 70° C. for 5 h and then concentrated. The residue was neutralized with 1.5 N HCl, precipitated solid was filtered, washed with water and dried. The crude product was triturated with MeOH to get the product DTx-01-54 as pale brown solid. (2.2 g, 66%). LCMS: 624.6 (M+1).

¹H-NMR- (400 MHz, TFA-d): δ 0.89-0.90 (m, 6H), 1.32-1.57 (m, 49H), 1.62-1.64 (m, 2H), 1.74-1.99 (m, 6H), 2.14-2.18 (m, 1H), 2.61 (t, J=7.6 Hz, 2H), 2.76 (t, J=7.6 Hz, 2H), 3.62 (t, J=7.0 Hz, 2H), 4.85-4.88 (m, 1H).

### Synthesis of DTx-01-53 & DTx-01-55

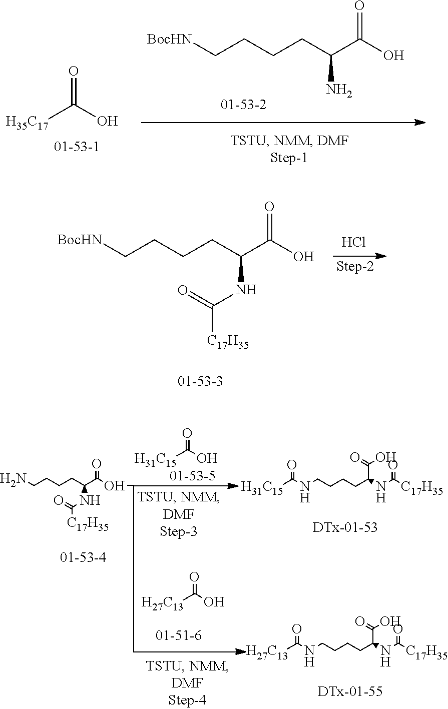

### Step 1:

To a stirred solution of compound 1 (5.0 g, 0.017 mol) in DMF (50 mL) was added NMM (25 mL), followed by TSTU (5.82 g, 0.019 mol) at RT. The resulting mixture was stirred at RT for 2 h. Compound 2 (5.18 g, 0.021 mol) was added to the reaction mixture at 0° C. and then stirred at 70° C. for 5 h and then concentrated. The reaction mixture was neutralized with 1.5 N HCl, precipitated solid was filtered, washed with water and dried. The crude product was triturated with MeOH to get the product 3 as brown solid. (8.6 g, 95%).

### Step 2:

To a stirred solution of compound 3 (8.6 g, 0.016 mol) in 1,4 dioxane (43 mL) was added 4 M HCl in dioxane (43 mL) slowly at RT. The resulting mixture was stirred at RT for 16 h. The reaction mixture was concentrated under reduced pressure to get crude product which was triturated with diethyl ether to get pure compound of 4 as an off white solid (7 g, 93%).

### Step 3:

To a stirred solution of compound 5 (1.5 g, 0.0058 mol) in DMF (45 mL) was added NMM (23 mL), followed by TSTU (1.94 g, 0.0064 mol) at RT. The resulting mixture was stirred at RT for 2 h. Compound 4 (3.15 g, 0.0070 mol) was added to the reaction mixture at 0° C. and then stirred at 70° C. for 5 h and then concentrated. The reaction mixture was neutralized with 1.5 N HCl, precipitated solid was filtered, washed with water and dried. The crude product was triturated with MeOH to get the product DTx-01-53 as pale brown solid. (2.2 g, 57%). LCMS: 652.6 (M+1). ¹H-NMR- (400 MHz, TFA-d): δ 0.82-0.85 (m, 6H), 1.27-1.50 (m, 52H), 1.54-1.58 (m, 2H), 1.73-1.94 (m, 7H), 2.07-2.14 (m, 1H), 2.56 (t, J=8.0 Hz, 2H), 2.71 (t, J=8.0 Hz, 2H), 3.58 (t, J=6.8 Hz, 2H), 4.81-4.84 (m, 1H).

### Step 4:

To a stirred solution of compound 6 (1.5 g, 0.0065 mol) in DMF (45 mL) was added NMM (23 mL), followed by TSTU (2.17 g, 0.0072 mol) at RT. The resulting mixture was stirred at RT for 2 h. Compound 4 (3.53 g, 0.0078 mol) was added to the reaction mixture at 0° C. and then stirred at 70° C. for 5 h and then concentrated. The residue was neutralized with 1.5 N HCl, precipitated solid was filtered, washed with water and dried. The crude product was triturated with MeOH to get the product DTx-01-55 as pale brown solid. (2.3 g, 56%). LCMS: 624.6 (M+1).

¹H-NMR- (400 MHz, TFA-d): δ 0.90-0.93 (m, 6H), 1.35-1.49 (m, 48H), 1.60-1.63 (m, 2H), 1.77-2.02 (m, 7H), 2.17-2.21 (m, 1H), 2.64 (t, J=7.6 Hz, 2H), 2.78 (t, J=7.7 Hz, 2H), 3.65 (t, J=7.0 Hz, 2H), 4.88-4.91 (m, 1H).

### Synthesis of the Methyl Ester of Lipid Motif DTx-01-102 (DTx-01-102-OMe)

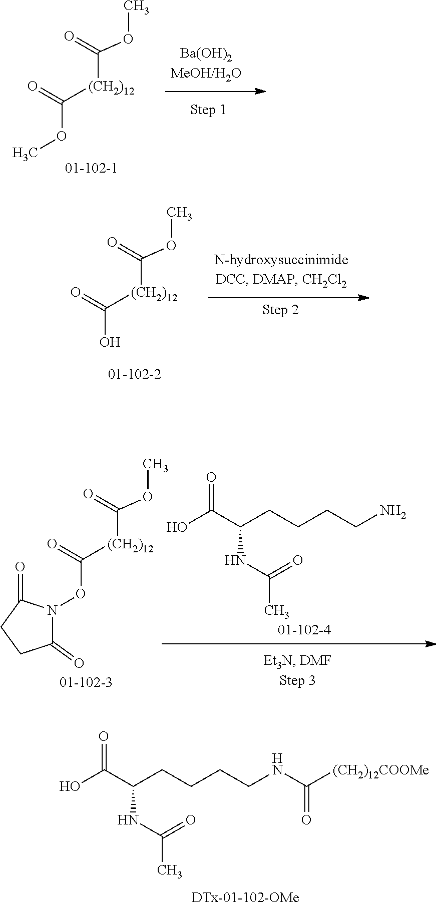

### Step 1: Synthesis of Intermediate 01-102-2

To a stirred solution of 01-102-1 (10 g, 34.9 mmol) in MeOH (25 mL) at RT was added slowly Ba(OH)₂ (3.0 g, 17.4 mmol). The resulting mixture was stirred at RT. After 48 h, the reaction mixture was quenched with ice water, acidified with 1.5 M HCl, and extracted with EtOAc. The combined organic extract was washed with water, brine, dried over Na₂SO₄, and then evaporated to yield crude 01-102-2. Purification by column chromatography (15% EtOAc in petroleum ether) afforded 01-102-2 as an off-white solid (5.7 g, 60.0%).

### Step 2: Synthesis of Intermediate 01-102-3

To a stirred solution of 01-102-2 (5.7 g, 20.9 mmol) in DCM (250 mL) at RT was added DMAP (0.257 g, 2.1 mmol) and DCC (6.48 g, 31.4 mmol), followed by N-hydroxysuccinimide (2.4 g, 20.9 mmol). The resulting mixture was stirred at RT. After 16 h, the reaction mixture was filtered through a sintered funnel. The filtrate was evaporated to yield crude 01-102-3 as a pale yellow oil (5.0 g, 65%), which was used directly in the next step without further purification.

### Step 3: Synthesis of Lipid Motif DTx-01-102

To a stirred solution of 01-102-4 (2.54 g, 13.5 mmol) in DMF (100 mL) at RT was added slowly Et₃N (5.6 mL, 40.5 mmol), then 01-102-3 (5.0 g, 13.5 mmol). The resulting mixture was stirred at RT. After 16 h, the reaction mixture was quenched with ice water dropwise and extracted with EtOAc. The combined organic extract was washed with ice water, brine, dried over Na₂SO₄, and then evaporated to yield crude DTx-01-102-OMe. Purification by column chromatography (3% MeOH in DCM) afforded the methyl ester of lipid motif DTx-01-102 (i.e., DTx-01-102-OMe) as an off-white solid (2 g, 33.4%). LCMS m/z (M+H)⁺: 443.3; ¹H-NMR (400 MHz, TFA): δ 1.30-1.51 (m, 17H), 1.60-1.85 (m, 8H), 1.99 (m, 0.5H), 2.22-2.24 (m, 2H), 2.39 (s, 1H), 2.48-2.51 (m, 2H), 2.74-2.79 (m, 4H), 3.62-3.65 (m, 2H), 3.86 (s, 3H), 4.86-4.88 (m, 0.5 H).

### Synthesis of the Methyl Ester of Lipid Motif DTx-01-103 (DTx-01-103-OMe)

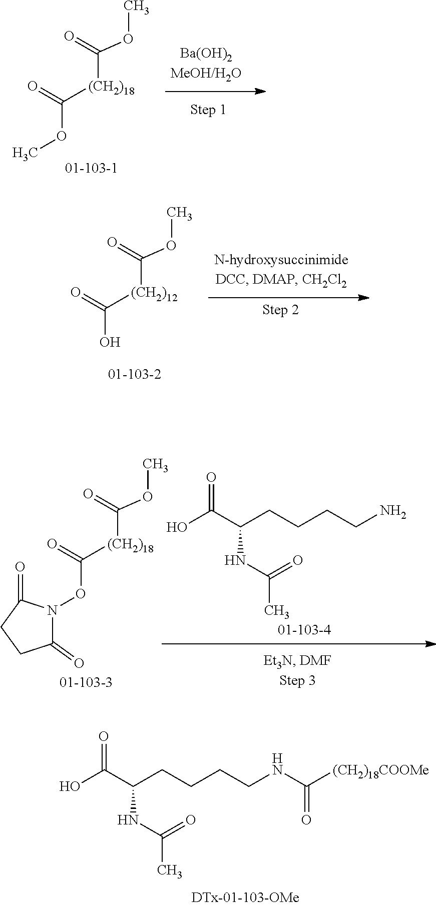

### Step 1: Synthesis of Intermediate 01-103-2

To a stirred solution of 01-103-1 (3 g, 8.76 mmol) in MeOH (15 mL) at RT was added slowly Ba(OH)₂ (0.75 g, 4.4 mmol). The resulting mixture was stirred at RT. After 48 h, the reaction mixture was quenched with ice water, acidified with 1.5 M HCl, and extracted with EtOAc. The combined organic extract was washed with water, brine, dried over Na₂SO₄, and then evaporated to yield crude 01-103-2. Purification by column chromatography (10% EtOAc in petroleum ether) afforded 01-103-2 as an off-white solid (2.5 g, 86.8%).

### Step 2: Synthesis of Intermediate 01-103-3

To a stirred solution of 01-103-2 (2.5 g, 7.6 mmol) in DCM (100 mL) at RT was added DMAP (0.093 g, 0.76 mmol) and DCC (2.35 g, 11.4 mmol), followed by N-hydroxysuccinimide (0.87 g, 7.6 mmol). The resulting mixture was stirred at RT. After 16 h, the reaction mixture was filtered through a sintered funnel. The filtrate was evaporated to yield crude 01-103-3 as a pale yellow oil (3.4 g, ˜100%), which was used directly in the next step without further purification.

### Step 3: Synthesis of Lipid Motif DTx-01-103

To a stirred solution of 01-103-4 (1.49 g, 7.9 mmol) in DMF (60 mL) at RT was added slowly Et₃N (3.3 mL, 23.7 mmol), then 01-103-3 (3.4 g, 7.9 mmol). The resulting mixture was stirred at RT. After 16 h, the reaction mixture was quenched with ice water dropwise and extracted with EtOAc. The combined organic extract was washed with ice water, brine, dried over Na₂SO₄, and then evaporated to yield crude DTx-01-103-OMe. Purification by column chromatography (3% MeOH in DCM) afforded the methyl ester of lipid motif DTx-01-103 (i.e., DTx-01-103-OMe) as an off-white solid (2.1 g, 53.3%). LCMS m/z (M+H)⁺: 499.4; ¹H-NMR (400 MHz, TFA): δ 1.40-1.70 (m, 24H), 1.71-1.80 (m, 2H), 1.81-1.90 (m, 2H), 1.91-2.05 (m, 4H), 2.1-2.2 (m, 1H), 2.25-2.4 (m, 1H), 2.55 (s, 3H), 2.57-2.7 (m, 3H), 2.85-2.95 (m, 2H), 3.72-3.82 (m, 2H), 3.99 (s, 3H), 5.0-5.1 (m, 1H).

### Synthesis of the Methyl Ester of Lipid Motif DTx-01-104 (DTx-01-104-OMe)

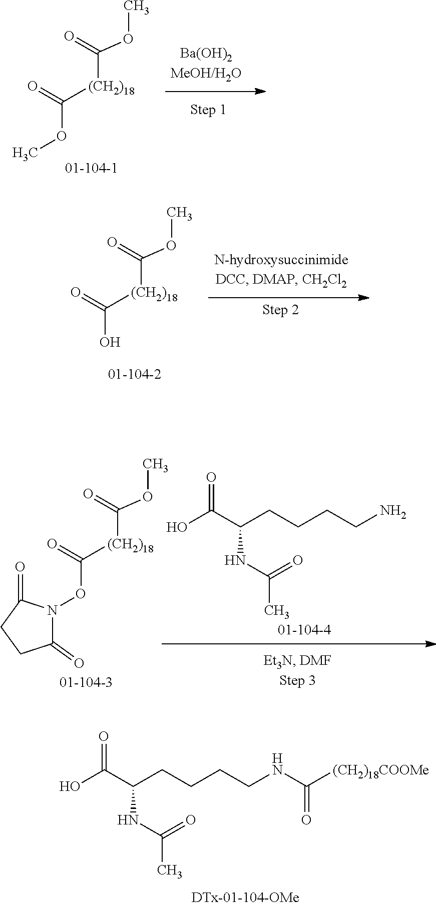

### Step 1: Synthesis of Intermediate 01-104-2

To a stirred solution of 01-104-1 (5 g, 13.5 mmol) in MeOH (30 mL) at RT was added slowly Ba(OH)₂ (2.3 g, 13.5 mmol). The resulting mixture was stirred at 50° C. After 48 h, the reaction mixture was quenched with ice water, acidified with 1.5 M HCl, and extracted with EtOAc. The combined organic extract was washed with water, brine, dried over Na₂SO₄, and then evaporated to yield crude 01-104-2. Purification by column chromatography (10% EtOAc in petroleum ether) afforded 01-104-2 as an off-white solid (4.1 g, 85%).

### Step 2: Synthesis of Intermediate 01-104-3

To a stirred solution of 01-104-2 (4.1 g, 11.5 mmol) in DCM (120 mL) at RT was added DMAP (0.140 g, 1.15 mmol) and DCC (3.55 g, 17.2 mmol), followed by N-hydroxysuccinimide (1.3 g, 11.5 mmol). The resulting mixture was stirred at RT. After 16 h, the reaction mixture was filtered through a sintered funnel. The filtrate was evaporated to yield crude 01-104-3 as a pale yellow liquid (3.7 g, 71%), which was used directly in the next step without further purification.

### Step 3: Synthesis of Lipid Motif DTx-01-104

To a stirred solution of 01-104-4 (1.32 g, 7.02 mmol) in DMF (50 mL) at RT was added slowly Et₃N (2.94 mL, 21.1 mmol), 01-104-3 (3.7 g, 7.02 mmol). The resulting mixture was stirred at RT. After 16 h, the reaction mixture was quenched with ice water dropwise and extracted with EtOAc. The combined organic extract was washed with ice water, brine, dried over Na₂SO₄, and then evaporated to yield crude DTx-01-104-OMe. Purification by column chromatography (3% MeOH in DCM) afforded the methyl ester of lipid motif DTx-01-104 (i.e., DTx-01-104-OMe) as an off-white solid (2.3 g, 53.6%). LCMS m/z (M+H)⁺: 527.3; ¹H-NMR (400 MHz, DMSO-d6): δ 1.25-1.52 (m, 30H), 1.55-1.90 (m, 9H), 1.95-1.99 (m, 1H), 2.10-2.30 (m, 1.5H), 2.38 (s, 3H), 2.45-2.49 (m, 2H), 2.72-2.81 (m, 3H), 3.60-3.63 (m 2H), 3.83 (s, 3H), 4.82-4.85 (m, 1H).

The motifs presented in the above synthesis schemes, as well as additional motifs, are listed in the tables provided herein.

The synthesis of certain motifs produces a motif comprising a methyl ester protecting group. For example, synthesis of uptake domain DTx-01-12 produces DTx-01-12-OMe, the methyl ester of DTx-01-12. Following conjugation to a nucleic acid compound, the methyl ester protecting group is removed and no longer present in the uptake domain. Thus, as illustrated in the tables and figures herein, these certain domains are shown without a methyl ester protecting group.

### Conjugating the Lipid Motifs to Double-Stranded Oligonucleotides

As described in Schemes I, II, III, and IV below, various motifs were conjugated to siRNA. Table 5 below provides the siRNAs. Within the sequences given, the designations “m”, “e”, “f”, and “*” denote 2′-O-methyl residues, 2′-O-methyoxyethyl residues, 2′-deoxy-2′-fluoro residues, and phosphorothioate linkages, respectively. PO₄ denotes a terminal phosphate group.

TABLE 5
siRNA Molecules

| siRNA | | |
|---|---|---|
| Name | siRNA Properties | |
| DTxO- | Target | Sense Sequence (5′ to 3′) |
| 0003 | PTEN | fG mA fU mG fA mU fG mU fU fU fG |
| | | mA fA mA fC mU fA mU fU*T*T |
| | | (SEQ ID NO: 1) |
| | | Antisense Sequence (5′ to 3′) |
| | | PO₄-mA fA mU fA mG fU mU fU mC mA |
| | | mA fA mC fA mU fC mA fU mC*T*T |
| | | (SEQ ID NO: 2) |
| DTxO- | Target | Sense Sequence (5′ to 3′) |
| 0038 | PTEN | fA*mC*fC mU fG mA fU mC fA mU fU |
| | | mA fU mA fG mA fU*mA*fA |
| | | (SEQ ID NO: 3) |
| | | Antisense Sequence (5′ to 3′) |
| | | PO₄-eT*fU*mA fU mC fU mA fU mA fA |
| | | mU fG mA fU mC fA mG fG mU *T *T |
| | | (SEQ ID NO: 4) |

siRNA compounds were conjugated to HLEM and/or uptake motifs according to Scheme I, II, III, or IV as appropriate. The structures, including the site of attachment on the sense strand (5′ end or 3′ end) are shown in FIG. 1.

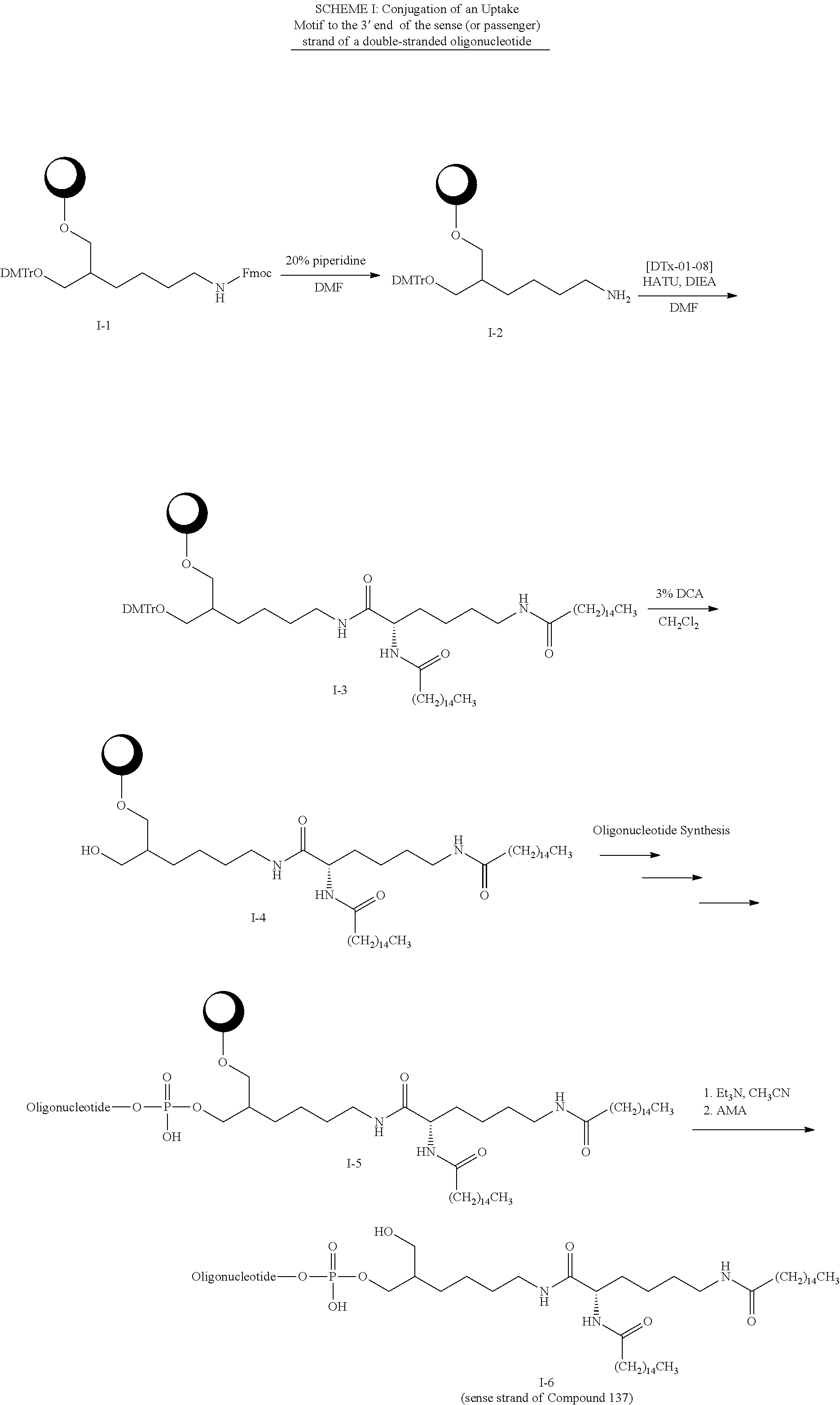

Scheme I above illustrates the preparation of a sense strand of a double-stranded oligonucleotide conjugated with a lipid moiety at the 3′ end of the sense strand, using the sense strand of Compound DT-000137 as an example. In summary, 3′-amino CPG beads I-1 (Glen Research, Catalog No. 20-2958) modified with the DMT and Fmoc-protected C7 linker illustrated above were treated with 20% piperidine/DMF to afford Fmoc-deprotected amino C7 CPG beads I-2. DTx-01-08 was then coupled to I-2 using HATU and DIEA in DMF to produce lipid-loaded CPG beads I-3, which were treated by 3% dichloroacetic acid (DCA) in DCM to remove the DMT protecting group and afford I-4. Oligonucleotide synthesis of the sense strand of DTxO-0003 on I-4 was accomplished via standard phosphoramidite chemistry and yielded modified oligonucleotide-bounded CPG beads I-5. Subsequent treatment of I-5 with first triethylamine in acetonitrile, then AMA [ammonium hydroxide (28%)/methylamine (40%) (1:1 v/v)], deprotected and cleaved the DTx-01-08-conjugated oligonucleotide from the beads to yield crude I-6. This compound was then purified by RP-HPLC and characterized by MALDI-TOF MS using the [M+H] peak.

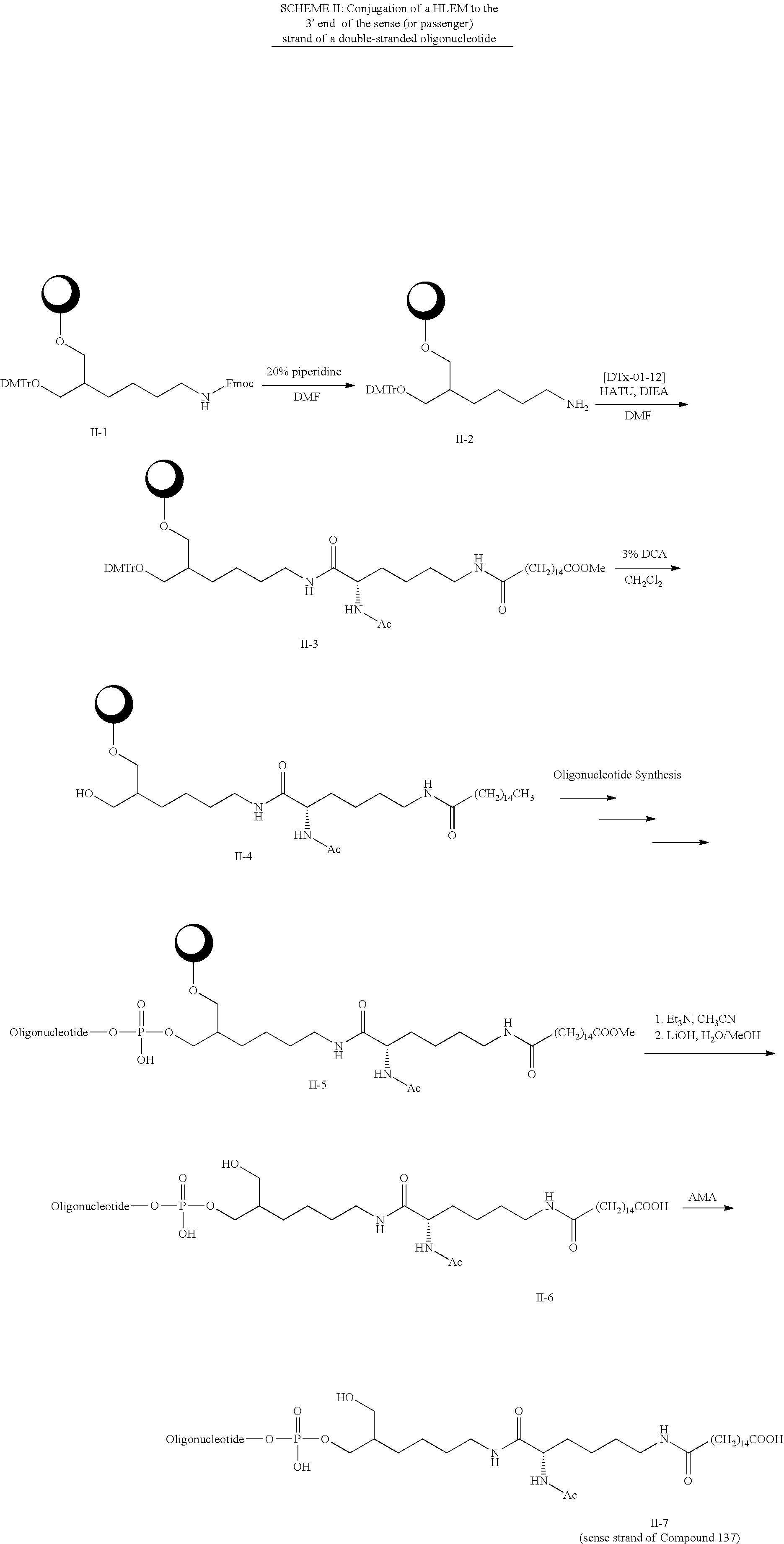

Scheme II above illustrates the preparation of a sense strand of a double-stranded oligonucleotide conjugated with a HLEM fatty-acid at the 3′ end, using the sense strand of Compound DT-000347 as an example. In summary, 3′-amino CPG beads II-1 (Glen Research, Catalog No. 20-2958) modified with the DMT and Fmoc-protected C7 linker illustrated above were treated with 20% piperidine/DMF to afford Fmoc-deprotected amino C7 CPG beads II-2. The methyl-ester protected DTx-01-12 was then coupled to II-2 using HATU and DIEA in DMF to produce the fatty-acid loaded CPG beads II-3, which were subsequently treated with 3% dichloroacetic acid (DCA) in DCM to remove the DMT protecting group and afford II-4. Oligonucleotide synthesis of the sense strand of the DTxO-0038 siRNA was performed on II-4 via standard phosphoramidite chemistry to generate II-5. At this point, the CPG-bound oligo is first treated with triethylamine in acetonitrile to remove the phosphate protecting groups, and then with 0.5 M LiOH in 3:1 v/v methanol/water to hydrolyze the terminal methyl ester. Subsequent treatment of II-6 with AMA [ammonium hydroxide (28%)/methylamine (40%) (1:1, v/v)] releases the conjugated oligonucleotide from the support and hydrolyzes all remaining base protecting groups to yield crude II-7, the sense strand of Compound DT-000347. This compound was then purified by RP-HPLC and characterized by MALDI-TOF MS by observing the [M+H] peak.

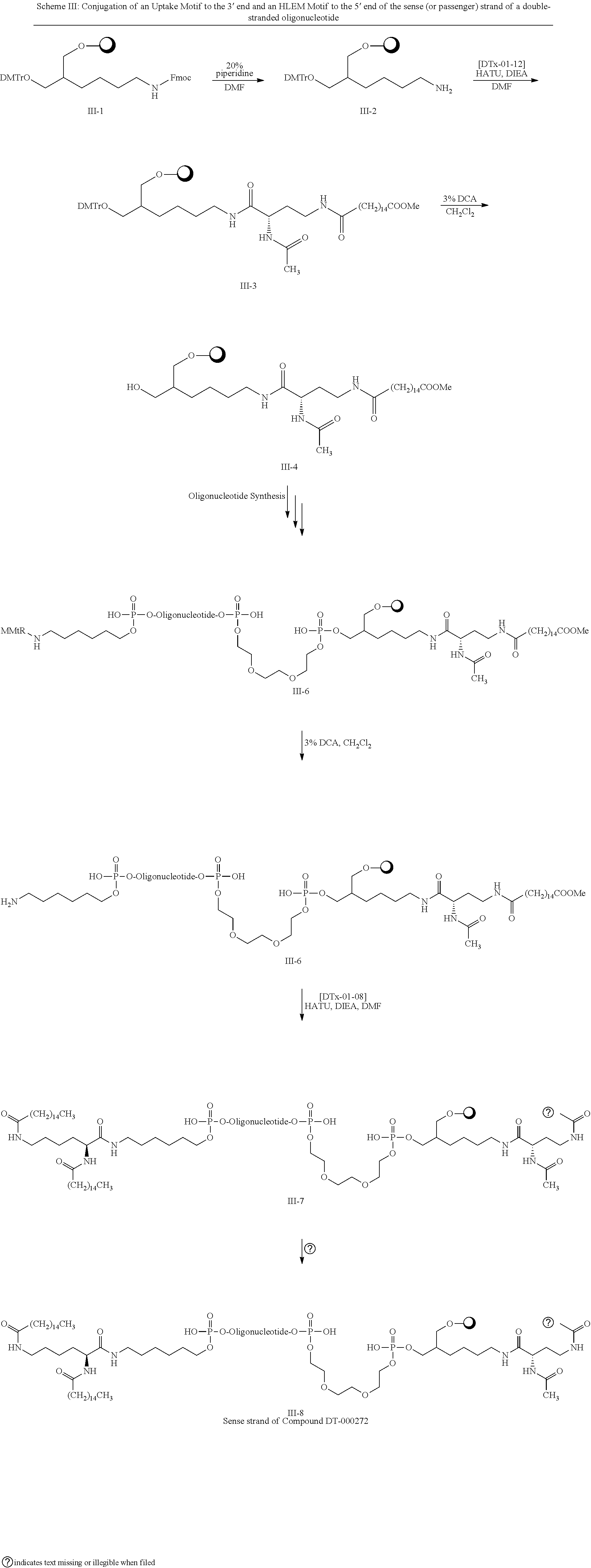

Scheme III above illustrates the preparation of a sense strand of a double-stranded oligonucleotide conjugated with a fatty-acid uptake motif at the 5′ end and a HLEM fatty-acid at the 3′ end, using the sense strand of Compound DT-000272 as an example. In summary, 3′-amino CPG beads III-1 (Glen Research, Catalog No. 20-2958) modified with the DMT and Fmoc-protected C7 linker illustrated above were treated with 20% piperidine/DMF to afford Fmoc-deprotected amino C7 CPG beads III-2. The methyl-ester protected DTx-01-12 was then coupled to III-2 using HATU and DIEA in DMF to produce the fatty-acid loaded CPG beads III-3, which were subsequently treated with 3% dichloroacetic acid (DCA) in DCM to remove the DMT protecting group and afford III-4. Oligonucleotide synthesis of the sense strand of the DTxO-0038 siRNA was performed on III-4 via standard phosphoramidite chemistry. The final coupling was with a phosphoramidite (Glen Research, Catalog No. 10-1906) that incorporated a monomethoxytrityl (MMTr) protected 6-carbon alkyl amine as shown in structure III-5. After removal of MMT with 3% dichloroacetic acid (DCA) in DCM, III-6 was coupled to DTx-01-08 using HATU and DIEA in DMF to yield III-7. Stepwise deprotection with triethylamine in acetonitrile (to remove phosphate protecting groups), 0.5 M LiOH in 3:1 v/v methanol/water (to hydrolyze the methyl ester), and AMA [ammonium hydroxide (28%)/methylamine (40%) (1:1, v/v)] (to remove base protecting groups and cleave the oligonucleotide from the synthesis resin) yielded crude III-8. Purification using RP-HPLC yielded a sense strand that could be used for Compound DT-000272. Purity and identity of III-8 were confirmed by analytical RP-HPLC and MALDI-TOF MS using the [M+H] peak, respectively.

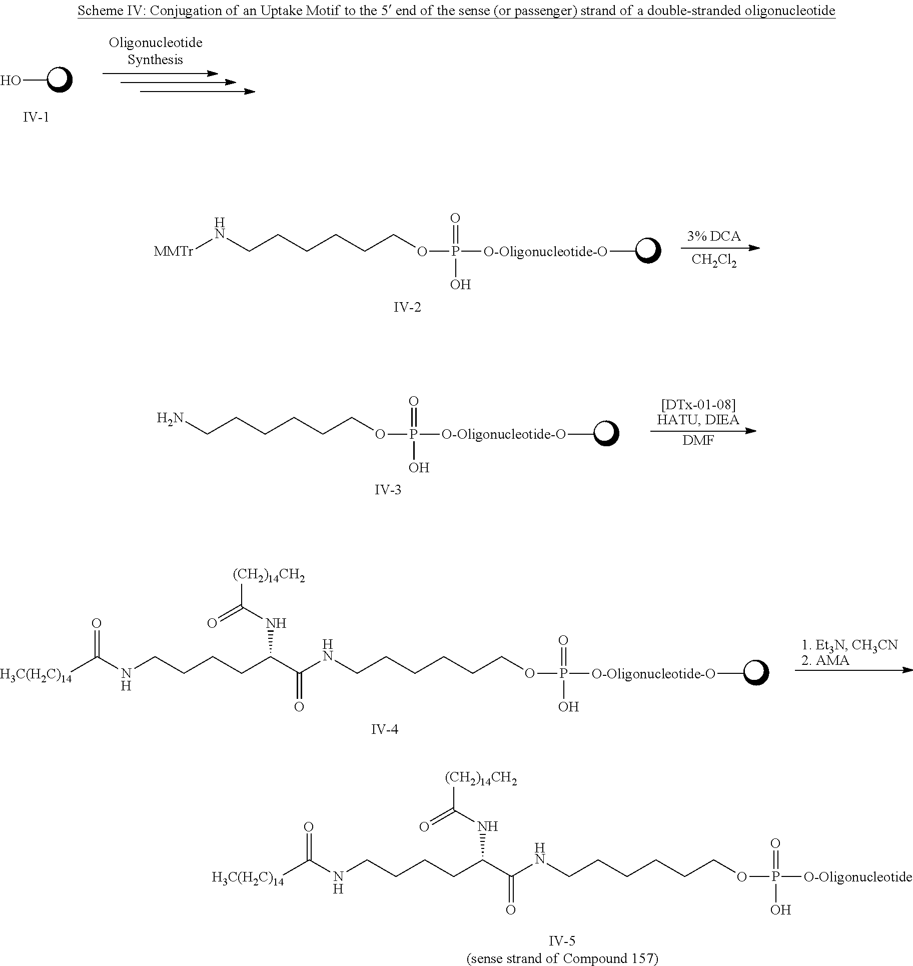

Scheme IV above illustrates the preparation of a sense strand of a double-stranded oligonucleotide conjugated with a fatty-acid uptake motif at the 5′ end, using the sense strand of Compound DT-000157 as an example. In summary, oligonucleotide synthesis was performed on CPG beads IV-1 (Glen Research, Catalog No. 20-5041-xx) via standard phosphoramidite chemistry. The final coupling was with a phosphoramidite (Glen Research, Catalog No. 10-1906) that incorporated a monomethoxytrityl (MMTr) protected 6-carbon alkyl amine as shown in structure IV-2. The MMTr was removed with 3% dichloroacetic acid (DCA) in DCM to yield IV-3. The free alkyl amine was coupled to DTx-01-08 using HATU and DIEA in DMF to yield IV-4. Stepwise deprotection with triethylamine in acetonitrile (to remove phosphate protecting groups) and AMA [ammonium hydroxide (28%)/methylamine (40%) (1:1, v/v)] (to remove base protecting groups and cleave the oligonucleotide from the synthesis resin) yielded crude IV-5. Purification using RP-HPLC yielded a sense strand that could be used for Compound 157. Purity and identity of IV-5 were confirmed by analytical RP-HPLC and MALDI-TOF MS using the [M+H] peak, respectively.

### Duplex Formation

For each of the sense strands synthesized by Schemes I, II, III, or IV and listed above, the complementary antisense strand was prepared via standard phosphoramidite chemistry, purified by IE-HPLC, and characterized by MALDI-TOF MS using the [M+H] peak. The duplex was formed by mixing equal molar equivalents of the sense strand and antisense strand, heating to 90° C. for 5 minutes, and then slowly cooling to room temperature. Duplex formation was confirmed by non-denaturing PAGE.

### Biological Data

### General Procedures and Methods

The following examples should not, of course, be construed as specifically limiting. Variations of these examples within the scope of the claims are within the purview of one skilled in the art and are considered to fall within the scope of the embodiments as described and claimed herein. The reader will recognize that the skilled artisan, armed with the present disclosure, and skill in the art is able to prepare and use the invention without exhaustive examples.

### Cell Culture

HEK293 cells were purchased from ATCC and cultured in DMEM containing 10% Fetal Bovine Serum (FBS), 2 mM L-glutamine, 1× non-essential amino acids, 100 U/mL penicillin and 100 mg/mL streptomycin in a humidified 37 C incubator with 5% CO2.

HUVEC cells were purchased from Cell Applications (San Diego, Calif.) and cultured in their proprietary HUVEC cell media containing 2% serum, 100 U/mL penicillin and 100 mg/mL streptomycin.

### Transfection

24 hours prior to transfection, HEK293 cells were plated into 96 well plates at 10,000 cells/well in 90 uL of antibiotic free media. The oligonucleotide or oligonucleotide conjugates were diluted in PBS to 100× of the desired final concentration. Separately, Lipofectamine RNAiMax (Life Technologies) was diluted 1:66.7 in media lacking supplements (e.g. FBS, antibiotic etc.). The 100× oligonucleotide in PBS was then complexed with RNAiMAX by adding 1 part oligonucleotide in PBS to 9 parts lipofectamine/media. Following incubation for 20 minutes, 10 uL of the oligonucleotide:RNAiMAX complexes were added to the cells plated 24 hours prior containing 90 uL of antibiotic free media. The complexes were removed 24 hours following and replaced with media containing antibiotics. RNA was isolated 48 hours following transfection.

### Free Uptake Experiments

HUVEC cells were plated at 10,000 cells/well on 96 well collagen-coated plates. The day after plating, the HUVEC media was removed and the cells were washed twice with PBS containing calcium and magnesium. Following the last wash, cells were incubated with compounds at various concentrations in serum free HUVEC media for 24 hours. After 24 hours, compound containing media was removed and replaced with normal HUVEC media for 24 additional hours. Cells were then washed twice with PBS containing calcium and magnesium and then prepared for RNA isolation according to the manufacturer's protocol (see above). In an alternative paradigm when the effect of albumin was not of interest, cells were incubated with compounds at various concentrations in normal HUVEC media containing 2% serum for 48 hours. After 48 hours, cells were washed twice with PBS containing calcium and magnesium and then prepared for RNA isolation according to the manufacturer's protocol (see below).

### Free Uptake Experiments in the Presence of Varying Concentrations of Albumin

HUVEC cells were plated at 10,000 cells/well on 96 well collagen-coated plates. The day after plating, the HUVEC media was removed and the cells were washed twice with PBS containing calcium and magnesium. Following the last wash, cells were incubated with compound for 24 hours in serum-free media lacking albumin or containing 0.03125%, 0.625%, 0.125%, 0.25% or 0.5% bovine serum albumin. After 24 hours, compound containing media was removed and replaced with normal HUVEC media for 24 additional hours. Cells were then washed twice with PBS containing calcium and magnesium and then prepared for RNA isolation according to the manufacturer's protocol (see below).

### RNA Isolation, Reverse Transcription and Quantitative PCR

RNA was isolated utilizing the RNeasy 96 kit (Qiagen) according to the manufacturer's protocol. It was reverse transcribed to cDNA utilizing random primers and the high-capacity cDNA reverse transcription kit (ThermoFisher Scientific) in a SimpliAmp thermal cycler (ThermoFisher Scientific) according to manufacturer's instructions. Quantitative PCR was performed utilizing gene-specific primers (Thermofisher Scientific; IDTDNA), TaqMan probes (Thermofisher Scientific; IDTDNA) and TaqMan fast universal PCR master mix (Thermofisher scientific) on a StepOnePlus real-time PCR system (Thermofisher scientific) according to manufacturer's instructions. For analysis of quantitative PCR, mRNA expression was normalized to the expression of either 18s rRNA, β-actin or HPRT1 mRNA (housekeeping genes) utilizing the relative CT method according to the best practices proposed in Nature Protocols (Schmittgen, T. D. & Livak, K. J. Analyzing real-time PCR data by the comparative C(T) method. Nat Protoc 3, 1101-1108 (2008)).

### Systemic Delivery Studies

Following acclimatization for 7 days, mice were weighed the night before the study and sorted into groups based on body weight. The day of study initiation, the mice were injected with PBS or the compound of interest via intravenous injection. The dosing paradigm and duration of study are provided in the biological data section. After a period of time, the mice were euthanized by CO₂ asphyxiation followed by secondary confirmation of euthanasia via cervical dislocation seven days following either a single injection or seven days following the last dose when repeated injections were utilized. The tissues of interest were then removed and 30-300 mg placed in RNALater immediately following dissection. 24 hours later, the tissue was removed from the RNALater, blotted dry and placed into trizol in tubes containing lysing matrix D beads from MPBiomedical. The tissue was homogenized using the MPBio FastPrep-24 system. Chloroform extraction was then performed by adding 0.2 mL per 1 mL of Trizol. Samples were mixed thoroughly, spun at max speed in a microcentrifuge at 4° C. for 15 minutes and the aqueous layer. The RNA was then precipitated by adding 1.5 volumes of absolute ethanol to the aqueous phase. The precipitated RNA was then purified utilizing the RNeasy 96 kit from Qiagen according to the manufacturer's instructions, substituting RLT buffer for RW1 buffer.

### Results

Selection of PTEN as siRNA Target

PTEN was chosen as the siRNA target because it is ubiquitously expressed across all cells and tissues and is a target that is commonly used to characterize new delivery technologies for siRNA and antisense molecules.

### Nucleic Acid Compounds Conjugated to Uptake and Half-Life Extension Motifs

Conjugation of certain long chain fatty acid (LCFA) motifs to siRNAs increases the uptake of the conjugated siRNA into cells, relative to unconjugated siRNA, without the aid of any transfection reagent. Such a motif is referred to herein as an “uptake motif.” For example, siRNA conjugated to the uptake motifs in Table A-0 including DTx-01-08 enables robust inhibition of the target mRNA both in vitro (in the presence and absence of transfection reagent) and in vivo.

Free uptake experiments were performed in HUVEC cells by incubating the compounds listed in Table A-0 for 48 hours across several distinct experiments (Tables A-1 to A-4). RNA was then isolated and the mean PTEN mRNA expression across 4 replicates per treatment quantified by QT-PCR. In all cases, the siRNAs containing the uptake motif repressed PTEN mRNA expression dose dependently whereas the unconjugated siRNA was far less potent and efficacious (Tables A-1 to A-4).

TABLE A-0
Examples of Uptake Motif siRNA Conjugates

| | | | Conju- | |
|---|---|---|---|---|
| | Uptake | | gation | |
| | Domain | Linker | Site | siRNA |
| DT-000155 | DTx-01-08 | C7 | 3′ | |
| DT-000175 | DTx-01-32 | C7 | 3′ | DTx-0038 |
| DT-000176 | DTx-01-33 | C7 | 3′ | DTx-0038 |
| DT-000177 | DTx-01-50 | C7 | 3′ | DTx-0038 |
| DT-000178 | DTx-01-51 | C7 | 3′ | DTx-0038 |
| DT-000179 | DTx-01-52 | C7 | 3′ | DTx-0038 |
| DT-000180 | DTx-01-54 | C7 | 3′ | DTx-0038 |
| DT-000181 | DTx-01-55 | C7 | 3′ | DTx-0038 |
| DTx-0038 | N/A | N/A | N/A | DTx-0038 |
| DT-000137 | DTx-01-08 | C7 | 3′ | DTx-0003 |
| DT-000149 | DTx-01-32 | C7 | 3′ | DTx-0003 |
| DT-000150 | DTx-01-33 | C7 | 3′ | DTx-0003 |
| DT-000166 | DTx-01-50 | C7 | 3′ | DTx-0003 |
| DT-000167 | DTx-01-51 | C7 | 3′ | DTx-0003 |
| DT-000168 | DTx-01-52 | C7 | 3′ | DTx-0003 |
| DT-000169 | DTx-01-53 | C7 | 3′ | DTx-0003 |
| DT-000170 | DTx-01-54 | C7 | 3′ | DTx-0003 |
| DT-000171 | DTx-01-55 | C7 | 3′ | DTx-0003 |
| DTx-0003 | N/A | N/A | N/A | DTx-0003 |

TABLE A-1
Free Uptake of Conjugated DTx-0038 PTEN siRNA in HUVEC Cells

| | | 3000 | 1000 | 300 | 100 | 30 | 10 | |
|---|---|---|---|---|---|---|---|---|
| | | nM | nM | nM | nM | nM | nM | 0 nM |
| PBS | Mean | | | | | | | 101 |
| | S.E.M. | | | | | | | 1.634 |
| DT-000155 | Mean | 8.038 | 1.276 | 9.101 | 23.55 | 47.17 | 90.84 | |
| | S.E.M. | 1.521 | 0.0601 | 1.121 | 0.4324 | 4.738 | 1.361 | |
| DT-000175 | Mean | 0.2545 | 0.5559 | 4.663 | 18.09 | 65.35 | 89.92 | |
| | S.E.M. | 0.03518 | 0.06072 | 0.5386 | 1.058 | 2.443 | 7.903 | |
| DT-000176 | Mean | 1.841 | 13.81 | 53.48 | 60.76 | 87.7 | 80.12 | |
| | S.E.M. | 0.1939 | 1.061 | 1.432 | 1.173 | 5.993 | 2.351 | |
| DT-000177 | Mean | 0.2545 | 0.3163 | 2.211 | 14.77 | 40.64 | 74.93 | |
| | S.E.M. | 0.0254 | 0.02867 | 0.2396 | 1.531 | 1.61 | 1.838 | |
| DT-000178 | Mean | 0.3601 | 0.4556 | 3.466 | 20.08 | 55.78 | 76.34 | |
| | S.E.M. | 0.04931 | 0.05902 | 0.4511 | 1.111 | 1.822 | 3.005 | |
| DT-000179 | Mean | 0.6218 | 3.246 | 34.45 | 74.16 | 95.58 | 106.7 | |
| | S.E.M. | 0.1083 | 0.3326 | 1.41 | 2.854 | 3.292 | 4.649 | |
| DT-000180 | Mean | 2.803 | 0.7823 | 9.561 | 27.8 | 93.84 | 86.73 | |
| | S.E.M. | 0.5419 | 0.06249 | 0.6731 | 1.088 | 2.387 | 2.494 | |
| DT-000181 | Mean | 1.736 | 1.042 | 7.511 | 30.19 | 70.22 | 86.51 | |
| | S.E.M. | 0.2451 | 0.08638 | 0.8459 | 1.783 | 1.812 | 5.071 | |
| DTx-0038 | Mean | 65.98 | 80.54 | 79.94 | 85.07 | 94.9 | 82.56 | |
| | S.E.M. | 4.486 | 4.458 | 2.022 | 1.69 | 4.489 | 4.969 | |

TABLE A-2
Free Uptake of Conjugated DTx-0003 PTEN siRNA in HUVEC Cells

| | | 1000 nM | 300 nM | 100 nM | 30 nM | 10 nM | 0 nM |
|---|---|---|---|---|---|---|---|
| PBS | Mean | | | | | | 100.1 |
| | S.E.M. | | | | | | 2.013 |
| DT-000137 | Mean | 11.65 | 15.7 | 27.63 | 55.96 | 85.59 | |
| | S.E.M. | 0.4 | 0.43 | 0.66 | 1.19 | 5.908 | |
| DT-000149 | Mean | 10.25 | 13.55 | 27.62 | 58.88 | 78.97 | |
| | S.E.M. | 0.534 | 0.445 | 1.175 | 1.067 | 1.817 | |
| DT-000166 | Mean | 11.06 | 14.18 | 17.54 | 40.01 | 65.58 | |
| | S.E.M. | 0.409 | 3.084 | 1.203 | 0.43 | 2.32 | |
| DT-000167 | Mean | 10.96 | 12.39 | 17.19 | 39.75 | 64.79 | |
| | S.E.M. | 0.843 | 0.546 | 0.431 | 1.529 | 3.385 | |
| DT-00003 | Mean | 94.64 | N.T. | 99.21 | N.T. | N.T. | |
| | S.E.M. | 1.912 | N.T. | 0.966 | N.T. | N.T. | |

TABLE A-3
Free Uptake of Conjugated DTx-0003 PTEN siRNA in HUVEC Cells

| | | | | | | 10 | |
|---|---|---|---|---|---|---|---|
| | | 1000 nM | 300 nM | 100 nM | 30 nM | nM | 0 nM |
| PBS | Mean | — | — | — | — | — | 100.1 |
| | S.E.M. | — | — | — | — | — | 1.99 |
| DT-000137 | Mean | 14.4 | 18.7 | 32.42 | 66.12 | 91.82 | — |
| | S.E.M. | 0.5134 | 0.2595 | 0.3392 | 2.332 | 2.828 | — |
| DT-000150 | Mean | 23.88 | 59.64 | 80.96 | 95.43 | 105.1 | — |
| | S.E.M. | 0.3894 | 2.479 | 2.642 | 2.24 | 3.061 | |
| DT-000168 | Mean | 15.37 | 27.85 | 47.55 | 73.72 | 91.1 | — |
| | S.E.M. | 0.7087 | 0.777 | 1.415 | 1.653 | 2.442 | |
| DT-000169 | Mean | 16.04 | 30.78 | 53.75 | 80.72 | 89.22 | — |
| | S.E.M. | 0.8565 | 0.9835 | 2.592 | 2.178 | 2.438 | |
| DT-00003 | Mean | 100.1 | N.T. | 100.8 | N.T. | N.T. | — |
| | S.E.M. | 2.984 | N.T. | 0.8872 | N.T. | N.T. | |

TABLE A-4
Free Uptake of Conjugated DTx-0003 PTEN siRNA in HUVEC Cells

| | | 1000 nM | 300 nM | 100 nM | 30 nM | 10 nM | 0 nM |
|---|---|---|---|---|---|---|---|
| PBS | Mean | — | — | — | — | — | 100.1 |
| | S.E.M. | — | — | — | — | — | 1.357 |
| DT- | Mean | 10.2 | 14.2 | 27.74 | 61.82 | 86.31 | — |
| 000149 | S.E.M. | 0.1473 | 0.5998 | 0.988 | 1.003 | 3.714 | — |
| DT- | Mean | 19.39 | 50.64 | 70.35 | 84.08 | 92.98 | — |
| 000150 | S.E.M. | 1.415 | 1.582 | 0.9226 | 1.767 | 1.759 | |
| DT- | Mean | 10.52 | 15.89 | 21.55 | 47.79 | 72.11 | — |
| 000170 | S.E.M. | 0.5894 | 1.208 | 0.6576 | 1.75 | 2.21 | |
| DT- | Mean | 9.928 | 15.59 | 20.73 | 42.72 | 69.77 | — |
| 000171 | S.E.M. | 0.5832 | 2.176 | 0.7722 | 1.657 | 1.538 | |
| DT-00003 | Mean | 94.18 | N.T. | 98.98 | N.T. | N.T. | — |
| | S.E.M. | 1.585 | N.T. | 3.688 | N.T. | N.T. | |

A subset of the compounds evaluated in Table A-1 were evaluated in vivo. Compounds DT-000155, DT-000175, DT-000176, DT-000177 and DT-000178 were tested in a first study. Compounds DT-000155, DT-000179, DT-000180, and DT-000183 were tested in a second study. C57Bl6/J mice were injected intravenously with a single dose of either PBS or 30 mg/kg of siRNAs containing uptake motifs. Seven days following injection, mice were euthanized and tissues extracted and RNA isolated. Mean repression of PTEN mRNA expression was calculated from 4-5 replicates per treatment. Many of the compounds dose dependently inhibited PTEN mRNA expression across the suite of tissues evaluated to a greater degree than mice treated with vehicle (Tables B1, C1 and D1 for the first study; Tables B2, C2 and D2 for the second study). These data demonstrate that uptake motifs improve the activity of siRNA in vivo.

TABLE B-1
Knockdown of PTEN mRNA in muscle
following a single intravenous injection of
DTx conjugated siRNA into C57Bl6/J mice

| | Vehicle | | 30 mg/kg | |
|---|---|---|---|---|
| | Mean | SEM | Mean | SEM |
| PBS | 100.6 | 3.57 | — | — |
| DT-000155 | — | — | 48.02 | 3.50 |
| DT-000175 | — | — | 43.61 | 2.55 |
| DT-000176 | — | — | 82.93 | 3.74 |
| DT-000177 | — | — | 46.48 | 2.52 |
| DT-000178 | — | — | 47.88 | 4.84 |

TABLE B-2
Knockdown of PTEN mRNA in muscle
following a single intravenous injection of
DTx conjugated siRNA into C57Bl6/J mice

| | Vehicle | | 30 mg/kg | |
|---|---|---|---|---|
| | Mean | SEM | Mean | SEM |
| PBS | 100.7 | 3.813 | — | — |
| DT-000155 | — | — | 52.26 | 4.17 |
| DT-000179 | — | — | 32.45 | 5.87 |
| DT-000180 | — | — | 22.57 | 5.30 |
| DT-000181 | — | — | 51.55 | 4.32 |

TABLE C-1
Knockdown of PTEN mRNA in heart
following a single intravenous injection of
DTx conjugated siRNA into C57Bl6/J mice

| | Vehicle | | 30 mg/kg | |
|---|---|---|---|---|
| | Mean | S.E.M | Mean | S.E.M |
| PBS | 100.7 | 4.1 | — | — |
| DT-000155 | — | — | 65.9 | 3.273 |
| DT-000175 | — | — | 61.66 | 4.989 |
| DT-000176 | — | — | 77.18 | 3.368 |
| DT-000177 | — | — | 73.7 | 3.896 |
| DT-000178 | — | — | 75.37 | 5.772 |

TABLE C-2
Knockdown of PTEN mRNA in heart
following a single intravenous injection of
DTx conjugated siRNA into C57Bl6/J mice

| | Vehicle | | 30 mg/kg | |
|---|---|---|---|---|
| | Mean | SEM | Mean | SEM |
| PBS | 100.8 | 4.371 | — | — |
| DT-000155 | — | — | 65.21 | 3.964 |
| DT-000179 | — | — | 74.63 | 3.708 |
| DT-000180 | — | — | 68.57 | 9.983 |
| DT-000181 | — | — | 65.79 | 5.032 |

TABLE D-1
Knockdown of PTEN mRNA in diaphragm
following a single intravenous injection of
DTx conjugated siRNA into C57Bl6/J mice

| | Vehicle | | 30 mg/kg | |
|---|---|---|---|---|
| | Mean | SEM | Mean | SEM |
| PBS | 100.5 | 3.379 | — | — |
| DT-000155 | — | — | 47.52 | 4.673 |
| DT-000175 | — | — | 45.78 | 3.941 |
| DT-000176 | — | — | 59.79 | 2.177 |
| DT-000177 | — | — | 56.8 | 1.808 |
| DT-000178 | — | — | 77.69 | 7.978 |

TABLE D-2
Knockdown of PTEN mRNA in diaphragm
following a single intravenous injection of
DTx conjugated siRNA into C57Bl6/J mice

| | Vehicle | | 30 mg/kg | |
|---|---|---|---|---|
| | Mean | SEM | Mean | SEM |
| PBS | 100.4 | 3.111 | — | — |
| DT-000155 | — | — | 49.01 | 4.934 |
| DT-000179 | — | — | 54.93 | 1.821 |
| DT-000180 | — | — | 66.1 | 7.977 |
| DT-000181 | — | — | 49.25 | 1.823 |

To identify motifs that interact with albumin and increase the in vivo half-life of siRNAs, half-life extension motifs were designed (see, e.g. Tables 1, 2 and 3). Certain of the half-life extension motifs described herein were tested for their ability to facilitate mRNA expression and albumin binding, when conjugated to an siRNA also having an uptake motif.

Compound DT-000146 is conjugated to the uptake domain DTx-01-08 to the 3′ end of the sense strand of a PTEN siRNA (DTxO-0003) and is also conjugated to the half-life extension motif including DTx-01-12 (see FIG. 1). To confirm that conjugation of motifs to both the 5′ and 3′ end of the sense did not affect the ability of the PTEN siRNA to repress mRNA, Compounds DT-000146, DT-000137 and the unconjugated PTEN siRNA DTx-0003 were transfected into HEK293 cells. PBS treatment was used as a control. Each compound treatment was performed for four replicates. A total of 16 replicates were tested for the PBS treatment. At 24 hours, RNA was then isolated and QT-PCR performed to evaluate PTEN mRNA expression, and the mean PTEN mRNA expression from the four replicates was calculated. Table E provides the mean percent PTEN mRNA expression, relative to PBS control, remaining following each treatment. As shown in Table A, all 3 compounds were active at inhibiting PTEN mRNA expression following transfection.

TABLE E
Transfection of Unconjugated and Conjugated siRNA into HEK293 Cells

| | | Compound Concentration | | | | | |
|---|---|---|---|---|---|---|---|
| Treatment | | 30 nM | 10 nM | 3 nM | 1 nM | 0.3 nM | 0 nM |
| DT-000137 | mean | 10.23 | 12.31 | 16.16 | 24.94 | 40.18 | |
| (uptake motif) | SEM | 1.057 | 0.865 | 1.098 | 1.674 | 2.844 | |
| DT-000146 | mean | 17.65 | 19.67 | 32.32 | 38.95 | 43.75 | |
| uptake motif) | | | | | | | |
| (HLEM motif + | SEM | 1.63 | 1.832 | 3.533 | 3.542 | 4.331 | |
| DTxO-0003 | mean | 16.44 | 14.62 | 19.71 | 32.49 | 65.95 | |
| (unconjugated) | SEM | 4.409 | 1.977 | 1.5 | 2.261 | 9.749 | |
| PBS | mean | | | | | | 101.5 |
| | SEM | | | | | | 6.759 |

To confirm that Compound DT-000146, like DT-000137, has the ability to enter cells without the aid of a transfection reagent under free uptake conditions, Compounds DT-000137, DT-000146 and DTxO-0003 were incubated at varying doses on HUVEC cells for 24 hours in serum free media. PBS treatment was used as a control. Each treatment was performed for four replicates. At 24 hours, the compounds were removed and the media replaced with normal HUVEC media. RNA was then isolated and QT-PCR performed to evaluate PTEN mRNA expression, and the mean PTEN mRNA expression from the four replicates was calculated. Table F provides the mean percent PTEN mRNA expression, relative to PBS control, remaining following each treatment. Under these conditions, DT-000146 was similarly active at repressing PTEN mRNA expression as DT-000137. In this experiment, in the absence of a transfection reagent, the unconjugated DTxO-0003 had no effect to inhibit PTEN mRNA expression.

TABLE F
Free Uptake of siRNA in HUVEC Cells

| | | Compound Concentration | | | | | |
|---|---|---|---|---|---|---|---|
| Treatment | | 1000 nM | 300 nM | 100 nM | 30 nM | 10 nM | 0 nM |
| DT-000137 | Mean | 10.55 | 14.14 | 26.58 | 56.47 | 83.48 | |
| (uptake motif) | SEM | 1.434 | 0.8127 | 0.8438 | 3.277 | 4.21 | |
| DT-000146 + | Mean | 10.49 | 16.25 | 31.92 | 68.53 | 92.46 | |
| uptake motif) | | | | | | | |
| (HELM motif | SEM | 0.6643 | 1.515 | 1.839 | 3.02 | 5.601 | |
| DTxO-0003 | Mean | 103.1 | | 105.5 | | 109.3 | |
| (unconjugated) | SEM | 2.474 | | 7.265 | | 6.454 | |
| PBS | Mean | | | | | | 100.1 |
| | SEM | | | | | | 1.831 |

To understand if the affinity of Compound DT-000146 for albumin is higher relative to DT-000137, 100 nM of Compounds DT-000146, DT-000137, and DTxO-0003 were incubated for 24 hours on HUVEC cells in serum-free media lacking albumin or containing increasing concentrations of albumin (0.03125% to 0.5%). As above, this experiment was done in the absence of transfection reagent. Each treatment was performed for four replicates. At 24 hours, the compounds were removed and the media replaced with normal HUVEC media. RNA was then isolated and QT-PCR performed to evaluate PTEN mRNA expression, and the mean PTEN mRNA expression from the four replicates was calculated. Table G provides the mean percent PTEN mRNA expression, relative to PBS control, remaining following each treatment. Whereas DT-000137 and Compound DT-000146 had similar effects to inhibit PTEN mRNA expression in the absence of albumin, Compound DT-000146 was much less effective at repressing PTEN mRNA expression relative to DT-000137 at all the albumin concentrations evaluated. Increased inhibition of the activity of Compound DT-000146 in the presence of albumin suggests that Compound DT-000146 has a higher affinity for albumin than DT-000137. As expected, the unconjugated siRNA DTxO-0038 had no effect to repress PTEN mRNA expression in the absence or presence of albumin.

TABLE G
Free Uptake in HUVEC Cells Incubated with
Varying Albumin Concentrations

| | | | DT-000146 | | | |
|---|---|---|---|---|---|---|
| | DT-000137 | | (HLEM motif + | | DTxO-0003 | |
| | (uptake motif) | | uptake motif) | | (unconjugated) | |
| Albumin | mean | SEM | mean | SEM | mean | SEM |
| 0% | 4.156 | 0.271 | 4.670 | 0.372 | 100.705 | 6.575 |
| 0.03125% | 13.121 | 1.196 | 49.744 | 1.140 | 100.915 | 7.899 |
| 0.0625% | 22.212 | 1.398 | 75.590 | 3.458 | 100.129 | 2.937 |
| 0.125% | 48.828 | 2.963 | 109.961 | 2.921 | 100.088 | 2.391 |
| 0.25% | 62.524 | 1.115 | 107.625 | 6.338 | 100.207 | 3.723 |
| 0.5% | 79.063 | 2.529 | 116.683 | 4.315 | 100.326 | 4.602 |

To evaluate the albumin binding and mRNA inhibition properties of a compound with the reverse attachment of the uptake motif and half-life extension motif, Compounds DT-000156 and DT-000157 were designed. In Compound DT-000156, the uptake motif (including DTx-01-08) was conjugated to the 5′ end of the sense strand of a PTEN siRNA and the HLEM motif (including DTx-01-12) was conjugated to the 3′ end of the sense strand of the PTEN siRNA. In Compound DT-000157, the uptake motif (including DTx-01-08) was conjugated to the 5′ end of the sense strand of a PTEN siRNA. For these compounds, the PTEN siRNA DTxO-0038 was used.

To determine the affinity of Compound DT-000156 for albumin relative to Compound DT-000157, 1000 nM of Compounds DT-000156, DT-000157 and DTxO-0038 were incubated for 24 hours on HUVEC cells in serum-free media lacking albumin or containing increasing concentrations of albumin (0.03125% to 0.5%). As above, this experiment was done in the absence of transfection reagent. Each treatment was performed for four replicates. At 24 hours, the compounds were removed and the media replaced with normal HUVEC media. RNA was then isolated and QT-PCR performed to evaluate PTEN mRNA expression, and the mean PTEN mRNA expression from the four replicates was calculated. Table H provides the mean percent PTEN mRNA expression, relative to PBS control, remaining following each treatment.

Whereas Compound DT-000157 (uptake motif only) and Compound DT-000156 (uptake motif and half-life extension motif) had similar effects to inhibit PTEN mRNA expression in the absence of albumin or at the lowest albumin concentration tested, Compound DT-000156 was much less effective at repressing PTEN mRNA expression relative to Compound DT-000157 at higher albumin concentrations evaluated. Increased inhibition of the activity of Compound DT-000156 in the presence of albumin suggests that Compound DT-000156 has a higher affinity for albumin than Compound DT-000157. However, the activity of DT-000156 was not inhibited to the same extent as that of DT-000146, suggesting that some albumin binding activity of DT-000156 was lost, relative to DT-000146. As expected, the unconjugated siRNA DTxO-0038 did not inhibit PTEN mRNA expression in the absence or presence of albumin.

TABLE H
Free Uptake in HUVEC Cells Incubated with Varying Albumin
Concentrations

| | | | DT-000156 | | | |
|---|---|---|---|---|---|---|
| | DT-000157 | | (HLEM motif + | | DTxO-0038 | |
| | (uptake motif) | | uptake motif) | | (unconjugated) | |
| Albumin | mean | SEM | mean | SEM | mean | SEM |
| 0% | 2.182 | 0.333 | 0.621 | 0.163 | 100.059 | 1.980 |
| 0.03125% | 5.915 | 1.011 | 6.773 | 0.960 | 100.024 | 1.254 |
| 0.0625% | 13.849 | 0.539 | 20.586 | 1.064 | 100.137 | 2.976 |
| 0.125% | 21.020 | 1.153 | 41.966 | 4.152 | 100.249 | 3.977 |
| 0.25% | 32.539 | 1.460 | 65.522 | 5.115 | 100.487 | 5.897 |
| 0.5% | 54.731 | 3.029 | 85.123 | 4.826 | 100.124 | 2.922 |

### Activity Following Intravitreal Administration

Compounds DT-000137 and DT-000146 were tested for activity in the eye. C57Bl/6 mice were injected via intravitreal injection with either water or 700 pmol of DT-000137, DT-000146, or DTx-0003. Seven days following injection, the mice were euthanized and the retina isolated. RNA was isolated from the retina, QT-PCR was performed and PTEN mRNA expression quantified relative to a housekeeping gene. As shown in Table I the activity of DT-000146 having both the HLEM and uptake motifs was comparable to that of DT-000137 having only the uptake motif. This data suggests that under in vivo conditions in which albumin binding is not expected to contribute to distribution or half-life (e.g. in the eye), the HLEM motif neither inhibits nor improves activity in combination with the uptake motif.

TABLE I
PTEN mRNA Expression Following Intravitreal Injection

| | | DT-000137 | DT-000146 | |
|---|---|---|---|---|
| | | (uptake | (HLEM motif + | DTxO-0003 |
| | PBS | motif) | uptake motif) | (unconjugated) |
| mean | 101.3 | 39.08 | 44.23 | 85.83 |
| SEM | 5.137 | 4.531 | 2.860 | 4.103 |

### Improved Activity of Half-Life Extension Motif Conjugates Following Systemic Administration

To evaluate the activity of compounds containing half-life extension motifs in vivo, mice were intravenously injected with three doses of PBS or 10 mg/kg of Compound DT-000156 or Compound DT-000157. PBS was used as a control treatment. Various tissues were collected seven days following the third injection, and RNA was isolated and reverse transcribed. QT-PCR was then performed to quantify PTEN mRNA expression, and the mean PTEN mRNA expression from replicates (n=5 for each compound treatment; n=8 to 10 for PBS treatment) was calculated. Table J provides the mean percent PTEN mRNA expression, relative to PBS control, remaining following each treatment.

Compound DT-000156, conjugated to both uptake and half-life extension motifs, exhibited improved activity, relative to Compound DT-000157, in heart, fat, diaphragm, spleen, kidney and lung. Activity of Compounds DT-000157 and Compound DT-000156 were similar in muscle. As expected due to the blood-brain barrier, neither compound exhibited inhibitory activity in the brain.

Taken together with the data in Table I demonstrating that a compound including an HLEM and an uptake motif are similarly active to a compound including only an uptake motif in the eye, the data in Table J suggest that the improved activity is due to the presence of the HLEM motif.

TABLE J
In vivo Inhibition of PTEN mRNA Following
Administration of Compounds with
Uptake and Half-Life Extension Motifs

| | Treatment | | | |
|---|---|---|---|---|
| | | DT-000157 | DT-000156 | |
| | | (uptake | (HLEM motif + | |
| Tissue | PBS | motif) | uptake motif) | |
| Liver | 101.7 | 16.96 | 13.36 | Mean |
| | 6.211 | 1.308 | 0.5161 | SEM |
| Heart | 101.1 | 92.42 | 62.85 | Mean |
| | 5.075 | 6.689 | 2.014 | SEM |
| Muscle | 102.6 | 67.95 | 72.99 | Mean |
| | 7.879 | 4.992 | 4.807 | SEM |
| Fat | 100.8 | 59.89 | 35.25 | Mean |
| | 5.048 | 6.453 | 1.782 | SEM |
| Diaphragm | 101.6 | 66.22 | 50.54 | Mean |
| | 5.63 | 5.762 | 3.567 | SEM |
| Spleen | 100.3 | 85 | 77.49 | Mean |
| | 2.593 | 2.883 | 3.478 | SEM |
| Kidney | 100.3 | 80.4 | 66.47 | Mean |
| | 2.657 | 2.034 | 1.835 | SEM |
| Lung | 100.3 | 80.4 | 66.47 | Mean |
| | 2.657 | 2.034 | 1.835 | SEM |
| Brain | 100.1 | 99.38 | 102.4 | Mean |
| | 1.601 | 2.421 | 2.607 | SEM |

### Altering Linkers, Conjugate Site and Fatty Acids to Affect Albumin Binding

Further compounds were designed to evaluate the effects of linker composition, site of attachment on the sense strand, and nature of fatty acid on albumin binding. The compounds are shown in FIGS. 1A-1O.

Compounds DT-000272, DT-000273, DT-000274, DT-000275, DT-000276, DT-000277 and DT-000278 were tested under transfection conditions in HEK293 cells, in the absence of albumin. Each compound treatment was performed in four replicates. At 48 hours, RNA was then isolated and QT-PCR performed to evaluate PTEN mRNA expression, and the mean PTEN mRNA expression from the four replicates was calculated. Table K provides the mean percent PTEN mRNA expression, relative to PBS control, remaining following each treatment.

TABLE K
Half-Life Extension Motif Compounds Transfected into HEK293 Cells

| | | 30 nM | 10 nM | 3nM | InM | 0.3 nM | 0.1 nM | 0 nM |
|---|---|---|---|---|---|---|---|---|
| PBS | Mean | — | — | — | — | — | — | 97.97 |
| | S.E.M. | — | — | — | — | — | — | 1.792 |
| DT-000272 | Mean | 23.84 | 22.49 | 41.39 | 58.78 | 74.25 | 81.59 | — |
| | S.E.M. | 2.249 | 0.344 | 1.843 | 4.341 | 4.853 | 1.191 | — |
| DT-000273 | Mean | 22.32 | 23.31 | 44.21 | 61.61 | 72.73 | 88.7 | — |
| | S.E.M. | 2.283 | 0.8081 | 2.981 | 4.408 | 1.121 | 1.806 | — |
| DT-000274 | Mean | 19.1 | 29.63 | 49.96 | 70.63 | 87.84 | 94.77 | — |
| | S.E.M. | 0.5287 | 2.174 | 3.746 | 4.924 | 1.802 | 1.617 | — |
| DT-000275 | Mean | 20.01 | 22.34 | 45.41 | 67.93 | 75.9 | 85.35 | — |
| | S.E.M. | 0.3908 | 1.107 | 1.323 | 4.948 | 2.163 | 3.058 | — |
| DT-000276 | Mean | 18.68 | 25.46 | 52.73 | 72.77 | 80.06 | 87.27 | — |
| | S.E.M. | 0.4813 | 1.4 | 2.843 | 3.093 | 0.9232 | 2.706 | — |
| DT-000277 | Mean | 22.27 | 18.83 | 53.41 | 72.39 | 76.99 | 88.18 | — |
| | S.E.M. | 1.326 | 1.01 | 4.188 | 7.048 | 0.9873 | 1.257 | — |
| DT-000278 | Mean | 20.46 | 20.63 | 41.38 | 59.34 | 81.53 | 83.46 | — |
| | S.E.M. | 0.79 | 0.7769 | 2.189 | 5.696 | 1.384 | 1.443 | — |
| DT-000155 | Mean | 18.97 | 21.26 | 47.05 | 69.98 | 72.88 | 82.01 | — |
| | S.E.M. | 0.6513 | 1.33 | 2.538 | 1.062 | 1.956 | 1.752 | — |
| DT-000157 | Mean | 20.08 | 18.34 | 39.14 | 55.85 | 81.86 | 88.2 | — |
| | S.E.M. | 1.34 | 0.5183 | 2.643 | 4.027 | 4.882 | 1.405 | — |
| DT-000156 | Mean | 16.41 | 18.95 | 42.79 | 60.81 | 69.77 | 81.3 | — |
| | S.E.M. | 0.1791 | 0.7763 | 3.376 | 4.649 | 1.619 | 2.885 | — |
| DTx-0038 | Mean | 23.71 | 40.19 | 67.59 | 83.07 | 99.74 | 100.4 | — |
| | S.E.M. | 1.135 | 0.7574 | 3.173 | 7.208 | 1.829 | 2.519 | — |

Compounds DT-000272, DT-000273, DT-000274, DT-000275, DT-000276, DT-000277 and DT-000278 were also tested under free uptake conditions in HUVEC cells, in the absence of albumin. These compounds contain distinct linkers relative to DT-000156. Also tested were DT-000155 (DTx-0038 with the uptake motif conjugated to the ′3 end of the sense strand), Compounds DT-000156 and DT-00157, and DTxO-0038. Compounds were incubated at concentrations of 30 nM, 100 nM, 300 nM, and 1000 nM on HUVEC cells in serum-free media lacking albumin. After 24 hours, the compounds were removed and the media replaced with normal HUVEC media. RNA was then isolated and QT-PCR performed to evaluate PTEN mRNA expression, and the mean PTEN mRNA expression from the four replicates was calculated. Table L provides the mean percent PTEN mRNA expression remaining following each treatment. Each conjugated compound exhibited dose-dependent inhibition of PTEN mRNA expression.

TABLE L
Half-Life Extension Motif Compound Activity in HUVEC Cells

| | | 1000 nM | 300 nM | 100 nM | 30 nM | 0 nM |
|---|---|---|---|---|---|---|
| DT-000272 | mean | 4.023 | 14.71 | 28.94 | 58.19 | |
| | SEM | 0.9129 | 1.487 | 0.5741 | 1.679 | |
| DT-000273 | mean | 2.719 | 17.68 | 31.72 | 62.1 | |
| | SEM | 0.2863 | 0.7797 | 3.026 | 2.91 | |
| DT-000274 | mean | 4.174 | 20.92 | 41.61 | 67.72 | |
| | SEM | 0.6073 | 0.9499 | 4.047 | 4.47 | |
| DT-000275 | mean | 2.429 | 19.95 | 25.59 | 68.21 | |
| | SEM | 0.6601 | 1.312 | 1.979 | 3.964 | |
| DT-000276 | mean | 1.874 | 25.94 | 39.55 | 82.98 | |
| | SEM | 0.1885 | 1.669 | 2.657 | 5.217 | |
| DT-000277 | mean | 5.318 | 16.53 | 30.68 | 78.38 | |
| | SEM | 0.4925 | 1.336 | 3.364 | 5.139 | |
| DT-000278 | mean | 3.83 | 17.68 | 25.67 | 52.17 | |
| | SEM | 0.8672 | 0.6715 | 1.478 | 2.228 | |
| DT-000155 | mean | 6.726 | 10.67 | 18.14 | 44.37 | |
| | SEM | 0.3916 | 0.3119 | 1.66 | 6.2 | |
| DT-000157 | mean | 7.561 | 13.53 | 29.47 | 54.49 | |
| | SEM | 0.6344 | 1.239 | 2.601 | 9.18 | |
| DT-000156 | mean | 4.429 | 15.9 | 31.1 | 68.93 | |
| | SEM | 0.6542 | 0.6215 | 4.966 | 0.2299 | |
| DTxO-0038 | mean | 92.7 | 97.29 | 93.49 | 97.56 | |
| | SEM | 3.157 | 4.21 | 6.05 | 4.193 | |
| PBS | mean | | | | | 106.9 |
| | SEM | | | | | 5.116 |

The same set of compounds was tested in HUVEC cells in the presence of varying concentrations of albumin. Compounds were incubated at a concentration of 300 nM for 24 hours on HUVEC cells in serum-free media with no albumin, 0.03125% albumin, 0.0625% albumin, or 0.125% albumin. Each treatment was performed in four replicates. At 48 hours, the compounds were removed and the media replaced with normal HUVEC media. RNA was then isolated and QT-PCR performed to evaluate PTEN mRNA expression, and the mean PTEN mRNA expression from the four replicates was calculated. Table M provides the mean percent PTEN mRNA expression remaining following each treatment. The activity of all of the compounds containing both a half-life extension motif and an uptake motif was attenuated relative to that of the compounds containing only the uptake motif (compare DT-000157 to DT-000272, DT-000273, DT-000274, DT-000275, and DT-000276; compare DT-000155 to DT-000277 and DT-000278). As in the albumin free experiments, all compounds behaved similarly in the absence of albumin.

TABLE M
Free Uptake of Half-Life Extension Motif
Compounds in the Presence of Albumin

| | 0.12500% | | 0.06250% | | 0.03125% | | 0% | |
|---|---|---|---|---|---|---|---|---|
| | mean | SEM | mean | SEM | mean | SEM | mean | SEM |
| DT-000272 | 74.76 | 12.99 | 74.5 | 3.626 | 47.94 | 4.978 | 3.868 | 0.9762 |
| DT-000273 | 88.79 | 7.255 | 102.1 | 3.721 | 73.96 | 2.417 | 7.112 | 0.8185 |
| DT-000274 | 81.85 | 4.246 | 95.14 | 9.706 | 67.58 | 5.949 | 8.989 | 0.6818 |
| DT-000275 | 93.32 | 10.3 | 101.2 | 3.757 | 75.92 | 9.759 | 7.354 | 0.7503 |
| DT-000276 | 99.26 | 8.936 | 128.2 | 13.87 | 78.5 | 6.694 | 13.84 | 0.5167 |
| DT-000277 | 84.93 | 4.603 | 83.02 | 3.179 | 57.6 | 3.561 | 5.813 | 0.6723 |
| DT-000278 | 59.28 | 6.825 | 70.25 | 2.167 | 49.99 | 4.41 | 6.053 | 1.293 |
| DT-000155 | 47.02 | 1.904 | 29.22 | 2.569 | 13.32 | 2.097 | 4.882 | 0.9676 |
| DT-000157 | 47.69 | 1.73 | 32.43 | 2.806 | 14.47 | 0.8225 | 4.883 | 0.5676 |
| DT-000156 | 74.07 | 2.712 | 87.2 | 8.552 | 63.22 | 3.838 | 4.34 | 0.855 |
| DTxO-0038 | 85.85 | 7.422 | 100.8 | 11.35 | 90.53 | 2.392 | 101.7 | 5.529 |
| PBS | 100.7 | 7.058 | 100.1 | 2.71 | 101.2 | 8.808 | 100.1 | 2.878 |

An additional study was performed to test the activity of DT-000350 in HUVEC cells in the presence of varying amounts of albumin. Compounds were incubated at a concentration of 1000 nM for 24 hours on HUVEC cells in serum-free media with no albumin, 0.03125%, 0.0625%, 0.125%, 0.25% and 0.5% albumin. Each treatment was performed in four replicates. At 48 hours, the compounds were removed and the media replaced with normal HUVEC media. RNA was then isolated and QT-PCR performed to evaluate PTEN mRNA expression, and the mean PTEN mRNA expression from the four replicates was calculated. Table N provides the mean percent PTEN mRNA expression remaining following each treatment. The activity of all of the compounds containing both a half-life extension motif and an uptake motif was attenuated relative to that of the compounds containing only the uptake motif (compare DT-000155 to DT-000272, DT-000273, DT-000274, and DT-00350).

TABLE N
Free Uptake of Half-Life Extension Motif
Compounds in the Presence of Albumin

| | | DT- | DT- | DT- | DT- | DT- |
|---|---|---|---|---|---|---|
| Albumin | | 000272 | 000273 | 000274 | 000350 | 000155 |
| 0.50% | mean | 91.4 | 92.1 | 99.2 | 99.5 | 47.4 |
| | SEM | 3.8 | 1.9 | 5 | 1.2 | 4.6 |
| 0.25% | mean | 72.1 | 81.9 | 98.3 | 72.6 | 27.4 |
| | SEM | 5.7 | 5.3 | 2.3 | 1.7 | 2.1 |
| 0.13% | mean | 29.5 | 42.6 | 87.8 | 37.6 | 24.2 |
| | SEM | 3 | 1.5 | 2.6 | 2.1 | 4.4 |
| 0.06% | mean | 34.3 | 34.8 | 56 | 38.2 | 31 |
| | SEM | 2.9 | 2.8 | 2.4 | 0.8 | 7.8 |
| 0.03% | mean | 27.1 | 32.9 | 38.9 | 36.7 | 22.9 |
| | SEM | 2.4 | 1.8 | 0.9 | 1.8 | 10.6 |
| 0% | mean | 15.5 | 18.6 | 30.3 | 13.2 | 30 |
| | SEM | 1.4 | 1 | 2.4 | 0.7 | 8.5 |

### Improved Activity of Half-Life Extension Motif Conjugates Following Systemic Administration

To evaluate the activity of additional half-life extension motifs in vivo, mice were intravenously injected once every other day with one 10 mg/kg dose or three 10 mg/kg doses of Compounds DT-000272, DT-000273, DT-000274, and DT-000350. Compound DT-000155, having only an uptake motif, was selected for comparison to compounds having both an uptake motif an a half-life extension motif. Also tested was DT-000183, a compound having a trientennary GaNAc motif linked to the 3′ end of the sense strand of DTx-0038 via a C7OH linker. PBS, one or three doses as appropriate, was used as a control treatment. Various tissues were collected seven days following the third injection, and RNA was isolated and reverse transcribed. QT-PCR was then performed to quantify PTEN mRNA expression, and the mean PTEN mRNA expression from replicates (n=5 for each compound treatment; n=5 for PBS treatment) was calculated. Table N provides the mean percent PTEN mRNA expression, relative to PBS control, remaining following each treatment.

Taken together, these data suggest that the linker of the HLEM affects the differential activity of compounds with an HLEM and uptake motif, relative to compounds with only an uptake motif.

TABLE O
In vivo Inhibition of PTEN mRNA
Following Administration of Compounds with
Uptake and Half-Life Extension Motifs

| | | Three Doses | | One Dose | |
|---|---|---|---|---|---|
| Treatment | Tissue | Mean | SEM | Mean | SEM |
| PBS | Heart | 101.2 | 8.2 | 100.3 | 3.7 |
| | Diaphragm | 100.0 | 1.2 | 100.2 | 2.8 |
| | Lung | 100.0 | 0.3 | 100.3 | 3.7 |
| | Kidney | 100.1 | 2.5 | 100.3 | 3.9 |
| | Muscle | 100.5 | 5.0 | 100.4 | 4.5 |
| | Spleen | 95.7 | 6.6 | 100.1 | 2.4 |
| | Liver | 100.1 | 2.2 | 100.3 | 4.1 |
| DT-000157 | Heart | 64.6 | 2.9 | 78.6 | 1.4 |
| | Diaphragm | 78.8 | 1.7 | 91.3 | 1.3 |
| | Lung | 62.6 | 1.6 | 81.2 | 5.6 |
| | Kidney | 88.2 | 3.6 | 97.7 | 2.4 |
| | Muscle | 60.4 | 2.8 | 80.9 | 4.6 |
| | Spleen | 101.6 | 4.7 | 96.7 | 3.2 |
| | Liver | 14.4 | 0.8 | 23.1 | 1.2 |
| DT-000183 | Heart | 101.3 | 9.3 | 105.6 | 5.4 |
| | Diaphragm | 99.7 | 4.7 | 104.8 | 2.2 |
| | Lung | 87.4 | 5.4 | 87.0 | 4.9 |
| | Kidney | 95.9 | 2.8 | 88.5 | 1.9 |
| | Muscle | 88.4 | 2.2 | 107.2 | 4.1 |
| | Spleen | 108.5 | 3.7 | 115.7 | 3.0 |
| | Liver | 22.5 | 0.2 | 36.5 | 1.6 |
| DT-000272 | Heart | 76.23 | 4.9 | 70.3 | 6.1 |
| | Diaphragm | 81.8 | 2.9 | 83.3 | 1.5 |
| | Lung | 66.0 | 2.2 | 63.3 | 5.7 |
| | Kidney | 106.4 | 0.8 | 89.0 | 3.9 |
| | Muscle | 69.5 | 1.7 | 86.3 | 6.2 |
| | Spleen | 97.2 | 3.2 | 99.9 | 4.5 |
| | Liver | 14.2 | 0.7 | 24.5 | 2.0 |
| DT-000273 | Heart | 64.5 | 4.3 | 71.2 | 6.8 |
| | Diaphragm | 76.4 | 0.5 | 84.4 | 1.0 |
| | Lung | 59.0 | 2.8 | 70.7 | 3.7 |
| | Kidney | 102.1 | 2.4 | 91.8 | 3.1 |
| | Muscle | 65.7 | 2.2 | 82.3 | 7.9 |
| | Spleen | 96.1 | 3.2 | 92.8 | 3.7 |
| | Liver | 13.0 | 0.5 | 22.5 | 1.1 |
| DT-000274 | Heart | 80.8 | 5.3 | 91.4 | 9.5 |
| | Diaphragm | 80.4 | 1.7 | 89.8 | 1.9 |
| | Lung | 70.1 | 4.5 | 79.3 | 3.9 |
| | Kidney | 109.5 | 3.6 | 105.8 | 4.2 |
| | Muscle | 79.1 | 4.3 | 90.6 | 3.6 |
| | Spleen | 97.5 | 3.1 | 85.8 | 2.4 |
| | Liver | 13.3 | 0.5 | 18.9 | 1.9 |
| DT-000350 | Heart | 78.1 | 4.5 | 79.4 | 6.0 |
| | Diaphragm | 82.2 | 2.8 | 92.0 | 1.8 |
| | Lung | 71.6 | 4.6 | 80.0 | 6.0 |
| | Kidney | 113.9 | 6.1 | 107.7 | 1.2 |
| | Muscle | 79.8 | 4.8 | 83.2 | 6.2 |
| | Spleen | 94.2 | 1.6 | 92.2 | 3.6 |
| | Liver | 14.9 | 0.7 | 22.5 | 0.7 |

Although the disclosure has been described with reference to embodiments and examples, it should be understood that numerous and various modifications can be made without departing from the spirit of the present disclosure.

1. A compound comprising a nucleic acid (A) covalently bonded to a half-life extension motif (HLEM), wherein the compound has a formula (I):
(HLEM)_{z}-A (I)
wherein z is an integer from 1 to 5; and wherein
the half-life extension motif has the structure: 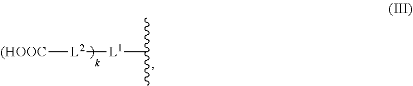
wherein:
L¹ is independently a covalent linker of the formula -L^{1A}-L^{1B}-L^{1C}-L^{1D}-L^{1E}-;
L^{1A}, L^{1B}, L^{1C}, L^{1D}, and L^{1E} are independently a bond, —N(R²⁰)—, —O—, —S—, —C(O)—, —N(R²⁰)C(O)—, —C(O)N(R²¹)—, —N(R²⁰)C(O)N(R²¹)—, —C(O)O—, —OC(O)—, —N(R²⁰)C(O)O—, —OC(O)N(R²¹)—, —OPO₂—O—, —O—P(O)(S)—O—, —O—P(O)(R²²)—O—, —O—P(S)(R²²)—O—, —O—P(O)(NR²⁰R²¹)—N—, —O—P(S)(NR²⁰R²¹)—N—, —O—P(O)(NR²⁰R²¹)—O—, —O—P(S)(NR²⁰R²¹)—O—, —P(O)(NR²⁰R²¹)—N—, —P(S)(NR²⁰R²¹)—N—, —P(O)(NR²⁰R²¹)—O—, —P(S)(NR²⁰R²¹)—O—, —S—S—, substituted or unsubstituted alkylene, substituted or unsubstituted heteroalkylene, substituted or unsubstituted cycloalkylene, or substituted or unsubstituted arylene;
each R²⁰, R²¹, and R²² is independently hydrogen or unsubstituted C₁-C₁₀ alkyl;
L² is independently an unsubstituted alkylene; and
k is an integer from 1 to 5.

2.-34. (canceled)

35. The compound of claim 1, wherein -L^{1A}-L^{1B}- is independently

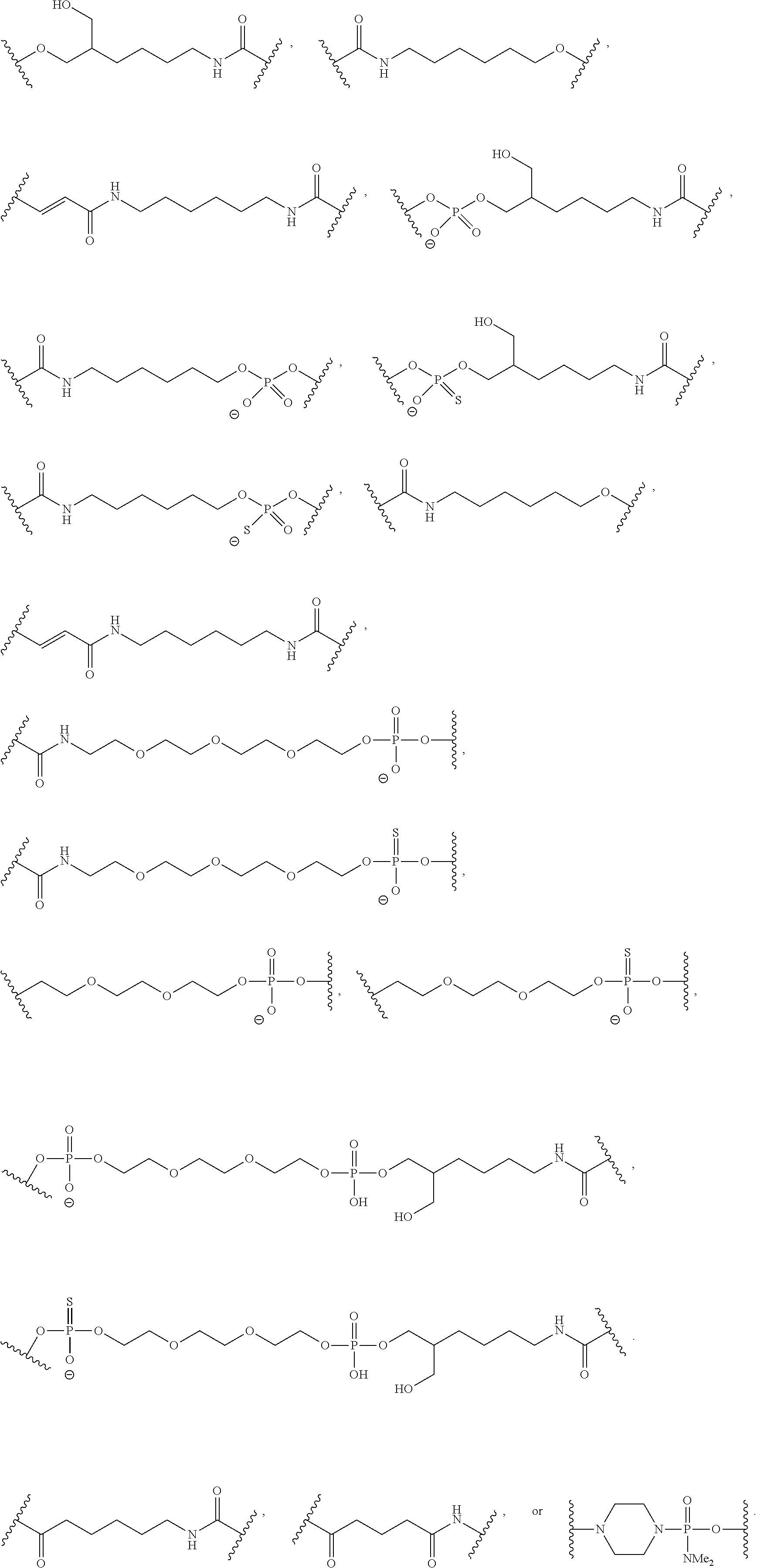

36.-46. (canceled)

47. The compound of claim 1, wherein the half-life extension motif has the structure: 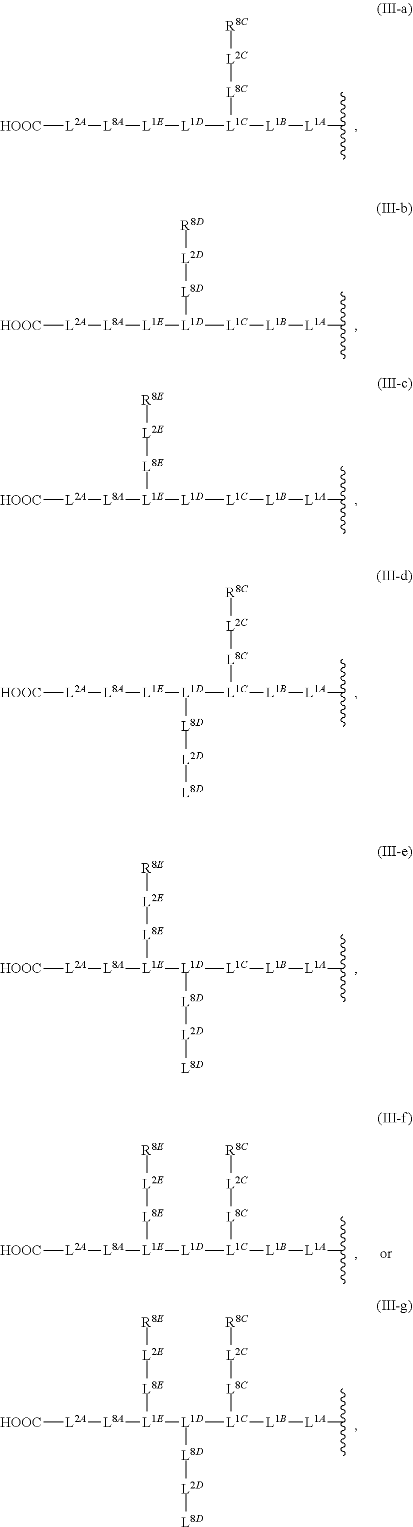
L^{1C} is independently R^{1C}-substituted or unsubstituted C₁-C₇ alkylene, or R^{1C}-substituted or unsubstituted 5 to 8 membered heteroalkylene;
L^{1D} is independently a bond, R^{1D}-substituted or unsubstituted C₁-C₇ alkylene, or R^{1D}-substituted or unsubstituted 5 to 8 membered heteroalkylene; and
L^{1E} is independently a bond, R^{1E}-substituted or unsubstituted 5 to 8 membered heteroalkylene, or —NHC(O)—;
R^{1C} is independently oxo, or -L^{8C}-L^{2C}-R^{8C};
R^{1D} is independently oxo, or -L^{8D}-L^{2D}-R^{8D};
R^{1E} is independently oxo, or -L^{8E}-L^{2E}-R^{8E};
L^{2A} is independently a bond, or an unsubstituted alkylene;
L^{8A} is independently a bond, substituted or unsubstituted alkylene, or substituted or unsubstituted heteroalkylene;
each L^{8C}, L^{8D}, and L^{8E} is independently a bond, substituted or unsubstituted C₁-C₆ alkylene, or substituted or unsubstituted 2 to 6 membered heteroalkylene;
each L^{2C}, L^{2D}, and L^{2E} is independently a bond, or an unsubstituted alkylene; and
each R^{8C}, R^{8D}, and R^{8E} is independently hydrogen, substituted or unsubstituted alkyl, or substituted or unsubstituted heteroalkyl.

48.-66. (canceled)

67. The compound of claim 1, wherein L¹ is:

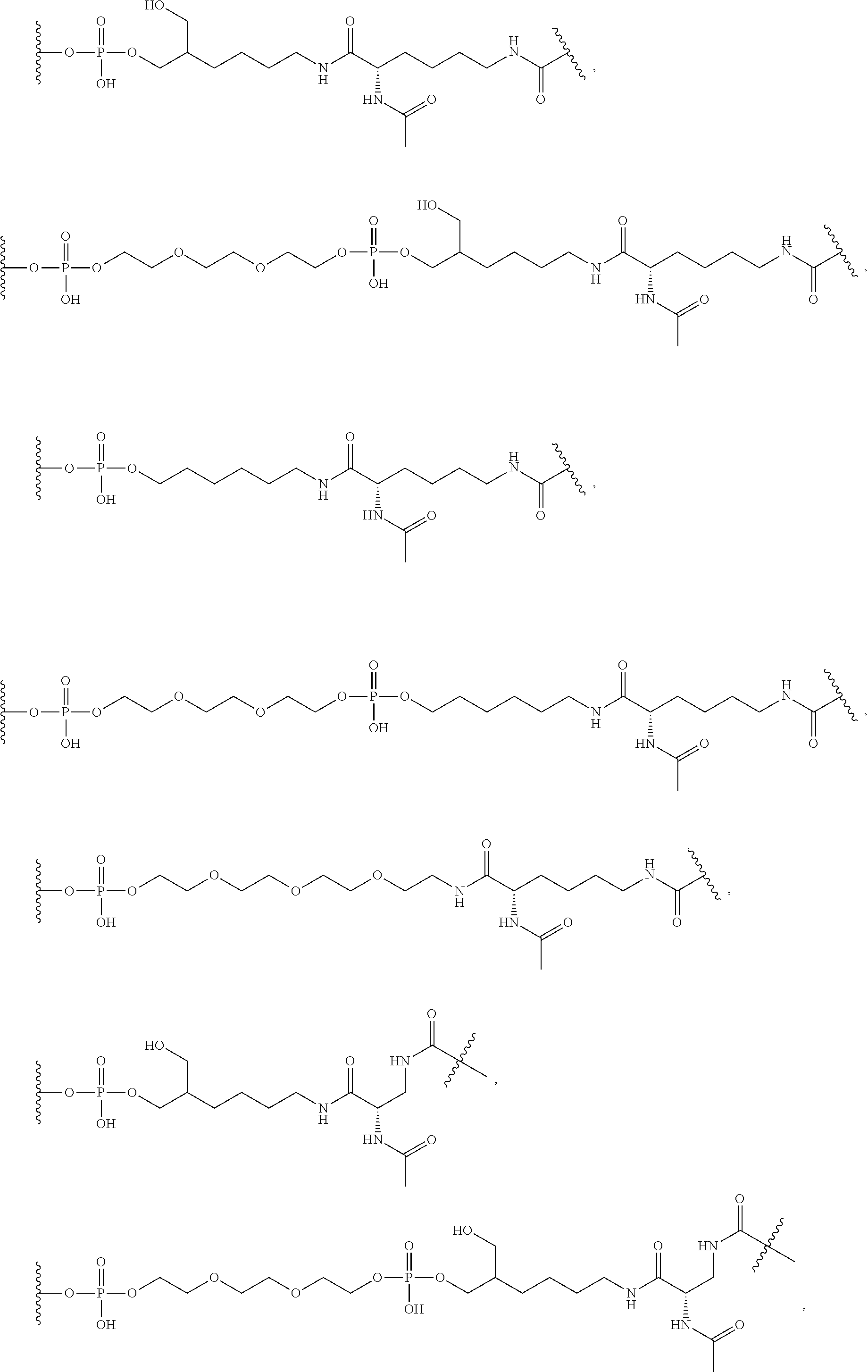

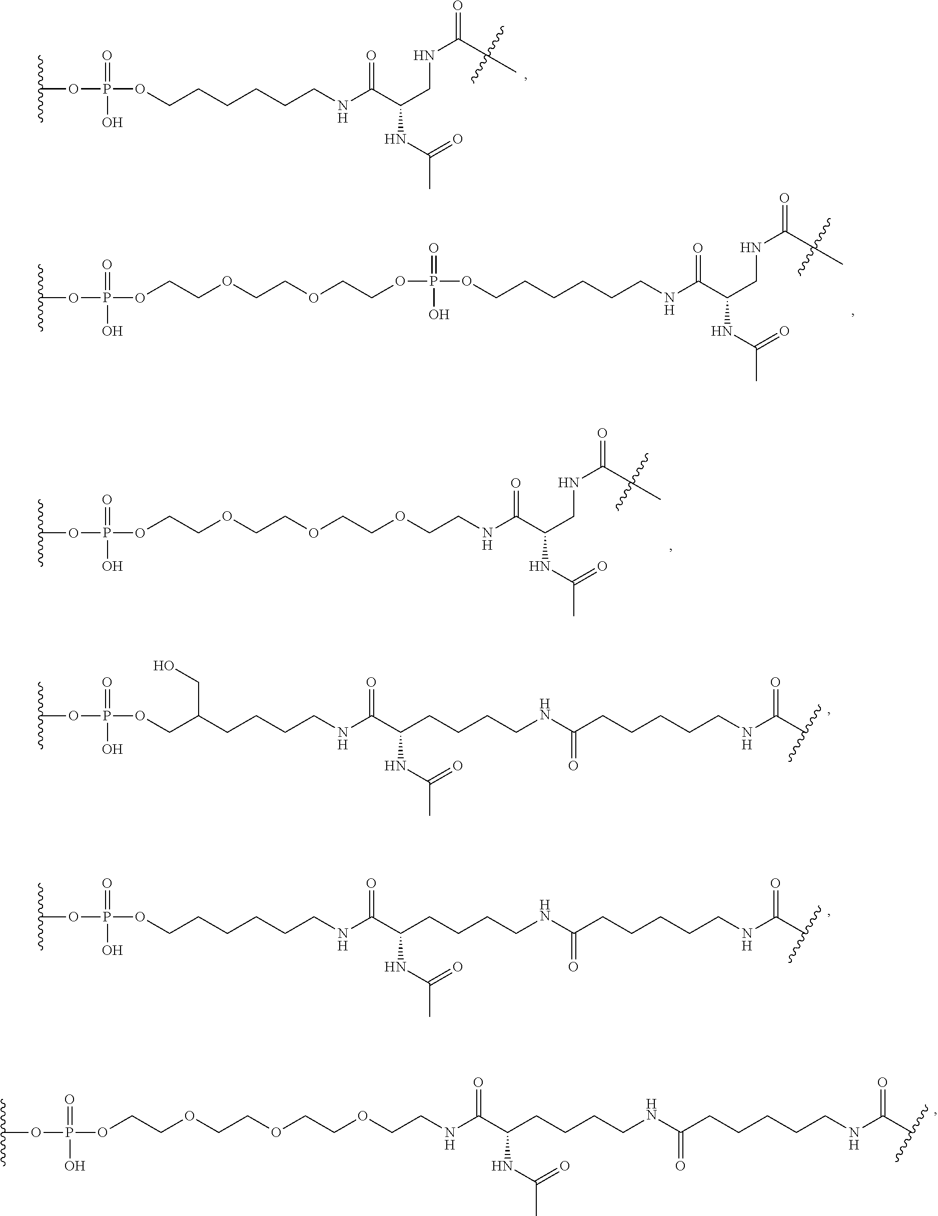 [SMILES: *OP(=O)(O)OCCCCCCNC(=O)[C@H](CNC(*)=O)NC(C)=O ; *OP(=O)(O)OCCOCCOCCOP(=O)(O)OCCCCCCNC(=O)[C@H](CNC(*)=O)NC(C)=O ; *OP(=O)(O)OCCOCCOCCOCCNC(=O)[C@H](CNC(*)=O)NC(C)=O ; *OP(=O)(O)OCC(CO)CCCCNC(=O)[C@H](CCCCNC(=O)CCCCCNC(*)=O)NC(C)=O ; *OP(=O)(O)OCCCCCCNC(=O)[C@H](CCCCNC(=O)CCCCCNC(*)=O)NC(C)=O ; *OP(=O)(O)OCCOCCOCCOCCNC(=O)[C@H](CCCCNC(=O)CCCCCNC(*)=O)NC(C)=O]

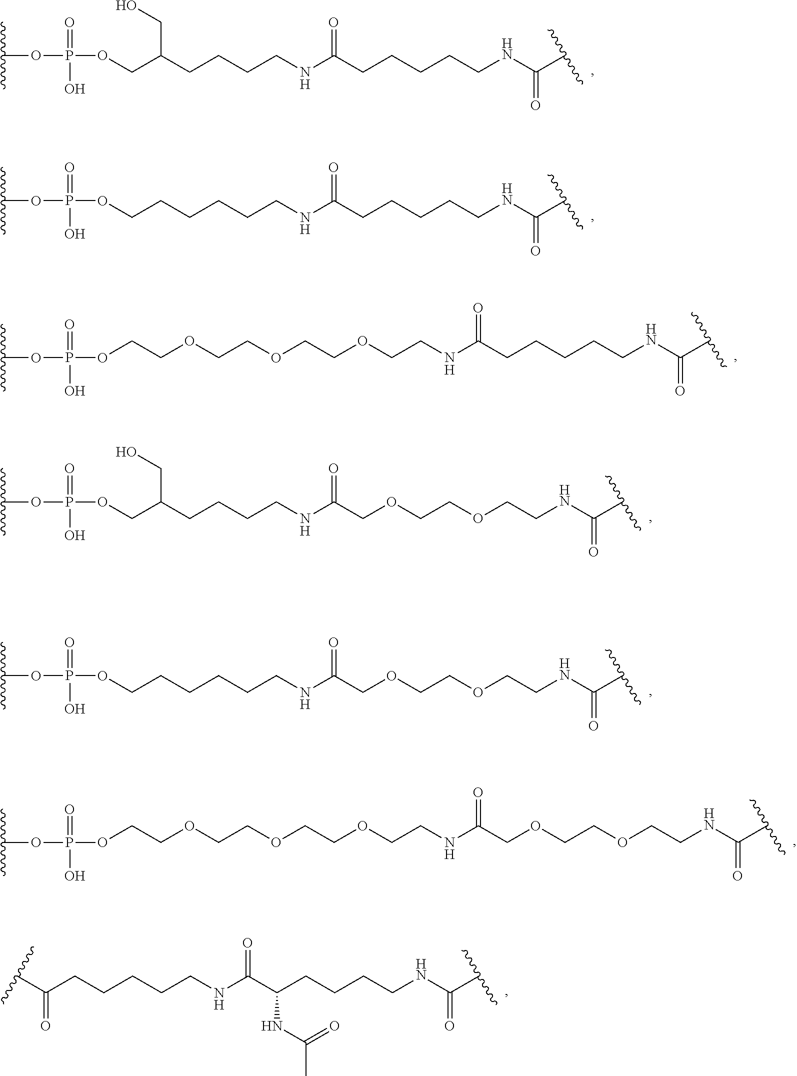

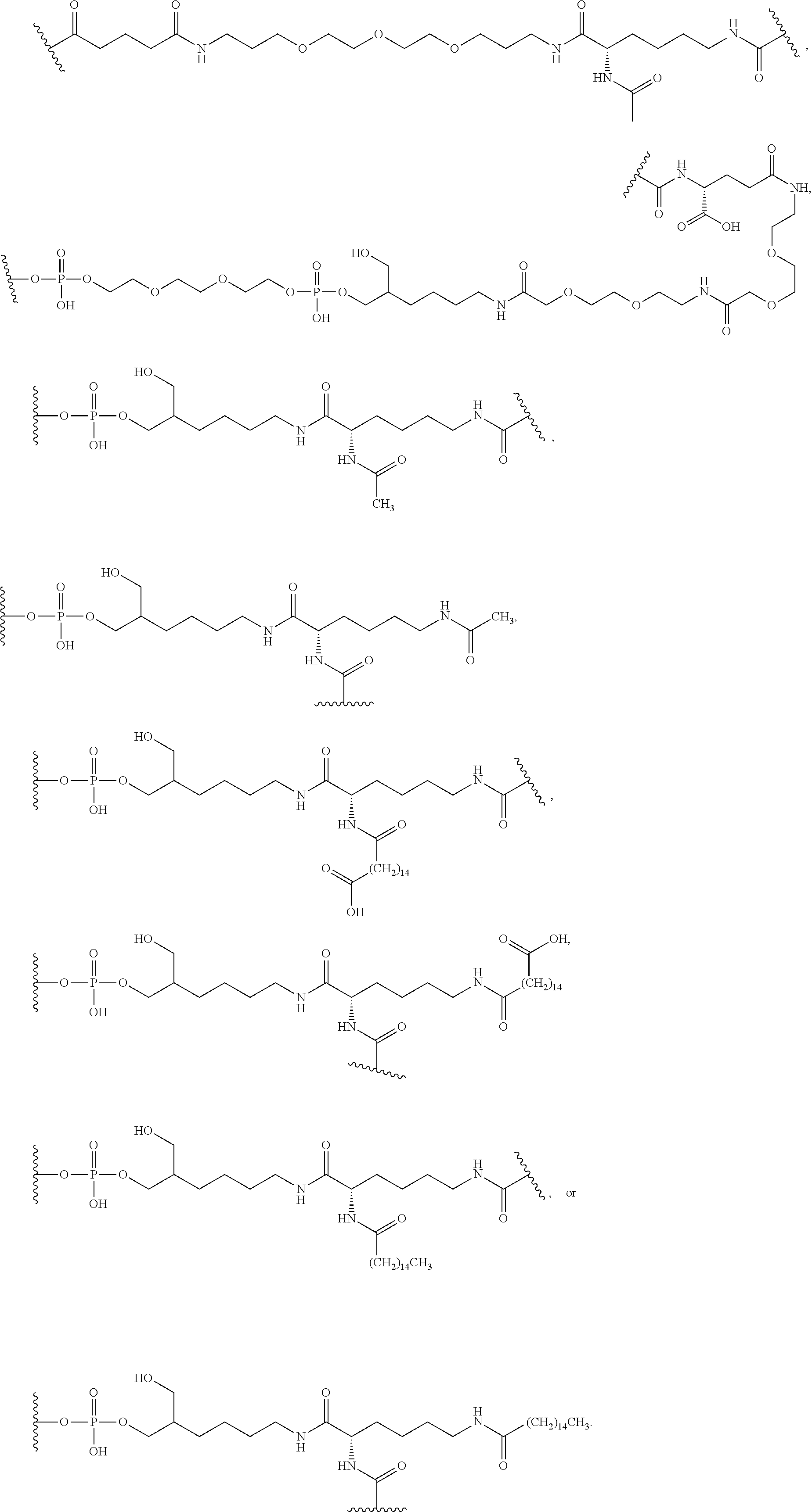

68.-86. (canceled)

87. The compound of claim 1, wherein the compound has a formula (II):
(HLEM)_{z}-A-(UM)ₜ (II),
wherein t is an integer from 1 to 5 and UM is an uptake motif covalently bonded to A; and wherein
the uptake motif independently has the structure: 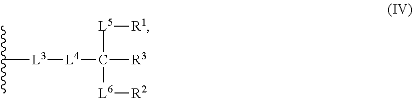
wherein:
L³ and L⁴ are independently a bond, —N(R²³)—, —O—, —S—, —C(O)—, —N(R²³)C(O)—, —C(O)N(R²⁴)—, —N(R²³)C(O)N(R²⁴)—, —C(O)O—, —OC(O)—, —N(R²³)C(O)O—, —OC(O)N(R²⁴)—, —OPO₂—O—, —O—P(O)(S)—O—, —O—P(O)(R²⁵)—O—, —O—P(S)(R²⁵)—O—, —O—P(O)(NR²³R²⁴)—N—, —O—P(S)(NR²³R²⁴)—N—, —O—P(O)(NR²³R²⁴)—O—, —O—P(S)(NR²³R²⁴)—O—, —P(O)(NR²³R²⁴)—N—, —P(S)(NR²³R²⁴)—N—, —P(O)(NR²³R²⁴)—O—, —P(S)(NR²³R²⁴)—O—, —S—S—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene;
L⁵ is -L^{5A}-L^{5B}-L^{5C}-L^{5D}-L^{5E}-;
L⁶ is -L^{6A}-L^{6B}-L^{6C}-L^{6D}-L^{6E}-;
R¹ and R² are independently unsubstituted C₁-C₂₅ alkyl, wherein at least one of R¹ and R² is unsubstituted C₉-C₁₉ alkyl;
R³ is hydrogen, —NH₂, —OH, —SH, —C(O)H, —C(O)NH₂, —NHC(O)H, —NHC(O)OH, —NHC(O)NH₂, —C(O)OH, —OC(O)H, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L^{5A}, L^{5B}, L^{5C}, L^{5D}, L^{5E}, L^{6A}, L^{6B}, L^{6C}, L^{6D}, and L^{6E} are independently a bond, —NH—, —O—, —S—, —C(O)—, —NHC(O)—, —NHC(O)NH—, —C(O)O—, —OC(O)—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene; and
each R²³, R²⁴ and R²⁵ is independently hydrogen or unsubstituted C₁-C₁₀ alkyl.

88.-108. (canceled)

109. The compound of claim 87, wherein -L³-L⁴- is independently

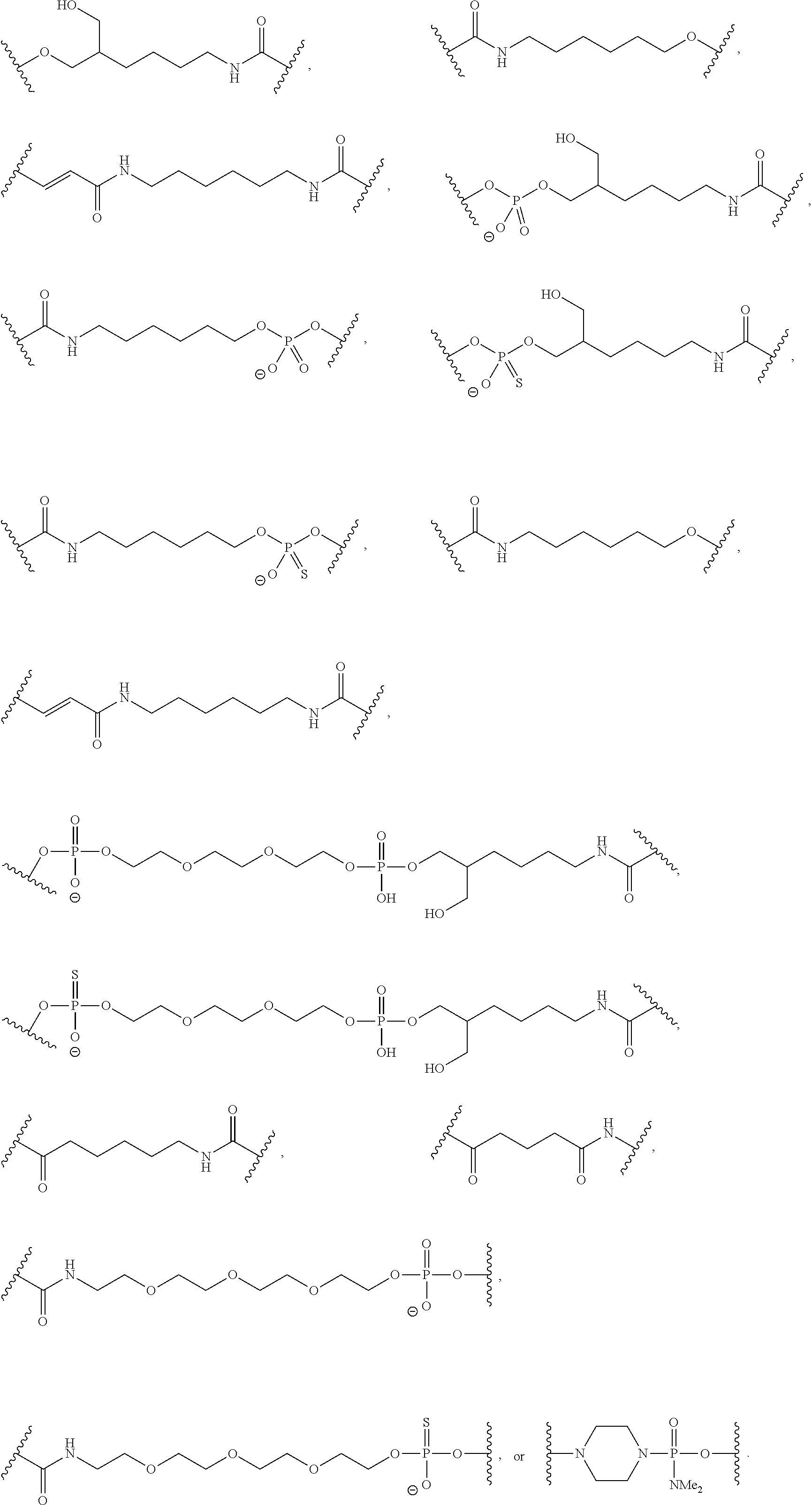

110.-119. (canceled)

120. The compound of claim 87, wherein L⁶ is independently a bond, —NHC(O)—,

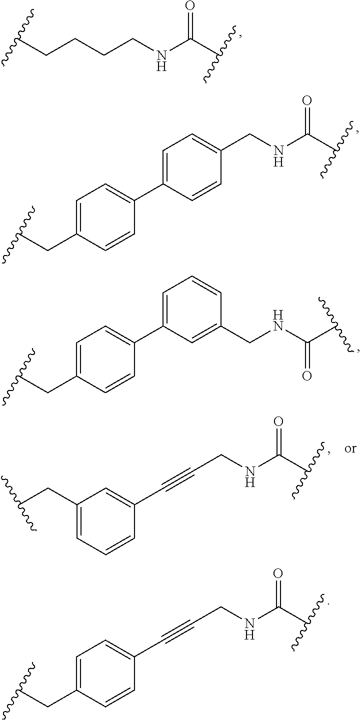 [SMILES: *CCCCNC(*)=O ; *Cc1ccc(-c2ccc(CNC(*)=O)cc2)cc1 ; *Cc1ccc(-c2cccc(CNC(*)=O)c2)cc1 ; *Cc1cccc(C#CCNC(*)=O)c1 ; *Cc1ccc(C#CCNC(*)=O)cc1]

121.-124. (canceled)

125. The compound of claim 87, wherein L⁵ is independently a bond, —NHC(O)—,

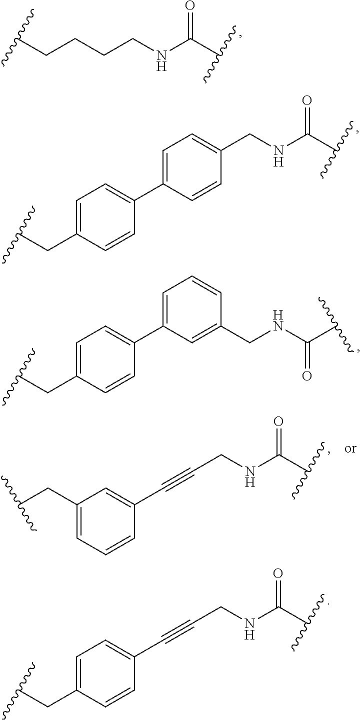 [SMILES: *CCCCNC(*)=O ; *Cc1ccc(-c2ccc(CNC(*)=O)cc2)cc1 ; *Cc1ccc(-c2cccc(CNC(*)=O)c2)cc1 ; *Cc1cccc(C#CCNC(*)=O)c1 ; *Cc1ccc(C#CCNC(*)=O)cc1]

126.-172. (canceled)

173. The compound of claim 1, wherein the compound is capable of binding a serum protein.

174.-176. (canceled)

177. The compound of claim 1, wherein the compound further comprises a ligand, wherein the ligand comprises a peptide, an antibody, a carbohydrate, or an additional nucleic acid.

178. (canceled)

179. The compound of claim 1, wherein the uptake motif comprises a peptide, an antibody, a carbohydrate, or an additional nucleic acid.

180. A method comprising contacting a cell with a compound of claim 1.

181.-183. (canceled)

184. A method comprising administering to a subject a compound of claim 1, wherein the subject has a disease or disorder of the eye, liver, kidney, heart, adipose tissue, lung, muscle or spleen.

185.-187. (canceled)

188. A method of introducing a nucleic acid into a cell within a subject, the method comprising administering to said subject the compound of claim 1.

189. A cell comprising the compound of claim 1.

190. A pharmaceutical composition comprising a pharmaceutically acceptable excipient and the compound of claim 1.

191. The compound of claim 1, wherein L^{1A} is independently —O—, —C(O)—, —C(O)O—, —OC(O)—, —OPO₂—O—, —O—P(O)(S)—O—, —O—P(O)(CH₃)—O—, —O—P(S)(CH₃)—O—, —O—P(O)(N(CH₃)₂)—N—, —O—P(O)(N(CH₃)₂)—O—, —O—P(S)(N(CH₃)₂)—N—, —O—P(S)(N(CH₃)₂)—O—, —P(O)(N(CH₃)₂)—N—, —P(O)(N(CH₃)₂)—O—, —P(S)(N(CH₃)₂)—N—, —P(S)(N(CH₃)₂)—O—, substituted or unsubstituted alkylene, or substituted or unsubstituted heteroalkylene.

192. The compound of claim 1, wherein L^{1B} is independently -L¹⁰-NH—C(O)— or -L¹⁰-C(O)—NH—, wherein L¹⁰ is substituted or unsubstituted alkylene.

193. The compound of claim 87, wherein L³ and L⁴ are independently a bond, —NH—, —O—, —C(O)—, —C(O)O—, —OC(O)—, —OPO₂—O—, —O—P(O)(S)—O—, —O—P(O)(CH₃)—O—, —O—P(S)(CH₃)—O—, —O—P(O)(N(CH₃)₂)—N—, —O—P(O)(N(CH₃)₂)—O—, —O—P(S)(N(CH₃)₂)—N—, —O—P(S)(N(CH₃)₂)—O—, —P(O)(N(CH₃)₂)—N—, —P(O)(N(CH₃)₂)—O—, —P(S)(N(CH₃)₂)—N—, —P(S)(N(CH₃)₂)—O—, substituted or unsubstituted alkylene, or substituted or unsubstituted heteroalkylene.

194. The compound of claim 87, wherein L⁶ is independently —NHC(O)—, —C(O)NH—, substituted or unsubstituted alkylene, or substituted or unsubstituted heteroalkylene.


SEQUENCE LISTING (4 sequences)

SEQ ID NO: 1 - DNA, 21 bp, Artificial Sequence
  Oligonucleotide
  misc_feature (1)..(19): RNA
gaugauguuugaaacuauutt

SEQ ID NO: 2 - DNA, 21 bp, Artificial Sequence
  Oligonucleotide
  misc_feature (1)..(19): RNA
aauaguuucaaacaucauctt

SEQ ID NO: 3 - RNA, 19 nt, Artificial Sequence
  Oligonucleotide
accugaucauuauagauaa

SEQ ID NO: 4 - DNA, 21 bp, Artificial Sequence
  Oligonucleotide
  misc_feature (2)..(19): RNA
tuaucuauaaugaucaggutt